(19) United States
(12) Patent Application Publication
(10) Pub. No.: US 2026/0176620 A1
(43) Pub. Date: Jun. 25, 2026

(54) RNA AGENTS TARGETING BCL11A AND RELATED METHODS

(71) Applicant: Marrow Therapeutics, Inc., Watertown, MA (US)

(72) Inventors: Romesh Ramsunder SUBRAMANIAN, Framingham, MA (US); Timothy Everett WEEDEN, Sturbridge, MA (US); Duncan Parley BROWN, Berkeley, CA (US); James Anthony MCSWIGGEN, Arvada, CO (US); Kathrin Stefani GERAUER, Prichsenstadt (DE); Jochen DECKERT, Bayreuth (DE)

(21) Appl. No.: 19/349,289

(22) Filed: Oct. 3, 2025

Related U.S. Application Data
(60) Provisional application No. 63/703,718, filed on Oct. 4, 2024.
(60) Provisional application No. 63/746,950, filed on Jan. 18, 2025.

(51) Int. Cl. C12N 15/11; A61K 31/713; C12N 15/113; C12N 15/63

(52) U.S. Cl. CPC C12N 15/111; A61K 31/713; C12N 15/113; C12N 15/1137; C12N 15/63; C12N 2310/14; C12N 2310/315; C12N 2310/3521

(57) ABSTRACT

Provided herein are, inter alia, agents (e.g., dsRNA agents comprising a sense strand and an antisense strand; RNAi agents, ssRNAs, ASOs) comprising a sense strand and an antisense strand targeting BCL11A (e.g., hBCL11A); and methods of manufacturing and pharmaceutical compositions comprising the same. Further provided herein are methods of utilizing the agents (e.g., dsRNA agents comprising a sense strand and an antisense strand; RNAi agents, ssRNAs, ASOs) including, e.g., methods of inhibiting or decreasing BCL11A expression (e.g., mRNA expression), methods of treating BCL11A associated diseases.

### RELATED APPLICATIONS

This application claims priority to U.S. Ser. No. 63/703,718, filed Oct. 4, 2024; and U.S. Ser. No. 63/746,950, filed Jan. 18, 2025, the entire contents of each of which is incorporated herein by reference.

### SEQUENCE LISTING

The instant application contains a Sequence Listing which has been submitted electronically in XML format and is hereby incorporated by reference in its entirety. Said XML copy, created on Feb. 19, 2026, is named 66504_27US01_SL.xml and is 24,543,232 bytes in size.

### 1. FIELD

This disclosure relates to RNA agents (e.g., double stranded RNA (dsRNA) agents comprising a sense strand and an antisense strand; RNAi agents, single stranded RNAs (ssRNAs), antisense oligonucleotides (ASOs), etc.) targeting BCL11 transcription factor A (BCL11A). The disclosure further relates to pharmaceutical compositions comprising the same; and methods of utilizing the same, including, e.g., methods of treating BCL11A associated diseases.

### 2. BACKGROUND

Transcription factor B-cell lymphoma/leukemia 11A (BCL11A) gene encodes a zinc-finger protein. BCL11A, e.g., associates with the SWI/SNF complex, that regulates gene expression via chromatin remodeling. BCL11A is highly expressed in several hematopoietic lineages, and plays a role in the switch from γ- to β-globin expression during the fetal to adult erythropoiesis transition.

### 3. SUMMARY

Provided herein are, inter alia, agents (e.g., RNA agents, RNAi agents, dsRNA agents, siRNA agents, ssRNA agents, antisense oligonucleotides, etc.) comprising a sense strand and an antisense strand targeting BCL11A (e.g., hBCL11A); and methods of manufacturing and pharmaceutical compositions comprising the same. Further provided herein are methods of utilizing the agents (e.g., RNA agents, RNAi agents, dsRNA agents, siRNA agents, ssRNA agents, antisense oligonucleotides, etc.) including, e.g., methods of inhibiting or decreasing BCL11A expression (e.g., mRNA expression), methods of treating BCL11A associated diseases.

Accordingly, in one aspect, provided herein dsRNA agents for inhibiting expression of BCL11A (e.g., hBCL11A), wherein the dsRNA agent comprises a sense strand and an antisense strand forming a double stranded region, wherein the nucleotide sequence of the antisense strand differs by no more than 5 (e.g., 0, 1, 2, 3, 4, or 5) nucleotides from the nucleotide sequence of any one of the antisense strands set forth in any one of Tables 2-5 or set forth in any one of SEQ ID NOS: 4-1576 (e.g., any one of SEQ ID NOS: 4-19 or 772-787).

In some embodiments, the nucleotide sequence of the sense strand differs by no more than 5 (e.g., 0, 1, 2, 3, 4, or 5) nucleotides from the nucleotide sequence of the corresponding sense strand set forth in Tables 2-5 or the corresponding sense strand set forth in one of SEQ ID NOS: 1577-3166 (e.g., any one of SEQ ID NOS: 1577-1592 or 2346-2361).

In some embodiments, the nucleotide sequence of the antisense strand differs by no more than 3 (e.g., 0, 1, 2, or 3) nucleotides from the nucleotide sequence of any one of the antisense strands set forth in any one of Tables 2-5 or set forth in any one of SEQ ID NOS: 4-1576 (e.g., any one of SEQ ID NOS: 4-19 or 772-787); and the nucleotide sequence of the sense strand differs by no more than 3 (e.g., 0, 1, 2, or 3) nucleotides from the nucleotide sequence of the corresponding sense strand set forth in any one of Tables 2-5 or the corresponding sense strand set forth in one of SEQ ID NOS: 1577-3166 (e.g., any one of SEQ ID NOS: 1577-1592 or 2346-2361).

In some embodiments, the nucleotide sequence of the antisense strand comprises the nucleotide sequence of any one of the antisense strands set forth in any one of Tables 2-5 or set forth in any one of SEQ ID NOS: 4-1576 (e.g., any one of SEQ ID NOS: 4-19 or 772-787); and the nucleotide sequence of the sense strand comprises the nucleotide sequence of the corresponding sense strand set forth in any one of Tables 2-5 or the corresponding sense strand set forth in one of SEQ ID NOS: 1577-3166 (e.g., any one of SEQ ID NOS: 1577-1592 or 2346-2361).

In some embodiments, (a) the nucleotide sequence of the antisense strand differs by no more than 5 (e.g., 0, 1, 2, 3, 4, or 5) nucleotides from the nucleotide sequence set forth in SEQ ID NO: 4; and the nucleotide sequence of the sense strand differs by no more than 5 (e.g., 0, 1, 2, 3, 4, or 5) nucleotides from the nucleotide sequence set forth in SEQ ID NO: 1577; (b) the nucleotide sequence of the antisense strand differs by no more than 5 (e.g., 0, 1, 2, 3, 4, or 5) nucleotides from the nucleotide sequence set forth in SEQ ID NO: 5; and the nucleotide sequence of the sense strand differs by no more than 5 (e.g., 0, 1, 2, 3, 4, or 5) nucleotides from the nucleotide sequence set forth in SEQ ID NO: 1578; (c) the nucleotide sequence of the antisense strand differs by no more than 5 (e.g., 0, 1, 2, 3, 4, or 5) nucleotides from the nucleotide sequence set forth in SEQ ID NO: 6; and the nucleotide sequence of the sense strand differs by no more than 5 (e.g., 0, 1, 2, 3, 4, or 5) nucleotides from the nucleotide sequence set forth in SEQ ID NO: 1579; (d) the nucleotide sequence of the antisense strand differs by no more than 5 (e.g., 0, 1, 2, 3, 4, or 5) nucleotides from the nucleotide sequence set forth in SEQ ID NO: 7; and the nucleotide sequence of the sense strand differs by no more than 5 (e.g., 0, 1, 2, 3, 4, or 5) nucleotides from the nucleotide sequence set forth in SEQ ID NO: 1580; (e) the nucleotide sequence of the antisense strand differs by no more than 5 (e.g., 0, 1, 2, 3, 4, or 5) nucleotides from the nucleotide sequence set forth in SEQ ID NO: 8; and the nucleotide sequence of the sense strand differs by no more than 5 (e.g., 0, 1, 2, 3, 4, or 5) nucleotides from the nucleotide sequence set forth in SEQ ID NO: 1581; (f) the nucleotide sequence of the antisense strand differs by no more than 5 (e.g., 0, 1, 2, 3, 4, or 5) nucleotides from the nucleotide sequence set forth in SEQ ID NO: 9; and the nucleotide sequence of the sense strand differs by no more than 5 (e.g., 0, 1, 2, 3, 4, or 5) nucleotides from the nucleotide sequence set forth in SEQ ID NO: 1582; (g) the nucleotide sequence of the antisense strand differs by no more than 5 (e.g., 0, 1, 2, 3, 4, or 5) nucleotides from the nucleotide sequence set forth in SEQ ID NO: 10; and the nucleotide sequence of the sense strand differs by no more than 5 (e.g., 0, 1, 2, 3, 4, or 5) nucleotides from the nucleotide sequence set forth in SEQ ID NO: 1583; (h) the nucleotide sequence of the antisense strand differs by no more than 5 (e.g., 0, 1, 2, 3, 4, or 5) nucleotides from the nucleotide sequence set forth in SEQ ID NO: 11; and the nucleotide sequence of the sense strand differs by no more than 5 (e.g., 0, 1, 2, 3, 4, or 5) nucleotides from the nucleotide sequence set forth in SEQ ID NO: 1584; (i) the nucleotide sequence of the antisense strand differs by no more than 5 (e.g., 0, 1, 2, 3, 4, or 5) nucleotides from the nucleotide sequence set forth in SEQ ID NO: 12; and the nucleotide sequence of the sense strand differs by no more than 5 (e.g., 0, 1, 2, 3, 4, or 5) nucleotides from the nucleotide sequence set forth in SEQ ID NO: 1585; (j) the nucleotide sequence of the antisense strand differs by no more than 5 (e.g., 0, 1, 2, 3, 4, or 5) nucleotides from the nucleotide sequence set forth in SEQ ID NO: 13; and the nucleotide sequence of the sense strand differs by no more than 5 (e.g., 0, 1, 2, 3, 4, or 5) nucleotides from the nucleotide sequence set forth in SEQ ID NO: 1586; (k) the nucleotide sequence of the antisense strand differs by no more than 5 (e.g., 0, 1, 2, 3, 4, or 5) nucleotides from the nucleotide sequence set forth in SEQ ID NO: 14; and the nucleotide sequence of the sense strand differs by no more than 5 (e.g., 0, 1, 2, 3, 4, or 5) nucleotides from the nucleotide sequence set forth in SEQ ID NO: 1587; (l) the nucleotide sequence of the antisense strand differs by no more than 5 (e.g., 0, 1, 2, 3, 4, or 5) nucleotides from the nucleotide sequence set forth in SEQ ID NO: 15; and the nucleotide sequence of the sense strand differs by no more than 5 (e.g., 0, 1, 2, 3, 4, or 5) nucleotides from the nucleotide sequence set forth in SEQ ID NO: 1588; (m) the nucleotide sequence of the antisense strand differs by no more than 5 (e.g., 0, 1, 2, 3, 4, or 5) nucleotides from the nucleotide sequence set forth in SEQ ID NO: 16; and the nucleotide sequence of the sense strand differs by no more than 5 (e.g., 0, 1, 2, 3, 4, or 5) nucleotides from the nucleotide sequence set forth in SEQ ID NO: 1589; (n) the nucleotide sequence of the antisense strand differs by no more than 5 (e.g., 0, 1, 2, 3, 4, or 5) nucleotides from the nucleotide sequence set forth in SEQ ID NO: 17; and the nucleotide sequence of the sense strand differs by no more than 5 (e.g., 0, 1, 2, 3, 4, or 5) nucleotides from the nucleotide sequence set forth in SEQ ID NO: 1590; (o) the nucleotide sequence of the antisense strand differs by no more than 5 (e.g., 0, 1, 2, 3, 4, or 5) nucleotides from the nucleotide sequence set forth in SEQ ID NO: 18; and the nucleotide sequence of the sense strand differs by no more than 5 (e.g., 0, 1, 2, 3, 4, or 5) nucleotides from the nucleotide sequence set forth in SEQ ID NO: 1591; or (p) the nucleotide sequence of the antisense strand differs by no more than 5 (e.g., 0, 1, 2, 3, 4, or 5) nucleotides from the nucleotide sequence set forth in SEQ ID NO: 19; and the nucleotide sequence of the sense strand differs by no more than 5 (e.g., 0, 1, 2, 3, 4, or 5) nucleotides from the nucleotide sequence set forth in SEQ ID NO: 1592.

In some embodiments, (a) the nucleotide sequence of the antisense strand comprises the nucleotide sequence set forth in SEQ ID NO: 4; and the nucleotide sequence of the sense strand comprises the nucleotide sequence set forth in SEQ ID NO: 1577; (b) the nucleotide sequence of the antisense strand comprises the nucleotide sequence set forth in SEQ ID NO: 5; and the nucleotide sequence of the sense strand comprises the nucleotide sequence set forth in SEQ ID NO: 1578; (c) the nucleotide sequence of the antisense strand comprises the nucleotide sequence set forth in SEQ ID NO: 6; and the nucleotide sequence of the sense strand comprises the nucleotide sequence set forth in SEQ ID NO: 1579; (d) the nucleotide sequence of the antisense strand comprises the nucleotide sequence set forth in SEQ ID NO: 7; and the nucleotide sequence of the sense strand comprises the nucleotide sequence set forth in SEQ ID NO: 1580; (e) the nucleotide sequence of the antisense strand comprises the nucleotide sequence set forth in SEQ ID NO: 8; and the nucleotide sequence of the sense strand comprises the nucleotide sequence set forth in SEQ ID NO: 1581; (f) the nucleotide sequence of the antisense strand comprises the nucleotide sequence set forth in SEQ ID NO: 9; and the nucleotide sequence of the sense strand comprises the nucleotide sequence set forth in SEQ ID NO: 1582; (g) the nucleotide sequence of the antisense strand comprises the nucleotide sequence set forth in SEQ ID NO: 10; and the nucleotide sequence of the sense strand comprises the nucleotide sequence set forth in SEQ ID NO: 1583; (h) the nucleotide sequence of the antisense strand comprises the nucleotide sequence set forth in SEQ ID NO: 11; and the nucleotide sequence of the sense strand comprises the nucleotide sequence set forth in SEQ ID NO: 1584; (i) the nucleotide sequence of the antisense strand comprises the nucleotide sequence set forth in SEQ ID NO: 12; and the nucleotide sequence of the sense strand comprises the nucleotide sequence set forth in SEQ ID NO: 1585; (j) the nucleotide sequence of the antisense strand comprises the nucleotide sequence set forth in SEQ ID NO: 13; and the nucleotide sequence of the sense strand comprises the nucleotide sequence set forth in SEQ ID NO: 1586; (k) the nucleotide sequence of the antisense strand comprises the nucleotide sequence set forth in SEQ ID NO: 14; and the nucleotide sequence of the sense strand comprises the nucleotide sequence set forth in SEQ ID NO: 1587; (l) the nucleotide sequence of the antisense strand comprises the nucleotide sequence set forth in SEQ ID NO: 15; and the nucleotide sequence of the sense strand comprises the nucleotide sequence set forth in SEQ ID NO: 1588; (m) the nucleotide sequence of the antisense strand comprises the nucleotide sequence set forth in SEQ ID NO: 16; and the nucleotide sequence of the sense strand comprises the nucleotide sequence set forth in SEQ ID NO: 1589; (n) the nucleotide sequence of the antisense strand comprises the nucleotide sequence set forth in SEQ ID NO: 17; and the nucleotide sequence of the sense strand comprises the nucleotide sequence set forth in SEQ ID NO: 1590; (o) the nucleotide sequence of the antisense strand comprises the nucleotide sequence set forth in SEQ ID NO: 18; and the nucleotide sequence of the sense strand comprises the nucleotide sequence set forth in SEQ ID NO: 1591; or (p) the nucleotide sequence of the antisense strand comprises the nucleotide sequence set forth in SEQ ID NO: 19; and the nucleotide sequence of the sense strand comprises the nucleotide sequence set forth in SEQ ID NO: 1592.

In some embodiments, (a) the nucleotide sequence of the antisense strand differs by no more than 5 (e.g., 0, 1, 2, 3, 4, or 5) nucleotides from the nucleotide sequence set forth in SEQ ID NO: 772; and the nucleotide sequence of the sense strand differs by no more than 5 (e.g., 0, 1, 2, 3, 4, or 5) nucleotides from the nucleotide sequence set forth in SEQ ID NO: 2346; (b) the nucleotide sequence of the antisense strand differs by no more than 5 (e.g., 0, 1, 2, 3, 4, or 5) nucleotides from the nucleotide sequence set forth in SEQ ID NO: 773; and the nucleotide sequence of the sense strand differs by no more than 5 (e.g., 0, 1, 2, 3, 4, or 5) nucleotides from the nucleotide sequence set forth in SEQ ID NO: 2347; (c) the nucleotide sequence of the antisense strand differs by no more than 5 (e.g., 0, 1, 2, 3, 4, or 5) nucleotides from the nucleotide sequence set forth in SEQ ID NO: 774; and the nucleotide sequence of the sense strand differs by no more than 5 (e.g., 0, 1, 2, 3, 4, or 5) nucleotides from the nucleotide sequence set forth in SEQ ID NO: 2348; (d) the nucleotide sequence of the antisense strand differs by no more than 5 (e.g., 0, 1, 2, 3, 4, or 5) nucleotides from the nucleotide sequence set forth in SEQ ID NO: 775; and the nucleotide sequence of the sense strand differs by no more than 5 (e.g., 0, 1, 2, 3, 4, or 5) nucleotides from the nucleotide sequence set forth in SEQ ID NO: 2349; (e) the nucleotide sequence of the antisense strand differs by no more than 5 (e.g., 0, 1, 2, 3, 4, or 5) nucleotides from the nucleotide sequence set forth in SEQ ID NO: 776; and the nucleotide sequence of the sense strand differs by no more than 5 (e.g., 0, 1, 2, 3, 4, or 5) nucleotides from the nucleotide sequence set forth in SEQ ID NO: 2350; (f) the nucleotide sequence of the antisense strand differs by no more than 5 (e.g., 0, 1, 2, 3, 4, or 5) nucleotides from the nucleotide sequence set forth in SEQ ID NO: 777; and the nucleotide sequence of the sense strand differs by no more than 5 (e.g., 0, 1, 2, 3, 4, or 5) nucleotides from the nucleotide sequence set forth in SEQ ID NO: 2351; (g) the nucleotide sequence of the antisense strand differs by no more than 5 (e.g., 0, 1, 2, 3, 4, or 5) nucleotides from the nucleotide sequence set forth in SEQ ID NO: 778; and the nucleotide sequence of the sense strand differs by no more than 5 (e.g., 0, 1, 2, 3, 4, or 5) nucleotides from the nucleotide sequence set forth in SEQ ID NO: 2352; (h) the nucleotide sequence of the antisense strand differs by no more than 5 (e.g., 0, 1, 2, 3, 4, or 5) nucleotides from the nucleotide sequence set forth in SEQ ID NO: 779; and the nucleotide sequence of the sense strand differs by no more than 5 (e.g., 0, 1, 2, 3, 4, or 5) nucleotides from the nucleotide sequence set forth in SEQ ID NO: 2353; (i) the nucleotide sequence of the antisense strand differs by no more than 5 (e.g., 0, 1, 2, 3, 4, or 5) nucleotides from the nucleotide sequence set forth in SEQ ID NO: 780; and the nucleotide sequence of the sense strand differs by no more than 5 (e.g., 0, 1, 2, 3, 4, or 5) nucleotides from the nucleotide sequence set forth in SEQ ID NO: 2354; (j) the nucleotide sequence of the antisense strand differs by no more than 5 (e.g., 0, 1, 2, 3, 4, or 5) nucleotides from the nucleotide sequence set forth in SEQ ID NO: 781; and the nucleotide sequence of the sense strand differs by no more than 5 (e.g., 0, 1, 2, 3, 4, or 5) nucleotides from the nucleotide sequence set forth in SEQ ID NO: 2355; (k) the nucleotide sequence of the antisense strand differs by no more than 5 (e.g., 0, 1, 2, 3, 4, or 5) nucleotides from the nucleotide sequence set forth in SEQ ID NO: 782; and the nucleotide sequence of the sense strand differs by no more than 5 (e.g., 0, 1, 2, 3, 4, or 5) nucleotides from the nucleotide sequence set forth in SEQ ID NO: 2356; (l) the nucleotide sequence of the antisense strand differs by no more than 5 (e.g., 0, 1, 2, 3, 4, or 5) nucleotides from the nucleotide sequence set forth in SEQ ID NO: 783; and the nucleotide sequence of the sense strand differs by no more than 5 (e.g., 0, 1, 2, 3, 4, or 5) nucleotides from the nucleotide sequence set forth in SEQ ID NO: 2357; (m) the nucleotide sequence of the antisense strand differs by no more than 5 (e.g., 0, 1, 2, 3, 4, or 5) nucleotides from the nucleotide sequence set forth in SEQ ID NO: 784; and the nucleotide sequence of the sense strand differs by no more than 5 (e.g., 0, 1, 2, 3, 4, or 5) nucleotides from the nucleotide sequence set forth in SEQ ID NO: 2358; (n) the nucleotide sequence of the antisense strand differs by no more than 5 (e.g., 0, 1, 2, 3, 4, or 5) nucleotides from the nucleotide sequence set forth in SEQ ID NO: 785; and the nucleotide sequence of the sense strand differs by no more than 5 (e.g., 0, 1, 2, 3, 4, or 5) nucleotides from the nucleotide sequence set forth in SEQ ID NO: 2359; (o) the nucleotide sequence of the antisense strand differs by no more than 5 (e.g., 0, 1, 2, 3, 4, or 5) nucleotides from the nucleotide sequence set forth in SEQ ID NO: 786; and the nucleotide sequence of the sense strand differs by no more than 5 (e.g., 0, 1, 2, 3, 4, or 5) nucleotides from the nucleotide sequence set forth in SEQ ID NO: 2360; or (p) the nucleotide sequence of the antisense strand differs by no more than 5 (e.g., 0, 1, 2, 3, 4, or 5) nucleotides from the nucleotide sequence set forth in SEQ ID NO: 787; and the nucleotide sequence of the sense strand differs by no more than 5 (e.g., 0, 1, 2, 3, 4, or 5) nucleotides from the nucleotide sequence set forth in SEQ ID NO: 2361.

In some embodiments, (a) the nucleotide sequence of the antisense strand comprises the nucleotide sequence set forth in SEQ ID NO: 772; and the nucleotide sequence of the sense strand comprises the nucleotide sequence set forth in SEQ ID NO: 2346; (b) the nucleotide sequence of the antisense strand comprises the nucleotide sequence set forth in SEQ ID NO: 773; and the nucleotide sequence of the sense strand comprises the nucleotide sequence set forth in SEQ ID NO: 2347; (c) the nucleotide sequence of the antisense strand comprises the nucleotide sequence set forth in SEQ ID NO: 774; and the nucleotide sequence of the sense strand comprises the nucleotide sequence set forth in SEQ ID NO: 2348; (d) the nucleotide sequence of the antisense strand comprises the nucleotide sequence set forth in SEQ ID NO: 775; and the nucleotide sequence of the sense strand comprises the nucleotide sequence set forth in SEQ ID NO: 2349; (e) the nucleotide sequence of the antisense strand comprises the nucleotide sequence set forth in SEQ ID NO: 776; and the nucleotide sequence of the sense strand comprises the nucleotide sequence set forth in SEQ ID NO: 2350; (f) the nucleotide sequence of the antisense strand comprises the nucleotide sequence set forth in SEQ ID NO: 777; and the nucleotide sequence of the sense strand comprises the nucleotide sequence set forth in SEQ ID NO: 2351; (g) the nucleotide sequence of the antisense strand comprises the nucleotide sequence set forth in SEQ ID NO: 778; and the nucleotide sequence of the sense strand comprises the nucleotide sequence set forth in SEQ ID NO: 2352; (h) the nucleotide sequence of the antisense strand comprises the nucleotide sequence set forth in SEQ ID NO: 779; and the nucleotide sequence of the sense strand comprises the nucleotide sequence set forth in SEQ ID NO: 2353; (i) the nucleotide sequence of the antisense strand comprises the nucleotide sequence set forth in SEQ ID NO: 780; and the nucleotide sequence of the sense strand comprises the nucleotide sequence set forth in SEQ ID NO: 2354; (j) the nucleotide sequence of the antisense strand comprises the nucleotide sequence set forth in SEQ ID NO: 781; and the nucleotide sequence of the sense strand comprises the nucleotide sequence set forth in SEQ ID NO: 2355; (k) the nucleotide sequence of the antisense strand comprises the nucleotide sequence set forth in SEQ ID NO: 782; and the nucleotide sequence of the sense strand comprises the nucleotide sequence set forth in SEQ ID NO: 2356; (l) the nucleotide sequence of the antisense strand comprises the nucleotide sequence set forth in SEQ ID NO: 783; and the nucleotide sequence of the sense strand comprises the nucleotide sequence set forth in SEQ ID NO: 2357; (m) the nucleotide sequence of the antisense strand comprises the nucleotide sequence set forth in SEQ ID NO: 784; and the nucleotide sequence of the sense strand comprises the nucleotide sequence set forth in SEQ ID NO: 2358; (n) the nucleotide sequence of the antisense strand comprises the nucleotide sequence set forth in SEQ ID NO: 785; and the nucleotide sequence of the sense strand comprises the nucleotide sequence set forth in SEQ ID NO: 2359; (o) the nucleotide sequence of the antisense strand comprises the nucleotide sequence set forth in SEQ ID NO: 786; and the nucleotide sequence of the sense strand comprises the nucleotide sequence set forth in SEQ ID NO: 2360; or (p) the nucleotide sequence of the antisense strand comprises the nucleotide sequence set forth in SEQ ID NO: 787; and the nucleotide sequence of the sense strand comprises the nucleotide sequence set forth in SEQ ID NO: 2361.

In one aspect, provided herein are dsRNA agents for inhibiting expression of BCL11A (e.g., hBCL11A), wherein the dsRNA agent comprises a sense strand and an antisense strand forming a double stranded region, and wherein the nucleotide sequence of the antisense strand differs by no more than 5 (e.g., 0, 1, 2, 3, 4, or 5) nucleotides from the nucleotide sequence of any one of the antisense strands of any one of dsRNA agents 1-1590 (e.g., one of dsRNA agents 1-16 or 770-785) set forth in Tables 2-5; and the nucleotide sequence of the sense strand differs by no more than 5 (e.g., 0, 1, 2, 3, 4, or 5) nucleotides from the nucleotide sequence of the sense strand of the corresponding dsRNA agent set forth in Tables 2-5.

In some embodiments, the nucleotide sequence of the antisense strand differs by no more than 3 (e.g., 0, 1, 2, or 3) nucleotides from the nucleotide sequence of any one of the antisense strands of any one of dsRNA agents 1-1590 (e.g., one of dsRNA agents 1-16 or 770-785) set forth in Tables 2-5; and the nucleotide sequence of the sense strand differs by no more than 3 (e.g., 0, 1, 2, or 3) nucleotides from the nucleotide sequence of the sense strand of the corresponding dsRNA agent set forth in Tables 2-5.

In some embodiments, the nucleotide sequence of the antisense strand differs by no more than 3 (e.g., 0, 1, 2, or 3) nucleotides from the nucleotide sequence of any one of the antisense strands of any one of dsRNA agents 1-16 or 770-785 set forth in Tables 2-5; and the nucleotide sequence of the sense strand differs by no more than 3 (e.g., 0, 1, 2, or 3) nucleotides from the nucleotide sequence of the sense strand of the corresponding dsRNA agent set forth in Tables 2-5.

In some embodiments, (a) the nucleotide sequence of the antisense strand differs by no more than 5 (e.g., 0, 1, 2, 3, 4, or 5) nucleotides from the nucleotide sequence of the antisense strand of dsRNA agent 1; and the nucleotide sequence of the sense strand differs by no more than 5 (e.g., 0, 1, 2, 3, 4, or 5) nucleotides from the nucleotide sequence of dsRNA agent 1; (b) the nucleotide sequence of the antisense strand differs by no more than 5 (e.g., 0, 1, 2, 3, 4, or 5) nucleotides from the nucleotide sequence of the antisense strand of dsRNA agent 2; and the nucleotide sequence of the sense strand differs by no more than 5 (e.g., 0, 1, 2, 3, 4, or 5) nucleotides from the nucleotide sequence of dsRNA agent 2; (c) the nucleotide sequence of the antisense strand differs by no more than 5 (e.g., 0, 1, 2, 3, 4, or 5) nucleotides from the nucleotide sequence of the antisense strand of dsRNA agent 3; and the nucleotide sequence of the sense strand differs by no more than 5 (e.g., 0, 1, 2, 3, 4, or 5) nucleotides from the nucleotide sequence of dsRNA agent 3; (d) the nucleotide sequence of the antisense strand differs by no more than 5 (e.g., 0, 1, 2, 3, 4, or 5) nucleotides from the nucleotide sequence of the antisense strand of dsRNA agent 4; and the nucleotide sequence of the sense strand differs by no more than 5 (e.g., 0, 1, 2, 3, 4, or 5) nucleotides from the nucleotide sequence of dsRNA agent 4; (e) the nucleotide sequence of the antisense strand differs by no more than 5 (e.g., 0, 1, 2, 3, 4, or 5) nucleotides from the nucleotide sequence of the antisense strand of dsRNA agent 5; and the nucleotide sequence of the sense strand differs by no more than 5 (e.g., 0, 1, 2, 3, 4, or 5) nucleotides from the nucleotide sequence of dsRNA agent 5; (f) the nucleotide sequence of the antisense strand differs by no more than 5 (e.g., 0, 1, 2, 3, 4, or 5) nucleotides from the nucleotide sequence of the antisense strand of dsRNA agent 6; and the nucleotide sequence of the sense strand differs by no more than 5 (e.g., 0, 1, 2, 3, 4, or 5) nucleotides from the nucleotide sequence of dsRNA agent 6; (g) the nucleotide sequence of the antisense strand differs by no more than 5 (e.g., 0, 1, 2, 3, 4, or 5) nucleotides from the nucleotide sequence of the antisense strand of dsRNA agent 7; and the nucleotide sequence of the sense strand differs by no more than 5 (e.g., 0, 1, 2, 3, 4, or 5) nucleotides from the nucleotide sequence of dsRNA agent 7; (h) the nucleotide sequence of the antisense strand differs by no more than 5 (e.g., 0, 1, 2, 3, 4, or 5) nucleotides from the nucleotide sequence of the antisense strand of dsRNA agent 8; and the nucleotide sequence of the sense strand differs by no more than 5 (e.g., 0, 1, 2, 3, 4, or 5) nucleotides from the nucleotide sequence of dsRNA agent 8; (i) the nucleotide sequence of the antisense strand differs by no more than 5 (e.g., 0, 1, 2, 3, 4, or 5) nucleotides from the nucleotide sequence of the antisense strand of dsRNA agent 9; and the nucleotide sequence of the sense strand differs by no more than 5 (e.g., 0, 1, 2, 3, 4, or 5) nucleotides from the nucleotide sequence of dsRNA agent 9; (j) the nucleotide sequence of the antisense strand differs by no more than 5 (e.g., 0, 1, 2, 3, 4, or 5) nucleotides from the nucleotide sequence of the antisense strand of dsRNA agent 10; and the nucleotide sequence of the sense strand differs by no more than 5 (e.g., 0, 1, 2, 3, 4, or 5) nucleotides from the nucleotide sequence of dsRNA agent 10; (k) the nucleotide sequence of the antisense strand differs by no more than 5 (e.g., 0, 1, 2, 3, 4, or 5) nucleotides from the nucleotide sequence of the antisense strand of dsRNA agent 11; and the nucleotide sequence of the sense strand differs by no more than 5 (e.g., 0, 1, 2, 3, 4, or 5) nucleotides from the nucleotide sequence of dsRNA agent 11; (l) the nucleotide sequence of the antisense strand differs by no more than 5 (e.g., 0, 1, 2, 3, 4, or 5) nucleotides from the nucleotide sequence of the antisense strand of dsRNA agent 12; and the nucleotide sequence of the sense strand differs by no more than 5 (e.g., 0, 1, 2, 3, 4, or 5) nucleotides from the nucleotide sequence of dsRNA agent 12; (m) the nucleotide sequence of the antisense strand differs by no more than 5 (e.g., 0, 1, 2, 3, 4, or 5) nucleotides from the nucleotide sequence of the antisense strand of dsRNA agent 13; and the nucleotide sequence of the sense strand differs by no more than 5 (e.g., 0, 1, 2, 3, 4, or 5) nucleotides from the nucleotide sequence of dsRNA agent 13; (n) the nucleotide sequence of the antisense strand differs by no more than 5 (e.g., 0, 1, 2, 3, 4, or 5) nucleotides from the nucleotide sequence of the antisense strand of dsRNA agent 14; and the nucleotide sequence of the sense strand differs by no more than 5 (e.g., 0, 1, 2, 3, 4, or 5) nucleotides from the nucleotide sequence of dsRNA agent 14; (o) the nucleotide sequence of the antisense strand differs by no more than 5 (e.g., 0, 1, 2, 3, 4, or 5) nucleotides from the nucleotide sequence of the antisense strand of dsRNA agent 15; and the nucleotide sequence of the sense strand differs by no more than 5 (e.g., 0, 1, 2, 3, 4, or 5) nucleotides from the nucleotide sequence of dsRNA agent 15; or (p) the nucleotide sequence of the antisense strand differs by no more than 5 (e.g., 0, 1, 2, 3, 4, or 5) nucleotides from the nucleotide sequence of the antisense strand of dsRNA agent 16; and the nucleotide sequence of the sense strand differs by no more than 5 (e.g., 0, 1, 2, 3, 4, or 5) nucleotides from the nucleotide sequence of dsRNA agent 16.

In some embodiments, (a) the nucleotide sequence of the antisense strand comprises the nucleotide sequence of the antisense strand of dsRNA agent 1; and the nucleotide sequence of the sense strand comprises of the sense strand of dsRNA agent 1; (b) the nucleotide sequence of the antisense strand comprises the nucleotide sequence of the antisense strand of dsRNA agent 2; and the nucleotide sequence of the sense strand comprises of the sense strand of dsRNA agent 2; (c) the nucleotide sequence of the antisense strand comprises the nucleotide sequence of the antisense strand of dsRNA agent 3; and the nucleotide sequence of the sense strand comprises of the sense strand of dsRNA agent 3; (d) the nucleotide sequence of the antisense strand comprises the nucleotide sequence of the antisense strand of dsRNA agent 4; and the nucleotide sequence of the sense strand comprises of the sense strand of dsRNA agent 4; (e) the nucleotide sequence of the antisense strand comprises the nucleotide sequence of the antisense strand of dsRNA agent 5; and the nucleotide sequence of the sense strand comprises of the sense strand of dsRNA agent 5; (f) the nucleotide sequence of the antisense strand comprises the nucleotide sequence of the antisense strand of dsRNA agent 6; and the nucleotide sequence of the sense strand comprises of the sense strand of dsRNA agent 6; (g) the nucleotide sequence of the antisense strand comprises the nucleotide sequence of the antisense strand of dsRNA agent 7; and the nucleotide sequence of the sense strand comprises of the sense strand of dsRNA agent 7; (h) the nucleotide sequence of the antisense strand comprises the nucleotide sequence of the antisense strand of dsRNA agent 8; and the nucleotide sequence of the sense strand comprises of the sense strand of dsRNA agent 8; (i) the nucleotide sequence of the antisense strand comprises the nucleotide sequence of the antisense strand of dsRNA agent 9; and the nucleotide sequence of the sense strand comprises of the sense strand of dsRNA agent 9; (j) the nucleotide sequence of the antisense strand comprises the nucleotide sequence of the antisense strand of dsRNA agent 10; and the nucleotide sequence of the sense strand comprises of the sense strand of dsRNA agent 10; (k) the nucleotide sequence of the antisense strand comprises the nucleotide sequence of the antisense strand of dsRNA agent 11; and the nucleotide sequence of the sense strand comprises of the sense strand of dsRNA agent 11; (l) the nucleotide sequence of the antisense strand comprises the nucleotide sequence of the antisense strand of dsRNA agent 12; and the nucleotide sequence of the sense strand comprises of the sense strand of dsRNA agent 12; (m) the nucleotide sequence of the antisense strand comprises the nucleotide sequence of the antisense strand of dsRNA agent 13; and the nucleotide sequence of the sense strand comprises of the sense strand of dsRNA agent 13; (n) the nucleotide sequence of the antisense strand comprises the nucleotide sequence of the antisense strand of dsRNA agent 14; and the nucleotide sequence of the sense strand comprises of the sense strand of dsRNA agent 14; (o) the nucleotide sequence of the antisense strand comprises the nucleotide sequence of the antisense strand of dsRNA agent 15; and the nucleotide sequence of the sense strand comprises of the sense strand of dsRNA agent 15; or (p) the nucleotide sequence of the antisense strand comprises the nucleotide sequence of the antisense strand of dsRNA agent 16; and the nucleotide sequence of the sense strand comprises of the sense strand of dsRNA agent 16.

In some embodiments, (a) the nucleotide sequence of the antisense strand differs by no more than 5 (e.g., 0, 1, 2, 3, 4, or 5) nucleotides from the nucleotide sequence of the antisense strand of dsRNA agent 770; and the nucleotide sequence of the sense strand differs by no more than 5 (e.g., 0, 1, 2, 3, 4, or 5) nucleotides from the nucleotide sequence of dsRNA agent 770; (b) the nucleotide sequence of the antisense strand differs by no more than 5 (e.g., 0, 1, 2, 3, 4, or 5) nucleotides from the nucleotide sequence of the antisense strand of dsRNA agent 771; and the nucleotide sequence of the sense strand differs by no more than 5 (e.g., 0, 1, 2, 3, 4, or 5) nucleotides from the nucleotide sequence of dsRNA agent 771; (c) the nucleotide sequence of the antisense strand differs by no more than 5 (e.g., 0, 1, 2, 3, 4, or 5) nucleotides from the nucleotide sequence of the antisense strand of dsRNA agent 772; and the nucleotide sequence of the sense strand differs by no more than 5 (e.g., 0, 1, 2, 3, 4, or 5) nucleotides from the nucleotide sequence of dsRNA agent 772; (d) the nucleotide sequence of the antisense strand differs by no more than 5 (e.g., 0, 1, 2, 3, 4, or 5) nucleotides from the nucleotide sequence of the antisense strand of dsRNA agent 773; and the nucleotide sequence of the sense strand differs by no more than 5 (e.g., 0, 1, 2, 3, 4, or 5) nucleotides from the nucleotide sequence of dsRNA agent 773; (e) the nucleotide sequence of the antisense strand differs by no more than 5 (e.g., 0, 1, 2, 3, 4, or 5) nucleotides from the nucleotide sequence of the antisense strand of dsRNA agent 774; and the nucleotide sequence of the sense strand differs by no more than 5 (e.g., 0, 1, 2, 3, 4, or 5) nucleotides from the nucleotide sequence of dsRNA agent 774; (f) the nucleotide sequence of the antisense strand differs by no more than 5 (e.g., 0, 1, 2, 3, 4, or 5) nucleotides from the nucleotide sequence of the antisense strand of dsRNA agent 775; and the nucleotide sequence of the sense strand differs by no more than 5 (e.g., 0, 1, 2, 3, 4, or 5) nucleotides from the nucleotide sequence of dsRNA agent 775; (g) the nucleotide sequence of the antisense strand differs by no more than 5 (e.g., 0, 1, 2, 3, 4, or 5) nucleotides from the nucleotide sequence of the antisense strand of dsRNA agent 776; and the nucleotide sequence of the sense strand differs by no more than 5 (e.g., 0, 1, 2, 3, 4, or 5) nucleotides from the nucleotide sequence of dsRNA agent 776; (h) the nucleotide sequence of the antisense strand differs by no more than 5 (e.g., 0, 1, 2, 3, 4, or 5) nucleotides from the nucleotide sequence of the antisense strand of dsRNA agent 777; and the nucleotide sequence of the sense strand differs by no more than 5 (e.g., 0, 1, 2, 3, 4, or 5) nucleotides from the nucleotide sequence of dsRNA agent 778; (i) the nucleotide sequence of the antisense strand differs by no more than 5 (e.g., 0, 1, 2, 3, 4, or 5) nucleotides from the nucleotide sequence of the antisense strand of dsRNA agent 779; and the nucleotide sequence of the sense strand differs by no more than 5 (e.g., 0, 1, 2, 3, 4, or 5) nucleotides from the nucleotide sequence of dsRNA agent 779; (j) the nucleotide sequence of the antisense strand differs by no more than 5 (e.g., 0, 1, 2, 3, 4, or 5) nucleotides from the nucleotide sequence of the antisense strand of dsRNA agent 780; and the nucleotide sequence of the sense strand differs by no more than 5 (e.g., 0, 1, 2, 3, 4, or 5) nucleotides from the nucleotide sequence of dsRNA agent 780; (k) the nucleotide sequence of the antisense strand differs by no more than 5 (e.g., 0, 1, 2, 3, 4, or 5) nucleotides from the nucleotide sequence of the antisense strand of dsRNA agent 781; and the nucleotide sequence of the sense strand differs by no more than 5 (e.g., 0, 1, 2, 3, 4, or 5) nucleotides from the nucleotide sequence of dsRNA agent 781; (l) the nucleotide sequence of the antisense strand differs by no more than 5 (e.g., 0, 1, 2, 3, 4, or 5) nucleotides from the nucleotide sequence of the antisense strand of dsRNA agent 782; and the nucleotide sequence of the sense strand differs by no more than 5 (e.g., 0, 1, 2, 3, 4, or 5) nucleotides from the nucleotide sequence of dsRNA agent 782; (m) the nucleotide sequence of the antisense strand differs by no more than 5 (e.g., 0, 1, 2, 3, 4, or 5) nucleotides from the nucleotide sequence of the antisense strand of dsRNA agent 783; and the nucleotide sequence of the sense strand differs by no more than 5 (e.g., 0, 1, 2, 3, 4, or 5) nucleotides from the nucleotide sequence of dsRNA agent 783; (n) the nucleotide sequence of the antisense strand differs by no more than 5 (e.g., 0, 1, 2, 3, 4, or 5) nucleotides from the nucleotide sequence of the antisense strand of dsRNA agent 14; and the nucleotide sequence of the sense strand differs by no more than 5 (e.g., 0, 1, 2, 3, 4, or 5) nucleotides from the nucleotide sequence of dsRNA agent 14; (o) the nucleotide sequence of the antisense strand differs by no more than 5 (e.g., 0, 1, 2, 3, 4, or 5) nucleotides from the nucleotide sequence of the antisense strand of dsRNA agent 784; and the nucleotide sequence of the sense strand differs by no more than 5 (e.g., 0, 1, 2, 3, 4, or 5) nucleotides from the nucleotide sequence of dsRNA agent 784; or (p) the nucleotide sequence of the antisense strand differs by no more than 5 (e.g., 0, 1, 2, 3, 4, or 5) nucleotides from the nucleotide sequence of the antisense strand of dsRNA agent 785; and the nucleotide sequence of the sense strand differs by no more than 5 (e.g., 0, 1, 2, 3, 4, or 5) nucleotides from the nucleotide sequence of dsRNA agent 785.

In some embodiments, (a) the nucleotide sequence of the antisense strand comprises the nucleotide sequence of the antisense strand of dsRNA agent 770; and the nucleotide sequence of the sense strand comprises of the sense strand of dsRNA agent 770; (b) the nucleotide sequence of the antisense strand comprises the nucleotide sequence of the antisense strand of dsRNA agent 771; and the nucleotide sequence of the sense strand comprises of the sense strand of dsRNA agent 771; (c) the nucleotide sequence of the antisense strand comprises the nucleotide sequence of the antisense strand of dsRNA agent 772; and the nucleotide sequence of the sense strand comprises of the sense strand of dsRNA agent 772; (d) the nucleotide sequence of the antisense strand comprises the nucleotide sequence of the antisense strand of dsRNA agent 773; and the nucleotide sequence of the sense strand comprises of the sense strand of dsRNA agent 773; (e) the nucleotide sequence of the antisense strand comprises the nucleotide sequence of the antisense strand of dsRNA agent 774; and the nucleotide sequence of the sense strand comprises of the sense strand of dsRNA agent 774; (f) the nucleotide sequence of the antisense strand comprises the nucleotide sequence of the antisense strand of dsRNA agent 775; and the nucleotide sequence of the sense strand comprises of the sense strand of dsRNA agent 775; (g) the nucleotide sequence of the antisense strand comprises the nucleotide sequence of the antisense strand of dsRNA agent 776; and the nucleotide sequence of the sense strand comprises of the sense strand of dsRNA agent 776; (h) the nucleotide sequence of the antisense strand comprises the nucleotide sequence of the antisense strand of dsRNA agent 777; and the nucleotide sequence of the sense strand comprises of the sense strand of dsRNA agent 777; (i) the nucleotide sequence of the antisense strand comprises the nucleotide sequence of the antisense strand of dsRNA agent 778; and the nucleotide sequence of the sense strand comprises of the sense strand of dsRNA agent 778; (j) the nucleotide sequence of the antisense strand comprises the nucleotide sequence of the antisense strand of dsRNA agent 779; and the nucleotide sequence of the sense strand comprises of the sense strand of dsRNA agent 779; (k) the nucleotide sequence of the antisense strand comprises the nucleotide sequence of the antisense strand of dsRNA agent 780; and the nucleotide sequence of the sense strand comprises of the sense strand of dsRNA agent 780; (l) the nucleotide sequence of the antisense strand comprises the nucleotide sequence of the antisense strand of dsRNA agent 781; and the nucleotide sequence of the sense strand comprises of the sense strand of dsRNA agent 781; (m) the nucleotide sequence of the antisense strand comprises the nucleotide sequence of the antisense strand of dsRNA agent 782; and the nucleotide sequence of the sense strand comprises of the sense strand of dsRNA agent 782; (n) the nucleotide sequence of the antisense strand comprises the nucleotide sequence of the antisense strand of dsRNA agent 783; and the nucleotide sequence of the sense strand comprises of the sense strand of dsRNA agent 783; (o) the nucleotide sequence of the antisense strand comprises the nucleotide sequence of the antisense strand of dsRNA agent 784; and the nucleotide sequence of the sense strand comprises of the sense strand of dsRNA agent 784; or (p) the nucleotide sequence of the antisense strand comprises the nucleotide sequence of the antisense strand of dsRNA agent 785; and the nucleotide sequence of the sense strand comprises of the sense strand of dsRNA agent 785.

In one aspect, provided herein are dsRNA agents for inhibiting expression of BCL11 transcription factor A (BCL11A) (e.g., human BCL11A (hBCL11A)), wherein the dsRNA agent comprises a sense strand and an antisense strand forming a double stranded region, wherein the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of any one of the antisense strands set forth in any one of Tables 2-5 (e.g., Table 3 or 5) or set forth in any one of SEQ ID NOS: 4-1576 (e.g., any one of SEQ ID NOS: 4-19 or 772-787).

In some embodiments, the sense strand comprises at least 15 (e.g., 16, 17, 18, 19, 20, 21) contiguous nucleotides of and differing by no more than 5 (e.g., 0, 1, 2, 3, 4, or 5) nucleotides from any sense strand set forth in any one of Tables 2-5 (e.g., Table 3 or 5) or set forth in any one of SEQ ID NOS: 1577-3166 (e.g., any one of SEQ ID NOS: 1577-1592 or 2346-2361).

In some embodiments, the sense strand comprises at least 15 (e.g., 16, 17, 18, 19, 20, 21) contiguous nucleotides of and differing by no more than 5 (e.g., 0, 1, 2, 3, 4, or 5) nucleotides from the corresponding sense strand set forth in any one of Tables 2-5 (e.g., Table 3 or 5) or the corresponding sense strand set forth in one of SEQ ID NOS: 1577-3166.

In some embodiments, (a) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 4; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1577; (b) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 5; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1578; (c) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 6; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1579; (d) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 7; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1580; (e) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 8; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1581; (f) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 9; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1582; (g) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 10; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1583; (h) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 11; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1584; (i) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 12; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1585; (j) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 13; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1586; (k) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 14; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1587; (l) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 15; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1588; (m) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 16; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1589; (n) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 17; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1590; (o) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 18; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1591; or (p) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 19; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 1592.

In some embodiments, (a) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 772; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2346; (b) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 773; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2347; (c) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 774; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2348; (d) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 775; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2349; (e) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 776; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2350; (f) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 777; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2351; (g) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 778; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2352; (h) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 779; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2353; (i) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 780; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2354; (j) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 781; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2355; (k) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 782; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2356; (l) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 783; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2357; (m) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 784; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2358; (n) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 785; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2359; (o) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 786; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2360; or (p) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of set forth in SEQ ID NO: 787; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence set forth in SEQ ID NO: 2361.

In one aspect, provided herein are dsRNA agents for inhibiting expression of BCL11A (e.g., hBCL11A), wherein the dsRNA agent comprises a sense strand and an antisense strand forming a double stranded region, and wherein the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strands of any one of dsRNA agents 1-1590 (e.g., one of dsRNA agents 1-16 or 770-785) set forth in any one of Tables 2-5 (e.g., Table 3 or 5); and wherein the sense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the sense strand of the corresponding dsRNA agent set forth in any one of Tables 2-5 (e.g., Table 3 or 5).

In some embodiments, the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the corresponding sense strand of the dsRNA agent.

In some embodiments, the nucleotide sequence of the antisense strand comprises at least 15 (e.g., 16, 17, 18, 19, 20, 21, 22, or 23) contiguous nucleotides of and differing by no more than 3 (e.g., 0, 1, 2, or 3) nucleotides from the nucleotide sequence of any one of the antisense strand of any one of dsRNA agents 129, 135, 169, 170, 409, 479, 480, 481, or 482; and wherein the nucleotide sequence of the sense strand comprises at least 15 (e.g., 16, 17, 18, 19, 20, 21) contiguous nucleotides of and differing by no more than 3 (e.g., 0, 1, 2, or 3) nucleotides from the nucleotide sequence of the corresponding sense strand of the dsRNA agent.

In some embodiments, the nucleotide sequence of the antisense strand comprises at least 21 (e.g., 21, 22, or 23) contiguous nucleotides of the nucleotide sequence of any one of the antisense strands of any one of dsRNA agents 1-16 or 770-785; and wherein the nucleotide sequence of the sense strand comprises at least 18 (e.g., 18, 19, 20, 21) contiguous nucleotides of the nucleotide sequence of the corresponding sense strand of the dsRNA agent.

In some embodiments, (a) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 1; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 1; (b) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 2; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 2; (c) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 3; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 3; (d) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 4; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 4; (e) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 5; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 5; (f) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 6; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 6; (g) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 7; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 7; (h) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 8; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 8; (i) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 9; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 9; (j) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 10; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 10; (k) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 11; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 11; (l) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 12; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 12; (m) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 13; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 13; (n) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 14; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 14; (o) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 15; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 15; or (p) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 16; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 16.

In some embodiments, (a) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 770; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 770; (b) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 771; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 771; (c) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 772; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 772; (d) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 773; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 773; (e) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 774; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 774; (f) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 775; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 775; (g) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 776; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 776; (h) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 777; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 777; (i) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 778; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 778; (j) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 779; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 779; (k) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 780; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 780; (l) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 781; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 781; (m) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 782; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 782; (n) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 783; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 783; (o) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 784; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 784; or (p) the nucleotide sequence of the antisense strand comprises at least 15 (e.g., 16, 17, 18, 19, 20, 21, 22, or 23) contiguous nucleotides of and differing by no more than 5 (e.g., 0, 1, 2, 3, 4, or 5) nucleotides from the nucleotide sequence of the antisense strand of dsRNA agent 785; and the nucleotide sequence of the sense strand comprises at least 15 (e.g., 16, 17, 18, 19, 20, 21) contiguous nucleotides of and differing by no more than 5 (e.g., 0, 1, 2, 3, 4, or 5) nucleotides from the nucleotide sequence of the sense strand of dsRNA agent 785.

In one aspect, provided herein are double stranded ribonucleic acid (dsRNA) agents for inhibiting expression of BCL11 transcription factor A (BCL11A) (e.g., human BCL11A (hBCL11A)), wherein the dsRNA agent comprises a sense strand and an antisense strand forming a double stranded region, wherein the antisense strand comprises at least 15 (e.g., 16, 17, 18, 19, 20, 21, 22, or 23) contiguous nucleotides of and differing by no more than 3 (e.g., 0, 1, 2, or 3) nucleotides from the nucleotide sequence of any one of the antisense strands set forth in any one of Tables 2-5 (e.g., Table 3 or 5) or set forth in any one of SEQ ID NOS: 4-1576 (e.g., any one of SEQ ID NOS: 4-19 or 772-787).

In some embodiments, the sense strand comprises at least 15 (e.g., 16, 17, 18, 19, 20, 21) contiguous nucleotides of and differing by no more than 3 (e.g., 0, 1, 2, or 3) nucleotides from any sense strand set forth in any one of Tables 2-5 (e.g., Table 3 or 5) or set forth in any one of SEQ ID NOS: 1577-3166 (e.g., any one of SEQ ID NOS: 1577-1592 or 2346-2361).

In some embodiments, the sense strand comprises at least 15 (e.g., 16, 17, 18, 19, 20, 21) contiguous nucleotides of and differing by no more than 3 (e.g., 0, 1, 2, or 3) nucleotides from the corresponding sense strand set forth in any one of Tables 2-5 (e.g., Table 3 or 5) or the corresponding sense strand set forth in one of SEQ ID NOS: 1577-3166.

In one aspect, provided herein are dsRNA agents for inhibiting expression of BCL11A (e.g., hBCL11A), wherein the dsRNA agent comprises a sense strand and an antisense strand forming a double stranded region, and wherein the antisense strand comprises at least 15 (e.g., 16, 17, 18, 19, 20, 21, 22, or 23) contiguous nucleotides of and differing by no more than 3 (e.g., 0, 1, 2, or 3) nucleotides from the nucleotide sequence of the antisense strands of any one of dsRNA agents 1-1590 (e.g., one of dsRNA agents 1-16 or 770-785) set forth in any one of Tables 2-5 (e.g., Table 3 or 5); and wherein the sense strand comprises at least 15 (e.g., 16, 17, 18, 19, 20, 21, 22, or 23) contiguous nucleotides of and differing by no more than 3 (e.g., 0, 1, 2, or 3) nucleotides from the nucleotide sequence of the sense strand of the corresponding dsRNA agent set forth in any one of Tables 2-5 (e.g., Table 3 or 5).

It should be understood that each of the following embodiments is applicable to each and any of the foregoing aspects as if recited directed after each aspect.

In some embodiments, the sense strand comprises at least 15 (e.g., 16, 17, 18, 19, 20, 21) contiguous nucleotides of and differing by no more than 3 (e.g., 0, 1, 2, or 3) nucleotides from any sense strand set forth in any one of Tables 2-5 (e.g., Table 3 or 5) or set forth in any one of SEQ ID NOS: 1577-3166 (e.g., any one of SEQ ID NOS: 1577-1592 or 2346-2361).

In some embodiments, the sense strand comprises at least 15 (e.g., 16, 17, 18, 19, 20, 21) contiguous nucleotides of and differing by no more than 3 (e.g., 0, 1, 2, or 3) nucleotides from the corresponding sense strand set forth in any one of Tables 2-5 (e.g., Table 3 or 5) or the corresponding sense strand set forth in one of SEQ ID NOS: 1577-3166.

In some embodiments, the sense strand comprises at least one modified nucleotide and/or the antisense strand comprises at least one modified nucleotide.

In some embodiments, the sense strand comprises at least one modified nucleotide and the antisense strand comprises at least one modified nucleotide.

In some embodiments, at least 25%, 30%, 35%, 40%, 45%, 50%, 55%, 60%, 65%, 70%, 75%, 80%, 85%, 90%, 95%, or 100% of the nucleotides of the sense strand and/or antisense strand are modified.

In some embodiments, substantially all (or all) of the nucleotides in the sense strand and/or antisense strand are modified. In some embodiments, at least one of the modified nucleotides comprises a modified sugar (e.g., ribose moiety). In some embodiments, at least one of the modified nucleotides comprises a modified nucleobase. In some embodiments, at least one of the modified nucleotides comprises a modified sugar (e.g., ribose moiety) and a modified nucleobase. In some embodiments, the sense strand comprises at least one modified internucleoside linkage and/or the antisense strand comprises at least one modified internucleoside linkage. In some embodiments, the sense strand comprises at least one modified internucleoside linkage and the antisense strand comprises at least one modified internucleoside linkage.

In some embodiments, each of the antisense strand and the sense strand are not more than 30, 29, 28, 27, 26, 25, 24, 23, 22, 21, 20, 19, 18, 17, 16, or 15 nucleotides in length. In some embodiments, the antisense strand comprises from about 15-30, 16-30, 17-30, 18-30, 19-30 20-30, 21-30, 22-30, 23-30, 24-30, 25-30, 36-30, 27-30, 28-30-, 29-30, 19-20, 19-21, 19-22, 19-23, 19-24, or 19-25 nucleotides; and/or the sense strand comprises from about 15-30, 16-30, 17-30, 18-30, 19-30 20-30, 21-30, 22-30, 23-30, 24-30, 25-30, 36-30, 27-30, 28-30-, 29-30, 19-20, 19-21, 19-22, 19-23, 19-24, or 19-25 nucleotides. In some embodiments, antisense strand comprises from about 19-23 nucleotides; and/or the sense strand comprises from about 19-23 nucleotides. In some embodiments, antisense strand consists of 23 nucleotides; and the sense strand consists of 21 nucleotides. In some embodiments, antisense strand comprises or consists of about 23 nucleotides; and/or the sense strand comprises or consists of about 21 nucleotides.

In some embodiments, the sense strand and/or the antisense strand comprises a 3′ and/or 5′ overhang of 1, 2, or 3 nucleotides. In some embodiments, the antisense strand comprises a 3′ overhang of 1, 2, or 3 nucleotides (e.g., 2 nucleotides).

In some embodiments, the double stranded region is from about 19-30, 19-29, 19-28, 19-27, 19-26, 19-25, 19-24, 19-23, 19-22, 19-20, 19-21, 23-30, 23-29, 23-28, 23-27, 23-26, 23-25, 23-24, 21-30, 21-29, 21-28, 21-27, 21-26, 21-25, 21-24, 21-23, or 21-22 nucleotide pairs in length. In some embodiments, the double stranded region is from about 19-23 or 19-21 nucleotide pairs in length. In some embodiments, the double stranded region is about 21 nucleotide pairs in length.

In some embodiments, the sense strand and the antisense strand are part of a single nucleic acid molecule (e.g., wherein a hairpin loop is between the sense strand and the antisense strand of the single nucleic acid molecule. In some embodiments, the sense strand and the antisense strand are separate nucleic acid molecules (i.e., connected only through the double stranded region).

In one aspect, provided herein are antisense oligonucleotide agents for inhibiting expression of BCL11) (e.g., hBCL11A), wherein the antisense oligonucleotide comprises at least 15 (e.g., 16, 17, 18, 19, 20) contiguous nucleotides of and differing by no more than 3 (e.g., 0, 1, 2, or 3) nucleotides from the nucleotide sequence of any one of the antisense oligonucleotides set forth in Table 7 or 9 (e.g., Table 9) or set forth in any one of SEQ ID NOS: 3167-3280.

In some embodiments, the antisense oligonucleotide comprises the nucleotide sequence of any one of the antisense oligonucleotides set forth in Table 7 or 9 (e.g., Table 9) or set forth in any one of SEQ ID NOS: 3167-3280. In some embodiments, the antisense oligonucleotide consists of the nucleotide sequence of any one of the antisense oligonucleotides set forth in Table 7 or 9 (e.g., Table 9) or set forth in any one of SEQ ID NOS: 3167-3280.

In some embodiments, the antisense oligonucleotide comprises at least one modified nucleotide. In some embodiments, at least 25%, 30%, 35%, 40%, 45%, 50%, 55%, 60%, 65%, 70%, 75%, 80%, 85%, 90%, 95%, or 100% of the nucleotides of the antisense oligonucleotide are modified. In some embodiments, substantially all (or all) of the nucleotides in the antisense oligonucleotide are modified. In some embodiments, at least one of the modified nucleotides comprises a modified sugar (e.g., ribose moiety, deoxyribose moiety). In some embodiments, the antisense oligonucleotide comprises one or more 2′-O-methoxyethyl moiety (e.g., ribose moiety, deoxyribose moiety). In some embodiments, the antisense oligonucleotide comprises one or more locked nucleic acid (LNA). In some embodiments, at least one of the modified nucleotides comprises a modified nucleobase. In some embodiments, the antisense oligonucleotide comprises at least one modified internucleoside linkage. In some embodiments, the antisense oligonucleotide comprises one or more phosphorothioate linkage.

In some embodiments, each of the antisense oligonucleotide is not more than 30, 29, 28, 27, 26, 25, 24, 23, 22, 21, 20, 19, 18, 17, 16, or 15 nucleotides in length. In some embodiments, the antisense oligonucleotide comprises from about 10-30 (e.g., 15-29, 15-28, 15-27, 15-26, 15-25, 15-24, 15-23, 15-22, 15-21, 15-20, 15-19, 15-18, 15-17, 15-16, 16-29, 16-28, 16-27, 16-26, 16-25, 16-24, 16-23, 16-22, 16-21, 16-20, 16-19, 16-18, 16-17, 17-29, 17-28, 17-27, 17-26, 17-25, 17-24, 17-23, 17-22, 17-21, 17-20, 17-19, 17-18, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, or 19-20 nucleotides. In some embodiments, the antisense oligonucleotide comprises from about 15-25, 15-14, 15-23, 15-22, 15-21, 15-20, 15-19, 15-18, 15-17, 15-16, 16-25, 16-14, 16-23, 16-22, 16-21, 16-20, 16-19, 16-18, 16-17, 17-25, 17-14, 17-23, 17-22, 17-21, 17-20, 17-19, 17-18, 18-25, 18-14, 18-23, 18-22, 18-21, 18-20, 18-19, 19-25, 19-14, 19-23, 19-22, 19-21, or 19-20 nucleotides. In some embodiments, the antisense oligonucleotide comprises from about 15-20 nucleotides. In some embodiments, the antisense oligonucleotide comprises about 15, 16, 17, 18, 19, 20, 21, 22, 23, 24, 25, 26, 27, 28, 29, or 30 nucleotides. In some embodiments, the antisense oligonucleotide comprises about 15, 16, 17, 18, 19, or 20 nucleotides. In some embodiments, the antisense oligonucleotide consists of about 15, 16, 17, 18, 19, 20, 21, 22, 23, 24, 25, 26, 27, 28, 29, or 30 nucleotides. In some embodiments, the antisense oligonucleotide consist of about 15, 16, 17, 18, 19, or 20 nucleotides.

In one aspect, provided herein are conjugates comprising (i) a dsRNA agent described herein or an antisense oligonucleotide described herein and (ii) a heterologous moiety.

In some embodiments, the heterologous moiety is a peptide, protein, carbohydrate, lipid, polymer, or small molecule.

In some embodiments, the heterologous moiety is a protein.

In some embodiments, the protein specifically binds to a moiety expressed by a hematopoietic cell (e.g., on the surface of a hematopoietic cell). In some embodiments, the protein specifically binds to a moiety expressed by an erythroid precursor cell (e.g., on the surface of an erythroid precursor cell). In some embodiments, the protein specifically binds the transferrin receptor (TFR) (e.g., human TFR (hTFR) (e.g., hTFR1)).

In some embodiments, the protein is an antibody. In some embodiments, the antibody comprises or consists of a full-length antibody, a Fab, a Fab′, a F(ab′)2, a Fab-Fc, a scFv, a scFv-Fc, a (scFv)₂-Fc, an Fv, a single domain antibody (sdAb) (e.g., a VHH), a sdAb-Fc (e.g., a VHH-Fc), a (sdAb)₂ (e.g., a (VHH)₂), or a (sdAb)₂-Fc (e.g., a (VHH)₂-Fc). In some embodiments, the antibody comprises or consists of a full-length antibody, a Fab-Fc, a scFv-Fc, a (scFv)₂-Fc, a sdAb-Fc (e.g., a VHH-Fc), or a (sdAb)₂-Fc (e.g., a (VHH)₂-Fc).

In some embodiments, the antibody is an IgG (e.g., a human IgG (hIgG)) antibody. In some embodiments, the antibody is a hIgG1, hIgG2, hIgG3, or hIgG4 antibody (e.g., a hIgG1 or hIgG4 antibody). In some embodiments, the antibody comprises an immunoglobulin (Ig) (e.g., a human Ig (hIg)) Fc region. In some embodiments, the Ig (e.g., hIg) Fc region comprises at least a portion of a hinge region, a CH2 region, and a CH3 region. In some embodiments, the Ig (e.g., hIg) Fc region comprises a hinge region, a CH2 region, and a CH3 region. In some embodiments, the Ig is a hIg. In some embodiments, the hIg is a human IgG (hIgG). In some embodiments, the hIgG is hIgG1 or hIgG4. In some embodiments, the Ig (e.g., hIg) Fc region comprises one or more amino acid substitutions relative to a reference Ig (e.g., hIg) Fc region that reduces or abolishes one or more of the following effector functions relative to the reference hIg Fc region: antibody dependent cell mediated cytotoxicity (ADCC), complement dependent cytotoxicity (CDC), and/or affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In some embodiments, the antibody does not (or does not substantially) block binding of TF (e.g., hTF) to the TFR (e.g., hTFR1).

In some embodiments, the protein is a TFR ligand (or a functional fragment or functional variant thereof).

In some embodiments, the TFR ligand comprises transferrin (TF) (e.g., human transferrin (hTF)) (or a functional fragment or functional variant thereof).

In some embodiments, the heterologous moiety is a targeting moiety.

In some embodiments, the targeting moiety specifically binds to a moiety expressed by a hematopoietic cell (e.g., on the surface of a hematopoietic cell). In some embodiments, the targeting moiety specifically binds to a moiety expressed by an erythroid precursor cell (e.g., on the surface of an erythroid precursor cell). In some embodiments, the targeting moiety specifically binds TFR (e.g., hTFR (e.g., hTFR1)).

In some embodiments, the targeting moiety is a peptide, protein, carbohydrate, lipid, polymer, or small molecule.

In some embodiments, the targeting moiety is a protein.

In some embodiments, the targeting moiety is an antibody. In some embodiments, the antibody comprises or consists of a full-length antibody, a Fab, a Fab′, a F(ab′)2, a Fab-Fc, a scFv, a scFv-Fc, a (scFv)₂-Fc, an Fv, a single domain antibody (sdAb) (e.g., a VHH), a sdAb-Fc (e.g., a VHH-Fc), a (sdAb)₂ (e.g., a (VHH)₂), or a (sdAb)₂-Fc (e.g., a (VHH)₂-Fc). In some embodiments, the antibody comprises or consists of a full-length antibody, a Fab-Fc, a scFv-Fc, a (scFv)₂-Fc, a sdAb-Fc (e.g., a VHH-Fc), or a (sdAb)₂-Fc (e.g., a (VHH)₂-Fc).

In some embodiments, the antibody is an IgG (e.g., a human IgG (hIgG)) antibody. In some embodiments, the antibody is a hIgG1, hIgG2, hIgG3, or hIgG4 antibody (e.g., a hIgG1 or hIgG4 antibody). In some embodiments, the antibody comprises an immunoglobulin (Ig) (e.g., a human Ig (hIg)) Fc region. In some embodiments, the Ig (e.g., hIg) Fc region comprises at least a portion of a hinge region, a CH2 region, and a CH3 region. In some embodiments, the Ig (e.g., hIg) Fc region comprises a hinge region, a CH2 region, and a CH3 region. In some embodiments, the Ig is a hIg. In some embodiments, the hIg is a human IgG (hIgG). In some embodiments, the hIgG is hIgG1 or hIgG4. In some embodiments, the Ig (e.g., hIg) Fc region comprises one or more amino acid substitutions relative to a reference Ig (e.g., hIg) Fc region that reduces or abolishes one or more of the following effector functions relative to the reference hIg Fc region: antibody dependent cell mediated cytotoxicity (ADCC), complement dependent cytotoxicity (CDC), and/or affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In some embodiments, the antibody does not (or does not substantially) block binding of TF (e.g., hTF) to the TFR (e.g., hTFR1).

In some embodiments, the targeting moiety is a TFR ligand (or a functional fragment or functional variant thereof).

In some embodiments, the TFR ligand comprises transferrin (TF) (e.g., human transferrin (hTF)) (or a functional fragment or functional variant thereof).

In some embodiments, the conjugate exhibits one or more of the following properties: (a) upon binding to TFR (e.g., hTFR (e.g., TFR1)) expressed on the surface of a hematopoietic cell, the conjugate does not induce death of the target cell; (b) upon binding to TFR (e.g., hTFR (e.g., TFR1)) expressed on the surface of a hematopoietic cell, the hematopoietic cell remains viable; (c) upon internalization into a hematopoietic cell, the conjugate does not induce death of the hematopoietic cell; and/or (d) upon binding to TFR (e.g., hTFR (e.g., TFR1)) expressed on the surface of a hematopoietic cell, the conjugate does not induce degradation of TFR (e.g., hTFR (e.g., TFR1)).

In some embodiments, the conjugate exhibits one or more of the following properties: (a) upon binding to TFR (e.g., hTFR (e.g., TFR1)) expressed on the surface of a hematopoietic cell, the conjugate is internalized into the hematopoietic cell; (b) upon binding to TFR (e.g., hTFR (e.g., TFR1)) expressed on the surface of a hematopoietic cell, the conjugate does not induce death of the target cell; (c) upon binding to TFR (e.g., hTFR (e.g., TFR1)) expressed on the surface of a hematopoietic cell, the hematopoietic cell remains viable; (d) upon internalization into a hematopoietic cell, the conjugate does not induce death of the hematopoietic cell; and/or (e) upon binding to TFR (e.g., hTFR (e.g., TFR1)) expressed on the surface of a hematopoietic cell, the conjugate does not induce degradation of TFR (e.g., hTFR (e.g., TFR1)).

In some embodiments, the protein (e.g., an antibody) that specifically binds to the TFR (e.g., hTFR (e.g., hTFR1)) exhibits one or more of the following properties: (a) upon binding to TFR (e.g., hTFR (e.g., TFR1)) expressed on the surface of a hematopoietic cell, the protein (e.g., an antibody) that specifically binds to the TFR (e.g., hTFR (e.g., hTFR1)) or conjugate is internalized into the hematopoietic cell; (b) upon binding to TFR (e.g., hTFR (e.g., TFR1)) expressed on the surface of a hematopoietic cell, the protein (e.g., an antibody) that specifically binds to the TFR (e.g., hTFR (e.g., hTFR1)) or conjugate does not induce death of the target cell; (c) upon binding to TFR (e.g., hTFR (e.g., TFR1)) expressed on the surface of a hematopoietic cell, the hematopoietic cell remains viable; (d) upon internalization into a hematopoietic cell, the protein (e.g., an antibody) that specifically binds to the TFR (e.g., hTFR (e.g., hTFR1)) or conjugate does not induce death of the hematopoietic cell; and/or (e) upon binding to TFR (e.g., hTFR (e.g., TFR1)) expressed on the surface of a hematopoietic cell, the protein (e.g., an antibody) that specifically binds to the TFR (e.g., hTFR (e.g., hTFR1)) or conjugate does not induce degradation of TFR (e.g., hTFR (e.g., TFR1)).

In some embodiments, the heterologous moiety is attached to the dsRNA agent or the antisense oligonucleotide via a linker. In some embodiments, the linker is cleavable.

In some embodiments, the heterologous moiety attached to the 3′ end of the sense and/or antisense strand and/or the 5′ end of the sense and/or antisense strand, and/or at an internal site of the sense and/or antisense strand. In some embodiments, the heterologous moiety attached to the 3′ end of the antisense oligonucleotide, the 5′ end of the antisense oligonucleotide, and/or at an internal site of the antisense oligonucleotide.

In one aspect, provided herein are vectors (e.g., a viral vector, a non-viral vector) encoding (i) an antisense strand, a sense strand, or both an antisense and sense strand described herein or (ii) or an antisense oligonucleotide described herein.

In one aspect, provided herein are carriers comprising a dsRNA agent described herein, an antisense oligonucleotide described herein, a conjugate described herein, or a vector described herein.

In some embodiments, the carrier comprises a nanoparticle, a polymer, a lipid-based delivery system, as dendrimer, a cationic delivery system, or a hydrogel. In some embodiments, the lipid-based delivery system is a lipid nanoparticle (LNP), liposome, lipoplex, nanoliposome, an exosome, or a micelle.

In one aspect, provided herein are cells comprising a dsRNA agent described herein, an antisense oligonucleotide described herein, a conjugate described herein, a vector described herein, or a carrier described herein.

In one aspect, provided herein are pharmaceutical compositions comprising a dsRNA agent described herein, an antisense oligonucleotide described herein, a conjugate described herein, a vector described herein, a carrier described herein, or a cell described herein, and a pharmaceutically acceptable excipient.

In one aspect, provided herein are kits comprising a dsRNA agent described herein, an antisense oligonucleotide described herein, a conjugate described herein, a vector described herein, a carrier described herein, a cell described herein, or a pharmaceutical composition described herein.

In one aspect, provided herein are methods of delivering a dsRNA, antisense oligonucleotide, conjugate, vector, carrier, or pharmaceutical composition to a cell, the method comprising introducing into a cell a dsRNA agent described herein, an antisense oligonucleotide described herein, a conjugate described herein, a vector described herein, a carrier described herein, or a pharmaceutical composition described herein, to thereby deliver the dsRNA, antisense oligonucleotide, conjugate, vector, carrier, or pharmaceutical composition into the cell.

In some embodiments, the cell is in vitro, ex vivo, or in vivo. In some embodiments, the cell is a subject (e.g., a human subject).

In one aspect, provided herein are methods of delivering a dsRNA, antisense oligonucleotide, conjugate, vector, carrier, cell, or pharmaceutical composition to a subject, the method comprising administering to the subject a dsRNA agent described herein, an antisense oligonucleotide described herein, a conjugate described herein, a vector described herein, a carrier described herein, a cell described herein, or a pharmaceutical composition described herein, to thereby deliver the dsRNA, antisense oligonucleotide, conjugate, vector, carrier, cell, or pharmaceutical composition to the subject.

In one aspect, provided herein are methods of reducing or inhibiting expression of BCL11A (e.g., hBCL11A) in a cell, the method comprising delivering into the cell a dsRNA agent described herein, an antisense oligonucleotide described herein, a conjugate described herein, a vector described herein, a carrier described herein, a cell described herein, or a pharmaceutical composition described herein, to thereby reduce or inhibit expression of BCL11A (e.g., hBCL11A) in the cell.

In one aspect, provided herein are methods of reducing or inhibiting expression of BCL11A (e.g., hBCL11A) in a cell in a subject, the method comprising administering to the subject a dsRNA agent described herein, an antisense oligonucleotide described herein, a conjugate described herein, a vector described herein, a carrier described herein, a cell described herein, or a pharmaceutical composition described herein, to thereby reduce or inhibit expression of BCL11A (e.g., hBCL11A) in the cell in the subject.

In one aspect, provided herein are methods of treating, ameliorating, or preventing a BCL11A (e.g., hBCL11A) associated disease in a subject, the method comprising administering to the subject a dsRNA agent described herein, an antisense oligonucleotide described herein, a conjugate described herein, a vector described herein, a carrier described herein, a cell described herein, or a pharmaceutical composition described herein, to thereby treat, ameliorate, or prevent the BCL11A (e.g., hBCL11A) associated disease in the subject.

In some embodiments, the BCL11A associated disease is an inherited blood disorder.

In some embodiments, the inherited blood disorder is a hemoglobinopathy. In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, or methemoglobinemia.

In some embodiments, the inherited blood disorder is an inherited bone marrow failure syndrome. In some embodiments, the inherited bone marrow failure syndrome is amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, thrombocytopenia absent radii (TAR), a SAMD9/SAMD9L disorder, or a MECOM-associated syndrome.

In one aspect, provided herein are methods of treating, ameliorating, or preventing an inherited blood disorder in a subject, the method comprising administering to the subject a dsRNA agent described herein, an antisense oligonucleotide described herein, a conjugate described herein, a vector described herein, a carrier described herein, a cell described herein, or a pharmaceutical composition described herein, to thereby treat, ameliorate, or prevent the inherited blood disorder in the subject.

In some embodiments, the inherited blood disorder is a hemoglobinopathy. In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, or methemoglobinemia.

In one aspect, provided herein are methods of treating, ameliorating, or preventing a hemoglobinopathy in a subject, the method comprising administering to the subject a dsRNA agent described herein, an antisense oligonucleotide described herein, a conjugate described herein, a vector described herein, a carrier described herein, a cell described herein, or a pharmaceutical composition described herein, to thereby treat, ameliorate, or prevent the hemoglobinopathy in the subject.

In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, or methemoglobinemia.

In one aspect, provided herein are methods of treating, ameliorating, or preventing an inherited bone marrow failure syndrome in a subject, the method comprising administering to the subject a dsRNA agent described herein, an antisense oligonucleotide described herein, a conjugate described herein, a vector described herein, a carrier described herein, a cell described herein, or a pharmaceutical composition described herein, to thereby treat, ameliorate, or prevent the inherited bone marrow failure syndrome in the subject.

In some embodiments, the inherited bone marrow failure syndrome is amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, thrombocytopenia absent radii (TAR), a SAMD9/SAMD9L disorder, or a MECOM-associated syndrome.

In one aspect, provided herein are methods of inducing expression of fetal hemoglobin in a subject, the method comprising administering to the subject a dsRNA agent described herein, an antisense oligonucleotide described herein, a conjugate described herein, a vector described herein, a carrier described herein, a cell described herein, or a pharmaceutical composition described herein, to thereby induce expression of fetal hemoglobin the subject.

In one aspect, provided herein are methods of increasing the level of fetal hemoglobin in a subject, the method comprising administering to the subject a dsRNA agent described herein, an antisense oligonucleotide described herein, a conjugate described herein, a vector described herein, a carrier described herein, a cell described herein, or a pharmaceutical composition described herein, to thereby increase the level of fetal hemoglobin the subject.

In one aspect, provided herein are methods of increasing the ratio of fetal hemoglobin to adult hemoglobin in a subject, the method comprising administering to the subject a dsRNA agent described herein, an antisense oligonucleotide described herein, a conjugate described herein, a vector described herein, a carrier described herein, a cell described herein, or a pharmaceutical composition described herein, to thereby increase the ratio of fetal hemoglobin to adult hemoglobin in the subject.

In some embodiments, the subject is a human.

### 4. DETAILED DESCRIPTION

The inventors have further discovered, inter alia, RNA agents (e.g., siRNA agents; antisense oligonucleotides) that inhibit expression of BCL11A (e.g., hBCL11A). As such, the RNA agents described herein are useful for the treatment of BCL11A mediated diseases. As such, the current disclosure provides RNA agents (e.g., dsRNAi agents comprising a sense strand and an antisense strand, siRNA agents; ssRNA agents, antisense oligonucleotides, etc.) capable of inhibiting BCL11A expression (e.g., in a cell, in a cell in a subject); and their use in, inter alia, pharmaceutical compositions, and methods of treating diseases (e.g., BCL11A mediated diseases).

TABLE OF CONTENTS

| | |
|---|---|
| 4.2 | RNAi Agents |
| 4.2.1 | Antisense Strand |
| 4.2.1.1 | Targeting Region |
| 4.2.1.2 | Overall Length |
| 4.2.1.3 | Exemplary Antisense Strands |
| 4.2.2 | Sense Strand |
| 4.2.2.1 | Antisense Strand Complementarity |
| 4.2.2.2 | Overall Length |
| 4.2.2.3 | Exemplary Sense Strands |
| 4.2.3 | dsRNA Agents |
| 4.2.3.1 | Single & Multiple Nucleic Acid Molecules |
| 4.2.3.2 | Length of Double Stranded Region |
| 4.2.3.3 | Nucleotide Overhangs & Blunt Ends |
| 4.2.3.4 | Exemplary Structural Combinations of Sense & Antisense Strands |
| 4.2.3.5 | Exemplary Antisense Strands & Sense Strands |
| 4.2.3.6 | Exemplary dsRNA Agents |
| 4.3 | Antisense Oligonucleotides |
| 4.3.1 | Targeting Region |
| 4.3.2 | Overall Length |
| 4.3.3 | Exemplary Antisense Oligonucleotides |
| 4.3.4 | Antisense Oligonucleotides |
| 4.4 | Modified Nucleic Acid Molecule Agents (e.g., dsRNA agents, ASOs) |
| 4.4.1 | Nature of Nucleotide Modifications |
| 4.4.1.1 | Modified Nucleosides |
| 4.4.1.1(i) | Sugar Modifications |
| 4.4.1.1(i)(a) | Non-Bicyclic Sugar Modifications |
| 4.4.1.1(i)(b) | Bicyclic Sugar Modifications |
| 4.4.1.1(ii) | Nucleobase Modifications |
| 4.4.1.2 | Internucleoside Linkage Modifications |
| 4.4.1.2(i) | Modified Phosphorous Containing Internucleoside Linkages |
| 4.4.1.2(ii) | Modified Non-Phosphorous Containing Internucleoside Linkages |
| 4.4.1.3 | Additional Exemplary Nucleotide Modifications |
| 4.4.2 | Extent of Modified Nucleotides |
| 4.5 | Conjugates |
| 4.5.1 | Heterologous Moieties |
| 4.5.1.1 | Targeting Moieties |
| 4.5.1.1(i) | Hematopoietic Cell Targeting Moieties |
| 4.5.4.4(i)(a) | TFR Targeting Moieties |
| 4.5.4.4(i)(b) | Ig Constant Regions |
| 4.5.2 | Linkers |
| 4.5.2.1 | Cleavable Linkers |
| 4.5.3 | Orientation |
| 4.6 | Activity of Nucleic Acis Molecule Agents & Conjugates Thereof |
| 4.7 | Methods of Making Nucleic Acid Molecule Agents & Conjugates Thereof |
| 4.8 | Methods of Making Proteins (e.g., Targeting Moieties) |
| 4.9 | Vectors |
| 4.10 | Carriers |
| 4.11 | Host Cells |
| 4.12 | Pharmaceutical Compositions |
| 4.13 | Methods of Use |
| 4.13.1 | Methods of Delivery |
| 4.13.2 | Methods of Reducing or Inhibiting BCL11A Expression |
| 4.13.3 | Methods of Inducing Expression of Fetal Hemoglobin |
| 4.13.4 | Methods of Treating, Ameliorating, or Preventing a BCL11A Associated |
| | Disease |
| 4.13.5 | Methods of Treating, Ameliorating, or Preventing an Inherited Blood |
| | Disorder |
| 4.13.6 | Methods of Treating, Ameliorating, or Preventing a Hemoglobinopathy |
| 4.13.7 | Methods of Treating, Ameliorating, or Preventing an Inherited Bone |
| | Marrow Failure Syndrome |
| 4.14 | Kits |
| 4.1 | Definitions |

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this application, the use of the singular includes the plural unless specifically stated otherwise. For example, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” or “comprising essentially of” should be assumed to be within an acceptable error range for that particular value or composition.

As used herein, the term “administering” refers to the physical introduction of an agent, e.g., a therapeutic agent (or a precursor of the therapeutic agent that is metabolized or altered within the body of the subject to produce the therapeutic agent in vivo) to a subject, using any of the various methods and delivery systems known to those skilled in the art. Administering can also be performed, for example, once, a plurality of times, and/or over one or more extended periods. Administering includes both self-administration by the subject and administration to the subject by a third party.

As used herein, the terms “agent” and “moiety” are used interchangeable herein and are used generically to describe any macro or micro molecule. Exemplary moieties include, but are not limited polypeptides, proteins, peptides, polynucleotides (e.g., DNA, RNA), small molecules, carbohydrates, lipids, synthetic polymers (e.g., polymers of PEG) (or any combinations thereof). In some embodiments, the term “moiety” is used to describe any macro or micro molecule that can be operably connected to a dsRNA agent described herein. For example, proteins (e.g., antibodies (e.g., anti-TFR antibodies)), targeting moieties, etc., that can be operable connected to a dsRNA agent described herein.

As used herein, the term “affinity” refers to the strength of the binding of one protein (e.g., an Antibody) to another protein (e.g., an Antigen). The affinity of a protein is measured by the dissociation constant Kd, defined as [Antibody]×[Antigen]/[Antibody-Antigen] where [Antibody-Antigen] is the molar concentration of the Antibody-Antigen complex, [Antibody] is the molar concentration of the unbound Antibody and [Ligand] is the molar concentration of the unbound Antigen. The affinity constant Ka is defined by 1/Kd. Standard methods of measuring affinity are known to the person of ordinary skill in the art. Exemplary methods of measuring affinity include, surface plasmon resonance (SPR) (e.g., BIAcore®-based assay), a common method known in the art (see, e.g., Wilson, Science 295:2103, 2002; Wolff et al., Cancer Res. 55:2560, 1993; and U.S. Pat. Nos. 5,283,173, 5,468,614, the full contents of each of which are incorporated by reference herein for all purposes).

As used herein, the term “antibody” or “antibodies” is used in the broadest sense and encompasses various immunoglobulin (Ig) (e.g., human Ig (hIg), murine Ig (mIg)) structures, including, but not limited to monoclonal antibodies, polyclonal antibodies, multispecific (e.g., bispecific, trispecific) antibodies, and antibody fragments so long as they exhibit the desired antigen-binding activity (i.e., antigen binding fragments or variants). The term antibody thus includes, for example, full-length antibodies; antigen-binding fragments of full-length antibodies; molecules comprising antibody CDRs, VH regions, and/or VL regions; and antibody-like scaffolds (e.g., fibronectins). Examples of antibodies include, without limitation, monoclonal antibodies, polyclonal antibodies, monospecific antibodies, multispecific antibodies, human antibodies, humanized antibodies, chimeric antibodies, camelized antibodies, intrabodies, a variable domain of a new antigen receptor beta-lactamase (VNAR fragments), affybodies, diabodies, tribodies, heteroconjugate antibodies, antibody-drug conjugates, single domain antibodies (e.g., VHH, (VHH)₂), single chain antibodies, single-chain Fvs (scFv; (scFv)₂), Fab fragments (e.g., Fab, single chain Fab (scFab), F(ab′)₂ fragments, disulfide-linked Fvs (sdFv), Fc fusions (e.g., Fab-Fc, scFv-Fc, VHH-Fc, (scFv)₂-Fc, (VHH)₂-Fc), and antigen-binding fragments of any of the above, and conjugates or fusion proteins comprising any of the above. Antibodies can be of Ig isotype (e.g., IgG, IgE, IgM, IgD, or IgA), any class (e.g., IgG₁, IgG₂, IgG₃, IgG₄, IgA₁ or IgA₂), or any subclass (e.g., IgG₂ₐ or IgG_{2b}) of Ig). In certain embodiments, antibodies described herein are IgG antibodies, or a class (e.g., human IgG₁ or IgG₄) or subclass thereof. In certain embodiments, antibodies described herein are mIgG antibodies, or a class (e.g., mIgG1 or mIgG2a) or subclass thereof. In some embodiments, the antibody is a human, humanized, or chimeric IgG₁ or IgG₄ monoclonal antibody. In some embodiments, the term antibodies refers to a monoclonal or polyclonal antibody population. Antibodies described herein can be produced by any standard methods known in the art, e.g., recombinant production in host cells, see, e.g., § 4.8; or synthetic production.

As used herein, the term “antibody-like scaffold” refers to non-Ig based antigen binding domain. Various antibody-like scaffolds are known in the art. For example, 10th type III domain of fibronectin (e.g., AdNectins®) and designed ankyrin repeat proteins (e.g., DARPins®) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008), the full contents of each of which is incorporated by reference herein for all purposes. Exemplary antibody-like scaffolds include, but are not limited to, lipocalins (see, e.g., U.S. Pat. No. 7,250,297) (e.g., Anticalin®), protein A-derived molecules such as z-domains of protein a (see, e.g., U.S. Pat. No. 5,831,012) (e.g., Affibody®), A domains of membrane receptors stabilized by disulfide bonds and Ca2+ (see, e.g., U.S. Pat. No. 7,803,907) (e.g., Avimer/Maxibody®), a serum transferrin (see, e.g., US2004023334) (e.g., Transbody®); a designed ankyrin repeat protein (see, e.g., U.S. Pat. No. 7,417,130) (e.g., DARPin®), a fibronectin (see, e.g., U.S. Pat. No. 6,818,418) (e.g., AdNectin®), a C-type lectin domain (see, e.g., US2004132094) (e.g., Tetranectin®); a human gamma-crystallin or ubiquitin (see, e.g., U.S. Pat. No. 7,838,629) (e.g., Affilin®); a kunitz type domain of human protease inhibitors (see, e.g., US2004209243), C-Type Lectins (see, e.g., US2004132094) (e.g., Tetranectins®), cysteine knots or knottins (see, e.g., U.S. Pat. No. 7,186,524) (e.g., Microbodies®), nucleic acid aptamers (see, e.g., U.S. Pat. No. 5,475,096), thioredoxin A scaffold (see, e.g., U.S. Pat. No. 6,004,746) (peptide aptamers), and 10th type III domain of fibronectin (see, e.g., U.S. Pat. No. 6,818,418) (e.g., AdNectins®), and cystine-dense peptides (see, e.g., WO2023023031). Additional exemplary antibody-like scaffolds are known in the art and for example described in Storz U. Intellectual property protection: strategies for antibody inventions. MAbs. 2011; 3(3):310-317. doi:10.4161/mabs.3.3.15530. The entire contents of each of the foregoing references is incorporated herein by reference for all purposes. Antibody like scaffolds include e.g., naturally occurring antigen binders, variant (e.g., functional variants) of naturally occurring antigen binders, fragments (e.g., functional fragments) of naturally occurring antigen binders, and synthetic antigen binders (i.e., not naturally occurring antigen binders).

As used herein, the term “antibody dependent cell mediated cytotoxicity” or “ADCC” refers to an immune mechanism leading to the lysis of antibody (or an Fc region containing protein) (e.g., an Ig Fc containing fusion protein described herein)-coated target cells by immune effector cells (e.g., NK cells). As used herein, the term “reduced ADCC” and the like refers to either a reduction in the number of target cells that are lysed in a given time, at a given concentration of antibody (or an Ig Fc region containing protein) (e.g., an Fc region containing fusion protein described herein) in the medium surrounding the target cells, by the mechanism of ADCC defined above, and/or an increase in the concentration of antibody (or an Fc region containing protein) (e.g., an Fc containing fusion protein described herein) in the medium surrounding the target cells, required to achieve the lysis of a given number of target cells in a given time, by the mechanism of ADCC defined above. The reduction in ADCC is relative to the ADCC mediated by the same antibody (or an Fc region containing protein) (e.g., an Fc containing fusion protein described herein) produced by the same type of host cells, using the same standard production, purification, formulation and storage methods (which are known to those skilled in the art), but that has not been engineered (e.g., does not comprise one or more amino acid variation, e.g., amino acid substitution, that mediates a decrease in ADCC). For example the reduction in ADCC mediated by an antibody (or an Fc region containing protein) (e.g., an Fc containing fusion protein described herein) comprising in its Fc region an amino acid substitution that reduces ADCC, is relative to the ADCC mediated by the same antibody (or an Fc region containing protein) (e.g., an Fc containing fusion protein described herein) without said amino acid substitution in the Fc region.

As used herein, the term “antisense polynucleotide” and the like refers to a polynucleotide capable of undergoing hybridization to a target nucleic acid molecule through hydrogen bonding. Examples of antisense polynucleotides include single-stranded and double-stranded polynucleotides, such as, antisense oligonucleotides (ASOs), siRNAs, shRNAs, ssRNAs. In some embodiments, the antisense polynucleotide is an RNA molecule. In some embodiments, the antisense polynucleotide is a DNA molecule. In some embodiments, the antisense polynucleotide is a DNA/RNA hybrid molecule.

“Antisense oligonucleotide” refers to an antisense polynucleotide that is a single-stranded oligonucleotide having a nucleobase sequence that permits hybridization to a corresponding segment of a target nucleic acid. In some embodiments, the antisense oligonucleotide is an RNA molecule. In some embodiments, the antisense oligonucleotide is a DNA molecule. In some embodiments, the antisense oligonucleotide is a DNA/RNA hybrid molecule.

As used herein, the term “antisense strand” refers to an antisense polynucleotide that is an RNA molecule that is part of an RNAi agent (e.g., described herein) or part of a dsRNA agent (e.g., described herein) (e.g., an siRNA agent described herein) that comprises a region of complementarity comprising a nucleotide sequence that is at least partially (e.g., substantially, fully) complementary to a target nucleic acid sequence (e.g., a target mRNA (e.g., a BCL11A mRNA), a portion of a target mRNA (e.g., a BCL11A mRNA)), such that it is capable of undergoing hybridization to a target nucleic acid molecule through hydrogen bonding.

As used herein, the term “bicyclic sugar” refers to a modified sugar (e.g., ribose) moiety comprising two rings, wherein the second ring is formed via a bridge connecting two of the atoms in the first ring thereby forming a bicyclic structure. In some embodiments, the first ring of the bicyclic sugar moiety is a furanosyl moiety. In some embodiments, the furanosyl sugar moiety is a ribosyl moiety.

As used herein, the term “bicyclic nucleoside” (“BNA”) is a nucleoside comprising a bicyclic sugar.

As used herein, the term “BCL11 Transcription Factor A” or “BCL11A” or “Transcription factor B-cell lymphoma/leukemia 11A” refers to the C2H2 type zinc-finger transcription factor that functions, inter alia, in the repression of fetal hemoglobin and the switch from fetal hemoglobin to adult hemoglobin. The mRNA sequence of a reference hBCL11A gene (isoform 1) is set forth in SEQ ID NO: 1 (NCBI Ref.: NM_022893.4). The amino acid sequence of a reference hBCL11A protein (isoform 1) is set forth in SEQ ID NO: 3 (NCBI Ref.: NP_075044.2). The term BCL11A includes naturally occurring variants of BCL11A. BCL11A gene and mRNA sequences of e.g., human, mouse, rat, non-human primate (e.g., rhesus macaque, Macaca fascicularis (cynomolgus monkey)), are readily available through publicly available databases, including, e.g., GenBank, UniProt, OMIM, and the Macaca genome project web site.

As used herein, the term “blunt end” refers to a dsRNA molecule that does not contain any unpaired nucleotides at the end (e.g., 3′ terminus, 5′ terminus) of the dsRNA molecule (i.e., no nucleotide overhang(s)). The dsRNA molecule can have, for example, a blunt end at the 3′ end, 5′ end, or both the 3′ and 5′ end of the molecule.

As used herein, the term “CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), the entire contents of each of which is incorporated herein by reference for all purposes. Unless otherwise specified, the term “CDR” is a CDR as defined by Kabat et al., J. Biol. Chem. 252, 6609-6616 (1977) and Kabat et al., Sequences of protein of immunological interest. (1991). A person of ordinary skill in the art would be able to determine the CDRs as defined by another scheme, e.g., Chothia, IMGT, using ordinary methods known in the art.

The terms “CH1” and “CH1 region” are used interchangeably herein and refer to the first constant region of an immunoglobulin heavy chain. The amino acid sequence of an exemplary reference hIgG1 CH1 region is set forth in SEQ ID NO: 3461.

The terms “CH2” and “CH2 region” are used interchangeably herein and refer to the second constant region of an immunoglobulin heavy chain. The amino acid sequence of an exemplary reference hIgG1 CH2 region is set forth in SEQ ID NO: 3463.

The terms “CH3” and “CH3 region” are used interchangeably herein and refer to the third constant region of an immunoglobulin heavy chain. The amino acid sequence of an exemplary reference hIgG1 CH3 region is set forth in SEQ ID NO: 3464.

As used herein, the term “complementary” in reference to a first nucleotide sequence (e.g., a sense strand or a target mRNA) in relation to a second nucleotide sequence (e.g., an antisense strand), refers to the ability of a nucleic acid molecule comprising the first nucleotide sequence to hybridize to a nucleic acid molecule comprising the second nucleotide sequence and form a double stranded region (through base pair hydrogen bonds) under suitable in vivo or vitro conditions (e.g., under certain standard conditions, under mammalian (e.g., human) physiological conditions). A person of ordinary skill in the art would be able to select the set of conditions most appropriate for a hybridization test. Complementary sequences include, e.g., Watson-Crick base pairs. For example, complementary nucleobase pairs include adenine (A) and thymine (T); adenine (A) and uracil (U); and cytosine (C) and guanine (G). Complementary nucleobase pairs include natural and modified nucleotides, and nucleotide mimics, at least to the extent that the above hybridization requirements are fulfilled. As such, determinations of complementarity (as described herein) are independent of nucleotide chemical modifications (e.g., as described herein). For example, (C) and 5-methyl cytosine (mC) are both complementary to (G).

As used herein, the term “conjugation” refers to chemical conjugation of an agent (e.g., a nucleic acid molecule) with a moiety (e.g., carbohydrate, small molecule, polypeptide (e.g., antibody), polynucleotide, lipid, synthetic polymer (e.g., polymers of polyethylene glycol (PEG)), etc.). The moiety can be directly connected to the agent (e.g., nucleic acid molecule) or indirectly connected through a linker, e.g., as described herein. Chemical conjugation methods are well known in the art, as are commercially available conjugation reagents and kits, with detailed instructions for their use readily available from the commercial suppliers.

The terms “constant region” and “constant domain” are used interchangeably herein and refer to a carboxyl terminal portion of a light and/or heavy chain of a full-length antibody which is not directly involved in binding of an antibody to antigen, but which can exhibit various effector functions, such as interaction with an Ig Fc receptor (e.g., Fc gamma receptor). The constant region of an Ig molecule generally has a more conserved amino acid sequence relative to an Ig variable domain.

As used herein, the term “differing by no more than X nucleotides” in reference to a nucleotide sequence means that the nucleotide sequence comprises no more than X (wherein X is a specified number (e.g., 3, 2, 1, 0)) nucleotide variations (as defined herein) relative to a reference sequence. For example, the phrase “wherein the nucleotide sequence of the antisense strand differs by no more than 3 nucleotides from the nucleotide sequence of SEQ ID NO: X” means that the nucleotide sequence comprises no more than 3 nucleotide variations relative to the nucleotide sequence set forth in the cited SEQ ID NO: X.

As used herein, the term “disease” refers to any abnormal condition that impairs physiological function. The term is used broadly to encompass any disorder, illness, abnormality, pathology, sickness, condition, or syndrome in which physiological function is impaired, irrespective of the nature of the etiology. The term disease includes infection (e.g., a viral, bacterial, fungal, protozoal infection).

As used herein, the term “double stranded RNA agent” or “dsRNA agent” refers to a complex of two RNA molecules comprising a double stranded region comprising two anti-parallel and at least partially (e.g., substantially, fully) complementary nucleic acid sequences that form the double stranded region. For example, in some embodiments, the dsRNA agent comprises a sense strand and an antisense strand.

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

The term “effector function” when used in reference to an Ig Fc region or a protein comprising an Ig Fc region (e.g., a full-length antibody) refers to those biological activities attributable to the Ig Fc region of a typical full-length antibody, which therefore vary with the antibody isotype. Antibody effector functions include, but are not limited to, antibody-dependent cell-mediated cytotoxicity (ADCC), antibody-dependent cellular phagocytosis (ADCP), complement dependent cytotoxicity (CDC), Fc receptor binding (e.g., FcγRI, FcγRIIa, FcγRIIc, FcγRIIIa, and/or FcγRIIIb (e.g., FcγRI, FcγIIa, and/or FcγIIIa)), and Clq binding.

As used herein, the term “erythroid precursor cell” refers to any precursor of a mature erythrocyte (i.e., a mature enucleated red blood cell). As such, erythroid precursor cells include, but are not limited to, megakaryocyte erythroid progenitor cells, proerythroblast cells, early erythroblast cells, intermediate erythroblast cells, late erythroblast cells, and reticulocytes. In some embodiments, erythroid precursor cells include megakaryocyte erythroid progenitor cells, proerythroblast cells, early erythroblast cells, intermediate erythroblast cells, and late erythroblast cells. In some embodiments, erythroid precursor cells include proerythroblast cells, early erythroblast cells, intermediate erythroblast cells, and late erythroblast cells.

As used herein, the term “erythroid precursor cell targeting agent” refers to an agent that specifically binds to an antigen expressed on an erythroid precursor cell (or a subset thereof). For example, the antigen expressed in or on the erythroid precursor cell may be a membrane protein, for example an integral membrane protein or a peripheral membrane protein. Typically, an erythroid precursor cell targeting agent specifically binds to an antigen on the erythroid precursor cell that facilitates internalization of the erythroid precursor cell targeting agent (and any associated agent (e.g., an antisense strand, sense strand, dsRNA agent, RNAi agent, ASO, ssRNA agent, etc. described herein)) into the erythroid precursor cell. In some embodiments, an erythroid precursor cell targeting agent specifically binds to an internalizing, cell surface receptor on the erythroid precursor cell and is capable of being internalized into the erythroid precursor cell through receptor mediated internalization. In some embodiments, the erythroid precursor cell targeting agent is a protein (e.g., antibody), a peptide, a nucleic acid (e.g., an aptamer), or small molecule. In some embodiments, the erythroid precursor cell targeting agent is linked to an agent (e.g., an antisense strand, sense strand, dsRNA agent, RNAi agent, ASO, ssRNA agent, etc. described herein).

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, the entire contents of each of which is incorporated herein by reference for all purposes.

As used herein, the term “Fab” refers to an antigen binding domain that comprises a Fab heavy chain that comprises from N- to C-terminus a VH region and a CH1 region; and a light chain comprising from N- to C-terminus a VL region and a CL region; and wherein the Fab heavy chain and the light chain associate to form an antigen binding domain.

The term “Fab-Fc” as used herein refers to an antibody that comprises a Fab operably linked to an Fc region.

As used herein, the term “Fc region” refers to the C-terminal region of a Ig (e.g., a human Ig) heavy chain that comprises from N- to C-terminus at least a CH2 region operably connected to a CH3 region. In some embodiments, the Fc region comprises an Ig hinge region or at least a portion of an Ig hinge region operably connected to the N-terminus of the CH2 region. In some embodiments, the Fc region is engineered relative to a reference Fc region (e.g., comprises one or more amino acid modification), e.g., described herein. Additional examples of proteins with engineered Fc regions can be found in Saunders 2019 (K. O. Saunders, “Conceptual Approaches to Modulating Antibody Effector Functions and Circulation Half-Life,” 2019, Frontiers in Immunology, V. 10, Art. 1296, pp. 1-20, the entire contents of which is incorporated herein by reference for all purposes).

As used herein, the terms “first” and “second” with respect to Fc regions etc., are used for convenience of distinguishing when there is more than one of each type of moiety. Use of these terms is not intended to confer a specific order or orientation in the protein unless explicitly so stated. For example, an antibody described herein (e.g., in the case of a full-length antibody) may contain two Fc regions that associate e.g., via one or more covalent (e.g., disulfide) bond.

As used herein, the term “framework region” or “FR region” refers to the amino acid residues that are part of the variable region of an antibody, but are not part of the CDRs (e.g., using the Kabat definition of CDRs).

As used herein, the term “full-length antibody” refers to an antibody having a structure substantially similar to a native antibody structure (i) a first Ig light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer. In some embodiments, the two heavy chains comprise a substantially identical amino acid sequence; and the two light chains comprise a substantially identical amino acid sequence. In some embodiments, the two heavy chains comprise a substantially identical amino acid sequence except for one or more amino acid modifications that promote heterodimerization of the correct heavy chains (e.g., as described herein); and the two light chains comprise a substantially identical amino acid sequence. Antibody chains may be substantially identical but not entirely identical if they differ due to post-translational modifications, such as C-terminal cleavage of lysine residues, alternative glycosylation patterns, etc.

As used herein, the term “fully complementary” means that in a hybridized pair of a first nucleic acid molecule and a second nucleic acid molecule, 100% (all), of the bases in a contiguous sequence of the first nucleic acid molecule will hybridize with the same number of bases in a contiguous sequence of the second nucleic acid molecule. The contiguous sequence may comprise all or a part of the first and/or second nucleic acid molecule.

As used herein, the term “fully complementary” means that in a hybridized pair of a first nucleic acid molecule and a second nucleic acid molecule, 100% (all), of the bases in a contiguous sequence of the first nucleic acid molecule will hybridize with the same number of bases in a contiguous sequence of the second nucleic acid molecule. The contiguous sequence may comprise all or a part of the first and/or second nucleic acid molecule.

The term “functional variant” as used herein in reference to a protein refers to a protein that comprises at least one but no more than 20%, not more than 15%, not more than 12%, no more than 10%, no more than 8% amino acid variation (e.g., substitution, deletion, addition) compared to the amino acid sequence of a reference protein, wherein the protein retains at least one particular function of the reference protein. Not all functions of the reference protein (e.g., wild type) need be retained by the functional variant of the protein. In some instances, one or more functions are selectively reduced or eliminated. In some embodiments, the reference protein is a wild type protein.

The term “functional fragment” as used herein in reference to a protein refers to a fragment of a reference protein that retains at least one particular function. Not all functions of the reference polypeptide or protein need be retained by a functional fragment of the protein. In some instances, one or more functions are selectively reduced or eliminated. In some embodiments, the reference protein is a wild type protein.

As used herein, the term “fuse” and grammatical equivalents thereof refer to the operable connection of at least a one polypeptide derived from a first polypeptide to another polypeptide derived from a second polypeptide, wherein the first and second polypeptides are different. The term fuse encompasses both a direct connection of the at least two polypeptides through a peptide bond, and the indirect connection through a linker (e.g., a peptide linker).

As used herein, the term “fusion protein” and grammatical equivalents thereof refers to a protein that comprises at least one polypeptide derived from a first polypeptide operably connected to another polypeptide derived from a second polypeptide, wherein the first and second polypeptides are wherein the first and second polypeptides are not naturally found operably connected together. The at least two polypeptides of the fusion protein can be directly operably connected through a peptide bond; or can be indirectly operably connected through a linker (e.g., a peptide linker). Therefore, for example, the term fusion protein encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B). In some embodiments, the first polypeptide and the second polypeptide are different.

As used herein, the term “half-life extension moiety” refers to a moiety (e.g., small molecule, polypeptide, polynucleotide, carbohydrate, lipid, synthetic polymer (e.g., polymers of PEG), etc.) that when conjugated or otherwise operably connected (e.g., fused) to a polypeptide or protein (the subject polypeptide or protein), increases the half-life of the subject polypeptide or protein in vitro when administered to a subject (e.g., a human subject). The pharmacokinetic properties of the polypeptide or protein can be evaluated utilizing in vitro models known in the art.

As used herein, the term “half-life extension polypeptide” refers to a polypeptide that when operably connected to another polypeptide (the subject polypeptide), increases the half-life of the subject polypeptide in vitro when administered to a subject (e.g., a human subject). The pharmacokinetic properties of the polypeptide or protein can be evaluated utilizing in vitro models known in the art.

As used herein, the term “heavy chain” refers to the portion of an immunoglobulin (e.g., a human Ig) that typically comprises from N- to C-terminus a heavy chain variable region (VH), a CH1 region, a hinge region, a CH2 region, and a CH3 region. The constant regions of the heavy chain (i.e., the CH1 region, the hinge region, the CH2 region, and the CH3 region) can be any distinct isotype, for example, human alpha (α), delta (δ), epsilon (ε), gamma (γ), and mu (μ), based on the amino acid sequence of the constant domain, which give rise to the hIgA, hIgD, IgE, hIgG, and hIgM classes of human antibodies, respectively, including subclasses of hIgG, e.g., hIgG₁, hIgG₂, hIgG₃, and hIgG₄. As used herein, the term “heavy chain” when used in reference to a human antibody can refer to any distinct type, e.g., alpha (α), delta (δ), epsilon (ε), gamma (γ), and mu (μ), based on the amino acid sequence of the constant domain, which give rise to human IgA, IgD, IgE, IgG, and IgM classes of antibodies, respectively, including subclasses of human IgG, e.g., IgG₁, IgG₂, IgG₃, and IgG₄.

As used herein, the term “hematopoietic cell” refers to any blood cell. As such, the term hematopoietic cell, includes, but is not limited to, hematopoietic pluripotent stem cells (HSPCs), common myeloid progenitor cells, megakaryocyte erythroid progenitor cells, erythroid progenitor cells, proerythroblast cells, early erythroblast cells, intermediate erythroblast cells, late erythroblast cells, reticulocytes, megakaryocytes, platelets, granulocyte monocyte progenitor cells, monoblasts, promonocytes, monocytes, macrophages, myeloblasts, promyelocytes, myelocytes, eosinophils, basophils, neutrophils, common lymphoid progenitor cells, pro-NK lymphoblasts, NK cells, pro-B lymphoblasts, B lymphocytes, pro-T lymphoblasts, T lymphoblasts, and plasma cells.

As used herein, the term “hematopoietic cell targeting agent” refers to an agent that specifically binds to an antigen expressed on a hematopoietic cell (or a subset thereof). For example, the antigen expressed in or on the hematopoietic cell may be a membrane protein, for example an integral membrane protein or a peripheral membrane protein. Typically, a hematopoietic cell targeting agent specifically binds to an antigen on the hematopoietic cell that facilitates internalization of the hematopoietic cell targeting agent (and any associated agent (e.g., an antisense strand, sense strand, dsRNA agent, RNAi agent, ASO, ssRNA agent, etc. described herein)) into the hematopoietic cell. In some embodiments, a hematopoietic cell targeting agent specifically binds to an internalizing, cell surface receptor on the hematopoietic cell and is capable of being internalized into the hematopoietic cell through receptor mediated internalization. In some embodiments, the hematopoietic cell targeting agent is a protein (e.g., antibody), a peptide, a nucleic acid (e.g., an aptamer), or small molecule. In some embodiments, the hematopoietic cell targeting agent is linked to an agent (e.g., an antisense strand, sense strand, dsRNA agent, RNAi agent, ASO, ssRNA agent, etc. described herein).

As used herein, the term “heterologous,” when used to describe a first element in reference to a second element means that the first element and second element do not exist in nature disposed as described. For example, a nucleic acid molecule comprising a “heterologous moiety” means a nucleic acid molecule that is joined to a moiety (e.g., carbohydrate, small molecule, polypeptide, polynucleotide, lipid, synthetic polymer (e.g., polymers of PEG), etc.) that is not joined to the nucleic acid molecule in nature.

The terms “hinge” or “hinge region” are used interchangeably herein and refer to the hinge region of an immunoglobulin heavy chain. The amino acid sequence of an exemplary reference hIgG1 hinge region is set forth in SEQ ID NO: 3462.

As used herein, the term “isolated” with reference to a polypeptide, protein, or polynucleotide refers to a polypeptide, protein, or polynucleotide that is substantially free of other cellular components with which it is associated in the natural state.

As used herein, the term “nucleotide variation,” “variant nucleotide,” or use of the term “variation” and the like in reference to a nucleotide or nucleic acid sequence refers to a nucleic acid molecule that comprises at least one substitution, addition, deletion, or inversion of one or more nucleotide compared to a reference nucleic acid molecule. As used herein, the term “variant” or “variation” with reference to a peptide or protein refers to a peptide or protein that comprises at least one substitution, addition, deletion, or inversion of an amino acid residue compared to a reference peptide or protein.

As used herein, the term “modified agent” when used in reference to a nucleic acid molecule-based agent (or any component thereof (e.g., any nucleic acid molecule thereof)) (e.g., described herein, e.g., an antisense strand, a sense strand, a dsRNA agent, RNAi agent, an antisense oligonucleotide, etc.), refers to a nucleic acid molecule based agent (or any component thereof (e.g., any nucleic acid molecule thereof)) (e.g., described herein, e.g., an antisense strand, a sense strand, a dsRNA agent, RNAi agent, an antisense oligonucleotide, etc.) that comprises one or more modified nucleotide (as defined herein).

As used herein, the term “modified nucleotide,” “nucleotide modification,” or use of the term “modification” and the like in reference to a nucleotide or nucleic acid sequence refers to a nucleotide comprising a chemical modification, e.g., a modified sugar moiety, a modified nucleobase, and/or a modified internucleoside linkage, or any combination thereof. Exemplary modifications are provided herein, see, e.g., §§ 4.4, 4.4.1. In certain embodiments of the instant disclosure, inclusion of a deoxynucleotide—which is acknowledged as a naturally occurring form of nucleotide—if present within a nucleic acid molecule-based agent or component thereof (e.g., described herein, e.g., a sense strand, an antisense strand, a dsRNA agent, ssRNA agent, an antisense oligonucleotide, etc.) is considered to constitute a modified nucleotide.

As used herein, the term “nucleotide overhang” refers to at least one unpaired nucleotide that extends from the double stranded region of a nucleic acid molecule (e.g., a dsRNA molecule (e.g., a dsRNA molecule described herein)). For example, when a 3′-end of one strand of a dsRNA extends beyond the 5′-end of the other strand, or vice versa, there is a nucleotide overhang.

As used herein, the term, “non-complementary nucleotide mismatch” refers to a nucleotide within a region of complementarity (as described herein) that is not complementary to the corresponding nucleotide in the target nucleic acid molecule.

As used herein, the term “operably connected” refers to the linkage of two moieties in a functional relationship. For example, a polypeptide is operably connected to another polypeptide when they are linked (either directly or indirectly via a peptide linker) in frame such that both polypeptides are functional (e.g., a fusion protein described herein). Or for example, a transcription regulatory polynucleotide e.g., a promoter, enhancer, or other expression control element is operably linked to a polynucleotide that encodes a protein if it affects the transcription of the polynucleotide that encodes the protein. The term “operably connected” can also refer to the conjugation of a moiety to e.g., a polynucleotide or polypeptide (e.g., the conjugation of a PEG polymer to a protein).

As used herein, “partially complementary” means that in a hybridized pair of a first nucleic acid molecule and a second nucleic acid molecule, at least 70%, but not all, of the bases in a contiguous sequence of the first nucleic acid molecule will hybridize with the same number of bases in a contiguous sequence of the second nucleic acid molecule. The contiguous sequence may comprise all or a part of a first or second nucleic acid molecule.

The determination of “percent identity” between two sequences (e.g., protein (amino acid sequences) or polynucleotide (nucleic acid sequences)) can be accomplished using a mathematical algorithm. Determinations of identity (as described herein) are independent of nucleotide chemical modifications (e.g., as described herein). For example, (mC) is identical to (C) for the purposes of determining identity. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance intended for use in contact with the tissues of human beings and/or non-human animals, and without excessive toxicity, irritation, allergic response, or other problem or complication, commensurate with a reasonable therapeutic benefit/risk ratio.

The terms “nucleic acid molecule” and “polynucleotide” are used interchangeably herein and refer to a polymer of DNA or RNA. The nucleic acid molecule can be single-stranded or double-stranded; contain natural, non-natural, or altered nucleotides; and contain a natural, non-natural, or altered internucleoside linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “plurality” means 2 or more (e.g., 3 or more, 4 or more, 5 or more, 6 or more, 7 or more, 9 or more, or 10 or more).

As used herein, the terms “protein” and “polypeptide” refers to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” and “protein” are used interchangeably herein. In some embodiments, the protein is folded into its three-dimensional structure. Where proteins are contemplated herein, it should be understood that proteins folded into their three-dimensional structure are also provided herein.

As used herein, the term “region of complementarity” refers to a portion of a first nucleic acid molecule comprising a nucleotide sequence that is at least partially complementary to the nucleotide sequence of at least a portion of a second nucleic acid molecule.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleoside linkages, such as a phosphoroamidate linkage or a phosphorothioate linkage, instead of the phosphodiester found between the nucleotides of an unmodified nucleic acid molecule.

As used herein, the term “RNAi agent” refers to an agent that contains one or more RNA molecules which can mediate the targeted cleavage of an RNA molecule (e.g., an mRNA molecule) via an RNA-induced silencing complex (RISC) pathway. The RNAi agent, is thereby capable of e.g., modulating, e.g., inhibiting, the expression of a target gene (e.g., BCL11A) in a cell, e.g., a cell within a subject, such as a mammalian subject. In some embodiments, the RNAi agent is a dsRNA agent comprising a sense strand and an antisense strand that form a double stranded region, wherein optionally the sense strand and the antisense strand each independently comprise or consist of from about 19-23 nucleotides.

The term “scFv” or “single chain variable fragment” refers to an antibody that comprises a VH region operably connected via a peptide linker to a VL region, wherein the VH and VL regions associate to specifically bind an antigen (e.g., form an antigen binding domain). In some embodiments, the scFv comprises from N- to C-terminus an VH region, a peptide linker, and an VL region. In some embodiments, the scFv comprises from N- to C-terminus an VL region, a peptide linker, and an VH region.

The term “(scFv)₂” as used herein refers to an antibody that comprises a first and a second scFv operably connected (e.g., via a peptide linker). The first and second scFv can specifically bind the same or different antigens. In some embodiments, the first and second scFv are operably connected by a peptide linker.

The term “scFv-Fc” as used herein refers to an antibody that comprises a scFv operably linked (e.g., via a peptide linker) to an Fc domain or subunit of an Fc domain. In some embodiments, a scFv is operably connected to only a first Fc domain of a first and a second Fc domain pair. In some embodiments, a first scFv is operably connected to a first Fc domain and a second scFv is operably connected to a second Fc domain of a first and second Fc domain pair.

The term “(scFv)₂-Fc” as used herein refers to a (scFv)₂ operably linked (e.g., via a peptide linker) to an Fc domain or a subunit of an Fc domain. In some embodiments, a (scFv)₂ is operably connected to only a first Fc domain of a first and a second Fc domain pair. In some embodiments, a first (scFv)₂ is operably connected to a first Fc domain and a second (scFv)₂ is operably connected to a second Fc domain of a first and second Fc domain pair.

As used herein, the term “sense strand” refers that is an RNA molecule that is part of an RNAi agent (e.g., described herein) or part of a dsRNA agent (e.g., described herein) (e.g., an siRNA agent described herein) that comprises a region that is at least partially (e.g., substantially, fully) complementary to a region of the antisense strand (as defined herein). The sense strand is often referred to as such with reference to the orientation of the sequence of the sense strand being the same with respect to a target RNA (e.g., mRNA sequence).

As used herein, the term “single domain antibody” or “sdAb” refers to an antibody having a single monomeric variable antibody domain. A sdAb is able to specifically bind to a specific antigen. A VHH (as defined herein) is an example of a sdAb.

As used herein, the term “specifically binds” refers to preferential interaction, i.e., significantly higher binding affinity, between a first protein (e.g., an antibody) and a second protein (e.g., an antigen) relative to other amino acid sequences. Herein, when a first protein is said to “specifically bind” to a second protein, it is understood that the first protein specifically binds to an epitope of the second protein. The term “epitope” refers to the portion of the second protein that the first protein specifically recognizes. The term specifically binds includes molecules that are cross reactive with the same epitope of a different species. For example, an antibody that specifically binds human TFR may be cross reactive with TFR of another species (e.g., cynomolgus, murine, etc.), and still be considered herein to specifically bind human TFR. A protein can specifically bind more than one different protein. Specific binding can be measured, e.g., through measuring binding affinity (e.g., using standard methods known in the art and described herein (e.g., surface plasmon resonance (SPR) (e.g., BIAcore®-based assay), a common method known in the art (see, e.g., Wilson, Science 295:2103, 2002; Wolff et al., Cancer Res. 55:2560, 1993; and U.S. Pat. Nos. 5,283,173, 5,468,614, the full contents of each of which are incorporated by reference herein for all purposes).

As used herein, the term “subject” includes any animal, such as a human or other animal. In some embodiments, the subject is a vertebrate animal (e.g., mammal, bird, fish, reptile, or amphibian). In some embodiments, the subject is a human. In some embodiments, the method subject is a non-human mammal. In some embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

As used herein, “substantially complementary” means that in a hybridized pair of a first nucleic acid molecule and a second nucleic acid molecule, at least 85%, but not all, of the bases in a contiguous sequence of the first nucleic acid molecule will hybridize with the same number of bases in a contiguous sequence of the second nucleic acid molecule. The contiguous sequence may comprise all or a part of a first or second nucleic acid molecule.

In some embodiments, the term “substantially all” means at least 95%, 96%, 97%, 98% or 99%, e.g., of the subject of said sentence. The term “substantially all” preferably excludes 100%. For example, in some embodiments, the term “substantially all of the nucleotides in the sense strand and/or antisense strand are modified” means that at least 95%, 96%, 97%, 98% or 99% of said nucleotides are modified. For example, in some embodiments, the term “substantially all of the nucleotides of the agent are modified” means that at least 95%, 96%, 97%, 98% or 99% of said nucleotides are modified. For example, in some embodiments, the term “substantially all of the nucleotides of the agent are unmodified” means that at least 95%, 96%, 97%, 98% or 99% of said nucleotides are unmodified. For example, in some embodiments the term “wherein the dsRNA agent is in the sodium salt form, sodium ions are present in the composition comprising the dsRNA agent as counterions for substantially all of the phosphodiester or phosphorothioate groups present in the dsRNA agent” means that wherein the dsRNA agent is in the sodium salt form, sodium ions are present in the composition comprising the dsRNA agent as counterions for at least 95%, 96%, 97%, 98% or 99% of the phosphodiester or phosphorothioate groups present in the dsRNA agent.

As used herein, the term “target nucleic acid sequence” refers to a contiguous portion of the nucleotide sequence of a nucleic acid sequence (e.g., an mRNA molecule formed during the transcription of a target gene (e.g., BCL11A)). In some embodiments, the target nucleic acid sequence is an mRNA molecule formed during the transcription of a target gene (e.g., BCL11A)). In some embodiments, the target nucleic acid molecule comprises an mRNA that is a product of RNA processing of a primary transcription product. The target portion of the sequence (e.g., mRNA) will be at least long enough to serve as a substrate for the agent described herein (e.g., RNA agents, RNAi agents, dsRNA agents (e.g., siRNA agents), ssRNA agents (e.g., antisense oligonucleotides)). For example, in some embodiments, the target portion of the sequence (e.g., mRNA) will be at least long enough to serve as a substrate for RNAi-directed cleavage at or near that portion of the nucleotide sequence of an mRNA molecule formed during the transcription of a BCL11A gene. For example, in some embodiments, the target portion of the sequence (e.g., mRNA) will be at least long enough to serve as a substrate for an antisense oligonucleotide. In one embodiment, the target sequence is within the protein coding region of BCL11A.

As used herein, the term “therapeutic agent” refers to an agent (e.g., a conjugate described herein) capable of achieving a desired therapeutic result in a subject or ex vivo (e.g., capable of treating a disease as defined herein) when administered at a therapeutically effective amount.

As used herein, the term “therapeutically effective amount” of a therapeutic agent refers to any amount of the therapeutic agent that, when used alone or in combination with another therapeutic agent, improves a disease condition, e.g., protects a subject against the onset of a disease (or infection); improves a symptom of disease or infection, e.g., decreases severity of disease or infection symptoms, decreases frequency or duration of disease or infection symptoms, increases disease or infection symptom-free periods; prevents or reduces impairment or disability due to the disease or infection; or promotes disease (or infection) regression. The ability of a therapeutic agent to improve a disease condition can be evaluated using a variety of methods known to the skilled practitioner, such as in human subjects during clinical trials, in animal model systems predictive of efficacy in humans, or by assaying the activity of the agent in in vitro assays.

As used herein, the term “transferrin” or “TF” refers to the plasma glycoprotein transferrin that functions, inter alia, in iron metabolism and the transport of iron through the blood to various tissues, such as the liver, spleen, and bone marrow. The amino acid sequence of a reference human TF (hTF) protein is set forth in SEQ ID NO: 3283 (UniProt Accession P02787).

As used herein, the term “transferrin receptor” or “TFR” refers to the transmembrane homodimeric glycoprotein that functions, inter alia, in the cellular uptake of iron from the plasma glycoprotein transferrin. The term TFR includes, where applicable, multiple isoforms and homologs. For example, human TFR (hTFR) includes homologs hTFR1 and hTFR2. The amino acid sequence of a reference hTFR1 protein is set forth in SEQ ID NO: 3281 (UniProt Accession P02786|). TFR1 is also commonly known in the art as CD71. The terms TFR1 and CD71 are used interchangeably herein. The amino acid sequence of a reference hTFR2 protein is set forth in SEQ ID NO: 3282 (UniProt Accession Q9UP52).

As used herein, the terms “treat,” treating,” “treatment,” and the like refer to reducing or ameliorating a disease and/or symptom(s) associated therewith or obtaining a desired pharmacologic and/or physiologic effect. It will be appreciated that, although not precluded, treating a disease does not require that the disease, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease. To this end, the presently disclosed methods comprise administering a therapeutically effective amount of a compositions as described herein.

As used herein, the term “variation” or “variant” or use the like in reference to a nucleotide or nucleic acid sequence refers to a nucleic acid molecule that comprises at least one substitution, addition, deletion, or inversion of one or more nucleotide compared to a reference nucleic acid molecule. Likewise, as used herein, the term “variation” or “variant” or use the like with reference to a peptide or protein refers to a peptide or protein that comprises at least one substitution, addition, deletion, or inversion of an amino acid residue compared to a reference peptide or protein.

A “variation that promotes heterodimerization of a first Fc region and a second Fc region” (or similar phrasing) is a manipulation of the peptide backbone or the post-translational modifications of an Fc region that reduces or prevents the association of a polypeptide comprising the Fc region with an identical polypeptide to form a homodimer. A modification promoting association as used herein particularly includes separate modifications made to each of the two Fc regions desired to associate (i.e., a first Fc region and a second Fc region), wherein the modifications are complementary to each other so as to promote association of the two Fc regions. For example, a modification promoting association may alter the structure or charge of one or both of the Fc regions so as to make their association sterically or electrostatically favorable, respectively. Thus, heterodimerization occurs between a polypeptide comprising the first Fc region and a polypeptide comprising the second Fc region, which might be non-identical in the sense that further components fused to each of the Fc regions (e.g., antigen binding domains) are not the same. In some embodiments the modification promoting association comprises an amino acid mutation in the Fc region, specifically an amino acid substitution. In a particular embodiment, the modification promoting association comprises a separate amino acid mutation, specifically one or more amino acid substitution, in each of the first Fc region and the second Fc region (e.g., described herein).

As used herein, the term “variable region” refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL region” are used interchangeably to refer to an immunoglobulin light chain variable region. A VL region can be incorporated into an antibody, e.g., a scFv, a Fab, a full-length antibody. For example, a scFv comprises a VL region operably connected via a peptide linker to a VH region.

The terms “VH” and “VH region” are used interchangeably to refer to an immunoglobulin heavy chain variable region. A VH region can be incorporated into an antibody, e.g., a scFv, a Fab, a full-length antibody. For example, a scFv comprises a VH region operably connected via a peptide linker to a VL region.

The term “VHH” as used herein refers to a type of single domain antibody (sdAb) that has a single monomeric heavy chain variable antibody domain (VH). Such antibodies can be found in or produced from camelid mammals (e.g., camels, llamas) which are naturally devoid of light chains or synthetically produced.

The term “(VHH)₂” as used herein refers to an antibody that comprises a first and a second VHH operably connected (e.g., via a peptide linker). The first and the second VHH can specifically bind the same or different antigens. In some embodiments, the first and second VHH are operably connected by a peptide linker.

The term “VHH-Fc” as used herein refers to an antibody that comprises a VHH operably linked (e.g., via a peptide linker) to an Fc domain or a subunit of an Fc domain. In some embodiments, a VHH is operably connected to only a first Fc domain of a first and a second Fc domain pair. In some embodiments, a first VHH is operably connected to a first Fc domain and a second VHH is operably connected to a second Fc domain of a first Fc and a second Fc pair.

The term “(VHH)₂-Fc” as used herein refers to (VHH)₂ operably linked (e.g., via a peptide linker) to an Fc domain or a subunit of an Fc domain. In some embodiments, a (VHH)₂ is operably connected to only a first Fc domain of a first and a second Fc domain pair. In some embodiments, a first (VHH)₂ is operably connected to a first Fc domain and a second (VHH)₂ is operably connected to a second Fc domain of a first Fc and a second Fc pair.

### 4.2 RNAi Agents

Provided herein are, inter alia, agents (e.g., RNAi agents, dsRNA agents), useful in, inter alia, inhibiting expression of BCL11 transcription factor A (BCL11A) (e.g., human BCL11A (hBCL11A)) (e.g., within a cell, e.g., within a cell in a subject, e.g., a mammalian subject, e.g., a human subject) (e.g., through the degradation of BCL11A (e.g., hBCL11A) mRNA).

BCL11A is a transcription factor that e.g., represses transcription of a range of genes involved in e.g., cell proliferation and differentiation. BCL11A has several isoforms, including, BCL11A-XL, BCL11A-L, BCL11A-S, and BCL11A-XS. See, e.g., Seigfried, F. A.; Britsch, S. The Role of Bcl11 Transcription Factors in Neurodevelopmental Disorders. Biology 2024, 13, 126. https://doi.org/10.3390/biology13020126. It should be understood that the agents described herein (e.g., dsRNA agents and antisense oligonucleotides) can target any one of or any combination of or all of the isoforms of BCL11A.

The mRNA sequence of a reference hBCL11A (isoform 1) gene is set forth in SEQ ID NO: 1 (NCBI Ref.: NM_022893.4). The reverse complement sequence of the hBCL11A mRNA (isoform 1) is set forth in SEQ ID NO: 2. The amino acid sequence of a reference hBCL11A (isoform 1) protein is set forth in SEQ ID NO: 3 (NCBI Ref.: NP_075044.2). See Table 1, herein.

TABLE 1
The mRNA and Amino Acid Sequence of a Reference hBCL11A Protein.

| | | SEQ ID |
|---|---|---|
| Description | Amino Acid Sequence | NO |
| hBCL11A | GUCUCUGUCCAUCCAGACUCCUGACGUUCAAGUUCGCAGGGACGUCAC | 1 |
| mRNA | GUCCGCACUUGAACUUGCAGCUCAGGGGGGCUUUUGCCAUUUUUUUCA | |
| Transcript | UCUCUCUCUCUCUCUCUCCCUCUAUCUCUCUUCUCUCUCUCUCCCUCU | |
| Variant 1 | UUUUUUUUUUUUUUUUUUUUUUUUUUUUGCUUAAAAAAAAGCCAUGAC | |
| NCBI Ref.: | GGCUCUCCCACAAUUCAUCUUCCCUGCGCCAUCUUUGUAUUAUUUCUA | |
| NM_022893.4 | AUUUAUUUUGGAUGUCAAAAGGCACUGAUGAAGAUAUUUUCUCUGGAG | |
| | CGCCCGCCGCCGCCGCCGCCGCCGCCGCCGCCCGCCCCGCAGCCCACC | |
| | UCUCCUUCUUUCUAACCCGGCUCUCCCGAUGUGAACCGAGCCGUCGUC | |
| | AUGUCUCGCCGCAAGCAAGGCAAACCCCAGCACUUAAGCAAACGGGAA | |
| | UUCUCGCCCGAGCCUCUUGAAGCCAUUCUUACAGAUGAUGAACCAGAC | |
| | CACGGCCCGUUGGGAGCUCCAGAAGGGGAUCAUGACCUCCUCACCUGU | |
| | GGGCAGUGCCAGAUGAACUUCCCAUUGGGGGACAUUCUUAUUUUUAUC | |
| | GAGCACAAACGGAAACAAUGCAAUGGCAGCCUCUGCUUAGAAAAAGCU | |
| | GUGGAUAAGCCACCUUCCCCUUCACCAAUCGAGAUGAAAAAAGCAUCC | |
| | AAUCCCGUGGAGGUUGGCAUCCAGGUCACGCCAGAGGAUGACGAUUGU | |
| | UUAUCAACGUCAUCUAGAGGAAUUUGCCCCAAACAGGAACACAUAGCA | |
| | GAUAAACUUCUGCACUGGAGGGGCCUCUCCUCCCCUCGUUCUGCACAU | |
| | GGAGCUCUAAUCCCCACGCCUGGGAUGAGUGCAGAAUAUGCCCCGCAG | |
| | GGUAUUUGUAAAGAUGAGCCCAGCAGCUACACAUGUACAACUUGCAAA | |
| | CAGCCAUUCACCAGUGCAUGGUUUCUCUUGCAACACGCACAGAACACU | |
| | CAUGGAUUAAGAAUCUACUUAGAAAGCGAACACGGAAGUCCCCUGACC | |
| | CCGCGGGUUGGUAUCCCUUCAGGACUAGGUGCAGAAUGUCCUUCCCAG | |
| | CCACCUCUCCAUGGGAUUCAUAUUGCAGACAAUAACCCCUUUAACCUG | |
| | CUAAGAAUACCAGGAUCAGUAUCGAGAGAGGCUUCCGGCCUGGCAGAA | |
| | GGGCGCUUUCCACCCACUCCCCCCCUGUUUAGUCCACCACCGAGACAU | |
| | CACUUGGACCCCCACCGCAUAGAGCGCCUGGGGGCGGAAGAGAUGGCC | |
| | CUGGCCACCCAUCACCCGAGUGCCUUUGACAGGGUGCUGCGGUUGAAU | |
| | CCAAUGGCUAUGGAGCCUCCCGCCAUGGAUUUCUCUAGGAGACUUAGA | |
| | GAGCUGGCAGGGAACACGUCUAGCCCACCGCUGUCCCCAGGCCGGCCC | |
| | AGCCCUAUGCAAAGGUUACUGCAACCAUUCCAGCCAGGUAGCAAGCCG | |
| | CCCUUCCUGGCGACGCCCCCCCUCCCUCCUCUGCAAUCCGCCCCUCCU | |
| | CCCUCCCAGCCCCCGGUCAAGUCCAAGUCAUGCGAGUUCUGCGGCAAG | |
| | ACGUUCAAAUUUCAGAGCAACCUGGUGGUGCACCGGCGCAGCCACACG | |
| | GGCGAGAAGCCCUACAAGUGCAACCUGUGCGACCACGCGUGCACCCAG | |
| | GCCAGCAAGCUGAAGCGCCACAUGAAGACGCACAUGCACAAAUCGUCC | |
| | CCCAUGACGGUCAAGUCCGACGACGGUCUCUCCACCGCCAGCUCCCCG | |
| | GAACCCGGCACCAGCGACUUGGUGGGCAGCGCCAGCAGCGCGCUCAAG | |
| | UCCGUGGUGGCCAAGUUCAAGAGCGAGAACGACCCCAACCUGAUCCCG | |
| | GAGAACGGGGACGAGGAGGAAGAGGAGGACGACGAGGAAGAGGAAGAA | |
| | GAGGAGGAAGAGGAGGAGGAGGAGCUGACGGAGAGCGAGAGGGUGGAC | |
| | UACGGCUUCGGGCUGAGCCUGGAGGCGGCGCGCCACCACGAGAACAGC | |
| | UCGCGGGGCGCGGUCGUGGGCGUGGGCGACGAGAGCCGCGCCCUGCCC | |
| | GACGUCAUGCAGGGCAUGGUGCUCAGCUCCAUGCAGCACUUCAGCGAG | |
| | GCCUUCCACCAGGUCCUGGGCGAGAAGCAUAAGCGCGGCCACCUGGCC | |
| | GAGGCCGAGGGCCACAGGGACACUUGCGACGAAGACUCGGUGGCCGGC | |
| | GAGUCGGACCGCAUAGACGAUGGCACUGUUAAUGGCCGCGGCUGCUCC | |
| | CCGGGCGAGUCGGCCUCGGGGGGCCUGUCCAAAAAGCUGCUGCUGGGC | |
| | AGCCCCAGCUCGCUGAGCCCCUUCUCUAAGCGCAUCAAGCUCGAGAAG | |
| | GAGUUCGACCUGCCCCCGGCCGCGAUGCCCAACACGGAGAACGUGUAC | |
| | UCGCAGUGGCUCGCCGGCUACGCGGCCUCCAGGCAGCUCAAAGAUCCC | |
| | UUCCUUAGCUUCGGAGACUCCAGACAAUCGCCUUUUGCCUCCUCGUCG | |
| | GAGCACUCCUCGGAGAACGGGAGUUUGCGCUUCUCCACACCGCCCGGG | |
| | GAGCUGGACGGAGGGAUCUCGGGGCGCAGCGGCACGGGAAGUGGAGGG | |
| | AGCACGCCCCAUAUUAGUGGUCCGGGCCCGGGCAGGCCCAGCUCAAAA | |
| | GAGGGCAGACGCAGCGACACUUGUGAGUACUGUGGGAAAGUCUUCAAG | |
| | AACUGUAGCAAUCUCACUGUCCACAGGAGAAGCCACACGGGCGAAAGG | |
| | CCUUAUAAAUGCGAGCUGUGCAACUAUGCCUGUGCCCAGAGUAGCAAG | |
| | CUCACCAGGCACAUGAAAACGCAUGGCCAGGUGGGGAAGGACGUUUAC | |
| | AAAUGUGAAAUUUGUAAGAUGCCUUUUAGCGUGUACAGUACCCUGGAG | |
| | AAACACAUGAAAAAAUGGCACAGUGAUCGAGUGUUGAAUAAUGAUAUA | |
| | AAAACUGAAUAGAGGUAUAUUAAUACCCCUCCCUCACUCCCACCUGAC | |
| | ACCCCCUUUUUCACCACUCCCCUUCCCCAUCGCCCUCCAGCCCCACUC | |
| | CCUGUAGGAUUUUUUUCUAGUCCCAUGUGAUUUAAACAAACAAACAAA | |
| | CAAACAGAAGUAACGAAGCUAAGAAUAUGAGAGUGCUUGUCACCAGCA | |
| | CACCUGUUUUUUUUCUUUUUCUUUUUCUUUUUUCUUUUUCCUUUUUUU | |
| | UUUUUUUCCUUUAUGUUCUCACCGUUUGAAUGCAUGAUCUGUAUGGGG | |
| | CAAUACUAUUGCAUUUUACGCAAACUUUGAGCCUUUCUCUUGUGCAAU | |
| | AAUUUACAUGUUGUGUAUGUUUUUUUUUAAACUUAGACAGCAUGUAUG | |
| | GUAUGUUAUGGCUAUUUUAAAUUGUCCCUAAUUCGUUGCUGAGCAAAC | |
| | AUGUUGCUGUUUCCAGUUCCGUUCUGAGAGAAAAAGAGAGAGAGAGAG | |
| | AAAAAGACCAUGCUGCAUACAUUCUGUAAUACAUAUCAUGUACAGUUU | |
| | UAUUUUAUAACGUGAGGAGGAAAAACAGUCUUUGGAUUAACCCUCUAU | |
| | AGACAGAAUAGAUAGCACUGAAAAAAAAUCUCUAUGAGCUAAAUGUCU | |
| | GUCUCUAAAGGGUUAAAUGUAUCAAUUGGAAAGGAAGAAAAAAGGCCU | |
| | UGAAUUGACAAAUUAACAGAAAAACAGAACAAGUUUAUUCUAUCAUUU | |
| | GGUUUUAAAAUAUGAGUGCCUUGGAUCUAUUAAAACCACAUCGAUGGU | |
| | UCUUUCUACUUGUUAUAAACUUGUAGCUUAAUUCAGCAUUGGGUGAGG | |
| | UAAUAAACCUUAGGAACUAGCAUAUAAUUCUAUAUUGUAUUUCUCACA | |
| | ACAAUGGCUACCUAAAAAGAUGACCCAUUAUGUCCUAGUUAAUCAUCA | |
| | UUUUUCCUUUAGUUUAAUUUUAUAAACAAAACUGAUUAUACCAGUAUA | |
| | AAAGCUACUUUGCUCCUGGUGAGAGCUUAAAAGAAAUGGGCUGUUUUG | |
| | CCCAAAGUUUUAUUUUUUUUAAACAAUGAUUAAAUUGAAUGUGUAAUG | |
| | UGCAAAAGCCCUGGAACGCAAUUAAAUACACUAGUAAGGAGUUCAUUU | |
| | UAUGAAGAUAUUUGCUUUAAUAAUGUCUUUUUAAAAAUACUGGCACCA | |
| | AAAGAAAUAGAUCCAGAUCUACUUGGUUGUCAAGUGGACAAUCAAAUG | |
| | AUAAACUUUAAGACCUUGUAUACCAUAUUGAAAGGAAGAGGCUGACAA | |
| | UAAGGUUUGACAGAGGGGAACAGAAGAAAAUAAUAUGAUUUAUUAGCA | |
| | CAACGUGGUACUAUUUGCCAUUUAAAACUAGAACAGGUAUAUAAGCUA | |
| | AUAUUGAUACAAUGAUGAUUAACUAUGAAUUCUUAAGACUUGCAUUUA | |
| | AAUGUGACAUUCUUAAAAAAAGAAGAGAAAGAAUUUUAAGAGUAGCAG | |
| | UAUAUAUGUCUGUGCUCCCUAAAAGUUGUACUUCAUUUCUUUUCCAUA | |
| | CACUGUGUGCUAUUUGUGUUAACAUGGAAGAGGAUUCAUUGUUUUUAU | |
| | UUUUAUUUUUUUAAUUUUUUCUUUUUUAUUAAGCUAGCAUCUGCCCCA | |
| | GUUGGUGUUCAAAUAGCACUUGACUCUGCCUGUGAUAUCUGUAUCUUU | |
| | UCUCUAAUCAGAGAUACAGAGGUUGAGUAUAAAAUAAACCUGCUCAGA | |
| | UAGGACAAUUAAGUGCACUGUACAAUUUUCCCAGUUUACAGGUCUAUA | |
| | CUUAAGGGAAAAGUUGCAAGAAUGCUGAAAAAAAAUUGAACACAAUCU | |
| | CAUUGAGGAGCAUUUUUUAAAAACUAAAAAAAAAAAAACUUUGCCAGC | |
| | CAUUUACUUGACUAUUGAGCUUACUUACUUGGACGCAACAUUGCAAGC | |
| | GCUGUGAAUGGAAACAGAAUACACUUAACAUAGAAAUGAAUGAUUGCU | |
| | UUCGCUUCUACAGUGCAAGGAUUUUUUUGUACAAAACUUUUUUAAAUA | |
| | UAAAUGUUAAGAAAAAUUUUUUUUAAAAAACACUUCAUUAUGUUUAGG | |
| | GGGGAACUGCAUUUUAGGGUUCCAUUGUCUUGGUGGUGUUACAAGACU | |
| | UGUUAUCCAUUUAAAAAUGGUAGUGGAAAUUCUAUGCCUUGGAUACAC | |
| | ACCGCUCUUCAGGUUGUAAAAAAAAAAAACAUACAUUGGGGAAAGGUU | |
| | UAAGAUUAUAUAGUACUUAAAUAUAGGAAAAUGCACACUCAUGUUGAU | |
| | UCCUAUGCUAAAAUACAUUUAUGGUCUUUUUUCUGUAUUUCUAGAAUG | |
| | GUAUUUGAAUUAAAUGUUCAUCUAGUGUUAGGCACUAUAGUAUUUAUA | |
| | UUGAAGCUUGUAUUUUUAACUGUUGCUUGUUCUCUUAAAAGGUAUCAA | |
| | UGUACCUUUUUUGGUAGUGGAAAAAAAAAAGACAGGCUGCCACAGUAU | |
| | AUUUUUUUAAUUUGGCAGGAUAAUAUAGUGCAAAUUAUUUGUAUGCUU | |
| | CAAAAAAAAAAAAAAGAGAGAAACAAAAAAGUGUGACAUUACAGAUGA | |
| | GAAGCCAUAUAAUGGCGGUUUGGGGGAGCCUGCUAGAAUGUCACAUGG | |
| | AUGGCUGUCAUAGGGGUUGUACAUAUCCUUUUUUGUUCCUUUUUCCUG | |
| | CUGCCAUACUGUAUGCAGUACUGCAAGCUAAUAACGUUGGUUUGUUAU | |
| | GUAGUGUGCUUUUUGUCCCUUUCCUUCUAUCACCCUACAUUCCAGCAU | |
| | CUUACCUUCAUAUGCAGUAAAAGAAAGAAAGAAAAAAAAAGGAAAAAA | |
| | AAAAAAAAACCAAUGUUUUGCAGUUUUUUUCAUUGCCAAAAACUAAAU | |
| | GGUGCUUUAUAUUUAGAUUGGAAAGAAUUUCAUAUGCAAAGCAUAUUA | |
| | AAGAGAAAGCCCGCUUUAGUCAAUACUUUUUUGUAAAUGGCAAUGCAG | |
| | AAUAUUUUGUUAUUGGCCUUUUCUAUUCCUGUAAUGAAAGCUGUUUGU | |
| | CGUAACUUGAAAUUUUAUCUUUUACUAUGGGAGUCACUAUUUAUUAUU | |
| | GCUUAUGUGCCCUGUUCAAAACAGAGGCACUUAAUUUGAUCUUUUAUU | |
| | UUUCUUUGUUUUUAUUUUUUUUUUUAUUUAGAUGACCAAAGGUCAUUA | |
| | CAACCUGGCUUUUUAUUGUAUUUGUUUCUGGUCUUUGUUAAGUUCUAU | |
| | UGGAAAAACCACUGUCUGUGUUUUUUUGGCAGUUGUCUGCAUUAACCU | |
| | GUUCAUACACCCAUUUUGUCCCUUUAUUGAAAAAAUAAAAAAAAUUAA | |
| | AGUACA | |
| Reverse | UGUACUUUAAUUUUUUUUAUUUUUUCAAUAAAGGGACAAAAUGGGUGU | 2 |
| Complement of | AUGAACAGGUUAAUGCAGACAACUGCCAAAAAAACACAGACAGUGGUU | |
| hBCL11A | UUUCCAAUAGAACUUAACAAAGACCAGAAACAAAUACAAUAAAAAGCC | |
| mRNA | AGGUUGUAAUGACCUUUGGUCAUCUAAAUAAAAAAAAAAAUAAAAACA | |
| | AAGAAAAAUAAAAGAUCAAAUUAAGUGCCUCUGUUUUGAACAGGGCAC | |
| | AUAAGCAAUAAUAAAUAGUGACUCCCAUAGUAAAAGAUAAAAUUUCAA | |
| | GUUACGACAAACAGCUUUCAUUACAGGAAUAGAAAAGGCCAAUAACAA | |
| | AAUAUUCUGCAUUGCCAUUUACAAAAAAGUAUUGACUAAAGCGGGCUU | |
| | UCUCUUUAAUAUGCUUUGCAUAUGAAAUUCUUUCCAAUCUAAAUAUAA | |
| | AGCACCAUUUAGUUUUUGGCAAUGAAAAAAACUGCAAAACAUUGGUUU | |
| | UUUUUUUUUUUUCCUUUUUUUUUCUUUCUUUCUUUUACUGCAUAUGAA | |
| | GGUAAGAUGCUGGAAUGUAGGGUGAUAGAAGGAAAGGGACAAAAAGCA | |
| | CACUACAUAACAAACCAACGUUAUUAGCUUGCAGUACUGCAUACAGUA | |
| | UGGCAGCAGGAAAAAGGAACAAAAAAGGAUAUGUACAACCCCUAUGAC | |
| | AGCCAUCCAUGUGACAUUCUAGCAGGCUCCCCCAAACCGCCAUUAUAU | |
| | GGCUUCUCAUCUGUAAUGUCACACUUUUUUGUUUCUCUCUUUUUUUUU | |
| | UUUUUGAAGCAUACAAAUAAUUUGCACUAUAUUAUCCUGCCAAAUUAA | |
| | AAAAAUAUACUGUGGCAGCCUGUCUUUUUUUUUUCCACUACCAAAAAA | |
| | GGUACAUUGAUACCUUUUAAGAGAACAAGCAACAGUUAAAAAUACAAG | |
| | CUUCAAUAUAAAUACUAUAGUGCCUAACACUAGAUGAACAUUUAAUUC | |
| | AAAUACCAUUCUAGAAAUACAGAAAAAAGACCAUAAAUGUAUUUUAGC | |
| | AUAGGAAUCAACAUGAGUGUGCAUUUUCCUAUAUUUAAGUACUAUAUA | |
| | AUCUUAAACCUUUCCCCAAUGUAUGUUUUUUUUUUUUACAACCUGAAG | |
| | AGCGGUGUGUAUCCAAGGCAUAGAAUUUCCACUACCAUUUUUAAAUGG | |
| | AUAACAAGUCUUGUAACACCACCAAGACAAUGGAACCCUAAAAUGCAG | |
| | UUCCCCCCUAAACAUAAUGAAGUGUUUUUUAAAAAAAAUUUUUCUUAA | |
| | CAUUUAUAUUUAAAAAAGUUUUGUACAAAAAAAUCCUUGCACUGUAGA | |
| | AGCGAAAGCAAUCAUUCAUUUCUAUGUUAAGUGUAUUCUGUUUCCAUU | |
| | CACAGCGCUUGCAAUGUUGCGUCCAAGUAAGUAAGCUCAAUAGUCAAG | |
| | UAAAUGGCUGGCAAAGUUUUUUUUUUUUUAGUUUUUAAAAAAUGCUCC | |
| | UCAAUGAGAUUGUGUUCAAUUUUUUUUCAGCAUUCUUGCAACUUUUCC | |
| | CUUAAGUAUAGACCUGUAAACUGGGAAAAUUGUACAGUGCACUUAAUU | |
| | GUCCUAUCUGAGCAGGUUUAUUUUAUACUCAACCUCUGUAUCUCUGAU | |
| | UAGAGAAAAGAUACAGAUAUCACAGGCAGAGUCAAGUGCUAUUUGAAC | |
| | ACCAACUGGGGCAGAUGCUAGCUUAAUAAAAAAGAAAAAAUUAAAAAA | |
| | AUAAAAAUAAAAACAAUGAAUCCUCUUCCAUGUUAACACAAAUAGCAC | |
| | ACAGUGUAUGGAAAAGAAAUGAAGUACAACUUUUAGGGAGCACAGACA | |
| | UAUAUACUGCUACUCUUAAAAUUCUUUCUCUUCUUUUUUUAAGAAUGU | |
| | CACAUUUAAAUGCAAGUCUUAAGAAUUCAUAGUUAAUCAUCAUUGUAU | |
| | CAAUAUUAGCUUAUAUACCUGUUCUAGUUUUAAAUGGCAAAUAGUACC | |
| | ACGUUGUGCUAAUAAAUCAUAUUAUUUUCUUCUGUUCCCCUCUGUCAA | |
| | ACCUUAUUGUCAGCCUCUUCCUUUCAAUAUGGUAUACAAGGUCUUAAA | |
| | GUUUAUCAUUUGAUUGUCCACUUGACAACCAAGUAGAUCUGGAUCUAU | |
| | UUCUUUUGGUGCCAGUAUUUUUAAAAAGACAUUAUUAAAGCAAAUAUC | |
| | UUCAUAAAAUGAACUCCUUACUAGUGUAUUUAAUUGCGUUCCAGGGCU | |
| | UUUGCACAUUACACAUUCAAUUUAAUCAUUGUUUAAAAAAAAUAAAAC | |
| | UUUGGGCAAAACAGCCCAUUUCUUUUAAGCUCUCACCAGGAGCAAAGU | |
| | AGCUUUUAUACUGGUAUAAUCAGUUUUGUUUAUAAAAUUAAACUAAAG | |
| | GAAAAAUGAUGAUUAACUAGGACAUAAUGGGUCAUCUUUUUAGGUAGC | |
| | CAUUGUUGUGAGAAAUACAAUAUAGAAUUAUAUGCUAGUUCCUAAGGU | |
| | UUAUUACCUCACCCAAUGCUGAAUUAAGCUACAAGUUUAUAACAAGUA | |
| | GAAAGAACCAUCGAUGUGGUUUUAAUAGAUCCAAGGCACUCAUAUUUU | |
| | AAAACCAAAUGAUAGAAUAAACUUGUUCUGUUUUUCUGUUAAUUUGUC | |
| | AAUUCAAGGCCUUUUUUCUUCCUUUCCAAUUGAUACAUUUAACCCUUU | |
| | AGAGACAGACAUUUAGCUCAUAGAGAUUUUUUUUCAGUGCUAUCUAUU | |
| | CUGUCUAUAGAGGGUUAAUCCAAAGACUGUUUUUCCUCCUCACGUUAU | |
| | AAAAUAAAACUGUACAUGAUAUGUAUUACAGAAUGUAUGCAGCAUGGU | |
| | CUUUUUCUCUCUCUCUCUCUUUUUCUCUCAGAACGGAACUGGAAACAG | |
| | CAACAUGUUUGCUCAGCAACGAAUUAGGGACAAUUUAAAAUAGCCAUA | |
| | ACAUACCAUACAUGCUGUCUAAGUUUAAAAAAAAACAUACACAACAUG | |
| | UAAAUUAUUGCACAAGAGAAAGGCUCAAAGUUUGCGUAAAAUGCAAUA | |
| | GUAUUGCCCCAUACAGAUCAUGCAUUCAAACGGUGAGAACAUAAAGGA | |
| | AAAAAAAAAAAAAGGAAAAAGAAAAAAGAAAAAGAAAAAGAAAAAAAA | |
| | CAGGUGUGCUGGUGACAAGCACUCUCAUAUUCUUAGCUUCGUUACUUC | |
| | UGUUUGUUUGUUUGUUUGUUUAAAUCACAUGGGACUAGAAAAAAAUCC | |
| | UACAGGGAGUGGGGCUGGAGGGCGAUGGGGAAGGGGAGUGGUGAAAAA | |
| | GGGGGUGUCAGGUGGGAGUGAGGGAGGGGUAUUAAUAUACCUCUAUUC | |
| | AGUUUUUAUAUCAUUAUUCAACACUCGAUCACUGUGCCAUUUUUUCAU | |
| | GUGUUUCUCCAGGGUACUGUACACGCUAAAAGGCAUCUUACAAAUUUC | |
| | ACAUUUGUAAACGUCCUUCCCCACCUGGCCAUGCGUUUUCAUGUGCCU | |
| | GGUGAGCUUGCUACUCUGGGCACAGGCAUAGUUGCACAGCUCGCAUUU | |
| | AUAAGGCCUUUCGCCCGUGUGGCUUCUCCUGUGGACAGUGAGAUUGCU | |
| | ACAGUUCUUGAAGACUUUCCCACAGUACUCACAAGUGUCGCUGCGUCU | |
| | GCCCUCUUUUGAGCUGGGCCUGCCCGGGCCCGGACCACUAAUAUGGGG | |
| | CGUGCUCCCUCCACUUCCCGUGCCGCUGCGCCCCGAGAUCCCUCCGUC | |
| | CAGCUCCCCGGGCGGUGUGGAGAAGCGCAAACUCCCGUUCUCCGAGGA | |
| | GUGCUCCGACGAGGAGGCAAAAGGCGAUUGUCUGGAGUCUCCGAAGCU | |
| | AAGGAAGGGAUCUUUGAGCUGCCUGGAGGCCGCGUAGCCGGCGAGCCA | |
| | CUGCGAGUACACGUUCUCCGUGUUGGGCAUCGCGGCCGGGGGCAGGUC | |
| | GAACUCCUUCUCGAGCUUGAUGCGCUUAGAGAAGGGGCUCAGCGAGCU | |
| | GGGGCUGCCCAGCAGCAGCUUUUUGGACAGGCCCCCCGAGGCCGACUC | |
| | GCCCGGGGAGCAGCCGCGGCCAUUAACAGUGCCAUCGUCUAUGCGGUC | |
| | CGACUCGCCGGCCACCGAGUCUUCGUCGCAAGUGUCCCUGUGGCCCUC | |
| | GGCCUCGGCCAGGUGGCCGCGCUUAUGCUUCUCGCCCAGGACCUGGUG | |
| | GAAGGCCUCGCUGAAGUGCUGCAUGGAGCUGAGCACCAUGCCCUGCAU | |
| | GACGUCGGGCAGGGCGCGGCUCUCGUCGCCCACGCCCACGACCGCGCC | |
| | CCGCGAGCUGUUCUCGUGGUGGCGCGCCGCCUCCAGGCUCAGCCCGAA | |
| | GCCGUAGUCCACCCUCUCGCUCUCCGUCAGCUCCUCCUCCUCCUCUUC | |
| | CUCCUCUUCUUCCUCUUCCUCGUCGUCCUCCUCUUCCUCCUCGUCCCC | |
| | GUUCUCCGGGAUCAGGUUGGGGUCGUUCUCGCUCUUGAACUUGGCCAC | |
| | CACGGACUUGAGCGCGCUGCUGGCGCUGCCCACCAAGUCGCUGGUGCC | |
| | GGGUUCCGGGGAGCUGGCGGUGGAGAGACCGUCGUCGGACUUGACCGU | |
| | CAUGGGGGACGAUUUGUGCAUGUGCGUCUUCAUGUGGCGCUUCAGCUU | |
| | GCUGGCCUGGGUGCACGCGUGGUCGCACAGGUUGCACUUGUAGGGCUU | |
| | CUCGCCCGUGUGGCUGCGCCGGUGCACCACCAGGUUGCUCUGAAAUUU | |
| | GAACGUCUUGCCGCAGAACUCGCAUGACUUGGACUUGACCGGGGGCUG | |
| | GGAGGGAGGAGGGGCGGAUUGCAGAGGAGGGAGGGGGGGCGUCGCCAG | |
| | GAAGGGCGGCUUGCUACCUGGCUGGAAUGGUUGCAGUAACCUUUGCAU | |
| | AGGGCUGGGCCGGCCUGGGGACAGCGGUGGGCUAGACGUGUUCCCUGC | |
| | CAGCUCUCUAAGUCUCCUAGAGAAAUCCAUGGCGGGAGGCUCCAUAGC | |
| | CAUUGGAUUCAACCGCAGCACCCUGUCAAAGGCACUCGGGUGAUGGGU | |
| | GGCCAGGGCCAUCUCUUCCGCCCCCAGGCGCUCUAUGCGGUGGGGGUC | |
| | CAAGUGAUGUCUCGGUGGUGGACUAAACAGGGGGGGAGUGGGUGGAAA | |
| | GCGCCCUUCUGCCAGGCCGGAAGCCUCUCUCGAUACUGAUCCUGGUAU | |
| | UCUUAGCAGGUUAAAGGGGUUAUUGUCUGCAAUAUGAAUCCCAUGGAG | |
| | AGGUGGCUGGGAAGGACAUUCUGCACCUAGUCCUGAAGGGAUACCAAC | |
| | CCGCGGGGUCAGGGGACUUCCGUGUUCGCUUUCUAAGUAGAUUCUUAA | |
| | UCCAUGAGUGUUCUGUGCGUGUUGCAAGAGAAACCAUGCACUGGUGAA | |
| | UGGCUGUUUGCAAGUUGUACAUGUGUAGCUGCUGGGCUCAUCUUUACA | |
| | AAUACCCUGCGGGGCAUAUUCUGCACUCAUCCCAGGCGUGGGGAUUAG | |
| | AGCUCCAUGUGCAGAACGAGGGGAGGAGAGGCCCCUCCAGUGCAGAAG | |
| | UUUAUCUGCUAUGUGUUCCUGUUUGGGGCAAAUUCCUCUAGAUGACGU | |
| | UGAUAAACAAUCGUCAUCCUCUGGCGUGACCUGGAUGCCAACCUCCAC | |
| | GGGAUUGGAUGCUUUUUUCAUCUCGAUUGGUGAAGGGGAAGGUGGCUU | |
| | AUCCACAGCUUUUUCUAAGCAGAGGCUGCCAUUGCAUUGUUUCCGUUU | |
| | GUGCUCGAUAAAAAUAAGAAUGUCCCCCAAUGGGAAGUUCAUCUGGCA | |
| | CUGCCCACAGGUGAGGAGGUCAUGAUCCCCUUCUGGAGCUCCCAACGG | |
| | GCCGUGGUCUGGUUCAUCAUCUGUAAGAAUGGCUUCAAGAGGCUCGGG | |
| | CGAGAAUUCCCGUUUGCUUAAGUGCUGGGGUUUGCCUUGCUUGCGGCG | |
| | AGACAUGGUGGGCUGCGGGGGGGGCGGCGGCGGCGGCGGCGGCGGCGG | |
| | CGGGCGGACGACGGCUCGGUUCACAUCGGGAGAGCCGGGUUAGAAAGA | |
| | AGGAGACUCCAGAGAAAAUAUCUUCAUCAGUGCCUUUUGACAUCCAAA | |
| | AUAAAUUAGAAAUAAUACAAAGAUGGCGCAGGGAAGAUGAAUUGUGGG | |
| | AGAGCCGUCAUGGCUUUUUUUUAAGCAAAAAAAAAAAAAAAAAAAAAA | |
| | AAAAAAAGAGGGAGAGAGAGAGAAGAGAGAUAGAGGGAGAGAGAGAGA | |
| | GAGAGAUGAAAAAAAUGGCAAAAGCCCCCCUGAGCUGCAAGUUCAAGU | |
| | GCGGACGUGACGUCCCUGCGAACUUGAACGUCAGGAGUCUGGAUGGAC | |
| | AGAGAC | |
| hBCL11A | MSRRKQGKPQHLSKREFSPEPLEAILTDDEPDHGPLGAPEGDHDLLTC | 3 |
| Isoform 1 | GQCQMNFPLGDILIFIEHKRKQCNGSLCLEKAVDKPPSPSPIEMKKAS | |
| Protein | NPVEVGIQVTPEDDDCLSTSSRGICPKQEHIADKLLHWRGLSSPRSAH | |
| NCBI Ref.: | GALIPTPGMSAEYAPQGICKDEPSSYTCTTCKOPFTSAWFLLQHAQNT | |
| NP_075044.2 | HGLRIYLESEHGSPLTPRVGIPSGLGAECPSQPPLHGIHIADNNPFNL | |
| | LRIPGSVSREASGLAEGRFPPTPPLFSPPPRHHLDPHRIERLGAEEMA | |
| | LATHHPSAFDRVLRLNPMAMEPPAMDFSRRLRELAGNTSSPPLSPGRP | |
| | SPMQRLLQPFQPGSKPPFLATPPLPPLQSAPPPSQPPVKSKSCEFCGK | |
| | IFKFQSNLVVHRRSHTGEKPYKCNLCDHACTQASKLKRHMKTHMHKSS | |
| | PMTVKSDDGLSTASSPEPGTSDLVGSASSALKSVVAKFKSENDPNLIP | |
| | ENGDEEEEEDDEEEEEEEEEEEEELTESERVDYGFGLSLEAARHHENS | |
| | SRGAVVGVGDESRALPDVMQGMVLSSMQHFSEAFHQVLGEKHKRGHLA | |
| | EAEGHRDTCDEDSVAGESDRIDDGTVNGRGCSPGESASGGLSKKLLLG | |
| | SPSSLSPFSKRIKLEKEFDLPPAAMPNTENVYSQWLAGYAASROLKDP | |
| | FLSFGDSRQSPFASSSEHSSENGSLRFSTPPGELDGGISGRSGTGSGG | |
| | STPHISGPGPGRPSSKEGRRSDTCEYCGKVFKNCSNLTVHRRSHTGER | |
| | PYKCELCNYACAQSSKLTRHMKTHGQVGKDVYKCEICKMPFSVYSTLE | |
| | KHMKKWHSDRVLNNDIKTE | |

In some embodiments, the agent (e.g., RNAi agent, dsRNA agent) comprises one or more RNA molecule. In some embodiments, the agent (e.g., RNAi agent, dsRNA agent) comprises an antisense strand. In some embodiments, the agent (e.g., RNAi agent, dsRNA agent) comprises a sense strand. In some embodiments, the agent comprises one or more single stranded RNA (ssRNA) molecules. In some embodiments, the agent (e.g., RNAi agent, dsRNA agent) comprises a dsRNA agent.

In some embodiments, the agent (e.g., RNAi agent) comprises a dsRNA agent comprising a sense strand and an antisense strand. In some embodiments, the agent (e.g., RNAi agent) comprises a dsRNA agent comprising a sense strand and an antisense strand that form a double stranded region. In some embodiments, the agent (e.g., RNAi agent) comprises a dsRNA agent comprising a sense strand and an antisense strand that hybridize to form a double stranded region. In some embodiments, the sense strand and the antisense strand are part of a single nucleic acid molecule (e.g., a single nucleic acid molecule comprising a hairpin loop). In some embodiments, the sense strand and the antisense strand are separate nucleic acid molecules.

### 4.2.1 Antisense Strand

### 4.2.1.1 Targeting Region

As described above, antisense strands (e.g., described herein) comprise a region of complementarity that comprises a nucleotide sequence that is at least partially (e.g., substantially, fully) complementary to the nucleotide sequence of a target nucleic acid molecule (e.g., a target mRNA (e.g., a BCL11A mRNA), a portion of a target mRNA (e.g., a BCL11A mRNA)). In some embodiments, the nucleotide sequence of the region of complementarity is at least substantially complementary to the nucleotide sequence of the target nucleic acid molecule (e.g., a target mRNA (e.g., a BCL11A mRNA), a portion of a target mRNA (e.g., a BCL11A mRNA)). In some embodiments, the nucleotide sequence of the region of complementarity is fully complementary to the nucleotide sequence of the target nucleic acid molecule (e.g., a target mRNA (e.g., a BCL11A mRNA), a portion of a target mRNA (e.g., a BCL11A mRNA)).

In some embodiments, the nucleotide sequence of the region of complementarity is at least 70%, 75%, 80%, 85%, 90%, 91%, 92%, 93%, 94%, 95%, 96%, 97%, 98%, 99%, or 100% complementary to the nucleotide sequence of the target nucleic acid molecule (e.g., a target mRNA (e.g., a BCL11A mRNA), a portion of a target mRNA (e.g., a BCL11A mRNA)). For example, the nucleotide sequence of the region of complementarity may be at least 70% complementary to the nucleotide sequence of the target nucleic acid molecule (e.g., a target mRNA (e.g., a BCL11A mRNA), a portion of a target mRNA (e.g., a BCL11A mRNA)). The nucleotide sequence of the region of complementarity may be at least 75% complementary to the nucleotide sequence of the target nucleic acid molecule (e.g., a target mRNA (e.g., a BCL11A mRNA), a portion of a target mRNA (e.g., a BCL11A mRNA)). The nucleotide sequence of the region of complementarity may be at least 80% complementary to the nucleotide sequence of the target nucleic acid molecule (e.g., a target mRNA (e.g., a BCL11A mRNA), a portion of a target mRNA (e.g., a BCL11A mRNA)). The nucleotide sequence of the region of complementarity may be at least 85% complementary to the nucleotide sequence of the target nucleic acid molecule (e.g., a target mRNA (e.g., a BCL11A mRNA), a portion of a target mRNA (e.g., a BCL11A mRNA)). The nucleotide sequence of the region of complementarity may be at least 90% complementary to the nucleotide sequence of the target nucleic acid molecule (e.g., a target mRNA (e.g., a BCL11A mRNA), a portion of a target mRNA (e.g., a BCL11A mRNA)). The nucleotide sequence of the region of complementarity may be at least 95% complementary to the nucleotide sequence of the target nucleic acid molecule (e.g., a target mRNA (e.g., a BCL11A mRNA), a portion of a target mRNA (e.g., a BCL11A mRNA)). In some embodiments, the nucleotide sequence of the region of complementarity is at least 80%, 85%, 90%, 91%, 92%, 93%, 94%, 95%, 96%, 97%, 98%, 99%, or 100% complementary to the nucleotide sequence of the target nucleic acid molecule (e.g., a target mRNA (e.g., a BCL11A mRNA), a portion of a target mRNA (e.g., a BCL11A mRNA)). In some embodiments, the nucleotide sequence of the region of complementarity is at least 95%, 96%, 97%, 98%, 99%, or 100% (e.g., in some embodiments, preferably at least 95%, more preferably at least 98%) complementary to the nucleotide sequence of the target nucleic acid molecule (e.g., a target mRNA (e.g., a BCL11A mRNA), a portion of a target mRNA (e.g., a BCL11A mRNA)). In some embodiments, the nucleotide sequence of the region of complementarity is 100% complementary to the nucleotide sequence of the target nucleic acid molecule (e.g., a target mRNA (e.g., a BCL11A mRNA), a portion of a target mRNA (e.g., a BCL11A mRNA)).

In some embodiments, the nucleotide sequence of the region of complementarity comprises or consists of one or more non-complementary nucleotide mismatches relative to the nucleotide sequence of the target nucleic acid molecule (e.g., a target mRNA (e.g., a BCL11A mRNA), a portion of a target mRNA (e.g., a BCL11A mRNA)). In some embodiments, the nucleotide sequence of the region of complementarity comprises or consists of no more than 5 (e.g., 4, 3, 2, 1, or 0) non-complementary nucleotide mismatches relative to the nucleotide sequence of the target nucleic acid molecule. In some embodiments, the nucleotide sequence of the region of complementarity comprises or consists of no more than 3 (e.g., 2, 1, or 0) non-complementary nucleotide mismatches relative to the nucleotide sequence of the target nucleic acid molecule. In some embodiments, the nucleotide sequence of the region of complementarity comprises or consists of no more than 2 (e.g., 1 or 0) non-complementary nucleotide mismatches relative to the nucleotide sequence of the target nucleic acid molecule. In some embodiments, the nucleotide sequence of the region of complementarity comprises or consists of no more than 1 (e.g., 0) non-complementary nucleotide mismatch relative to the nucleotide sequence of the target nucleic acid molecule. In some embodiments, the nucleotide sequence of the region of complementarity comprises 0 non-complementary nucleotide mismatches relative to the nucleotide sequence of the target nucleic acid molecule. In some embodiments, the region of complementarity comprises one or more (e.g., 2, 3, or more) non-complementary nucleotide mismatches relative to the nucleotide sequence of the target nucleic acid molecule, wherein the one or more non-complementary nucleotide mismatches are within the last 5 (e.g., 4, 3, 2, or 1) nucleotides from either the 5′- and/or 3′-end of the region of complementarity. In some embodiments, the region of complementarity comprises at least one but not more than 3 non-complementary nucleotide mismatches relative to the nucleotide sequence of the target nucleic acid molecule, wherein the one or more non-complementary nucleotide mismatches are within the last 5 (e.g., 4, 3, 2, or 1) nucleotides from either the 5′- and/or 3′-end of the region of complementarity. In some embodiments, the region of complementarity comprises one or more (e.g., 2, 3, or more) non-complementary nucleotide mismatches relative to the nucleotide sequence of the target nucleic acid molecule, wherein the one or more non-complementary nucleotide mismatches are within the last 3 (e.g., 2 or 1) nucleotides from either the 5′- and/or 3′-end of the region of complementarity. In some embodiments, the region of complementarity comprises at least one but not more than 3 non-complementary nucleotide mismatches relative to the nucleotide sequence of the target nucleic acid molecule, wherein the one or more non-complementary nucleotide mismatches are within the last 3 (e.g., 2 or 1) nucleotides from either the 5′- and/or 3′-end of the region of complementarity. Methods known in the art and described herein can be utilized to evaluate the effect of any non-complementary mismatches between an antisense strand and a target nucleic acid molecule on functional properties (e.g., inhibition of expression of the target nucleic acid molecule (e.g., a target mRNA (e.g., a BCL11A mRNA), a portion of a target mRNA (e.g., a BCL11A mRNA))).

In some embodiments, the region of complementarity comprises or consists of from about 15-30 nucleotides, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides. In some embodiments, the region of complementarity comprises from about 18-25, 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 nucleotides. In some embodiments, the region of complementarity comprises from about 19-21 (e.g., 19-20) nucleotides. In some embodiments, the region of complementarity comprises or consists of about 15, 16, 17, 18, 19, 20, 21, 22, 23, 24, 25, 26, 27, 28, 29, or 30 nucleotides. In some embodiments, the region of complementarity comprises or consists of about 19, 20, 21, 22, or 23 nucleotides. In some embodiments, the region of complementarity comprises or consists of about 19 nucleotides. In some embodiments, the region of complementarity comprises or consists of about 20 nucleotides. In some embodiments, the region of complementarity comprises or consists of about 21 nucleotides. In some embodiments, the region of complementarity comprises or consists of about 22 nucleotides. In some embodiments, the region of complementarity comprises or consists of about 23 nucleotides. Ranges and lengths intermediate to the above recited ranges and lengths are also contemplated to be part of the disclosure.

In some embodiments, the target nucleic acid molecule is part (e.g., a contiguous portion) of a larger nucleic acid molecule. For example, in some embodiments, the target nucleic acid molecule is a portion (e.g., a contiguous portion) of a target mRNA (e.g., a BCL11A mRNA). In some embodiments, the target nucleic acid molecule is a contiguous nucleotide sequence of a target mRNA (e.g., a BCL11A mRNA) of sufficient length to allow it to be a substrate for cleavage directed by an RNAi agent (e.g., an RNAi agent described herein, e.g., a dsRNA agent (e.g., described herein)) (i.e., cleavage through a RISC pathway).

In some embodiments, the target nucleic acid molecule is a target mRNA (e.g., a BCL11A mRNA). In some embodiments, the target nucleic acid molecule is at least a portion (e.g., a portion) of a target mRNA (e.g., a BCL11A mRNA). In some embodiments, the target nucleic acid molecule is at least a portion (e.g., a portion) of an mRNA (e.g., a BCL11A mRNA) formed in the expression of a target gene (e.g., a mammalian, primate, human, non-human primate, mouse, and/or rat gene) (e.g., a BCL11A gene). In some embodiments, the target nucleic acid molecule is at least a portion (e.g., a portion) of a BCL11A (e.g., hBCL11A) mRNA. In some embodiments, the target nucleic acid molecule is at least a portion (e.g., a portion) of an mRNA formed in the expression of a BCL11A (e.g., hBCL11A) gene. In some embodiments, the target nucleic acid molecule comprises at least a portion (e.g., a portion) of the nucleotide sequence set forth in SEQ ID NO: 1 (or a variant or fragment thereof). In some embodiments, the target nucleic acid molecule comprises at least a portion (e.g., a portion) of an mRNA encoding a target protein. In some embodiments, the target nucleic acid molecule comprises at least a portion (e.g., a portion) of an mRNA encoding a BCL11A (e.g., hBCL11A) protein. In some embodiments, the target nucleic acid molecule comprises at least a portion (e.g., a portion) of an mRNA sequence encoding a protein comprising the amino acid sequence set forth in SEQ ID NO: 3 (or a variant or fragment thereof).

In some embodiments, the target nucleic acid molecule comprises or consists of from about 19-30 nucleotides, e.g., 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, 21-22, 22-30, 22-29, 22-28, 22-27, 22-26, 22-25, 22-24, 22-23, 23-30, 23-29, 23-28, 23-27, 23-26, 23-27, 23-26, 23-25, or 23-24 nucleotides. In some embodiments, the target nucleic acid molecule comprises or consists of from about 19-25 nucleotides. In some embodiments, the target nucleic acid molecule comprises or consists of from about 19-23 nucleotides. In some embodiments, the target nucleic acid molecule comprises or consists of from about 21-25 nucleotides. In some embodiments, the target nucleic acid molecule comprises or consists of from about 21-23 nucleotides. In some embodiments, the target nucleic acid molecule comprises or consists of about 19, 18, 20, 21, 22, 23, 24, 25, 26, 27, 28, 29, or 30 nucleotides. In some embodiments, the target nucleic acid molecule comprises or consists of about 19 nucleotides. In some embodiments, the target nucleic acid molecule comprises or consists of about 20 nucleotides. In some embodiments, the target nucleic acid molecule comprises or consists of about 21 nucleotides. In some embodiments, the target nucleic acid molecule comprises or consists of about 23 nucleotides. Ranges and lengths intermediate to the above recited ranges and lengths are also contemplated to be part of the disclosure.

### 4.2.1.2 Overall Length

In some embodiments, the antisense strand comprises or consists of from about 15-30 nucleotides (e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides). In some embodiments, the antisense strand comprises or consists of from about 18-25 nucleotides (e.g., 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 nucleotides). In some embodiments, the antisense strand comprises or consists of from about 19-25 nucleotide (e.g., 19-20, 19-21, 19-22, 19-23, 19-24, 19-25, 20-21, 20-22, 20-23, 20-24, 20-25, 21-22, 21-23, 21-24, 21-25, 22-23, 22-24, 22-25, 23-24, 23-25, 24-25 nucleotides). In some embodiments, the antisense strand comprises or consists of from about 15-30, 16-30, 17-30, 18-30, 19-30 20-30, 21-30, 22-30, 23-30, 24-30, 25-30, 36-30, 27-30, 28-30-, 29-30, 19-20, 19-21, 19-22, 19-23, 19-24, or 19-25 nucleotides.

In some embodiments, the antisense strand comprises or consists of not more than about 15, 16, 17, 18, 19, 20, 21, 22, 23, 24, 25, 26, 27, 28, 29, or 30 nucleotides. In some embodiments, the antisense strand comprises or consists of about 15, 16, 17, 18, 19, 20, 21, 22, 23, 24, 25, 26, 27, 28, 29, or 30 nucleotides. In some embodiments, the antisense strand comprises or consists of about 21 nucleotides. In some embodiments, the antisense strand comprises or consists of about 23 nucleotides. In preferred embodiments, the antisense strand comprises or consists of about 23 nucleotides. In preferred embodiments, the antisense strand comprises 23 nucleotides. In preferred embodiments, the antisense strand consists of 23 nucleotides. Ranges and lengths intermediate to the above recited ranges and lengths are also contemplated to be part of the disclosure.

### 4.2.1.3 Exemplary Antisense Strands

In some embodiments, the nucleotide sequence of the antisense strand differs by no more than 5 (e.g., 0, 1, 2, 3, 4, or 5) nucleotides from the nucleotide sequence of any one of the antisense strands set forth in any one of Tables 2-5 (e.g., Table 3 or 5). In some embodiments, the nucleotide sequence of the antisense strand differs by no more than 3 (e.g., 0, 1, 2, or 3) nucleotides from the nucleotide sequence of any one of the antisense strands set forth in any one of Tables 2-5 (e.g., Table 3 or 5). In some embodiments, the nucleotide sequence of the antisense strand differs by no more than 2 (e.g., 0, 1, or 2) nucleotides from the nucleotide sequence of any one of the antisense strands set forth in any one of Tables 2-5 (e.g., Table 3 or 5). In some embodiments, the nucleotide sequence of the antisense strand differs by no more than 1 (e.g., 0 or 1) nucleotide from the nucleotide sequence of any one of the antisense strands set forth in any one of Tables 2-5 (e.g., Table 3 or 5).

In some embodiments, the nucleotide sequence of the antisense strand comprises the nucleotide sequence of any one of the antisense strands set forth in any one of Tables 2-5 (e.g., Table 3 or 5) differing by no more than 5 (e.g., 0, 1, 2, 3, 4, or 5) nucleotides from the antisense strand set forth in any one of Tables 2-5 (e.g., Table 3 or 5). In some embodiments, the nucleotide sequence of the antisense strand comprises the nucleotide sequence of any one of the antisense strands set forth in any one of Tables 2-5 (e.g., Table 3 or 5) differing by no more than 3 (e.g., 0, 1, 2, or 3) nucleotides from the antisense strand set forth in any one of Tables 2-5 (e.g., Table 3 or 5). In some embodiments, the nucleotide sequence of the antisense strand comprises the nucleotide sequence of any one of the antisense strands set forth in any one of Tables 2-5 (e.g., Table 3 or 5) differing by no more than 2 (e.g., 0, 1, or 2) nucleotides from the antisense strand set forth in any one of Tables 2-5 (e.g., Table 3 or 5). In some embodiments, the nucleotide sequence of the antisense strand comprises the nucleotide sequence of any one of the antisense strands set forth in any one of Tables 2-5 (e.g., Table 3 or 5) differing by no more than 1 (e.g., 0 or 1) nucleotide from the antisense strand set forth in any one of Tables 2-5 (e.g., Table 3 or 5).

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence of any one of the antisense strands set forth in any one of Tables 2-5 (e.g., Table 3 or 5). In some embodiments, the nucleotide sequence of the antisense strand is at least 90%, 91%, 92%, 93%, 94%, 95%, 96%, 97%, 98%, 99% or 100% identical to the nucleotide sequence of any one of the antisense strands set forth in any one of Tables 2-5 (e.g., Table 3 or 5). In some embodiments, the nucleotide sequence of the antisense strand is at least 95%, 96%, 97%, 98%, 99% or 100% identical to the nucleotide sequence of any one of the antisense strands set forth in any one of Tables 2-5 (e.g., Table 3 or 5). In some embodiments, the nucleotide sequence of the antisense strand is at least 96%, 97%, 98%, 99% or 100% identical to the nucleotide sequence of any one of the antisense strands set forth in any one of Tables 2-5 (e.g., Table 3 or 5). In some embodiments, the nucleotide sequence of the antisense strand is at least 97%, identical to the nucleotide sequence of any one of the antisense strands set forth in any one of Tables 2-5 (e.g., Table 3 or 5). In some embodiments, the nucleotide sequence of the antisense strand is at least 98% identical to the nucleotide sequence of any one of the antisense strands set forth in any one of Tables 2-5 (e.g., Table 3 or 5). In some embodiments, the nucleotide sequence of the antisense strand is at least 99% identical to the nucleotide sequence of any one of the antisense strands set forth in any one of Tables 2-5 (e.g., Table 3 or 5). In some embodiments, the nucleotide sequence of the antisense strand is 100% identical to the nucleotide sequence of any one of the antisense strands set forth in any one of Tables 2-5 (e.g., Table 3 or 5).

In some embodiments, the nucleotide sequence of the antisense strand comprises the nucleotide sequence of any one of the antisense strands set forth in any one of Tables 2-5 (e.g., Table 3 or 5). In some embodiments, the nucleotide sequence of the antisense strand consists of the nucleotide sequence of any one of the antisense strands set forth in any one of Tables 2-5 (e.g., Table 3 or 5).

Tables 2-5 (e.g., Table 3 or 5) further identify the target nucleic acid molecule within the cited reference BCL11A mRNA transcript (SEQ ID NO: 1) targeted by each of the antisense strands. As such, the disclosure further provides antisense strands wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence of any one of the antisense strands set forth in any one of Tables 2-5 (e.g., Table 3 or 5) and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the BCL11A mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5). In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5). In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5). In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5).

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5). In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5). In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5).

In some embodiments, the nucleotide sequence of the antisense strand comprises the nucleotide sequence of any one of the antisense strands set forth in any one of Tables 2-5 (e.g., Table 3 or 5) differing by no more than 3 (e.g., 0, 1, 2, or 3) nucleotides from the antisense strand set forth in any one of Tables 2-5 (e.g., Table 3 or 5). In some embodiments, the nucleotide sequence of the antisense strand comprises the nucleotide sequence of any one of the antisense strands set forth in any one of Tables 2-5 (e.g., Table 3 or 5).

Tables 2-5 further identify the target nucleic acid molecule within the cited reference BCL11A mRNA transcript (SEQ ID NO: 1) targeted by each of the antisense strands. As such, the disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5) and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the BCL11A mRNA transcript targeted by the select antisense strand.

In some embodiments, the nucleotide sequence of the antisense strand differs by no more than 5 (e.g., 0, 1, 2, 3, 4, or 5) nucleotides from the nucleotide sequence set forth in any one of SEQ ID NOS: 4-1576 (e.g., any one of SEQ ID NOS: 4-19 or 772-787). In some embodiments, the nucleotide sequence of the antisense strand differs by no more than 3 (e.g., 0, 1, 2, or 3) nucleotides from the nucleotide sequence set forth in any one of SEQ ID NOS: 4-1576 (e.g., any one of SEQ ID NOS: 4-19 or 772-787). In some embodiments, the nucleotide sequence of the antisense strand differs by no more than 2 (e.g., 0, 1, or 2) nucleotides from the nucleotide sequence set forth in any one of SEQ ID NOS: 4-1576 (e.g., any one of SEQ ID NOS: 4-19 or 772-787). In some embodiments, the nucleotide sequence of the antisense strand differs by no more than 1 (e.g., 0 or 1) nucleotide from the nucleotide sequence set forth in any one of SEQ ID NOS: 4-1576 (e.g., any one of SEQ ID NOS: 4-19 or 772-787).

In some embodiments, the nucleotide sequence of the antisense strand comprises the nucleotide sequence set forth in any one of SEQ ID NOS: 4-1576 (e.g., any one of SEQ ID NOS: 4-19 or 772-787) differing by no more than 5 (e.g., 0, 1, 2, 3, 4, or 5) nucleotides from the nucleotide sequence set forth in any one of SEQ ID NOS: 4-1576 (e.g., any one of SEQ ID NOS: 4-19 or 772-787). In some embodiments, the nucleotide sequence of the antisense strand comprises the nucleotide sequence set forth in any one of SEQ ID NOS: 4-1576 (e.g., any one of SEQ ID NOS: 4-19 or 772-787) differing by no more than 3 (e.g., 0, 1, 2, or 3) nucleotides from the nucleotide sequence set forth in any one of SEQ ID NOS: 4-1576 (e.g., any one of SEQ ID NOS: 4-19 or 772-787). In some embodiments, the nucleotide sequence of the antisense strand comprises the nucleotide sequence set forth in any one of SEQ ID NOS: 4-1576 (e.g., any one of SEQ ID NOS: 4-19 or 772-787) differing by no more than 2 (e.g., 0, 1, or 2) nucleotides from the nucleotide sequence set forth in any one of SEQ ID NOS: 4-1576 (e.g., any one of SEQ ID NOS: 4-19 or 772-787). In some embodiments, the nucleotide sequence of the antisense strand comprises the nucleotide sequence set forth in any one of SEQ ID NOS: 4-1576 (e.g., any one of SEQ ID NOS: 4-19 or 772-787) differing by no more than 1 (e.g., 0 or 1) nucleotide from the nucleotide sequence set forth in any one of SEQ ID NOS: 4-1576 (e.g., any one of SEQ ID NOS: 4-19 or 772-787).

In some embodiments, the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in any one of SEQ ID NOS: 4-1576 (e.g., any one of SEQ ID NOS: 4-19 or 772-787). In some embodiments, the nucleotide sequence of the antisense strand is at least 90%, 91%, 92%, 93%, 94%, 95%, 96%, 97%, 98%, 99% or 100% identical to the nucleotide sequence set forth in any one of SEQ ID NOS: 4-1576 (e.g., any one of SEQ ID NOS: 4-19 or 772-787). In some embodiments, the nucleotide sequence of the antisense strand is at least 95%, 96%, 97%, 98%, 99% or 100% identical to the nucleotide sequence set forth in any one of SEQ ID NOS: 4-1576 (e.g., any one of SEQ ID NOS: 4-19 or 772-787). In some embodiments, the nucleotide sequence of the antisense strand is at least 96%, 97%, 98%, 99% or 100% identical to the nucleotide sequence set forth in any one of SEQ ID NOS: 4-1576 (e.g., any one of SEQ ID NOS: 4-19 or 772-787). In some embodiments, the nucleotide sequence of the antisense strand is at least 97%, identical to the nucleotide sequence set forth in any one of SEQ ID NOS: 4-1576 (e.g., any one of SEQ ID NOS: 4-19 or 772-787). In some embodiments, the nucleotide sequence of the antisense strand is at least 98% identical to the nucleotide sequence set forth in any one of SEQ ID NOS: 4-1576 (e.g., any one of SEQ ID NOS: 4-19 or 772-787). In some embodiments, the nucleotide sequence of the antisense strand is at least 99% identical to the nucleotide sequence set forth in any one of SEQ ID NOS: 4-1576 (e.g., any one of SEQ ID NOS: 4-19 or 772-787). In some embodiments, the nucleotide sequence of the antisense strand is 100% identical to the nucleotide sequence set forth in any one of SEQ ID NOS: 4-1576 (e.g., any one of SEQ ID NOS: 4-19 or 772-787).

In some embodiments, the nucleotide sequence of the antisense strand comprises the nucleotide sequence set forth in any one of SEQ ID NOS: 4-1576 (e.g., any one of SEQ ID NOS: 4-19 or 772-787). In some embodiments, the nucleotide sequence of the antisense strand consists of the nucleotide sequence set forth in any one of SEQ ID NOS: 4-1576 (e.g., any one of SEQ ID NOS: 4-19 or 772-787).

Tables 2-5 further identify the target nucleic acid molecule within the cited reference BCL11A mRNA transcript (SEQ ID NO: 1) targeted by each of the antisense strands. As such, the disclosure further provides antisense strands wherein the nucleotide sequence of the antisense strand differs by no more than 5 (e.g., 0, 1, 2, 3, 4, or 5 (e.g., no more than 3 (e.g., 0, 1, 2, or 3))) nucleotides from the nucleotide sequence set forth in any one of SEQ ID NOS: 4-1576 (e.g., any one of SEQ ID NOS: 4-19 or 772-787) and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the BCL11A mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in any one of SEQ ID NOS: 4-1576 (e.g., any one of SEQ ID NOS: 4-19 or 772-787). In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in SEQ ID NOS: 4-1576. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in any one of SEQ ID NOS: 4-1576 (e.g., any one of SEQ ID NOS: 4-19 or 772-787). In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in any one of SEQ ID NOS: 4-1576 (e.g., any one of SEQ ID NOS: 4-19 or 772-787).

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in any one of SEQ ID NOS: 4-1576 (e.g., any one of SEQ ID NOS: 4-19 or 772-787). In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in any one of SEQ ID NOS: 4-1576 (e.g., any one of SEQ ID NOS: 4-19 or 772-787). In some embodiments, the antisense strand comprises from about 21-23 (e.g., 21-22, 22-23) contiguous nucleotides differing by no more than 3 (e.g., 0, 1, 2, or 3) nucleotides from the nucleotide sequence set forth in any one of SEQ ID NOS: 4-1576 (e.g., any one of SEQ ID NOS: 4-19 or 772-787).

In some embodiments, the nucleotide sequence of the antisense strand comprises the nucleotide sequence set forth in any one of SEQ ID NOS: 4-1576 (e.g., any one of SEQ ID NOS: 4-19 or 772-787) differing by no more than 3 (e.g., 0, 1, 2, or 3) nucleotides from the nucleotide sequence set forth in any one of SEQ ID NOS: 4-1576 (e.g., any one of SEQ ID NOS: 4-19 or 772-787). In some embodiments, the nucleotide sequence of the antisense strand comprises the nucleotide sequence set forth in any one of SEQ ID NOS: 4-1576 (e.g., any one of SEQ ID NOS: 4-19 or 772-787).

As described above, Tables 2-5 further identify the target nucleic acid molecule within the cited reference BCL11A mRNA transcript (SEQ ID NO: 1) targeted by each of the antisense strands. The disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in any one of SEQ ID NOS: 4-1576 (e.g., any one of SEQ ID NOS: 4-19 or 772-787) and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the BCL11A mRNA transcript targeted by the select antisense strand.

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands of any one of dsRNA agents 1-1590 (e.g., one of dsRNA agents 1-16 or 770-785) set forth in any one of Tables 2-5 (e.g., Table 3 or 5). In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands of any one of dsRNA agents 1-1590 (e.g., one of dsRNA agents 1-16 or 770-785) set forth in any one of Tables 2-5 (e.g., Table 3 or 5). In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands of any one of dsRNA agents 1-1590 (e.g., one of dsRNA agents 1-16 or 770-785) set forth in any one of Tables 2-5 (e.g., Table 3 or 5). In some embodiments, the antisense strand comprises at least 23 (e.g., 24, 25, 26, 27, 28, 29, 30) contiguous nucleotides differing by no more than 3 (e.g., 0, 1, 2, or 3) nucleotides from the nucleotide sequence of any one of the antisense strands of any one of dsRNA agents 1-1590 (e.g., one of dsRNA agents 1-16 or 770-785) set forth in any one of Tables 2-5 (e.g., Table 3 or 5).

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands of any one of dsRNA agents 1-1590 (e.g., one of dsRNA agents 1-16 or 770-785) set forth in any one of Tables 2-5 (e.g., Table 3 or 5). In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands of any one of dsRNA agents 1-1590 (e.g., one of dsRNA agents 1-16 or 770-785) set forth in any one of Tables 2-5 (e.g., Table 3 or 5). In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of any one of the antisense strands of any one of dsRNA agents 1-1590 (e.g., one of dsRNA agents 1-16 or 770-785) set forth in any one of Tables 2-5 (e.g., Table 3 or 5).

In some embodiments, the nucleotide sequence of the antisense strand comprises the nucleotide sequence of any one of the antisense strands of any one of dsRNA agents 1-1590 (e.g., one of dsRNA agents 1-16 or 770-785) set forth in any one of Tables 2-5 (e.g., Table 3 or 5) differing by no more than 3 (e.g., 0, 1, 2, or 3) nucleotides from the antisense strand set forth in any one of Tables 2-5 (e.g., Table 3 or 5). In some embodiments, the nucleotide sequence of the antisense strand comprises the nucleotide sequence of any one of the antisense strands of any one of dsRNA agents 1-1590 (e.g., one of dsRNA agents 1-16 or 770-785) set forth in any one of Tables 2-5 (e.g., Table 3 or 5).

As described above, Tables 2-5 further identify the target nucleic acid molecule within the cited reference BCL11A mRNA transcript (SEQ ID NO: 1) targeted by each of the antisense strands. The disclosure further provides antisense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands of any one of dsRNA agents 1-1590 (e.g., one of dsRNA agents 1-16 or 770-785) set forth in any one of Tables 2-5 (e.g., Table 3 or 5) and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the BCL11A mRNA transcript targeted by the select antisense strand.

It is to be understood, that although the antisense strands set forth in any one of Tables 2-5 (e.g., Table 3 or 5) are not described as being modified (e.g., comprising chemically modified nucleotides), conjugated, etc., the disclosure includes any antisense strand set forth in any one of Tables 2-5 (e.g., Table 3 or 5) that is unmodified, unconjugated, modified (e.g., as described herein), or conjugated (e.g., as described herein).

### 4.2.2 Sense Strand

### 4.2.2.1 Antisense Strand Complementarity

As described above, sense strands (e.g., described herein) comprise a region of complementarity that comprises a nucleotide sequence that is at least partially (e.g., substantially, fully) complementary to the nucleotide sequence of at least a portion of an antisense strand. As such, pairs of sense and antisense strands can hybridize to form a double stranded region (e.g., under conditions in which the pairs will be used).

In some embodiments, the nucleotide sequence of the region of complementarity is at least substantially complementary to the nucleotide sequence of at least a portion of an antisense strand. In some embodiments, the nucleotide sequence of the region of complementarity is fully complementary to the nucleotide sequence of at least a portion of an antisense strand.

In some embodiments, the nucleotide sequence of the region of complementarity is at least 70%, 75%, 80%, 85%, 90%, 91%, 92%, 93%, 94%, 95%, 96%, 97%, 98%, 99%, or 100% complementary to the nucleotide sequence of at least a portion of an antisense strand. For example, the nucleotide sequence of the region of complementarity may be at least 70% complementary to the nucleotide sequence of at least a portion of an antisense strand. The nucleotide sequence of the region of complementarity may be at least 75% complementary to the nucleotide sequence of at least a portion of an antisense strand. The nucleotide sequence of the region of complementarity may be at least 80% complementary to the nucleotide sequence of at least a portion of an antisense strand. The nucleotide sequence of the region of complementarity may be at least 85% complementary to the nucleotide sequence of at least a portion of an antisense strand. The nucleotide sequence of the region of complementarity may be at least 90% complementary to the nucleotide sequence of at least a portion of an antisense strand. The nucleotide sequence of the region of complementarity may be at least 95% complementary to the nucleotide sequence of at least a portion of an antisense strand. In some embodiments, the nucleotide sequence of the region of complementarity is at least 80%, 85%, 90%, 91%, 92%, 93%, 94%, 95%, 96%, 97%, 98%, 99%, or 100% complementary to the nucleotide sequence of at least a portion of an antisense strand. In some embodiments, the nucleotide sequence of the region of complementarity is at least 95%, 96%, 97%, 98%, 99%, or 100% complementary to the nucleotide sequence of at least a portion of an antisense strand.

In some embodiments, the nucleotide sequence of the region of complementarity comprises or consists of one or more non-complementary nucleotide mismatches relative to the nucleotide sequence of the at least a portion of an antisense strand. In some embodiments, the nucleotide sequence of the region of complementarity comprises or consists of no more than 5 (e.g., 4, 3, 2, 1, or 0) non-complementary nucleotide mismatches relative to the nucleotide sequence of the at least a portion of an antisense strand. In some embodiments, the nucleotide sequence of the region of complementarity comprises or consists of no more than 3 (e.g., 2, 1, or 0) non-complementary nucleotide mismatches relative to the nucleotide sequence of the at least a portion of an antisense strand. In some embodiments, the nucleotide sequence of the region of complementarity comprises or consists of no more than 2 (e.g., 1 or 0) non-complementary nucleotide mismatches relative to the nucleotide sequence of the at least a portion of an antisense strand. In some embodiments, the nucleotide sequence of the region of complementarity comprises or consists of no more than 1 (e.g., 0) non-complementary nucleotide mismatches relative to the nucleotide sequence of the at least a portion of an antisense strand. In some embodiments, the nucleotide sequence of the region of complementarity comprises 0 non-complementary nucleotide mismatches relative to the nucleotide sequence of the at least a portion of an antisense strand. In some embodiments, the region of complementarity comprises one or more (e.g., 2, 3, or more) non-complementary nucleotide mismatches relative to the nucleotide sequence of the at least a portion of an antisense strand, wherein the one or more non-complementary nucleotide mismatch is within the last 5 (e.g., 4, 3, 2, or 1) nucleotides from either the 5′- and/or 3′-end of the region of complementarity. In some embodiments, the region of complementarity comprises at least one but not more than 3 (e.g., 1, 2, or 3) non-complementary nucleotide mismatches relative to the nucleotide sequence of the at least a portion of an antisense strand, wherein the one or more non-complementary nucleotide mismatch is within the last 5 (e.g., 4, 3, 2, or 1) nucleotides from either the 5′- and/or 3′-end of the region of complementarity.

In some embodiments, the region of complementarity comprises from about 15-30 nucleotides, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides. In some embodiments, the region of complementarity comprises from about 18-25, 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 nucleotides. In some embodiments, the region of complementarity comprises from about 19-21 (e.g., 19-20) nucleotides. In some embodiments, the region of complementarity comprises or consists of about 15, 16, 17, 18, 19, 20, 21, 22, 23, 24, 25, 26, 27, 28, 29, or 30 nucleotides. In some embodiments, the region of complementarity comprises or consists of about 19, 20, or 21 nucleotides. In some embodiments, the region of complementarity comprises or consists of about 19 nucleotides. In some embodiments, the region of complementarity comprises or consists of about 20 nucleotides. In some embodiments, the region of complementarity comprises or consists of about 21 nucleotides. Ranges and lengths intermediate to the above recited ranges and lengths are also contemplated to be part of the disclosure.

### 4.2.2.2 Overall Length

In some embodiments, the sense strand comprises or consists of from about 15-30 nucleotides (e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides). In some embodiments, the sense strand comprises or consists of from about 18-25 nucleotides (e.g., 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 nucleotides). In some embodiments, the sense strand comprises or consists of from about 19-25 nucleotide (e.g., 19-20, 19-21, 19-22, 19-23, 19-24, 19-25, 20-21, 20-22, 20-23, 20-24, 20-25, 21-22, 21-23, 21-24, 21-25, 22-23, 22-24, 22-25, 23-24, 23-25, 24-25 nucleotides). In some embodiments, the sense strand comprises or consists of from about 15-30, 16-30, 17-30, 18-30, 19-30 20-30, 21-30, 22-30, 23-30, 24-30, 25-30, 36-30, 27-30, 28-30-, 29-30, 19-20, 19-21, 19-22, 19-23, 19-24, or 19-25 nucleotides.

In some embodiments, the sense strand comprises or consists of not more than about 15, 16, 17, 18, 19, 20, 21, 22, 23, 24, 25, 26, 27, 28, 29, or 30 nucleotides. In some embodiments, the sense strand comprises or consists of about 15, 16, 17, 18, 19, 20, 21, 22, 23, 24, 25, 26, 27, 28, 29, or 30 nucleotides. In some embodiments, the sense strand comprises or consists of about 19, 20, 21, 22, 23 nucleotides. In some embodiments, the sense strand comprises or consists of about 19, 20, 21 nucleotides. In some embodiments, the sense strand comprises or consists of about 20 nucleotides. In some embodiments, the sense strand comprises or consists of about 21 nucleotides. In some embodiments, the sense strand comprises or consists of about 21 nucleotides. In preferred embodiments, the sense strand comprises or consists of about 21 nucleotides. In preferred embodiments, the sense strand comprises 21 nucleotides. In preferred embodiments, the sense strand consists of 21 nucleotides. In some embodiments, the sense strand comprises or consists of about 23 nucleotides. Ranges and lengths intermediate to the above recited ranges and lengths are also contemplated to be part of the disclosure.

### 4.2.2.3 Exemplary Sense Strands

In some embodiments, the nucleotide sequence of the sense strand differs by no more than 5 (e.g., 0, 1, 2, 3, 4, or 5) nucleotides from the nucleotide sequence of any one of the sense strands set forth in any one of Tables 2-5 (e.g., Table 3 or 5). In some embodiments, the nucleotide sequence of the sense strand differs by no more than 3 (e.g., 0, 1, 2, or 3) nucleotides from the nucleotide sequence of any one of the sense strands set forth in any one of Tables 2-5 (e.g., Table 3 or 5). In some embodiments, the nucleotide sequence of the sense strand differs by no more than 2 (e.g., 0, 1, or 2) nucleotides from the nucleotide sequence of any one of the sense strands set forth in any one of Tables 2-5 (e.g., Table 3 or 5). In some embodiments, the nucleotide sequence of the sense strand differs by no more than 1 (e.g., 0 or 1) nucleotide from the nucleotide sequence of any one of the sense strands set forth in any one of Tables 2-5 (e.g., Table 3 or 5).

In some embodiments, the nucleotide sequence of the sense strand comprises the nucleotide sequence of any one of the sense strands set forth in any one of Tables 2-5 (e.g., Table 3 or 5) differing by no more than 5 (e.g., 0, 1, 2, 3, 4, or 5) nucleotides from the sense strand set forth in any one of Tables 2-5 (e.g., Table 3 or 5). In some embodiments, the nucleotide sequence of the sense strand comprises the nucleotide sequence of any one of the sense strands set forth in any one of Tables 2-5 (e.g., Table 3 or 5) differing by no more than 3 (e.g., 0, 1, 2, or 3) nucleotides from the sense strand set forth in any one of Tables 2-5 (e.g., Table 3 or 5). In some embodiments, the nucleotide sequence of the sense strand comprises the nucleotide sequence of any one of the sense strands set forth in any one of Tables 2-5 (e.g., Table 3 or 5) differing by no more than 2 (e.g., 0, 1, or 2) nucleotides from the sense strand set forth in any one of Tables 2-5 (e.g., Table 3 or 5). In some embodiments, the nucleotide sequence of the sense strand comprises the nucleotide sequence of any one of the sense strands set forth in any one of Tables 2-5 (e.g., Table 3 or 5) differing by no more than 1 (e.g., 0 or 1) nucleotide from the sense strand set forth in any one of Tables 2-5 (e.g., Table 3 or 5).

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence of any one of the sense strands set forth in any one of Tables 2-5 (e.g., Table 3 or 5). In some embodiments, the nucleotide sequence of the sense strand is at least 90%, 91%, 92%, 93%, 94%, 95%, 96%, 97%, 98%, 99% or 100% identical to the nucleotide sequence of any one of the sense strands set forth in any one of Tables 2-5 (e.g., Table 3 or 5). In some embodiments, the nucleotide sequence of the sense strand is at least 95%, 96%, 97%, 98%, 99% or 100% identical to the nucleotide sequence of any one of the sense strands set forth in any one of Tables 2-5 (e.g., Table 3 or 5). In some embodiments, the nucleotide sequence of the sense strand is at least 96%, 97%, 98%, 99% or 100% identical to the nucleotide sequence of any one of the sense strands set forth in any one of Tables 2-5 (e.g., Table 3 or 5). In some embodiments, the nucleotide sequence of the sense strand is at least 97%, identical to the nucleotide sequence of any one of the sense strands set forth in any one of Tables 2-5 (e.g., Table 3 or 5). In some embodiments, the nucleotide sequence of the sense strand is at least 98% identical to the nucleotide sequence of any one of the sense strands set forth in any one of Tables 2-5 (e.g., Table 3 or 5). In some embodiments, the nucleotide sequence of the sense strand is at least 99% identical to the nucleotide sequence of any one of the sense strands set forth in any one of Tables 2-5 (e.g., Table 3 or 5). In some embodiments, the nucleotide sequence of the sense strand is 100% identical to the nucleotide sequence of any one of the sense strands set forth in any one of Tables 2-5 (e.g., Table 3 or 5).

In some embodiments, the nucleotide sequence of the sense strand comprises the nucleotide sequence of any one of the sense strands set forth in any one of Tables 2-5 (e.g., Table 3 or 5). In some embodiments, the nucleotide sequence of the sense strand consists of the nucleotide sequence of any one of the sense strands set forth in any one of Tables 2-5 (e.g., Table 3 or 5).

Tables 2-5 further identify the target nucleic acid molecule within the cited reference BCL11A mRNA transcript (SEQ ID NO: 1) targeted by each of the sense strands. As such, the disclosure further provides sense strands wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence of any one of the sense strands set forth in any one of Tables 2-5 (e.g., Table 3 or 5) and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the BCL11A mRNA transcript targeted by the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence of any one of the sense strands set forth in any one of Tables 2-5 (e.g., Table 3 or 5).

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the sense strand comprises from about 21-23 (e.g., 21-22, 22-23) contiguous nucleotides differing by no more than 3 (e.g., 0, 1, 2, or 3) nucleotides from the nucleotide sequence of any one of the sense strands set forth in any one of Tables 2-5 (e.g., Table 3 or 5).

In some embodiments, the nucleotide sequence of the sense strand comprises the nucleotide sequence of any one of the sense strands set forth in any one of Tables 2-5 (e.g., Table 3 or 5) differing by no more than 3 (e.g., 0, 1, 2, or 3) nucleotides from the sense strand set forth in any one of Tables 2-5 (e.g., Table 3 or 5). In some embodiments, the nucleotide sequence of the sense strand comprises the nucleotide sequence of any one of the sense strands set forth in any one of Tables 2-5 (e.g., Table 3 or 5).

Tables 2-5 further identify the target nucleic acid molecule within the reference BCL11A mRNA transcript at least partially identical to each of the sense strands. As such, the disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in any one of Tables 2-5 (e.g., Table 3 or 5) and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the BCL11A mRNA transcript identical to the select sense strand.

In some embodiments, the nucleotide sequence of the sense strand differs by no more than 5 (e.g., 0, 1, 2, 3, 4, or 5) nucleotides from the nucleotide sequence set forth in any one of SEQ ID NOS: 1577-3166 (e.g., any one of SEQ ID NOS: 1577-1592 or 2346-2361). In some embodiments, the nucleotide sequence of the sense strand differs by no more than 3 (e.g., 0, 1, 2, or 3) nucleotides from the nucleotide sequence set forth in any one of SEQ ID NOS: 1577-3166 (e.g., any one of SEQ ID NOS: 1577-1592 or 2346-2361). In some embodiments, the nucleotide sequence of the sense strand differs by no more than 2 (e.g., 0, 1, or 2) nucleotides from the nucleotide sequence set forth in any one of SEQ ID NOS: 1577-3166 (e.g., any one of SEQ ID NOS: 1577-1592 or 2346-2361). In some embodiments, the nucleotide sequence of the sense strand differs by no more than 1 (e.g., 0 or 1) nucleotide from the nucleotide sequence set forth in any one of SEQ ID NOS: 1577-3166 (e.g., any one of SEQ ID NOS: 1577-1592 or 2346-2361).

In some embodiments, the nucleotide sequence of the sense strand comprises the nucleotide sequence set forth in any one of SEQ ID NOS: 1577-3166 (e.g., any one of SEQ ID NOS: 1577-1592 or 2346-2361) differing by no more than 5 (e.g., 0, 1, 2, 3, 4, or 5) nucleotides from the nucleotide sequence set forth in any one of SEQ ID NOS: 1577-3166 (e.g., any one of SEQ ID NOS: 1577-1592 or 2346-2361). In some embodiments, the nucleotide sequence of the sense strand comprises the nucleotide sequence set forth in any one of SEQ ID NOS: 1577-3166 (e.g., any one of SEQ ID NOS: 1577-1592 or 2346-2361) differing by no more than 3 (e.g., 0, 1, 2, or 3) nucleotides from the nucleotide sequence set forth in any one of SEQ ID NOS: 1577-3166 (e.g., any one of SEQ ID NOS: 1577-1592 or 2346-2361). In some embodiments, the nucleotide sequence of the sense strand comprises the nucleotide sequence set forth in any one of SEQ ID NOS: 1577-3166 (e.g., any one of SEQ ID NOS: 1577-1592 or 2346-2361) differing by no more than 2 (e.g., 0, 1, or 2) nucleotides from the nucleotide sequence set forth in any one of SEQ ID NOS: 1577-3166 (e.g., any one of SEQ ID NOS: 1577-1592 or 2346-2361). In some embodiments, the nucleotide sequence of the sense strand comprises the nucleotide sequence set forth in any one of SEQ ID NOS: 1577-3166 (e.g., any one of SEQ ID NOS: 1577-1592 or 2346-2361) differing by no more than 1 (e.g., 0 or 1) nucleotide from the nucleotide sequence set forth in any one of SEQ ID NOS: 1577-3166 (e.g., any one of SEQ ID NOS: 1577-1592 or 2346-2361).

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in any one of SEQ ID NOS: 1577-3166 (e.g., any one of SEQ ID NOS: 1577-1592 or 2346-2361). In some embodiments, the nucleotide sequence of the sense strand is at least 90%, 91%, 92%, 93%, 94%, 95%, 96%, 97%, 98%, 99% or 100% identical to the nucleotide sequence set forth in any one of SEQ ID NOS: 1577-3166 (e.g., any one of SEQ ID NOS: 1577-1592 or 2346-2361). In some embodiments, the nucleotide sequence of the sense strand is at least 95%, 96%, 97%, 98%, 99% or 100% identical to the nucleotide sequence set forth in any one of SEQ ID NOS: 1577-3166 (e.g., any one of SEQ ID NOS: 1577-1592 or 2346-2361). In some embodiments, the nucleotide sequence of the sense strand is at least 96%, 97%, 98%, 99% or 100% identical to the nucleotide sequence set forth in any one of SEQ ID NOS: 1577-3166 (e.g., any one of SEQ ID NOS: 1577-1592 or 2346-2361). In some embodiments, the nucleotide sequence of the sense strand is at least 97%, identical to the nucleotide sequence set forth in any one of SEQ ID NOS: 1577-3166 (e.g., any one of SEQ ID NOS: 1577-1592 or 2346-2361). In some embodiments, the nucleotide sequence of the sense strand is at least 98% identical to the nucleotide sequence set forth in any one of SEQ ID NOS: 1577-3166 (e.g., any one of SEQ ID NOS: 1577-1592 or 2346-2361). In some embodiments, the nucleotide sequence of the sense strand is at least 99% identical to the nucleotide sequence set forth in any one of SEQ ID NOS: 1577-3166 (e.g., any one of SEQ ID NOS: 1577-1592 or 2346-2361). In some embodiments, the nucleotide sequence of the sense strand is 100% identical to the nucleotide sequence set forth in any one of SEQ ID NOS: 1577-3166 (e.g., any one of SEQ ID NOS: 1577-1592 or 2346-2361).

In some embodiments, the nucleotide sequence of the sense strand comprises the nucleotide sequence set forth in any one of SEQ ID NOS: 1577-3166 (e.g., any one of SEQ ID NOS: 1577-1592 or 2346-2361). In some embodiments, the nucleotide sequence of the sense strand consists of the nucleotide sequence set forth in any one of SEQ ID NOS: 1577-3166 (e.g., any one of SEQ ID NOS: 1577-1592 or 2346-2361).

Tables 2-5 further identify the target nucleic acid molecule within the cited reference BCL11A mRNA transcript (SEQ ID NO: 1) targeted by each of the sense strands. As such, the disclosure further provides sense strands wherein the nucleotide sequence of the sense strand differs by no more than 5 (e.g., 0, 1, 2, 3, 4, or 5 (e.g., no more than 3 (e.g., 0, 1, 2, or 3))) nucleotides from the nucleotide sequence set forth in any one of SEQ ID NOS: 1577-3166 (e.g., any one of SEQ ID NOS: 1577-1592 or 2346-2361) and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the BCL11A mRNA transcript targeted by the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in any one of SEQ ID NOS: 1577-3166 (e.g., any one of SEQ ID NOS: 1577-1592 or 2346-2361). In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands set forth in SEQ ID NOS: 1577-3166. In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence set forth in any one of SEQ ID NOS: 1577-3166 (e.g., any one of SEQ ID NOS: 1577-1592 or 2346-2361). In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence set forth in any one of SEQ ID NOS: 1577-3166 (e.g., any one of SEQ ID NOS: 1577-1592 or 2346-2361).

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in any one of SEQ ID NOS: 1577-3166 (e.g., any one of SEQ ID NOS: 1577-1592 or 2346-2361). In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence set forth in any one of SEQ ID NOS: 1577-3166 (e.g., any one of SEQ ID NOS: 1577-1592 or 2346-2361). In some embodiments, the sense strand comprises from about 21-23 (e.g., 21-22, 22-23) contiguous nucleotides differing by no more than 3 (e.g., 0, 1, 2, or 3) nucleotides from the nucleotide sequence set forth in any one of SEQ ID NOS: 1577-3166 (e.g., any one of SEQ ID NOS: 1577-1592 or 2346-2361).

In some embodiments, the nucleotide sequence of the sense strand comprises the nucleotide sequence set forth in any one of SEQ ID NOS: 1577-3166 (e.g., any one of SEQ ID NOS: 1577-1592 or 2346-2361) differing by no more than 3 (e.g., 0, 1, 2, or 3) nucleotides from the nucleotide sequence set forth in any one of SEQ ID NOS: 1577-3166 (e.g., any one of SEQ ID NOS: 1577-1592 or 2346-2361). In some embodiments, the nucleotide sequence of the sense strand comprises the nucleotide sequence set forth in any one of SEQ ID NOS: 1577-3166 (e.g., any one of SEQ ID NOS: 1577-1592 or 2346-2361).

As described above, Tables 2-5 further identify the target nucleic acid molecule within the reference BCL11A mRNA transcript at least partially identical to each of the sense strands. As such, disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence set forth in any one of SEQ ID NOS: 1577-3166 (e.g., any one of SEQ ID NOS: 1577-1592 or 2346-2361) and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the BCL11A mRNA transcript identical to the select sense strand.

In some embodiments, the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands of any one of dsRNA agents 1-1590 (e.g., one of dsRNA agents 1-16 or 770-785) set forth in any one of Tables 2-5 (e.g., Table 3 or 5). In some embodiments, the sense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands of any one of dsRNA agents 1-1590 (e.g., one of dsRNA agents 1-16 or 770-785) set forth in any one of Tables 2-5 (e.g., Table 3 or 5). In some embodiments, the sense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the sense strands of any one of dsRNA agents 1-1590 (e.g., one of dsRNA agents 1-16 or 770-785) set forth in any one of Tables 2-5 (e.g., Table 3 or 5). In some embodiments, the sense strand comprises at least 23 (e.g., 24, 25, 26, 27, 28, 29, 30) contiguous nucleotides differing by no more than 3 (e.g., 0, 1, 2, or 3) nucleotides from the nucleotide sequence of any one of the sense strands of any one of dsRNA agents 1-1590 (e.g., one of dsRNA agents 1-16 or 770-785) set forth in any one of Tables 2-5 (e.g., Table 3 or 5).

In some embodiments, the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands of any one of dsRNA agents 1-1590 (e.g., one of dsRNA agents 1-16 or 770-785) set forth in any one of Tables 2-5 (e.g., Table 3 or 5). In some embodiments, the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands of any one of dsRNA agents 1-1590 (e.g., one of dsRNA agents 1-16 or 770-785) set forth in any one of Tables 2-5 (e.g., Table 3 or 5). In some embodiments, the sense strand comprises from about 21-23 (e.g., 21-22, 22-23) contiguous nucleotides differing by no more than 3 (e.g., 0, 1, 2, or 3) nucleotides from the nucleotide sequence of any one of the sense strands of any one of dsRNA agents 1-1590 (e.g., one of dsRNA agents 1-16 or 770-785) set forth in any one of Tables 2-5 (e.g., Table 3 or 5).

In some embodiments, the nucleotide sequence of the sense strand comprises the nucleotide sequence of any one of the sense strands of any one of dsRNA agents 1-1590 (e.g., one of dsRNA agents 1-16 or 770-785) set forth in any one of Tables 2-5 (e.g., Table 3 or 5) differing by no more than 3 (e.g., 0, 1, 2, or 3) nucleotides from the sense strand set forth in any one of Tables 2-5 (e.g., Table 3 or 5). In some embodiments, the nucleotide sequence of the sense strand comprises the nucleotide sequence of any one of the sense strands of any one of dsRNA agents 1-1590 (e.g., one of dsRNA agents 1-16 or 770-785) set forth in any one of Tables 2-5 (e.g., Table 3 or 5).

As described above, Tables 2-5 further identify the target nucleic acid molecule within the reference BCL11A mRNA transcript at least partially identical to each of the sense strands. As such, disclosure further provides sense strands comprising at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the sense strands of any one of dsRNA agents 1-1590 (e.g., one of dsRNA agents 1-16 or 770-785) set forth in any one of Tables 2-5 (e.g., Table 3 or 5) and further comprising additional nucleotide sequences (e.g., comprising from about 1-10, 1-9, 1-8, 1-7, 1-6, 1-5, 1-4, 1-3, or 1-2 nucleotides) at least partially identical to the region contiguous (e.g., either at the 3′ end, the 5′ end, or both the 3′ and 5′ end) of the BCL11A mRNA transcript identical to the select sense strand. It is to be understood, that although the sense strands set forth in any one of Tables 2-5 (e.g., Table 3 or 5) are not described as being modified (e.g., comprising chemically modified nucleotides), conjugated, etc., the disclosure includes any sense strand set forth in any one of Tables 2-5 (e.g., Table 3 or 5) that is unmodified, unconjugated, modified (e.g., as described herein), or conjugated (e.g., as described herein).

4.2.3 dsRNA Agents

In some embodiments, the agent (e.g., RNAi agent) comprises a dsRNA agent comprising an antisense strand (e.g., described herein, e.g., described in § 4.2.1) and a sense strand (e.g., described herein, e.g., described in § 4.2.2) that hybridize to form a double stranded region (e.g., under conditions in which the dsRNA will be used (e.g., under physiological (e.g., mammalian, e.g., human) conditions within a cell)).

As described above, antisense strands (e.g., described herein) comprise a region of complementarity that comprises a nucleotide sequence that is at least partially (e.g., substantially, fully) complementary to the nucleotide sequence of a target nucleic acid molecule (e.g., a target mRNA (e.g., a BCL11A mRNA), a portion of a target mRNA (e.g., a BCL11A mRNA)); and the sense strands comprise a region of complementarity that comprises a nucleotide sequence that is at least partially (e.g., substantially, fully) complementary to the nucleotide sequence of at least a portion of an antisense strand.

### 4.2.3.1 Single & Multiple Nucleic Acid Molecules

As described herein, and known in the art, the sense strand and the antisense strand can be part of a single larger nucleic acid molecule (connected as a single stranded nucleic acid molecule) or separate nucleic acid molecules (only connected through the double stranded region). In some embodiments, the sense strand and the antisense strand are separate nucleic acid molecules. In some embodiments, sense strand and the antisense strand are part of a single larger nucleic acid molecule.

In embodiments wherein the sense and antisense strands are part of a single nucleic acid molecule, the nucleic acid molecule may comprise a hairpin loop between the antisense strand and the sense strand to allow for formation of the double stranded region. In some embodiments, the hairpin loop comprises at least 1 (e.g., at least 2, 3, 4, 5, 6, 7, 8, 9, 10, 20, 23, 25 or more) unpaired nucleotides (non-complementary nucleotide mismatches). In some embodiments, the hairpin loop comprises at least one but less than 25, 23, 20, 10, 9, 8, 7, 6, 5, 4, 3, or 2 unpaired nucleotides (non-complementary nucleotide mismatches). In some embodiments, the hairpin loop comprises about 25, 23, 20, 9, 8, 7, 6, 5, 4, 3, or 1 unpaired nucleotide (non-complementary nucleotide mismatch).

Without wishing to be bound by theory, in embodiments wherein the sense strand and the antisense strand are part of a single nucleic acid molecule, after introduction into a suitable cell (e.g., a mammalian cell, e.g., a human cell), the nucleic acid molecule may be cleaved into a dsRNA molecule wherein the two strands of the dsRNA molecule are no longer part of the same nucleic acid molecule e.g., by a Type III endonuclease (e.g., Dicer) (see, e.g., Sharp et al. (2001) Genes Dev. 15:485, the entire contents of which are incorporated by herein by reference for all purposes).

### 4.2.3.2 Length of Double Stranded Region

In some embodiments, the double stranded region is about 15-30 base pairs in length (e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length). In some embodiments, the double stranded region is about 18-25 base pairs in length (e.g., 18-25, 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 base pairs in length (e.g., 19-21 base pairs in length)). In some embodiments, the double stranded region is about 15-30, 15-29, 15-28, 15-27, 15-26, 15-25, 15-24, 15-23, 15-22, 15-21, 15-20, 15-19, 15-18, 15-17, 15-16, 19-30, 19-29, 19-28, 19-27, 19-26, 19-25, 19-24, 19-23, 19-22, 19-20, 19-21, 23-30, 23-29, 23-28, 23-27, 23-26, 23-25, 23-24, 21-30, 21-29, 21-28, 21-27, 21-26, 21-25, 21-24, 21-23, or 21-22 base pairs in length. In some embodiments, the double stranded region is about 19-21 (e.g., 19-20) base pairs in length.

In some embodiments, the double stranded region is not more than about 15, 16, 17, 18, 19, 20, 21, 22, 23, 24, 25, 26, 27, 28, 29, or 30 base pairs in length. In some embodiments, the double stranded region is about 15, 16, 17, 18, 19, 20, 21, 22, 23, 24, 25, 26, 27, 28, 29, or 30 base pairs in length. In some embodiments, the double stranded region is about 19, 20, or 21 base pairs in length. In some embodiments, the double stranded region is about 19 base pairs in length. In some embodiments, the double stranded region is about 20 base pairs in length. In some embodiments, the double stranded region is about 21 base pairs in length. In some embodiments, the double stranded region is about 23 base pairs in length. Ranges and lengths intermediate to the above recited ranges and lengths are also contemplated to be part of the disclosure.

### 4.2.3.3 Nucleotide Overhangs & Blunt Ends

In some embodiments, the dsRNA agent comprises one or more (e.g., 1 or 2) nucleotide overhang. As is clear from the disclosure, but for the sake of clarity, the nucleotides of a nucleotide overhang can include one or more a modified (e.g., chemically modified) nucleotide (e.g., described herein, e.g., described in §§ 4.4, 4.4.1).

In some embodiments, the nucleotide overhang comprises from about 1-5 nucleotides, e.g., 1-4, 1-3, 1-2, 2-5, 2-4, 2-3, 3-5, 3-4, 4-5 nucleotides. In some embodiments, the nucleotide overhang comprises or consists of about 1, 2, 3, 4, or 5 nucleotides. In some embodiments, the nucleotide overhang comprises or consists of about 1 nucleotide. In some embodiments, the nucleotide overhang comprises or consists of about 2 nucleotides.

The nucleotide overhang(s) can be on the sense strand, the antisense strand, or both the sense strand and the antisense strand. In some embodiments, the sense strand comprises a nucleotide overhang. In some embodiments, the antisense strand comprises a nucleotide overhang. In some embodiments, the sense strand and the antisense strand both comprise a nucleotide overhang.

Furthermore, the nucleotide(s) of an overhang can be present on the 5′-end, 3′-end, or both the 5′-end, 3′-end of an antisense or sense strand. In some embodiments, the sense strand comprises a nucleotide overhang at the 5′-end. In some embodiments, the sense strand comprises a nucleotide overhang at the 3′-end. In some embodiments, the sense strand comprises a nucleotide overhang at the 5′-end and the 3′-end. In some embodiments, the antisense strand comprises a nucleotide overhang at the 5′-end. In some embodiments, the antisense strand comprises a nucleotide overhang at the 3′-end. In some embodiments, the antisense strand comprises a nucleotide overhang at the 5′-end and the 3′-end. In some embodiments, the antisense strand comprises a nucleotide overhang at the 3′-end; and the sense strand comprises a nucleotide overhang at the 3′-end. In some embodiments, the antisense strand comprises a nucleotide overhang at the 5′-end; and the sense strand comprises a nucleotide overhang at the 5′-end.

In some embodiments, the dsRNA agent comprises one or more blunt end. In some embodiments, the dsRNA agent comprises a blunt end at the end of the agent comprising the 3′end of the sense strand and the 5′ end of the antisense strand. In some embodiments, the dsRNA agent comprises a blunt end at the end of the agent comprising the 5′end of the sense strand and the 3′ end of the antisense strand. In some embodiments, both ends of the dsRNA agent are blunt ends.

### 4.2.3.4 Exemplary Structural Combinations of Sense & Antisense Strands

In some embodiments, the antisense strand and the sense strand contain the same number of nucleotides. In some embodiments, the antisense strand and the sense strand contain different numbers of nucleotides. In some embodiments, the nucleotide sequence of the sense strand is from about 1-5, 1-3, or 1-2 nucleotides shorter than the nucleotide sequence of the antisense strand. In some embodiments, the nucleotide sequence of the sense strand is about 1, 2, 3, 4, or 5 nucleotides shorter than the nucleotide sequence of the antisense strand. In some embodiments, the nucleotide sequence of the sense strand is about 2 nucleotides shorter than the nucleotide sequence of the antisense strand. In some embodiments, the nucleotide sequence of the antisense strand is from about 1-5, 1-3, or 1-2 nucleotides shorter than the nucleotide sequence of the sense strand. In some embodiments, the nucleotide sequence of the antisense strand is about 1, 2, 3, 4, or 5 nucleotides shorter than the nucleotide sequence of the sense strand. In some embodiments, the nucleotide sequence of the antisense strand is about 2 nucleotides shorter than the nucleotide sequence of the sense strand.

In some embodiments, the sense strand comprises or consists of 21 nucleotides. In some embodiments, the antisense strand comprises or consists of 23 nucleotides. In some embodiments, the sense strand comprises or consists of 21 nucleotides; and the antisense strand comprises or consists of 23 nucleotides. In some preferred embodiments, the sense strand comprises 21 nucleotides; and the antisense strand comprises 23 nucleotides. In some preferred embodiments, the sense strand consists of 21 nucleotides; and the antisense strand consists of 23 nucleotides. In some embodiments, the double stranded region comprises or consists of 21 nucleotides. In some embodiments, the antisense strand comprises a 2-nucleotide overhang at the 3′end. In some embodiments, the 5′ end of the antisense strand and 3′ end of the sense strand form a blunt end. In some embodiments, the sense strand comprises or consists of 21 nucleotides; the antisense strand comprises or consists of 23 nucleotides; the double stranded region comprises or consists of 21 nucleotides; the antisense strand comprises a 2-nucleotide overhang at the 3′end; and the 5′ end of the antisense strand and 3′ end of the sense strand form a blunt end.

In some embodiments, the sense strand comprises or consists of 19 nucleotides. In some embodiments, the antisense strand comprises or consists of 21 nucleotides. In some embodiments, the sense strand comprises or consists of 19 nucleotides; and the antisense strand comprises or consists of 21 nucleotides. In some embodiments, the double stranded region comprises or consists of 19 nucleotides. In some embodiments, the antisense strand comprises a 2-nucleotide overhang at the 3′end. In some embodiments, the 5′ end of the antisense strand and 3′ end of the sense strand form a blunt end. In some embodiments, the sense strand comprises or consists of 19 nucleotides; the antisense strand comprises or consists of 21 nucleotides; the double stranded region comprises or consists of 19 nucleotides; the antisense strand comprises a 2-nucleotide overhang at the 3′end; and the 5′ end of the antisense strand and 3′ end of the sense strand form a blunt end.

In some embodiments, the sense strand comprises or consists of 21 nucleotides. In some embodiments, the antisense strand comprises or consists of 21 nucleotides. In some embodiments, the sense strand comprises or consists of 21 nucleotides; and the antisense strand comprises or consists of 21 nucleotides. In some embodiments, the double stranded region comprises or consists of 19 nucleotides. In some embodiments, the antisense strand comprises a 2-nucleotide overhang at the 3′end. In some embodiments, the sense strand comprises a 2-nucleotide overhang at the 3′end. In some embodiments, the sense strand comprises or consists of 21 nucleotides; the antisense strand comprises or consists of 21 nucleotides; the double stranded region comprises or consists of 19 nucleotides; the antisense strand comprises a 2-nucleotide overhang at the 3′end; and the sense strand comprises a 2-nucleotide overhang at the 3′end.

In some embodiments, the sense strand comprises or consists of 20 nucleotides. In some embodiments, the antisense strand comprises or consists of 19 nucleotides. In some embodiments, the sense strand comprises or consists of 20 nucleotides; and the antisense strand comprises or consists of 19 nucleotides. In some embodiments, the double stranded region comprises or consists of 20 nucleotides. In some embodiments, the sense strand comprises a 1-nucleotide overhang at the 5′ end. In some embodiments, the 5′ end of the antisense strand and 3′ end of the sense strand form a blunt end. In some embodiments, the sense strand comprises or consists of 20 nucleotides; the antisense strand comprises or consists of 19 nucleotides; the double stranded region comprises or consists of 20 nucleotides; the sense strand comprises a 1-nucleotide overhang at the 5′end; and the 5′ end of the antisense strand and 3′ end of the sense strand form a blunt end.

In some embodiments, the sense strand comprises or consists of 21 nucleotides. In some embodiments, the antisense strand comprises or consists of 19 nucleotides. In some embodiments, the sense strand comprises or consists of 21 nucleotides; and the antisense strand comprises or consists of 19 nucleotides. In some embodiments, the double stranded region comprises or consists of 19 nucleotides. In some embodiments, the sense strand comprises a 1-nucleotide overhang at the 3′end. In some embodiments, the sense strand comprises a 1-nucleotide overhang at the 5′end. In some embodiments, the sense strand comprises or consists of 21 nucleotides; the antisense strand comprises or consists of 19 nucleotides; the double stranded region comprises or consists of 19 nucleotides; the sense strand comprises a 1-nucleotide overhang at the 3′end; and the sense strand comprises a 1-nucleotide overhang at the 5′ end.

In some embodiments, the sense strand comprises or consists of 24 nucleotides. In some embodiments, the antisense strand comprises or consists of 23 nucleotides. In some embodiments, the sense strand comprises or consists of 24 nucleotides; and the antisense strand comprises or consists of 23 nucleotides. In some embodiments, the double stranded region comprises or consists of 21 nucleotides. In some embodiments, the antisense strand comprises a 2-nucleotide overhang at the 3′end. In some embodiments, the sense strand comprises a 3-nucleotide overhang at the 3′end. In some embodiments, the sense strand comprises or consists of 24 nucleotides; the antisense strand comprises or consists of 23 nucleotides; the double stranded region comprises or consists of 21 nucleotides; the antisense strand comprises a 2-nucleotide overhang at the 3′end; and the sense strand comprises a 3-nucleotide overhang at the 3′ end.

In some embodiments, the sense strand comprises or consists of 19 nucleotides. In some embodiments, the antisense strand comprises or consists of 19 nucleotides. In some embodiments, the sense strand comprises or consists of 19 nucleotides; and the antisense strand comprises or consists of 19 nucleotides. In some embodiments, the double stranded region comprises or consists of 19 nucleotides. In some embodiments, the 5′ end of the antisense strand (and 3′ end of the sense strand) form a blunt end. In some embodiments, the 3′ end of the antisense strand (and 5′ end of the sense strand) form a blunt end. In some embodiments, the sense strand comprises or consists of 19 nucleotides; the antisense strand comprises or consists of 19 nucleotides; the double stranded region comprises or consists of 19 nucleotides; the 5′ end of the antisense strand (and 3′ end of the sense strand) form a blunt end; and the 3′ end of the antisense strand (and 5′ end of the sense strand) form a blunt end

In some embodiments, the antisense strand and the sense strand are part of the same larger nucleic acid molecule, wherein the nucleic acid molecule comprises or consists of 44 nucleotides, the antisense portion comprises or consists of 21 nucleotides, the sense strand portion of the nucleic acid molecule comprises 19 nucleotides, the double stranded region comprises or consists of 19 nucleotides, the antisense strand comprises a 2-nucleotide overhang at the 3′end, and the intervening nucleotide sequence between the antisense strand and the sense strand comprises or consists of 4 unpaired nucleotides that create a hairpin loop.

### 4.2.3.5 Exemplary Antisense Strands & Sense Strands

In some embodiments, the antisense strand is an antisense strand described herein. In some embodiments, the sense strand is a sense strand described herein. In some embodiments, the antisense strand is an antisense strand described in § 4.2.1. In some embodiments, the sense strand is a sense strand described in § 4.2.2. In some embodiments, the antisense strand is an antisense strand described in § 4.2.1; and the sense strand is a sense strand described in § 4.2.2. It is to be understood that any sense strand described herein (e.g., in § 4.2.2); and be utilized in combination with any antisense strand in a dsRNA agent described herein (e.g., in § 4.2.1). For the sake of clarity, the entire contents of in §§ 4.2.1 and § 4.2.2, are incorporated by reference into the instant section in § 4.2.3.5.

In some embodiments, the nucleotide sequence of the sense strand differs by no more than 5 (e.g., 0, 1, 2, 3, 4, or 5) nucleotides from the nucleotide sequence of any one of the sense strands set forth in any one of Tables 2-5 (e.g., Table 3 or 5); and the nucleotide sequence of the antisense strand differs by no more than 5 (e.g., 0, 1, 2, 3, 4, or 5) nucleotides from the nucleotide sequence of any one of the antisense strands set forth in any one of Tables 2-5 (e.g., Table 3 or 5). In some embodiments, the nucleotide sequence of the sense strand differs by no more than 3 (e.g., 0, 1, 2, or 3) nucleotides from the nucleotide sequence of any one of the sense strands set forth in any one of Tables 2-5 (e.g., Table 3 or 5); and the nucleotide sequence of the antisense strand differs by no more than 3 (e.g., 0, 1, 2, or 3) nucleotides from the nucleotide sequence of any one of the antisense strands set forth in any one of Tables 2-5 (e.g., Table 3 or 5). In some embodiments, the nucleotide sequence of the sense strand differs by no more than 2 (e.g., 0, 1, or 2) nucleotides from the nucleotide sequence of any one of the sense strands set forth in any one of Tables 2-5 (e.g., Table 3 or 5); and the nucleotide sequence of the antisense strand differs by no more than 2 (e.g., 0, 1, or 2) nucleotides from the nucleotide sequence of any one of the antisense strands set forth in any one of Tables 2-5 (e.g., Table 3 or 5). In some embodiments, the nucleotide sequence of the sense strand differs by no more than 1 (e.g., 0 or 1) nucleotide from the nucleotide sequence of any one of the sense strands set forth in any one of Tables 2-5 (e.g., Table 3 or 5); and the nucleotide sequence of the antisense strand differs by no more than 1 (e.g., 0 or 1) nucleotide from the nucleotide sequence of any one of the antisense strands set forth in any one of Tables 2-5 (e.g., Table 3 or 5).

In some embodiments, the nucleotide sequence of the sense strand comprises the nucleotide sequence of any one of the sense strands set forth in any one of Tables 2-5 (e.g., Table 3 or 5) differing by no more than 5 (e.g., 0, 1, 2, 3, 4, or 5) nucleotides from the sense strand set forth in any one of Tables 2-5 (e.g., Table 3 or 5); and the nucleotide sequence of the antisense strand comprises the nucleotide sequence of any one of the antisense strands set forth in any one of Tables 2-5 (e.g., Table 3 or 5) differing by no more than 5 (e.g., 0, 1, 2, 3, 4, or 5) nucleotides from the antisense strand set forth in any one of Tables 2-5 (e.g., Table 3 or 5). In some embodiments, the nucleotide sequence of the sense strand comprises the nucleotide sequence of any one of the sense strands set forth in any one of Tables 2-5 (e.g., Table 3 or 5) differing by no more than 3 (e.g., 0, 1, 2, or 3) nucleotides from the sense strand set forth in any one of Tables 2-5 (e.g., Table 3 or 5); and the nucleotide sequence of the antisense strand comprises the nucleotide sequence of any one of the antisense strands set forth in any one of Tables 2-5 (e.g., Table 3 or 5) differing by no more than 3 (e.g., 0, 1, 2, or 3) nucleotides from the antisense strand set forth in any one of Tables 2-5 (e.g., Table 3 or 5). In some embodiments, the nucleotide sequence of the sense strand comprises the nucleotide sequence of any one of the sense strands set forth in any one of Tables 2-5 (e.g., Table 3 or 5) differing by no more than 2 (e.g., 0, 1, or 2) nucleotides from the sense strand set forth in any one of Tables 2-5 (e.g., Table 3 or 5); and the nucleotide sequence of the antisense strand comprises the nucleotide sequence of any one of the antisense strands set forth in any one of Tables 2-5 (e.g., Table 3 or 5) differing by no more than 2 (e.g., 0, 1, or 2) nucleotides from the antisense strand set forth in any one of Tables 2-5 (e.g., Table 3 or 5). In some embodiments, the nucleotide sequence of the sense strand comprises the nucleotide sequence of any one of the sense strands set forth in any one of Tables 2-5 (e.g., Table 3 or 5) differing by no more than 1 (e.g., 0 or 1) nucleotide from the sense strand set forth in any one of Tables 2-5 (e.g., Table 3 or 5); and the nucleotide sequence of the antisense strand comprises the nucleotide sequence of any one of the antisense strands set forth in any one of Tables 2-5 (e.g., Table 3 or 5) differing by no more than 1 (e.g., 0 or 1) nucleotide from the antisense strand set forth in any one of Tables 2-5 (e.g., Table 3 or 5).

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence of any one of the sense strands set forth in any one of Tables 2-5 (e.g., Table 3 or 5); and the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence of any one of the antisense strands set forth in any one of Tables 2-5 (e.g., Table 3 or 5). In some embodiments, the nucleotide sequence of the sense strand is at least 90%, 91%, 92%, 93%, 94%, 95%, 96%, 97%, 98%, 99% or 100% identical to the nucleotide sequence of any one of the sense strands set forth in any one of Tables 2-5 (e.g., Table 3 or 5); and the nucleotide sequence of the antisense strand is at least 90%, 91%, 92%, 93%, 94%, 95%, 96%, 97%, 98%, 99% or 100% identical to the nucleotide sequence of any one of the antisense strands set forth in any one of Tables 2-5 (e.g., Table 3 or 5). In some embodiments, the nucleotide sequence of the sense strand is at least 95%, 96%, 97%, 98%, 99% or 100% identical to the nucleotide sequence of any one of the sense strands set forth in any one of Tables 2-5 (e.g., Table 3 or 5); and the nucleotide sequence of the antisense strand is at least 95%, 96%, 97%, 98%, 99% or 100% identical to the nucleotide sequence of any one of the antisense strands set forth in any one of Tables 2-5 (e.g., Table 3 or 5). In some embodiments, the nucleotide sequence of the sense strand is at least 96%, 97%, 98%, 99% or 100% identical to the nucleotide sequence of any one of the sense strands set forth in any one of Tables 2-5 (e.g., Table 3 or 5); and the nucleotide sequence of the antisense strand is at least 96%, 97%, 98%, 99% or 100% identical to the nucleotide sequence of any one of the antisense strands set forth in any one of Tables 2-5 (e.g., Table 3 or 5). In some embodiments, the nucleotide sequence of the sense strand is at least 97%, identical to the nucleotide sequence of any one of the sense strands set forth in any one of Tables 2-5 (e.g., Table 3 or 5); and the nucleotide sequence of the antisense strand is at least 97%, identical to the nucleotide sequence of any one of the antisense strands set forth in any one of Tables 2-5 (e.g., Table 3 or 5). In some embodiments, the nucleotide sequence of the sense strand is at least 98% identical to the nucleotide sequence of any one of the sense strands set forth in any one of Tables 2-5 (e.g., Table 3 or 5); and the nucleotide sequence of the antisense strand is at least 98% identical to the nucleotide sequence of any one of the antisense strands set forth in any one of Tables 2-5 (e.g., Table 3 or 5). In some embodiments, the nucleotide sequence of the sense strand is at least 99% identical to the nucleotide sequence of any one of the sense strands set forth in any one of Tables 2-5 (e.g., Table 3 or 5); and the nucleotide sequence of the antisense strand is at least 99% identical to the nucleotide sequence of any one of the antisense strands set forth in any one of Tables 2-5 (e.g., Table 3 or 5). In some embodiments, the nucleotide sequence of the sense strand is 100% identical to the nucleotide sequence of any one of the sense strands set forth in any one of Tables 2-5 (e.g., Table 3 or 5); and the nucleotide sequence of the antisense strand is 100% identical to the nucleotide sequence of any one of the antisense strands set forth in any one of Tables 2-5 (e.g., Table 3 or 5).

In some embodiments, the nucleotide sequence of the sense strand comprises the nucleotide sequence of any one of the sense strands set forth in any one of Tables 2-5 (e.g., Table 3 or 5); and the nucleotide sequence of the antisense strand comprises the nucleotide sequence of any one of the antisense strands set forth in any one of Tables 2-5 (e.g., Table 3 or 5). In some embodiments, the nucleotide sequence of the sense strand consists of the nucleotide sequence of any one of the sense strands set forth in any one of Tables 2-5 (e.g., Table 3 or 5); and the nucleotide sequence of the antisense strand consists of the nucleotide sequence of any one of the antisense strands set forth in any one of Tables 2-5 (e.g., Table 3 or 5).

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of any one of the sense strands set forth in any one of Tables 2-5 (e.g., Table 3 or 5).

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises from about 21-23 (e.g., 21-22, 22-23) contiguous nucleotides differing by no more than 3 (e.g., 0, 1, 2, or 3) nucleotides from the nucleotide sequence of any one of the sense strands set forth in any one of Tables 2-5 (e.g., Table 3 or 5).

In some embodiments, the antisense strand comprises or consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises or consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the antisense strand comprises or consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises or consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises or consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises or consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the antisense strand comprises or consists of about 23, 24, 25, 26, 27, 28, 29, 30 (e.g., 23) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises or consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the antisense strand comprises or consists of about 23 contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises or consists of about 21 contiguous nucleotides differing by no more than 3 (e.g., 0, 1, 2, or 3) nucleotides from the nucleotide sequence of any one of the sense strands set forth in any one of Tables 2-5 (e.g., Table 3 or 5). In some embodiments, the antisense strand comprises or consists of about 21 contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises or consists of about 19 contiguous nucleotides differing by no more than 3 (e.g., 0, 1, 2, or 3) nucleotides from the nucleotide sequence of any one of the sense strands set forth in any one of Tables 2-5 (e.g., Table 3 or 5).

In some embodiments, the nucleotide sequence of the antisense strand comprises or consists of the nucleotide sequence of any one of the antisense strands set forth in any one of Tables 2-5 (e.g., Table 3 or 5) differing by no more than 3 (e.g., 0, 1, 2, or 3) nucleotides from the antisense strand set forth in any one of Tables 2-5 (e.g., Table 3 or 5); and the nucleotide sequence of the sense strand comprises or consists of the nucleotide sequence of any one of the sense strands set forth in any one of Tables 2-5 (e.g., Table 3 or 5) differing by no more than 3 (e.g., 0, 1, 2, or 3) nucleotides from the sense strand set forth in any one of Tables 2-5 (e.g., Table 3 or 5).

In some embodiments, the nucleotide sequence of the antisense strand comprises or consists of the nucleotide sequence of any one of any one of the antisense strands set forth in any one of Tables 2-5 (e.g., Table 3 or 5); and the nucleotide sequence of the sense strand comprises or consists of the nucleotide sequence of any one of the sense strands set forth in any one of Tables 2-5 (e.g., Table 3 or 5).

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the corresponding sense strand set forth in any one of Tables 2-5 (e.g., Table 3 or 5). In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the corresponding sense strand set forth in any one of Tables 2-5 (e.g., Table 3 or 5). In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the corresponding sense strand set forth in any one of Tables 2-5 (e.g., Table 3 or 5). In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the corresponding sense strand set forth in any one of Tables 2-5 (e.g., Table 3 or 5). In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the corresponding sense strand set forth in any one of Tables 2-5 (e.g., Table 3 or 5).

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the corresponding sense strand set forth in any one of Tables 2-5 (e.g., Table 3 or 5). In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the corresponding sense strand set forth in any one of Tables 2-5 (e.g., Table 3 or 5). In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the corresponding sense strand set forth in any one of Tables 2-5 (e.g., Table 3 or 5). In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises from about 21-23 (e.g., 21-22, 22-23) contiguous nucleotides differing by no more than 3 (e.g., 0, 1, 2, or 3) nucleotides from the nucleotide sequence of the corresponding sense strand set forth in any one of Tables 2-5 (e.g., Table 3 or 5).

In some embodiments, the antisense strand comprises or consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises or consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the corresponding sense strand set forth in any one of Tables 2-5 (e.g., Table 3 or 5). In some embodiments, the antisense strand comprises or consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises or consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the corresponding sense strand set forth in any one of Tables 2-5 (e.g., Table 3 or 5). In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises or consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the corresponding sense strand set forth in any one of Tables 2-5 (e.g., Table 3 or 5). In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises or consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the corresponding sense strand set forth in any one of Tables 2-5 (e.g., Table 3 or 5). In some embodiments, the antisense strand comprises or consists of about 23, 24, 25, 26, 27, 28, 29, 30 (e.g., 23) contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises or consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the corresponding sense strand set forth in any one of Tables 2-5 (e.g., Table 3 or 5). In some embodiments, the antisense strand comprises or consists of about 23 contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises or consists of about 21 contiguous nucleotides differing by no more than 3 (e.g., 0, 1, 2, or 3) nucleotides from the nucleotide sequence of the corresponding sense strand set forth in any one of Tables 2-5 (e.g., Table 3 or 5). In some embodiments, the antisense strand comprises or consists of about 21 contiguous nucleotides differing by no more than 3 (e.g., 0, 1, 2, or 3) nucleotides from the nucleotide sequence of any one of the antisense strands set forth in any one of Tables 2-5 (e.g., Table 3 or 5); and the sense strand comprises or consists of about 19 contiguous nucleotides differing by no more than 3 (e.g., 0, 1, 2, or 3) nucleotides from the nucleotide sequence of the corresponding sense strand set forth in any one of Tables 2-5 (e.g., Table 3 or 5).

In some embodiments, the nucleotide sequence of the antisense strand comprises or consists of the nucleotide sequence of any one of the antisense strands set forth in any one of Tables 2-5 (e.g., Table 3 or 5) differing by no more than 3 (e.g., 0, 1, 2, or 3) nucleotides from the antisense strand set forth in any one of Tables 2-5 (e.g., Table 3 or 5); and the nucleotide sequence of the sense strand comprises or consists of the nucleotide sequence of the corresponding sense strand set forth in any one of Tables 2-5 (e.g., Table 3 or 5) differing by no more than 3 (e.g., 0, 1, 2, or 3) nucleotides from the corresponding sense strand set forth in any one of Tables 2-5 (e.g., Table 3 or 5).

In some embodiments, the nucleotide sequence of the antisense strand comprises or consists of the nucleotide sequence of any one of any one of the antisense strands set forth in any one of Tables 2-5 (e.g., Table 3 or 5); and the nucleotide sequence of the sense strand comprises or consists of the nucleotide sequence of the corresponding sense strand set forth in any one of Tables 2-5 (e.g., Table 3 or 5).

In some embodiments, the nucleotide sequence of the sense strand differs by no more than 5 (e.g., 0, 1, 2, 3, 4, or 5) nucleotides from the nucleotide sequence set forth in any one of SEQ ID NOS: 1577-3166 (e.g., any one of SEQ ID NOS: 1577-1592 or 2346-2361); and the nucleotide sequence of the antisense strand comprises the nucleotide sequence set forth in any one of SEQ ID NOS: 4-1576 (e.g., any one of SEQ ID NOS: 4-19 or 772-787) differing by no more than 5 (e.g., 0, 1, 2, 3, 4, or 5) nucleotides from the nucleotide sequence set forth in any one of SEQ ID NOS: 4-1576 (e.g., any one of SEQ ID NOS: 4-19 or 772-787). In some embodiments, the nucleotide sequence of the sense strand differs by no more than 3 (e.g., 0, 1, 2, or 3) nucleotides from the nucleotide sequence set forth in any one of SEQ ID NOS: 1577-3166 (e.g., any one of SEQ ID NOS: 1577-1592 or 2346-2361); and the nucleotide sequence of the antisense strand comprises the nucleotide sequence set forth in any one of SEQ ID NOS: 4-1576 (e.g., any one of SEQ ID NOS: 4-19 or 772-787) differing by no more than 3 (e.g., 0, 1, 2, or 3) nucleotides from the nucleotide sequence set forth in any one of SEQ ID NOS: 4-1576 (e.g., any one of SEQ ID NOS: 4-19 or 772-787). In some embodiments, the nucleotide sequence of the sense strand differs by no more than 2 (e.g., 0, 1, or 2) nucleotides from the nucleotide sequence set forth in any one of SEQ ID NOS: 1577-3166 (e.g., any one of SEQ ID NOS: 1577-1592 or 2346-2361); and the nucleotide sequence of the antisense strand comprises the nucleotide sequence set forth in any one of SEQ ID NOS: 4-1576 (e.g., any one of SEQ ID NOS: 4-19 or 772-787) differing by no more than 2 (e.g., 0, 1, or 2) nucleotides from the nucleotide sequence set forth in any one of SEQ ID NOS: 4-1576 (e.g., any one of SEQ ID NOS: 4-19 or 772-787). In some embodiments, the nucleotide sequence of the sense strand differs by no more than 1 (e.g., 0 or 1) nucleotide from the nucleotide sequence set forth in any one of SEQ ID NOS: 1577-3166 (e.g., any one of SEQ ID NOS: 1577-1592 or 2346-2361); and the nucleotide sequence of the antisense strand comprises the nucleotide sequence set forth in any one of SEQ ID NOS: 4-1576 (e.g., any one of SEQ ID NOS: 4-19 or 772-787) differing by no more than 1 (e.g., 0 or 1) nucleotide from the nucleotide sequence set forth in any one of SEQ ID NOS: 4-1576 (e.g., any one of SEQ ID NOS: 4-19 or 772-787).

In some embodiments, the nucleotide sequence of the sense strand comprises the nucleotide sequence set forth in any one of SEQ ID NOS: 1577-3166 (e.g., any one of SEQ ID NOS: 1577-1592 or 2346-2361) differing by no more than 5 (e.g., 0, 1, 2, 3, 4, or 5) nucleotides from the nucleotide sequence set forth in any one of SEQ ID NOS: 1577-3166 (e.g., any one of SEQ ID NOS: 1577-1592 or 2346-2361); and the nucleotide sequence of the antisense strand differs by no more than 5 (e.g., 0, 1, 2, 3, 4, or 5) nucleotides from the nucleotide sequence set forth in any one of SEQ ID NOS: 4-1576 (e.g., any one of SEQ ID NOS: 4-19 or 772-787). In some embodiments, the nucleotide sequence of the sense strand comprises the nucleotide sequence set forth in any one of SEQ ID NOS: 1577-3166 (e.g., any one of SEQ ID NOS: 1577-1592 or 2346-2361) differing by no more than 3 (e.g., 0, 1, 2, or 3) nucleotides from the nucleotide sequence set forth in any one of SEQ ID NOS: 1577-3166 (e.g., any one of SEQ ID NOS: 1577-1592 or 2346-2361); and the nucleotide sequence of the antisense strand differs by no more than 3 (e.g., 0, 1, 2, or 3) nucleotides from the nucleotide sequence set forth in any one of SEQ ID NOS: 4-1576 (e.g., any one of SEQ ID NOS: 4-19 or 772-787). In some embodiments, the nucleotide sequence of the sense strand comprises the nucleotide sequence set forth in any one of SEQ ID NOS: 1577-3166 (e.g., any one of SEQ ID NOS: 1577-1592 or 2346-2361) differing by no more than 2 (e.g., 0, 1, or 2) nucleotides from the nucleotide sequence set forth in any one of SEQ ID NOS: 1577-3166 (e.g., any one of SEQ ID NOS: 1577-1592 or 2346-2361); and the nucleotide sequence of the antisense strand differs by no more than 2 (e.g., 0, 1, or 2) nucleotides from the nucleotide sequence set forth in any one of SEQ ID NOS: 4-1576 (e.g., any one of SEQ ID NOS: 4-19 or 772-787). In some embodiments, the nucleotide sequence of the sense strand comprises the nucleotide sequence set forth in any one of SEQ ID NOS: 1577-3166 (e.g., any one of SEQ ID NOS: 1577-1592 or 2346-2361) differing by no more than 1 (e.g., 0 or 1) nucleotide from the nucleotide sequence set forth in any one of SEQ ID NOS: 1577-3166 (e.g., any one of SEQ ID NOS: 1577-1592 or 2346-2361); and the nucleotide sequence of the antisense strand differs by no more than 1 (e.g., 0 or 1) nucleotide from the nucleotide sequence set forth in any one of SEQ ID NOS: 4-1576 (e.g., any one of SEQ ID NOS: 4-19 or 772-787).

In some embodiments, the nucleotide sequence of the sense strand is at least 85%, 86%, 87%, 88%, 89%, 90%, 91%, 92%, 93%, 94%, 95%, 96%, 97%, 98%, 99% or 100% identical to the nucleotide sequence set forth in any one of SEQ ID NOS: 1577-3166 (e.g., any one of SEQ ID NOS: 1577-1592 or 2346-2361); and the nucleotide sequence of the antisense strand is at least 85%, 86%, 87%, 88%, 89%, 90%, 91%, 92%, 93%, 94%, 95%, 96%, 97%, 98%, 99% or 100% identical to the nucleotide sequence set forth in any one of SEQ ID NOS: 4-1576 (e.g., any one of SEQ ID NOS: 4-19 or 772-787). In some embodiments, the nucleotide sequence of the sense strand is at least 90%, 91%, 92%, 93%, 94%, 95%, 96%, 97%, 98%, 99% or 100% identical to the nucleotide sequence set forth in any one of SEQ ID NOS: 1577-3166 (e.g., any one of SEQ ID NOS: 1577-1592 or 2346-2361); and the nucleotide sequence of the antisense strand is at least 90%, 91%, 92%, 93%, 94%, 95%, 96%, 97%, 98%, 99% or 100% identical to the nucleotide sequence set forth in any one of SEQ ID NOS: 4-1576 (e.g., any one of SEQ ID NOS: 4-19 or 772-787). In some embodiments, the nucleotide sequence of the sense strand is at least 95%, 96%, 97%, 98%, 99% or 100% identical to the nucleotide sequence set forth in any one of SEQ ID NOS: 1577-3166 (e.g., any one of SEQ ID NOS: 1577-1592 or 2346-2361); and the nucleotide sequence of the antisense strand is at least 95%, 96%, 97%, 98%, 99% or 100% identical to the nucleotide sequence set forth in any one of SEQ ID NOS: 4-1576 (e.g., any one of SEQ ID NOS: 4-19 or 772-787). In some embodiments, the nucleotide sequence of the sense strand is at least 96%, 97%, 98%, 99% or 100% identical to the nucleotide sequence set forth in any one of SEQ ID NOS: 1577-3166 (e.g., any one of SEQ ID NOS: 1577-1592 or 2346-2361); and the nucleotide sequence of the antisense strand is at least 96%, 97%, 98%, 99% or 100% identical to the nucleotide sequence set forth in any one of SEQ ID NOS: 4-1576 (e.g., any one of SEQ ID NOS: 4-19 or 772-787). In some embodiments, the nucleotide sequence of the sense strand is at least 97%, identical to the nucleotide sequence set forth in any one of SEQ ID NOS: 1577-3166 (e.g., any one of SEQ ID NOS: 1577-1592 or 2346-2361); and the nucleotide sequence of the antisense strand is at least 97%, identical to the nucleotide sequence set forth in any one of SEQ ID NOS: 4-1576 (e.g., any one of SEQ ID NOS: 4-19 or 772-787). In some embodiments, the nucleotide sequence of the sense strand is at least 98% identical to the nucleotide sequence set forth in any one of SEQ ID NOS: 1577-3166 (e.g., any one of SEQ ID NOS: 1577-1592 or 2346-2361); and the nucleotide sequence of the antisense strand is at least 98% identical to the nucleotide sequence set forth in any one of SEQ ID NOS: 4-1576 (e.g., any one of SEQ ID NOS: 4-19 or 772-787). In some embodiments, the nucleotide sequence of the sense strand is at least 99% identical to the nucleotide sequence set forth in any one of SEQ ID NOS: 1577-3166 (e.g., any one of SEQ ID NOS: 1577-1592 or 2346-2361); and the nucleotide sequence of the antisense strand is at least 99% identical to the nucleotide sequence set forth in any one of SEQ ID NOS: 4-1576 (e.g., any one of SEQ ID NOS: 4-19 or 772-787). In some embodiments, the nucleotide sequence of the sense strand is 100% identical to the nucleotide sequence set forth in any one of SEQ ID NOS: 1577-3166 (e.g., any one of SEQ ID NOS: 1577-1592 or 2346-2361); and the nucleotide sequence of the antisense strand is 100% identical to the nucleotide sequence set forth in any one of SEQ ID NOS: 4-1576 (e.g., any one of SEQ ID NOS: 4-19 or 772-787).

In some embodiments, the nucleotide sequence of the sense strand comprises the nucleotide sequence set forth in any one of SEQ ID NOS: 1577-3166 (e.g., any one of SEQ ID NOS: 1577-1592 or 2346-2361); and the nucleotide sequence of the antisense strand comprises the nucleotide sequence set forth in any one of SEQ ID NOS: 4-1576 (e.g., any one of SEQ ID NOS: 4-19 or 772-787). In some embodiments, the nucleotide sequence of the sense strand consists of the nucleotide sequence set forth in any one of SEQ ID NOS: 1577-3166 (e.g., any one of SEQ ID NOS: 1577-1592 or 2346-2361); and the nucleotide sequence of the antisense strand consists of the nucleotide sequence set forth in any one of SEQ ID NOS: 4-1576 (e.g., any one of SEQ ID NOS: 4-19 or 772-787).

In some embodiments, the nucleotide sequence of the sense strand differs by no more than 5 (e.g., 0, 1, 2, 3, 4, or 5) nucleotides from the nucleotide sequence set forth in any one of SEQ ID NOS: 1577-3166 (e.g., any one of SEQ ID NOS: 1577-1592 or 2346-2361). In some embodiments, the nucleotide sequence of the sense strand differs by no more than 3 (e.g., 0, 1, 2, or 3) nucleotides from the nucleotide sequence set forth in any one of SEQ ID NOS: 1577-3166 (e.g., any one of SEQ ID NOS: 1577-1592 or 2346-2361). In some embodiments, the nucleotide sequence of the sense strand differs by no more than 2 (e.g., 0, 1, or 2) nucleotides from the nucleotide sequence set forth in any one of SEQ ID NOS: 1577-3166 (e.g., any one of SEQ ID NOS: 1577-1592 or 2346-2361). In some embodiments, the nucleotide sequence of the sense strand differs by no more than 1 (e.g., 0 or 1) nucleotide from the nucleotide sequence set forth in any one of SEQ ID NOS: 1577-3166 (e.g., any one of SEQ ID NOS: 1577-1592 or 2346-2361).

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361). In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361). In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361). In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361). In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361).

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361). In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361). In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361). In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises from about 21-23 (e.g., 21-22, 22-23) contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361).

In some embodiments, the antisense strand comprises or consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises or consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361). In some embodiments, the antisense strand comprises or consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises or consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361). In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises or consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361). In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises or consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361). In some embodiments, the antisense strand comprises or consists of about 23, 24, 25, 26, 27, 28, 29, 30 (e.g., 23) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises or consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361). In some embodiments, the antisense strand comprises or consists of about 23 contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises or consists of about 21 contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361). In some embodiments, the antisense strand comprises or consists of about 21 contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises or consists of about 19 contiguous nucleotides differing by no more than 3 (e.g., 0, 1, 2, or 3) nucleotides from the nucleotide sequence of any one of the sense strands set forth in any one of SEQ ID NOS: 1577-3166 (e.g., any one of SEQ ID NOS: 1577-1592 or 2346-2361).

In some embodiments, the nucleotide sequence of the antisense strand comprises or consists of the nucleotide sequence of any one of the antisense strands set forth in any one of SEQ ID NOS: 4-1576 (e.g., any one of SEQ ID NOS: 4-19 or 772-787) differing by no more than 3 (e.g., 0, 1, 2, or 3) nucleotides from the antisense strand set forth in any one of SEQ ID NOS: 4-1576 (e.g., any one of SEQ ID NOS: 4-19 or 772-787); and the nucleotide sequence of the sense strand comprises or consists of the nucleotide sequence of any one of the sense strands set forth in any one of SEQ ID NOS: 1577-3166 (e.g., any one of SEQ ID NOS: 1577-1592 or 2346-2361) differing by no more than 3 (e.g., 0, 1, 2, or 3) nucleotides from the sense strand set forth in any one of SEQ ID NOS: 1577-3166 (e.g., any one of SEQ ID NOS: 1577-1592 or 2346-2361).

In some embodiments, the nucleotide sequence of the antisense strand comprises or consists of the nucleotide sequence of any one of any one of the antisense strands set forth in any one of SEQ ID NOS: 4-1576 (e.g., any one of SEQ ID NOS: 4-19 or 772-787); and the nucleotide sequence of the sense strand comprises or consists of the nucleotide sequence of any one of the sense strands set forth in any one of SEQ ID NOS: 1577-3166 (e.g., any one of SEQ ID NOS: 1577-1592 or 2346-2361).

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the corresponding sense strand (e.g., as set forth in one of SEQ ID NOS: 1577-3166). In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the corresponding sense strand (e.g., as set forth in one of SEQ ID NOS: 1577-3166). In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the corresponding sense strand (e.g., as set forth in one of SEQ ID NOS: 1577-3166). In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the corresponding sense strand (e.g., as set forth in one of SEQ ID NOS: 1577-3166). In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the corresponding sense strand (e.g., as set forth in one of SEQ ID NOS: 1577-3166).

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the corresponding sense strand (e.g., as set forth in one of SEQ ID NOS: 1577-3166). In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the corresponding sense strand (e.g., as set forth in one of SEQ ID NOS: 1577-3166). In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the corresponding sense strand (e.g., as set forth in one of SEQ ID NOS: 1577-3166). In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises from about 21-23 (e.g., 21-22, 22-23) contiguous nucleotides differing by no more than 3 (e.g., 0, 1, 2, or 3) nucleotides from the nucleotide sequence of the corresponding sense strand (e.g., as set forth in one of SEQ ID NOS: 1577-3166).

In some embodiments, the antisense strand comprises or consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises or consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the corresponding sense strand (e.g., as set forth in one of SEQ ID NOS: 1577-3166). In some embodiments, the antisense strand comprises or consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises or consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the corresponding sense strand (e.g., as set forth in one of SEQ ID NOS: 1577-3166). In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises or consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the corresponding sense strand (e.g., as set forth in one of SEQ ID NOS: 1577-3166). In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises or consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the corresponding sense strand (e.g., as set forth in one of SEQ ID NOS: 1577-3166). In some embodiments, the antisense strand comprises or consists of about 23, 24, 25, 26, 27, 28, 29, 30 (e.g., 23) contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises or consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the corresponding sense strand (e.g., as set forth in one of SEQ ID NOS: 1577-3166). In some embodiments, the antisense strand comprises or consists of about 23 contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises or consists of about 21 contiguous nucleotides differing by no more than 3 (e.g., 0, 1, 2, or 3) nucleotides from the nucleotide sequence of the corresponding sense strand (e.g., as set forth in one of SEQ ID NOS: 1577-3166). In some embodiments, the antisense strand comprises or consists of about 21 contiguous nucleotides differing by no more than 3 (e.g., 0, 1, 2, or 3) nucleotides from the nucleotide sequence of any one of the antisense strands set forth in any one of SEQ ID NOS: 4-1576 (e.g., any one of SEQ ID NOS: 4-19 or 772-787); and the sense strand comprises or consists of about 19 contiguous nucleotides differing by no more than 3 (e.g., 0, 1, 2, or 3) nucleotides from the nucleotide sequence of the corresponding sense strand (e.g., as set forth in one of SEQ ID NOS: 1577-3166).

In some embodiments, the nucleotide sequence of the antisense strand comprises or consists of the nucleotide sequence of any one of the antisense strands set forth in any one of SEQ ID NOS: 4-1576 (e.g., any one of SEQ ID NOS: 4-19 or 772-787) differing by no more than 3 (e.g., 0, 1, 2, or 3) nucleotides from the antisense strand set forth in any one of SEQ ID NOS: 4-1576 (e.g., any one of SEQ ID NOS: 4-19 or 772-787); and the nucleotide sequence of the sense strand comprises or consists of the nucleotide sequence of the corresponding sense strand (e.g., as set forth in one of SEQ ID NOS: 1577-3166) differing by no more than 3 (e.g., 0, 1, 2, or 3) nucleotides from the sense strand set forth in any one of SEQ ID NOS: 1577-3166 (e.g., any one of SEQ ID NOS: 1577-1592 or 2346-2361).

In some embodiments, the nucleotide sequence of the antisense strand comprises or consists of the nucleotide sequence of any one of any one of the antisense strands set forth in any one of SEQ ID NOS: 4-1576 (e.g., any one of SEQ ID NOS: 4-19 or 772-787); and the nucleotide sequence of the sense strand comprises or consists of the nucleotide sequence of the corresponding sense strand (e.g., as set forth in one of SEQ ID NOS: 1577-3166).

In some embodiments, the antisense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises at least 15 (e.g., 15, 16, 17, 18, 19, 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises at least 19 (e.g., 20, 21, 22, 23, 24, 25, 26, 27, 28, 29, 30 (e.g., 19, 20, 21, 22, 23 (e.g., 23))) contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises at least 19 (e.g., 20, 21, 22, 23, 24, 25, 26, 27, 28, 29, 30 (e.g., 19, 20, 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises at least 21 (e.g., 22, 23, 24, 25, 26, 27, 28, 29, 30 (e.g., 21, 22, 23 (e.g., 23))) contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises at least 23 (e.g., 24, 25, 26, 27, 28, 29, 30 (e.g., 23)) contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises at least 21 (e.g., 22, 23, 24, 25, 26, 27, 28, 29, 30 (e.g., 21, 22, 23 (e.g., 21))) contiguous nucleotides differing by no more than 3 (e.g., 0, 1, 2, or 3) nucleotides from the nucleotide sequence of the sense strand of the corresponding (the same) dsRNA agent.

In some embodiments, the antisense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises from about 15-23 (e.g., 15-22, 15-21, 15-20, 15-19, 15-18, 15-17, 15-16, 16-22, 16-20, 16-19, 16-18, 16-17, 17-23, 17-22, 17-21, 17-20, 17-19, 17-18, 18-23, 18-22, 18-21, 18-20, 18-19, 19-23, 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises from about from about 21-23 (e.g., 21-22, 22-23) contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises from about 19-23 (e.g., 19-22, 19-21, 19-20, 20-23, 20-22, 20-21, 21-23, 21-22, 22-23) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises from about 21-23 (e.g., 21-22, 22-23) contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises from about 21-23 (e.g., 21-22, 22-23) contiguous nucleotides differing by no more than 3 (e.g., 0, 1, 2, or 3) nucleotides from the nucleotide sequence of the sense strand of the corresponding (the same) dsRNA agent.

In some embodiments, the antisense strand comprises or consists of about 15, 16, 17, 18, 19, 20, 21, 22, 23, 24, 25, 26, 27, 28, 29, or 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises or consists of about 15, 16, 17, 18, 19, 20, 21, 22, 23, 24, 25, 26, 27, 28, 29, or 30 (e.g., 19, 20, 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 19, 20, 21, 22, 23, 24, 25, 26, 27, 28, 29, 30 (e.g., 19, 20, 21, 22, 23 (e.g., 23)) contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises or consists of 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises or consists of about 19, 20, 21, 22, 23, 24, 25, 26, 27, 28, 29, 30 (e.g., 19, 20, 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 21, 22, 23, 24, 25, 26, 27, 28, 29, 30 (e.g., 21, 22, 23 (e.g., 23)) contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises or consists of at about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 23, 24, 25, 26, 27, 28, 29, 30 (e.g., 23) contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises or consists of about 21, 22, 23, 24, 25, 26, 27, 28, 29, 30 (e.g., 21, 22, 23 (e.g., 21)) contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 23 contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises or consists of about 21 contiguous nucleotides differing by no more than 3 (e.g., 0, 1, 2, or 3) nucleotides from the nucleotide sequence of the sense strand of the corresponding (the same) dsRNA agent. In some embodiments, the antisense strand comprises or consists of about 21 contiguous nucleotides differing by no more than 3 (e.g., 0, 1, 2, or 3) nucleotides from the nucleotide sequence of the antisense strand of any one of dsRNA agents 1-1590 (e.g., one of dsRNA agents 1-16 or 770-785); and the sense strand comprises or consists of about 19 contiguous nucleotides differing by no more than 3 (e.g., 0, 1, 2, or 3) nucleotides from the nucleotide sequence of the sense strand of the corresponding (the same) dsRNA agent.

In some embodiments, the nucleotide sequence of the antisense strand comprises or consists of the nucleotide sequence of the antisense strand of any one of dsRNA agents 1-1590 (e.g., one of dsRNA agents 1-16 or 770-785) differing by no more than 3 (e.g., 0, 1, 2, or 3) nucleotides from the antisense strand set forth in any one of the dsRNA agents 1-1590 (e.g., one of dsRNA agents 1-16 or 770-785); and the nucleotide sequence of the sense strand comprises or consists of the nucleotide sequence of the sense strand of the corresponding (the same) dsRNA agent differing by no more than 3 (e.g., 0, 1, 2, or 3) nucleotides from the sense strand set forth in the corresponding (the same) dsRNA agent.

In some embodiments, the nucleotide sequence of the antisense strand comprises or consists of the nucleotide sequence the antisense strand of any one of dsRNA agents 1-1590 (e.g., one of dsRNA agents 1-16 or 770-785); and the nucleotide sequence of the sense strand comprises or consists of the nucleotide sequence of the sense strand of the corresponding (the same) dsRNA agent.

4.2.3.6 Exemplary dsRNA Agents

The nucleotide sequence of exemplary unmodified dsRNA agents comprising a sense and antisense strand (e.g., suitable for targeting hBCL11A, suitable for inhibiting hBCL11A expression)) are set forth in Table 2. More specifically, Table 2 sets forth the nucleotide sequence of exemplary sense strands, antisense strands, and dsRNA agent pairs of sense and antisense strands. It is to be understood that while the sense and antisense strands are set forth in pairs in Table 2, the disclosure encompasses dsRNA agents comprising any sense strand and any antisense set forth in Table 2 (e.g., that are at least partially complementary (e.g., as could be determined by a person of ordinary skill in the art)). It is to be understood that while the nucleotide sequence of the sense strands and antisense strands in Table 2 are set forth as unmodified (not containing any modified nucleotides), the disclosure encompasses the sense and antisense sense strands set forth in Table 2 comprising one or more modified nucleotide (e.g., as described herein).

TABLE 2
Unmodified Sense and Antisense Strand Sequences of BCL11A dsRNA Agents.

| | | | | | | | Exemplary | |
|---|---|---|---|---|---|---|---|---|
| dsRNA | Sense | SEQ | Range in | Antisense | SEQ | Range in | mRNA Target | SEQ |
| Agent | Sequence | ID | NM_ | Sequence | ID | NM_ | Sequence in | ID |
| ID | 5′to 3′ | NO | 022893.4 | 5′to 3′ | NO | 022893.4 | NM_022893.4 | NO |
| 1 | UGGAUUAAG | 1577 | 963-983 | UCUAAGUAGA | 4 | 961-983 | CAUGGAUUAA | 3548 |
| | AAUCUACUU | | | UUCUUAAUCC | | | GAAUCUACUU | |
| | AGA | | | AUG | | | AGA | |
| 2 | CACAAACGG | 1578 | 580-600 | AUUGCAUUGU | 5 | 578-600 | AGCACAAACG | 3549 |
| | AAACAAUGC | | | UUCCGUUUGU | | | GAAACAAUGC | |
| | AAU | | | GCU | | | AAU | |
| 3 | ACAUUCUUA | 1579 | 560-580 | UCUCGAUAAA | 6 | 558-580 | GGACAUUCUU | 3550 |
| | UUUUUAUCG | | | AAUAAGAAUG | | | AUUUUUAUCG | |
| | AGA | | | UCC | | | AGC | |
| 4 | GCAAACAGC | 1580 | 908-928 | AACUGGUGAA | 7 | 906-928 | UUGCAAACAG | 3551 |
| | CAUUCACCA | | | UGGCUGUUUG | | | CCAUUCACCA | |
| | GUU | | | CAA | | | GUG | |
| 5 | AUGUACAAC | 1581 | 897-917 | UGCUGUUUGC | 8 | 895-917 | ACAUGUACAA | 3552 |
| | UUGCAAACA | | | AAGUUGUACA | | | CUUGCAAACA | |
| | GCA | | | UGU | | | GCC | |
| 6 | AACACGCAC | 1582 | 944-964 | AAUGAGUGUU | 9 | 942-964 | GCAACACGCA | 3553 |
| | AGAACACUC | | | CUGUGCGUGU | | | CAGAACACUC | |
| | AUU | | | UGC | | | AUG | |
| 7 | GCUACACAU | 1583 | 890-910 | UGCAAGUUGU | 10 | 888-910 | CAGCUACACA | 3554 |
| | GUACAACUU | | | ACAUGUGUAG | | | UGUACAACUU | |
| | GCA | | | CUG | | | GCA | |
| 8 | CACAUGUAC | 1584 | 894-914 | UGUUUGCAAG | 11 | 892-914 | UACACAUGUA | 3555 |
| | AACUUGCAA | | | UUGUACAUGU | | | CAACUUGCAA | |
| | ACA | | | GUA | | | ACA | |
| 9 | ACUUGCAAA | 1585 | 904-924 | UGUGAAUGGC | 12 | 902-924 | CAACUUGCAA | 3556 |
| | CAGCCAUUC | | | UGUUUGCAAG | | | ACAGCCAUUC | |
| | ACA | | | UUG | | | ACC | |
| 10 | CGCACAGAA | 1586 | 948-968 | UAUCCAUGAG | 13 | 946-968 | CACGCACAGA | 3557 |
| | CACUCAUGG | | | UGUUCUGUGC | | | ACACUCAUGG | |
| | AUA | | | GUG | | | AUU | |
| 11 | AUUCUUAUU | 1587 | 562-582 | UUGCUCGAUA | 14 | 560-582 | ACAUUCUUAU | 3558 |
| | UUUAUCGAG | | | AAAAUAAGAA | | | UUUUAUCGAG | |
| | CAA | | | UGU | | | CAC | |
| 12 | AGUGCAUGG | 1588 | 925-945 | UUGCAAGAGA | 15 | 923-945 | CCAGUGCAUG | 3559 |
| | UUUCUCUUG | | | AACCAUGCAC | | | GUUUCUCUUG | |
| | CAA | | | UGG | | | CAA | |
| 13 | UCAUCUAGA | 1589 | 730-750 | UGGGCAAAUU | 16 | 728-750 | CGUCAUCUAG | 3560 |
| | GGAAUUUGC | | | CCUCUAGAUG | | | AGGAAUUUGC | |
| | CCA | | | ACG | | | CCC | |
| 14 | GGCGAGAAG | 1590 | 2083-2103 | UCCGCGCUUA | 17 | 2081-2103 | UGGGCGAGAA | 3561 |
| | CAUAAGCGC | | | UGCUUCUCGC | | | GCAUAAGCGC | |
| | GGA | | | CCA | | | GGC | |
| 15 | GUAAGAUGC | 1591 | 2798-2818 | UCACGCUAAA | 18 | 2796-2818 | UUGUAAGAUG | 3562 |
| | CUUUUAGCG | | | AGGCAUCUUA | | | CCUUUUAGCG | |
| | UGA | | | CAA | | | UGU | |
| 16 | GGGAUUCAU | 1592 | 1069-1089 | AUUGUCUGCA | 19 | 1067-1089 | AUGGGAUUCA | 3563 |
| | AUUGCAGAC | | | AUAUGAAUCC | | | UAUUGCAGAC | |
| | AAU | | | CAU | | | AAU | |
| 17 | AUUAAGAAU | 1593 | 966-986 | CUUUCUAAGU | 20 | 964-986 | GGAUUAAGAA | 3564 |
| | CUACUUAGA | | | AGAUUCUUAA | | | UCUACUUAGA | |
| | AAG | | | UCC | | | AAG | |
| 18 | GAAGGACGU | 1594 | 2772-2792 | UCACAUUUGU | 21 | 2770-2792 | GGGAAGGACG | 3565 |
| | UUACAAAUG | | | AAACGUCCUU | | | UUUACAAAUG | |
| | UGA | | | CCC | | | UGA | |
| 19 | GUGAAAUUU | 1595 | 2789-2809 | AAGGCAUCUU | 22 | 2787- | AUGUGAAAUU | 3566 |
| | GUAAGAUGC | | | ACAAAUUUCA | | 2809 | UGUAAGAUGC | |
| | CUU | | | CAU | | | CUU | |
| 20 | ACGUUCAAA | 1596 | 1537-1557 | GUUGCUCUGA | 23 | 1535- | AGACGUUCAA | 3567 |
| | UUUCAGAGC | | | AAUUUGAACG | | 1557 | AUUUCAGAGC | |
| | AAC | | | UCU | | | AAC | |
| 21 | CACUCAUGG | 1597 | 957-977 | UAGAUUCUUA | 24 | 955-977 | AACACUCAUG | 3568 |
| | AUUAAGAAU | | | AUCCAUGAGU | | | GAUUAAGAAU | |
| | CUA | | | GUU | | | CUA | |
| 22 | CAACGUCAU | 1598 | 725-745 | AAAUUCCUCU | 25 | 723-745 | AUCAACGUCA | 3569 |
| | CUAGAGGAA | | | AGAUGACGUU | | | UCUAGAGGAA | |
| | UUU | | | GAU | | | UUU | |
| 23 | CUCUAGGAG | 1599 | 1329-1349 | AGCUCUCUAA | 26 | 1327-1349 | UUCUCUAGGA | 3570 |
| | ACUUAGAGA | | | GUCUCCUAGA | | | GACUUAGAGA | |
| | GCU | | | GAA | | | GCU | |
| 24 | ACAGAACAC | 1600 | 951-971 | CUUAAUCCAU | 27 | 949-971 | GCACAGAACA | 3571 |
| | UCAUGGAUU | | | GAGUGUUCUG | | | CUCAUGGAUU | |
| | AAG | | | UGC | | | AAG | |
| 25 | UGACAGGGU | 1601 | 1275-1295 | UUCAACCGCA | 28 | 1273-1295 | UUUGACAGGG | 3572 |
| | GCUGCGGUU | | | GCACCCUGUC | | | UGCUGCGGUU | |
| | GAA | | | AAA | | | GAA | |
| 26 | UCGAGUGUU | 1602 | 2859-2879 | AUAUCAUUAU | 29 | 2857-2879 | GAUCGAGUGU | 3573 |
| | GAAUAAUGA | | | UCAACACUCG | | | UGAAUAAUGA | |
| | UAU | | | AUC | | | UAU | |
| 27 | AUGACGAUU | 1603 | 710-730 | ACGUUGAUAA | 30 | 708-730 | GGAUGACGAU | 3574 |
| | GUUUAUCAA | | | ACAAUCGUCA | | | UGUUUAUCAA | |
| | CGU | | | UCC | | | CGU | |
| 28 | AUGGAUUUC | 1604 | 1321-1341 | AAGUCUCCUA | 31 | 1319-1341 | CCAUGGAUUU | 3575 |
| | UCUAGGAGA | | | GAGAAAUCCA | | | CUCUAGGAGA | |
| | CUU | | | UGG | | | CUU | |
| 29 | CUCCAGGCA | 1605 | 2379-2399 | GGAUCUUUGA | 32 | 2377-2399 | GCCUCCAGGC | 3576 |
| | GCUCAAAGA | | | GCUGCCUGGA | | | AGCUCAAAGA | |
| | UCC | | | GGC | | | UCC | |
| 30 | CUGUAGCAA | 1606 | 2643- | UGGACAGUGA | 33 | 2641-2663 | AACUGUAGCA | 3577 |
| | UCUCACUGU | | 2663 | GAUUGCUACA | | | AUCUCACUGU | |
| | CCA | | | GUU | | | CCA | |
| 31 | CGAGCACAA | 1607 | 576-596 | CAUUGUUUCC | 34 | 574-596 | AUCGAGCACA | 3578 |
| | ACGGAAACA | | | GUUUGUGCUC | | | AACGGAAACA | |
| | AUG | | | GAU | | | AUG | |
| 32 | UCAACGUCA | 1608 | 724-744 | AAUUCCUCUA | 35 | 722-744 | UAUCAACGUC | 3579 |
| | UCUAGAGGA | | | GAUGACGUUG | | | AUCUAGAGGA | |
| | AUU | | | AUA | | | AUU | |
| 33 | UGCGGUUGA | 1609 | 1286-1306 | UAGCCAUUGG | 36 | 1284-1306 | GCUGCGGUUG | 3580 |
| | AUCCAAUGG | | | AUUCAACCGC | | | AAUCCAAUGG | |
| | CUA | | | AGC | | | CUA | |
| 34 | GACGAUUGU | 1610 | 712-732 | UGACGUUGAU | 37 | 710-732 | AUGACGAUUG | 3581 |
| | UUAUCAACG | | | AAACAAUCGU | | | UUUAUCAACG | |
| | UCA | | | CAU | | | UCA | |
| 35 | UGGAGUCUC | 1611 | 284-304 | GUUAGAAAGA | 38 | 282-304 | UCUGGAGUCU | 3582 |
| | CUUCUUUCU | | | AGGAGACUCC | | | CCUUCUUUCU | |
| | AAC | | | AGA | | | AAC | |
| 36 | UGUGAAAUU | 1612 | 2788-2808 | AGGCAUCUUA | 39 | 2786-2808 | AAUGUGAAAU | 3583 |
| | UGUAAGAUG | | | CAAAUUUCAC | | | UUGUAAGAUG | |
| | CCU | | | AUU | | | CCU | |
| 37 | ACGGCUCUC | 1613 | 191-211 | GAUGAAUUGU | 40 | 189-211 | UGACGGCUCU | 3584 |
| | CCACAAUUC | | | GGGAGAGCCG | | | CCCACAAUUC | |
| | AUC | | | UCA | | | AUC | |
| 38 | GGAAAGUCU | 1614 | 2627-2647 | UACAGUUCUU | 41 | 2625-2647 | UGGGAAAGUC | 3585 |
| | UCAAGAACU | | | GAAGACUUUC | | | UUCAAGAACU | |
| | GUA | | | CCA | | | GUA | |
| 39 | UCCAGGCAG | 1615 | 2380-2400 | GGGAUCUUUG | 42 | 2378-2400 | CCUCCAGGCA | 3586 |
| | CUCAAAGAU | | | AGCUGCCUGG | | | GCUCAAAGAU | |
| | CCC | | | AGG | | | CCC | |
| 40 | GGAUGACGA | 1616 | 708-728 | GUUGAUAAAC | 43 | 706-728 | GAGGAUGACG | 3587 |
| | UUGUUUAUC | | | AAUCGUCAUC | | | AUUGUUUAUC | |
| | AAC | | | CUC | | | AAC | |
| 41 | CUGCGGUUG | 1617 | 1285-1305 | AGCCAUUGGA | 44 | 1283-1305 | UGCUGCGGUU | 3588 |
| | AAUCCAAUG | | | UUCAACCGCA | | | GAAUCCAAUG | |
| | GCU | | | GCA | | | GCU | |
| 42 | UGACGAUUG | 1618 | 711-731 | GACGUUGAUA | 45 | 709-731 | GAUGACGAUU | 3589 |
| | UUUAUCAAC | | | AACAAUCGUC | | | GUUUAUCAAC | |
| | GUC | | | AUC | | | GUC | |
| 43 | AUCCAAUGG | 1619 | 1295-1315 | GAGGCUCCAU | 46 | 1293-1315 | GAAUCCAAUG | 3590 |
| | CUAUGGAGC | | | AGCCAUUGGA | | | GCUAUGGAGC | |
| | CUC | | | UUC | | | CUC | |
| 44 | GCCUUAUAA | 1620 | 2688-2708 | CACAGCUCGC | 47 | 2686-2708 | AGGCCUUAUA | 3591 |
| | AUGCGAGCU | | | AUUUAUAAGG | | | AAUGCGAGCU | |
| | GUG | | | CCU | | | GUG | |
| 45 | CACUUAAGC | 1621 | 415-435 | GAAUUCCCGU | 48 | 413-435 | AGCACUUAAG | 3592 |
| | AAACGGGAA | | | UUGCUUAAGU | | | CAAACGGGAA | |
| | UUC | | | GCU | | | UUC | |
| 46 | GGAUUAAGA | 1622 | 964-984 | UUCUAAGUAG | 49 | 962-984 | AUGGAUUAAG | 3593 |
| | AUCUACUUA | | | AUUCUUAAUC | | | AAUCUACUUA | |
| | GAA | | | CAU | | | GAA | |
| 47 | CAAUCUCAC | 1623 | 2649-2669 | CUCCUGUGGA | 50 | 2647-2669 | AGCAAUCUCA | 3594 |
| | UGUCCACAG | | | CAGUGAGAUU | | | CUGUCCACAG | |
| | GAG | | | GCU | | | GAG | |
| 48 | AACCCGGCU | 1624 | 302-322 | UCACAUCGGG | 51 | 300-322 | CUAACCCGGC | 3595 |
| | CUCCCGAUG | | | AGAGCCGGGU | | | UCUCCCGAUG | |
| | UGA | | | UAG | | | UGA | |
| 49 | ACACUCAUG | 1625 | 956-976 | AGAUUCUUAA | 52 | 954-976 | GAACACUCAU | 3596 |
| | GAUUAAGAA | | | UCCAUGAGUG | | | GGAUUAAGAA | |
| | UCU | | | UUC | | | UCU | |
| 50 | CAUGACGGC | 1626 | 187-207 | AAUUGUGGGA | 53 | 185-207 | GCCAUGACGG | 3597 |
| | UCUCCCACA | | | GAGCCGUCAU | | | CUCUCCCACA | |
| | AUU | | | GGC | | | AUU | |
| 51 | GGACGUUUA | 1627 | 2775-2795 | AUUUCACAUU | 54 | 2773-2795 | AAGGACGUUU | 3598 |
| | CAAAUGUGA | | | UGUAAACGUC | | | ACAAAUGUGA | |
| | AAU | | | CUU | | | AAU | |
| 52 | AUGACGGCU | 1628 | 188-208 | GAAUUGUGGG | 55 | 186-208 | CCAUGACGGC | 3599 |
| | CUCCCACAA | | | AGAGCCGUCA | | | UCUCCCACAA | |
| | UUC | | | UGG | | | UUC | |
| 53 | AGCACAAAC | 1629 | 578-598 | UGCAUUGUUU | 56 | 576-598 | CGAGCACAAA | 3600 |
| | GGAAACAAU | | | CCGUUUGUGC | | | CGGAAACAAU | |
| | GCA | | | UCG | | | GCA | |
| 54 | CUGUGCAAC | 1630 | 2704-2724 | GGCACAGGCA | 57 | 2702-2724 | AGCUGUGCAA | 3601 |
| | UAUGCCUGU | | | UAGUUGCACA | | | CUAUGCCUGU | |
| | GCC | | | GCU | | | GCC | |
| 55 | UCGCCGCAA | 1631 | 390-410 | GGUUUGCCUU | 58 | 388-410 | UCUCGCCGCA | 3602 |
| | GCAAGGCAA | | | GCUUGCGGCG | | | AGCAAGGCAA | |
| | ACC | | | AGA | | | ACC | |
| 56 | UCUUGCAAC | 1632 | 938-958 | UGUUCUGUGC | 59 | 936-958 | UCUCUUGCAA | 3603 |
| | ACGCACAGA | | | GUGUUGCAAG | | | CACGCACAGA | |
| | ACA | | | AGA | | | ACA | |
| 57 | ACGUCAUCU | 1633 | 727-747 | GCAAAUUCCU | 60 | 725-747 | CAACGUCAUC | 3604 |
| | AGAGGAAUU | | | CUAGAUGACG | | | UAGAGGAAUU | |
| | UGC | | | UUG | | | UGC | |
| 58 | UCUGGAGUC | 1634 | 282-302 | UAGAAAGAAG | 61 | 280-302 | UCUCUGGAGU | 3605 |
| | UCCUUCUUU | | | GAGACUCCAG | | | CUCCUUCUUU | |
| | CUA | | | AGA | | | CUA | |
| 59 | UAACCCGGC | 1635 | 301-321 | CACAUCGGGA | 62 | 299-321 | UCUAACCCGG | 3606 |
| | UCUCCCGAU | | | GAGCCGGGUU | | | CUCUCCCGAU | |
| | GUG | | | AGA | | | GUG | |
| 60 | AGCACUUAA | 1636 | 413-433 | AUUCCCGUUU | 63 | 411-433 | CCAGCACUUA | 3607 |
| | GCAAACGGG | | | GCUUAAGUGC | | | AGCAAACGGG | |
| | AAU | | | UGG | | | AAU | |
| 61 | AAACGGAAA | 1637 | 583-603 | GCCAUUGCAU | 64 | 581-603 | ACAAACGGAA | 3608 |
| | CAAUGCAAU | | | UGUUUCCGUU | | | ACAAUGCAAU | |
| | GGC | | | UGU | | | GGC | |
| 62 | AUGGAUUAA | 1638 | 962-982 | CUAAGUAGAU | 65 | 960-982 | UCAUGGAUUA | 3609 |
| | GAAUCUACU | | | UCUUAAUCCA | | | AGAAUCUACU | |
| | UAG | | | UGA | | | UAG | |
| 63 | ACGUCCGCA | 1639 | 47-67 | UGCAAGUUCA | 66 | 45-67 | UCACGUCCGC | 3610 |
| | CUUGAACUU | | | AGUGCGGACG | | | ACUUGAACUU | |
| | GCA | | | UGA | | | GCA | |
| 64 | CACAUAGCA | 1640 | 760-780 | CAGAAGUUUA | 67 | 758-780 | AACACAUAGC | 3611 |
| | GAUAAACUU | | | UCUGCUAUGU | | | AGAUAAACUU | |
| | CUG | | | GUU | | | CUG | |
| 65 | UGCUCAGCU | 1641 | 2036-2056 | AGUGCUGCAU | 68 | 2034-2056 | GGUGCUCAGC | 3612 |
| | CCAUGCAGC | | | GGAGCUGAGC | | | UCCAUGCAGC | |
| | ACU | | | ACC | | | ACU | |
| 66 | ACAUAGCAG | 1642 | 761-781 | GCAGAAGUUU | 69 | 759-781 | ACACAUAGCA | 3613 |
| | AUAAACUUC | | | AUCUGCUAUG | | | GAUAAACUUC | |
| | UGC | | | UGU | | | UGC | |
| 67 | AACACGCAC | 1643 | 944-964 | CAUGAGUGUU | 70 | 942-964 | GCAACACGCA | 3553 |
| | AGAACACUC | | | CUGUGCGUGU | | | CAGAACACUC | |
| | AUG | | | UGC | | | AUG | |
| 68 | GCUGCGGUU | 1644 | 1284-1304 | GCCAUUGGAU | 71 | 1282-1304 | GUGCUGCGGU | 3554 |
| | GAAUCCAAU | | | UCAACCGCAG | | | UGAAUCCAAU | |
| | GGC | | | CAC | | | GGC | |
| 69 | UCCGCACUU | 1645 | 50-70 | AGCUGCAAGU | 72 | 48-70 | CGUCCGCACU | 3555 |
| | GAACUUGCA | | | UCAAGUGCGG | | | UGAACUUGCA | |
| | GCU | | | ACG | | | GCU | |
| 70 | GUUGAAUCC | 1646 | 1290-1310 | UCCAUAGCCA | 73 | 1288-1310 | CGGUUGAAUC | 3556 |
| | AAUGGCUAU | | | UUGGAUUCAA | | | CAAUGGCUAU | |
| | GGA | | | CCG | | | GGA | |
| 71 | AACUGUAGC | 1647 | 2641-2661 | GACAGUGAGA | 74 | 2639-2661 | AGAACUGUAG | 3557 |
| | AAUCUCACU | | | UUGCUACAGU | | | CAAUCUCACU | |
| | GUC | | | UCU | | | GUC | |
| 72 | CCGGCUCUC | 1648 | 305-325 | GGUUCACAUC | 75 | 303-325 | ACCCGGCUCU | 3558 |
| | CCGAUGUGA | | | GGGAGAGCCG | | | CCCGAUGUGA | |
| | ACC | | | GGU | | | ACC | |
| 73 | CUUCCCUGC | 1649 | 211-231 | UACAAAGAUG | 76 | 209-231 | AUCUUCCCUG | 3559 |
| | GCCAUCUUU | | | GCGCAGGGAA | | | CGCCAUCUUU | |
| | GUA | | | GAU | | | GUA | |
| 74 | AAUGGCAGC | 1650 | 598-618 | UUCUAAGCAG | 77 | 596-618 | GCAAUGGCAG | 3560 |
| | CUCUGCUUA | | | AGGCUGCCAU | | | CCUCUGCUUA | |
| | GAA | | | UGC | | | GAA | |
| 75 | CAAGACGUU | 1651 | 1533-1553 | CUCUGAAAUU | 78 | 1531-1553 | GGCAAGACGU | 3561 |
| | CAAAUUUCA | | | UGAACGUCUU | | | UCAAAUUUCA | |
| | GAG | | | GCC | | | GAG | |
| 76 | CCCGGGCAG | 1652 | 2571-2591 | UUUGAGCUGG | 79 | 2569-2591 | GGCCCGGGCA | 3562 |
| | GCCCAGCUC | | | GCCUGCCCGG | | | GGCCCAGCUC | |
| | AAA | | | GCC | | | AAA | |
| 77 | CGAGCUGUG | 1653 | 2700-2720 | CAGGCAUAGU | 80 | 2698-2720 | UGCGAGCUGU | 3563 |
| | CAACUAUGC | | | UGCACAGCUC | | | GCAACUAUGC | |
| | CUG | | | GCA | | | CUG | |
| 78 | GACAGGGUG | 1654 | 1276-1296 | AUUCAACCGC | 81 | 1274-1296 | UUGACAGGGU | 3564 |
| | CUGCGGUUG | | | AGCACCCUGU | | | GCUGCGGUUG | |
| | AAU | | | CAA | | | AAU | |
| 79 | GAAUCCAAU | 1655 | 1293-1313 | GGCUCCAUAG | 82 | 1291-1313 | UUGAAUCCAA | 3565 |
| | GGCUAUGGA | | | CCAUUGGAUU | | | UGGCUAUGGA | |
| | GCC | | | CAA | | | GCC | |
| 80 | UGCAACUAU | 1656 | 2707-2727 | CUGGGCACAG | 83 | 2705-2727 | UGUGCAACUA | 3566 |
| | GCCUGUGCC | | | GCAUAGUUGC | | | UGCCUGUGCC | |
| | CAG | | | ACA | | | CAG | |
| 81 | AGGCCUUAU | 1657 | 2686-2706 | CAGCUCGCAU | 84 | 2684-2706 | AAAGGCCUUA | 3567 |
| | AAAUGCGAG | | | UUAUAAGGCC | | | UAAAUGCGAG | |
| | CUG | | | UUU | | | CUG | |
| 82 | AUCACCCGA | 1658 | 1259-1279 | UGUCAAAGGC | 85 | 1257-1279 | CCAUCACCCG | 3568 |
| | GUGCCUUUG | | | ACUCGGGUGA | | | AGUGCCUUUG | |
| | ACA | | | UGG | | | ACA | |
| 83 | CCUGGCCAC | 1659 | 1248-1268 | CUCGGGUGAU | 86 | 1246-1268 | GCCCUGGCCA | 3569 |
| | CCAUCACCC | | | GGGUGGCCAG | | | CCCAUCACCC | |
| | GAG | | | GGC | | | GAG | |
| 84 | AAUUCAUCU | 1660 | 204-224 | AUGGCGCAGG | 87 | 202-224 | ACAAUUCAUC | 3570 |
| | UCCCUGCGC | | | GAAGAUGAAU | | | UUCCCUGCGC | |
| | CAU | | | UGU | | | CAU | |
| 85 | CCCUGCGCC | 1661 | 214-234 | UAAUACAAAG | 88 | 212-234 | UUCCCUGCGC | 3571 |
| | AUCUUUGUA | | | AUGGCGCAGG | | | CAUCUUUGUA | |
| | UUA | | | GAA | | | UUA | |
| 86 | CCACCCAUC | 1662 | 1253-1273 | AGGCACUCGG | 89 | 1251-1273 | GGCCACCCAU | 3572 |
| | ACCCGAGUG | | | GUGAUGGGUG | | | CACCCGAGUG | |
| | CCU | | | GCC | | | CCU | |
| 87 | UGGUUUCUC | 1663 | 931-951 | UGCGUGUUGC | 90 | 929-951 | CAUGGUUUCU | 3573 |
| | UUGCAACAC | | | AAGAGAAACC | | | CUUGCAACAC | |
| | GCA | | | AUG | | | GCA | |
| 88 | GGCCUCCAG | 1664 | 2376-2396 | UCUUUGAGCU | 91 | 2374-2396 | GCGGCCUCCA | 3574 |
| | GCAGCUCAA | | | GCCUGGAGGC | | | GGCAGCUCAA | |
| | AGA | | | CGC | | | AGA | |
| 89 | CAGCCAUUC | 1665 | 913-933 | CCAUGCACUG | 92 | 911-933 | AACAGCCAUU | 3575 |
| | ACCAGUGCA | | | GUGAAUGGCU | | | CACCAGUGCA | |
| | UGG | | | GUU | | | UGG | |
| 90 | GUCUCGCCG | 1666 | 387-407 | UUGCCUUGCU | 93 | 385-407 | AUGUCUCGCC | 3576 |
| | CAAGCAAGG | | | UGCGGCGAGA | | | GCAAGCAAGG | |
| | CAA | | | CAU | | | CAA | |
| 91 | CUUUGACAG | 1667 | 1272-1292 | AACCGCAGCA | 94 | 1270-1292 | GCCUUUGACA | 3577 |
| | GGUGCUGCG | | | CCCUGUCAAA | | | GGGUGCUGCG | |
| | GUU | | | GGC | | | GUU | |
| 92 | UUCAUCUUC | 1668 | 206-226 | AGAUGGCGCA | 95 | 204-226 | AAUUCAUCUU | 3578 |
| | CCUGCGCCA | | | GGGAAGAUGA | | | CCCUGCGCCA | |
| | UCU | | | AUU | | | UCU | |
| 93 | AAAGAUGAG | 1669 | 874-894 | GUAGCUGCUG | 96 | 872-894 | GUAAAGAUGA | 3579 |
| | CCCAGCAGC | | | GGCUCAUCUU | | | GCCCAGCAGC | |
| | UAC | | | UAC | | | UAC | |
| 94 | GAGUGCCUU | 1670 | 1266-1286 | AGCACCCUGU | 97 | 1264-1286 | CCGAGUGCCU | 3580 |
| | UGACAGGGU | | | CAAAGGCACU | | | UUGACAGGGU | |
| | GCU | | | CGG | | | GCU | |
| 95 | AGAGGGCAG | 1671 | 2592-2612 | GUGUCGCUGC | 98 | 2590-2612 | AAAGAGGGCA | 3581 |
| | ACGCAGCGA | | | GUCUGCCCUC | | | GACGCAGCGA | |
| | CAC | | | UUU | | | CAC | |
| 96 | AUCUUCCCU | 1672 | 209-229 | CAAAGAUGGC | 99 | 207-229 | UCAUCUUCCC | 3582 |
| | GCGCCAUCU | | | GCAGGGAAGA | | | UGCGCCAUCU | |
| | UUG | | | UGA | | | UUG | |
| 97 | AUUCUUACA | 1673 | 457-477 | UGGUUCAUCA | 100 | 455-477 | CCAUUCUUAC | 3583 |
| | GAUGAUGAA | | | UCUGUAAGAA | | | AGAUGAUGAA | |
| | CCA | | | UGG | | | CCA | |
| 98 | GUCAAGUCC | 1674 | 1504-1524 | CUCGCAUGAC | 101 | 1502-1524 | CGGUCAAGUC | 3584 |
| | AAGUCAUGC | | | UUGGACUUGA | | | CAAGUCAUGC | |
| | GAG | | | CCG | | | GAG | |
| 99 | UCACCCGAG | 1675 | 1260-1280 | CUGUCAAAGG | 102 | 1258-1280 | CAUCACCCGA | 3585 |
| | UGCCUUUGA | | | CACUCGGGUG | | | GUGCCUUUGA | |
| | CAG | | | AUG | | | CAG | |
| 100 | CGUCAUCUA | 1676 | 728-748 | GGCAAAUUCC | 103 | 726-748 | AACGUCAUCU | 3586 |
| | GAGGAAUUU | | | UCUAGAUGAC | | | AGAGGAAUUU | |
| | GCC | | | GUU | | | GCC | |
| 101 | GUGCAACUA | 1677 | 2706-2726 | UGGGCACAGG | 104 | 2704-2726 | CUGUGCAACU | 3587 |
| | UGCCUGUGC | | | CAUAGUUGCA | | | AUGCCUGUGC | |
| | CCA | | | CAG | | | CCA | |
| 102 | GGCUCUCCC | 1678 | 193-213 | AAGAUGAAUU | 105 | 191-213 | ACGGCUCUCC | 3588 |
| | ACAAUUCAU | | | GUGGGAGAGC | | | CACAAUUCAU | |
| | CUU | | | CGU | | | CUU | |
| 103 | UAAGCAAAC | 1679 | 419-439 | GCGAGAAUUC | 106 | 417-439 | CUUAAGCAAA | 3589 |
| | GGGAAUUCU | | | CCGUUUGCUU | | | CGGGAAUUCU | |
| | CGC | | | AAG | | | CGC | |
| 104 | GGCCCUGGC | 1680 | 1245-1265 | GGGUGAUGGG | 107 | 1243-1265 | AUGGCCCUGG | 3590 |
| | CACCCAUCA | | | UGGCCAGGGC | | | CCACCCAUCA | |
| | CCC | | | CAU | | | CCC | |
| 105 | AGAAGCCAC | 1681 | 2668-2688 | CCUUUCGCCC | 108 | 2666-2688 | GGAGAAGCCA | 3591 |
| | ACGGGCGAA | | | GUGUGGCUUC | | | CACGGGCGAA | |
| | AGG | | | UCC | | | AGG | |
| 106 | CCCGGCUCU | 1682 | 304-324 | GUUCACAUCG | 109 | 302-324 | AACCCGGCUC | 3592 |
| | CCCGAUGUG | | | GGAGAGCCGG | | | UCCCGAUGUG | |
| | AAC | | | GUU | | | AAC | |
| 107 | GGAGAAGCC | 1683 | 2666-2686 | UUUCGCCCGU | 110 | 2664-2686 | CAGGAGAAGC | 3593 |
| | ACACGGGCG | | | GUGGCUUCUC | | | CACACGGGCG | |
| | AAA | | | CUG | | | AAA | |
| 108 | UGCAAUGGC | 1684 | 595-615 | UAAGCAGAGG | 111 | 593-615 | AAUGCAAUGG | 3594 |
| | AGCCUCUGC | | | CUGCCAUUGC | | | CAGCCUCUGC | |
| | UUA | | | AUU | | | UUA | |
| 109 | GCACUCCUC | 1685 | 2451-2471 | CUCCCGUUCU | 112 | 2449-2471 | GAGCACUCCU | 3595 |
| | GGAGAACGG | | | CCGAGGAGUG | | | CGGAGAACGG | |
| | GAG | | | CUC | | | GAG | |
| 110 | UUGUUUAUC | 1686 | 717-737 | CUAGAUGACG | 113 | 715-737 | GAUUGUUUAU | 3596 |
| | AACGUCAUC | | | UUGAUAAACA | | | CAACGUCAUC | |
| | UAG | | | AUC | | | UAG | |
| 111 | CAGGGUGCU | 1687 | 1278-1298 | GGAUUCAACC | 114 | 1276-1298 | GACAGGGUGC | 3597 |
| | GCGGUUGAA | | | GCAGCACCCU | | | UGCGGUUGAA | |
| | UCC | | | GUC | | | UCC | |
| 112 | UAUCGAGCA | 1688 | 573-593 | UGUUUCCGUU | 115 | 571-593 | UUUAUCGAGC | 3598 |
| | CAAACGGAA | | | UGUGCUCGAU | | | ACAAACGGAA | |
| | ACA | | | AAA | | | ACA | |
| 113 | CUCCCUCCU | 1689 | 1462-1482 | GGCGGAUUGC | 116 | 1460-1482 | CCCUCCCUCC | 3599 |
| | CUGCAAUCC | | | AGAGGAGGGA | | | UCUGCAAUCC | |
| | GCC | | | GGG | | | GCC | |
| 114 | GUAUCCCUU | 1690 | 1019-1039 | CACCUAGUCC | 117 | 1017-1039 | UGGUAUCCCU | 3600 |
| | CAGGACUAG | | | UGAAGGGAUA | | | UCAGGACUAG | |
| | GUG | | | CCA | | | GUG | |
| 115 | UCCUUCUUU | 1691 | 291-311 | GAGCCGGGUU | 118 | 289-311 | UCUCCUUCUU | 3601 |
| | CUAACCCGG | | | AGAAAGAAGG | | | UCUAACCCGG | |
| | CUC | | | AGA | | | CUC | |
| 116 | CCAUGACGG | 1692 | 186-206 | AUUGUGGGAG | 119 | 184-206 | AGCCAUGACG | 3602 |
| | CUCUCCCAC | | | AGCCGUCAUG | | | GCUCUCCCAC | |
| | AAU | | | GCU | | | AAU | |
| 117 | UUUGACAGG | 1693 | 1273-1293 | CAACCGCAGC | 120 | 1271-1293 | CCUUUGACAG | 3603 |
| | GUGCUGCGG | | | ACCCUGUCAA | | | GGUGCUGCGG | |
| | UUG | | | AGG | | | UUG | |
| 118 | GCGGGUUGG | 1694 | 1011-1031 | CCUGAAGGGA | 121 | 1009-1031 | CCGCGGGUUG | 3604 |
| | UAUCCCUUC | | | UACCAACCCG | | | GUAUCCCUUC | |
| | AGG | | | CGG | | | AGG | |
| 119 | UGUGCAACU | 1695 | 2705-2725 | GGGCACAGGC | 122 | 2703-2725 | GCUGUGCAAC | 3605 |
| | AUGCCUGUG | | | AUAGUUGCAC | | | UAUGCCUGUG | |
| | CCC | | | AGC | | | CCC | |
| 120 | GUGCUGCGG | 1696 | 1282-1302 | CAUUGGAUUC | 123 | 1280-1302 | GGGUGCUGCG | 3606 |
| | UUGAAUCCA | | | AACCGCAGCA | | | GUUGAAUCCA | |
| | AUG | | | CCC | | | AUG | |
| 121 | CAGCACUUA | 1697 | 412-432 | UUCCCGUUUG | 124 | 410-432 | CCCAGCACUU | 3607 |
| | AGCAAACGG | | | CUUAAGUGCU | | | AAGCAAACGG | |
| | GAA | | | GGG | | | GAA | |
| 122 | UUGAAUCCA | 1698 | 1291-1311 | CUCCAUAGCC | 125 | 1289-1311 | GGUUGAAUCC | 3608 |
| | AUGGCUAUG | | | AUUGGAUUCA | | | AAUGGCUAUG | |
| | GAG | | | ACC | | | GAG | |
| 123 | CUGGCAGAA | 1699 | 1144-1164 | UGGAAAGCGC | 126 | 1142-1164 | GCCUGGCAGA | 3609 |
| | GGGCGCUUU | | | CCUUCUGCCA | | | AGGGCGCUUU | |
| | CCA | | | GGC | | | CCA | |
| 124 | GGAUGAGUG | 1700 | 839-859 | GGGCAUAUUC | 127 | 837-859 | UGGGAUGAGU | 3610 |
| | CAGAAUAUG | | | UGCACUCAUC | | | GCAGAAUAUG | |
| | ccc | | | CCA | | | CCC | |
| 125 | UCUCCUUCU | 1701 | 289-309 | GCCGGGUUAG | 128 | 287-309 | AGUCUCCUUC | 3611 |
| | UUCUAACCC | | | AAAGAAGGAG | | | UUUCUAACCC | |
| | GGC | | | ACU | | | GGC | |
| 126 | UGCUGCGGU | 1702 | 1283- | CCAUUGGAUU | 129 | 1281-1303 | GGUGCUGCGG | 3612 |
| | UGAAUCCAA | | 1303 | CAACCGCAGC | | | UUGAAUCCAA | |
| | UGG | | | ACC | | | UGG | |
| 127 | GGAGUCUCC | 1703 | 285-305 | GGUUAGAAAG | 130 | 283-305 | CUGGAGUCUC | 3613 |
| | UUCUUUCUA | | | AAGGAGACUC | | | CUUCUUUCUA | |
| | ACC | | | CAG | | | ACC | |
| 128 | GCCCUGGCC | 1704 | 1246- | CGGGUGAUGG | 131 | 1244-1266 | UGGCCCUGGC | 3614 |
| | ACCCAUCAC | | 1266 | GUGGCCAGGG | | | CACCCAUCAC | |
| | CCG | | | CCA | | | CCG | |
| 129 | CGGCUCUCC | 1705 | 192-212 | AGAUGAAUUG | 132 | 190-212 | GACGGCUCUC | 3615 |
| | CACAAUUCA | | | UGGGAGAGCC | | | CCACAAUUCA | |
| | UCU | | | GUC | | | UCU | |
| 130 | GAGAAGCCA | 1706 | 2667- | CUUUCGCCCG | 133 | 2665-2687 | AGGAGAAGCC | 3616 |
| | CACGGGCGA | | 2687 | UGUGGCUUCU | | | ACACGGGCGA | |
| | AAG | | | CCU | | | AAG | |
| 131 | UGGCCCUGG | 1707 | 1244- | GGUGAUGGGU | 134 | 1242-1264 | GAUGGCCCUG | 3617 |
| | CCACCCAUC | | 1264 | GGCCAGGGCC | | | GCCACCCAUC | |
| | ACC | | | AUC | | | ACC | |
| 132 | AUAAACUUC | 1708 | 770-790 | CCCUCCAGUG | 135 | 768-790 | AGAUAAACUU | 3618 |
| | UGCACUGGA | | | CAGAAGUUUA | | | CUGCACUGGA | |
| | GGG | | | UCU | | | GGG | |
| 133 | CUAACCCGG | 1709 | 300-320 | ACAUCGGGAG | 136 | 298-320 | UUCUAACCCG | 3619 |
| | CUCUCCCGA | | | AGCCGGGUUA | | | GCUCUCCCGA | |
| | UGU | | | GAA | | | UGU | |
| 134 | AUUUCUCUA | 1710 | 1325- | CUCUAAGUCU | 137 | 1323-1345 | GGAUUUCUCU | 3620 |
| | GGAGACUUA | | 1345 | CCUAGAGAAA | | | AGGAGACUUA | |
| | GAG | | | UCC | | | GAG | |
| 135 | UUCUUUCUA | 1711 | 294-314 | GGAGAGCCGG | 138 | 292-314 | CCUUCUUUCU | 3621 |
| | ACCCGGCUC | | | GUUAGAAAGA | | | AACCCGGCUC | |
| | UCC | | | AGG | | | UCC | |
| 136 | UUGACAGGG | 1712 | 1274- | UCAACCGCAG | 139 | 1272-1294 | CUUUGACAGG | 3622 |
| | UGCUGCGGU | | 1294 | CACCCUGUCA | | | GUGCUGCGGU | |
| | UGA | | | AAG | | | UGA | |
| 137 | AGCACUCCU | 1713 | 2450- | UCCCGUUCUC | 140 | 2448-2470 | GGAGCACUCC | 3623 |
| | CGGAGAACG | | 2470 | CGAGGAGUGC | | | UCGGAGAACG | |
| | GGA | | | UCC | | | GGA | |
| 138 | CCCUGGCCA | 1714 | 1247- | UCGGGUGAUG | 141 | 1245-1267 | GGCCCUGGCC | 3624 |
| | CCCAUCACC | | 1267 | GGUGGCCAGG | | | ACCCAUCACC | |
| | CGA | | | GCC | | | CGA | |
| 139 | AGCAAACGG | 1715 | 421-441 | GGGCGAGAAU | 142 | 419-441 | UAAGCAAACG | 3625 |
| | GAAUUCUCG | | | UCCCGUUUGC | | | GGAAUUCUCG | |
| | CCC | | | UUA | | | CCC | |
| 140 | GCUGGCAGG | 1716 | 1347- | CUAGACGUGU | 143 | 1345-1367 | GAGCUGGCAG | 3626 |
| | GAACACGUC | | 1367 | UCCCUGCCAG | | | GGAACACGUC | |
| | UAG | | | CUC | | | UAG | |
| 141 | CCCUAUGCA | 1717 | 1395- | UGCAGUAACC | 144 | 1393-1415 | AGCCCUAUGC | 3627 |
| | AAGGUUACU | | 1415 | UUUGCAUAGG | | | AAAGGUUACU | |
| | GCA | | | GCU | | | GCA | |
| 142 | UCAUCUUCC | 1718 | 207-227 | AAGAUGGCGC | 145 | 205-227 | AUUCAUCUUC | 3628 |
| | CUGCGCCAU | | | AGGGAAGAUG | | | CCUGCGCCAU | |
| | CUU | | | AAU | | | CUU | |
| 143 | CUUCUUUCU | 1719 | 293-313 | GAGAGCCGGG | 146 | 291-313 | UCCUUCUUUC | 3629 |
| | AACCCGGCU | | | UUAGAAAGAA | | | UAACCCGGCU | |
| | CUC | | | GGA | | | CUC | |
| 144 | CCACCACCG | 1720 | 1186- | CAAGUGAUGU | 147 | 1184-1206 | GUCCACCACC | 3630 |
| | AGACAUCAC | | 1206 | CUCGGUGGUG | | | GAGACAUCAC | |
| | UUG | | | GAC | | | UUG | |
| 145 | AACCAUUCC | 1721 | 1415- | UGCUACCUGG | 148 | 1413-1435 | GCAACCAUUC | 3631 |
| | AGCCAGGUA | | 1435 | CUGGAAUGGU | | | CAGCCAGGUA | |
| | GCA | | | UGC | | | GCA | |
| 146 | UCUUCCCUG | 1722 | 210-230 | ACAAAGAUGG | 149 | 208-230 | CAUCUUCCCU | 3632 |
| | CGCCAUCUU | | | CGCAGGGAAG | | | GCGCCAUCUU | |
| | UGU | | | AUG | | | UGU | |
| 147 | CUGGCCACC | 1723 | 1249- | ACUCGGGUGA | 150 | 1247-1269 | CCCUGGCCAC | 3633 |
| | CAUCACCCG | | 1269 | UGGGUGGCCA | | | CCAUCACCCG | |
| | AGU | | | GGG | | | AGU | |
| 148 | GAGGGCAGA | 1724 | 2593-2613 | AGUGUCGCUG | 151 | 2591- | AAGAGGGCAG | 3634 |
| | CGCAGCGAC | | | CGUCUGCCCU | | 2613 | ACGCAGCGAC | |
| | ACU | | | CUU | | | ACU | |
| 149 | CACAAUUCA | 1725 | 201-221 | GCGCAGGGAA | 152 | 199-221 | CCCACAAUUC | 3635 |
| | UCUUCCCUG | | | GAUGAAUUGU | | | AUCUUCCCUG | |
| | CGC | | | GGG | | | CGC | |
| 150 | CCAUCACCC | 1726 | 1257-1277 | UCAAAGGCAC | 153 | 1255-1277 | ACCCAUCACC | 3636 |
| | GAGUGCCUU | | | UCGGGUGAUG | | | CGAGUGCCUU | |
| | UGA | | | GGU | | | UGA | |
| 151 | ACACGCACA | 1727 | 945-965 | CCAUGAGUGU | 154 | 943-965 | CAACACGCAC | 3637 |
| | GAACACUCA | | | UCUGUGCGUG | | | AGAACACUCA | |
| | UGG | | | UUG | | | UGG | |
| 152 | GAAGCCACA | 1728 | 2669-2689 | GCCUUUCGCC | 155 | 2667-2689 | GAGAAGCCAC | 3638 |
| | CGGGCGAAA | | | CGUGUGGCUU | | | ACGGGCGAAA | |
| | GGC | | | CUC | | | GGC | |
| 153 | CAUCACCCG | 1729 | 1258-1278 | GUCAAAGGCA | 156 | 1256-1278 | CCCAUCACCC | 3639 |
| | AGUGCCUUU | | | CUCGGGUGAU | | | GAGUGCCUUU | |
| | GAC | | | GGG | | | GAC | |
| 154 | UCAAGUUCG | 1730 | 28-48 | GUGACGUCCC | 157 | 26-48 | GUUCAAGUUC | 3640 |
| | CAGGGACGU | | | UGCGAACUUG | | | GCAGGGACGU | |
| | CAC | | | AAC | | | CAC | |
| 155 | UCUAACCCG | 1731 | 299-319 | CAUCGGGAGA | 158 | 297-319 | UUUCUAACCC | 3641 |
| | GCUCUCCCG | | | GCCGGGUUAG | | | GGCUCUCCCG | |
| | AUG | | | AAA | | | AUG | |
| 156 | GGGAAGUGG | 1732 | 2532-2552 | GGCGUGCUCC | 159 | 2530-2552 | ACGGGAAGUG | 3642 |
| | AGGGAGCAC | | | CUCCACUUCC | | | GAGGGAGCAC | |
| | GCC | | | CGU | | | GCC | |
| 157 | CUCGCCGCA | 1733 | 389-409 | GUUUGCCUUG | 160 | 387-409 | GUCUCGCCGC | 3643 |
| | AGCAAGGCA | | | CUUGCGGCGA | | | AAGCAAGGCA | |
| | AAC | | | GAC | | | AAC | |
| 158 | GAGCUGGCA | 1734 | 1345-1365 | AGACGUGUUC | 161 | 1343-1365 | GAGAGCUGGC | 3644 |
| | GGGAACACG | | | CCUGCCAGCU | | | AGGGAACACG | |
| | UCU | | | CUC | | | UCU | |
| 159 | ACCAGUGCA | 1735 | 922-942 | CAAGAGAAAC | 162 | 920-942 | UCACCAGUGC | 3645 |
| | UGGUUUCUC | | | CAUGCACUGG | | | AUGGUUUCUC | |
| | UUG | | | UGA | | | UUG | |
| 160 | GCAUCAAGC | 1736 | 2288-2308 | ACUCCUUCUC | 163 | 2286-2308 | GCGCAUCAAG | 3646 |
| | UCGAGAAGG | | | GAGCUUGAUG | | | CUCGAGAAGG | |
| | AGU | | | CGC | | | AGU | |
| 161 | CUUCCUUAG | 1737 | 2400-2420 | GAGUCUCCGA | 164 | 2398-2420 | CCCUUCCUUA | 3647 |
| | CUUCGGAGA | | | AGCUAAGGAA | | | GCUUCGGAGA | |
| | CUC | | | GGG | | | CUC | |
| 162 | GAGGAUGAC | 1738 | 706-726 | UGAUAAACAA | 165 | 704-726 | CAGAGGAUGA | 3648 |
| | GAUUGUUUA | | | UCGUCAUCCU | | | CGAUUGUUUA | |
| | UCA | | | CUG | | | UCA | |
| 163 | CAUCCAGAC | 1739 | 10-30 | UGAACGUCAG | 166 | 8-30 | UCCAUCCAGA | 3649 |
| | UCCUGACGU | | | GAGUCUGGAU | | | CUCCUGACGU | |
| | UCA | | | GGA | | | UCA | |
| 164 | CAGGGACAC | 1740 | 2127-2147 | UCUUCGUCGC | 167 | 2125-2147 | CACAGGGACA | 3650 |
| | UUGCGACGA | | | AAGUGUCCCU | | | CUUGCGACGA | |
| | AGA | | | GUG | | | AGA | |
| 165 | ACCAAAGGU | 1741 | 5939-5959 | CAGGUUGUAA | 168 | 5937-5959 | UGACCAAAGG | 3651 |
| | CAUUACAAC | | | UGACCUUUGG | | | UCAUUACAAC | |
| | CUG | | | UCA | | | CUG | |
| 166 | CGUUCAAAU | 1742 | 1538-1558 | GGUUGCUCUG | 169 | 1536-1558 | GACGUUCAAA | 3652 |
| | UUCAGAGCA | | | AAAUUUGAAC | | | UUUCAGAGCA | |
| | ACC | | | GUC | | | ACC | |
| 167 | CAGCCCUAU | 1743 | 1392-1412 | AGUAACCUUU | 170 | 1390-1412 | CCCAGCCCUA | 3653 |
| | GCAAAGGUU | | | GCAUAGGGCU | | | UGCAAAGGUU | |
| | ACU | | | GGG | | | ACU | |
| 168 | CACAGGGAC | 1744 | 2125-2145 | UUCGUCGCAA | 171 | 2123-2145 | GCCACAGGGA | 3654 |
| | ACUUGCGAC | | | GUGUCCCUGU | | | CACUUGCGAC | |
| | GAA | | | GGC | | | GAA | |
| 169 | GCAUCCAAU | 1745 | 667-687 | AACCUCCACG | 172 | 665-687 | AAGCAUCCAA | 3655 |
| | CCCGUGGAG | | | GGAUUGGAUG | | | UCCCGUGGAG | |
| | GUU | | | CUU | | | GUU | |
| 170 | GCCCAACAC | 1746 | 2331-2351 | UACACGUUCU | 173 | 2329-2351 | AUGCCCAACA | 3656 |
| | GGAGAACGU | | | CCGUGUUGGG | | | CGGAGAACGU | |
| | GUA | | | CAU | | | GUA | |
| 171 | CCAGCCACC | 1747 | 1053-1073 | AUCCCAUGGA | 174 | 1051-1073 | UCCCAGCCAC | 3657 |
| | UCUCCAUGG | | | GAGGUGGCUG | | | CUCUCCAUGG | |
| | GAU | | | GGA | | | GAU | |
| 172 | CCACCUCUC | 1748 | 1057-1077 | AUGAAUCCCA | 175 | 1055-1077 | AGCCACCUCU | 3658 |
| | CAUGGGAUU | | | UGGAGAGGUG | | | CCAUGGGAUU | |
| | CAU | | | GCU | | | CAU | |
| 173 | CGGCCCAGC | 1749 | 1387-1407 | CCUUUGCAUA | 176 | 1385-1407 | GCCGGCCCAG | 3659 |
| | CCUAUGCAA | | | GGGCUGGGCC | | | CCCUAUGCAA | |
| | AGG | | | GGC | | | AGG | |
| 174 | GGCACAGUG | 1750 | 2849-2869 | UCAACACUCG | 177 | 2847-2869 | AUGGCACAGU | 3660 |
| | AUCGAGUGU | | | AUCACUGUGC | | | GAUCGAGUGU | |
| | UGA | | | CAU | | | UGA | |
| 175 | CCUGGGCGA | 1751 | 2079-2099 | CGCUUAUGCU | 178 | 2077-2099 | GUCCUGGGCG | 3661 |
| | GAAGCAUAA | | | UCUCGCCCAG | | | AGAAGCAUAA | |
| | GCG | | | GAC | | | GCG | |
| 176 | UCCUGACGU | 1752 | 19-39 | CUGCGAACUU | 179 | 17-39 | ACUCCUGACG | 3662 |
| | UCAAGUUCG | | | GAACGUCAGG | | | UUCAAGUUCG | |
| | CAG | | | AGU | | | CAG | |
| 177 | GAUCGAGUG | 1753 | 2857-2877 | AUCAUUAUUC | 180 | 2855-2877 | GUGAUCGAGU | 3663 |
| | UUGAAUAAU | | | AACACUCGAU | | | GUUGAAUAAU | |
| | GAU | | | CAC | | | GAU | |
| 178 | ACGCCAGAG | 1754 | 700-720 | ACAAUCGUCA | 181 | 698-720 | UCACGCCAGA | 3664 |
| | GAUGACGAU | | | UCCUCUGGCG | | | GGAUGACGAU | |
| | UGU | | | UGA | | | UGU | |
| 179 | GGUUGGCAU | 1755 | 684-704 | GGCGUGACCU | 182 | 682-704 | GAGGUUGGCA | 3665 |
| | CCAGGUCAC | | | GGAUGCCAAC | | | UCCAGGUCAC | |
| | GCC | | | CUC | | | GCC | |
| 180 | GUGGCCAAG | 1756 | 1783-1803 | CUCGCUCUUG | 183 | 1781-1803 | UGGUGGCCAA | 3666 |
| | UUCAAGAGC | | | AACUUGGCCA | | | GUUCAAGAGC | |
| | GAG | | | CCA | | | GAG | |
| 181 | UACAAGUGC | 1757 | 1597-1617 | GUCGCACAGG | 184 | 1595-1617 | CCUACAAGUG | 3667 |
| | AACCUGUGC | | | UUGCACUUGU | | | CAACCUGUGC | |
| | GAC | | | AGG | | | GAC | |
| 182 | AGAUCCCUU | 1758 | 2394-2414 | CCGAAGCUAA | 185 | 2392-2414 | AAAGAUCCCU | 3668 |
| | CCUUAGCUU | | | GGAAGGGAUC | | | UCCUUAGCUU | |
| | CGG | | | UUU | | | CGG | |
| 183 | UCCAGACUC | 1759 | 12-32 | CUUGAACGUC | 186 | 10-32 | CAUCCAGACU | 3669 |
| | CUGACGUUC | | | AGGAGUCUGG | | | CCUGACGUUC | |
| | AAG | | | AUG | | | AAG | |
| 184 | GAGCUGGAC | 1760 | 2497-2517 | CGAGAUCCCU | 187 | 2495-2495 | GGGAGCUGGA | 3670 |
| | GGAGGGAUC | | | CCGUCCAGCU | | | CGGAGGGAUC | |
| | UCG | | | CCC | | | UCG | |
| 185 | ACAGGGACA | 1761 | 2126-2146 | CUUCGUCGCA | 188 | 2124-2146 | CCACAGGGAC | 3671 |
| | CUUGCGACG | | | AGUGUCCCUG | | | ACUUGCGACG | |
| | AAG | | | UGG | | | AAG | |
| 186 | CCUUCCUUA | 1762 | 2399-2419 | AGUCUCCGAA | 189 | 2397-2419 | UCCCUUCCUU | 3672 |
| | GCUUCGGAG | | | GCUAAGGAAG | | | AGCUUCGGAG | |
| | ACU | | | GGA | | | ACU | |
| 187 | AGGUCCUGG | 1763 | 2075-2095 | UAUGCUUCUC | 190 | 2073-2095 | CCAGGUCCUG | 3673 |
| | GCGAGAAGC | | | GCCCAGGACC | | | GGCGAGAAGC | |
| | AUA | | | UGG | | | AUA | |
| 188 | AUUUCAGAG | 1764 | 1545-1565 | ACCACCAGGU | 191 | 1543-1565 | AAAUUUCAGA | 3674 |
| | CAACCUGGU | | | UGCUCUGAAA | | | GCAACCUGGU | |
| | GGU | | | UUU | | | GGU | |
| 189 | AGAGGAUGA | 1765 | 705-725 | GAUAAACAAU | 192 | 703-725 | CCAGAGGAUG | 3675 |
| | CGAUUGUUU | | | CGUCAUCCUC | | | ACGAUUGUUU | |
| | AUC | | | UGG | | | AUC | |
| 190 | UGGUGGCCA | 1766 | 1781-1801 | CGCUCUUGAA | 193 | 1779-1801 | CGUGGUGGCC | 3676 |
| | AGUUCAAGA | | | CUUGGCCACC | | | AAGUUCAAGA | |
| | GCG | | | ACG | | | GCG | |
| 191 | GCCGGCCCA | 1767 | 1385-1405 | UUUGCAUAGG | 194 | 1383-1405 | AGGCCGGCCC | 3677 |
| | GCCCUAUGC | | | GCUGGGCCGG | | | AGCCCUAUGC | |
| | AAA | | | CCU | | | AAA | |
| 192 | AGCCACCUC | 1768 | 1055-1075 | GAAUCCCAUG | 195 | 1053-1075 | CCAGCCACCU | 3678 |
| | UCCAUGGGA | | | GAGAGGUGGC | | | CUCCAUGGGA | |
| | UUC | | | UGG | | | UUC | |
| 193 | UCUAAGCGC | 1769 | 2281-2301 | CUCGAGCUUG | 196 | 2279-2301 | UCUCUAAGCG | 3679 |
| | AUCAAGCUC | | | AUGCGCUUAG | | | CAUCAAGCUC | |
| | GAG | | | AGA | | | GAG | |
| 194 | AGGGAACAC | 1770 | 1353-1373 | GGUGGGCUAG | 197 | 1351-1373 | GCAGGGAACA | 3680 |
| | GUCUAGCCC | | | ACGUGUUCCC | | | CGUCUAGCCC | |
| | ACC | | | UGC | | | ACC | |
| 195 | UGUGGGCAG | 1771 | 526-546 | GUUCAUCUGG | 198 | 524-546 | CCUGUGGGCA | 3681 |
| | UGCCAGAUG | | | CACUGCCCAC | | | GUGCCAGAUG | |
| | AAC | | | AGG | | | AAC | |
| 196 | GCCACCUCU | 1772 | 1056-1076 | UGAAUCCCAU | 199 | 1054-1076 | CAGCCACCUC | 3682 |
| | CCAUGGGAU | | | GGAGAGGUGG | | | UCCAUGGGAU | |
| | UCA | | | CUG | | | UCA | |
| 197 | CCAACACGG | 1773 | 2333-2353 | AGUACACGUU | 200 | 2331-2353 | GCCCAACACG | 3684 |
| | AGAACGUGU | | | CUCCGUGUUG | | | GAGAACGUGU | |
| | ACU | | | GGC | | | ACU | |
| 198 | AAUACCAGG | 1774 | 1110-1130 | CUCGAUACUG | 201 | 1108-1130 | AGAAUACCAG | 3684 |
| | AUCAGUAUC | | | AUCCUGGUAU | | | GAUCAGUAUC | |
| | GAG | | | UCU | | | GAG | |
| 199 | CCUCCUCAC | 1775 | 516-536 | CACUGCCCAC | 202 | 514-536 | GACCUCCUCA | 3685 |
| | CUGUGGGCA | | | AGGUGAGGAG | | | CCUGUGGGCA | |
| | GUG | | | GUC | | | GUG | |
| 200 | CUUCUCUAA | 1776 | 2277-2297 | AGCUUGAUGC | 203 | 2275-2297 | CCCUUCUCUA | 3686 |
| | GCGCAUCAA | | | GCUUAGAGAA | | | AGCGCAUCAA | |
| | GCU | | | GGG | | | GCU | |
| 201 | GCAGUGCCA | 1777 | 531-551 | GGGAAGUUCA | 204 | 529-551 | GGGCAGUGCC | 3687 |
| | GAUGAACUU | | | UCUGGCACUG | | | AGAUGAACUU | |
| | CCC | | | CCC | | | CCC | |
| 202 | GAAGACGCA | 1778 | 1656-1676 | GAUUUGUGCA | 205 | 1654-1676 | AUGAAGACGC | 3688 |
| | CAUGCACAA | | | UGUGCGUCUU | | | ACAUGCACAA | |
| | AUC | | | CAU | | | AUC | |
| 203 | UGGCAGGGA | 1779 | 1349-1369 | GGCUAGACGU | 206 | 1347-1369 | GCUGGCAGGG | 3689 |
| | ACACGUCUA | | | GUUCCCUGCC | | | AACACGUCUA | |
| | GCC | | | AGC | | | GCC | |
| 204 | CUAUGGAGC | 1780 | 1304-1324 | CCAUGGCGGG | 207 | 1302-1324 | GGCUAUGGAG | 3690 |
| | CUCCCGCCA | | | AGGCUCCAUA | | | CCUCCCGCCA | |
| | UGG | | | GCC | | | UGG | |
| 205 | CACGCCAGA | 1781 | 699-719 | CAAUCGUCAU | 208 | 697-719 | GUCACGCCAG | 3691 |
| | GGAUGACGA | | | CCUCUGGCGU | | | AGGAUGACGA | |
| | UUG | | | GAC | | | UUG | |
| 206 | CCAGCCCUA | 1782 | 1391-1411 | GUAACCUUUG | 209 | 1389-1411 | GCCCAGCCCU | 3692 |
| | UGCAAAGGU | | | CAUAGGGCUG | | | AUGCAAAGGU | |
| | UAC | | | GGC | | | UAC | |
| 207 | CCAGACCAC | 1783 | 475-495 | UCCCAACGGG | 210 | 473-495 | AACCAGACCA | 3693 |
| | GGCCCGUUG | | | CCGUGGUCUG | | | CGGCCCGUUG | |
| | GGA | | | GUU | | | GGA | |
| 208 | CUCUAAGCG | 1784 | 2280-2300 | UCGAGCUUGA | 211 | 2278-2300 | UUCUCUAAGC | 3694 |
| | CAUCAAGCU | | | UGCGCUUAGA | | | GCAUCAAGCU | |
| | CGA | | | GAA | | | CGA | |
| 209 | GGACACUUG | 1785 | 2130-2150 | GAGUCUUCGU | 212 | 2128-2150 | AGGGACACUU | 3695 |
| | CGACGAAGA | | | CGCAAGUGUC | | | GCGACGAAGA | |
| | CUC | | | CCU | | | CUC | |
| 210 | GCCACAGGG | 1786 | 2123-2143 | CGUCGCAAGU | 213 | 2121-2143 | GGGCCACAGG | 3696 |
| | ACACUUGCG | | | GUCCCUGUGG | | | GACACUUGCG | |
| | ACG | | | CCC | | | ACG | |
| 211 | UCCCGUGGA | 1787 | 675-695 | UGGAUGCCAA | 214 | 673-695 | AAUCCCGUGG | 3687 |
| | GGUUGGCAU | | | CCUCCACGGG | | | AGGUUGGCAU | |
| | CCA | | | AUU | | | CCA | |
| 212 | CUGGGCGAG | 1788 | 2080-2100 | GCGCUUAUGC | 215 | 2078-2100 | UCCUGGGCGA | 3698 |
| | AAGCAUAAG | | | UUCUCGCCCA | | | GAAGCAUAAG | |
| | CGC | | | GGA | | | CGC | |
| 213 | CAACACGGA | 1789 | 2334-2354 | GAGUACACGU | 216 | 2332-2354 | CCCAACACGG | 3699 |
| | GAACGUGUA | | | UCUCCGUGUU | | | AGAACGUGUA | |
| | CUC | | | GGG | | | CUC | |
| 214 | ACCACGGCC | 1790 | 479-499 | GAGCUCCCAA | 217 | 477-499 | AGACCACGGC | 3700 |
| | CGUUGGGAG | | | CGGGCCGUGG | | | CCGUUGGGAG | |
| | CUC | | | UCU | | | CUC | |
| 215 | UCCAGGUCA | 1791 | 692-712 | CAUCCUCUGG | 218 | 690-712 | CAUCCAGGUC | 3701 |
| | CGCCAGAGG | | | CGUGACCUGG | | | ACGCCAGAGG | |
| | AUG | | | AUG | | | AUG | |
| 216 | UAGAUGACC | 1792 | 5933-5953 | GUAAUGACCU | 219 | 5931-5953 | UUUAGAUGAC | 3702 |
| | AAAGGUCAU | | | UUGGUCAUCU | | | CAAAGGUCAU | |
| | UAC | | | AAA | | | UAC | |
| 217 | GAAGCAUAA | 1793 | 2088-2108 | AGGUGGCCGC | 220 | 2086-2108 | GAGAAGCAUA | 3703 |
| | GCGCGGCCA | | | GCUUAUGCUU | | | AGCGCGGCCA | |
| | CCU | | | CUC | | | CCU | |
| 218 | AAGUGCAAC | 1794 | 1600-1620 | GUGGUCGCAC | 221 | 1598-1620 | ACAAGUGCAA | 3704 |
| | CUGUGCGAC | | | AGGUUGCACU | | | CCUGUGCGAC | |
| | CAC | | | UGU | | | CAC | |
| 219 | AGUGCAACC | 1795 | 1601-1621 | CGUGGUCGCA | 222 | 1599-1621 | CAAGUGCAAC | 3705 |
| | UGUGCGACC | | | CAGGUUGCAC | | | CUGUGCGACC | |
| | ACG | | | UUG | | | ACG | |
| 220 | CCUGCCCGA | 1796 | 2010-2030 | CCCUGCAUGA | 223 | 2008-2030 | GCCCUGCCCG | 3706 |
| | CGUCAUGCA | | | CGUCGGGCAG | | | ACGUCAUGCA | |
| | GGG | | | GGC | | | GGG | |
| 221 | CUCCUGACG | 1797 | 18-38 | UGCGAACUUG | 224 | 16-38 | GACUCCUGAC | 3707 |
| | UUCAAGUUC | | | AACGUCAGGA | | | GUUCAAGUUC | |
| | GCA | | | GUC | | | GCA | |
| 222 | GUGCAACCU | 1798 | 1602-1622 | GCGUGGUCGC | 225 | 1600-1622 | AAGUGCAACC | 3708 |
| | GUGCGACCA | | | ACAGGUUGCA | | | UGUGCGACCA | |
| | CGC | | | CUU | | | CGC | |
| 223 | GGUCCUGGG | 1799 | 2076-2096 | UUAUGCUUCU | 226 | 2074-2096 | CAGGUCCUGG | 3709 |
| | CGAGAAGCA | | | CGCCCAGGAC | | | GCGAGAAGCA | |
| | UAA | | | CUG | | | UAA | |
| 224 | GGCCACAGG | 1800 | 2122-2142 | GUCGCAAGUG | 227 | 2120-2142 | AGGGCCACAG | 3710 |
| | GACACUUGC | | | UCCCUGUGGC | | | GGACACUUGC | |
| | GAC | | | CCU | | | GAC | |
| 225 | AGAAUACCA | 1801 | 1108-1128 | CGAUACUGAU | 228 | 1106-1128 | UAAGAAUACC | 3711 |
| | GGAUCAGUA | | | CCUGGUAUUC | | | AGGAUCAGUA | |
| | UCG | | | UUA | | | UCG | |
| 226 | GAACCAGAC | 1802 | 472-492 | CAACGGGCCG | 229 | 470-492 | AUGAACCAGA | 3712 |
| | CACGGCCCG | | | UGGUCUGGUU | | | CCACGGCCCG | |
| | UUG | | | CAU | | | UUG | |
| 227 | GGAGCCUCC | 1803 | 1308-1328 | AAAUCCAUGG | 230 | 1306-1328 | AUGGAGCCUC | 3713 |
| | CGCCAUGGA | | | CGGGAGGCUC | | | CCGCCAUGGA | |
| | UUU | | | CAU | | | UUU | |
| 228 | UGAACCAGA | 1804 | 471-491 | AACGGGCCGU | 231 | 469-491 | GAUGAACCAG | 3714 |
| | CCACGGCCC | | | GGUCUGGUUC | | | ACCACGGCCC | |
| | GUU | | | AUC | | | GUU | |
| 229 | AGCCAGGUA | 1805 | 1424-1444 | AGGGCGGCUU | 232 | 1422-1444 | CCAGCCAGGU | 3715 |
| | GCAAGCCGC | | | GCUACCUGGC | | | AGCAAGCCGC | |
| | CCU | | | UGG | | | CCU | |
| 230 | AGACUCCUG | 1806 | 15-35 | GAACUUGAAC | 233 | 13-35 | CCAGACUCCU | 3716 |
| | ACGUUCAAG | | | GUCAGGAGUC | | | GACGUUCAAG | |
| | UUC | | | UGG | | | UUC | |
| 231 | UCACGCCAG | 1807 | 698-718 | AAUCGUCAUC | 234 | 696-718 | GGUCACGCCA | 3717 |
| | AGGAUGACG | | | CUCUGGCGUG | | | GAGGAUGACG | |
| | AUU | | | ACC | | | AUU | |
| 232 | GUCACGCCA | 1808 | 697-717 | AUCGUCAUCC | 235 | 695-717 | AGGUCACGCC | 3718 |
| | GAGGAUGAC | | | UCUGGCGUGA | | | AGAGGAUGAC | |
| | GAU | | | CCU | | | GAU | |
| 233 | UCCAAUCCC | 1809 | 670-690 | GCCAACCUCC | 236 | 668-690 | CAUCCAAUCC | 3719 |
| | GUGGAGGUU | | | ACGGGAUUGG | | | CGUGGAGGUU | |
| | GGC | | | AUG | | | GGC | |
| 234 | CAGACCACG | 1810 | 476-496 | CUCCCAACGG | 237 | 474-496 | ACCAGACCAC | 3720 |
| | GCCCGUUGG | | | GCCGUGGUCU | | | GGCCCGUUGG | |
| | GAG | | | GGU | | | GAG | |
| 235 | GCCAAGUUC | 1811 | 1786-1806 | GUUCUCGCUC | 238 | 1784-1806 | UGGCCAAGUU | 3721 |
| | AAGAGCGAG | | | UUGAACUUGG | | | CAAGAGCGAG | |
| | AAC | | | CCA | | | AAC | |
| 236 | AGAUGACCA | 1812 | 5934-5954 | UGUAAUGACC | 239 | 5932-5954 | UUAGAUGACC | 3722 |
| | AAGGUCAUU | | | UUUGGUCAUC | | | AAAGGUCAUU | |
| | ACA | | | UAA | | | ACA | |
| 237 | UCCCUUCCU | 1813 | 2397-2417 | UCUCCGAAGC | 240 | 2395-2417 | GAUCCCUUCC | 3723 |
| | UAGCUUCGG | | | UAAGGAAGGG | | | UUAGCUUCGG | |
| | AGA | | | AUC | | | AGA | |
| 238 | ACACGUCUA | 1814 | 1358-1378 | ACAGCGGUGG | 241 | 1356-1378 | GAACACGUCU | 3724 |
| | GCCCACCGC | | | GCUAGACGUG | | | AGCCCACCGC | |
| | UGU | | | UUC | | | UGU | |
| 239 | GUGGUGGCC | 1815 | 1780-1800 | GCUCUUGAAC | 242 | 1778-1800 | CCGUGGUGGC | 3725 |
| | AAGUUCAAG | | | UUGGCCACCA | | | CAAGUUCAAG | |
| | AGC | | | CGG | | | AGC | |
| 240 | CCCUUCCUU | 1816 | 2398-2418 | GUCUCCGAAG | 243 | 2396-2418 | AUCCCUUCCU | 3726 |
| | AGCUUCGGA | | | CUAAGGAAGG | | | UAGCUUCGGA | |
| | GAC | | | GAU | | | GAC | |
| 241 | UGCCCAACA | 1817 | 2330-2350 | ACACGUUCUC | 244 | 2328-2350 | GAUGCCCAAC | 3727 |
| | CGGAGAACG | | | CGUGUUGGGC | | | ACGGAGAACG | |
| | UGU | | | AUC | | | UGU | |
| 242 | GUAUCGAGA | 1818 | 1123-1143 | GCCGGAAGCC | 245 | 1121-1143 | CAGUAUCGAG | 3728 |
| | GAGGCUUCC | | | UCUCUCGAUA | | | AGAGGCUUCC | |
| | GGC | | | CUG | | | GGC | |
| 243 | CGUCUAGCC | 1819 | 1361-1381 | GGGACAGCGG | 246 | 1359-1381 | CACGUCUAGC | 3729 |
| | CACCGCUGU | | | UGGGCUAGAC | | | CCACCGCUGU | |
| | CCC | | | GUG | | | CCC | |
| 244 | AGGUUGGCA | 1820 | 683-703 | GCGUGACCUG | 247 | 681-703 | GGAGGUUGGC | 3730 |
| | UCCAGGUCA | | | GAUGCCAACC | | | AUCCAGGUCA | |
| | CGC | | | UCC | | | CGC | |
| 245 | UAUGGAGCC | 1821 | 1305-1325 | UCCAUGGCGG | 248 | 1303-1325 | GCUAUGGAGC | 3731 |
| | UCCCGCCAU | | | GAGGCUCCAU | | | CUCCCGCCAU | |
| | GGA | | | AGC | | | GGA | |
| 246 | AAUCCCGUG | 1822 | 673-693 | GAUGCCAACC | 249 | 671-693 | CCAAUCCCGU | 3732 |
| | GAGGUUGGC | | | UCCACGGGAU | | | GGAGGUUGGC | |
| | AUC | | | UGG | | | AUC | |
| 247 | UUGGCAUCC | 1823 | 686-706 | CUGGCGUGAC | 250 | 684-706 | GGUUGGCAUC | 3733 |
| | AGGUCACGC | | | CUGGAUGCCA | | | CAGGUCACGC | |
| | CAG | | | ACC | | | CAG | |
| 248 | CUUAGCUUC | 1824 | 2404-2424 | UCUGGAGUCU | 251 | 2402-2424 | UCCUUAGCUU | 3734 |
| | GGAGACUCC | | | CCGAAGCUAA | | | CGGAGACUCC | |
| | AGA | | | GGA | | | AGA | |
| 249 | CACGUCUAG | 1825 | 1359-1379 | GACAGCGGUG | 252 | 1357-1379 | AACACGUCUA | 3735 |
| | CCCACCGCU | | | GGCUAGACGU | | | GCCCACCGCU | |
| | GUC | | | GUU | | | GUC | |
| 250 | AACCAGACC | 1826 | 473-493 | CCAACGGGCC | 253 | 471-493 | UGAACCAGAC | 3736 |
| | ACGGCCCGU | | | GUGGUCUGGU | | | CACGGCCCGU | |
| | UGG | | | UCA | | | UGG | |
| 251 | AACACGUCU | 1827 | 1357-1377 | CAGCGGUGGG | 254 | 1355-1377 | GGAACACGUC | 3737 |
| | AGCCCACCG | | | CUAGACGUGU | | | UAGCCCACCG | |
| | CUG | | | UCC | | | CUG | |
| 252 | GUCCAUCCA | 1828 | 7-27 | ACGUCAGGAG | 255 | 5-27 | CUGUCCAUCC | 3738 |
| | GACUCCUGA | | | UCUGGAUGGA | | | AGACUCCUGA | |
| | CGU | | | CAG | | | CGU | |
| 253 | AGCUGGACG | 1829 | 2498-2518 | CCGAGAUCCC | 256 | 2496-2518 | GGAGCUGGAC | 3739 |
| | GAGGGAUCU | | | UCCGUCCAGC | | | GGAGGGAUCU | |
| | CGG | | | UCC | | | CGG | |
| 254 | CUAAGCGCA | 1830 | 2282-2302 | UCUCGAGCUU | 257 | 2280-2302 | CUCUAAGCGC | 3740 |
| | UCAAGCUCG | | | GAUGCGCUUA | | | AUCAAGCUCG | |
| | AGA | | | GAG | | | AGA | |
| 255 | GUUGGCAUC | 1831 | 685-705 | UGGCGUGACC | 258 | 683-705 | AGGUUGGCAU | 3741 |
| | CAGGUCACG | | | UGGAUGCCAA | | | CCAGGUCACG | |
| | CCA | | | CCU | | | CCA | |
| 256 | CCAGGUAGC | 1832 | 1426-1446 | GAAGGGCGGC | 259 | 1424-1446 | AGCCAGGUAG | 3742 |
| | AAGCCGCCC | | | UUGCUACCUG | | | CAAGCCGCCC | |
| | UUC | | | GCU | | | UUC | |
| 257 | CCAGCCAGG | 1833 | 1422-1442 | GGCGGCUUGC | 260 | 1420-1442 | UUCCAGCCAG | 3743 |
| | UAGCAAGCC | | | UACCUGGCUG | | | GUAGCAAGCC | |
| | GCC | | | GAA | | | GCC | |
| 258 | AGGGACACU | 1834 | 2128-2148 | GUCUUCGUCG | 261 | 2126-2148 | ACAGGGACAC | 3744 |
| | UGCGACGAA | | | CAAGUGUCCC | | | UUGCGACGAA | |
| | GAC | | | UGU | | | GAC | |
| 259 | ACAAGUGCA | 1835 | 1598-1618 | GGUCGCACAG | 262 | 1596-1618 | CUACAAGUGC | 3745 |
| | ACCUGUGCG | | | GUUGCACUUG | | | AACCUGUGCG | |
| | ACC | | | UAG | | | ACC | |
| 260 | GCUGGACGG | 1836 | 2499-2519 | CCCGAGAUCC | 263 | 2497-2519 | GAGCUGGACG | 3746 |
| | AGGGAUCUC | | | CUCCGUCCAG | | | GAGGGAUCUC | |
| | GGG | | | CUC | | | GGG | |
| 261 | CAAAUUUCA | 1837 | 1542-1562 | ACCAGGUUGC | 264 | 1540-1562 | UUCAAAUUUC | 3747 |
| | GAGCAACCU | | | UCUGAAAUUU | | | AGAGCAACCU | |
| | GGU | | | GAA | | | GGU | |
| 262 | CUGUCCAUC | 1838 | 5-25 | GUCAGGAGUC | 265 | 3-25 | CUCUGUCCAU | 3748 |
| | CAGACUCCU | | | UGGAUGGACA | | | CCAGACUCCU | |
| | GAC | | | GAG | | | GAC | |
| 263 | AUCCCGUGG | 1839 | 674-694 | GGAUGCCAAC | 266 | 672-694 | CAAUCCCGUG | 3749 |
| | AGGUUGGCA | | | CUCCACGGGA | | | GAGGUUGGCA | |
| | UCC | | | UUG | | | UCC | |
| 264 | UCCUGGGCG | 1840 | 2078-2098 | GCUUAUGCUU | 267 | 2076-2098 | GGUCCUGGGC | 3750 |
| | AGAAGCAUA | | | CUCGCCCAGG | | | GAGAAGCAUA | |
| | AGC | | | ACC | | | AGC | |
| 265 | UAUCGAGAG | 1841 | 1124-1144 | GGCCGGAAGC | 268 | 1122-1144 | AGUAUCGAGA | 3751 |
| | AGGCUUCCG | | | CUCUCUCGAU | | | GAGGCUUCCG | |
| | GCC | | | ACU | | | GCC | |
| 266 | CCGACGUCA | 1842 | 2015-2035 | CCAUGCCCUG | 269 | 2013-2035 | GCCCGACGUC | 3752 |
| | UGCAGGGCA | | | CAUGACGUCG | | | AUGCAGGGCA | |
| | UGG | | | GGC | | | UGG | |
| 267 | UGUCCAUCC | 1843 | 6-26 | CGUCAGGAGU | 270 | 4-26 | UCUGUCCAUC | 3753 |
| | AGACUCCUG | | | CUGGAUGGAC | | | CAGACUCCUG | |
| | ACG | | | AGA | | | ACG | |
| 268 | AGGGCCACA | 1844 | 2120-2140 | CGCAAGUGUC | 271 | 2118-2140 | CGAGGGCCAC | 3754 |
| | GGGACACUU | | | CCUGUGGCCC | | | AGGGACACUU | |
| | GCG | | | UCG | | | GCG | |
| 269 | ACCAGACCA | 1845 | 474-494 | CCCAACGGGC | 272 | 472-494 | GAACCAGACC | 3755 |
| | CGGCCCGUU | | | CGUGGUCUGG | | | ACGGCCCGUU | |
| | GGG | | | UUC | | | GGG | |
| 270 | GCAUAAGCG | 1846 | 2091-2111 | GCCAGGUGGC | 273 | 2089-2111 | AAGCAUAAGC | 3756 |
| | CGGCCACCU | | | CGCGCUUAUG | | | GCGGCCACCU | |
| | GGC | | | CUU | | | GGC | |
| 271 | AUGGCUAUG | 1847 | 1300-1320 | GGCGGGAGGC | 274 | 1298-1320 | CAAUGGCUAU | 3757 |
| | GAGCCUCCC | | | UCCAUAGCCA | | | GGAGCCUCCC | |
| | GCC | | | UUG | | | GCC | |
| 272 | CAGGAACAC | 1848 | 754-774 | UUUAUCUGCU | 275 | 752-774 | AACAGGAACA | 3758 |
| | AUAGCAGAU | | | AUGUGUUCCU | | | CAUAGCAGAU | |
| | AAA | | | GUU | | | AAA | |
| 273 | AGGAACACA | 1849 | 755-775 | GUUUAUCUGC | 276 | 753-775 | ACAGGAACAC | 3759 |
| | UAGCAGAUA | | | UAUGUGUUCC | | | AUAGCAGAUA | |
| | AAC | | | UGU | | | AAC | |
| 274 | GGAACACAU | 1850 | 756-776 | AGUUUAUCUG | 277 | 754-776 | CAGGAACACA | 3760 |
| | AGCAGAUAA | | | CUAUGUGUUC | | | UAGCAGAUAA | |
| | ACU | | | CUG | | | ACU | |
| 275 | GAACACAUA | 1851 | 757-777 | AAGUUUAUCU | 278 | 755-777 | AGGAACACAU | 3761 |
| | GCAGAUAAA | | | GCUAUGUGUU | | | AGCAGAUAAA | |
| | CUU | | | CCU | | | CUU | |
| 276 | AACACAUAG | 1852 | 758-778 | GAAGUUUAUC | 279 | 756-778 | GGAACACAUA | 3762 |
| | CAGAUAAAC | | | UGCUAUGUGU | | | GCAGAUAAAC | |
| | UUC | | | UCC | | | UUC | |
| 277 | CCCAGCAGC | 1853 | 883-903 | UGUACAUGUG | 280 | 881-903 | AGCCCAGCAG | 3763 |
| | UACACAUGU | | | UAGCUGCUGG | | | CUACACAUGU | |
| | ACA | | | GCU | | | ACA | |
| 278 | CAGCAGCUA | 1854 | 885-905 | GUUGUACAUG | 281 | 883-905 | CCCAGCAGCU | 3764 |
| | CACAUGUAC | | | UGUAGCUGCU | | | ACACAUGUAC | |
| | AAC | | | GGG | | | AAC | |
| 279 | GCAGCUACA | 1855 | 887-907 | AAGUUGUACA | 282 | 885-907 | CAGCAGCUAC | 3765 |
| | CAUGUACAA | | | UGUGUAGCUG | | | ACAUGUACAA | |
| | CUU | | | CUG | | | CUU | |
| 280 | AGCUACACA | 1856 | 889-909 | GCAAGUUGUA | 283 | 887-909 | GCAGCUACAC | 3766 |
| | UGUACAACU | | | CAUGUGUAGC | | | AUGUACAACU | |
| | UGC | | | UGC | | | UGC | |
| 281 | CUACACAUG | 1857 | 891-911 | UUGCAAGUUG | 284 | 889-911 | AGCUACACAU | 3767 |
| | UACAACUUG | | | UACAUGUGUA | | | GUACAACUUG | |
| | CAA | | | GCU | | | CAA | |
| 282 | UACACAUGU | 1858 | 892-912 | UUUGCAAGUU | 285 | 890-912 | GCUACACAUG | 3768 |
| | ACAACUUGC | | | GUACAUGUGU | | | UACAACUUGC | |
| | AAA | | | AGC | | | AAA | |
| 283 | ACAUGUACA | 1859 | 895-915 | AUGUUUGCAA | 286 | 893-915 | ACACAUGUAC | 3769 |
| | ACUUGCAAA | | | GUUGUACAUG | | | AACUUGCAAA | |
| | CAU | | | UGU | | | CAG | |
| 284 | CAUGUACAA | 1860 | 896-916 | GCUGUUUGCA | 287 | 894-916 | CACAUGUACA | 3770 |
| | CUUGCAAAC | | | AGUUGUACAU | | | ACUUGCAAAC | |
| | AGC | | | GUG | | | AGC | |
| 285 | AUGUACAAC | 1861 | 897-917 | GGCUGUUUGC | 288 | 895-917 | ACAUGUACAA | 3552 |
| | UUGCAAACA | | | AAGUUGUACA | | | CUUGCAAACA | |
| | GCC | | | UGU | | | GCC | |
| 286 | UGUACAACU | 1862 | 898-918 | UGGCUGUUUG | 289 | 896-918 | CAUGUACAAC | 3771 |
| | UGCAAACAG | | | CAAGUUGUAC | | | UUGCAAACAG | |
| | CCA | | | AUG | | | CCA | |
| 287 | GUACAACUU | 1863 | 899-919 | AUGGCUGUUU | 290 | 897-919 | AUGUACAACU | 3772 |
| | GCAAACAGC | | | GCAAGUUGUA | | | UGCAAACAGC | |
| | CAU | | | CAU | | | CAU | |
| 288 | UACAACUUG | 1864 | 900-920 | AAUGGCUGUU | 291 | 898-920 | UGUACAACUU | 3773 |
| | CAAACAGCC | | | UGCAAGUUGU | | | GCAAACAGCC | |
| | AUU | | | ACA | | | AUU | |
| 289 | ACAACUUGC | 1865 | 901-921 | GAAUGGCUGU | 292 | 899-921 | GUACAACUUG | 3774 |
| | AAACAGCCA | | | UUGCAAGUUG | | | CAAACAGCCA | |
| | UUC | | | UAC | | | UUC | |
| 290 | CAACUUGCA | 1866 | 902-922 | UGAAUGGCUG | 293 | 900-922 | UACAACUUGC | 3775 |
| | AACAGCCAU | | | UUUGCAAGUU | | | AAACAGCCAU | |
| | UCA | | | GUA | | | UCA | |
| 291 | AACUUGCAA | 1867 | 903-923 | GUGAAUGGCU | 294 | 901-923 | ACAACUUGCA | 3776 |
| | ACAGCCAUU | | | GUUUGCAAGU | | | AACAGCCAUU | |
| | CAC | | | UGU | | | CAC | |
| 292 | ACUUGCAAA | 1868 | 904-924 | GGUGAAUGGC | 295 | 902-924 | CAACUUGCAA | 3556 |
| | CAGCCAUUC | | | UGUUUGCAAG | | | ACAGCCAUUC | |
| | ACC | | | UUG | | | ACC | |
| 293 | CUUGCAAAC | 1869 | 905-925 | UGGUGAAUGG | 296 | 903-925 | AACUUGCAAA | 3778 |
| | AGCCAUUCA | | | CUGUUUGCAA | | | CAGCCAUUCA | |
| | CCA | | | GUU | | | CCA | |
| 294 | UUGCAAACA | 1870 | 906-926 | AUGGUGAAUG | 297 | 904-926 | ACUUGCAAAC | 3779 |
| | GCCAUUCAC | | | GCUGUUUGCA | | | AGCCAUUCAC | |
| | CAU | | | AGU | | | CAG | |
| 295 | UGCAAACAG | 1871 | 907-927 | ACUGGUGAAU | 298 | 905-927 | CUUGCAAACA | 3780 |
| | CCAUUCACC | | | GGCUGUUUGC | | | GCCAUUCACC | |
| | AGU | | | AAG | | | AGU | |
| 296 | GCAAACAGC | 1872 | 908-928 | CACUGGUGAA | 299 | 906-928 | UUGCAAACAG | 3551 |
| | CAUUCACCA | | | UGGCUGUUUG | | | CCAUUCACCA | |
| | GUG | | | CAA | | | GUG | |
| 297 | CAAACAGCC | 1873 | 909-929 | GCACUGGUGA | 300 | 907-929 | UGCAAACAGC | 3781 |
| | AUUCACCAG | | | AUGGCUGUUU | | | CAUUCACCAG | |
| | UGC | | | GCA | | | UGC | |
| 298 | AAACAGCCA | 1874 | 910-930 | UGCACUGGUG | 301 | 908-930 | GCAAACAGCC | 3782 |
| | UUCACCAGU | | | AAUGGCUGUU | | | AUUCACCAGU | |
| | GCA | | | UGC | | | GCA | |
| 299 | AACAGCCAU | 1875 | 911-931 | AUGCACUGGU | 302 | 909-931 | CAAACAGCCA | 3783 |
| | UCACCAGUG | | | GAAUGGCUGU | | | UUCACCAGUG | |
| | CAU | | | UUG | | | CAU | |
| 300 | ACAGCCAUU | 1876 | 912-932 | AAUGCACUGG | 303 | 910-932 | AAACAGCCAU | 3784 |
| | CACCAGUGC | | | UGAAUGGCUG | | | UCACCAGUGC | |
| | AUU | | | UUU | | | AUG | |
| 301 | CAGCCAUUC | 1877 | 913-933 | ACAUGCACUG | 304 | 911-933 | AACAGCCAUU | 3785 |
| | ACCAGUGCA | | | GUGAAUGGCU | | | CACCAGUGCA | |
| | UGU | | | GUU | | | UGG | |
| 302 | AGCCAUUCA | 1878 | 914-934 | ACCAUGCACU | 305 | 912-934 | ACAGCCAUUC | 3786 |
| | CCAGUGCAU | | | GGUGAAUGGC | | | ACCAGUGCAU | |
| | GGU | | | UGU | | | GGU | |
| 303 | GCCAUUCAC | 1879 | 915-935 | AACCAUGCAC | 306 | 913-935 | CAGCCAUUCA | 3787 |
| | CAGUGCAUG | | | UGGUGAAUGG | | | CCAGUGCAUG | |
| | GUU | | | CUG | | | GUU | |
| 304 | CCAUUCACC | 1880 | 916-936 | AAACCAUGCA | 307 | 914-936 | AGCCAUUCAC | 3788 |
| | AGUGCAUGG | | | CUGGUGAAUG | | | CAGUGCAUGG | |
| | UUU | | | GCU | | | UUU | |
| 305 | CAUUCACCA | 1881 | 917-937 | GAAACCAUGC | 308 | 915-937 | GCCAUUCACC | 3789 |
| | GUGCAUGGU | | | ACUGGUGAAU | | | AGUGCAUGGU | |
| | UUC | | | GGC | | | UUC | |
| 306 | AUUCACCAG | 1882 | 918-938 | AGAAACCAUG | 309 | 916-938 | CCAUUCACCA | 3790 |
| | UGCAUGGUU | | | CACUGGUGAA | | | GUGCAUGGUU | |
| | UCU | | | UGG | | | UCU | |
| 307 | UUCACCAGU | 1883 | 919-939 | GAGAAACCAU | 310 | 917-939 | CAUUCACCAG | 3791 |
| | GCAUGGUUU | | | GCACUGGUGA | | | UGCAUGGUUU | |
| | CUC | | | AUG | | | CUC | |
| 308 | UCACCAGUG | 1884 | 920-940 | AGAGAAACCA | 311 | 918-940 | AUUCACCAGU | 3792 |
| | CAUGGUUUC | | | UGCACUGGUG | | | GCAUGGUUUC | |
| | UCU | | | AAU | | | UCU | |
| 309 | CACCAGUGC | 1885 | 921-941 | AAGAGAAACC | 312 | 919-941 | UUCACCAGUG | 3793 |
| | AUGGUUUCU | | | AUGCACUGGU | | | CAUGGUUUCU | |
| | CUU | | | GAA | | | CUU | |
| 310 | ACCAGUGCA | 1886 | 922-942 | AAAGAGAAAC | 313 | 920-942 | UCACCAGUGC | 3794 |
| | UGGUUUCUC | | | CAUGCACUGG | | | AUGGUUUCUC | |
| | UUU | | | UGA | | | UUG | |
| 311 | GGUUUCUCU | 1887 | 932-952 | GUGCGUGUUG | 314 | 930-952 | AUGGUUUCUC | 3795 |
| | UGCAACACG | | | CAAGAGAAAC | | | UUGCAACACG | |
| | CAC | | | CAU | | | CAC | |
| 312 | GUUUCUCUU | 1888 | 933-953 | UGUGCGUGUU | 315 | 931-953 | UGGUUUCUCU | 3796 |
| | GCAACACGC | | | GCAAGAGAAA | | | UGCAACACGC | |
| | ACA | | | CCA | | | ACA | |
| 313 | UUCUCUUGC | 1889 | 935-955 | UCUGUGCGUG | 316 | 933-955 | GUUUCUCUUG | 3797 |
| | AACACGCAC | | | UUGCAAGAGA | | | CAACACGCAC | |
| | AGA | | | AAC | | | AGA | |
| 314 | UCUCUUGCA | 1890 | 936-956 | UUCUGUGCGU | 317 | 934-956 | UUUCUCUUGC | 3798 |
| | ACACGCACA | | | GUUGCAAGAG | | | AACACGCACA | |
| | GAA | | | AAA | | | GAA | |
| 315 | CUUGCAACA | 1891 | 939-959 | GUGUUCUGUG | 318 | 937-959 | CUCUUGCAAC | 3799 |
| | CGCACAGAA | | | CGUGUUGCAA | | | ACGCACAGAA | |
| | CAC | | | GAG | | | CAC | |
| 316 | UUGCAACAC | 1892 | 940-960 | AGUGUUCUGU | 319 | 938-960 | UCUUGCAACA | 3800 |
| | GCACAGAAC | | | GCGUGUUGCA | | | CGCACAGAAC | |
| | ACU | | | AGA | | | ACU | |
| 317 | GCAACACGC | 1893 | 942-962 | UGAGUGUUCU | 320 | 940-962 | UUGCAACACG | 3801 |
| | ACAGAACAC | | | GUGCGUGUUG | | | CACAGAACAC | |
| | UCA | | | CAA | | | UCA | |
| 318 | CAACACGCA | 1894 | 943-963 | AUGAGUGUUC | 321 | 941-963 | UGCAACACGC | 3802 |
| | CAGAACACU | | | UGUGCGUGUU | | | ACAGAACACU | |
| | CAU | | | GCA | | | CAU | |
| 319 | ACACGCACA | 1895 | 945-965 | ACAUGAGUGU | 322 | 943-965 | CAACACGCAC | 3803 |
| | GAACACUCA | | | UCUGUGCGUG | | | AGAACACUCA | |
| | UGU | | | UUG | | | UGG | |
| 320 | CACGCACAG | 1896 | 946-966 | UCCAUGAGUG | 323 | 944-966 | AACACGCACA | 3804 |
| | AACACUCAU | | | UUCUGUGCGU | | | GAACACUCAU | |
| | GGA | | | GUU | | | GGA | |
| 321 | ACGCACAGA | 1897 | 947-967 | AUCCAUGAGU | 324 | 945-967 | ACACGCACAG | 3805 |
| | ACACUCAUG | | | GUUCUGUGCG | | | AACACUCAUG | |
| | GAU | | | UGU | | | GAU | |
| 322 | CGCACAGAA | 1898 | 948-968 | AAUCCAUGAG | 325 | 946-968 | CACGCACAGA | 3557 |
| | CACUCAUGG | | | UGUUCUGUGC | | | ACACUCAUGG | |
| | AUU | | | GUG | | | AUU | |
| 323 | GCACAGAAC | 1899 | 949-969 | UAAUCCAUGA | 326 | 947-969 | ACGCACAGAA | 3806 |
| | ACUCAUGGA | | | GUGUUCUGUG | | | CACUCAUGGA | |
| | UUA | | | CGU | | | UUA | |
| 324 | CACAGAACA | 1900 | 950-970 | UUAAUCCAUG | 327 | 948-970 | CGCACAGAAC | 3807 |
| | CUCAUGGAU | | | AGUGUUCUGU | | | ACUCAUGGAU | |
| | UAA | | | GCG | | | UAA | |
| 325 | ACAGAACAC | 1901 | 951-971 | AUUAAUCCAU | 328 | 949-971 | GCACAGAACA | 3808 |
| | UCAUGGAUU | | | GAGUGUUCUG | | | CUCAUGGAUU | |
| | AAU | | | UGC | | | AAG | |
| 326 | CAGAACACU | 1902 | 952-972 | UCUUAAUCCA | 329 | 950-972 | CACAGAACAC | 3809 |
| | CAUGGAUUA | | | UGAGUGUUCU | | | UCAUGGAUUA | |
| | AGA | | | GUG | | | AGA | |
| 327 | AGAACACUC | 1903 | 953-973 | UUCUUAAUCC | 330 | 951-973 | ACAGAACACU | 3810 |
| | AUGGAUUAA | | | AUGAGUGUUC | | | CAUGGAUUAA | |
| | GAA | | | UGU | | | GAA | |
| 328 | GAACACUCA | 1904 | 954-974 | AUUCUUAAUC | 331 | 952-974 | CAGAACACUC | 3811 |
| | UGGAUUAAG | | | CAUGAGUGUU | | | AUGGAUUAAG | |
| | AAU | | | CUG | | | AAU | |
| 329 | AACACUCAU | 1905 | 955-975 | GAUUCUUAAU | 332 | 953-975 | AGAACACUCA | 3812 |
| | GGAUUAAGA | | | CCAUGAGUGU | | | UGGAUUAAGA | |
| | AUC | | | UCU | | | AUC | |
| 330 | UUAAGAAUC | 1906 | 967-987 | GCUUUCUAAG | 333 | 965-987 | GAUUAAGAAU | 3813 |
| | UACUUAGAA | | | UAGAUUCUUA | | | CUACUUAGAA | |
| | AGC | | | AUC | | | AGC | |
| 331 | GGUUGGUAU | 1907 | 1014-1034 | AGUCCUGAAG | 334 | 1012-1034 | CGGGUUGGUA | 3814 |
| | CCCUUCAGG | | | GGAUACCAAC | | | UCCCUUCAGG | |
| | ACU | | | CCG | | | ACU | |
| 332 | GUUGGUAUC | 1908 | 1015-1035 | UAGUCCUGAA | 335 | 1013-1035 | GGGUUGGUAU | 3815 |
| | CCUUCAGGA | | | GGGAUACCAA | | | CCCUUCAGGA | |
| | CUA | | | CCC | | | CUA | |
| 333 | UUGGUAUCC | 1909 | 1016-1036 | AUAGUCCUGA | 336 | 1014-1036 | GGUUGGUAUC | 3816 |
| | CUUCAGGAC | | | AGGGAUACCA | | | CCUUCAGGAC | |
| | UAU | | | ACC | | | UAG | |
| 334 | UGGUAUCCC | 1910 | 1017-1037 | ACUAGUCCUG | 337 | 1015-1037 | GUUGGUAUCC | 3817 |
| | UUCAGGACU | | | AAGGGAUACC | | | CUUCAGGACU | |
| | AGU | | | AAC | | | AGG | |
| 335 | GGUAUCCCU | 1911 | 1018-1038 | ACCUAGUCCU | 338 | 1016-1038 | UUGGUAUCCC | 3818 |
| | UCAGGACUA | | | GAAGGGAUAC | | | UUCAGGACUA | |
| | GGU | | | CAA | | | GGU | |
| 336 | AUCCCUUCA | 1912 | 1021-1041 | UGCACCUAGU | 339 | 1019-1041 | GUAUCCCUUC | 3819 |
| | GGACUAGGU | | | CCUGAAGGGA | | | AGGACUAGGU | |
| | GCA | | | UAC | | | GCA | |
| 337 | CCUUCAGGA | 1913 | 1024-1044 | UUCUGCACCU | 340 | 1022-1044 | UCCCUUCAGG | 3820 |
| | CUAGGUGCA | | | AGUCCUGAAG | | | ACUAGGUGCA | |
| | GAA | | | GGA | | | GAA | |
| 338 | UUCAGGACU | 1914 | 1026-1046 | AAUUCUGCAC | 341 | 1024-1046 | CCUUCAGGAC | 3821 |
| | AGGUGCAGA | | | CUAGUCCUGA | | | UAGGUGCAGA | |
| | AUU | | | AGG | | | AUG | |
| 339 | UCAGGACUA | 1915 | 1027-1047 | ACAUUCUGCA | 342 | 1025-1047 | CUUCAGGACU | 3822 |
| | GGUGCAGAA | | | CCUAGUCCUG | | | AGGUGCAGAA | |
| | UGU | | | AAG | | | UGU | |
| 340 | CAGGACUAG | 1916 | 1028-1048 | GACAUUCUGC | 343 | 1026-1048 | UUCAGGACUA | 3823 |
| | GUGCAGAAU | | | ACCUAGUCCU | | | GGUGCAGAAU | |
| | GUC | | | GAA | | | GUC | |
| 341 | AGGACUAGG | 1917 | 1029-1049 | GGACAUUCUG | 344 | 1027-1049 | UCAGGACUAG | 3824 |
| | UGCAGAAUG | | | CACCUAGUCC | | | GUGCAGAAUG | |
| | UCC | | | UGA | | | UCC | |
| 342 | GGACUAGGU | 1918 | 1030-1050 | AGGACAUUCU | 345 | 1028-1050 | CAGGACUAGG | 3825 |
| | GCAGAAUGU | | | GCACCUAGUC | | | UGCAGAAUGU | |
| | CCU | | | CUG | | | CCU | |
| 343 | GACUAGGUG | 1919 | 1031-1051 | AAGGACAUUC | 346 | 1029-1051 | AGGACUAGGU | 3826 |
| | CAGAAUGUC | | | UGCACCUAGU | | | GCAGAAUGUC | |
| | CUU | | | CCU | | | CUU | |
| 344 | CACCUCUCC | 1920 | 1058-1078 | UAUGAAUCCC | 347 | 1056-1078 | GCCACCUCUC | 3827 |
| | AUGGGAUUC | | | AUGGAGAGGU | | | CAUGGGAUUC | |
| | AUA | | | GGC | | | AUA | |
| 345 | ACCUCUCCA | 1921 | 1059-1079 | AUAUGAAUCC | 348 | 1057-1079 | CCACCUCUCC | 3828 |
| | UGGGAUUCA | | | CAUGGAGAGG | | | AUGGGAUUCA | |
| | UAU | | | UGG | | | UAU | |
| 346 | CCUCUCCAU | 1922 | 1060-1080 | AAUAUGAAUC | 349 | 1058-1080 | CACCUCUCCA | 3829 |
| | GGGAUUCAU | | | CCAUGGAGAG | | | UGGGAUUCAU | |
| | AUU | | | GUG | | | AUU | |
| 347 | CUCUCCAUG | 1923 | 1061-1081 | AAAUAUGAAU | 350 | 1059-1081 | ACCUCUCCAU | 3830 |
| | GGAUUCAUA | | | CCCAUGGAGA | | | GGGAUUCAUA | |
| | UUU | | | GGU | | | UUG | |
| 348 | UCUCCAUGG | 1924 | 1062-1082 | GCAAUAUGAA | 351 | 1060-1082 | CCUCUCCAUG | 3831 |
| | GAUUCAUAU | | | UCCCAUGGAG | | | GGAUUCAUAU | |
| | UGC | | | AGG | | | UGC | |
| 349 | CUCCAUGGG | 1925 | 1063-1083 | UGCAAUAUGA | 352 | 1061-1083 | CUCUCCAUGG | 3832 |
| | AUUCAUAUU | | | AUCCCAUGGA | | | GAUUCAUAUU | |
| | GCA | | | GAG | | | GCA | |
| 350 | UCCAUGGGA | 1926 | 1064-1084 | AUGCAAUAUG | 353 | 1062-1084 | UCUCCAUGGG | 3833 |
| | UUCAUAUUG | | | AAUCCCAUGG | | | AUUCAUAUUG | |
| | CAU | | | AGA | | | CAG | |
| 351 | CCAUGGGAU | 1927 | 1065-1085 | UCUGCAAUAU | 354 | 1063-1085 | CUCCAUGGGA | 3834 |
| | UCAUAUUGC | | | GAAUCCCAUG | | | UUCAUAUUGC | |
| | AGA | | | GAG | | | AGA | |
| 352 | CAUGGGAUU | 1928 | 1066-1086 | GUCUGCAAUA | 355 | 1064-1086 | UCCAUGGGAU | 3835 |
| | CAUAUUGCA | | | UGAAUCCCAU | | | UCAUAUUGCA | |
| | GAC | | | GGA | | | GAC | |
| 353 | AUGGGAUUC | 1929 | 1067-1087 | UGUCUGCAAU | 356 | 1065-1087 | CCAUGGGAUU | 3836 |
| | AUAUUGCAG | | | AUGAAUCCCA | | | CAUAUUGCAG | |
| | ACA | | | UGG | | | ACA | |
| 354 | UGGGAUUCA | 1930 | 1068-1088 | UUGUCUGCAA | 357 | 1066-1088 | CAUGGGAUUC | 3837 |
| | UAUUGCAGA | | | UAUGAAUCCC | | | AUAUUGCAGA | |
| | CAA | | | AUG | | | CAA | |
| 355 | GGAUUCAUA | 1931 | 1070-1090 | UAUUGUCUGC | 358 | 1068-1090 | UGGGAUUCAU | 3838 |
| | UUGCAGACA | | | AAUAUGAAUC | | | AUUGCAGACA | |
| | AUA | | | CCA | | | AUA | |
| 356 | GAUUCAUAU | 1932 | 1071-1091 | UUAUUGUCUG | 359 | 1069-1091 | GGGAUUCAUA | 3839 |
| | UGCAGACAA | | | CAAUAUGAAU | | | UUGCAGACAA | |
| | UAA | | | CCC | | | UAA | |
| 357 | AUUCAUAUU | 1933 | 1072-1092 | GUUAUUGUCU | 360 | 1070-1092 | GGAUUCAUAU | 3840 |
| | GCAGACAAU | | | GCAAUAUGAA | | | UGCAGACAAU | |
| | AAC | | | UCC | | | AAC | |
| 358 | UUCAUAUUG | 1934 | 1073-1093 | GGUUAUUGUC | 361 | 1071-1093 | GAUUCAUAUU | 3841 |
| | CAGACAAUA | | | UGCAAUAUGA | | | GCAGACAAUA | |
| | ACC | | | AUC | | | ACC | |
| 359 | UCAUAUUGC | 1935 | 1074-1094 | GGGUUAUUGU | 362 | 1072-1094 | AUUCAUAUUG | 3842 |
| | AGACAAUAA | | | CUGCAAUAUG | | | CAGACAAUAA | |
| | CCC | | | AAU | | | CCC | |
| 360 | CUUUAACCU | 1936 | 1095-1115 | GGUAUUCUUA | 363 | 1093-1115 | CCCUUUAACC | 3843 |
| | GCUAAGAAU | | | GCAGGUUAAA | | | UGCUAAGAAU | |
| | ACC | | | GGG | | | ACC | |
| 361 | UUUAACCUG | 1937 | 1096-1116 | UGGUAUUCUU | 364 | 1094-1116 | CCUUUAACCU | 3844 |
| | CUAAGAAUA | | | AGCAGGUUAA | | | GCUAAGAAUA | |
| | CCA | | | AGG | | | CCA | |
| 362 | UUAACCUGC | 1938 | 1097-1117 | AUGGUAUUCU | 365 | 1095-1117 | CUUUAACCUG | 3845 |
| | UAAGAAUAC | | | UAGCAGGUUA | | | CUAAGAAUAC | |
| | CAU | | | AAG | | | CA | |
| 363 | UAACCUGCU | 1939 | 1098-1118 | ACUGGUAUUC | 366 | 1096-1118 | UUUAACCUGC | 3846 |
| | AAGAAUACC | | | UUAGCAGGUU | | | UAAGAAUACC | |
| | AGU | | | AAA | | | AGG | |
| 364 | AACCUGCUA | 1940 | 1099-1119 | UCCUGGUAUU | 367 | 1097-1119 | UUAACCUGCU | 3847 |
| | AGAAUACCA | | | CUUAGCAGGU | | | AAGAAUACCA | |
| | GGA | | | UAA | | | GGA | |
| 365 | ACCUGCUAA | 1941 | 1100-1120 | AUCCUGGUAU | 368 | 1098-1120 | UAACCUGCUA | 3848 |
| | GAAUACCAG | | | UCUUAGCAGG | | | AGAAUACCAG | |
| | GAU | | | UUA | | | GAU | |
| 366 | CCUGCUAAG | 1942 | 1101-1121 | GAUCCUGGUA | 369 | 1099-1121 | AACCUGCUAA | 3849 |
| | AAUACCAGG | | | UUCUUAGCAG | | | GAAUACCAGG | |
| | AUC | | | GUU | | | AUC | |
| 367 | CUGCUAAGA | 1943 | 1102-1122 | UGAUCCUGGU | 370 | 1100-1122 | ACCUGCUAAG | 3850 |
| | AUACCAGGA | | | AUUCUUAGCA | | | AAUACCAGGA | |
| | UCA | | | GGU | | | UCA | |
| 368 | UGCUAAGAA | 1944 | 1103-1123 | AUGAUCCUGG | 371 | 1101-1123 | CCUGCUAAGA | 3851 |
| | UACCAGGAU | | | UAUUCUUAGC | | | AUACCAGGAU | |
| | CAU | | | AGG | | | CAG | |
| 369 | GCUAAGAAU | 1945 | 1104-1124 | ACUGAUCCUG | 372 | 1102-1124 | CUGCUAAGAA | 3852 |
| | ACCAGGAUC | | | GUAUUCUUAG | | | UACCAGGAUC | |
| | AGU | | | CAG | | | AGU | |
| 370 | CUAAGAAUA | 1946 | 1105-1125 | UACUGAUCCU | 373 | 1103-1125 | UGCUAAGAAU | 3853 |
| | CCAGGAUCA | | | GGUAUUCUUA | | | ACCAGGAUCA | |
| | GUA | | | GCA | | | GUA | |
| 371 | UAAGAAUAC | 1947 | 1106-1126 | AUACUGAUCC | 374 | 1104-1126 | GCUAAGAAUA | 3854 |
| | CAGGAUCAG | | | UGGUAUUCUU | | | CCAGGAUCAG | |
| | UAU | | | AGC | | | UAU | |
| 372 | AAGAAUACC | 1948 | 1107-1127 | GAUACUGAUC | 375 | 1105-1127 | CUAAGAAUAC | 3855 |
| | AGGAUCAGU | | | CUGGUAUUCU | | | CAGGAUCAGU | |
| | AUC | | | UAG | | | AUC | |
| 373 | AGAAUACCA | 1949 | 1108-1128 | AGAUACUGAU | 376 | 1106-1128 | UAAGAAUACC | 3856 |
| | GGAUCAGUA | | | CCUGGUAUUC | | | AGGAUCAGUA | |
| | UCU | | | UUA | | | UCG | |
| 374 | GAAUACCAG | 1950 | 1109-1129 | UCGAUACUGA | 377 | 1107-1129 | AAGAAUACCA | 3857 |
| | GAUCAGUAU | | | UCCUGGUAUU | | | GGAUCAGUAU | |
| | CGA | | | CUU | | | CGA | |
| 375 | AAUACCAGG | 1951 | 1110-1130 | AUCGAUACUG | 378 | 1108-1130 | AGAAUACCAG | 3858 |
| | AUCAGUAUC | | | AUCCUGGUAU | | | GAUCAGUAUC | |
| | GAU | | | UCU | | | GAG | |
| 376 | AUACCAGGA | 1952 | 1111-1131 | UCUCGAUACU | 379 | 1109-1131 | GAAUACCAGG | 3859 |
| | UCAGUAUCG | | | GAUCCUGGUA | | | AUCAGUAUCG | |
| | AGA | | | UUC | | | AGA | |
| 377 | UACCAGGAU | 1953 | 1112-1132 | AUCUCGAUAC | 380 | 1110-1132 | AAUACCAGGA | 3860 |
| | CAGUAUCGA | | | UGAUCCUGGU | | | UCAGUAUCGA | |
| | GAU | | | AUU | | | GAG | |
| 378 | ACCAGGAUC | 1954 | 1113-1133 | UCUCUCGAUA | 381 | 1111-1133 | AUACCAGGAU | 3861 |
| | AGUAUCGAG | | | CUGAUCCUGG | | | CAGUAUCGAG | |
| | AGA | | | UAU | | | AGA | |
| 379 | CCAGGAUCA | 1955 | 1114-1134 | AUCUCUCGAU | 382 | 1112-1134 | UACCAGGAUC | 3862 |
| | GUAUCGAGA | | | ACUGAUCCUG | | | AGUAUCGAGA | |
| | GAU | | | GUA | | | GAG | |
| 380 | CAGGAUCAG | 1956 | 1115-1135 | ACUCUCUCGA | 383 | 1113-1135 | ACCAGGAUCA | 3863 |
| | UAUCGAGAG | | | UACUGAUCCU | | | GUAUCGAGAG | |
| | AGU | | | GGU | | | AGG | |
| 381 | AGGAUCAGU | 1957 | 1116-1136 | GCCUCUCUCG | 384 | 1114-1136 | CCAGGAUCAG | 3864 |
| | AUCGAGAGA | | | AUACUGAUCC | | | UAUCGAGAGA | |
| | GGC | | | UGG | | | GGC | |
| 382 | CCUGGCAGA | 1958 | 1143-1163 | GGAAAGCGCC | 385 | 1141-1163 | GGCCUGGCAG | 3865 |
| | AGGGCGCUU | | | CUUCUGCCAG | | | AAGGGCGCUU | |
| | UCC | | | GCC | | | UCC | |
| 383 | AGAAGGGCG | 1959 | 1149-1169 | GUGGGUGGAA | 386 | 1147-1169 | GCAGAAGGGC | 3866 |
| | CUUUCCACC | | | AGCGCCCUUC | | | GCUUUCCACC | |
| | CAC | | | UGC | | | CAC | |
| 384 | GAAGGGCGC | 1960 | 1150-1170 | AGUGGGUGGA | 387 | 1148-1170 | CAGAAGGGCG | 3867 |
| | UUUCCACCC | | | AAGCGCCCUU | | | CUUUCCACCC | |
| | ACU | | | CUG | | | ACU | |
| 385 | CUGUUUAGU | 1961 | 1177-1197 | UCUCGGUGGU | 388 | 1175-1197 | CCCUGUUUAG | 3868 |
| | CCACCACCG | | | GGACUAAACA | | | UCCACCACCG | |
| | AGA | | | GGG | | | AGA | |
| 386 | UGUUUAGUC | 1962 | 1178-1198 | GUCUCGGUGG | 389 | 1176-1198 | CCUGUUUAGU | 3869 |
| | CACCACCGA | | | UGGACUAAAC | | | CCACCACCGA | |
| | GAC | | | AGG | | | GAC | |
| 387 | GUUUAGUCC | 1963 | 1179-1199 | UGUCUCGGUG | 390 | 1177-1199 | CUGUUUAGUC | 3870 |
| | ACCACCGAG | | | GUGGACUAAA | | | CACCACCGAG | |
| | ACA | | | CAG | | | ACA | |
| 388 | UUUAGUCCA | 1964 | 1180-1200 | AUGUCUCGGU | 391 | 1178-1200 | UGUUUAGUCC | 3871 |
| | CCACCGAGA | | | GGUGGACUAA | | | ACCACCGAGA | |
| | CAU | | | ACA | | | CAU | |
| 389 | UUAGUCCAC | 1965 | 1181-1201 | GAUGUCUCGG | 392 | 1179-1201 | GUUUAGUCCA | 3872 |
| | CACCGAGAC | | | UGGUGGACUA | | | CCACCGAGAC | |
| | AUC | | | AAC | | | AUC | |
| 390 | UAGUCCACC | 1966 | 1182-1202 | UGAUGUCUCG | 393 | 1180-1202 | UUUAGUCCAC | 3873 |
| | ACCGAGACA | | | GUGGUGGACU | | | CACCGAGACA | |
| | UCA | | | AAA | | | UCA | |
| 391 | AGUCCACCA | 1967 | 1183-1203 | GUGAUGUCUC | 394 | 1181-1203 | UUAGUCCACC | 3874 |
| | CCGAGACAU | | | GGUGGUGGAC | | | ACCGAGACAU | |
| | CAC | | | UAA | | | CAC | |
| 392 | GUCCACCAC | 1968 | 1184-1204 | AGUGAUGUCU | 395 | 1182-1204 | UAGUCCACCA | 3875 |
| | CGAGACAUC | | | CGGUGGUGGA | | | CCGAGACAUC | |
| | ACU | | | CUA | | | ACU | |
| 393 | CCACCACCG | 1969 | 1186-1206 | AAAGUGAUGU | 396 | 1184-1206 | GUCCACCACC | 3876 |
| | AGACAUCAC | | | CUCGGUGGUG | | | GAGACAUCAC | |
| | UUU | | | GAC | | | UUG | |
| 394 | CACCACCGA | 1970 | 1187-1207 | ACAAGUGAUG | 397 | 1185-1207 | UCCACCACCG | 3877 |
| | GACAUCACU | | | UCUCGGUGGU | | | AGACAUCACU | |
| | UGU | | | GGA | | | UGG | |
| 395 | ACCACCGAG | 1971 | 1188-1208 | UCCAAGUGAU | 398 | 1186-1208 | CCACCACCGA | 3878 |
| | ACAUCACUU | | | GUCUCGGUGG | | | GACAUCACUU | |
| | GGA | | | UGG | | | GGA | |
| 396 | CCACCGAGA | 1972 | 1189-1209 | GUCCAAGUGA | 399 | 1187-1209 | CACCACCGAG | 3879 |
| | CAUCACUUG | | | UGUCUCGGUG | | | ACAUCACUUG | |
| | GAC | | | GUG | | | GAC | |
| 397 | UGCUGCGGU | 1973 | 1283-1303 | ACAUUGGAUU | 400 | 1281-1303 | GGUGCUGCGG | 3880 |
| | UGAAUCCAA | | | CAACCGCAGC | | | UUGAAUCCAA | |
| | UGU | | | ACC | | | UGG | |
| 398 | AGCCUCCCG | 1974 | 1310-1330 | AGAAAUCCAU | 401 | 1308-1330 | GGAGCCUCCC | 3881 |
| | CCAUGGAUU | | | GGCGGGAGGC | | | GCCAUGGAUU | |
| | UCU | | | UCC | | | UCU | |
| 399 | GCCUCCCGC | 1975 | 1311-1331 | GAGAAAUCCA | 402 | 1309-1331 | GAGCCUCCCG | 3882 |
| | CAUGGAUUU | | | UGGCGGGAGG | | | CCAUGGAUUU | |
| | CUC | | | CUC | | | CUC | |
| 400 | CUCCCGCCA | 1976 | 1313-1333 | UAGAGAAAUC | 403 | 1311-1333 | GCCUCCCGCC | 3883 |
| | UGGAUUUCU | | | CAUGGCGGGA | | | AUGGAUUUCU | |
| | CUA | | | GGC | | | CUA | |
| 401 | UCCCGCCAU | 1977 | 1314-1334 | AUAGAGAAAU | 404 | 1312-1334 | CCUCCCGCCA | 3884 |
| | GGAUUUCUC | | | CCAUGGCGGG | | | UGGAUUUCUC | |
| | UAU | | | AGG | | | UAG | |
| 402 | CCCGCCAUG | 1978 | 1315-1335 | ACUAGAGAAA | 405 | 1313-1335 | CUCCCGCCAU | 3885 |
| | GAUUUCUCU | | | UCCAUGGCGG | | | GGAUUUCUCU | |
| | AGU | | | GAG | | | AGG | |
| 403 | CGCCAUGGA | 1979 | 1317-1337 | AUCCUAGAGA | 406 | 1315-1337 | CCCGCCAUGG | 3886 |
| | UUUCUCUAG | | | AAUCCAUGGC | | | AUUUCUCUAG | |
| | GAU | | | GGG | | | GAG | |
| 404 | UAGGAGACU | 1980 | 1332-1352 | GCCAGCUCUC | 407 | 1330-1352 | UCUAGGAGAC | 3887 |
| | UAGAGAGCU | | | UAAGUCUCCU | | | UUAGAGAGCU | |
| | GGC | | | AGA | | | GGC | |
| 405 | AGGAGACUU | 1981 | 1333-1353 | UGCCAGCUCU | 408 | 1331-1353 | CUAGGAGACU | 3888 |
| | AGAGAGCUG | | | CUAAGUCUCC | | | UAGAGAGCUG | |
| | GCA | | | UAG | | | GCA | |
| 406 | GGAACACGU | 1982 | 1334-1354 | GCGGUGGGCU | 409 | 1332-1354 | AGGGAACACG | 3889 |
| | CUAGCCCAC | | | AGACGUGUUC | | | UCUAGCCCAC | |
| | CGC | | | CCU | | | CGC | |
| 407 | CCUAUGCAA | 1983 | 1396-1416 | UUGCAGUAAC | 410 | 1394-1416 | GCCCUAUGCA | 3890 |
| | AGGUUACUG | | | CUUUGCAUAG | | | AAGGUUACUG | |
| | CAA | | | GGC | | | CAA | |
| 408 | CUAUGCAAA | 1984 | 1397-1417 | GUUGCAGUAA | 411 | 1395-1417 | CCCUAUGCAA | 3891 |
| | GGUUACUGC | | | CCUUUGCAUA | | | AGGUUACUGC | |
| | AAC | | | GGG | | | AAC | |
| 409 | UAUGCAAAG | 1985 | 1398-1418 | GGUUGCAGUA | 412 | 1396-1418 | UAUGCAAAGG | 3892 |
| | GUUACUGCA | | | ACCUUUGCAU | | | UUACUGCAAC | |
| | ACC | | | AGG | | | C | |
| 410 | UGCAAAGGU | 1986 | 1400-1420 | AUGGUUGCAG | 413 | 1398-1420 | UAUGCAAAGG | 3893 |
| | UACUGCAAC | | | UAACCUUUGC | | | UUACUGCAAC | |
| | CAU | | | AUA | | | CAU | |
| 411 | GCAAAGGUU | 1987 | 1411-1421 | AAUGGUUGCA | 414 | 1399-1421 | AUGCAAAGGU | 3894 |
| | ACUGCAACC | | | GUAACCUUUG | | | UACUGCAACC | |
| | AUU | | | CAU | | | AUU | |
| 412 | CAAAGGUUA | 1988 | 1402-1422 | GAAUGGUUGC | 415 | 1400-1422 | UGCAAAGGUU | 3895 |
| | CUGCAACCA | | | AGUAACCUUU | | | ACUGCAACCA | |
| | UUC | | | GCA | | | UUC | |
| 413 | AAAGGUUAC | 1989 | 1403-1423 | GGAAUGGUUG | 416 | 1401-1423 | GCAAAGGUUA | 3896 |
| | UGCAACCAU | | | CAGUAACCUU | | | CUGCAACCAU | |
| | UCC | | | UGC | | | UCC | |
| 414 | AAGGUUACU | 1990 | 1404-1424 | UGGAAUGGUU | 417 | 1402-1424 | CAAAGGUUAC | 3897 |
| | GCAACCAUU | | | GCAGUAACCU | | | UGCAACCAUU | |
| | CCA | | | UUG | | | CCA | |
| 415 | AGGUUACUG | 1991 | 1405-1425 | AUGGAAUGGU | 418 | 1403-1425 | AAAGGUUACU | 3898 |
| | CAACCAUUC | | | UGCAGUAACC | | | GCAACCAUUC | |
| | CAU | | | UUU | | | CAG | |
| 416 | GGUUACUGC | 1992 | 1406-1426 | GCUGGAAUGG | 419 | 1404-1426 | AAGGUUACUG | 3899 |
| | AACCAUUCC | | | UUGCAGUAAC | | | CAACCAUUCC | |
| | AGC | | | CUU | | | AGC | |
| 417 | UACUGCAAC | 1993 | 1409-1429 | AUGGCUGGAA | 420 | 1407-1429 | GUUACUGCAA | 3900 |
| | CAUUCCAGC | | | UGGUUGCAGU | | | CCAUUCCAGC | |
| | CAU | | | AAC | | | CA | |
| 418 | CGGUCAAGU | 1994 | 1502-1522 | AGCAUGACUU | 421 | 1500-1522 | CCCGGUCAAG | 3901 |
| | CCAAGUCAU | | | GGACUUGACC | | | UCCAAGUCAU | |
| | GCU | | | GGG | | | GC | |
| 419 | GGUCAAGUC | 1995 | 1503-1523 | UCGCAUGACU | 422 | 1501-1523 | CCGGUCAAGU | 3902 |
| | CAAGUCAUG | | | UGGACUUGAC | | | CCAAGUCAUG | |
| | CGA | | | CGG | | | CGA | |
| 420 | GUCAAGUCC | 1996 | 1504-1524 | AUCGCAUGAC | 423 | 1502-1524 | CGGUCAAGUC | 3903 |
| | AAGUCAUGC | | | UUGGACUUGA | | | CAAGUCAUGC | |
| | GAU | | | CCG | | | GAG | |
| 421 | UCAAGUCCA | 1997 | 1505-1525 | ACUCGCAUGA | 424 | 1503-1525 | GGUCAAGUCC | 3904 |
| | AGUCAUGCG | | | CUUGGACUUG | | | AAGUCAUGCG | |
| | AGU | | | ACC | | | AGU | |
| 422 | CAAGUCCAA | 1998 | 1506-1526 | AACUCGCAUG | 425 | 1504-1526 | GUCAAGUCCA | 3905 |
| | GUCAUGCGA | | | ACUUGGACUU | | | AGUCAUGCGA | |
| | GUU | | | GAC | | | GUU | |
| 423 | AAGUCCAAG | 1999 | 1507-1527 | GAACUCGCAU | 426 | 1505-1527 | UCAAGUCCAA | 3906 |
| | UCAUGCGAG | | | GACUUGGACU | | | GUCAUGCGAG | |
| | UUC | | | UGA | | | UUC | |
| 424 | AGUCCAAGU | 2000 | 1508-1528 | AGAACUCGCA | 427 | 1506-1528 | CAAGUCCAAG | 3907 |
| | CAUGCGAGU | | | UGACUUGGAC | | | UCAUGCGAGU | |
| | UCU | | | UUG | | | UCU | |
| 425 | GUCCAAGUC | 2001 | 1509-1529 | AAGAACUCGC | 428 | 1507-1529 | AAGUCCAAGU | 3908 |
| | AUGCGAGUU | | | AUGACUUGGA | | | CAUGCGAGUU | |
| | CUU | | | CUU | | | CUG | |
| 426 | UCCAAGUCA | 2002 | 1510-1530 | GCAGAACUCG | 429 | 1508-1530 | AGUCCAAGUC | 3909 |
| | UGCGAGUUC | | | CAUGACUUGG | | | AUGCGAGUUC | |
| | UGC | | | ACU | | | UGC | |
| 427 | CCAAGUCAU | 2003 | 1511-1531 | AGCAGAACUC | 430 | 1509-1531 | GUCCAAGUCA | 3910 |
| | GCGAGUUCU | | | GCAUGACUUG | | | UGCGAGUUCU | |
| | GCU | | | GAC | | | GCG | |
| 428 | CAAGUCAUG | 2004 | 1512-1532 | ACGCAGAACU | 431 | 1510-1532 | UCCAAGUCAU | 3911 |
| | CGAGUUCUG | | | CGCAUGACUU | | | GCGAGUUCUG | |
| | CGU | | | GGA | | | CGG | |
| 429 | AGUCAUGCG | 2005 | 1514-1534 | UGCCGCAGAA | 432 | 1512-1534 | CAAGUCAUGC | 3912 |
| | AGUUCUGCG | | | CUCGCAUGAC | | | GAGUUCUGCG | |
| | GCA | | | UUG | | | GCA | |
| 430 | GUCAUGCGA | 2006 | 1515-1535 | UUGCCGCAGA | 433 | 1513-1535 | AAGUCAUGCG | 3913 |
| | GUUCUGCGG | | | ACUCGCAUGA | | | AGUUCUGCGG | |
| | CAA | | | CUU | | | CAA | |
| 431 | UCAUGCGAG | 2007 | 1516-1536 | AUUGCCGCAG | 434 | 1514-1536 | AGUCAUGCGA | 3914 |
| | UUCUGCGGC | | | AACUCGCAUG | | | GUUCUGCGGC | |
| | AAU | | | ACU | | | AAG | |
| 432 | GCGAGUUCU | 2008 | 1520-1540 | ACGUCUUGCC | 435 | 1518-1540 | AUGCGAGUUC | 3915 |
| | GCGGCAAGA | | | GCAGAACUCG | | | UGCGGCAAGA | |
| | CGU | | | CAU | | | CGU | |
| 433 | CGAGUUCUG | 2009 | 1521-1541 | AACGUCUUGC | 436 | 1519-1541 | UGCGAGUUCU | 3916 |
| | CGGCAAGAC | | | CGCAGAACUC | | | GCGGCAAGAC | |
| | GUU | | | GCA | | | GUU | |
| 434 | GAGUUCUGC | 2010 | 1522-1542 | GAACGUCUUG | 437 | 1520-1542 | GCGAGUUCUG | 3917 |
| | GGCAAGACG | | | CCGCAGAACU | | | CGGCAAGACG | |
| | UUC | | | CGC | | | UUC | |
| 435 | AGUUCUGCG | 2011 | 1523-1543 | UGAACGUCUU | 438 | 1521-1543 | CGAGUUCUGC | 3918 |
| | GCAAGACGU | | | GCCGCAGAAC | | | GGCAAGACGU | |
| | UCA | | | UCG | | | UCA | |
| 436 | GUUCUGCGG | 2012 | 1524-1544 | UUGAACGUCU | 439 | 1522-1544 | GAGUUCUGCG | 3919 |
| | CAAGACGUU | | | UGCCGCAGAA | | | GCAAGACGUU | |
| | CAA | | | CUC | | | CAA | |
| 437 | UUCUGCGGC | 2013 | 1525-1545 | UUUGAACGUC | 440 | 1523-1545 | AGUUCUGCGG | 3920 |
| | AAGACGUUC | | | UUGCCGCAGA | | | CAAGACGUUC | |
| | AAA | | | ACU | | | AAA | |
| 438 | UCUGCGGCA | 2014 | 1526-1546 | AUUUGAACGU | 441 | 1524-1546 | GUUCUGCGGC | 3921 |
| | AGACGUUCA | | | CUUGCCGCAG | | | AAGACGUUCA | |
| | AAU | | | AAC | | | AAU | |
| 439 | CUGCGGCAA | 2015 | 1527-1547 | AAUUUGAACG | 442 | 1525-1547 | UUCUGCGGCA | 3922 |
| | GACGUUCAA | | | UCUUGCCGCA | | | AGACGUUCAA | |
| | AUU | | | GAA | | | AUU | |
| 440 | UGCGGCAAG | 2016 | 1528-1548 | AAAUUUGAAC | 443 | 1526-1548 | UCUGCGGCAA | 3923 |
| | ACGUUCAAA | | | GUCUUGCCGC | | | GACGUUCAAA | |
| | UUU | | | AGA | | | UUU | |
| 441 | GCGGCAAGA | 2017 | 1529-1549 | GAAAUUUGAA | 444 | 1527-1549 | CUGCGGCAAG | 3924 |
| | CGUUCAAAU | | | CGUCUUGCCG | | | ACGUUCAAAU | |
| | UUC | | | CAG | | | UUC | |
| 442 | CAUGACGGU | 2018 | 1683-1703 | UCGUCGGACU | 445 | 1681-1703 | CCCAUGACGG | 3925 |
| | CAAGUCCGA | | | UGACCGUCAU | | | UCAAGUCCGA | |
| | CGA | | | GGG | | | CGA | |
| 443 | CAAGUCCGU | 2019 | 1773-1793 | AACUUGGCCA | 446 | 1771-1793 | CUCAAGUCCG | 3926 |
| | GGUGGCCAA | | | CCACGGACUU | | | UGGUGGCCAA | |
| | GUU | | | GAG | | | GUU | |
| 444 | AAGUCCGUG | 2020 | 1774-1794 | GAACUUGGCC | 447 | 1772-1794 | UCAAGUCCGU | 3927 |
| | GUGGCCAAG | | | ACCACGGACU | | | GGUGGCCAAG | |
| | UUC | | | UGA | | | UUC | |
| 445 | UCCGUGGUG | 2021 | 1777-1797 | AUUGAACUUG | 448 | 1775-1797 | AGUCCGUGGU | 3928 |
| | GCCAAGUUC | | | GCCACCACGG | | | GGCCAAGUUC | |
| | AAU | | | ACU | | | AAG | |
| 446 | AGAGGAGGA | 2022 | 1845-1865 | UCUUCCUCGU | 449 | 1843-1865 | GAAGAGGAGG | 3929 |
| | CGACGAGGA | | | CGUCCUCCUC | | | ACGACGAGGA | |
| | AGA | | | UUC | | | AGA | |
| 447 | GAGGAGGAC | 2023 | 1846-1866 | AUCUUCCUCG | 450 | 1844-1866 | AAGAGGAGGA | 3930 |
| | GACGAGGAA | | | UCGUCCUCCU | | | CGACGAGGAA | |
| | GAU | | | CUU | | | GAG | |
| 448 | GGAGCUGAC | 2024 | 1893-1913 | AUCUCGCUCU | 451 | 1891-1913 | GAGGAGCUGA | 3931 |
| | GGAGAGCGA | | | CCGUCAGCUC | | | CGGAGAGCGA | |
| | GAU | | | CUC | | | GAG | |
| 449 | AGCUGACGG | 2025 | 1895-1915 | ACCUCUCGCU | 452 | 1893-1915 | GGAGCUGACG | 3932 |
| | AGAGCGAGA | | | CUCCGUCAGC | | | GAGAGCGAGA | |
| | GGU | | | UCC | | | GGG | |
| 450 | GCUGACGGA | 2026 | 1896-1916 | ACCCUCUCGC | 453 | 1894-1916 | GAGCUGACGG | 3933 |
| | GAGCGAGAG | | | UCUCCGUCAG | | | AGAGCGAGAG | |
| | GGU | | | CUC | | | GGU | |
| 451 | CUGACGGAG | 2027 | 1897-1917 | AACCCUCUCG | 454 | 1895-1917 | AGCUGACGGA | 3934 |
| | AGCGAGAGG | | | CUCUCCGUCA | | | GAGCGAGAGG | |
| | GUU | | | GCU | | | GUG | |
| 452 | CAUGCAGCA | 2028 | 2046-2066 | GCCUCGCUGA | 455 | 2044-2066 | UCCAUGCAGC | 3935 |
| | CUUCAGCGA | | | AGUGCUGCAU | | | ACUUCAGCGA | |
| | GGC | | | GGA | | | GGC | |
| 453 | UGCAGCACU | 2029 | 2048-2068 | AGGCCUCGCU | 456 | 2046-2068 | CAUGCAGCAC | 3936 |
| | UCAGCGAGG | | | GAAGUGCUGC | | | UUCAGCGAGG | |
| | CCU | | | AUG | | | CCU | |
| 454 | UCAGCGAGG | 2030 | 2057-2077 | ACUGGUGGAA | 457 | 2055-2077 | CUUCAGCGAG | 3937 |
| | CCUUCCACC | | | GGCCUCGCUG | | | GCCUUCCACC | |
| | AGU | | | AAG | | | AGG | |
| 455 | ACCGCAUAG | 2031 | 2168-2188 | AAGUGCCAUC | 458 | 2166-2188 | GGACCGCAUA | 3938 |
| | ACGAUGGCA | | | GUCUAUGCGG | | | GACGAUGGCA | |
| | CUU | | | UCC | | | CU | |
| 456 | CGCAUAGAC | 2032 | 2170-2190 | AACAGUGCCA | 459 | 2168-2190 | ACCGCAUAGA | 3939 |
| | GAUGGCACU | | | UCGUCUAUGC | | | CGAUGGCACU | |
| | GUU | | | GGU | | | GUU | |
| 457 | CAUAGACGA | 2033 | 2172-2192 | UUAACAGUGC | 460 | 2170-2192 | CGCAUAGACG | 3940 |
| | UGGCACUGU | | | CAUCGUCUAU | | | AUGGCACUGU | |
| | UAA | | | GCG | | | UAA | |
| 458 | AGGCAGCUC | 2034 | 2383-2403 | GAAGGGAUCU | 461 | 2381-2403 | CCAGGCAGCU | 3941 |
| | AAAGAUCCC | | | UUGAGCUGCC | | | CAAAGAUCCC | |
| | UUC | | | UGG | | | UUC | |
| 459 | GGCAGCUCA | 2035 | 2384-2404 | GGAAGGGAUC | 462 | 2382-2404 | CAGGCAGCUC | 3942 |
| | AAGAUCCCU | | | UUUGAGCUGC | | | AAAGAUCCCU | |
| | UCC | | | CUG | | | UCC | |
| 460 | GCAGCUCAA | 2036 | 2385-2405 | AGGAAGGGAU | 463 | 2383-2405 | AGGCAGCUCA | 3943 |
| | AGAUCCCUU | | | CUUUGAGCUG | | | AAGAUCCCUU | |
| | CCU | | | CCU | | | CCU | |
| 461 | UCGGAGACU | 2037 | 2411-2431 | GCGAUUGUCU | 464 | 2409-2431 | CUUCGGAGAC | 3944 |
| | CCAGACAAU | | | GGAGUCUCCG | | | UCCAGACAAU | |
| | CGC | | | AAG | | | CGC | |
| 462 | CGGAGACUC | 2038 | 2412-2432 | GGCGAUUGUC | 465 | 2410-2432 | UUCGGAGACU | 3945 |
| | CAGACAAUC | | | UGGAGUCUCC | | | CCAGACAAUC | |
| | GCC | | | GAA | | | GCC | |
| 463 | GGAGACUCC | 2039 | 2413-2433 | AGGCGAUUGU | 466 | 2411-2433 | UCGGAGACUC | 3946 |
| | AGACAAUCG | | | CUGGAGUCUC | | | CAGACAAUCG | |
| | CCU | | | CGA | | | CCU | |
| 464 | GAGACUCCA | 2040 | 2414-2434 | AAGGCGAUUG | 467 | 2412-2434 | CGGAGACUCC | 3947 |
| | GACAAUCGC | | | UCUGGAGUCU | | | AGACAAUCGC | |
| | CUU | | | CCG | | | CUU | |
| 465 | AGACUCCAG | 2041 | 2415-2435 | AAAGGCGAUU | 468 | 2413-2435 | GGAGACUCCA | 3948 |
| | ACAAUCGCC | | | GUCUGGAGUC | | | GACAAUCGCC | |
| | UUU | | | UCC | | | UUU | |
| 466 | GACUCCAGA | 2042 | 2416-2436 | AAAAGGCGAU | 469 | 2414-2436 | GAGACUCCAG | 3949 |
| | CAAUCGCCU | | | UGUCUGGAGU | | | ACAAUCGCCU | |
| | UUU | | | CUC | | | UUU | |
| 467 | ACUCCAGAC | 2043 | 2417-2437 | AAAAAGGCGA | 470 | 2415-2437 | AGACUCCAGA | 3950 |
| | AAUCGCCUU | | | UUGUCUGGAG | | | CAAUCGCCUU | |
| | UUU | | | UCU | | | UUG | |
| 468 | CUCCAGACA | 2044 | 2418-2438 | GCAAAAGGCG | 471 | 2416-2438 | GACUCCAGAC | 3951 |
| | AUCGCCUUU | | | AUUGUCUGGA | | | AAUCGCCUUU | |
| | UGC | | | GUC | | | UGC | |
| 469 | CCAGACAAU | 2045 | 2420-2440 | AGGCAAAAGG | 472 | 2418-2440 | CUCCAGACAA | 3952 |
| | CGCCUUUUG | | | CGAUUGUCUG | | | UCGCCUUUUG | |
| | CCU | | | GAG | | | CCU | |
| 470 | CAGACAAUC | 2046 | 2421-2441 | GAGGCAAAAG | 473 | 2419-2441 | UCCAGACAAU | 3953 |
| | GCCUUUUGC | | | GCGAUUGUCU | | | CGCCUUUUGC | |
| | CUC | | | GGA | | | CUC | |
| 471 | GACGCAGCG | 2047 | 2600-2620 | ACUCACAAGU | 474 | 2598-2620 | CAGACGCAGC | 3954 |
| | ACACUUGUG | | | GUCGCUGCGU | | | GACACUUGUG | |
| | AGU | | | CUG | | | AGU | |
| 472 | CGCAGCGAC | 2048 | 2602-2622 | GUACUCACAA | 475 | 2600-2622 | GACGCAGCGA | 3955 |
| | ACUUGUGAG | | | GUGUCGCUGC | | | CACUUGUGAG | |
| | UAC | | | GUC | | | UAC | |
| 473 | GCAGCGACA | 2049 | 2603-2623 | AGUACUCACA | 476 | 2601-2623 | ACGCAGCGAC | 3956 |
| | CUUGUGAGU | | | AGUGUCGCUG | | | ACUUGUGAGU | |
| | ACU | | | CGU | | | ACU | |
| 474 | CAGCGACAC | 2050 | 2604-2624 | AAGUACUCAC | 477 | 2602-2624 | CGCAGCGACA | 3957 |
| | UUGUGAGUA | | | AAGUGUCGCU | | | CUUGUGAGUA | |
| | CUU | | | GCG | | | CUG | |
| 475 | GACACUUGU | 2051 | 2608-2628 | ACCACAGUAC | 478 | 2606-2628 | GCGACACUUG | 3958 |
| | GAGUACUGU | | | UCACAAGUGU | | | UGAGUACUGU | |
| | GGU | | | CGC | | | GGG | |
| 476 | ACACUUGUG | 2052 | 2609-2629 | UCCCACAGUA | 479 | 2607-2629 | CGACACUUGU | 3959 |
| | AGUACUGUG | | | CUCACAAGUG | | | GAGUACUGUG | |
| | GGA | | | UCG | | | GGA | |
| 477 | CACUUGUGA | 2053 | 2610-2630 | UUCCCACAGU | 480 | 2608-2630 | GACACUUGUG | 3960 |
| | GUACUGUGG | | | ACUCACAAGU | | | AGUACUGUGG | |
| | GAA | | | GUC | | | GAA | |
| 478 | ACUUGUGAG | 2054 | 2611-2631 | UUUCCCACAG | 481 | 2609-2631 | ACACUUGUGA | 3961 |
| | UACUGUGGG | | | UACUCACAAG | | | GUACUGUGGG | |
| | AAA | | | UGU | | | AA | |
| 479 | CUUGUGAGU | 2055 | 2612-2632 | AUUUCCCACA | 482 | 2610-2632 | CACUUGUGAG | 3962 |
| | ACUGUGGGA | | | GUACUCACAA | | | UACUGUGGGA | |
| | AAU | | | GUG | | | AAG | |
| 480 | UUGUGAGUA | 2056 | 2613-2633 | ACUUUCCCAC | 483 | 2611-2633 | ACUUGUGAGU | 3963 |
| | CUGUGGGAA | | | AGUACUCACA | | | ACUGUGGGAA | |
| | AGU | | | AGU | | | AGU | |
| 481 | UGUGAGUAC | 2057 | 2614-2634 | GACUUUCCCA | 484 | 2612-2634 | CUUGUGAGUA | 3964 |
| | UGUGGGAAA | | | CAGUACUCAC | | | CUGUGGGAAA | |
| | GUC | | | AAG | | | GUC | |
| 482 | GUGAGUACU | 2058 | 2615-2635 | AGACUUUCCC | 485 | 2613-2635 | UUGUGAGUAC | 3965 |
| | GUGGGAAAG | | | ACAGUACUCA | | | UGUGGGAAAG | |
| | UCU | | | CAA | | | UCU | |
| 483 | GUCUUCAAG | 2059 | 2632-2652 | AUUGCUACAG | 486 | 2630-2652 | AAGUCUUCAA | 3966 |
| | AACUGUAGC | | | UUCUUGAAGA | | | GAACUGUAGC | |
| | AAU | | | CUU | | | AAU | |
| 484 | UCUUCAAGA | 2060 | 2633-2653 | GAUUGCUACA | 487 | 2631-2653 | AGUCUUCAAG | 3967 |
| | ACUGUAGCA | | | GUUCUUGAAG | | | AACUGUAGCA | |
| | AUC | | | ACU | | | AUC | |
| 485 | CUUCAAGAA | 2061 | 2634-2654 | AGAUUGCUAC | 488 | 2632-2654 | GUCUUCAAGA | 3968 |
| | CUGUAGCAA | | | AGUUCUUGAA | | | ACUGUAGCAA | |
| | UCU | | | GAC | | | UCU | |
| 486 | UUCAAGAAC | 2062 | 2635-2655 | GAGAUUGCUA | 489 | 2633-2655 | UCUUCAAGAA | 3969 |
| | UGUAGCAAU | | | CAGUUCUUGA | | | CUGUAGCAAU | |
| | CUC | | | AGA | | | CUC | |
| 487 | UCAAGAACU | 2063 | 2636-2656 | UGAGAUUGCU | 490 | 2634-2656 | CUUCAAGAAC | 3970 |
| | GUAGCAAUC | | | ACAGUUCUUG | | | UGUAGCAAUC | |
| | UCA | | | AAG | | | UCA | |
| 488 | CAAGAACUG | 2064 | 2637-2657 | GUGAGAUUGC | 491 | 2635-2657 | UUCAAGAACU | 3971 |
| | UAGCAAUCU | | | UACAGUUCUU | | | GUAGCAAUCU | |
| | CAC | | | GAA | | | CAC | |
| 489 | AAGAACUGU | 2065 | 2638-2658 | AGUGAGAUUG | 492 | 2636-2658 | UCAAGAACUG | 3972 |
| | AGCAAUCUC | | | CUACAGUUCU | | | UAGCAAUCUC | |
| | ACU | | | UGA | | | ACU | |
| 490 | GAGAAGCCA | 2066 | 2667-2687 | AUUUCGCCCG | 493 | 2665-2687 | AGGAGAAGCC | 3973 |
| | CACGGGCGA | | | UGUGGCUUCU | | | ACACGGGCGA | |
| | AAU | | | CCU | | | AAG | |
| 491 | AGCCACACG | 2067 | 2671-2691 | AGGCCUUUCG | 494 | 2669-2691 | GAAGCCACAC | 3974 |
| | GGCGAAAGG | | | CCCGUGUGGC | | | GGGCGAAAGG | |
| | CCU | | | UUC | | | CCU | |
| 492 | GCCACACGG | 2068 | 2672-2692 | AAGGCCUUUC | 495 | 2670-2692 | AAGCCACACG | 3975 |
| | GCGAAAGGC | | | GCCCGUGUGG | | | GGCGAAAGGC | |
| | CUU | | | CUU | | | CUU | |
| 493 | ACACGGGCG | 2069 | 2674-2695 | UAUAAGGCCU | 496 | 2673-2695 | CCACACGGGC | 3976 |
| | AAAGGCCUU | | | UUCGCCCGUG | | | GAAAGGCCUU | |
| | AUA | | | UGG | | | AUA | |
| 494 | CACGGGCGA | 2070 | 2676-2696 | UUAUAAGGCC | 497 | 2674-2696 | CACACGGGCG | 3977 |
| | AAGGCCUUA | | | UUUCGCCCGU | | | AAAGGCCUUA | |
| | UAA | | | GUG | | | UAA | |
| 495 | ACGGGCGAA | 2071 | 2677-2697 | UUUAUAAGGC | 498 | 2675-2697 | ACACGGGCGA | 3978 |
| | AGGCCUUAU | | | CUUUCGCCCG | | | AAGGCCUUAU | |
| | AAA | | | UGU | | | AAA | |
| 496 | CGGGCGAAA | 2072 | 2678-2698 | AUUUAUAAGG | 499 | 2676-2698 | CACGGGCGAA | 3979 |
| | GGCCUUAUA | | | CCUUUCGCCC | | | AGGCCUUAUA | |
| | AAU | | | GUG | | | AAU | |
| 497 | GGGCGAAAG | 2073 | 2679-2699 | AAUUUAUAAG | 500 | 2677-2699 | ACGGGCGAAA | 3980 |
| | GCCUUAUAA | | | GCCUUUCGCC | | | GGCCUUAUAA | |
| | AUU | | | CGU | | | AUG | |
| 498 | GGCGAAAGG | 2074 | 2680-2700 | GCAUUUAUAA | 501 | 2678-2700 | CGGGCGAAAG | 3981 |
| | CCUUAUAAA | | | GGCCUUUCGC | | | GCCUUAUAAA | |
| | UGC | | | CCG | | | UGC | |
| 499 | GCGAAAGGC | 2075 | 2681-2701 | AGCAUUUAUA | 502 | 2679-2701 | GGGCGAAAGG | 3982 |
| | CUUAUAAAU | | | AGGCCUUUCG | | | CCUUAUAAAU | |
| | GCU | | | CCC | | | GCG | |
| 500 | GAAAGGCCU | 2076 | 2683-2703 | AUCGCAUUUA | 503 | 2681-2703 | GCGAAAGGCC | 3983 |
| | UAUAAAUGC | | | UAAGGCCUUU | | | UUAUAAAUGC | |
| | GAU | | | CGC | | | GAG | |
| 501 | AAAGGCCUU | 2077 | 2684-2704 | GCUCGCAUUU | 504 | 2682-2704 | CGAAAGGCCU | 3984 |
| | AUAAAUGCG | | | AUAAGGCCUU | | | UAUAAAUGCG | |
| | AGC | | | UCG | | | AGG | |
| 502 | AAGGCCUUA | 2078 | 2685-2705 | AGCUCGCAUU | 505 | 2683-2705 | GAAAGGCCUU | 3985 |
| | UAAAUGCGA | | | UAUAAGGCCU | | | AUAAAUGCGA | |
| | GCU | | | UUC | | | GCU | |
| 503 | AGGCCUUAU | 2079 | 2686-2706 | AAGCUCGCAU | 506 | 2684-2706 | AAAGGCCUUA | 3986 |
| | AAAUGCGAG | | | UUAUAAGGCC | | | UAAAUGCGAG | |
| | CUU | | | UUU | | | CUG | |
| 504 | GGCCUUAUA | 2080 | 2687-2707 | ACAGCUCGCA | 507 | 2685-2707 | AAGGCCUUAU | 3987 |
| | AAUGCGAGC | | | UUUAUAAGGC | | | AAAUGCGAGC | |
| | UGU | | | CUU | | | UG | |
| 505 | GCCUUAUAA | 2081 | 2688-2708 | AACAGCUCGC | 508 | 2686-2708 | AGGCCUUAUA | 3988 |
| | AUGCGAGCU | | | AUUUAUAAGG | | | AAUGCGAGCU | |
| | GUU | | | CCU | | | GUG | |
| 506 | CCUUAUAAA | 2082 | 2689-2709 | GCACAGCUCG | 509 | 2687-2709 | GGCCUUAUAA | 3989 |
| | UGCGAGCUG | | | CAUUUAUAAG | | | AUGCGAGCUG | |
| | UGC | | | GCC | | | UGC | |
| 507 | CUUAUAAAU | 2083 | 2690-2710 | UGCACAGCUC | 510 | 2688-2710 | GCCUUAUAAA | 3990 |
| | GCGAGCUGU | | | GCAUUUAUAA | | | UGCGAGCUGU | |
| | GCA | | | GGC | | | GCA | |
| 508 | UUAUAAAUG | 2084 | 2691-2711 | UUGCACAGCU | 511 | 2689-2711 | GCCUUAUAAA | 3991 |
| | CGAGCUGUG | | | CGCAUUUAUA | | | UGCGAGCUGU | |
| | CAA | | | AGG | | | GCAA | |
| 509 | UAUAAAUGC | 2085 | 2692-2712 | GUUGCACAGC | 512 | 2690-2712 | CUUAUAAAUG | 3992 |
| | GAGCUGUGC | | | UCGCAUUUAU | | | CGAGCUGUGC | |
| | AAC | | | AAG | | | AACU | |
| 510 | AUAAAUGCG | 2086 | 2693-2713 | AGUUGCACAG | 513 | 2691-2713 | UUAUAAAUGC | 3993 |
| | AGCUGUGCA | | | CUCGCAUUUA | | | GAGCUGUGCA | |
| | ACU | | | UAA | | | ACU | |
| 511 | UAAAUGCGA | 2087 | 2694-2714 | UAGUUGCACA | 514 | 2692-2714 | UAUAAAUGCG | 3994 |
| | GCUGUGCAA | | | GCUCGCAUUU | | | AGCUGUGCAA | |
| | CUA | | | AUA | | | CUA | |
| 512 | AAAUGCGAG | 2088 | 2695-2715 | AUAGUUGCAC | 515 | 2693-2715 | AUAAAUGCGA | 3995 |
| | CUGUGCAAC | | | AGCUCGCAUU | | | GCUGUGCAAC | |
| | UAU | | | UAU | | | UAU | |
| 513 | AAUGCGAGC | 2089 | 2696-2716 | AAUAGUUGCA | 516 | 2694-2716 | UAAAUGCGAG | 3996 |
| | UGUGCAACU | | | CAGCUCGCAU | | | CUGUGCAACU | |
| | AUU | | | UUA | | | AUG | |
| 514 | AUGCGAGCU | 2090 | 2697-2717 | GCAUAGUUGC | 517 | 2695-2717 | AAAUGCGAGC | 3997 |
| | GUGCAACUA | | | ACAGCUCGCA | | | UGUGCAACUA | |
| | UGC | | | UUU | | | UGC | |
| 515 | GCGAGCUGU | 2091 | 2699-2719 | AGGCAUAGUU | 518 | 2697-2719 | AUGCGAGCUG | 3998 |
| | GCAACUAUG | | | GCACAGCUCG | | | UGCAACUAUG | |
| | CCU | | | CAU | | | CCU | |
| 516 | GAGCUGUGC | 2092 | 2701-2691 | ACAGGCAUAG | 519 | 2669-2691 | GCGAGCUGUG | 3999 |
| | AACUAUGCC | | | UUGCACAGCU | | | CAACUAUGCC | |
| | UGU | | | CGC | | | UGU | |
| 517 | GCUGUGCAA | 2093 | 2703-2693 | GCACAGGCAU | 520 | 2671-2693 | GAGCUGUGCA | 4000 |
| | CUAUGCCUG | | | AGUUGCACAG | | | ACUAUGCCUG | |
| | UGC | | | CUC | | | UGC | |
| 518 | GGGAAGGAC | 2094 | 2747-2767 | ACAUUUGUAA | 521 | 2745-2767 | UGGGGAAGGA | 4001 |
| | GUUUACAAA | | | ACGUCCUUCC | | | CGUUUACAAA | |
| | UGU | | | CCA | | | UGU | |
| 519 | GGAAGGACG | 2095 | 2748-2768 | AACAUUUGUA | 522 | 2746-2768 | GGGGAAGGAC | 4002 |
| | UUUACAAAU | | | AACGUCCUUC | | | GUUUACAAAU | |
| | GUU | | | CCC | | | GUG | |
| 520 | UUGUAAGAU | 2096 | 2773-2793 | ACGCUAAAAG | 523 | 2771-2793 | AUUUGUAAGA | 4003 |
| | GCCUUUUAG | | | GCAUCUUACA | | | UGCCUUUUAG | |
| | CGU | | | AAU | | | CGU | |
| 521 | UGUAAGAUG | 2097 | 2774-2794 | AACGCUAAAA | 524 | 2772-2794 | UUUGUAAGAU | 4004 |
| | CCUUUUAGC | | | GGCAUCUUAC | | | GCCUUUUAGC | |
| | GUU | | | AAA | | | GUG | |
| 522 | GUAAGAUGC | 2098 | 2775-2795 | ACACGCUAAA | 525 | 2773-2795 | UUGUAAGAUG | 3562 |
| | CUUUUAGCG | | | AGGCAUCUUA | | | CCUUUUAGCG | |
| | UGU | | | CAA | | | UGU | |
| 523 | UAAGAUGCC | 2099 | 2776-2796 | UACACGCUAA | 526 | 2774-2796 | UGUAAGAUGC | 4005 |
| | UUUUAGCGU | | | AAGGCAUCUU | | | CUUUUAGCGU | |
| | GUA | | | ACA | | | GUA | |
| 524 | AAGAUGCCU | 2100 | 2778-2798 | GUACACGCUA | 527 | 2776-2798 | GUAAGAUGCC | 4006 |
| | UUUAGCGUG | | | AAAGGCAUCU | | | UUUUAGCGUG | |
| | UAC | | | UAC | | | UAC | |
| 525 | AGAUGCCUU | 2101 | 2779-2799 | UGUACACGCU | 528 | 2777-2799 | UAAGAUGCCU | 4007 |
| | UUAGCGUGU | | | AAAAGGCAUC | | | UUUAGCGUGU | |
| | ACA | | | UUA | | | ACA | |
| 526 | AUGCCUUUU | 2102 | 2781-2801 | ACUGUACACG | 529 | 2779-2801 | AGAUGCCUUU | 4008 |
| | AGCGUGUAC | | | CUAAAAGGCA | | | UAGCGUGUAC | |
| | AGU | | | UCU | | | AGU | |
| 527 | UGCCUUUUA | 2103 | 2782-2802 | UACUGUACAC | 530 | 2780-2802 | GAUGCCUUUU | 4009 |
| | GCGUGUACA | | | GCUAAAAGGC | | | AGCGUGUACA | |
| | GUA | | | AUC | | | GUA | |
| 528 | GCCUUUUAG | 2104 | 2783-2803 | GUACUGUACA | 531 | 2781-2803 | AUGCCUUUUA | 4010 |
| | CGUGUACAG | | | CGCUAAAAGG | | | GCGUGUACAG | |
| | UAC | | | CAU | | | UAC | |
| 529 | CCUUUUAGC | 2105 | 2784-2804 | GGUACUGUAC | 532 | 2782-2804 | UGCCUUUUAG | 4011 |
| | GUGUACAGU | | | ACGCUAAAAG | | | CGUGUACAGU | |
| | ACC | | | GCA | | | ACC | |
| 530 | CUUUUAGCG | 2106 | 2785-2805 | GGGUACUGUA | 533 | 2783-2805 | GCCUUUUAGC | 4012 |
| | UGUACAGUA | | | CACGCUAAAA | | | GUGUACAGUA | |
| | CCC | | | GGC | | | CCC | |
| 531 | UUUUAGCGU | 2107 | 2786-2806 | AGGGUACUGU | 534 | 2784-2806 | CCUUUUAGCG | 4013 |
| | GUACAGUAC | | | ACACGCUAAA | | | UGUACAGUAC | |
| | CCU | | | AGG | | | CCU | |
| 532 | UUUAGCGUG | 2108 | 2787-2807 | AAGGGUACUG | 535 | 2785-2807 | CUUUUAGCGU | 4014 |
| | UACAGUACC | | | UACACGCUAA | | | GUACAGUACC | |
| | CUU | | | AAG | | | CUG | |
| 533 | UUAGCGUGU | 2109 | 2788-2808 | ACAGGGUACU | 536 | 2786-2808 | UUUUAGCGUG | 4015 |
| | ACAGUACCC | | | GUACACGCUA | | | UACAGUACCC | |
| | UGU | | | AAA | | | UGG | |
| 534 | UAGCGUGUA | 2110 | 2789-2809 | UCCAGGGUAC | 537 | 2787-2809 | UUUAGCGUGU | 4016 |
| | CAGUACCCU | | | UGUACACGCU | | | ACAGUACCCU | |
| | GGA | | | AAA | | | GGA | |
| 535 | AGCGUGUAC | 2111 | 2790-2810 | AUCCAGGGUA | 538 | 2788-2810 | UUAGCGUGUA | 4017 |
| | AGUACCCUG | | | CUGUACACGC | | | CAGUACCCUG | |
| | GAU | | | UAA | | | GAG | |
| 536 | GUGUACAGU | 2112 | 2792-2812 | UUUCUCCAGG | 539 | 2790-2812 | UUAGCGUGUA | 4018 |
| | ACCCUGGAG | | | GUACUGUACA | | | CAGUACCCUG | |
| | AAA | | | CGC | | | GAG | |
| 537 | UGUACAGUA | 2113 | 2793-2813 | GUUUCUCCAG | 540 | 2791-2813 | CGUGUACAGU | 4019 |
| | CCCUGGAGA | | | GGUACUGUAC | | | ACCCUGGAGA | |
| | AAC | | | ACG | | | AAC | |
| 538 | AUGGCACAG | 2114 | 2824-2844 | AACACUCGAU | 541 | 2822-2844 | AAAUGGCACA | 4020 |
| | UGAUCGAGU | | | CACUGUGCCA | | | GUGAUCGAGU | |
| | GUU | | | UUU | | | GUU | |
| 539 | UGGCACAGU | 2115 | 2825-2845 | AAACACUCGA | 542 | 2823-2845 | AAUGGCACAG | 4021 |
| | GAUCGAGUG | | | UCACUGUGCC | | | UGAUCGAGUG | |
| | UUU | | | AUU | | | UUG | |
| 540 | CACAGUGAU | 2116 | 2828-2848 | AUUCAACACU | 543 | 2826-2848 | GGCACAGUGA | 4022 |
| | CGAGUGUUG | | | CGAUCACUGU | | | UCGAGUGUUG | |
| | AAU | | | GCC | | | AAU | |
| 541 | ACAGUGAUC | 2117 | 2829-2849 | UAUUCAACAC | 544 | 2827-2849 | GCACAGUGAU | 4023 |
| | GAGUGUUGA | | | UCGAUCACUG | | | CGAGUGUUGA | |
| | AUA | | | UGC | | | AUA | |
| 542 | CAGUGAUCG | 2118 | 2830-2850 | UUAUUCAACA | 545 | 2828-2850 | CACAGUGAUC | 4024 |
| | AGUGUUGAA | | | CUCGAUCACU | | | GAGUGUUGAA | |
| | UAA | | | GUG | | | UAA | |
| 543 | AGUGAUCGA | 2119 | 2831-2851 | AUUAUUCAAC | 546 | 2829-2851 | ACAGUGAUCG | 4025 |
| | GUGUUGAAU | | | ACUCGAUCAC | | | AGUGUUGAAU | |
| | AAU | | | UGU | | | AAU | |
| 544 | GUGAUCGAG | 2120 | 2832-2852 | AAUUAUUCAA | 547 | 2830-2852 | CAGUGAUCGA | 4026 |
| | UGUUGAAUA | | | CACUCGAUCA | | | GUGUUGAAUA | |
| | AUU | | | CUG | | | AUG | |
| 545 | GCAGGGAAC | 2121 | 1351-1371 | UGGGCUAGAC | 548 | 1349-1371 | UGGCAGGGAA | 4027 |
| | ACGUCUAGC | | | GUGUUCCCUG | | | CACGUCUAGC | |
| | CCA | | | CCA | | | CCA | |
| 546 | UUCAGAGCA | 2122 | 1547-1567 | GCACCACCAG | 549 | 1545-1567 | AUUUCAGAGC | 4028 |
| | ACCUGGUGG | | | GUUGCUCUGA | | | AACCUGGUGG | |
| | UGC | | | AAU | | | UGC | |
| 547 | AUGACGGUC | 2123 | 1684-1704 | GUCGUCGGAC | 550 | 1682-1704 | CCAUGACGGU | 4029 |
| | AAGUCCGAC | | | UUGACCGUCA | | | CAAGUCCGAC | |
| | GAC | | | UGG | | | GAC | |
| 548 | UGACGGUCA | 2124 | 1685-1705 | AGUCGUCGGA | 551 | 1683-1705 | CAUGACGGUC | 4030 |
| | AGUCCGACG | | | CUUGACCGUC | | | AAGUCCGACG | |
| | ACU | | | AUG | | | ACG | |
| 549 | ACGGUCAAG | 2125 | 1687-1707 | ACCGUCGUCG | 552 | 1685-1707 | UGACGGUCAA | 4031 |
| | UCCGACGAC | | | GACUUGACCG | | | GUCCGACGAC | |
| | GGU | | | UCA | | | GGU | |
| 550 | CAAGUCCGA | 2126 | 1692-1712 | GAGAGACCGU | 553 | 1690-1712 | GUCAAGUCCG | 4032 |
| | CGACGGUCU | | | CGUCGGACUU | | | ACGACGGUCU | |
| | CUC | | | GAC | | | CUC | |
| 551 | CGCUCAAGU | 2127 | 1769-1789 | UGGCCACCAC | 554 | 1767-1789 | CGCGCUCAAG | 4033 |
| | CCGUGGUGG | | | GGACUUGAGC | | | UCCGUGGUGG | |
| | CCA | | | GCG | | | CCA | |
| 552 | UCAAGUCCG | 2128 | 1772-1792 | ACUUGGCCAC | 555 | 1770-1792 | GCUCAAGUCC | 4034 |
| | UGGUGGCCA | | | CACGGACUUG | | | GUGGUGGCCA | |
| | AGU | | | AGC | | | AGU | |
| 553 | UGGUGGCCA | 2129 | 1781-1801 | AGCUCUUGAA | 556 | 1779-1801 | CGUGGUGGCC | 4035 |
| | AGUUCAAGA | | | CUUGGCCACC | | | AAGUUCAAGA | |
| | GCU | | | ACG | | | GCG | |
| 554 | GGUGGCCAA | 2130 | 1782-1802 | UCGCUCUUGA | 557 | 1780-1802 | GUGGUGGCCA | 4036 |
| | GUUCAAGAG | | | ACUUGGCCAC | | | AGUUCAAGAG | |
| | CGA | | | CAC | | | CGA | |
| 555 | AGGAGGACG | 2131 | 1847-1867 | ACUCUUCCUC | 558 | 1845-1867 | AGAGGAGGAC | 4037 |
| | ACGAGGAAG | | | GUCGUCCUCC | | | GACGAGGAAG | |
| | AGU | | | UCU | | | AGG | |
| 556 | GAGGACGAC | 2132 | 1849-1869 | UUCCUCUUCC | 559 | 1847-1869 | AGGAGGACGA | 4038 |
| | GAGGAAGAG | | | UCGUCGUCCU | | | CGAGGAAGAG | |
| | GAA | | | CCU | | | GAA | |
| 557 | CAUCAAGCU | 2133 | 2289-2309 | AACUCCUUCU | 560 | 2287-2309 | CGCAUCAAGC | 4039 |
| | CGAGAAGGA | | | CGAGCUUGAU | | | UCGAGAAGGA | |
| | GUU | | | GCG | | | GUU | |
| 558 | AUCAAGCUC | 2134 | 2290-2310 | GAACUCCUUC | 561 | 2288-2310 | GCAUCAAGCU | 4040 |
| | GAGAAGGAG | | | UCGAGCUUGA | | | CGAGAAGGAG | |
| | UUC | | | UGC | | | UUC | |
| 559 | CAAGCUCGA | 2135 | 2292-2312 | UCGAACUCCU | 562 | 2290-2312 | AUCAAGCUCG | 4041 |
| | GAAGGAGUU | | | UCUCGAGCUU | | | AGAAGGAGUU | |
| | CGA | | | GAU | | | CGA | |
| 560 | UCCUUAGCU | 2136 | 2402-2422 | UGGAGUCUCC | 563 | 2400-2422 | CUUCCUUAGC | 4042 |
| | UCGGAGACU | | | GAAGCUAAGG | | | UUCGGAGACU | |
| | CCA | | | AAG | | | CCA | |
| 561 | CCUUAGCUU | 2137 | 2403-2423 | AUGGAGUCUC | 564 | 2401-2423 | UUCCUUAGCU | 4043 |
| | CGGAGACUC | | | CGAAGCUAAG | | | UCGGAGACUC | |
| | CAU | | | GAA | | | CAG | |
| 562 | UAGCUUCGG | 2138 | 2406-2426 | UGUCUGGAGU | 565 | 2404-2426 | CUUAGCUUCG | 4044 |
| | AGACUCCAG | | | CUCCGAAGCU | | | GAGACUCCAG | |
| | ACA | | | AAG | | | ACA | |
| 563 | AGCUUCGGA | 2139 | 2407-2427 | UUGUCUGGAG | 566 | 2405-2427 | UUAGCUUCGG | 4045 |
| | GACUCCAGA | | | UCUCCGAAGC | | | AGACUCCAGA | |
| | CAA | | | UAA | | | CAA | |
| 564 | GCUUCGGAG | 2140 | 2408-2428 | AUUGUCUGGA | 567 | 2406-2428 | UAGCUUCGGA | 4046 |
| | ACUCCAGAC | | | GUCUCCGAAG | | | GACUCCAGAC | |
| | AAU | | | CUA | | | AAU | |
| 565 | CUUCGGAGA | 2141 | 2409-2429 | GAUUGUCUGG | 568 | 2407-2429 | AGCUUCGGAG | 4047 |
| | CUCCAGACA | | | AGUCUCCGAA | | | ACUCCAGACA | |
| | AUC | | | GCU | | | AUC | |
| 566 | UUGCCUCCU | 2142 | 2410-2430 | AGUGCUCCGA | 569 | 2408-2430 | UUUUGCCUCC | 4048 |
| | CGUCGGAGC | | | CGAGGAGGCA | | | UCGUCGGAGC | |
| | ACU | | | AAA | | | ACU | |
| 567 | UGCCUCCUC | 2143 | 2411-2431 | GAGUGCUCCG | 570 | 2409-2431 | UUUGCCUCCU | 4049 |
| | GUCGGAGCA | | | ACGAGGAGGC | | | CGUCGGAGCA | |
| | CUC | | | AAA | | | CUC | |
| 568 | GGACAUUCU | 2144 | 558-578 | UCGAUAAAAA | 571 | 556-578 | GGGGACAUUC | 4050 |
| | UAUUUUUAU | | | UAAGAAUGUC | | | UUAUUUUUAU | |
| | CGA | | | CCC | | | CGA | |
| 569 | GACAUUCUU | 2145 | 559-579 | CUCGAUAAAA | 572 | 557-579 | GGGACAUUCU | 4051 |
| | AUUUUUAUC | | | AUAAGAAUGU | | | UAUUUUUAUC | |
| | GAG | | | CCC | | | GAG | |
| 570 | ACAUUCUUA | 2146 | 560-580 | GCUCGAUAAA | 573 | 558-580 | GGACAUUCUU | 3550 |
| | UUUUUAUCG | | | AAUAAGAAUG | | | AUUUUUAUCG | |
| | AGC | | | UCC | | | AGC | |
| 571 | GCUCGAUAA | 2147 | 561-581 | UGCUCGAUAA | 574 | 558-581 | GACAUUCUUA | 4052 |
| | AAAUAAGAA | | | AAAUAAGAAU | | | UUUUUAUCGA | |
| | UGU | | | GUC | | | GCA | |
| 572 | AUUCUUAUU | 2148 | 562-582 | GUGCUCGAUA | 575 | 560-582 | ACAUUCUUAU | 3558 |
| | UUUAUCGAG | | | AAAAUAAGAA | | | UUUUAUCGAG | |
| | CAC | | | UGU | | | CAC | |
| 573 | UUCUUAUUU | 2149 | 563-583 | UGUGCUCGAU | 576 | 561-583 | CAUUCUUAUU | 4053 |
| | UUAUCGAGC | | | AAAAAUAAGA | | | UUUAUCGAGC | |
| | ACA | | | AUG | | | ACA | |
| 574 | UCUUAUUUU | 2150 | 564-584 | UUGUGCUCGA | 577 | 562-584 | AUUCUUAUUU | 4054 |
| | UAUCGAGCA | | | UAAAAAUAAG | | | UUAUCGAGCA | |
| | CAA | | | AAU | | | CAA | |
| 575 | CUUAUUUUU | 2151 | 565-585 | UUUGUGCUCG | 578 | 563-585 | UUCUUAUUUU | 4055 |
| | AUCGAGCAC | | | AUAAAAAUAA | | | UAUCGAGCAC | |
| | AAA | | | GAA | | | AAA | |
| 576 | UUAUUUUUA | 2152 | 566-586 | GUUUGUGCUC | 579 | 564-586 | UCUUAUUUUU | 4056 |
| | UCGAGCACA | | | GAUAAAAAUA | | | AUCGAGCACA | |
| | AAC | | | AGA | | | AAC | |
| 577 | UAUUUUUAU | 2153 | 567-587 | CGUUUGUGCU | 580 | 565-587 | CUUAUUUUUA | 4057 |
| | CGAGCACAA | | | CGAUAAAAAU | | | UCGAGCACAA | |
| | ACG | | | AAG | | | ACG | |
| 578 | AUUUUUAUC | 2154 | 568-588 | CCGUUUGUGC | 581 | 566-588 | UUAUUUUUAU | 4058 |
| | GAGCACAAA | | | UCGAUAAAAA | | | CGAGCACAAA | |
| | CGG | | | UAA | | | CGG | |
| 579 | UUUUUAUCG | 2155 | 569-589 | UCCGUUUGUG | 582 | 567-589 | UAUUUUUAUC | 4059 |
| | AGCACAAAC | | | CUCGAUAAAA | | | GAGCACAAAC | |
| | GGA | | | AUA | | | GGA | |
| 580 | UUUUAUCGA | 2156 | 570-590 | UUCCGUUUGU | 583 | 568-590 | AUUUUUAUCG | 4060 |
| | GCACAAACG | | | GCUCGAUAAA | | | AGCACAAACG | |
| | GAA | | | AAU | | | GAA | |
| 581 | UUUAUCGAG | 2157 | 571-591 | UUUCCGUUUG | 584 | 569-591 | UUUUUAUCGA | 4061 |
| | CACAAACGG | | | UGCUCGAUAA | | | GCACAAACGG | |
| | AAA | | | AAA | | | AAA | |
| 582 | UUAUCGAGC | 2158 | 572-592 | GUUUCCGUUU | 585 | 570-592 | UUUUAUCGAG | 4062 |
| | ACAAACGGA | | | GUGCUCGAUA | | | CACAAACGGA | |
| | AAC | | | AAA | | | AAC | |
| 583 | UAUGCCCCG | 2159 | 853-873 | ACAAAUACCC | 586 | 851-873 | AAUAUGCCCC | 4063 |
| | CAGGGUAUU | | | UGCGGGGCAU | | | GCAGGGUAUU | |
| | UGU | | | AUU | | | UGU | |
| 584 | CGCAGGGUA | 2160 | 860-880 | CAUCUUUACA | 587 | 858-880 | CCCGCAGGGU | 4064 |
| | UUUGUAAAG | | | AAUACCCUGC | | | AUUUGUAAAG | |
| | AUG | | | GGG | | | AUG | |
| 585 | GCAGGGUAU | 2161 | 861-881 | UCAUCUUUAC | 588 | 859-881 | CCGCAGGGUA | 4065 |
| | UUGUAAAGA | | | AAAUACCCUG | | | UUUGUAAAGA | |
| | UGA | | | CGG | | | UGA | |
| 586 | CAGGGUAUU | 2162 | 862-882 | CUCAUCUUUA | 589 | 860-882 | CGCAGGGUAU | 4066 |
| | UGUAAAGAU | | | CAAAUACCCU | | | UUGUAAAGAU | |
| | GAG | | | GCG | | | GAG | |
| 587 | AGGGUAUUU | 2163 | 863-883 | GCUCAUCUUU | 590 | 861-883 | GCAGGGUAUU | 4067 |
| | GUAAAGAUG | | | ACAAAUACCC | | | UGUAAAGAUG | |
| | AGC | | | UGC | | | AGC | |
| 588 | GGUAUUUGU | 2164 | 865-885 | GGGCUCAUCU | 591 | 863-885 | AGGGUAUUUG | 4068 |
| | AAAGAUGAG | | | UUACAAAUAC | | | UAAAGAUGAG | |
| | CCC | | | CUG | | | CCC | |
| 589 | GUAUUUGUA | 2165 | 866-886 | UGGGCUCAUC | 592 | 864-886 | GGGUAUUUGU | 4069 |
| | AAGAUGAGC | | | UUUACAAAUA | | | AAAGAUGAGC | |
| | CCA | | | CCU | | | CCA | |
| 590 | UAUUUGUAA | 2166 | 867-887 | CUGGGCUCAU | 593 | 865-887 | GGUAUUUGUA | 4070 |
| | AGAUGAGCC | | | CUUUACAAAU | | | AAGAUGAGCC | |
| | CAG | | | ACC | | | CAG | |
| 591 | AUUUGUAAA | 2167 | 868-888 | GCUGGGCUCA | 594 | 866-888 | GUAUUUGUAA | 4071 |
| | GAUGAGCCC | | | UCUUUACAAA | | | AGAUGAGCCC | |
| | AGC | | | UAC | | | AGC | |
| 592 | UUUGUAAAG | 2168 | 869-889 | UGCUGGGCUC | 595 | 867-889 | UAUUUGUAAA | 4072 |
| | AUGAGCCCA | | | AUCUUUACAA | | | GAUGAGCCCA | |
| | GCA | | | AUA | | | GCA | |
| 593 | UUGUAAAGA | 2169 | 870-890 | CUGCUGGGCU | 596 | 868-890 | AUUUGUAAAG | 4073 |
| | UGAGCCCAG | | | CAUCUUUACA | | | AUGAGCCCAG | |
| | CAG | | | AAU | | | CAG | |
| 594 | UGUAAAGAU | 2170 | 871-891 | GCUGCUGGGC | 597 | 869-891 | UUUGUAAAGA | 4074 |
| | GAGCCCAGC | | | UCAUCUUUAC | | | UGAGCCCAGC | |
| | AGC | | | AAA | | | AGC | |
| 595 | GUAAAGAUG | 2171 | 872-892 | AGCUGCUGGG | 598 | 870-892 | UUGUAAAGAU | 4075 |
| | AGCCCAGCA | | | CUCAUCUUUA | | | GAGCCCAGCA | |
| | GCU | | | CAA | | | GCU | |
| 596 | UAAAGAUGA | 2172 | 873-893 | UAGCUGCUGG | 599 | 871-893 | UGUAAAGAUG | 4076 |
| | GCCCAGCAG | | | GCUCAUCUUU | | | AGCCCAGCAG | |
| | CUA | | | ACA | | | CUA | |
| 597 | AAGAUGAGC | 2173 | 875-895 | UGUAGCUGCU | 600 | 872-895 | UAAAGAUGAG | 4077 |
| | CCAGCAGCU | | | GGGCUCAUCU | | | CCCAGCAGCU | |
| | ACA | | | UUA | | | ACA | |
| 598 | AGAUGAGCC | 2174 | 876-896 | GUGUAGCUGC | 601 | 874-896 | AAAGAUGAGC | 4078 |
| | CAGCAGCUA | | | UGGGCUCAUC | | | CCAGCAGCUA | |
| | CAC | | | UUU | | | CAC | |
| 599 | GAUGAGCCC | 2175 | 877-897 | UGUGUAGCUG | 602 | 875-897 | AAGAUGAGCC | 4079 |
| | AGCAGCUAC | | | CUGGGCUCAU | | | CAGCAGCUAC | |
| | ACA | | | CUU | | | ACA | |
| 600 | AUGAGCCCA | 2176 | 878-898 | AUGUGUAGCU | 603 | 876-898 | AGAUGAGCCC | 4080 |
| | GCAGCUACA | | | GCUGGGCUCA | | | AGCAGCUACA | |
| | CAU | | | UCU | | | CAU | |
| 601 | UGAGCCCAG | 2177 | 879-899 | CAUGUGUAGC | 604 | 877-899 | GAUGAGCCCA | 4081 |
| | CAGCUACAC | | | UGCUGGGCUC | | | GCAGCUACAC | |
| | AUG | | | AUC | | | AUG | |
| 602 | ACACAUGUA | 2178 | 893-913 | GUUUGCAAGU | 605 | 891-913 | CUACACAUGU | 4082 |
| | CAACUUGCA | | | UGUACAUGUG | | | ACAACUUGCA | |
| | AAC | | | UAG | | | AAC | |
| 603 | ACAUGUACA | 2179 | 895-915 | CUGUUUGCAA | 606 | 893-915 | ACACAUGUAC | 4083 |
| | ACUUGCAAA | | | GUUGUACAUG | | | AACUUGCAAA | |
| | CAG | | | UGU | | | CAG | |
| 604 | UGCAACACG | 2180 | 941-961 | GAGUGUUCUG | 607 | 939-961 | CUUGCAACAC | 4084 |
| | CACAGAACA | | | UGCGUGUUGC | | | GCACAGAACA | |
| | CUC | | | AAG | | | CUC | |
| 605 | GUACAGUAC | 2181 | 2817-2837 | UGUUUCUCCA | 608 | 2815-2837 | GUGUACAGUA | 4085 |
| | CCUGGAGAA | | | GGGUACUGUA | | | CCCUGGAGAA | |
| | ACA | | | CAC | | | ACA | |
| 606 | UACAGUACC | 2182 | 2818-2838 | GUGUUUCUCC | 609 | 2816-2838 | UGUACAGUAC | 4086 |
| | CUGGAGAAA | | | AGGGUACUGU | | | CCUGGAGAAA | |
| | CAC | | | ACA | | | CAC | |
| 607 | ACAGUACCC | 2183 | 2819-2839 | UGUGUUUCUC | 610 | 2817-2839 | GUACAGUACC | 4087 |
| | UGGAGAAAC | | | CAGGGUACUG | | | CUGGAGAAAC | |
| | ACA | | | UAC | | | ACA | |
| 608 | CAGUACCCU | 2184 | 2820-2840 | AUGUGUUUCU | 611 | 2818-2840 | UACAGUACCC | 4088 |
| | GGAGAAACA | | | CCAGGGUACU | | | UGGAGAAACA | |
| | CAU | | | GUA | | | CAU | |
| 609 | AGUACCCUG | 2185 | 2821-2841 | CAUGUGUUUC | 612 | 2819-2841 | ACAGUACCCU | 4089 |
| | GAGAAACAC | | | UCCAGGGUAC | | | GGAGAAACAC | |
| | AUG | | | UGU | | | AUG | |
| 610 | GUACCCUGG | 2186 | 2822-2842 | UCAUGUGUUU | 613 | 2820-2842 | CAGUACCCUG | 4090 |
| | AGAAACACA | | | CUCCAGGGUA | | | GAGAAACACA | |
| | UGA | | | CUG | | | UGA | |
| 611 | UACCCUGGA | 2187 | 2823-2843 | UUCAUGUGUU | 614 | 2821-2843 | AGUACCCUGG | 4091 |
| | GAAACACAU | | | UCUCCAGGGU | | | AGAAACACAU | |
| | GAA | | | ACU | | | GAA | |
| 612 | ACCCUGGAG | 2188 | 2824-2844 | UUUCAUGUGU | 615 | 2822-2844 | GUACCCUGGA | 4092 |
| | AAACACAUG | | | UUCUCCAGGG | | | GAAACACAUG | |
| | AAA | | | UAC | | | AAA | |
| 613 | CCCUGGAGA | 2189 | 2825-2845 | UUUUCAUGUG | 616 | 2823-2845 | UACCCUGGAG | 4093 |
| | AACACAUGA | | | UUUCUCCAGG | | | AAACACAUGA | |
| | AAA | | | GUA | | | AAA | |
| 614 | CCUGGAGAA | 2190 | 2826-2846 | UUUUUCAUGU | 617 | 2824-2846 | ACCCUGGAGA | 4094 |
| | ACACAUGAA | | | GUUUCUCCAG | | | AACACAUGAA | |
| | AAA | | | GGU | | | AAA | |
| 615 | CUGGAGAAA | 2191 | 2827-2847 | UUUUUUCAUG | 618 | 2825-2847 | CCCUGGAGAA | 4095 |
| | CACAUGAAA | | | UGUUUCUCCA | | | ACACAUGAAA | |
| | AAA | | | GGG | | | AAA | |
| 616 | UGGAGAAAC | 2192 | 2828-2848 | AUUUUUUCAU | 619 | 2826-2848 | CCUGGAGAAA | 4096 |
| | ACAUGAAAA | | | GUGUUUCUCC | | | CACAUGAAAA | |
| | AAU | | | AGG | | | AAU | |
| 617 | GGAGAAACA | 2193 | 2829-2849 | CAUUUUUUCA | 620 | 2827-2849 | CUGGAGAAAC | 4097 |
| | CAUGAAAAA | | | UGUGUUUCUC | | | ACAUGAAAAA | |
| | AUG | | | CAG | | | AUG | |
| 618 | AACCAGACC | 2194 | 473-493 | UCAACGGGCC | 621 | 471-493 | UGAACCAGAC | 4098 |
| | ACGGCCCGU | | | GUGGUCUGGU | | | CACGGCCCGU | |
| | UGA | | | UCA | | | UGG | |
| 619 | ACCAGACCA | 2195 | 474-494 | UCCAACGGGC | 622 | 472-494 | GAACCAGACC | 4099 |
| | CGGCCCGUU | | | CGUGGUCUGG | | | ACGGCCCGUU | |
| | GGA | | | UUC | | | GGG | |
| 620 | GACAUUCUU | 2196 | 559-479 | UUCGAUAAAA | 623 | 557-479 | GGGACAUUCU | 4100 |
| | AUUUUUAUC | | | AUAAGAAUGU | | | UAUUUUUAUC | |
| | GAA | | | CCC | | | GAG | |
| 621 | CAUUCUUAU | 2197 | 561-581 | UGCUCGAUAA | 574 | 559-581 | GACAUUCUUA | 4101 |
| | UUUUAUCGA | | | AAAUAAGAAU | | | UUUUUAUCGA | |
| | GCA | | | GUC | | | GCA | |
| 622 | UUAUUUUUA | 2198 | 566-586 | UUUUGUGCUC | 624 | 564-586 | UCUUAUUUUU | 4102 |
| | UCGAGCACA | | | GAUAAAAAUA | | | AUCGAGCACA | |
| | AAA | | | AGA | | | AA | |
| 623 | UAUUUUUAU | 2199 | 567-587 | UGUUUGUGCU | 625 | 565-587 | CUUAUUUUUA | 4103 |
| | CGAGCACAA | | | CGAUAAAAAU | | | UCGAGCACAA | |
| | ACA | | | AAG | | | ACG | |
| 624 | AUUUUUAUC | 2200 | 568-588 | UCGUUUGUGC | 626 | 566-588 | UUAUUUUUAU | 4104 |
| | GAGCACAAA | | | UCGAUAAAAA | | | CGAGCACAAA | |
| | CGA | | | UAA | | | CGG | |
| 625 | UUAUCGAGC | 2201 | 572-592 | UUUUCCGUUU | 627 | 569-592 | UUUUAUCGAG | 4105 |
| | ACAAACGGA | | | GUGCUCGAUA | | | CACAAACGGA | |
| | AAA | | | AAA | | | AAC | |
| 626 | CCUUCCCCU | 2202 | 637-657 | UUCGAUUGGU | 628 | 635-657 | CACCUUCCCC | 4106 |
| | UCACCAAUC | | | GAAGGGGAAG | | | UUCACCAAUC | |
| | GAA | | | GUG | | | GA | |
| 627 | CUUCACCAA | 2203 | 644-664 | UUUUCAUCUC | 629 | 642-664 | CCCUUCACCA | 4107 |
| | UCGAGAUGA | | | GAUUGGUGAA | | | AUCGAGAUGA | |
| | AAA | | | GGG | | | AAA | |
| 628 | AAAGCAUCC | 2204 | 664-684 | UUCCACGGGA | 630 | 662-684 | AAAAAGCAUC | 4108 |
| | AAUCCCGUG | | | UUGGAUGCUU | | | CAAUCCCGUG | |
| | GAA | | | UUU | | | GAG | |
| 629 | GCAUCCAAU | 2205 | 667-687 | UACCUCCACG | 631 | 665-687 | AAGCAUCCAA | 4109 |
| | CCCGUGGAG | | | GGAUUGGAUG | | | UCCCGUGGAG | |
| | GUA | | | CUU | | | GUU | |
| 630 | UCACGCCAG | 2206 | 698-718 | UAUCGUCAUC | 632 | 696-718 | GGUCACGCCA | 4110 |
| | AGGAUGACG | | | CUCUGGCGUG | | | GAGGAUGACG | |
| | AUA | | | ACC | | | AUU | |
| 631 | AUGACGAUU | 2207 | 710-730 | UCGUUGAUAA | 633 | 708-730 | GGAUGACGAU | 4111 |
| | GUUUAUCAA | | | ACAAUCGUCA | | | UGUUUAUCAA | |
| | CGA | | | UCC | | | CGU | |
| 632 | UGACGAUUG | 2208 | 711-731 | UACGUUGAUA | 634 | 709-731 | GAUGACGAUU | 4112 |
| | UUUAUCAAC | | | AACAAUCGUC | | | GUUUAUCAAC | |
| | GUA | | | AUC | | | GUC | |
| 633 | UUGUUUAUC | 2209 | 717-737 | UUAGAUGACG | 635 | 715-737 | GAUUGUUUAU | 4113 |
| | AACGUCAUC | | | UUGAUAAACA | | | CAACGUCAUC | |
| | UAA | | | AUC | | | UAG | |
| 634 | UAUGCCCCG | 2210 | 853-873 | UCAAAUACCC | 636 | 851-873 | AAUAUGCCCC | 4114 |
| | CAGGGUAUU | | | UGCGGGGCAU | | | GCAGGGUAUU | |
| | UGA | | | AUU | | | UGU | |
| 635 | CGCAGGGUA | 2211 | 860-880 | UAUCUUUACA | 637 | 858-880 | CCCGCAGGGU | 4115 |
| | UUUGUAAAG | | | AAUACCCUGC | | | AUUUGUAAAG | |
| | AUA | | | GGG | | | AUG | |
| 636 | CAGGGUAUU | 2212 | 862-882 | UUCAUCUUUA | 638 | 860-882 | CGCAGGGUAU | 4116 |
| | UGUAAAGAU | | | CAAAUACCCU | | | UUGUAAAGAU | |
| | GAA | | | GCG | | | GAG | |
| 637 | AGGGUAUUU | 2213 | 863-883 | UCUCAUCUUU | 639 | 861-883 | GCAGGGUAUU | 4117 |
| | GUAAAGAUG | | | ACAAAUACCC | | | UGUAAAGAUG | |
| | AGA | | | UGC | | | AGC | |
| 638 | GGUAUUUGU | 2214 | 865-885 | UGGCUCAUCU | 640 | 863-885 | AGGGUAUUUG | 4118 |
| | AAAGAUGAG | | | UUACAAAUAC | | | UAAAGAUGAG | |
| | CCA | | | CUG | | | CCC | |
| 639 | UAUUUGUAA | 2215 | 867-887 | UUGGGCUCAU | 641 | 865-887 | GGUAUUUGUA | 4119 |
| | AGAUGAGCC | | | CUUUACAAAU | | | AAGAUGAGCC | |
| | CAA | | | ACC | | | CAG | |
| 640 | AUUUGUAAA | 2216 | 868-888 | UCUGGGCUCA | 642 | 866-888 | GUAUUUGUAA | 4120 |
| | GAUGAGCCC | | | UCUUUACAAA | | | AGAUGAGCCC | |
| | AGA | | | UAC | | | AGC | |
| 641 | UUGUAAAGA | 2217 | 870-890 | UUGCUGGGCU | 643 | 868-890 | AUUUGUAAAG | 4121 |
| | UGAGCCCAG | | | CAUCUUUACA | | | AUGAGCCCAG | |
| | CAA | | | AAU | | | CAG | |
| 642 | UGUAAAGAU | 2218 | 871-891 | UCUGCUGGGC | 644 | 869-891 | UUUGUAAAGA | 4122 |
| | GAGCCCAGC | | | UCAUCUUUAC | | | UGAGCCCAGC | |
| | AGA | | | AAA | | | AGC | |
| 643 | GUAAAGAUG | 2219 | 872-892 | UGCUGCUGGG | 645 | 870-892 | UUGUAAAGAU | 4123 |
| | AGCCCAGCA | | | CUCAUCUUUA | | | GAGCCCAGCA | |
| | GCA | | | CAA | | | GCU | |
| 644 | AGAUGAGCC | 2220 | 876-896 | UUGUAGCUGC | 646 | 874-896 | AAAGAUGAGC | 4124 |
| | CAGCAGCUA | | | UGGGCUCAUC | | | CCAGCAGCUA | |
| | CAA | | | UUU | | | CAC | |
| 645 | AUGAGCCCA | 2221 | 878-898 | UUGUGUAGCU | 647 | 876-898 | AGAUGAGCCC | 4125 |
| | GCAGCUACA | | | GCUGGGCUCA | | | AGCAGCUACA | |
| | CAA | | | UCU | | | CAU | |
| 646 | UGAGCCCAG | 2222 | 879-898 | UAUGUGUAGC | 648 | 877-898 | GAUGAGCCCA | 4126 |
| | CAGCUACAC | | | UGCUGGGCUC | | | GCAGCUACAC | |
| | AUA | | | AUC | | | AUG | |
| 647 | ACACAUGUA | 2223 | 893-913 | UUUUGCAAGU | 649 | 891-913 | CUACACAUGU | 4127 |
| | CAACUUGCA | | | UGUACAUGUG | | | ACAACUUGCA | |
| | AAA | | | UAG | | | AAC | |
| 648 | ACAUGUACA | 2224 | 895-915 | UUGUUUGCAA | 650 | 893-915 | ACACAUGUAC | 4128 |
| | ACUUGCAAA | | | GUUGUACAUG | | | AACUUGCAAA | |
| | CAA | | | UGU | | | CAG | |
| 649 | UGCAACACG | 2225 | 941-961 | UAGUGUUCUG | 651 | 939-961 | CUUGCAACAC | 4129 |
| | CACAGAACA | | | UGCGUGUUGC | | | GCACAGAACA | |
| | CUA | | | AAG | | | CUC | |
| 650 | CAACACGCA | 2226 | 943-963 | UUGAGUGUUC | 652 | 941-963 | UGCAACACGC | 4130 |
| | CAGAACACU | | | UGUGCGUGUU | | | ACAGAACACU | |
| | CAA | | | GCA | | | CAU | |
| 651 | AACACGCAC | 2227 | 944-964 | UAUGAGUGUU | 653 | 942-964 | GCAACACGCA | 3553 |
| | AGAACACUC | | | CUGUGCGUGU | | | CAGAACACUC | |
| | AUA | | | UGC | | | AUG | |
| 652 | ACACGCACA | 2228 | 945-965 | UCAUGAGUGU | 654 | 943-965 | CAACACGCAC | 4131 |
| | GAACACUCA | | | UCUGUGCGUG | | | AGAACACUCA | |
| | UGA | | | UUG | | | UGG | |
| 653 | CCCUGACCC | 2229 | 1001-1021 | UACCAACCCG | 655 | 999-1021 | UCCCCUGACC | 4132 |
| | CGCGGGUUG | | | CGGGGUCAGG | | | CCGCGGGUUG | |
| | GUA | | | GGA | | | GUA | |
| 654 | CUGACCCCG | 2230 | 1003-1023 | UAUACCAACC | 656 | 1001-1023 | CCCUGACCCC | 4133 |
| | CGGGUUGGU | | | CGCGGGGUCA | | | GCGGGUUGGU | |
| | AUA | | | GGG | | | AUC | |
| 655 | GACCCCGCG | 2231 | 1005-1025 | UGGAUACCAA | 657 | 1003-1025 | CUGACCCCGC | 4134 |
| | GGUUGGUAU | | | CCCGCGGGGU | | | GGGUUGGUAU | |
| | CCA | | | CAG | | | CCC | |
| 656 | ACCCCGCGG | 2232 | 1106-1126 | UGGGAUACCA | 658 | 1104-1126 | UGACCCCGCG | 4135 |
| | GUUGGUAUC | | | ACCCGCGGGG | | | GGUUGGUAUC | |
| | CCA | | | UCA | | | CCU | |
| 657 | GUAUCCCUU | 2233 | 1019-1039 | UACCUAGUCC | 659 | 1017-1039 | UGGUAUCCCU | 4136 |
| | CAGGACUAG | | | UGAAGGGAUA | | | UCAGGACUAG | |
| | GUA | | | CCA | | | GUG | |
| 658 | CAGACAAUA | 2234 | 1082-1102 | UGUUAAAGGG | 660 | 1080-1102 | UGCAGACAAU | 4137 |
| | ACCCCUUUA | | | GUUAUUGUCU | | | AACCCCUUUA | |
| | ACA | | | GCA | | | ACC | |
| 659 | AGACAAUAA | 2235 | 1083-1103 | UGGUUAAAGG | 661 | 1081-1103 | GCAGACAAUA | 4138 |
| | CCCCUUUAA | | | GGUUAUUGUC | | | ACCCCUUUAA | |
| | CCA | | | UGC | | | CCU | |
| 660 | AAUACCAGG | 2236 | 1110-1130 | UUCGAUACUG | 662 | 1108-1130 | AGAAUACCAG | 4139 |
| | AUCAGUAUC | | | AUCCUGGUAU | | | GAUCAGUAUC | |
| | GAA | | | UCU | | | GAG | |
| 661 | CCUGUUUAG | 2237 | 1176-1196 | UUCGGUGGUG | 663 | 1174-1196 | CCCCUGUUUA | 4140 |
| | UCCACCACC | | | GACUAAACAG | | | GUCCACCACC | |
| | GAA | | | GGG | | | GAG | |
| 662 | CAAGUCCAA | 2238 | 1506-1526 | UACUCGCAUG | 664 | 1504-1526 | GUCAAGUCCA | 4141 |
| | GUCAUGCGA | | | ACUUGGACUU | | | AGUCAUGCGA | |
| | GUA | | | GAC | | | GUU | |
| 663 | AAGUCCAAG | 2239 | 1508-1528 | UAACUCGCAU | 665 | 1506-1528 | UCAAGUCCAA | 4142 |
| | UCAUGCGAG | | | GACUUGGACU | | | GUCAUGCGAG | |
| | UUA | | | UGA | | | UUC | |
| 664 | GUGCGACCA | 2240 | 1611-1631 | UGGGUGCACG | 666 | 1609-1631 | CUGUGCGACC | 4143 |
| | CGCGUGCAC | | | CGUGGUCGCA | | | ACGCGUGCAC | |
| | CCA | | | CAG | | | CCA | |
| 665 | CCAUGACGG | 2241 | 1682-1702 | UGUCGGACUU | 667 | 1680-1702 | CCCCAUGACG | 4144 |
| | UCAAGUCCG | | | GACCGUCAUG | | | GUCAAGUCCG | |
| | ACA | | | GGG | | | ACG | |
| 666 | AUGACGGUC | 2242 | 1684-1704 | UUCGUCGGAC | 668 | 1682-1704 | CCAUGACGGU | 4145 |
| | AAGUCCGAC | | | UUGACCGUCA | | | CAAGUCCGAC | |
| | GAA | | | UGG | | | GAC | |
| 667 | UGACGGUCA | 2243 | 2124-2144 | UGUCGUCGGA | 669 | 2122-2144 | CAUGACGGUC | 4146 |
| | AGUCCGACG | | | CUUGACCGUC | | | AAGUCCGACG | |
| | ACA | | | AUG | | | ACG | |
| 668 | GACGGUCAA | 2244 | 1686-1706 | UCGUCGUCGG | 670 | 1684-1706 | AUGACGGUCA | 4147 |
| | GUCCGACGA | | | ACUUGACCGU | | | AGUCCGACGA | |
| | CGA | | | CAU | | | CGG | |
| 669 | ACGGUCAAG | 2245 | 1687-1707 | UCCGUCGUCG | 671 | 1685-1707 | UGACGGUCAA | 4148 |
| | UCCGACGAC | | | GACUUGACCG | | | GUCCGACGAC | |
| | GGA | | | UCA | | | GGU | |
| 670 | CGGUCAAGU | 2246 | 1688-1708 | UACCGUCGUC | 672 | 1686-1708 | GACGGUCAAG | 4149 |
| | CCGACGACG | | | GGACUUGACC | | | UCCGACGACG | |
| | GUA | | | GUC | | | GUC | |
| 671 | GGUCAAGUC | 2247 | 1689-1709 | UGACCGUCGU | 673 | 1687-1709 | ACGGUCAAGU | 4150 |
| | CGACGACGG | | | CGGACUUGAC | | | CCGACGACGG | |
| | UCA | | | CGU | | | UCU | |
| 672 | CAAGUCCGA | 2248 | 1692-1712 | UAGAGACCGU | 674 | 1690-1712 | GUCAAGUCCG | 4151 |
| | CGACGGUCU | | | CGUCGGACUU | | | ACGACGGUCU | |
| | CUA | | | GAC | | | CUC | |
| 673 | AGCAGCGCG | 2249 | 1762-1782 | UACGGACUUG | 675 | 1760-1782 | CCAGCAGCGC | 4152 |
| | CUCAAGUCC | | | AGCGCGCUGC | | | GCUCAAGUCC | |
| | GUA | | | UGG | | | GUG | |
| 674 | GCAGCGCGC | 2250 | 1763-1783 | UCACGGACUU | 676 | 1761-1783 | CAGCAGCGCG | 4153 |
| | UCAAGUCCG | | | GAGCGCGCUG | | | CUCAAGUCCG | |
| | UGA | | | CUG | | | UGG | |
| 675 | GAGAACGAC | 2251 | 1801-1821 | UAUCAGGUUG | 677 | 1799-1821 | GCGAGAACGA | 4154 |
| | CCCAACCUG | | | GGGUCGUUCU | | | CCCCAACCUG | |
| | AUA | | | CGC | | | AUC | |
| 676 | CUGACGGAG | 2252 | 1897-1917 | UACCCUCUCG | 678 | 1897-1917 | AGCUGACGGA | 4155 |
| | AGCGAGAGG | | | CUCUCCGUCA | | | GAGCGAGAGG | |
| | GUA | | | GCU | | | GUG | |
| 677 | AGAGCGAGA | 2253 | 1904-1924 | UGUAGUCCAC | 679 | 1904-1924 | GGAGAGCGAG | 4156 |
| | GGGUGGACU | | | CCUCUCGCUC | | | AGGGUGGACU | |
| | ACA | | | UCC | | | ACG | |
| 678 | GCGAGAGGG | 2254 | 1907-1927 | UGCCGUAGUC | 680 | 1907-1927 | GAGCGAGAGG | 4157 |
| | UGGACUACG | | | CACCCUCUCG | | | GUGGACUACG | |
| | GCA | | | CUC | | | GCU | |
| 679 | CGAGAGGGU | 2255 | 1908-1928 | UAGCCGUAGU | 681 | 1908-1928 | AGCGAGAGGG | 4158 |
| | GGACUACGG | | | CCACCCUCUC | | | UGGACUACGG | |
| | CUA | | | GCU | | | CUU | |
| 680 | GAGGGUGGA | 2256 | 1911-1931 | UCGAAGCCGU | 682 | 1911-1931 | GAGAGGGUGG | 4159 |
| | CUACGGCUU | | | AGUCCACCCU | | | ACUACGGCUU | |
| | CGA | | | CUC | | | CGG | |
| 681 | CACGAGAAC | 2257 | 1957-1977 | UCCCCGCGAG | 683 | 1957-1977 | ACCACGAGAA | 4160 |
| | AGCUCGCGG | | | CUGUUCUCGU | | | CAGCUCGCGG | |
| | GGA | | | GGU | | | GGC | |
| 682 | GGGCGAGAA | 2258 | 2082-2102 | UCGCGCUUAU | 684 | 2082-2102 | CUGGGCGAGA | 4161 |
| | GCAUAAGCG | | | GCUUCUCGCC | | | AGCAUAAGCG | |
| | CGA | | | CAG | | | CGG | |
| 683 | GAGAAGCAU | 2259 | 2086-2106 | UUGGCCGCGC | 685 | 2086-2106 | GCGAGAAGCA | 4162 |
| | AAGCGCGGC | | | UUAUGCUUCU | | | UAAGCGCGGC | |
| | CAA | | | CGC | | | CAC | |
| 684 | UGCGACGAA | 2260 | 2137-2157 | UGCCACCGAG | 686 | 2137-2157 | CUUGCGACGA | 4163 |
| | GACUCGGUG | | | UCUUCGUCGC | | | AGACUCGGUG | |
| | GCA | | | AAG | | | GCC | |
| 685 | GACUCGGUG | 2261 | 2146-2166 | UGACUCGCCG | 687 | 2146-2166 | AAGACUCGGU | 4164 |
| | GCCGGCGAG | | | GCCACCGAGU | | | GGCCGGCGAG | |
| | UCA | | | CUU | | | UCG | |
| 686 | GCCGGCGAG | 2262 | 2155-2175 | UAUGCGGUCC | 688 | 2155-2175 | UGGCCGGCGA | 4165 |
| | UCGGACCGC | | | GACUCGCCGG | | | GUCGGACCGC | |
| | AUA | | | CCA | | | AUA | |
| 687 | CGGCGAGUC | 2263 | 2157-2177 | UCUAUGCGGU | 689 | 2157-2177 | GCCGGCGAGU | 4166 |
| | GGACCGCAU | | | CCGACUCGCC | | | CGGACCGCAU | |
| | AGA | | | GGC | | | AGA | |
| 688 | GGCGAGUCG | 2264 | 2158-2178 | UUCUAUGCGG | 690 | 2158-2178 | CCGGCGAGUC | 4167 |
| | GACCGCAUA | | | UCCGACUCGC | | | GGACCGCAUA | |
| | GAA | | | CGG | | | GAC | |
| 689 | GAGUCGGAC | 2265 | 2161-2181 | UUCGUCUAUG | 691 | 2161-2181 | GCGAGUCGGA | 4168 |
| | CGCAUAGAC | | | CGGUCCGACU | | | CCGCAUAGAC | |
| | GAA | | | CGC | | | GAU | |
| 690 | AGUCGGACC | 2266 | 2162-2182 | UAUCGUCUAU | 692 | 2162-2182 | CGAGUCGGAC | 4169 |
| | GCAUAGACG | | | GCGGUCCGAC | | | CGCAUAGACG | |
| | AUA | | | UCG | | | AUG | |
| 691 | GUCGGACCG | 2267 | 2163-2182 | UCAUCGUCUA | 693 | 2163-2182 | GAGUCGGACC | 4170 |
| | CAUAGACGA | | | UGCGGUCCGA | | | GCAUAGACGA | |
| | UGA | | | CUC | | | UGG | |
| 692 | GAGACUCCA | 2268 | 2414-2434 | UAGGCGAUUG | 694 | 2414-2434 | CGGAGACUCC | 4171 |
| | GACAAUCGC | | | UCUGGAGUCU | | | AGACAAUCGC | |
| | CUA | | | CCG | | | CUU | |
| 693 | GCCUUUUGC | 2269 | 2430-2450 | UCCGACGAGG | 695 | 2430-2450 | UCGCCUUUUG | 4172 |
| | CUCCUCGUC | | | AGGCAAAAGG | | | CCUCCUCGUC | |
| | GGA | | | CGA | | | GGA | |
| 694 | CUCGUCGGA | 2270 | 2442-2462 | UCCGAGGAGU | 696 | 2442-2462 | UCCUCGUCGG | 4173 |
| | GCACUCCUC | | | GCUCCGACGA | | | AGCACUCCUC | |
| | GGA | | | GGA | | | GGA | |
| 695 | CGUCGGAGC | 2271 | 2444-2464 | UCUCCGAGGA | 697 | 2442-2464 | CUCGUCGGAG | 4174 |
| | ACUCCUCGG | | | GUGCUCCGAC | | | CACUCCUCGG | |
| | AGA | | | GAG | | | AGA | |
| 696 | GAGCACUCC | 2272 | 2449-2469 | UCCGUUCUCC | 698 | 2447-2469 | CGGAGCACUC | 4175 |
| | UCGGAGAAC | | | GAGGAGUGCU | | | CUCGGAGAAC | |
| | GGA | | | CCG | | | GGG | |
| 697 | GAGCACGCC | 2273 | 2544-2564 | UCACUAAUAU | 699 | 2542-2564 | GGGAGCACGC | 4176 |
| | CCAUAUUAG | | | GGGGCGUGCU | | | CCCAUAUUAG | |
| | UGA | | | CCC | | | UGG | |
| 698 | AGCACGCCC | 2274 | 2545-2565 | UCCACUAAUA | 700 | 2543-2565 | GGAGCACGCC | 4177 |
| | CAUAUUAGU | | | UGGGGCGUGC | | | CCAUAUUAGU | |
| | GGA | | | UCC | | | GGU | |
| 699 | CACGCCCCA | 2275 | 2547-2567 | UGACCACUAA | 701 | 2545-2567 | AGCACGCCCC | 4178 |
| | UAUUAGUGG | | | UAUGGGGCGU | | | AUAUUAGUGG | |
| | UCA | | | GCU | | | UCC | |
| 700 | CCCAUAUUA | 2276 | 2552-2572 | UGCCCGGACC | 702 | 2550-2572 | GCCCCAUAUU | 4179 |
| | GUGGUCCGG | | | ACUAAUAUGG | | | AGUGGUCCGG | |
| | GCA | | | GGC | | | GCC | |
| 701 | UAUUAGUGG | 2277 | 2556-2576 | UCCGGGCCCG | 703 | 2554-2576 | CAUAUUAGUG | 4180 |
| | UCCGGGCCC | | | GACCACUAAU | | | GUCCGGGCCC | |
| | GGA | | | AUG | | | GGG | |
| 702 | UUUGUAAGA | 2278 | 2795-2815 | UGCUAAAAGG | 704 | 2793-2815 | AAUUUGUAAG | 4181 |
| | UGCCUUUUA | | | CAUCUUACAA | | | AUGCCUUUUA | |
| | GCA | | | AUU | | | GCG | |
| 703 | UUGUAAGAU | 2279 | 2796-2816 | UCGCUAAAAG | 705 | 2794-2816 | AUUUGUAAGA | 4182 |
| | GCCUUUUAG | | | GCAUCUUACA | | | UGCCUUUUAG | |
| | CGA | | | AAU | | | CGU | |
| 704 | UGUAAGAUG | 2280 | 2797-2817 | UACGCUAAAA | 706 | 2795-2817 | UUUGUAAGAU | 4183 |
| | CCUUUUAGC | | | GGCAUCUUAC | | | GCCUUUUAGC | |
| | GUA | | | AAA | | | GUG | |
| 705 | AAGAUGCCU | 2281 | 2799-2819 | UUACACGCUA | 707 | 2797-2819 | GUAAGAUGCC | 4184 |
| | UUUAGCGUG | | | AAAGGCAUCU | | | UUUUAGCGUG | |
| | UAA | | | UAC | | | UAC | |
| 706 | CUUUUAGCG | 2282 | 2807-2827 | UGGUACUGUA | 708 | 2805-2827 | GCCUUUUAGC | 4185 |
| | UGUACAGUA | | | CACGCUAAAA | | | GUGUACAGUA | |
| | CCA | | | GGC | | | CCC | |
| 707 | UUUUAGCGU | 2283 | 2808-2828 | UGGGUACUGU | 709 | 2806-2828 | CCUUUUAGCG | 4186 |
| | GUACAGUAC | | | ACACGCUAAA | | | UGUACAGUAC | |
| | CCA | | | AGG | | | CCU | |
| 708 | UACAGUACC | 2284 | 2818-2838 | UUGUUUCUCC | 710 | 2816-2838 | UGUACAGUAC | 4187 |
| | CUGGAGAAA | | | AGGGUACUGU | | | CCUGGAGAAA | |
| | CAA | | | ACA | | | CAC | |
| 709 | CAGUACCCU | 2285 | 2820-2840 | UUGUGUUUCU | 711 | 2818-2840 | UACAGUACCC | 4188 |
| | GGAGAAACA | | | CCAGGGUACU | | | UGGAGAAACA | |
| | CAA | | | GUA | | | CAU | |
| 710 | AGUACCCUG | 2286 | 2821-2841 | UAUGUGUUUC | 712 | 2819-2841 | ACAGUACCCU | 4189 |
| | GAGAAACAC | | | UCCAGGGUAC | | | GGAGAAACAC | |
| | AUA | | | UGU | | | AUG | |
| 711 | UGGAGAAAC | 2287 | 2828-2848 | UUUUUUUCAU | 713 | 2826-2848 | CCUGGAGAAA | 4190 |
| | ACAUGAAAA | | | GUGUUUCUCC | | | CACAUGAAAA | |
| | AAA | | | AGG | | | AAU | |
| 712 | GGAGAAACA | 2288 | 2829-2849 | UAUUUUUUCA | 714 | 2827-2849 | CUGGAGAAAC | 4191 |
| | CAUGAAAAA | | | UGUGUUUCUC | | | ACAUGAAAAA | |
| | AUA | | | CAG | | | AUG | |
| 713 | AUGGCACAG | 2289 | 2847-2867 | UACACUCGAU | 715 | 2845-2867 | AAAUGGCACA | 4192 |
| | UGAUCGAGU | | | CACUGUGCCA | | | GUGAUCGAGU | |
| | GUA | | | UUU | | | GUU | |
| 714 | AACAGUCUU | 2290 | 3431-3451 | UGGGUUAAUC | 716 | 3431-3451 | AAAACAGUCU | 4193 |
| | UGGAUUAAC | | | CAAAGACUGU | | | UUGGAUUAAC | |
| | CCA | | | UUU | | | CCU | |
| 715 | GGAUUAACC | 2291 | 3442-3462 | UUGUCUAUAG | 717 | 3442-3462 | UUGGAUUAAC | 4194 |
| | CUCUAUAGA | | | AGGGUUAAUC | | | CCUCUAUAGA | |
| | CAA | | | CAA | | | CAG | |
| 716 | AUAGACAGA | 2292 | 3455-3475 | UGUGCUAUCU | 718 | 3455-3475 | CUAUAGACAG | 4195 |
| | AUAGAUAGC | | | AUUCUGUCUA | | | AAUAGAUAGC | |
| | ACA | | | UAG | | | ACU | |
| 717 | UAAUAAACC | 2293 | 3697-3717 | UCUAGUUCCU | 719 | 3697-3717 | GGUAAUAAAC | 4196 |
| | UUAGGAACU | | | AAGGUUUAUU | | | CUUAGGAACU | |
| | AGA | | | ACC | | | AGC | |
| 718 | AUGACCCAU | 2294 | 3764-3784 | UACUAGGACA | 720 | 3764-3784 | AGAUGACCCA | 4197 |
| | UAUGUCCUA | | | UAAUGGGUCA | | | UUAUGUCCUA | |
| | GUA | | | UCU | | | GUU | |
| 719 | UAUACCAGU | 2295 | 3829-3849 | UGUAGCUUUU | 721 | 3829-3849 | AUUAUACCAG | 4198 |
| | AUAAAAGCU | | | AUACUGGUAU | | | UAUAAAAGCU | |
| | ACA | | | AAU | | | ACU | |
| 720 | AAAGCCCUG | 2296 | 3941-3961 | UUAAUUGCGU | 722 | 3941-3961 | CAAAAGCCCU | 4199 |
| | GAACGCAAU | | | UCCAGGGCUU | | | GGAACGCAAU | |
| | UAA | | | UUG | | | UAA | |
| 721 | AAUUAAAUA | 2297 | 3956-3976 | UCCUUACUAG | 723 | 3956-3961 | GCAAUUAAAU | 4200 |
| | CACUAGUAA | | | UGUAUUUAAU | | | ACACUAGUAA | |
| | GGA | | | UGC | | | GGA | |
| 722 | AUUAAAUAC | 2298 | 3957-3977 | UUCCUUACUA | 724 | 3957-3977 | CAAUUAAAUA | 4201 |
| | ACUAGUAAG | | | GUGUAUUUAA | | | CACUAGUAAG | |
| | GAA | | | UUG | | | GAG | |
| 723 | UAUGAUUUA | 2299 | 4162-4182 | UCGUUGUGCU | 725 | 4162-4182 | AAUAUGAUUU | 4202 |
| | UUAGCACAA | | | AAUAAAUCAU | | | AUUAGCACAA | |
| | CGA | | | AUU | | | CGU | |
| 724 | AUGAUUUAU | 2300 | 4163-4183 | UACGUUGUGC | 726 | 4163-4183 | AUAUGAUUUA | 4203 |
| | UAGCACAAC | | | UAAUAAAUCA | | | UUAGCACAAC | |
| | GUA | | | UAU | | | GUG | |
| 725 | GAUUUAUUA | 2301 | 4165-4185 | UCCACGUUGU | 727 | 4165-4185 | AUGAUUUAUU | 4204 |
| | GCACAACGU | | | GCUAAUAAAU | | | AGCACAACGU | |
| | GGA | | | CAU | | | GGU | |
| 726 | AUUUAUUAG | 2302 | 4166-4186 | UACCACGUUG | 728 | 4166-4186 | UGAUUUAUUA | 4205 |
| | CACAACGUG | | | UGCUAAUAAA | | | GCACAACGUG | |
| | GUA | | | UCA | | | GUA | |
| 727 | UUAUUAGCA | 2303 | 4168-4188 | UGUACCACGU | 729 | 4168-4188 | AUUUAUUAGC | 4206 |
| | CAACGUGGU | | | UGUGCUAAUA | | | ACAACGUGGU | |
| | ACA | | | AAU | | | ACU | |
| 728 | UAGCACAAC | 2304 | 4172-4192 | UAAUAGUACC | 730 | 4172-4192 | AUUAGCACAA | 4207 |
| | GUGGUACUA | | | ACGUUGUGCU | | | CGUGGUACUA | |
| | UUA | | | AAU | | | UUU | |
| 729 | UUUUUUAUU | 2305 | 4438-4458 | UGAUGCUAGC | 731 | 4438-4458 | UCUUUUUUAU | 4208 |
| | AAGCUAGCA | | | UUAAUAAAAA | | | UAAGCUAGCA | |
| | UCA | | | AGA | | | UCU | |
| 730 | UUUAUUAAG | 2306 | 4441-4461 | UGCAGAUGCU | 732 | 4441-4461 | UUUUUAUUAA | 4209 |
| | CUAGCAUCU | | | AGCUUAAUAA | | | GCUAGCAUCU | |
| | GCA | | | AAA | | | GCC | |
| 731 | UUUUCUCUA | 2307 | 4510-4530 | UGUAUCUCUG | 733 | 4510-4530 | UCUUUUCUCU | 4210 |
| | AUCAGAGAU | | | AUUAGAGAAA | | | AAUCAGAGAU | |
| | ACA | | | AGA | | | ACA | |
| 732 | GAUACAGAG | 2308 | 4525-4545 | UUUAUACUCA | 734 | 4523-4545 | GAGAUACAGA | 4211 |
| | GUUGAGUAU | | | ACCUCUGUAU | | | GGUUGAGUAU | |
| | AAA | | | CUC | | | AAA | |
| 733 | CAGAUAGGA | 2309 | 4557-4577 | UGCACUUAAU | 735 | 4555-4577 | CUCAGAUAGG | 4212 |
| | CAAUUAAGU | | | UGUCCUAUCU | | | ACAAUUAAGU | |
| | GCA | | | GAG | | | GCA | |
| 734 | GAUAGGACA | 2310 | 4559-4579 | UGUGCACUUA | 736 | 4557-4579 | CAGAUAGGAC | 4213 |
| | AUUAAGUGC | | | AUUGUCCUAU | | | AAUUAAGUGC | |
| | ACA | | | CUG | | | ACU | |
| 735 | CCCAGUUUA | 2311 | 4590-4610 | UGUAUAGACC | 737 | 4588-4610 | UUCCCAGUUU | 4214 |
| | CAGGUCUAU | | | UGUAAACUGG | | | ACAGGUCUAU | |
| | ACA | | | GAA | | | ACU | |
| 736 | UUGAGCUUA | 2312 | 4719-4739 | UGUCCAAGUA | 738 | 4717-4739 | UAUUGAGCUU | 4215 |
| | CUUACUUGG | | | AGUAAGCUCA | | | ACUUACUUGG | |
| | ACA | | | AUA | | | ACG | |
| 737 | AGCUUACUU | 2313 | 4722-4742 | UUGCGUCCAA | 739 | 4720-4742 | UGAGCUUACU | 4216 |
| | ACUUGGACG | | | GUAAGUAAGC | | | UACUUGGACG | |
| | CAA | | | UCA | | | CAA | |
| 738 | UGGACGCAA | 2314 | 4734-4754 | UCGCUUGCAA | 740 | 4732-4754 | CUUGGACGCA | 4217 |
| | CAUUGCAAG | | | UGUUGCGUCC | | | ACAUUGCAAG | |
| | CGA | | | AAG | | | CGC | |
| 739 | GGACGCAAC | 2315 | 4735-4755 | UGCGCUUGCA | 741 | 4733-4755 | UUGGACGCAA | 4218 |
| | AUUGCAAGC | | | AUGUUGCGUC | | | CAUUGCAAGC | |
| | GCA | | | CAA | | | GCU | |
| 740 | GACGCAACA | 2316 | 4736-4756 | UAGCGCUUGC | 742 | 4734-4756 | UGGACGCAAC | 4219 |
| | UUGCAAGCG | | | AAUGUUGCGU | | | AUUGCAAGCG | |
| | CUA | | | CCA | | | CUG | |
| 741 | GCAACAUUG | 2317 | 4739-4759 | UCACAGCGCU | 743 | 4737-4759 | ACGCAACAUU | 4220 |
| | CAAGCGCUG | | | UGCAAUGUUG | | | GCAAGCGCUG | |
| | UGA | | | CGU | | | UGA | |
| 742 | AAAUGAAUG | 2318 | 4786-4806 | UGCGAAAGCA | 744 | 4784-4806 | AGAAAUGAAU | 4221 |
| | AUUGCUUUC | | | AUCAUUCAUU | | | GAUUGCUUUC | |
| | GCA | | | UCU | | | GCU | |
| 743 | AAUGAAUGA | 2319 | 4787-4807 | UAGCGAAAGC | 745 | 4785-4807 | GAAAUGAAUG | 4222 |
| | UUGCUUUCG | | | AAUCAUUCAU | | | AUUGCUUUCG | |
| | CUA | | | UUC | | | CUU | |
| 744 | AAUGAUUGC | 2320 | 4791-4811 | UUAGAAGCGA | 746 | 4789-4811 | UGAAUGAUUG | 4223 |
| | UUUCGCUUC | | | AAGCAAUCAU | | | CUUUCGCUUC | |
| | UAA | | | UCA | | | UAC | |
| 745 | AUGAUUGCU | 2321 | 4792-4812 | UGUAGAAGCG | 747 | 4790-4812 | GAAUGAUUGC | 4224 |
| | UUCGCUUCU | | | AAAGCAAUCA | | | UUUCGCUUCU | |
| | ACA | | | UUC | | | ACA | |
| 746 | UGAUUGCUU | 2322 | 4793-4813 | UUGUAGAAGC | 748 | 4791-4813 | AAUGAUUGCU | 4225 |
| | UCGCUUCUA | | | GAAAGCAAUC | | | UUCGCUUCUA | |
| | CAA | | | AUU | | | CAG | |
| 747 | AUUUUAGGG | 2323 | 4907-4927 | UAGACAAUGG | 749 | 4905-4927 | GCAUUUUAGG | 4226 |
| | UUCCAUUGU | | | AACCCUAAAA | | | GUUCCAUUGU | |
| | CUA | | | UGC | | | CUU | |
| 748 | AUUGUCUUG | 2324 | 4920-4940 | UUGUAACACC | 750 | 4918-4940 | CCAUUGUCUU | 4227 |
| | GUGGUGUUA | | | ACCAAGACAA | | | GGUGGUGUUA | |
| | CAA | | | UGG | | | CAA | |
| 749 | UGGUAGUGG | 2325 | 4962-4982 | UGCAUAGAAU | 751 | 4960-4982 | AAUGGUAGUG | 4228 |
| | AAAUUCUAU | | | UUCCACUACC | | | GAAAUUCUAU | |
| | GCA | | | AUU | | | GCC | |
| 750 | ACCGCUCUU | 2326 | 4993-5013 | UUUUACAACC | 752 | 4991-5013 | ACACCGCUCU | 4229 |
| | CAGGUUGUA | | | UGAAGAGCGG | | | UCAGGUUGUA | |
| | AAA | | | UGU | | | AAA | |
| 751 | CAUCUAGUG | 2327 | 5155-5175 | UAUAGUGCCU | 753 | 5153-5175 | UUCAUCUAGU | 4230 |
| | UUAGGCACU | | | AACACUAGAU | | | GUUAGGCACU | |
| | AUA | | | GAA | | | AUA | |
| 752 | AUCUAGUGU | 2328 | 5156-5176 | UUAUAGUGCC | 754 | 5154-5176 | UCAUCUAGUG | 4231 |
| | UAGGCACUA | | | UAACACUAGA | | | UUAGGCACUA | |
| | UAA | | | UGA | | | UAG | |
| 753 | GUUAGGCAC | 2329 | 5163-5183 | UUAAAUACUA | 755 | 5161-5183 | GUGUUAGGCA | 4232 |
| | UAUAGUAUU | | | UAGUGCCUAA | | | CUAUAGUAUU | |
| | UAA | | | CAC | | | UAU | |
| 754 | UAAAAGGUA | 2330 | 5220-5540 | UAGGUACAUU | 756 | 5218-5540 | CUUAAAAGGU | 4233 |
| | UCAAUGUAC | | | GAUACCUUUU | | | AUCAAUGUAC | |
| | CUA | | | AAG | | | CUU | |
| 755 | CAGGAUAAU | 2331 | 5296-5316 | UAUUUGCACU | 757 | 5294-5316 | GGCAGGAUAA | 4234 |
| | AUAGUGCAA | | | AUAUUAUCCU | | | UAUAGUGCAA | |
| | AUA | | | GCC | | | AUU | |
| 756 | GAGAAGCCA | 2332 | 5375-5395 | UCCGCCAUUA | 758 | 5373-5395 | AUGAGAAGCC | 4235 |
| | UAUAAUGGC | | | UAUGGCUUCU | | | AUAUAAUGGC | |
| | GGA | | | CAU | | | GGU | |
| 757 | GAAGCCAUA | 2333 | 5377-5397 | UAACCGCCAU | 759 | 5375-5397 | GAGAAGCCAU | 4236 |
| | UAAUGGCGG | | | UAUAUGGCUU | | | AUAAUGGCGG | |
| | UUA | | | CUC | | | UUU | |
| 758 | UGCAGUACU | 2334 | 5486-5506 | UUAUUAGCUU | 760 | 5484-5506 | UAUGCAGUAC | 4237 |
| | GCAAGCUAA | | | GCAGUACUGC | | | UGCAAGCUAA | |
| | UAA | | | AUA | | | UAA | |
| 759 | CUGCAAGCU | 2335 | 5493-5513 | UCCAACGUUA | 761 | 5491-5513 | UACUGCAAGC | 4238 |
| | AAUAACGUU | | | UUAGCUUGCA | | | UAAUAACGUU | |
| | GGA | | | GUA | | | GGU | |
| 760 | UGCAAGCUA | 2336 | 5494-5514 | UACCAACGUU | 762 | 5492-5514 | ACUGCAAGCU | 4239 |
| | AUAACGUUG | | | AUUAGCUUGC | | | AAUAACGUUG | |
| | GUA | | | AGU | | | GUU | |
| 761 | GCAAGCUAA | 2337 | 5495-5515 | UAACCAACGU | 763 | 5493-5515 | CUGCAAGCUA | 4240 |
| | UAACGUUGG | | | UAUUAGCUUG | | | AUAACGUUGG | |
| | UUA | | | CAG | | | UUU | |
| 762 | UAAUAACGU | 2338 | 5501-5521 | UAUAACAAAC | 764 | 5499-5521 | GCUAAUAACG | 4241 |
| | UGGUUUGUU | | | CAACGUUAUU | | | UUGGUUUGUU | |
| | AUA | | | AGC | | | AUG | |
| 763 | AGAAAGCCC | 2339 | 5716-5736 | UUUGACUAAA | 765 | 5714-5736 | AGAGAAAGCC | 4242 |
| | GCUUUAGUC | | | GCGGGCUUUC | | | CGCUUUAGUC | |
| | AAA | | | UCU | | | AAU | |
| 764 | GAAAGCCCG | 2340 | 5717-5737 | UAUUGACUAA | 766 | 5715-5737 | GAGAAAGCCC | 4243 |
| | CUUUAGUCA | | | AGCGGGCUUU | | | GCUUUAGUCA | |
| | AUA | | | CUC | | | AUAC | |
| 765 | AAAGCCCGC | 2341 | 5718-5738 | UUAUUGACUA | 767 | 5716-5738 | AGAAAGCCCG | 4244 |
| | UUUAGUCAA | | | AAGCGGGCUU | | | CUUUAGUCAA | |
| | UAA | | | UCU | | | UAC | |
| 766 | GCCCGCUUU | 2342 | 5721-5741 | UAAGUAUUGA | 768 | 5719-5741 | AAGCCCGCUU | 4245 |
| | AGUCAAUAC | | | CUAAAGCGGG | | | UAGUCAAUAC | |
| | UUA | | | CUU | | | UUU | |
| 767 | GUUGUCUGC | 2343 | 6032-6052 | UAACAGGUUA | 769 | 6030-6052 | CAGUUGUCUG | 4246 |
| | AUUAACCUG | | | AUGCAGACAA | | | CAUUAACCUG | |
| | UUA | | | CUG | | | UUC | |
| 768 | GUUCAUACA | 2344 | 6049-6069 | UGACAAAAUG | 770 | 6047-6069 | CUGUUCAUAC | 4247 |
| | CCCAUUUUG | | | GGUGUAUGAA | | | ACCCAUUUUG | |
| | UCA | | | CAG | | | UCC | |
| 769 | UCAUACACC | 2345 | 6051-6071 | UGGGACAAAA | 771 | 6049-6071 | GUUCAUACAC | 4248 |
| | CAUUUUGUC | | | UGGGUGUAUG | | | CCAUUUUGUC | |
| | CCA | | | AAC | | | CCU | |

The nucleotide sequence of exemplary preferred unmodified versions of the dsRNA agents set forth in Table 2 are set forth in Table 3. More specifically, Table 3 sets forth the nucleotide sequence of exemplary sense strands, antisense strands, and dsRNA agent pairs of sense and antisense strands that may be preferred in some embodiments.

TABLE 3
Unmodified Sense and Antisense Strand Sequences
of Exemplary Preferred BCL11A dsRNA Agents.

| | | | | | | | Exemplary | |
|---|---|---|---|---|---|---|---|---|
| dsRNA | Sense | SEQ | | Antisense | SEQ | | mRNA Target | SEQ |
| Agent | Sequence | ID | Range in | Sequence | ID | Range in | Sequence in | ID |
| ID | 5′ to 3′ | NO | NM_022893.4 | 5′ to 3′ | NO | NM_022893.4 | NM_022893.4 | NO |
| 1 | UGGAUUAAG | 1577 | 963-983 | UCUAAGUAGA | 4 | 961-983 | CAUGGAUUAA | 3548 |
| | AAUCUACUU | | | UUCUUAAUCC | | | GAAUCUACUU | |
| | AGA | | | AUG | | | AGA | |
| 2 | CACAAACGG | 1578 | 580-600 | AUUGCAUUGU | 5 | 578-600 | AGCACAAACG | 3549 |
| | AAACAAUGC | | | UUCCGUUUGU | | | GAAACAAUGC | |
| | AAU | | | GCU | | | AAU | |
| 3 | ACAUUCUUA | 1579 | 560-580 | UCUCGAUAAA | 6 | 558-580 | GGACAUUCUU | 3550 |
| | UUUUUAUCG | | | AAUAAGAAUG | | | AUUUUUAUCG | |
| | AGA | | | UCC | | | AGC | |
| 4 | GCAAACAGC | 1580 | 908-928 | AACUGGUGAA | 7 | 906-928 | UUGCAAACAG | 3551 |
| | CAUUCACCA | | | UGGCUGUUUG | | | CCAUUCACCA | |
| | GUU | | | CAA | | | GUG | |
| 5 | AUGUACAAC | 1581 | 897-917 | UGCUGUUUGC | 8 | 895-917 | ACAUGUACAA | 3552 |
| | UUGCAAACA | | | AAGUUGUACA | | | CUUGCAAACA | |
| | GCA | | | UGU | | | GCC | |
| 6 | AACACGCAC | 1582 | 944-964 | AAUGAGUGUU | 9 | 942-964 | GCAACACGCA | 3553 |
| | AGAACACUC | | | CUGUGCGUGU | | | CAGAACACUC | |
| | AUU | | | UGC | | | AUG | |
| 7 | GCUACACAU | 1583 | 890-910 | UGCAAGUUGU | 10 | 888-910 | CAGCUACACA | 3554 |
| | GUACAACUU | | | ACAUGUGUAG | | | UGUACAACUU | |
| | GCA | | | CUG | | | GCA | |
| 8 | CACAUGUAC | 1584 | 894-914 | UGUUUGCAAG | 11 | 892-914 | UACACAUGUA | 3555 |
| | AACUUGCAA | | | UUGUACAUGU | | | CAACUUGCAA | |
| | ACA | | | GUA | | | ACA | |
| 9 | ACUUGCAAA | 1585 | 904-924 | UGUGAAUGGC | 12 | 902-924 | CAACUUGCAA | 3556 |
| | CAGCCAUUC | | | UGUUUGCAAG | | | ACAGCCAUUC | |
| | ACA | | | UUG | | | ACC | |
| 10 | CGCACAGAA | 1586 | 948-968 | UAUCCAUGAG | 13 | 946-968 | CACGCACAGA | 3557 |
| | CACUCAUGG | | | UGUUCUGUGC | | | ACACUCAUGG | |
| | AUA | | | GUG | | | AUU | |
| 11 | AUUCUUAUU | 1587 | 562-582 | UUGCUCGAUA | 14 | 560-582 | ACAUUCUUAU | 3558 |
| | UUUAUCGAG | | | AAAAUAAGAA | | | UUUUAUCGAG | |
| | CAA | | | UGU | | | CAC | |
| 12 | AGUGCAUGG | 1588 | 925-945 | UUGCAAGAGA | 15 | 923-945 | CCAGUGCAUG | 3559 |
| | UUUCUCUUG | | | AACCAUGCAC | | | GUUUCUCUUG | |
| | CAA | | | UGG | | | CAA | |
| 13 | UCAUCUAGA | 1589 | 730-750 | UGGGCAAAUU | 16 | 728-750 | CGUCAUCUAG | 3560 |
| | GGAAUUUGC | | | CCUCUAGAUG | | | AGGAAUUUGC | |
| | CCA | | | ACG | | | CCC | |
| 14 | GGCGAGAAG | 1590 | 2083-2103 | UCCGCGCUUA | 17 | 2081-2103 | UGGGCGAGAA | 3561 |
| | CAUAAGCGC | | | UGCUUCUCGC | | | GCAUAAGCGC | |
| | GGA | | | CCA | | | GGC | |
| 15 | GUAAGAUGC | 1591 | 2798-2818 | UCACGCUAAA | 18 | 2796-2818 | UUGUAAGAUG | 3562 |
| | CUUUUAGCG | | | AGGCAUCUUA | | | CCUUUUAGCG | |
| | UGA | | | CAA | | | UGU | |
| 16 | GGGAUUCAU | 1592 | 1069-1089 | AUUGUCUGCA | 19 | 1067-1089 | AUGGGAUUCA | 3563 |
| | AUUGCAGAC | | | AUAUGAAUCC | | | UAUUGCAGAC | |
| | AAU | | | CAU | | | AAU | |

The nucleotide sequence of exemplary modified versions of the dsRNA agents set forth in Table 2 are set forth in Table 4. More specifically, Table 8 sets forth the nucleotide sequence of exemplary sense strands, antisense strands, and dsRNA agent pairs of sense and antisense strands. It is to be understood that while the sense and antisense strands are set forth in pairs in Table 4, the disclosure encompasses dsRNA agents comprising any sense strand and any antisense set forth in Table 4 (e.g., that are at least partially complementary (e.g., as could be determined by a person of ordinary skill in the art)). It is to be understood that while the nucleotide sequence of the sense strands and antisense strands in Table 4 are set forth as modified (i.e., contain at least one modified nucleotide), the disclosure encompasses the sense and antisense sense strands set forth in Table 4 comprising other nucleotide modifications (e.g., as described herein) or that are unmodified.

TABLE 4
Modified Sense and Antisense Strand Sequences of BCL11A dsRNA Agents.

| | | | | | | | Exemplary | |
|---|---|---|---|---|---|---|---|---|
| dsRNA | Sense | SEQ | | Antisense | SEQ | | mRNA Target | SEQ |
| Agent | Sequence | ID | Range in | Sequence | ID | Range in | Sequence in | ID |
| ID | 5′ to 3′ | NO | NM_022893.4 | 5′ to 3′ | NO | NM_022893.4 | NM_022893.4 | NO |
| 770 | u*g*gauuA | 2346 | 963-983 | (VPmU)*C*uaa | 772 | 961-983 | CAUGGAUUAA | 3548 |
| | aGAAucuaC | | | GuAGauucUuAa | | | GAAUCUACUU | |
| | Uua*g*a | | | (y)cca*u*g | | | AGA | |
| 771 | c*a*caaaC | 2347 | 580-600 | (VPmA)*U*ugc | 773 | 578-600 | AGCACAAACG | 3549 |
| | gGAAacaaU | | | AuUGuuucCgUu | | | GAAACAAUGC | |
| | Gca*a*u | | | ug(y)g*c*u | | | AAU | |
| 772 | a*c*auucU | 2348 | 560-580 | (VPmU)*C*ucg | 774 | 558-580 | GGACAUUCUU | 3550 |
| | uAUUuuuau | | | AuaaaaauAaGa | | | AUUUUUAUCG | |
| | cga*g*a | | | augu*c*c | | | AGC | |
| 773 | g*c*aaacA | 2349 | 908-928 | (VPmA)*A*cug | 775 | 906-928 | UUGCAAACAG | 3551 |
| | gCCAuucaC | | | GuGAaugXCuGu | | | CCAUUCACCA | |
| | CaX*u*u | | | (y)ugc*a*a | | | GUG | |
| 774 | a*u*guacA | 2350 | 897-917 | (VPmU)*G*cug | 776 | 895-917 | ACAUGUACAA | 3552 |
| | aCUUgcaaA | | | UuUGcaagUuGu | | | CUUGCAAACA | |
| | CaX*c*a | | | acau*g*u | | | GCC | |
| 775 | a*a*cacgC | 2351 | 944-964 | (VPmA)*A*uga | 777 | 942-964 | GCAACACGCA | 3553 |
| | aCAGaacaC | | | GuGUucugUgCg | | | CAGAACACUC | |
| | Uca*u*u | | | uguu*g*c | | | AUG | |
| 776 | g*c*uacaC | 2352 | 890-910 | (VPmU)*G*caa | 778 | 888-910 | CAGCUACACA | 3554 |
| | aUGUacaaC | | | GuUGuacaUgUg | | | UGUACAACUU | |
| | Uug*c*a | | | (y)agc*u*g | | | GCA | |
| 777 | c*a*caugU | 2353 | 894-914 | (VPmU)*G*uuu | 779 | 892-914 | UACACAUGUA | 3555 |
| | aCAAcuugc | | | GcaaguugUaCa | | | CAACUUGCAA | |
| | aaa*c*a | | | ugug*u*a | | | ACA | |
| 778 | a*c*uugcA | 2354 | 904-924 | (VPmU)*G*uga | 780 | 902-924 | CAACUUGCAA | 3556 |
| | aACAgccaU | | | AuGGcuguUuGc | | | ACAGCCAUUC | |
| | Uca*c*a | | | aag(y)*u*g | | | ACC | |
| 779 | c*g*cacaG | 2355 | 948-968 | (VPmU)*A*ucc | 781 | 946-968 | CACGCACAGA | 3557 |
| | aACAcucau | | | AugaguguUcUg | | | ACACUCAUGG | |
| | gga*u*a | | | ugcg*u*g | | | AUU | |
| 780 | a*u*ucuuA | 2356 | 562-582 | (VPmU)*U*gcu | 782 | 560-582 | ACAUUCUUAU | 3558 |
| | uUUUuaucg | | | CgauaaaaAuAa | | | UUUUAUCGAG | |
| | agc*a*a | | | gaau*g*u | | | CAC | |
| 781 | a*g*ugcaU | 2357 | 925-945 | (VPmU)*U*gca | 783 | 923-945 | CCAGUGCAUG | 3559 |
| | gGUUucucU | | | AgAGaaacCaUg | | | GUUUCUCUUG | |
| | UXc*a*a | | | cac(y)*g*g | | | CAA | |
| 782 | u*c*aucuA | 2358 | 730-750 | (VPmU)*G*ggc | 784 | 728-750 | CGUCAUCUAG | 3560 |
| | gAGGaauuu | | | AaauuccuCuAg | | | AGGAAUUUGC | |
| | gcc*c*a | | | auga*c*g | | | CCC | |
| 783 | g*g*cgagA | 2359 | 2083-2103 | (VPmU)*C*cgc | 785 | 2081-2103 | UGGGCGAGAA | 3561 |
| | aGCAuaagc | | | GcuuaugcUuCu | | | GCAUAAGCGC | |
| | gcg*g*a | | | cgcc*c*a | | | GGC | |
| 784 | g*u*aagaU | 2360 | 2798-2818 | (VPmU)*C*acg | 786 | 2796-2818 | UUGUAAGAUG | 3562 |
| | gCCUuuuag | | | CuaaaaggCaUc | | | CCUUUUAGCG | |
| | cgu*g*a | | | uuac*a*a | | | UGU | |
| 785 | g*g*gauuC | 2361 | 1069-1089 | (VPmA)*U*ugu | 787 | 1067-1089 | AUGGGAUUCA | 3563 |
| | aUAUugcaG | | | CuGCaauaUgAa | | | UAUUGCAGAC | |
| | Aca*a*u | | | (y)ccc*a*u | | | AAU | |
| 786 | a*u*UaAgA | 2362 | 966-986 | (VPmU)*U*uUc | 788 | 964-986 | GGAUUAAGAA | 3564 |
| | aUcUaCuUa | | | UaAgUaGaUuCu | | | UCUACUUAGA | |
| | GaA*a*g | | | UaAu*c*c | | | AAG | |
| 787 | g*a*AgGaC | 2363 | 2772-2792 | (VPmU)*C*aCa | 789 | 2770-2792 | GGGAAGGACG | 3565 |
| | gUuUaCaAa | | | UuUgUaAaCgUc | | | UUUACAAAUG | |
| | UgU*g*a | | | CuUc*c*c | | | UGA | |
| 788 | g*c*AcAgA | 2364 | 949-969 | (VPmU)*A*aUc | 790 | 947-969 | ACGCACAGAA | 3566 |
| | aCaCuCaUg | | | CaUgAgUgUuCu | | | CACUCAUGGA | |
| | GaU*u*a | | | GuGc*g*u | | | UUA | |
| 789 | g*g*AcUaG | 2365 | 1030-1050 | (VPmA)*G*gAc | 791 | 1028-1050 | CAGGACUAGG | 3567 |
| | gUgCaGaAu | | | AuUcUgCaCcUa | | | UGCAGAAUGU | |
| | GuC*c*u | | | GuCc*u*g | | | CCU | |
| 790 | g*u*GaAaU | 2366 | 2789-2809 | (VPmA)*A*gGc | 792 | 2787-2809 | AUGUGAAAUU | 3568 |
| | uUgUaAgAu | | | AuCuUaCaAaUu | | | UGUAAGAUGC | |
| | GcC*u*u | | | UcAc*a*u | | | CUU | |
| 791 | a*c*GuUcA | 2367 | 1537-1557 | (VPmG)*U*uGc | 793 | 1535-1557 | AGACGUUCAA | 3569 |
| | aAuUuCaGa | | | UcUgAaAuUuGa | | | AUUUCAGAGC | |
| | GcA*a*c | | | AcGu*c*u | | | AAC | |
| 792 | c*a*CuCaU | 2368 | 957-977 | (VPmU)*A*gAu | 794 | 955-977 | AACACUCAUG | 3570 |
| | gGaUuAaGa | | | UcUuAaUcCaUg | | | GAUUAAGAAU | |
| | AuC*u*a | | | AgUg*u*u | | | CUA | |
| 793 | c*a*AcGuC | 2369 | 725-745 | (VPmA)*A*aUu | 795 | 723-745 | AUCAACGUCA | 3571 |
| | aUcUaGaGg | | | CcUcUaGaUgAc | | | UCUAGAGGAA | |
| | AaU*u*u | | | GuUg*a*u | | | UUU | |
| 794 | c*u*CuAgG | 2370 | 1329-1349 | (VPmA)*G*cUc | 796 | 1327-1349 | UUCUCUAGGA | 3572 |
| | aGaCuUaGa | | | UcUaAgUcUcCu | | | GACUUAGAGA | |
| | GaG*c*u | | | AgAg*a*a | | | GCU | |
| 795 | a*c*AgAaC | 2371 | 951-971 | (VPmU)*U*uAa | 797 | 949-971 | GCACAGAACA | 3573 |
| | aCuCaUgGa | | | UcCaUgAgUgUu | | | CUCAUGGAUU | |
| | UuA*a*g | | | CuGu*g*c | | | AAG | |
| 796 | g*c*UuCgG | 2372 | 2408-2428 | (VPmA)*U*uGu | 798 | 2406-2428 | UAGCUUCGGA | 3574 |
| | aGaCuCcAg | | | CuGgAgUcUcCg | | | GACUCCAGAC | |
| | AcA*a*u | | | AaGc*u*a | | | AAU | |
| 797 | a*a*CaCuC | 2373 | 955-975 | (VPmG)*A*uUc | 799 | 953-975 | AGAACACUCA | 3575 |
| | aUgGaUuAa | | | UuAaUcCaUgAg | | | UGGAUUAAGA | |
| | GaA*u*c | | | UgUu*c*u | | | AUC | |
| 798 | u*g*AcAgG | 2374 | 1275-1295 | (VPmU)*U*cAa | 800 | 1273-1295 | UUUGACAGGG | 3576 |
| | gUgCuGcGg | | | CcGcAgCaCcCu | | | UGCUGCGGUU | |
| | UuG*a*a | | | GuCa*a*a | | | GAA | |
| 799 | u*c*GaGuG | 2375 | 2859-2879 | (VPmA)*U*aUc | 801 | 2857-2879 | GAUCGAGUGU | 3577 |
| | uUgAaUaAu | | | AuUaUuCaAcAc | | | UGAAUAAUGA | |
| | GaU*a*u | | | UcGa*u*c | | | UAU | |
| 800 | a*u*GaCgA | 2376 | 710-730 | (VPmA)*C*gUu | 802 | 708-730 | GGAUGACGAU | 3578 |
| | uUgUuUaUc | | | GaUaAaCaAuCg | | | UGUUUAUCAA | |
| | AaC*g*u | | | UcAu*c*c | | | CGU | |
| 801 | a*u*GgAuU | 2377 | 1321-1341 | (VPmA)*A*gUc | 803 | 1319-1341 | CCAUGGAUUU | 3579 |
| | uCuCuAgGa | | | UcCuAgAgAaAu | | | CUCUAGGAGA | |
| | GaC*u*u | | | CcAu*g*g | | | CUU | |
| 802 | u*g*UgAgU | 2378 | 2614-2634 | (VPmG)*A*cUu | 804 | 2612-2634 | CUUGUGAGUA | 3580 |
| | aCuGuGgGa | | | UcCcAcAgUaCu | | | CUGUGGGAAA | |
| | AaG*u*c | | | CaCa*a*g | | | GUC | |
| 803 | c*u*CcAgG | 2379 | 2379-2399 | (VPmG)*G*aUc | 805 | 2377-2399 | GCCUCCAGGC | 3581 |
| | cAgCuCaAa | | | UuUgAgCuGcCu | | | AGCUCAAAGA | |
| | GaU*c*c | | | GgAg*g*c | | | UCC | |
| 804 | g*c*AgCgA | 2380 | 2603-2623 | (VPmA)*G*uAc | 806 | 2601-2623 | ACGCAGCGAC | 3582 |
| | cAcUuGuGa | | | UcAcAaGuGuCg | | | ACUUGUGAGU | |
| | GuA*c*u | | | CuGc*g*u | | | ACU | |
| 805 | c*u*GuAgC | 2381 | 2643-2663 | (VPmU)*G*gAc | 807 | 2641-2663 | AACUGUAGCA | 3583 |
| | aAuCuCaCu | | | AgUgAgAuUgCu | | | AUCUCACUGU | |
| | GuC*c*a | | | AcAg*u*u | | | CCA | |
| 806 | u*c*UuCaA | 2382 | 2633-2653 | (VPmG)*A*uUg | 808 | 2631-2653 | AGUCUUCAAG | 3584 |
| | gAaCuGuAg | | | CuAcAgUuCuUg | | | AACUGUAGCA | |
| | CaA*u*c | | | AaGa*c*u | | | AUC | |
| 807 | c*u*GcUaA | 2383 | 1102-1122 | (VPmU)*G*aUc | 809 | 1100-1122 | ACCUGCUAAG | 3585 |
| | gAaUaCcAg | | | CuGgUaUuCuUa | | | AAUACCAGGA | |
| | GaU*c*a | | | GcAg*g*u | | | UCA | |
| 808 | g*g*UuGgU | 2384 | 1014-1034 | (VPmA)*G*uCc | 810 | 1012-1034 | CGGGUUGGUA | 3586 |
| | aUcCcUuCa | | | UgAaGgGaUaCc | | | UCCCUUCAGG | |
| | GgA*c*u | | | AaCc*c*g | | | ACU | |
| 809 | c*g*CaGcG | 2385 | 2602-2622 | (VPmG)*U*aCu | 811 | 2600-2622 | GACGCAGCGA | 3587 |
| | aCaCuUgUg | | | CaCaAgUgUcGc | | | CACUUGUGAG | |
| | AgU*a*c | | | UgCg*u*c | | | UAC | |
| 810 | c*g*AgCaC | 2386 | 576-596 | (VPmU)*A*uUg | 812 | 574-596 | AUCGAGCACA | 3588 |
| | aAaCgGaAa | | | UuUcCgUuUgUg | | | AACGGAAACA | |
| | CaA*u*g | | | CuCg*a*u | | | AUG | |
| 811 | u*c*AaCgU | 2387 | 724-744 | (VPmA)*A*uUc | 813 | 722-744 | UAUCAACGUC | 3589 |
| | cAuCuAgAg | | | CuCuAgAuGaCg | | | AUCUAGAGGA | |
| | GaA*u*u | | | UuGa*u*a | | | AUU | |
| 812 | u*g*CgGuU | 2388 | 1286-1306 | (VPmU)*A*gCc | 814 | 1284-1306 | GCUGCGGUUG | 3590 |
| | gAaUcCaAu | | | AuUgGaUuCaAc | | | AAUCCAAUGG | |
| | GgC*u*a | | | CgCa*g*c | | | CUA | |
| 813 | g*a*CgAuU | 2389 | 712-732 | (VPmU)*G*aCg | 815 | 710-732 | AUGACGAUUG | 3591 |
| | gUuUaUcAa | | | UuGaUaAaCaAu | | | UUUAUCAACG | |
| | CgU*c*a | | | CgUc*a*u | | | UCA | |
| 814 | u*g*GaGuC | 2390 | 284-304 | (VPmG)*U*uAg | 816 | 282-304 | UCUGGAGUCU | 3592 |
| | uCcUuCuUu | | | AaAgAaGgAgAc | | | CCUUCUUUCU | |
| | CuA*a*c | | | UcCa*g*a | | | AAC | |
| 815 | c*g*CaUaG | 2391 | 2170-2190 | (VPmA)*A*cAg | 817 | 2168-2190 | ACCGCAUAGA | 3593 |
| | aCgAuGgCa | | | UgCcAuCgUcUa | | | CGAUGGCACU | |
| | CuG*u*u | | | UgCg*g*u | | | GUU | |
| 816 | u*g*UgAaA | 2392 | 2788-2808 | (VPmA)*G*gCa | 818 | 2786-2808 | AAUGUGAAAU | 3594 |
| | uUuGuAaGa | | | UcUuAcAaAuUu | | | UUGUAAGAUG | |
| | UgC*c*u | | | CaCa*u*u | | | CCU | |
| 817 | c*a*GaAcA | 2393 | 952-972 | (VPmU)*C*uUa | 819 | 950-972 | CACAGAACAC | 3595 |
| | cUcAuGgAu | | | AuCcAuGaGuGu | | | UCAUGGAUUA | |
| | UaA*g*a | | | UcUg*u*g | | | AGA | |
| 818 | a*c*GgCuC | 2394 | 191-211 | (VPmG)*A*uGa | 820 | 189-211 | UGACGGCUCU | 3596 |
| | uCcCaCaAu | | | AuUgUgGgAgAg | | | CCCACAAUUC | |
| | UcA*u*c | | | CcGu*c*a | | | AUC | |
| 819 | g*g*AaAgU | 2395 | 2627-2647 | (VPmU)*A*cAg | 821 | 2625-2647 | UGGGAAAGUC | 3597 |
| | cUuCaAgAa | | | UuCuUgAaGaCu | | | UUCAAGAACU | |
| | CuG*u*a | | | UuCc*c*a | | | GUA | |
| 820 | c*u*UaUaA | 2396 | 2690-2710 | (VPmU)*G*cAc | 822 | 2688-2710 | GCCUUAUAAA | 3598 |
| | aUgCgAgCu | | | AgCuCgCaUuUa | | | UGCGAGCUGU | |
| | GuG*c*a | | | UaAg*g*c | | | GCA | |
| 821 | u*u*CaAgA | 2397 | 2635-2655 | (VPmG)*A*gAu | 823 | 2633-2655 | UCUUCAAGAA | 3599 |
| | aCuGuAgCa | | | UgCuAcAgUuCu | | | CUGUAGCAAU | |
| | AuC*u*c | | | UgAa*g*a | | | CUC | |
| 822 | u*c*CaGgC | 2398 | 2380-2400 | (VPmG)*G*gAu | 824 | 2378-2400 | CCUCCAGGCA | 3600 |
| | aGcUcAaAg | | | CuUuGaGcUgCc | | | GCUCAAAGAU | |
| | AuC*c*c | | | UgGa*g*g | | | CCC | |
| 823 | g*g*AuGaC | 2399 | 708-728 | (VPmG)*U*uGa | 825 | 706-728 | GAGGAUGACG | 3601 |
| | gAuUgUuUa | | | UaAaCaAuCgUc | | | AUUGUUUAUC | |
| | UcA*a*c | | | AuCc*u*c | | | AAC | |
| 824 | c*u*GcGgU | 2400 | 1285-1305 | (VPmA)*G*cCa | 826 | 1283-1305 | UGCUGCGGUU | 3602 |
| | uGaAuCcAa | | | UuGgAuUcAaCc | | | GAAUCCAAUG | |
| | UgG*c*u | | | GcAg*c*a | | | GCU | |
| 825 | u*g*AcGaU | 2401 | 711-731 | (VPmG)*A*cGu | 827 | 709-731 | GAUGACGAUU | 3603 |
| | uGuUuAuCa | | | UgAuAaAcAaUc | | | GUUUAUCAAC | |
| | AcG*u*c | | | GuCa*u*c | | | GUC | |
| 826 | c*u*UcGgA | 2402 | 2409-2429 | (VPmG)*A*uUg | 828 | 2407-2429 | AGCUUCGGAG | 3604 |
| | gAcUcCaGa | | | UcUgGaGuCuCc | | | ACUCCAGACA | |
| | CaA*u*c | | | GaAg*c*u | | | AUC | |
| 827 | c*u*UcAaG | 2403 | 2634-2654 | (VPmA)*G*aUu | 829 | 2632-2654 | GUCUUCAAGA | 3605 |
| | aAcUgUaGc | | | GcUaCaGuUcUu | | | ACUGUAGCAA | |
| | AaU*c*u | | | GaAg*a*c | | | UCU | |
| 828 | g*c*GaGcU | 2404 | 2699-2719 | (VPmA)*G*gCa | 830 | 2697-2719 | AUGCGAGCUG | 3606 |
| | gUgCaAcUa | | | UaGuUgCaCaGc | | | UGCAACUAUG | |
| | UgC*c*u | | | UcGc*a*u | | | CCU | |
| 829 | a*u*CcAaU | 2405 | 1295-1315 | (VPmG)*A*gGc | 831 | 1293-1315 | GAAUCCAAUG | 3607 |
| | gGcUaUgGa | | | UcCaUaGcCaUu | | | GCUAUGGAGC | |
| | GcC*u*c | | | GgAu*u*c | | | CUC | |
| 830 | g*c*CuUaU | 2406 | 2688-2708 | (VPmU)*A*cAg | 832 | 2686-2708 | AGGCCUUAUA | 3608 |
| | aAaUgCgAg | | | CuCgCaUuUaUa | | | AAUGCGAGCU | |
| | CuG*u*g | | | AgGc*c*u | | | GUG | |
| 831 | c*a*CuUaA | 2407 | 415-435 | (VPmG)*A*aUu | 833 | 413-435 | AGCACUUAAG | 3609 |
| | gCaAaCgGg | | | CcCgUuUgCuUa | | | CAAACGGGAA | |
| | AaU*u*c | | | AgUg*c*u | | | UUC | |
| 832 | c*a*AgUcC | 2408 | 1506-1526 | (VPmA)*A*cUc | 834 | 1504-1526 | GUCAAGUCCA | 3610 |
| | aAgUcAuGc | | | GcAuGaCuUgGa | | | AGUCAUGCGA | |
| | GaG*u*u | | | CuUg*a*c | | | GUU | |
| 833 | g*g*AuUaA | 2409 | 964-984 | (VPmU)*U*cUa | 835 | 962-984 | AUGGAUUAAG | 3611 |
| | gAaUcUaCu | | | AgUaGaUuCuUa | | | AAUCUACUUA | |
| | UaG*a*a | | | AuCc*a*u | | | GAA | |
| 834 | c*a*AuCuC | 2410 | 2649-2669 | (VPmU)*U*cCu | 836 | 2647-2669 | AGCAAUCUCA | 3612 |
| | aCuGuCcAc | | | GuGgAcAgUgAg | | | CUGUCCACAG | |
| | AgG*a*g | | | AuUg*c*u | | | GAG | |
| 835 | a*a*CcCgG | 2411 | 302-322 | (VPmU)*C*aCa | 837 | 300-322 | CUAACCCGGC | 3613 |
| | CUcUcCcGa | | | UcGgGaGaGcCg | | | UCUCCCGAUG | |
| | UgU*g*a | | | GgUu*a*g | | | UGA | |
| 836 | a*c*AcUcA | 2412 | 956-976 | (VPmA)*G*aUu | 838 | 954-976 | GAACACUCAU | 3553 |
| | uGgAuUaAg | | | CuUaAuCcAuGa | | | GGAUUAAGAA | |
| | AaU*c*u | | | GuGu*u*c | | | UCU | |
| 837 | c*a*UgAcG | 2413 | 187-207 | (VPmA)*A*uUg | 839 | 185-207 | GCCAUGACGG | 3554 |
| | gCuCuCcCa | | | UgGgAgAgCcGu | | | CUCUCCCACA | |
| | CaA*u*u | | | CaUg*g*c | | | AUU | |
| 838 | g*g*AcGuU | 2414 | 2775-2795 | (VPmA)*U*uUc | 840 | 2773-2795 | AAGGACGUUU | 3555 |
| | uAcAaAuGu | | | AcAuUuGuAaAc | | | ACAAAUGUGA | |
| | GaA*a*u | | | GuCc*u*u | | | AAU | |
| 839 | u*a*AgAaU | 2415 | 1106-1126 | (VPmA)*U*aCu | 841 | 1104-1126 | GCUAAGAAUA | 3556 |
| | aCcAgGaUc | | | GaUcCuGgUaUu | | | CCAGGAUCAG | |
| | AgU*a*u | | | CuUa*g*c | | | UAU | |
| 840 | a*u*GaCgG | 2416 | 188-208 | (VPmG)*A*aUu | 842 | 186-208 | CCAUGACGGC | 3557 |
| | cUcUcCcAc | | | GuGgGaGaGcCg | | | UCUCCCACAA | |
| | AaU*u*c | | | UcAu*g*g | | | UUC | |
| 841 | a*g*CaCaA | 2417 | 578-598 | (VPmU)*G*cAu | 843 | 576-598 | CGAGCACAAA | 3558 |
| | aCgGaAaCa | | | UgUuUcCgUuUg | | | CGGAAACAAU | |
| | AuG*c*a | | | UgCu*c*g | | | GCA | |
| 842 | g*u*UuCuC | 2418 | 933-953 | (VPmU)*G*uGc | 844 | 931-953 | UGGUUUCUCU | 3559 |
| | uUgCaAcAc | | | GuGuUgCaAgAg | | | UGCAACACGC | |
| | GcA*c*a | | | AaAc*c*a | | | ACA | |
| 843 | c*u*GuGcA | 2419 | 2704-2724 | (VPmG)*G*cAc | 845 | 2702-2724 | AGCUGUGCAA | 3560 |
| | aCuAuGcCu | | | AgGcAuAgUuGc | | | CUAUGCCUGU | |
| | GuG*c*c | | | AcAg*c*u | | | GCC | |
| 844 | u*g*CaAaG | 2420 | 1400-1420 | (VPmA)*U*gGu | 846 | 1398-1420 | UAUGCAAAGG | 3561 |
| | gUuAcUgCa | | | UgCaGuAaCcUu | | | UUACUGCAAC | |
| | AcC*a*u | | | UgCa*u*a | | | CAU | |
| 845 | u*c*GcCgC | 2421 | 390-410 | (VPmG)*G*uUu | 847 | 388-410 | UCUCGCCGCA | 3562 |
| | aAgCaAgGc | | | GcCuUgCuUgCg | | | AGCAAGGCAA | |
| | AaA*c*c | | | GcGa*g*a | | | ACC | |
| 846 | c*c*UuCaG | 2422 | 1024-1044 | (VPmU)*U*cUg | 848 | 1022-1044 | UCCCUUCAGG | 3563 |
| | gAcUaGgUg | | | CaCcUaGuCcUg | | | ACUAGGUGCA | |
| | CaG*a*a | | | AaGg*g*a | | | GAA | |
| 847 | u*c*UuGcA | 2423 | 938-958 | (VPmU)*G*uUc | 849 | 936-958 | UCUCUUGCAA | 3564 |
| | aCaCgCaCa | | | UgUgCgUgUuGc | | | CACGCACAGA | |
| | GaA*c*a | | | AaGa*g*a | | | ACA | |
| 848 | a*c*GuCaU | 2424 | 727-747 | (VPmG)*C*aAa | 850 | 725-747 | CAACGUCAUC | 3565 |
| | cUaGaGgAa | | | UuCcUcUaGaUg | | | UAGAGGAAUU | |
| | UuU*g*c | | | AcGu*u*g | | | UGC | |
| 849 | c*a*CaGaA | 2425 | 950-970 | (VPmU)*U*aAu | 851 | 948-970 | CGCACAGAAC | 3566 |
| | cAcUcAuGg | | | CcAuGaGuGuUc | | | ACUCAUGGAU | |
| | AuU*a*a | | | UgUg*c*g | | | UAA | |
| 850 | u*c*UgGaG | 2426 | 282-302 | (VPmU)*A*gAa | 852 | 280-302 | UCUCUGGAGU | 3567 |
| | uCuCcUuCu | | | AgAaGgAgAcUc | | | CUCCUUCUUU | |
| | UuC*u*a | | | CaGa*g*a | | | CUA | |
| 851 | u*a*AcCcG | 2427 | 301-321 | (VPmU)*A*cAu | 853 | 299-321 | UCUAACCCGG | 3568 |
| | gCuCuCcCg | | | CgGgAgAgCcGg | | | CUCUCCCGAU | |
| | AuG*u*g | | | GuUa*g*a | | | GUG | |
| 852 | a*g*CaCuU | 2428 | 413-433 | (VPmA)*U*uCc | 854 | 411-433 | CCAGCACUUA | 3569 |
| | aAgCaAaCg | | | CgUuUgCuUaAg | | | AGCAAACGGG | |
| | GgA*a*u | | | UgCu*g*g | | | AAU | |
| 853 | a*a*AcGgA | 2429 | 583-603 | (VPmG)*C*cAu | 855 | 581-603 | ACAAACGGAA | 3570 |
| | aAcAaUgCa | | | UgCaUuGuUuCc | | | ACAAUGCAAU | |
| | AuG*g*c | | | GuUu*g*u | | | GGC | |
| 854 | a*u*GgAuU | 2430 | 962-982 | (VPmU)*U*aAg | 856 | 960-982 | UCAUGGAUUA | 3571 |
| | aAgAaUcUa | | | UaGaUuCuUaAu | | | AGAAUCUACU | |
| | CuU*a*g | | | CcAu*g*a | | | UAG | |
| 855 | c*c*UgCuA | 2431 | 1101-1121 | (VPmG)*A*uCc | 857 | 1099-1121 | AACCUGCUAA | 3572 |
| | aGaAuAcCa | | | UgGuAuUcUuAg | | | GAAUACCAGG | |
| | GgA*u*c | | | CaGg*u*u | | | AUC | |
| 856 | a*c*GuCcG | 2432 | 47-67 | (VPmU)*G*cAa | 858 | 45-67 | UCACGUCCGC | 3573 |
| | cAcUuGaAc | | | GuUcAaGuGcGg | | | ACUUGAACUU | |
| | UuG*c*a | | | AcGu*g*a | | | GCA | |
| 857 | c*a*CaUaG | 2433 | 760-780 | (VPmU)*A*gAa | 859 | 758-780 | AACACAUAGC | 3574 |
| | cAgAuAaAc | | | GuUuAuCuGcUa | | | AGAUAAACUU | |
| | UuC*u*g | | | UgUg*u*u | | | CUG | |
| 858 | u*g*CuCaG | 2434 | 2036-2056 | (VPmA)*G*uGc | 860 | 2034-2056 | GGUGCUCAGC | 3575 |
| | cUcCaUgCa | | | UgCaUgGaGcUg | | | UCCAUGCAGC | |
| | GcA*c*u | | | AgCa*c*c | | | ACU | |
| 859 | a*c*AuAgC | 2435 | 761-781 | (VPmG)*C*aGa | 861 | 759-781 | ACACAUAGCA | 3576 |
| | aGaUaAaCu | | | AgUuUaUcUgCu | | | GAUAAACUUC | |
| | UcU*g*c | | | AuGu*g*u | | | UGC | |
| 860 | a*a*CaCgC | 2436 | 944-964 | (VPmU)*A*uGa | 862 | 942-964 | GCAACACGCA | 3577 |
| | aCaGaAcAc | | | GuGuUcUgUgCg | | | CAGAACACUC | |
| | UcA*u*g | | | UgUu*g*c | | | AUG | |
| 861 | g*c*UgCgG | 2437 | 1284-1304 | (VPmG)*C*cAu | 863 | 1282-1304 | GUGCUGCGGU | 3578 |
| | uUgAaUcCa | | | UgGaUuCaAcCg | | | UGAAUCCAAU | |
| | AuG*g*c | | | CaGc*a*c | | | GGC | |
| 862 | u*c*CgCaC | 2438 | 50-70 | (VPmA)*G*cUg | 864 | 48-70 | CGUCCGCACU | 3579 |
| | uUgAaCuUg | | | CaAgUuCaAgUg | | | UGAACUUGCA | |
| | CaG*c*u | | | CgGa*c*g | | | GCU | |
| 863 | g*u*UgAaU | 2439 | 1290-1310 | (VPmU)*C*cAu | 865 | 1288-1310 | CGGUUGAAUC | 3580 |
| | cCaAuGgCu | | | AgCcAuUgGaUu | | | CAAUGGCUAU | |
| | AuG*g*a | | | CaAc*c*g | | | GGA | |
| 864 | a*a*CuGuA | 2440 | 2641-2661 | (VPmG)*A*cAg | 866 | 2639-2661 | AGAACUGUAG | 3581 |
| | gCaAuCuCa | | | UgAgAuUgCuAc | | | CAAUCUCACU | |
| | CuG*u*c | | | AgUu*c*u | | | GUC | |
| 865 | c*c*GgCuC | 2441 | 305-325 | (VPmG)*G*uUc | 867 | 303-325 | ACCCGGCUCU | 3582 |
| | uCcCgAuGu | | | AcAuCgGgAgAg | | | CCCGAUGUGA | |
| | GaA*c*c | | | CcGg*g*u | | | ACC | |
| 866 | c*u*UcCcU | 2442 | 211-231 | (VPmU)*A*cAa | 868 | 209-231 | AUCUUCCCUG | 3583 |
| | gCgCcAuCu | | | AgAuGgCgCaGg | | | CGCCAUCUUU | |
| | UuG*u*a | | | GaAg*a*u | | | GUA | |
| 867 | g*c*AaAcA | 2443 | 908-928 | (VPmU)*A*cUg | 869 | 906-928 | UUGCAAACAG | 3584 |
| | gCcAuUcAc | | | GuGaAuGgCuGu | | | CCAUUCACCA | |
| | CaG*u*g | | | UuGc*a*a | | | GUG | |
| 868 | a*a*UgGcA | 2444 | 598-618 | (VPmU)*U*cUa | 870 | 596-618 | GCAAUGGCAG | 3585 |
| | gCcUcUgCu | | | AgCaGaGgCuGc | | | CCUCUGCUUA | |
| | UaG*a*a | | | CaUu*g*c | | | GAA | |
| 869 | c*a*AgAcG | 2445 | 1533-1553 | (VPmU)*U*cUg | 871 | 1531-1553 | GGCAAGACGU | 3586 |
| | uUcAaAuUu | | | AaAuUuGaAcGu | | | UCAAAUUUCA | |
| | CaG*a*g | | | CuUg*c*c | | | GAG | |
| 870 | u*c*GgAgA | 2446 | 2411-2431 | (VPmG)*C*gAu | 872 | 2409-2431 | CUUCGGAGAC | 3587 |
| | cUcCaGaCa | | | UgUcUgGaGuCu | | | UCCAGACAAU | |
| | AuC*g*c | | | CcGa*a*g | | | CGC | |
| 871 | c*c*CgGgC | 2447 | 2571-2591 | (VPmU)*U*uGa | 873 | 2569-2591 | GGCCCGGGCA | 3588 |
| | aGgCcCaGc | | | GcUgGgCcUgCc | | | GGCCCAGCUC | |
| | UcA*a*a | | | CgGg*c*c | | | AAA | |
| 872 | c*g*GaGaC | 2448 | 2412-2432 | (VPmG)*G*cGa | 874 | 2410-2432 | UUCGGAGACU | 3589 |
| | uCcAgAcAa | | | UuGuCuGgAgUc | | | CCAGACAAUC | |
| | UcG*c*c | | | UcCg*a*a | | | GCC | |
| 873 | c*g*AgCuG | 2449 | 2700-2720 | (VPmU)*A*gGc | 875 | 2698-2720 | UGCGAGCUGU | 3590 |
| | uGcAaCuAu | | | AuAgUuGcAcAg | | | GCAACUAUGC | |
| | GcC*u*g | | | CuCg*c*a | | | CUG | |
| 874 | g*a*CaGgG | 2450 | 1276-1296 | (VPmA)*U*uCa | 876 | 1274-1296 | UUGACAGGGU | 3591 |
| | uGcUgCgGu | | | AcCgCaGcAcCc | | | GCUGCGGUUG | |
| | UgA*a*u | | | UgUc*a*a | | | AAU | |
| 875 | g*g*UcAaG | 2451 | 1503-1523 | (VPmU)*C*gCa | 877 | 1501-1523 | CCGGUCAAGU | 3592 |
| | uCcAaGuCa | | | UgAcUuGgAcUu | | | CCAAGUCAUG | |
| | UgC*g*a | | | GaCc*g*g | | | CGA | |
| 876 | g*a*AuCcA | 2452 | 1293-1313 | (VPmG)*G*cUc | 878 | 1291-1313 | UUGAAUCCAA | 3593 |
| | aUgGcUaUg | | | CaUaGcCaUuGg | | | UGGCUAUGGA | |
| | GaG*c*c | | | AuUc*a*a | | | GCC | |
| 877 | u*g*CaAcU | 2453 | 2707-2727 | (VPmU)*U*gGg | 879 | 2705-2727 | UGUGCAACUA | 3594 |
| | aUgCcUgUg | | | CaCaGgCaUaGu | | | UGCCUGUGCC | |
| | CcC*a*g | | | UgCa*c*a | | | CAG | |
| 878 | a*g*GcCuU | 2454 | 2686-2706 | (VPmU)*A*gCu | 880 | 2684-2706 | AAAGGCCUUA | 3595 |
| | aUaAaUgCg | | | CgCaUuUaUaAg | | | UAAAUGCGAG | |
| | AgC*u*g | | | GcCu*u*u | | | CUG | |
| 879 | a*u*CaCcC | 2455 | 1259-1279 | (VPmU)*G*uCa | 881 | 1257-1279 | CCAUCACCCG | 3596 |
| | gAgUgCcUu | | | AaGgCaCuCgGg | | | AGUGCCUUUG | |
| | UgA*c*a | | | UgAu*g*g | | | ACA | |
| 880 | c*c*UgGcC | 2456 | 1248-1268 | (VPmU)*U*cGg | 882 | 1246-1268 | GCCCUGGCCA | 3597 |
| | aCcCaUcAc | | | GuGaUgGgUgGc | | | CCCAUCACCC | |
| | CcG*a*g | | | CaGg*g*c | | | GAG | |
| 881 | a*a*UuCaU | 2457 | 204-224 | (VPmA)*U*gGc | 883 | 202-224 | ACAAUUCAUC | 3598 |
| | cUuCcCuGc | | | GcAgGgAaGaUg | | | UUCCCUGCGC | |
| | GcC*a*u | | | AaUu*g*u | | | CAU | |
| 882 | c*c*CuGcG | 2458 | 214-234 | (VPmU)*A*aUa | 884 | 212-234 | UUCCCUGCGC | 3599 |
| | cCaUcUuUg | | | CaAaGaUgGcGc | | | CAUCUUUGUA | |
| | UaU*u*a | | | AgGg*a*a | | | UUA | |
| 883 | c*c*AcCcA | 2459 | 1253-1273 | (VPmA)*G*gCa | 885 | 1251-1273 | GGCCACCCAU | 3600 |
| | uCaCcCgAg | | | CuCgGgUgAuGg | | | CACCCGAGUG | |
| | UgC*c*u | | | GuGg*c*c | | | CCU | |
| 884 | u*g*GuUuC | 2460 | 931-951 | (VPmU)*G*cGu | 886 | 929-951 | CAUGGUUUCU | 3601 |
| | uCuUgCaAc | | | GuUgCaAgAgAa | | | CUUGCAACAC | |
| | AcG*c*a | | | AcCa*u*g | | | GCA | |
| 885 | g*g*CcUcC | 2461 | 2376-2396 | (VPmU)*C*uUu | 887 | 2374-2396 | GCGGCCUCCA | 3602 |
| | aGgCaGcUc | | | GaGcUgCcUgGa | | | GGCAGCUCAA | |
| | AaA*g*a | | | GgCc*g*c | | | AGA | |
| 886 | c*a*GcCaU | 2462 | 913-933 | (VPmU)*C*aUg | 888 | 911-933 | AACAGCCAUU | 3603 |
| | uCaCcAgUg | | | CaCuGgUgAaUg | | | CACCAGUGCA | |
| | CaU*g*g | | | GcUg*u*u | | | UGG | |
| 887 | g*u*CuCgC | 2463 | 387-407 | (VPmU)*U*gCc | 889 | 385-407 | AUGUCUCGCC | 3604 |
| | cGcAaGcAa | | | UuGcUuGcGgCg | | | GCAAGCAAGG | |
| | GgC*a*a | | | AgAc*a*u | | | CAA | |
| 888 | c*a*AaGgU | 2464 | 1402-1422 | (VPmG)*A*aUg | 890 | 1400-1422 | UGCAAAGGUU | 3605 |
| | uAcUgCaAc | | | GuUgCaGuAaCc | | | ACUGCAACCA | |
| | CaU*u*c | | | UuUg*c*a | | | UUC | |
| 889 | c*u*UuGaC | 2465 | 1272-1292 | (VPmA)*A*cCg | 891 | 1270-1292 | GCCUUUGACA | 3606 |
| | aGgGuGcUg | | | CaGcAcCcUgUc | | | GGGUGCUGCG | |
| | CgG*u*u | | | AaAg*g*c | | | GUU | |
| 890 | u*u*CaUcU | 2466 | 206-226 | (VPmA)*G*aUg | 892 | 204-226 | AAUUCAUCUU | 3607 |
| | uCcCuGcGc | | | GcGcAgGgAaGa | | | CCCUGCGCCA | |
| | CaU*c*u | | | UgAa*u*u | | | UCU | |
| 891 | g*g*UaUcC | 2467 | 1018-1038 | (VPmA)*C*cUa | 893 | 1016-1038 | UUGGUAUCCC | 3608 |
| | cUuCaGgAc | | | GuCcUgAaGgGa | | | UUCAGGACUA | |
| | UaG*g*u | | | UaCc*a*a | | | GGU | |
| 892 | g*c*UgUgC | 2468 | 2703-2723 | (VPmG)*C*aCa | 894 | 2701-2723 | GAGCUGUGCA | 3609 |
| | aAcUaUgCc | | | GgCaUaGuUgCa | | | ACUAUGCCUG | |
| | UgU*g*c | | | CaGc*u*c | | | UGC | |
| 893 | a*a*AgAuG | 2469 | 874-894 | (VPmG)*U*aGc | 895 | 872-894 | GUAAAGAUGA | 3610 |
| | aGcCcAgCa | | | UgCuGgGcUcAu | | | GCCCAGCAGC | |
| | GcU*a*c | | | CuUu*a*c | | | UAC | |
| 894 | g*a*GuGcC | 2470 | 1266-1286 | (VPmA)*G*cAc | 896 | 1264-1286 | CCGAGUGCCU | 3611 |
| | uUuGaCaGg | | | CcUgUcAaAgGc | | | UUGACAGGGU | |
| | GuG*c*u | | | AcUc*g*g | | | GCU | |
| 895 | a*g*AgGgC | 2471 | 2592-2612 | (VPmG)*U*gUc | 897 | 2590-2612 | AAAGAGGGCA | 3612 |
| | aGaCgCaGc | | | GcUgCgUcUgCc | | | GACGCAGCGA | |
| | GaC*a*c | | | CuCu*u*u | | | CAC | |
| 896 | a*u*CuUcC | 2472 | 209-229 | (VPmU)*A*aAg | 898 | 207-229 | UCAUCUUCCC | 3613 |
| | cUgCgCcAu | | | AuGgCgCaGgGa | | | UGCGCCAUCU | |
| | CuU*u*g | | | AgAu*g*a | | | UUG | |
| 897 | a*u*UcUuA | 2473 | 457-477 | (VPmU)*G*gUu | 899 | 455-477 | CCAUUCUUAC | 3614 |
| | cAgAuGaUg | | | CaUcAuCuGuAa | | | AGAUGAUGAA | |
| | AaC*c*a | | | GaAu*g*g | | | CCA | |
| 898 | c*c*UaUgC | 2474 | 1396-1416 | (VPmU)*U*gCa | 900 | 1394-1416 | GCCCUAUGCA | 3615 |
| | aAaGgUuAc | | | GuAaCcUuUgCa | | | AAGGUUACUG | |
| | UgC*a*a | | | UaGg*g*c | | | CAA | |
| 899 | g*u*CaAgU | 2475 | 1504-1524 | (VPmU)*U*cGc | 901 | 1502-1524 | CGGUCAAGUC | 3616 |
| | cCaAgUcAu | | | AuGaCuUgGaCu | | | CAAGUCAUGC | |
| | GcG*a*g | | | UgAc*c*g | | | GAG | |
| 900 | u*c*AcCcG | 2476 | 1260-1280 | (VPmU)*U*gUc | 902 | 1258-1280 | CAUCACCCGA | 3617 |
| | aGuGcCuUu | | | AaAgGcAcUcGg | | | GUGCCUUUGA | |
| | GaC*a*g | | | GuGa*u*g | | | CAG | |
| 901 | g*u*CcAcC | 2477 | 1184-1204 | (VPmA)*G*uGa | 903 | 1182-1204 | UAGUCCACCA | 3618 |
| | aCcGaGaCa | | | UgUcUcGgUgGu | | | CCGAGACAUC | |
| | UcA*c*u | | | GgAc*u*a | | | ACU | |
| 902 | c*g*UcAuC | 2478 | 728-748 | (VPmG)*G*cAa | 904 | 726-748 | AACGUCAUCU | 3619 |
| | uAgAgGaAu | | | AuUcCuCuAgAu | | | AGAGGAAUUU | |
| | UuG*c*c | | | GaCg*u*u | | | GCC | |
| 903 | g*u*GcAaC | 2479 | 2706-2726 | (VPmU)*G*gGc | 905 | 2704-2726 | CUGUGCAACU | 3620 |
| | uAuGcCuGu | | | AcAgGcAuAgUu | | | AUGCCUGUGC | |
| | GcC*c*a | | | GcAc*a*g | | | CCA | |
| 904 | g*g*CuCuC | 2480 | 193-213 | (VPmA)*A*gAu | 906 | 251-213 | ACGGCUCUCC | 3621 |
| | cCaCaAuUc | | | GaAuUgUgGgAg | | | CACAAUUCAU | |
| | AuC*u*u | | | AgCc*g*u | | | CUU | |
| 905 | u*a*AgCaA | 2481 | 419-439 | (VPmG)*C*gAg | 907 | 417-439 | CUUAAGCAAA | 3622 |
| | aCgGgAaUu | | | AaUuCcCgUuUg | | | CGGGAAUUCU | |
| | CuC*g*c | | | CuUa*a*g | | | CGC | |
| 906 | g*g*CcCuG | 2482 | 1245-1265 | (VPmG)*G*gUg | 908 | 1243-1265 | AUGGCCCUGG | 3623 |
| | gCcAcCcAu | | | AuGgGuGgCcAg | | | CCACCCAUCA | |
| | CaC*c*c | | | GgCc*a*u | | | CCC | |
| 907 | a*g*AaGcC | 2483 | 2668-2688 | (VPmU)*C*uUu | 909 | 2666-2688 | GGAGAAGCCA | 3624 |
| | aCaCgGgCg | | | CgCcCgUgUgGc | | | CACGGGCGAA | |
| | AaA*g*g | | | UuCu*c*c | | | AGG | |
| 908 | c*c*CgGcU | 2484 | 304-324 | (VPmG)*U*uCa | 910 | 302-324 | AACCCGGCUC | 3625 |
| | cUcCcGaUg | | | CaUcGgGaGaGc | | | UCCCGAUGUG | |
| | UgA*a*c | | | CgGg*u*u | | | AAC | |
| 909 | g*g*AgAaG | 2485 | 2666-2686 | (VPmU)*U*uCg | 911 | 2664-2686 | CAGGAGAAGC | 3626 |
| | cCaCaCgGg | | | CcCgUgUgGcUu | | | CACACGGGCG | |
| | CgA*a*a | | | CuCc*u*g | | | AAA | |
| 910 | u*g*CaAuG | 2486 | 595-615 | (VPmU)*A*aGc | 912 | 593-615 | AAUGCAAUGG | 3627 |
| | gCaGcCuCu | | | AgAgGcUgCcAu | | | CAGCCUCUGC | |
| | GcU*u*a | | | UgCa*u*u | | | UUA | |
| 911 | g*c*AcUcC | 2487 | 2451-2471 | (VPmU)*U*cCc | 913 | 2449-2471 | GAGCACUCCU | 3628 |
| | uCgGaGaAc | | | GuUcUcCgAgGa | | | CGGAGAACGG | |
| | GgG*a*g | | | GuGc*u*c | | | GAG | |
| 912 | u*u*GuUuA | 2488 | 717-737 | (VPmU)*U*aGa | 914 | 715-737 | GAUUGUUUAU | 3629 |
| | uCaAcGuCa | | | UgAcGuUgAuAa | | | CAACGUCAUC | |
| | UcU*a*g | | | AcAa*u*c | | | UAG | |
| 913 | c*a*GgGuG | 2489 | 1278-1298 | (VPmG)*G*aUu | 915 | 1276-1298 | GACAGGGUGC | 3630 |
| | cUgCgGuUg | | | CaAcCgCaGcAc | | | UGCGGUUGAA | |
| | AaU*c*c | | | CcUg*u*c | | | UCC | |
| 914 | u*a*UcGaG | 2490 | 573-593 | (VPmU)*G*uUu | 916 | 571-593 | UUUAUCGAGC | 3631 |
| | cAcAaAcGg | | | CcGuUuGuGcUc | | | ACAAACGGAA | |
| | AaA*c*a | | | GaUa*a*a | | | ACA | |
| 915 | c*u*CcCuC | 2491 | 1462-1482 | (VPmG)*G*cGg | 917 | 1460-1482 | CCCUCCCUCC | 3632 |
| | cUcUgCaAu | | | AuUgCaGaGgAg | | | UCUGCAAUCC | |
| | CcG*c*c | | | GgAg*g*g | | | GCC | |
| 916 | c*a*AgAaC | 2492 | 2637-2657 | (VPmG)*U*gAg | 918 | 2635-2657 | UUCAAGAACU | 3633 |
| | uGuAgCaAu | | | AuUgCuAcAgUu | | | GUAGCAAUCU | |
| | CuC*a*c | | | CuUg*a*a | | | CAC | |
| 917 | a*a*GuCcA | 2493 | 1507-1527 | (VPmG)*A*aCu | 919 | 1505-1527 | UCAAGUCCAA | 3634 |
| | aGuCaUgCg | | | CgCaUgAcUuGg | | | GUCAUGCGAG | |
| | AgU*u*c | | | AcUu*g*a | | | UUC | |
| 918 | g*u*AuCcC | 2494 | 1019-1039 | (VPmU)*A*cCu | 920 | 1017-1039 | UGGUAUCCCU | 3635 |
| | uUcAgGaCu | | | AgUcCuGaAgGg | | | UCAGGACUAG | |
| | AgG*u*g | | | AuAc*c*a | | | GUG | |
| 919 | u*c*CuUcU | 2495 | 291-311 | (VPmG)*A*gCc | 921 | 289-311 | UCUCCUUCUU | 3636 |
| | uUcUaAcCc | | | GgGuUaGaAaGa | | | UCUAACCCGG | |
| | GgC*u*c | | | AgGa*g*a | | | CUC | |
| 920 | c*c*AuGaC | 2496 | 186-206 | (VPmA)*U*uGu | 922 | 184-206 | AGCCAUGACG | 3637 |
| | gGcUcUcCc | | | GgGaGaGcCgUc | | | GCUCUCCCAC | |
| | AcA*a*u | | | AuGg*c*u | | | AAU | |
| 921 | u*u*UgAcA | 2497 | 1273-1293 | (VPmU)*A*aCc | 923 | 1271-1293 | CCUUUGACAG | 3638 |
| | gGgUgCuGc | | | GcAgCaCcCuGu | | | GGUGCUGCGG | |
| | GgU*u*g | | | CaAa*g*g | | | UUG | |
| 922 | g*c*GgGuU | 2498 | 1011-1031 | (VPmU)*C*uGa | 924 | 1009-1031 | CCGCGGGUUG | 3639 |
| | gGuAuCcCu | | | AgGgAuAcCaAc | | | GUAUCCCUUC | |
| | UcA*g*g | | | CcGc*g*g | | | AGG | |
| 923 | u*g*UgCaA | 2499 | 2705-2725 | (VPmG)*G*gCa | 925 | 2703-2725 | GCUGUGCAAC | 3640 |
| | cUaUgCcUg | | | CaGgCaUaGuUg | | | UAUGCCUGUG | |
| | UgC*c*c | | | CaCa*g*c | | | CCC | |
| 924 | g*u*GcUgC | 2500 | 1282-1302 | (VPmU)*A*uUg | 926 | 1280-1302 | GGGUGCUGCG | 3641 |
| | gGuUgAaUc | | | GaUuCaAcCgCa | | | GUUGAAUCCA | |
| | CaA*u*g | | | GcAc*c*c | | | AUG | |
| 925 | c*a*GcAcU | 2501 | 412-432 | (VPmU)*U*cCc | 927 | 410-432 | CCCAGCACUU | 3642 |
| | uAaGcAaAc | | | GuUuGcUuAaGu | | | AAGCAAACGG | |
| | GgG*a*a | | | GcUg*g*g | | | GAA | |
| 926 | u*u*GaAuC | 2502 | 1291-1311 | (VPmU)*U*cCa | 928 | 1289-1311 | GGUUGAAUCC | 3643 |
| | cAaUgGcUa | | | UaGcCaUuGgAu | | | AAUGGCUAUG | |
| | UgG*a*g | | | UcAa*c*c | | | GAG | |
| 927 | a*a*GaAcU | 2503 | 2638-2658 | (VPmA)*G*uGa | 929 | 2636-2658 | UCAAGAACUG | 3644 |
| | gUaGcAaUc | | | GaUuGcUaCaGu | | | UAGCAAUCUC | |
| | UcA*c*u | | | UcUu*g*a | | | ACU | |
| 928 | a*u*UcAcC | 2504 | 918-938 | (VPmA)*G*aAa | 930 | 916-938 | CCAUUCACCA | 3645 |
| | aGuGcAuGg | | | CcAuGcAcUgGu | | | GUGCAUGGUU | |
| | UuU*c*u | | | GaAu*g*g | | | UCU | |
| 929 | c*u*GgCaG | 2505 | 1144-1164 | (VPmU)*G*gAa | 931 | 1142-1164 | GCCUGGCAGA | 3646 |
| | aAgGgCgCu | | | AgCgCcCuUcUg | | | AGGGCGCUUU | |
| | UuC*c*a | | | CcAg*g*c | | | CCA | |
| 930 | g*g*AuGaG | 2506 | 839-859 | (VPmG)*G*gCa | 932 | 837-859 | UGGGAUGAGU | 3647 |
| | uGcAgAaUa | | | UaUuCuGcAcUc | | | GCAGAAUAUG | |
| | UgC*c*c | | | AuCc*c*a | | | CCC | |
| 931 | u*c*UcCuU | 2507 | 289-309 | (VPmG)*C*cGg | 933 | 287-309 | AGUCUCCUUC | 3648 |
| | CUuUcUaAc | | | GuUaGaAaGaAg | | | UUUCUAACCC | |
| | CcG*g*c | | | GaGa*c*u | | | GGC | |
| 932 | u*g*CuGcG | 2508 | 1283-1303 | (VPmU)*C*aUu | 934 | 1281-1303 | GGUGCUGCGG | 3649 |
| | gUuGaAuCc | | | GgAuUcAaCcGc | | | UUGAAUCCAA | |
| | AaU*g*g | | | AgCa*c*c | | | UGG | |
| 933 | g*g*AgUCU | 2509 | 285-305 | (VPmG)*G*uUa | 935 | 283-305 | CUGGAGUCUC | 3650 |
| | cCuUcUuUc | | | GaAaGaAgGaGa | | | CUUCUUUCUA | |
| | UaA*c*c | | | CuCc*a*g | | | ACC | |
| 934 | g*c*CcUgG | 2510 | 1246-1266 | (VPmU)*G*gGu | 936 | 1244-1266 | UGGCCCUGGC | 3651 |
| | cCaCcCaUc | | | GaUgGgUgGcCa | | | CACCCAUCAC | |
| | Acc*c*g | | | GgGc*c*a | | | CCG | |
| 935 | c*g*GcUcU | 2511 | 192-212 | (VPmA)*G*aUg | 937 | 190-212 | GACGGCUCUC | 3652 |
| | cCcAcAaUu | | | AaUuGuGgGaGa | | | CCACAAUUCA | |
| | CaU*c*u | | | GcCg*u*c | | | UCU | |
| 936 | g*a*GaAgC | 2512 | 2667-2687 | (VPmU)*U*uUc | 938 | 2665-2687 | AGGAGAAGCC | 3653 |
| | cAcAcGgGc | | | GcCcGuGuGgCu | | | ACACGGGCGA | |
| | GaA*a*g | | | UcUc*c*u | | | AAG | |
| 937 | g*u*UuAgU | 2513 | 1179-1199 | (VPmU)*G*uCu | 939 | 1177-1199 | CUGUUUAGUC | 3654 |
| | cCaCcAcCg | | | CgGuGgUgGaCu | | | CACCACCGAG | |
| | AgA*c*a | | | AaAc*a*g | | | ACA | |
| 938 | u*g*GcCcU | 2514 | 1244-1264 | (VPmG)*G*uGa | 940 | 1242-1264 | GAUGGCCCUG | 3655 |
| | gGcCaCcCa | | | UgGgUgGcCaGg | | | GCCACCCAUC | |
| | UcA*c*c | | | GcCa*u*c | | | ACC | |
| 939 | a*u*AaAcU | 2515 | 770-790 | (VPmU)*C*cUc | 941 | 768-790 | AGAUAAACUU | 3656 |
| | uCuGcAcUg | | | CaGuGcAgAaGu | | | CUGCACUGGA | |
| | GaG*g*g | | | UuAu*c*u | | | GGG | |
| 940 | c*u*AaCcC | 2516 | 300-320 | (VPmA)*C*aUc | 942 | 298-320 | UUCUAACCCG | 3657 |
| | gGcUcUcCc | | | GgGaGaGcCgGg | | | GCUCUCCCGA | |
| | GaU*g*u | | | UuAg*a*a | | | UGU | |
| 941 | g*a*AcAcU | 2517 | 954-974 | (VPmA)*U*uCu | 943 | 952-974 | CAGAACACUC | 3658 |
| | cAuGgAuUa | | | UaAuCcAuGaGu | | | AUGGAUUAAG | |
| | AgA*a*u | | | GuUc*u*g | | | AAU | |
| 942 | u*u*AuAaA | 2518 | 2691-2711 | (VPmU)*U*gCa | 944 | 2689-2711 | CCUUAUAAAU | 3659 |
| | uGcGaGcUg | | | CaGcUcGcAuUu | | | GCGAGCUGUG | |
| | UgC*a*a | | | AuAa*g*g | | | CAA | |
| 943 | a*u*UuCuC | 2519 | 1325-1345 | (VPmU)*U*cUa | 945 | 1323-1345 | GGAUUUCUCU | 3660 |
| | uAgGaGaCu | | | AgUcUcCuAgAg | | | AGGAGACUUA | |
| | UaG*a*g | | | AaAu*c*c | | | GAG | |
| 944 | u*u*CuUuC | 2520 | 294-314 | (VPmG)*G*aGa | 946 | 292-314 | CCUUCUUUCU | 3661 |
| | uAaCcCgGc | | | GcCgGgUuAgAa | | | AACCCGGCUC | |
| | UcU*c*c | | | AgAa*g*g | | | UCC | |
| 945 | u*u*GaCaG | 2521 | 1274-1294 | (VPmU)*C*aAc | 947 | 1272-1294 | CUUUGACAGG | 3662 |
| | gGuGcUgCg | | | CgCaGcAcCcUg | | | GUGCUGCGGU | |
| | GuU*g*a | | | UcAa*a*g | | | UGA | |
| 946 | a*g*CaCuC | 2522 | 2450-2470 | (VPmU)*C*cCg | 948 | 2448-2470 | GGAGCACUCC | 3663 |
| | cUcGgAgAa | | | UuCuCcGaGgAg | | | UCGGAGAACG | |
| | CgG*g*a | | | UgCu*c*c | | | GGA | |
| 947 | u*c*UcUuG | 2523 | 936-956 | (VPmU)*U*cUg | 949 | 934-956 | UUUCUCUUGC | 3664 |
| | cAaCaCgCa | | | UgCgUgUuGcAa | | | AACACGCACA | |
| | CaG*a*a | | | GaGa*a*a | | | GAA | |
| 948 | c*c*CuGgC | 2524 | 1247-1267 | (VPmU)*C*gGg | 950 | 1245-1267 | GGCCCUGGCC | 3665 |
| | CAcCcAuCa | | | UgAuGgGuGgCc | | | ACCCAUCACC | |
| | CcC*g*a | | | AgGg*c*c | | | CGA | |
| 949 | a*g*CaAaC | 2525 | 421-441 | (VPmG)*G*gCg | 951 | 419-441 | UAAGCAAACG | 3666 |
| | gGgAaUuCu | | | AgAaUuCcCgUu | | | GGAAUUCUCG | |
| | CgC*c*c | | | UgCu*u*a | | | CCC | |
| 950 | g*c*UgGcA | 2526 | 1347-1367 | (VPmU)*U*aGa | 952 | 1345-1367 | GAGCUGGCAG | 3667 |
| | gGgAaCaCg | | | CgUgUuCcCuGc | | | GGAACACGUC | |
| | UcU*a*g | | | CaGc*u*c | | | UAG | |
| 951 | g*a*GaCuC | 2527 | 2414-2434 | (VPmA)*A*gGc | 953 | 2412-2434 | CGGAGACUCC | 3668 |
| | cAgAcAaUc | | | GaUuGuCuGgAg | | | AGACAAUCGC | |
| | GcC*u*u | | | UcUc*c*g | | | CUU | |
| 952 | c*c*CuAuG | 2528 | 1395-1415 | (VPmU)*G*cAg | 954 | 1393-1415 | AGCCCUAUGC | 3669 |
| | cAaAgGuUa | | | UaAcCuUuGcAu | | | AAAGGUUACU | |
| | CuG*c*a | | | AgGg*c*u | | | GCA | |
| 953 | a*a*GaAuA | 2529 | 1107-1127 | (VPmG)*A*uAc | 955 | 1105-1127 | CUAAGAAUAC | 3670 |
| | cCaGgAuCa | | | UgAuCcUgGuAu | | | CAGGAUCAGU | |
| | GuA*u*c | | | UcUu*a*g | | | AUC | |
| 954 | u*c*AuCuU | 2530 | 207-227 | (VPmA)*A*gAu | 956 | 205-227 | AUUCAUCUUC | 3671 |
| | cCcUgCgCc | | | GgCgCaGgGaAg | | | CCUGCGCCAU | |
| | AuC*u*u | | | AuGa*a*u | | | CUU | |
| 955 | c*u*UcUuU | 2531 | 293-313 | (VPmG)*A*gAg | 957 | 291-313 | UCCUUCUUUC | 3672 |
| | cUaAcCcGg | | | CcGgGuUaGaAa | | | UAACCCGGCU | |
| | CuC*u*c | | | GaAg*g*a | | | CUC | |
| 956 | c*c*AcCaC | 2532 | 1186-1206 | (VPmU)*A*aGu | 958 | 1184-1206 | GUCCACCACC | 3673 |
| | cGaGaCaUc | | | GaUgUcUcGgUg | | | GAGACAUCAC | |
| | AcU*u*g | | | GuGg*a*c | | | UUG | |
| 957 | a*a*CcAuU | 2533 | 1415-1435 | (VPmU)*G*cUa | 959 | 1413-1435 | GCAACCAUUC | 3674 |
| | cCaGcCaGg | | | CcUgGcUgGaAu | | | CAGCCAGGUA | |
| | UaG*c*a | | | GgUu*g*c | | | GCA | |
| 958 | u*c*UuCcC | 2534 | 210-230 | (VPmA)*C*aAa | 960 | 208-230 | CAUCUUCCCU | 3675 |
| | uGcGcCaUc | | | GaUgGcGcAgGg | | | GCGCCAUCUU | |
| | UuU*g*u | | | AaGa*u*g | | | UGU | |
| 959 | c*u*GgCcA | 2535 | 1249-1269 | (VPmA)*C*uCg | 961 | 1247-1269 | CCCUGGCCAC | 3676 |
| | cCcAuCaCc | | | GgUgAuGgGuGg | | | CCAUCACCCG | |
| | CgA*g*u | | | CcAg*g*g | | | AGU | |
| 960 | g*a*GgGcA | 2536 | 2593-2613 | (VPmA)*G*uGu | 962 | 2591-2613 | AAGAGGGCAG | 3677 |
| | gAcGcAgCg | | | CgCuGcGuCuGc | | | ACGCAGCGAC | |
| | AcA*c*u | | | CcUc*u*u | | | ACU | |
| 961 | c*a*CaAuU | 2537 | 201-221 | (VPmG)*C*gCa | 963 | 199-221 | CCCACAAUUC | 3678 |
| | cAuCuUcCc | | | GgGaAgAuGaAu | | | AUCUUCCCUG | |
| | UgC*g*c | | | UgUg*g*g | | | CGC | |
| 962 | c*c*AuCaC | 2538 | 1257-1277 | (VPmU)*C*aAa | 964 | 1255-1277 | ACCCAUCACC | 3679 |
| | cCgAgUgCc | | | GgCaCuCgGgUg | | | CGAGUGCCUU | |
| | UuU*g*a | | | AuGg*g*u | | | UGA | |
| 963 | a*c*AcGcA | 2539 | 945-965 | (VPmU)*C*aUg | 965 | 943-965 | CAACACGCAC | 3680 |
| | cAgAaCaCu | | | AgUgUuCuGuGc | | | AGAACACUCA | |
| | CaU*g*g | | | GuGu*u*g | | | UGG | |
| 964 | g*a*AgCcA | 2540 | 2669-2689 | (VPmG)*C*cUu | 966 | 2667-2689 | GAGAAGCCAC | 3681 |
| | cAcGgGcGa | | | UcGcCcGuGuGg | | | ACGGGCGAAA | |
| | AaG*g*c | | | CuUc*u*c | | | GGC | |
| 965 | c*a*UcACC | 2541 | 1258-1278 | (VPmG)*U*cAa | 967 | 1256-1278 | CCCAUCACCC | 3682 |
| | cGaGuGcCu | | | AgGcAcUcGgGu | | | GAGUGCCUUU | |
| | UuG*a*c | | | GaUg*g*g | | | GAC | |
| 966 | u*c*AaGuU | 2542 | 28-48 | (VPmG)*U*gAc | 968 | 26-48 | GUUCAAGUUC | 3683 |
| | cGcAgGgAc | | | GuCcCuGcGaAc | | | GCAGGGACGU | |
| | GuC*a*c | | | UuGa*a*c | | | CAC | |
| 967 | u*c*UaAcC | 2543 | 299-319 | (VPmU)*A*uCg | 969 | 297-319 | UUUCUAACCC | 3684 |
| | cGgCuCuCc | | | GgAgAgCcGgGu | | | GGCUCUCCCG | |
| | CgA*u*g | | | UaGa*a*a | | | AUG | |
| 968 | g*g*GaAgU | 2544 | 2532-2552 | (VPmG)*G*cGu | 970 | 2530-2552 | ACGGGAAGUG | 3685 |
| | gGaGgGaGc | | | GcUcCcUcCaCu | | | GAGGGAGCAC | |
| | AcG*c*c | | | UcCc*g*u | | | GCC | |
| 969 | c*c*UgGcA | 2545 | 1143-1163 | (VPmG)*G*aAa | 971 | 1141-1163 | GGCCUGGCAG | 3686 |
| | gAaGgGcGc | | | GcGcCcUuCuGc | | | AAGGGCGCUU | |
| | UuU*c*c | | | CaGg*c*c | | | UCC | |
| 970 | c*u*CgCcG | 2546 | 389-409 | (VPmG)*U*uUg | 972 | 387-409 | GUCUCGCCGC | 3687 |
| | cAaGcAaGg | | | CcUuGcUuGcGg | | | AAGCAAGGCA | |
| | CaA*a*c | | | CgAg*a*c | | | AAC | |
| 971 | g*a*GcUgG | 2547 | 1345-1365 | (VPmA)*G*aCg | 973 | 1343-1365 | GAGAGCUGGC | 3688 |
| | cAgGgAaCa | | | UgUuCcCuGcCa | | | AGGGAACACG | |
| | CgU*c*u | | | GcUc*u*c | | | UCU | |
| 972 | a*c*CaGuG | 2548 | 922-942 | (VPmU)*A*aGa | 974 | 920-942 | UCACCAGUGC | 3689 |
| | cAuGgUuUc | | | GaAaCcAuGcAc | | | AUGGUUUCUC | |
| | UcU*u*g | | | UgGu*g*a | | | UUG | |
| 973 | g*c*AuCaA | 2549 | 2288-2308 | (VPmA)*C*uCc | 975 | 2286-2308 | GCGCAUCAAG | 3690 |
| | gCuCgAgAa | | | UuCuCgAgCuUg | | | CUCGAGAAGG | |
| | GgA*g*u | | | AuGc*g*c | | | AGU | |
| 974 | c*u*UcCuU | 2550 | 2400-2420 | (VPmG)*A*gUc | 976 | 2398-2420 | CCCUUCCUUA | 3691 |
| | aGcUuCgGa | | | UcCgAaGcUaAg | | | GCUUCGGAGA | |
| | GaC*u*c | | | GaAg*g*g | | | CUC | |
| 975 | g*a*GgAuG | 2551 | 706-726 | (VPmU)*G*aUa | 977 | 704-726 | CAGAGGAUGA | 3692 |
| | aCgAuUgUu | | | AaCaAuCgUcAu | | | CGAUUGUUUA | |
| | Uau*c*a | | | CcUc*u*g | | | UCA | |
| 976 | c*a*UcCaG | 2552 | 10-30 | (VPmU)*G*aAc | 978 | 8-30 | UCCAUCCAGA | 3693 |
| | aCuCcUgAc | | | GuCaGgAgUcUg | | | CUCCUGACGU | |
| | GuU*c*a | | | GaUg*g*a | | | UCA | |
| 977 | c*a*GgGaC | 2553 | 2127-2147 | (VPmU)*C*uUc | 979 | 2125-2147 | CACAGGGACA | 3694 |
| | aCuUgCgAc | | | GuCgCaAgUgUc | | | CUUGCGACGA | |
| | GaA*g*a | | | CcUg*u*g | | | AGA | |
| 978 | a*c*CaAaG | 2554 | 5939-5959 | (VPmU)*A*gGu | 980 | 5937-5959 | UGACCAAAGG | 3695 |
| | gUcAuUaCa | | | UgUaAuGaCcUu | | | UCAUUACAAC | |
| | AcC*u*g | | | UgGu*c*a | | | CUG | |
| 979 | c*g*UuCaA | 2555 | 1538-1558 | (VPmG)*G*uUg | 981 | 1536-1558 | GACGUUCAAA | 3696 |
| | aUuUcAgAg | | | CuCuGaAaUuUg | | | UUUCAGAGCA | |
| | CaA*c*c | | | AaCg*u*c | | | ACC | |
| 980 | c*u*CcAuG | 2556 | 1063-1083 | (VPmU)*G*cAa | 982 | 1061-1083 | CUCUCCAUGG | 3697 |
| | gGaUuCaUa | | | UaUgAaUcCcAu | | | GAUUCAUAUU | |
| | UuG*c*a | | | GgAg*a*g | | | GCA | |
| 981 | c*a*GcCcU | 2557 | 1392-1412 | (VPmA)*G*uAa | 983 | 1390-1412 | CCCAGCCCUA | 3698 |
| | aUgCaAaGg | | | CcUuUgCaUaGg | | | UGCAAAGGUU | |
| | UuA*c*u | | | GcUg*g*g | | | ACU | |
| 982 | c*a*CaGgG | 2558 | 2125-2145 | (VPmU)*U*cGu | 984 | 2123-2145 | GCCACAGGGA | 3699 |
| | aCaCuUgCg | | | CgCaAgUgUcCc | | | CACUUGCGAC | |
| | AcG*a*a | | | UgUg*g*c | | | GAA | |
| 983 | a*u*AcCaG | 2559 | 1111-1131 | (VPmU)*C*uCg | 985 | 1109-1131 | GAAUACCAGG | 3700 |
| | gAuCaGuAu | | | AuAcUgAuCcUg | | | AUCAGUAUCG | |
| | CgA*g*a | | | GuAu*u*c | | | AGA | |
| 984 | g*c*AuCcA | 2560 | 667-687 | (VPmA)*A*cCu | 986 | 665-687 | AAGCAUCCAA | 3701 |
| | aUcCcGuGg | | | CcAcGgGaUuGg | | | UCCCGUGGAG | |
| | AgG*u*u | | | AuGc*u*u | | | GUU | |
| 985 | g*c*CcAaC | 2561 | 2331-2351 | (VPmU)*A*cAc | 987 | 2329-2351 | AUGCCCAACA | 3702 |
| | aCgGaGaAc | | | GuUcUcCgUgUu | | | CGGAGAACGU | |
| | GuG*u*a | | | GgGc*a*u | | | GUA | |
| 986 | c*c*AgCcA | 2562 | 1053-1073 | (VPmA)*U*cCc | 988 | 1051-1073 | UCCCAGCCAC | 3703 |
| | cCuCuCcAu | | | AuGgAgAgGuGg | | | CUCUCCAUGG | |
| | GgG*a*u | | | CuGg*g*a | | | GAU | |
| 987 | a*g*UgAuC | 2563 | 2854-2874 | (VPmA)*U*uAu | 989 | 2852-2874 | ACAGUGAUCG | 3704 |
| | gAgUgUuGa | | | UcAaCaCuCgAu | | | AGUGUUGAAU | |
| | AuA*a*u | | | CaCu*g*u | | | AAU | |
| 988 | c*c*AcCuC | 2564 | 1057-1077 | (VPmA)*U*gAa | 990 | 1055-1077 | AGCCACCUCU | 3705 |
| | uCcAuGgGa | | | UcCcAuGgAgAg | | | CCAUGGGAUU | |
| | UuC*a*u | | | GuGg*c*u | | | CAU | |
| 989 | g*a*AuAcC | 2565 | 1109-1129 | (VPmU)*C*gAu | 991 | 1107-1129 | AAGAAUACCA | 3706 |
| | aGgAuCaGu | | | AcUgAuCcUgGu | | | GGAUCAGUAU | |
| | AuC*g*a | | | AuUc*u*u | | | CGA | |
| 990 | c*a*CaGuG | 2566 | 2851-2871 | (VPmA)*U*uCa | 992 | 2849-2871 | GGCACAGUGA | 3707 |
| | aUcGaGuGu | | | AcAcUcGaUcAc | | | UCGAGUGUUG | |
| | UgA*a*u | | | UgUg*c*c | | | AAU | |
| 991 | c*g*GcCcA | 2567 | 1387-1407 | (VPmU)*C*uUu | 993 | 1385-1407 | GCCGGCCCAG | 3708 |
| | gCcCuAuGc | | | GcAuAgGgCuGg | | | CCCUAUGCAA | |
| | AaA*g*g | | | GcCg*g*c | | | AGG | |
| 992 | g*g*CaCaG | 2568 | 2849-2869 | (VPmU)*C*aAc | 994 | 2847-2869 | AUGGCACAGU | 3709 |
| | uGaUcGaGu | | | AcUcGaUcAcUg | | | GAUCGAGUGU | |
| | GuU*g*a | | | UgCc*a*u | | | UGA | |
| 993 | c*c*UgGgC | 2569 | 2079-2099 | (VPmU)*G*cUu | 995 | 2077-2099 | GUCCUGGGCG | 3710 |
| | gAgAaGcAu | | | AuGcUuCuCgCc | | | AGAAGCAUAA | |
| | AaG*c*g | | | CaGg*a*c | | | GCG | |
| 994 | u*c*CuGaC | 2570 | 19-39 | (VPmU)*U*gCg | 996 | 17-39 | ACUCCUGACG | 3711 |
| | gUuCaAgUu | | | AaCuUgAaCgUc | | | UUCAAGUUCG | |
| | CgC*a*g | | | AgGa*g*u | | | CAG | |
| 995 | g*a*UcGaG | 2571 | 2857-2877 | (VPmA)*U*cAu | 997 | 2855-2877 | GUGAUCGAGU | 3712 |
| | uGuUgAaUa | | | UaUuCaAcAcUc | | | GUUGAAUAAU | |
| | AuG*a*u | | | GaUc*a*c | | | GAU | |
| 996 | a*c*GcCaG | 2572 | 700-720 | (VPmA)*C*aAu | 998 | 698-720 | UCACGCCAGA | 3713 |
| | aGgAuGaCg | | | CgUcAuCcUcUg | | | GGAUGACGAU | |
| | AuU*g*u | | | GcGu*g*a | | | UGU | |
| 997 | a*u*UcAuA | 2573 | 1072-1092 | (VPmG)*U*uAu | 999 | 1070-1092 | GGAUUCAUAU | 3714 |
| | uUgCaGaCa | | | UgUcUgCaAuAu | | | UGCAGACAAU | |
| | AuA*a*c | | | GaAu*c*c | | | AAC | |
| 998 | g*g*UuGgC | 2574 | 684-704 | (VPmG)*G*cGu | 1000 | 682-704 | GAGGUUGGCA | 3715 |
| | aUcCaGgUc | | | GaCcUgGaUgCc | | | UCCAGGUCAC | |
| | AcG*c*c | | | AaCc*u*c | | | GCC | |
| 999 | g*u*GgCcA | 2575 | 1783-1803 | (VPmU)*U*cGc | 1001 | 1781-1803 | UGGUGGCCAA | 3716 |
| | aGuUcAaGa | | | UcUuGaAcUuGg | | | GUUCAAGAGC | |
| | GcG*a*g | | | CcAc*c*a | | | GAG | |
| 1000 | a*u*GgCaC | 2576 | 2847-2867 | (VPmA)*A*cAc | 1002 | 2845-2867 | AAAUGGCACA | 3717 |
| | aGuGaUcGa | | | UcGaUcAcUgUg | | | GUGAUCGAGU | |
| | GuG*u*u | | | CcAu*u*u | | | GUU | |
| 1001 | u*a*CaAgU | 2577 | 1597-1617 | (VPmG)*U*cGc | 1003 | 1595-1617 | CCUACAAGUG | 3718 |
| | gCaAcCuGu | | | AcAgGuUgCaCu | | | CAACCUGUGC | |
| | GcG*a*c | | | UgUa*g*g | | | GAC | |
| 1002 | a*g*AuCcC | 2578 | 2394-2414 | (VPmU)*C*gAa | 1004 | 2392-2414 | AAAGAUCCCU | 3719 |
| | uUcCuUaGc | | | GcUaAgGaAgGg | | | UCCUUAGCUU | |
| | UuC*g*g | | | AuCu*u*u | | | CGG | |
| 1003 | c*a*UcAaG | 2579 | 2289-2309 | (VPmA)*A*cUc | 1005 | 2287-2309 | CGCAUCAAGC | 3720 |
| | CUcGaGaAg | | | CuUcUcGaGcUu | | | UCGAGAAGGA | |
| | GaG*u*u | | | GaUg*c*g | | | GUU | |
| 1004 | u*c*CaGaC | 2580 | 12-32 | (VPmU)*U*uGa | 1006 | 10-32 | CAUCCAGACU | 3721 |
| | uCcUgAcGu | | | AcGuCaGgAgUc | | | CCUGACGUUC | |
| | UcA*a*g | | | UgGa*u*g | | | AAG | |
| 1005 | g*a*GcUgG | 2581 | 2497-2517 | (VPmU)*G*aGa | 1007 | 2495-2517 | GGGAGCUGGA | 3722 |
| | aCgGaGgGa | | | UcCcUcCgUcCa | | | CGGAGGGAUC | |
| | UcU*c*g | | | GcUc*c*c | | | UCG | |
| 1006 | a*c*AgGgA | 2582 | 2126-2146 | (VPmU)*U*uCg | 1008 | 2124-2146 | CCACAGGGAC | 3723 |
| | cAcUuGcGa | | | UcGcAaGuGuCc | | | ACUUGCGACG | |
| | CgA*a*g | | | CuGu*g*g | | | AAG | |
| 1007 | c*c*UuCcU | 2583 | 2399-2419 | (VPmA)*G*uCu | 1009 | 2397-2419 | UCCCUUCCUU | 3724 |
| | uAgCuUcGg | | | CcGaAgCuAaGg | | | AGCUUCGGAG | |
| | AgA*c*u | | | AaGg*g*a | | | ACU | |
| 1008 | a*g*GuCcU | 2584 | 2075-2095 | (VPmU)*A*uGc | 1010 | 2073-2095 | CCAGGUCCUG | 3725 |
| | gGgCgAgAa | | | UuCuCgCcCaGg | | | GGCGAGAAGC | |
| | GcA*u*a | | | AcCu*g*g | | | AUA | |
| 1009 | a*u*UuCaG | 2585 | 1545-1565 | (VPmA)*C*cAc | 1011 | 1543-1565 | AAAUUUCAGA | 3726 |
| | aGcAaCcUg | | | CaGgUuGcUcUg | | | GCAACCUGGU | |
| | GuG*g*u | | | AaAu*u*u | | | GGU | |
| 1010 | a*g*AgGaU | 2586 | 705-725 | (VPmG)*A*uAa | 1012 | 703-725 | CCAGAGGAUG | 3727 |
| | gAcGaUuGu | | | AcAaUcGuCaUc | | | ACGAUUGUUU | |
| | UuA*u*c | | | CuCu*g*g | | | AUC | |
| 1011 | u*g*GuGgC | 2587 | 1781-1801 | (VPmU)*G*cUc | 1013 | 1779-1801 | CGUGGUGGCC | 3728 |
| | cAaGuUcAa | | | UuGaAcUuGgCc | | | AAGUUCAAGA | |
| | GaG*c*g | | | AcCa*c*g | | | GCG | |
| 1012 | g*c*CgGcC | 2588 | 1385-1405 | (VPmU)*U*uGc | 1014 | 1383-1405 | AGGCCGGCCC | 3729 |
| | cAgCcCuAu | | | AuAgGgCuGgGc | | | AGCCCUAUGC | |
| | GcA*a*a | | | CgGc*c*u | | | AAA | |
| 1013 | u*c*AuAuU | 2589 | 1074-1094 | (VPmG)*G*gUu | 1015 | 1072-1094 | AUUCAUAUUG | 3730 |
| | gCaGaCaAu | | | AuUgUcUgCaAu | | | CAGACAAUAA | |
| | AaC*c*c | | | AuGa*a*u | | | CCC | |
| 1014 | a*g*CcAcC | 2590 | 1055-1075 | (VPmG)*A*aUc | 1016 | 1053-1075 | CCAGCCACCU | 3731 |
| | uCuCcAuGg | | | CcAuGgAgAgGu | | | CUCCAUGGGA | |
| | GaU*u*c | | | GgCu*g*g | | | UUC | |
| 1015 | u*c*UaAgC | 2591 | 2281-2301 | (VPmU)*U*cGa | 1017 | 2279-2301 | UCUCUAAGCG | 3732 |
| | gCaUcAaGc | | | GcUuGaUgCgCu | | | CAUCAAGCUC | |
| | UcG*a*g | | | UaGa*g*a | | | GAG | |
| 1016 | a*g*GgAaC | 2592 | 1353-1373 | (VPmG)*G*uGg | 1018 | 1351-1373 | GCAGGGAACA | 3733 |
| | aCgUcUaGc | | | GcUaGaCgUgUu | | | CGUCUAGCCC | |
| | CcA*c*c | | | CcCu*g*c | | | ACC | |
| 1017 | c*c*UcUcC | 2593 | 1060-1080 | (VPmA)*A*uAu | 1019 | 1058-1080 | CACCUCUCCA | 3734 |
| | aUgGgAuUc | | | GaAuCcCaUgGa | | | UGGGAUUCAU | |
| | AuA*u*u | | | GaGg*u*g | | | AUU | |
| 1018 | c*a*CcUcU | 2594 | 1058-1078 | (VPmU)*A*uGa | 1020 | 1056-1078 | GCCACCUCUC | 3735 |
| | cCaUgGgAu | | | AuCcCaUgGaGa | | | CAUGGGAUUC | |
| | UcA*u*a | | | GgUg*g*c | | | AUA | |
| 1019 | g*g*AuUcA | 2595 | 1070-1090 | (VPmU)*A*uUg | 1021 | 1068-1090 | UGGGAUUCAU | 3736 |
| | uAuUgCaGa | | | UcUgCaAuAuGa | | | AUUGCAGACA | |
| | CaA*u*a | | | AuCc*c*a | | | AUA | |
| 1020 | u*g*UgGgC | 2596 | 526-546 | (VPmG)*U*uCa | 1022 | 524-546 | CCUGUGGGCA | 3737 |
| | aGuGcCaGa | | | UcUgGcAcUgCc | | | GUGCCAGAUG | |
| | UgA*a*c | | | CaCa*g*g | | | AAC | |
| 1021 | g*c*CaCcU | 2597 | 1056-1076 | (VPmU)*G*aAu | 1023 | 1054-1076 | CAGCCACCUC | 3738 |
| | cUcCaUgGg | | | CcCaUgGaGaGg | | | UCCAUGGGAU | |
| | AuU*c*a | | | UgGc*u*g | | | UCA | |
| 1022 | c*c*AaCaC | 2598 | 2333-2353 | (VPmA)*G*uAc | 1024 | 2331-2353 | GCCCAACACG | 3739 |
| | gGaGaAcGu | | | AcGuUcUcCgUg | | | GAGAACGUGU | |
| | GuA*c*u | | | UuGg*g*c | | | ACU | |
| 1023 | a*a*UaCcA | 2599 | 1110-1130 | (VPmU)*U*cGa | 1025 | 1108-1130 | AGAAUACCAG | 3740 |
| | gGaUcAgUa | | | UaCuGaUcCuGg | | | GAUCAGUAUC | |
| | UcG*a*g | | | UaUu*c*u | | | GAG | |
| 1024 | c*c*UcCuC | 2600 | 516-536 | (VPmU)*A*cUg | 1026 | 514-536 | GACCUCCUCA | 3741 |
| | aCcUgUgGg | | | CcCaCaGgUgAg | | | CCUGUGGGCA | |
| | CaG*u*g | | | GaGg*u*c | | | GUG | |
| 1025 | c*u*UcUcU | 2601 | 2277-2297 | (VPmA)*G*cUu | 1027 | 2275-2297 | CCCUUCUCUA | 3742 |
| | aAgCgCaUc | | | GaUgCgCuUaGa | | | AGCGCAUCAA | |
| | AaG*c*u | | | GaAg*g*g | | | GCU | |
| 1026 | g*c*AgUgC | 2602 | 531-551 | (VPmG)*G*gAa | 1028 | 529-551 | GGGCAGUGCC | 3743 |
| | cAgAuGaAc | | | GuUcAuCuGgCa | | | AGAUGAACUU | |
| | UuC*c*c | | | CuGc*c*c | | | CCC | |
| 1027 | u*u*CaUaU | 2603 | 1073-1093 | (VPmG)*G*uUa | 1029 | 1071-1093 | GAUUCAUAUU | 3744 |
| | uGcAgAcAa | | | UuGuCuGcAaUa | | | GCAGACAAUA | |
| | UaA*c*c | | | UgAa*u*c | | | ACC | |
| 1028 | g*a*AgAcG | 2604 | 1656-1676 | (VPmG)*A*uUu | 1030 | 1654-1676 | AUGAAGACGC | 3745 |
| | cAcAuGcAc | | | GuGcAuGuGcGu | | | ACAUGCACAA | |
| | AaA*u*c | | | CuUc*a*u | | | AUC | |
| 1029 | u*g*GcAgG | 2605 | 1349-1369 | (VPmG)*G*cUa | 1031 | 1347-1369 | GCUGGCAGGG | 3746 |
| | gAaCaCgUc | | | GaCgUgUuCcCu | | | AACACGUCUA | |
| | UaG*c*c | | | GcCa*g*c | | | GCC | |
| 1030 | c*u*AuGgA | 2606 | 1304-1324 | (VPmU)*C*aUg | 1032 | 1302-1324 | GGCUAUGGAG | 3747 |
| | gCcUcCcGc | | | GcGgGaGgCuCc | | | CCUCCCGCCA | |
| | CaU*g*g | | | AuAg*c*c | | | UGG | |
| 1031 | c*a*CgCcA | 2607 | 699-719 | (VPmU)*A*aUc | 1033 | 697-719 | GUCACGCCAG | 3748 |
| | gAgGaUgAc | | | GuCaUcCuCuGg | | | AGGAUGACGA | |
| | GaU*u*g | | | CgUg*a*c | | | UUG | |
| 1032 | c*c*AgCcC | 2608 | 1391-1411 | (VPmG)*U*aAc | 1034 | 1389-1411 | GCCCAGCCCU | 3749 |
| | uAuGcAaAg | | | CuUuGcAuAgGg | | | AUGCAAAGGU | |
| | GuU*a*c | | | CuGg*g*c | | | UAC | |
| 1033 | c*c*AgAcC | 2609 | 475-495 | (VPmU)*C*cCa | 1035 | 473-495 | AACCAGACCA | 3750 |
| | aCgGcCcGu | | | AcGgGcCgUgGu | | | CGGCCCGUUG | |
| | UgG*g*a | | | CuGg*u*u | | | GGA | |
| 1034 | c*u*CuAaG | 2610 | 2280-2300 | (VPmU)*C*gAg | 1036 | 2278-2300 | UUCUCUAAGC | 3751 |
| | cGcAuCaAg | | | CuUgAuGcGcUu | | | GCAUCAAGCU | |
| | CuC*g*a | | | AgAg*a*a | | | CGA | |
| 1035 | g*g*AcAcU | 2611 | 2130-2150 | (VPmG)*A*gUc | 1037 | 2128-2150 | AGGGACACUU | 3752 |
| | uGcGaCgAa | | | UuCgUcGcAaGu | | | GCGACGAAGA | |
| | GaC*u*c | | | GuCc*c*u | | | CUC | |
| 1036 | g*c*CaCaG | 2612 | 2123-2143 | (VPmU)*G*uCg | 1038 | 2121-2143 | GGGCCACAGG | 3753 |
| | gGaCaCuUg | | | CaAgUgUcCcUg | | | GACACUUGCG | |
| | CgA*c*g | | | UgGc*c*c | | | ACG | |
| 1037 | u*c*CcGuG | 2613 | 675-695 | (VPmU)*G*gAu | 1039 | 673-695 | AAUCCCGUGG | 3754 |
| | gAgGuUgGc | | | GcCaAcCuCcAc | | | AGGUUGGCAU | |
| | AuC*c*a | | | GgGa*u*u | | | CCA | |
| 1038 | c*u*GgGcG | 2614 | 2080-2100 | (VPmG)*C*gCu | 1040 | 2078-2100 | UCCUGGGCGA | 3755 |
| | aGaAgCaUa | | | UaUgCuUcUcGc | | | GAAGCAUAAG | |
| | AgC*g*c | | | CcAg*g*a | | | CGC | |
| 1039 | c*a*AcAcG | 2615 | 2334-2354 | (VPmG)*A*gUa | 1041 | 2332-2354 | CCCAACACGG | 3756 |
| | gAgAaCgUg | | | CaCgUuCuCcGu | | | AGAACGUGUA | |
| | UaC*u*c | | | GuUg*g*g | | | CUC | |
| 1040 | a*c*CaCgG | 2616 | 479-499 | (VPmG)*A*gCu | 1042 | 477-499 | AGACCACGGC | 3757 |
| | cCcGuUgGg | | | CcCaAcGgGcCg | | | CCGUUGGGAG | |
| | AgC*u*c | | | UgGu*c*u | | | CUC | |
| 1041 | u*c*CaGgU | 2617 | 692-712 | (VPmU)*A*uCc | 1043 | 690-712 | CAUCCAGGUC | 3758 |
| | CAcGcCaGa | | | UcUgGcGuGaCc | | | ACGCCAGAGG | |
| | GgA*u*g | | | UgGa*u*g | | | AUG | |
| 1042 | u*a*GaUgA | 2618 | 5933-5953 | (VPmG)*U*aAu | 1044 | 5931-5953 | UUUAGAUGAC | 3759 |
| | cCaAaGgUc | | | GaCcUuUgGuCa | | | CAAAGGUCAU | |
| | AuU*a*c | | | UcUa*a*a | | | UAC | |
| 1043 | g*a*AgCaU | 2619 | 2088-2108 | (VPmA)*G*gUg | 1045 | 2086-2108 | GAGAAGCAUA | 3760 |
| | aAgCgCgGc | | | GcCgCgCuUaUg | | | AGCGCGGCCA | |
| | CaC*c*u | | | CuUc*u*c | | | CCU | |
| 1044 | a*a*GuGcA | 2620 | 1600-1620 | (VPmG)*U*gGu | 1046 | 1598-1620 | ACAAGUGCAA | 3761 |
| | aCcUgUgCg | | | CgCaCaGgUuGc | | | CCUGUGCGAC | |
| | AcC*a*c | | | AcUu*g*u | | | CAC | |
| 1045 | a*g*UgCaA | 2621 | 1601-1621 | (VPmU)*G*uGg | 1047 | 1599-1621 | CAAGUGCAAC | 3762 |
| | cCuGuGcGa | | | UcGcAcAgGuUg | | | CUGUGCGACC | |
| | CcA*c*g | | | CaCu*u*g | | | ACG | |
| 1046 | c*c*UgCcC | 2622 | 2010-2030 | (VPmU)*C*cUg | 1048 | 2008-2030 | GCCCUGCCCG | 3763 |
| | gAcGuCaUg | | | CaUgAcGuCgGg | | | ACGUCAUGCA | |
| | CaG*g*g | | | CaGg*g*c | | | GGG | |
| 1047 | c*u*CcUgA | 2623 | 18-38 | (VPmU)*G*cGa | 1049 | 16-38 | GACUCCUGAC | 3764 |
| | cGuUcAaGu | | | AcUuGaAcGuCa | | | GUUCAAGUUC | |
| | UcG*c*a | | | GgAg*u*c | | | GCA | |
| 1048 | g*u*GcAaC | 2624 | 1602-1622 | (VPmG)*C*gUg | 1050 | 1600-1622 | AAGUGCAACC | 3765 |
| | cUgUgCgAc | | | GuCgCaCaGgUu | | | UGUGCGACCA | |
| | CaC*g*c | | | GcAc*u*u | | | CGC | |
| 1049 | g*g*UcCuG | 2625 | 2076-2096 | (VPmU)*U*aUg | 1051 | 2074-2096 | CAGGUCCUGG | 3766 |
| | gGcGaGaAg | | | CuUcUcGcCcAg | | | GCGAGAAGCA | |
| | CaU*a*a | | | GaCc*u*g | | | UAA | |
| 1050 | g*g*CcAcA | 2626 | 2122-2142 | (VPmG)*U*cGc | 1052 | 2120-2142 | AGGGCCACAG | 3767 |
| | gGgAcAcUu | | | AaGuGuCcCuGu | | | GGACACUUGC | |
| | GcG*a*c | | | GgCc*c*u | | | GAC | |
| 1051 | a*g*AaUaC | 2627 | 1108-1128 | (VPmU)*G*aUa | 1053 | 1106-1128 | UAAGAAUACC | 3768 |
| | cAgGaUcAg | | | CuGaUcCuGgUa | | | AGGAUCAGUA | |
| | UaU*c*g | | | UuCu*u*a | | | UCG | |
| 1052 | g*a*AcCaG | 2628 | 472-492 | (VPmU)*A*aCg | 1054 | 470-492 | AUGAACCAGA | 3769 |
| | aCcAcGgCc | | | GgCcGuGgUcUg | | | CCACGGCCCG | |
| | CgU*u*g | | | GuUc*a*u | | | UUG | |
| 1053 | g*g*AgCcU | 2629 | 1308-1328 | (VPmA)*A*aUc | 1055 | 1306-1328 | AUGGAGCCUC | 3770 |
| | cCcGcCaUg | | | CaUgGcGgGaGg | | | CCGCCAUGGA | |
| | GaU*u*u | | | CuCc*a*u | | | UUU | |
| 1054 | u*g*AaCcA | 2630 | 471-491 | (VPmA)*A*cGg | 1056 | 469-491 | GAUGAACCAG | 3552 |
| | gAcCaCgGc | | | GcCgUgGuCuGg | | | ACCACGGCCC | |
| | CcG*u*u | | | UuCa*u*c | | | GUU | |
| 1055 | a*g*CcAgG | 2631 | 1424-1444 | (VPmA)*G*gGc | 1057 | 1422-1444 | CCAGCCAGGU | 3771 |
| | uAgCaAgCc | | | GgCuUgCuAcCu | | | AGCAAGCCGC | |
| | GcC*c*u | | | GgCu*g*g | | | CCU | |
| 1056 | a*g*AcUcC | 2632 | 15-35 | (VPmG)*A*aCu | 1058 | 13-35 | CCAGACUCCU | 3772 |
| | uGaCgUuCa | | | UgAaCgUcAgGa | | | GACGUUCAAG | |
| | AgU*u*c | | | GuCu*g*g | | | UUC | |
| 1057 | u*c*AcGcC | 2633 | 698-718 | (VPmA)*A*uCg | 1059 | 696-718 | GGUCACGCCA | 3773 |
| | aGaGgAuGa | | | UcAuCcUcUgGc | | | GAGGAUGACG | |
| | CgA*u*u | | | GuGa*c*c | | | AUU | |
| 1058 | g*u*CaCgC | 2634 | 697-717 | (VPmA)*U*cGu | 1060 | 695-717 | AGGUCACGCC | 3774 |
| | cAgAgGaUg | | | CaUcCuCuGgCg | | | AGAGGAUGAC | |
| | AcG*a*u | | | UgAc*c*u | | | GAU | |
| 1059 | u*c*CaAuC | 2635 | 670-690 | (VPmG)*C*cAa | 1061 | 668-690 | CAUCCAAUCC | 3775 |
| | cCgUgGaGg | | | CcUcCaCgGgAu | | | CGUGGAGGUU | |
| | UuG*g*c | | | UgGa*u*g | | | GGC | |
| 1060 | c*a*GaCcA | 2636 | 476-496 | (VPmU)*U*cCc | 1062 | 474-496 | ACCAGACCAC | 3776 |
| | cGgCcCgUu | | | AaCgGgCcGuGg | | | GGCCCGUUGG | |
| | GgG*a*g | | | UcUg*g*u | | | GAG | |
| 1061 | g*c*CaAgU | 2637 | 1786-1806 | (VPmG)*U*uCu | 1063 | 1784-1806 | UGGCCAAGUU | 3556 |
| | uCaAgAgCg | | | CgCuCuUgAaCu | | | CAAGAGCGAG | |
| | AgA*a*c | | | UgGc*c*a | | | AAC | |
| 1062 | a*g*AuGaC | 2638 | 5934-5954 | (VPmU)*G*uAa | 1064 | 5932-5954 | UUAGAUGACC | 3778 |
| | cAaAgGuCa | | | UgAcCuUuGgUc | | | AAAGGUCAUU | |
| | UuA*c*a | | | AuCu*a*a | | | ACA | |
| 1063 | u*c*CcUuC | 2639 | 2397-2417 | (VPmU)*C*uCc | 1065 | 2395-2417 | GAUCCCUUCC | 3779 |
| | CUuAgCuUc | | | GaAgCuAaGgAa | | | UUAGCUUCGG | |
| | GgA*g*a | | | GgGa*u*c | | | AGA | |
| 1064 | a*c*AcGuC | 2640 | 1358-1378 | (VPmA)*C*aGc | 1066 | 1356-1378 | GAACACGUCU | 3780 |
| | uAgCcCaCc | | | GgUgGgCuAgAc | | | AGCCCACCGC | |
| | GcU*g*u | | | GuGu*u*c | | | UGU | |
| 1065 | g*u*GgUgG | 2641 | 1780-1800 | (VPmG)*C*uCu | 1067 | 1778-1800 | CCGUGGUGGC | 3551 |
| | cCaAgUuCa | | | UgAaCuUgGcCa | | | CAAGUUCAAG | |
| | AgA*g*c | | | CcAc*g*g | | | AGC | |
| 1066 | c*c*CuUcC | 2642 | 2398-2418 | (VPmG)*U*cUc | 1068 | 2396-2418 | AUCCCUUCCU | 3781 |
| | uUaGcUuCg | | | CgAaGcUaAgGa | | | UAGCUUCGGA | |
| | GaG*a*c | | | AgGg*a*u | | | GAC | |
| 1067 | u*g*CcCaA | 2643 | 2330-2350 | (VPmA)*C*aCg | 1069 | 2328-2350 | GAUGCCCAAC | 3782 |
| | cAcGgAgAa | | | UuCuCcGuGuUg | | | ACGGAGAACG | |
| | CgU*g*u | | | GgCa*u*c | | | UGU | |
| 1068 | g*u*AuCgA | 2644 | 1123-1143 | (VPmG)*C*cGg | 1070 | 1121-1143 | CAGUAUCGAG | 3783 |
| | gAgAgGcUu | | | AaGcCuCuCuCg | | | AGAGGCUUCC | |
| | CcG*g*c | | | AuAc*u*g | | | GGC | |
| 1069 | c*g*UcUaG | 2645 | 1361-1381 | (VPmG)*G*gAc | 1071 | 1359-1381 | CACGUCUAGC | 3784 |
| | cCcAcCgCu | | | AgCgGuGgGcUa | | | CCACCGCUGU | |
| | GuC*c*c | | | GaCg*u*g | | | CCC | |
| 1070 | a*g*GuUgG | 2646 | 683-703 | (VPmG)*C*gUg | 1072 | 681-703 | GGAGGUUGGC | 3785 |
| | CAuCcAgGu | | | AcCuGgAuGcCa | | | AUCCAGGUCA | |
| | CaC*g*c | | | AcCu*c*c | | | CGC | |
| 1071 | u*a*UgGaG | 2647 | 1305-1325 | (VPmU)*C*cAu | 1073 | 1303-1325 | GCUAUGGAGC | 3786 |
| | cCuCcCgCc | | | GgCgGgAgGcUc | | | CUCCCGCCAU | |
| | AuG*g*a | | | CaUa*g*c | | | GGA | |
| 1072 | a*a*UcCcG | 2648 | 673-693 | (VPmG)*A*uGc | 1074 | 671-693 | CCAAUCCCGU | 3787 |
| | uGgAgGuUg | | | CaAcCuCcAcGg | | | GGAGGUUGGC | |
| | GcA*u*c | | | GaUu*g*g | | | AUC | |
| 1073 | u*u*GgCaU | 2649 | 686-706 | (VPmU)*U*gGc | 1075 | 684-706 | GGUUGGCAUC | 3788 |
| | cCaGgUcAc | | | GuGaCcUgGaUg | | | CAGGUCACGC | |
| | GcC*a*g | | | CcAa*c*c | | | CAG | |
| 1074 | c*u*UaGcU | 2650 | 2404-2424 | (VPmU)*C*uGg | 1076 | 2402-2424 | UCCUUAGCUU | 3789 |
| | uCgGaGaCu | | | AgUcUcCgAaGc | | | CGGAGACUCC | |
| | CcA*g*a | | | UaAg*g*a | | | AGA | |
| 1075 | c*a*CgUcU | 2651 | 1359-1379 | (VPmG)*A*cAg | 1077 | 1357-1379 | AACACGUCUA | 3790 |
| | aGcCcAcCg | | | CgGuGgGcUaGa | | | GCCCACCGCU | |
| | CuG*u*c | | | CgUg*u*u | | | GUC | |
| 1076 | a*a*CcAgA | 2652 | 473-493 | (VPmU)*C*aAc | 1078 | 471-493 | UGAACCAGAC | 3791 |
| | cCaCgGcCc | | | GgGcCgUgGuCu | | | CACGGCCCGU | |
| | GuU*g*g | | | GgUu*c*a | | | UGG | |
| 1077 | a*a*CaCgU | 2653 | 1357-1377 | (VPmU)*A*gCg | 1079 | 1355-1377 | GGAACACGUC | 3792 |
| | cUaGcCcAc | | | GuGgGcUaGaCg | | | UAGCCCACCG | |
| | CgC*u*g | | | UgUu*c*c | | | CUG | |
| 1078 | g*u*CcAuC | 2654 | 7-27 | (VPmA)*C*gUc | 1080 | 5-27 | CUGUCCAUCC | 3793 |
| | cAgAcUcCu | | | AgGaGuCuGgAu | | | AGACUCCUGA | |
| | GaC*g*u | | | GgAc*a*g | | | CGU | |
| 1079 | a*g*CuGgA | 2655 | 2498-2518 | (VPmU)*C*gAg | 1081 | 2496-2518 | GGAGCUGGAC | 3794 |
| | cGgAgGgAu | | | AuCcCuCcGuCc | | | GGAGGGAUCU | |
| | CuC*g*g | | | AgCu*c*c | | | CGG | |
| 1080 | c*u*AaGcG | 2656 | 2282-2302 | (VPmU)*C*uCg | 1082 | 2280-2302 | CUCUAAGCGC | 3795 |
| | cAuCaAgCu | | | AgCuUgAuGcGc | | | AUCAAGCUCG | |
| | CgA*g*a | | | UuAg*a*g | | | AGA | |
| 1081 | g*u*UgGcA | 2657 | 685-705 | (VPmU)*G*gCg | 1083 | 683-705 | AGGUUGGCAU | 3796 |
| | uCcAgGuCa | | | UgAcCuGgAuGc | | | CCAGGUCACG | |
| | CgC*c*a | | | CaAc*c*u | | | CCA | |
| 1082 | c*c*AgGuA | 2658 | 1426-1446 | (VPmG)*A*aGg | 1084 | 1424-1446 | AGCCAGGUAG | 3797 |
| | gCaAgCcGc | | | GcGgCuUgCuAc | | | CAAGCCGCCC | |
| | CcU*u*c | | | CuGg*c*u | | | UUC | |
| 1083 | a*c*CuCuC | 2659 | 1059-1079 | (VPmA)*U*aUg | 1085 | 1057-1079 | CCACCUCUCC | 3798 |
| | cAuGgGaUu | | | AaUcCcAuGgAg | | | AUGGGAUUCA | |
| | CaU*a*u | | | AgGu*g*g | | | UAU | |
| 1084 | c*c*AgCcA | 2660 | 1422-1442 | (VPmG)*G*cGg | 1086 | 1420-1442 | UUCCAGCCAG | 3799 |
| | gGuAgCaAg | | | CuUgCuAcCuGg | | | GUAGCAAGCC | |
| | CcG*c*c | | | CuGg*a*a | | | GCC | |
| 1085 | a*g*GgAcA | 2661 | 2128-2148 | (VPmG)*U*cUu | 1087 | 2126-2148 | ACAGGGACAC | 3800 |
| | cUuGcGaCg | | | CgUcGcAaGuGu | | | UUGCGACGAA | |
| | AaG*a*c | | | CcCu*g*u | | | GAC | |
| 1086 | a*c*AaGuG | 2662 | 1598-1618 | (VPmG)*G*uCg | 1088 | 1596-1618 | CUACAAGUGC | 3801 |
| | cAaCcUgUg | | | CaCaGgUuGcAc | | | AACCUGUGCG | |
| | CgA*c*c | | | UuGu*a*g | | | ACC | |
| 1087 | g*c*UgGaC | 2663 | 2499-2519 | (VPmU)*C*cGa | 1089 | 2497-2519 | GAGCUGGACG | 3802 |
| | gGaGgGaUc | | | GaUcCcUcCgUc | | | GAGGGAUCUC | |
| | UcG*g*g | | | CaGc*u*c | | | GGG | |
| 1088 | c*a*AaUuU | 2664 | 1542-1562 | (VPmA)*C*cAg | 1090 | 1540-1562 | UUCAAAUUUC | 3803 |
| | cAgAgCaAc | | | GuUgCuCuGaAa | | | AGAGCAACCU | |
| | CuG*g*u | | | UuUg*a*a | | | GGU | |
| 1089 | c*u*GuCcA | 2665 | 5-25 | (VPmG)*U*cAg | 1091 | 3-25 | CUCUGUCCAU | 3804 |
| | uCcAgAcUc | | | GaGuCuGgAuGg | | | CCAGACUCCU | |
| | CuG*a*c | | | AcAg*a*g | | | GAC | |
| 1090 | a*u*CcCgU | 2666 | 674-694 | (VPmG)*G*aUg | 1092 | 672-694 | CAAUCCCGUG | 3805 |
| | gGaGgUuGg | | | CcAaCcUcCaCg | | | GAGGUUGGCA | |
| | CaU*c*c | | | GgAu*u*g | | | UCC | |
| 1091 | u*c*CuGgG | 2667 | 2078-2098 | (VPmG)*C*uUa | 1093 | 2076-2098 | GGUCCUGGGC | 3557 |
| | cGaGaAgCa | | | UgCuUcUcGcCc | | | GAGAAGCAUA | |
| | UaA*g*c | | | AgGa*c*c | | | AGC | |
| 1092 | u*a*GcUuC | 2668 | 2406-2426 | (VPmU)*G*uCu | 1094 | 2404-2426 | CUUAGCUUCG | 3806 |
| | gGaGaCuCc | | | GgAgUcUcCgAa | | | GAGACUCCAG | |
| | AgA*c*a | | | GcUa*a*g | | | ACA | |
| 1093 | u*a*UcGaG | 2669 | 1124-1144 | (VPmG)*G*cCg | 1095 | 1122-1144 | AGUAUCGAGA | 3807 |
| | aGaGgCuUc | | | GaAgCcUcUcUc | | | GAGGCUUCCG | |
| | CgG*c*c | | | GaUa*c*u | | | GCC | |
| 1094 | c*c*GaCgU | 2670 | 2015-2035 | (VPmU)*C*aUg | 1096 | 2013-2035 | GCCCGACGUC | 3808 |
| | cAuGcAgGg | | | CcCuGcAuGaCg | | | AUGCAGGGCA | |
| | CaU*g*g | | | UcGg*g*c | | | UGG | |
| 1095 | u*g*UcCaU | 2671 | 6-26 | (VPmU)*G*uCa | 1097 | 4-26 | UCUGUCCAUC | 3809 |
| | cCaGaCuCc | | | GgAgUcUgGaUg | | | CAGACUCCUG | |
| | UgA*c*g | | | GaCa*g*a | | | ACG | |
| 1096 | a*g*GgCcA | 2672 | 2120-2140 | (VPmU)*G*cAa | 1098 | 2118-2140 | CGAGGGCCAC | 3810 |
| | cAgGgAcAc | | | GuGuCcCuGuGg | | | AGGGACACUU | |
| | UuG*c*g | | | CcCu*c*g | | | GCG | |
| 1097 | a*c*CaGaC | 2673 | 474-494 | (VPmU)*C*cAa | 1099 | 472-494 | GAACCAGACC | 3811 |
| | cAcGgCcCg | | | CgGgCcGuGgUc | | | ACGGCCCGUU | |
| | UuG*g*g | | | UgGu*u*c | | | GGG | |
| 1098 | g*c*AuAaG | 2674 | 2091-2111 | (VPmG)*C*cAg | 1100 | 2089-2111 | AAGCAUAAGC | 3812 |
| | cGcGgCcAc | | | GuGgCcGcGcUu | | | GCGGCCACCU | |
| | CuG*g*c | | | AuGc*u*u | | | GGC | |
| 1099 | a*u*GgCuA | 2675 | 1300-1320 | (VPmG)*G*cGg | 1101 | 1298-1320 | CAAUGGCUAU | 3813 |
| | uGgAgCcUc | | | GaGgCuCcAuAg | | | GGAGCCUCCC | |
| | CcG*c*c | | | CcAu*u*g | | | GCC | |
| 1100 | C*a*GgAaC | 2676 | 754-774 | (VPmU)*U*uAu | 1102 | 752-774 | AACAGGAACA | 3814 |
| | aCaUaGcAg | | | CuGcUaUgUgUu | | | CAUAGCAGAU | |
| | AuA*a*A | | | CcUg*U*u | | | AAA | |
| 1101 | A*g*GaAcA | 2677 | 755-775 | (VPmG)*U*uUa | 1103 | 753-775 | ACAGGAACAC | 3815 |
| | cAuAgCaGa | | | UcUgCuAuGuGu | | | AUAGCAGAUA | |
| | UaA*a*C | | | UcCu*G*u | | | AAC | |
| 1102 | G*g*AaCaC | 2678 | 756-776 | (VPmA)*G*uUu | 1104 | 754-776 | CAGGAACACA | 3816 |
| | aUaGcAgAu | | | AuCuGcUaUgUg | | | UAGCAGAUAA | |
| | AaA*c*U | | | UuCc*U*g | | | ACU | |
| 1103 | G*a*AcAcA | 2679 | 757-777 | (VPmA)*A*gUu | 1105 | 755-777 | AGGAACACAU | 3817 |
| | uAgCaGaUa | | | UaUcUgCuAuGu | | | AGCAGAUAAA | |
| | AaC*u*U | | | GuUc*C*u | | | CUU | |
| 1104 | A*a*CaCaU | 2680 | 758-778 | (VPmG)*A*aGu | 1106 | 756-778 | GGAACACAUA | 3818 |
| | aGcAgAuAa | | | UuAuCuGcUaUg | | | GCAGAUAAAC | |
| | AcU*u*C | | | UgUu*C*c | | | UUC | |
| 1105 | C*c*CaGcA | 2681 | 883-903 | (VPmU)*G*uAc | 1107 | 881-903 | AGCCCAGCAG | 3819 |
| | gCuAcAcAu | | | AuGuGuAgCuGc | | | CUACACAUGU | |
| | GuA*c*A | | | UgGg*C*u | | | ACA | |
| 1106 | C*a*GcAgC | 2682 | 885-905 | (VPmG)*U*uGu | 1108 | 883-905 | CCCAGCAGCU | 3820 |
| | uAcAcAuGu | | | AcAuGuGuAgCu | | | ACACAUGUAC | |
| | AcA*a*C | | | GcUg*G*g | | | AAC | |
| 1107 | G*c*AgCuA | 2683 | 887-907 | (VPmA)*A*gUu | 1109 | 885-907 | CAGCAGCUAC | 3821 |
| | CACAuGuAc | | | GuAcAuGuGuAg | | | ACAUGUACAA | |
| | AaC*u*U | | | CuGc*U*g | | | CUU | |
| 1108 | A*g*CuAcA | 2684 | 889-909 | (VPmG)*C*aAg | 1110 | 887-909 | GCAGCUACAC | 3822 |
| | cAuGuAcAa | | | UuGuAcAuGuGu | | | AUGUACAACU | |
| | CuU*g*C | | | AgCu*G*c | | | UGC | |
| 1109 | C*u*AcAcA | 2685 | 891-911 | (VPmU)*U*gCa | 1111 | 889-911 | AGCUACACAU | 3823 |
| | uGuAcAaCu | | | AgUuGuAcAuGu | | | GUACAACUUG | |
| | UgC*a*A | | | GuAg*C*u | | | CAA | |
| 1110 | U*a*CaCaU | 2686 | 892-912 | (VPmU)*U*uGc | 1112 | 890-912 | GCUACACAUG | 3824 |
| | gUaCaAcUu | | | AaGuUgUaCaUg | | | UACAACUUGC | |
| | GcA*a*A | | | UgUa*G*c | | | AAA | |
| 1111 | A*c*AuGuA | 2687 | 895-915 | (VPmA)*U*gUu | 1113 | 893-915 | ACACAUGUAC | 3825 |
| | cAaCuUgCa | | | UgCaAgUuGuAc | | | AACUUGCAAA | |
| | AaC*a*U | | | AuGu*G*u | | | CAG | |
| 1112 | C*a*UgUaC | 2688 | 896-916 | (VPmG)*C*uGu | 1114 | 894-916 | CACAUGUACA | 3826 |
| | aAcUuGcAa | | | UuGcAaGuUgUa | | | ACUUGCAAAC | |
| | AcA*g*C | | | CaUg*U*g | | | AGC | |
| 1113 | A*u*GuAcA | 2689 | 897-917 | (VPmG)*G*cUg | 1115 | 895-917 | ACAUGUACAA | 3827 |
| | aCuUgCaAa | | | UuUgCaAgUuGu | | | CUUGCAAACA | |
| | CaG*c*C | | | AcAu*G*u | | | GCC | |
| 1114 | U*g*UaCaA | 2690 | 898-918 | (VPmU)*G*gCu | 1116 | 896-918 | CAUGUACAAC | 3828 |
| | CUuGcAaAc | | | GuUuGcAaGuUg | | | UUGCAAACAG | |
| | AgC*c*A | | | UaCa*U*g | | | CCA | |
| 1115 | G*u*AcAaC | 2691 | 899-919 | (VPmA)*U*gGc | 1117 | 897-919 | AUGUACAACU | 3829 |
| | uUgCaAaCa | | | UgUuUgCaAgUu | | | UGCAAACAGC | |
| | GcC*a*U | | | GuAc*A*u | | | CAU | |
| 1116 | U*a*CaAcU | 2692 | 900-920 | (VPmA)*A*uGg | 1118 | 898-920 | UGUACAACUU | 3830 |
| | uGcAaAcAg | | | CuGuUuGcAaGu | | | GCAAACAGCC | |
| | CcA*u*U | | | UgUa*C*a | | | AUU | |
| 1117 | A*c*AaCuU | 2693 | 901-921 | (VPmG)*A*aUg | 1119 | 899-921 | GUACAACUUG | 3831 |
| | gCaAaCaGc | | | GcUgUuUgCaAg | | | CAAACAGCCA | |
| | CaU*u*C | | | UuGu*A*c | | | UUC | |
| 1118 | C*a*AcUuG | 2694 | 902-922 | (VPmU)*G*aAu | 1120 | 900-922 | UACAACUUGC | 3832 |
| | CAaAcAgCc | | | GgCuGuUuGcAa | | | AAACAGCCAU | |
| | AuU*c*A | | | GuUg*U*a | | | UCA | |
| 1119 | A*a*CuUgC | 2695 | 903-923 | (VPmG)*U*gAa | 1121 | 901-923 | ACAACUUGCA | 3833 |
| | aAaCaGcCa | | | UgGcUgUuUgCa | | | AACAGCCAUU | |
| | UuC*a*C | | | AgUu*G*u | | | CAC | |
| 1120 | A*c*UuGcA | 2696 | 904-924 | (VPmG)*G*uGa | 1122 | 902-924 | CAACUUGCAA | 3834 |
| | aAcAgCcAu | | | AuGgCuGuUuGc | | | ACAGCCAUUC | |
| | UcA*c*C | | | AaGu*U*g | | | ACC | |
| 1121 | C*u*UgCaA | 2697 | 905-925 | (VPmU)*G*gUg | 1123 | 903-925 | AACUUGCAAA | 3835 |
| | aCaGcCaUu | | | AaUgGcUgUuUg | | | CAGCCAUUCA | |
| | CaC*c*A | | | CaAg*U*u | | | CCA | |
| 1122 | U*u*GcAaA | 2698 | 906-926 | (VPmA)*U*gGu | 1124 | 904-926 | ACUUGCAAAC | 3836 |
| | cAgCcAuUc | | | GaAuGgCuGuUu | | | AGCCAUUCAC | |
| | AcC*a*U | | | GcAa*G*u | | | CAG | |
| 1123 | U*g*CaAaC | 2699 | 907-927 | (VPmA)*C*uGg | 1125 | 905-927 | CUUGCAAACA | 3837 |
| | aGcCaUuCa | | | UgAaUgGcUgUu | | | GCCAUUCACC | |
| | CcA*g*U | | | UgCa*A*g | | | AGU | |
| 1124 | G*c*AaAcA | 2700 | 908-928 | (VPmA)*A*cUg | 1126 | 906-928 | UUGCAAACAG | 3838 |
| | gCcAuUcAc | | | GuGaAuGgCuGu | | | CCAUUCACCA | |
| | CaG*u*U | | | UuGc*A*a | | | GUG | |
| 1125 | C*a*AaCaG | 2701 | 909-929 | (VPmG)*C*aCu | 1127 | 907-929 | UGCAAACAGC | 3839 |
| | cCaUuCaCc | | | GgUgAaUgGcUg | | | CAUUCACCAG | |
| | AgU*g*C | | | UuUg*C*a | | | UGC | |
| 1126 | A*a*AcAgC | 2702 | 910-930 | (VPmU)*G*cAc | 1128 | 908-930 | GCAAACAGCC | 3840 |
| | cAuUcAcCa | | | UgGuGaAuGgCu | | | AUUCACCAGU | |
| | GuG*c*A | | | GuUu*G*c | | | GCA | |
| 1127 | A*a*CaGcC | 2703 | 911-931 | (VPmA)*U*gCa | 1129 | 909-931 | CAAACAGCCA | 3841 |
| | aUuCaCcAg | | | CuGgUgAaUgGc | | | UUCACCAGUG | |
| | UgC*a*U | | | UgUu*U*g | | | CAU | |
| 1128 | A*c*AgCcA | 2704 | 912-932 | (VPmA)*A*uGc | 1130 | 910-932 | AAACAGCCAU | 3842 |
| | uUcAcCaGu | | | AcUgGuGaAuGg | | | UCACCAGUGC | |
| | GcA*u*U | | | CuGu*U*u | | | AUG | |
| 1129 | C*a*GcCaU | 2705 | 913-933 | (VPmA)*C*aUg | 1131 | 911-933 | AACAGCCAUU | 3843 |
| | uCaCcAgUg | | | CaCuGgUgAaUg | | | CACCAGUGCA | |
| | CaU*g*U | | | GcUg*U*u | | | UGG | |
| 1130 | A*g*CcAuU | 2706 | 914-934 | (VPmA)*C*cAu | 1132 | 912-934 | ACAGCCAUUC | 3844 |
| | cAcCaGuGc | | | GcAcUgGuGaAu | | | ACCAGUGCAU | |
| | AuG*g*U | | | GgCu*G*u | | | GGU | |
| 1131 | G*c*CaUuC | 2707 | 915-935 | (VPmA)*A*cCa | 1133 | 913-935 | CAGCCAUUCA | 3845 |
| | aCcAgUgCa | | | UgCaCuGgUgAa | | | CCAGUGCAUG | |
| | UgG*u*U | | | UgGc*U*g | | | GUU | |
| 1132 | C*c*AuUcA | 2708 | 916-936 | (VPmA)*A*aCc | 1134 | 914-936 | AGCCAUUCAC | 3846 |
| | cCaGuGcAu | | | AuGcAcUgGuGa | | | CAGUGCAUGG | |
| | GgU*u*U | | | AuGg*C*u | | | UUU | |
| 1133 | C*a*UuCaC | 2709 | 917-937 | (VPmG)*A*aAc | 1135 | 915-937 | GCCAUUCACC | 3847 |
| | cAgUgCaUg | | | CaUgCaCuGgUg | | | AGUGCAUGGU | |
| | GuU*u*C | | | AaUg*G*c | | | UUC | |
| 1134 | A*u*UcAcC | 2710 | 918-938 | (VPmA)*G*aAa | 1136 | 916-938 | CCAUUCACCA | 3848 |
| | aGuGcAuGg | | | CcAuGcAcUgGu | | | GUGCAUGGUU | |
| | UuU*c*U | | | GaAu*G*g | | | UCU | |
| 1135 | U*u*CaCcA | 2711 | 919-939 | (VPmG)*A*gAa | 1137 | 917-939 | CAUUCACCAG | 3849 |
| | gUgCaUgGu | | | AcCaUgCaCuGg | | | UGCAUGGUUU | |
| | UuC*u*C | | | UgAa*U*g | | | CUC | |
| 1136 | U*c*AcCaG | 2712 | 920-940 | (VPmA)*G*aGa | 1138 | 918-940 | AUUCACCAGU | 3850 |
| | uGcAuGgUu | | | AaCcAuGcAcUg | | | GCAUGGUUUC | |
| | UcU*c*U | | | GuGa*A*u | | | UCU | |
| 1137 | C*a*CcAgU | 2713 | 921-941 | (VPmA)*A*gAg | 1139 | 919-941 | UUCACCAGUG | 3851 |
| | gCaUgGuUu | | | AaAcCaUgCaCu | | | CAUGGUUUCU | |
| | CuC*u*U | | | GgUg*A*a | | | CUU | |
| 1138 | A*c*CaGuG | 2714 | 922-942 | (VPmA)*A*aGa | 1140 | 920-942 | UCACCAGUGC | 3852 |
| | cAuGgUuUc | | | GaAaCcAuGcAc | | | AUGGUUUCUC | |
| | UcU*u*U | | | UgGu*G*a | | | UUG | |
| 1139 | A*g*UgCaU | 2715 | 925-945 | (VPmU)*U*gCa | 1141 | 923-945 | CCAGUGCAUG | 3853 |
| | gGuUuCuCu | | | AgAgAaAcCaUg | | | GUUUCUCUUG | |
| | UgC*a*A | | | CaCu*G*g | | | CAA | |
| 1140 | G*g*UuUcU | 2716 | 932-952 | (VPmG)*U*gCg | 1142 | 930-952 | AUGGUUUCUC | 3854 |
| | CUuGcAaCa | | | UgUuGcAaGaGa | | | UUGCAACACG | |
| | CgC*a*C | | | AaCc*A*u | | | CAC | |
| 1141 | G*u*UuCuC | 2717 | 933-953 | (VPmU)*G*uGc | 1143 | 931-953 | UGGUUUCUCU | 3855 |
| | uUgCaAcAc | | | GuGuUgCaAgAg | | | UGCAACACGC | |
| | GcA*C*A | | | AaAc*C*a | | | ACA | |
| 1142 | U*u*CuCuU | 2718 | 935-955 | (VPmU)*C*uGu | 1144 | 933-955 | GUUUCUCUUG | 3856 |
| | gCaAcAcGc | | | GcGuGuUgCaAg | | | CAACACGCAC | |
| | AcA*g*A | | | AgAa*A*c | | | AGA | |
| 1143 | U*c*UcUuG | 2719 | 936-956 | (VPmU)*U*cUg | 1145 | 934-956 | UUUCUCUUGC | 3857 |
| | cAaCaCgCa | | | UgCgUgUuGcAa | | | AACACGCACA | |
| | CaG*a*A | | | GaGa*A*a | | | GAA | |
| 1144 | C*u*UgCaA | 2720 | 939-959 | (VPmG)*U*gUu | 1146 | 937-959 | CUCUUGCAAC | 3858 |
| | CAcGcAcAg | | | CuGuGcGuGuUg | | | ACGCACAGAA | |
| | AaC*a*C | | | CaAg*A*g | | | CAC | |
| 1145 | U*u*GcAaC | 2721 | 940-960 | (VPmA)*G*uGu | 1147 | 938-960 | UCUUGCAACA | 3859 |
| | aCgCaCaGa | | | UcUgUgCgUgUu | | | CGCACAGAAC | |
| | AcA*c*U | | | GcAa*G*a | | | ACU | |
| 1146 | U*g*CaAcA | 2722 | 941-961 | (VPmG)*A*gUg | 1148 | 939-961 | CUUGCAACAC | 3860 |
| | cGcAcAgAa | | | UuCuGuGcGuGu | | | GCACAGAACA | |
| | CaC*u*C | | | UgCa*A*g | | | CUC | |
| 1147 | G*c*AaCaC | 2723 | 942-962 | (VPmU)*G*aGu | 1149 | 940-962 | UUGCAACACG | 3861 |
| | gCaCaGaAc | | | GuUcUgUgCgUg | | | CACAGAACAC | |
| | AcU*c*A | | | UuGc*A*a | | | UCA | |
| 1148 | C*a*AcAcG | 2724 | 943-963 | (VPmA)*U*gAg | 1150 | 941-963 | UGCAACACGC | 3862 |
| | cAcAgAaCa | | | UgUuCuGuGcGu | | | ACAGAACACU | |
| | CuC*a*U | | | GuUg*C*a | | | CAU | |
| 1149 | A*c*AcGcA | 2725 | 945-965 | (VPmA)*C*aUg | 1151 | 943-965 | CAACACGCAC | 3863 |
| | cAgAaCaCu | | | AgUgUuCuGuGc | | | AGAACACUCA | |
| | CaU*g*U | | | GuGu*U*g | | | UGG | |
| 1150 | C*a*CgCaC | 2726 | 946-966 | (VPmU)*C*cAu | 1152 | 944-966 | AACACGCACA | 3864 |
| | aGaAcAcUc | | | GaGuGuUcUgUg | | | GAACACUCAU | |
| | AuG*g*A | | | CgUg*U*u | | | GGA | |
| 1151 | A*c*GcAcA | 2727 | 947-967 | (VPmA)*U*cCa | 1153 | 945-967 | ACACGCACAG | 3865 |
| | gAaCaCuCa | | | UgAgUgUuCuGu | | | AACACUCAUG | |
| | UgG*a*U | | | GcGu*G*u | | | GAU | |
| 1152 | C*g*CaCaG | 2728 | 948-968 | (VPmA)*A*uCc | 1154 | 946-968 | CACGCACAGA | 3866 |
| | aAcAcUcAu | | | AuGaGuGuUcUg | | | ACACUCAUGG | |
| | GgA*u*U | | | UgCg*U*g | | | AUU | |
| 1153 | G*c*AcAgA | 2729 | 949-969 | (VPmU)*A*aUc | 1155 | 947-969 | ACGCACAGAA | 3867 |
| | aCaCuCaUg | | | CaUgAgUgUuCu | | | CACUCAUGGA | |
| | GaU*u*A | | | GuGc*G*u | | | UUA | |
| 1154 | C*a*CaGaA | 2730 | 950-970 | (VPmU)*U*aAu | 1156 | 948-970 | CGCACAGAAC | 3868 |
| | cAcUcAuGg | | | CcAuGaGuGuUc | | | ACUCAUGGAU | |
| | AuU*a*A | | | UgUg*C*g | | | UAA | |
| 1155 | A*c*AgAaC | 2731 | 951-971 | (VPmA)*U*uAa | 1157 | 949-971 | GCACAGAACA | 3869 |
| | aCuCaUgGa | | | UcCaUgAgUgUu | | | CUCAUGGAUU | |
| | UuA*a*U | | | CuGu*G*c | | | AAG | |
| 1156 | C*a*GaAcA | 2732 | 952-972 | (VPmU)*C*uUa | 1158 | 950-972 | CACAGAACAC | 3870 |
| | cUcAuGgAu | | | AuCcAuGaGuGu | | | UCAUGGAUUA | |
| | UaA*g*A | | | UcUg*U*g | | | AGA | |
| 1157 | A*g*AaCaC | 2733 | 953-973 | (VPmU)*U*cUu | 1159 | 951-973 | ACAGAACACU | 3871 |
| | uCaUgGaUu | | | AaUcCaUgAgUg | | | CAUGGAUUAA | |
| | AaG*a*A | | | UuCu*G*u | | | GAA | |
| 1158 | G*a*AcAcU | 2734 | 954-974 | (VPmA)*U*uCu | 1160 | 952-974 | CAGAACACUC | 3872 |
| | cAuGgAuUa | | | UaAuCcAuGaGu | | | AUGGAUUAAG | |
| | AgA*a*U | | | GuUc*U*g | | | AAU | |
| 1159 | A*a*CaCuC | 2735 | 955-975 | (VPmG)*A*uUc | 1161 | 953-975 | AGAACACUCA | 3873 |
| | aUgGaUuAa | | | UuAaUcCaUgAg | | | UGGAUUAAGA | |
| | GaA*u*C | | | UgUu*C*u | | | AUC | |
| 1160 | U*u*AaGaA | 2736 | 967-987 | (VPmG)*C*uUu | 1162 | 965-987 | GAUUAAGAAU | 3874 |
| | uCuAcUuAg | | | CuAaGuAgAuUc | | | CUACUUAGAA | |
| | AaA*g*C | | | UuAa*U*c | | | AGC | |
| 1161 | G*g*UuGgU | 2737 | 1014-1034 | (VPmA)*G*uCc | 1163 | 1012-1034 | CGGGUUGGUA | 3875 |
| | aUcCcUuCa | | | UgAaGgGaUaCc | | | UCCCUUCAGG | |
| | GgA*c*U | | | AaCc*C*g | | | ACU | |
| 1162 | G*u*UgGuA | 2738 | 1015-1035 | (VPmU)*A*gUc | 1164 | 1013-1035 | GGGUUGGUAU | 3876 |
| | uCcCuUcAg | | | CuGaAgGgAuAc | | | CCCUUCAGGA | |
| | GaC*u*A | | | CaAc*C*c | | | CUA | |
| 1163 | U*u*GgUaU | 2739 | 1016-1036 | (VPmA)*U*aGu | 1165 | 1014-1036 | GGUUGGUAUC | 3877 |
| | cCcUuCaGg | | | CcUgAaGgGaUa | | | CCUUCAGGAC | |
| | AcU*a*U | | | CcAa*C*c | | | UAG | |
| 1164 | U*g*GuAuC | 2740 | 1017-1037 | (VPmA)*C*uAg | 1166 | 1015-1037 | GUUGGUAUCC | 3878 |
| | cCuUcAgGa | | | UcCuGaAgGgAu | | | CUUCAGGACU | |
| | CuA*g*U | | | AcCa*A*c | | | AGG | |
| 1165 | G*g*UaUcC | 2741 | 1018-1038 | (VPmA)*C*cUa | 1167 | 1016-1038 | UUGGUAUCCC | 3879 |
| | cUuCaGgAc | | | GuCcUgAaGgGa | | | UUCAGGACUA | |
| | UaG*g*U | | | UaCc*A*a | | | GGU | |
| 1166 | A*u*CcCuU | 2742 | 1021-1041 | (VPmU)*G*cAc | 1168 | 1019-1041 | GUAUCCCUUC | 3880 |
| | cAgGaCuAg | | | CuAgUcCuGaAg | | | AGGACUAGGU | |
| | GuG*c*A | | | GgAu*A*c | | | GCA | |
| 1167 | C*c*UuCaG | 2743 | 1024-1044 | (VPmU)*U*cUg | 1169 | 1022-1044 | UCCCUUCAGG | 3881 |
| | gAcUaGgUg | | | CaCcUaGuCcUg | | | ACUAGGUGCA | |
| | CaG*a*A | | | AaGg*G*a | | | GAA | |
| 1168 | U*u*CaGgA | 2744 | 1026-1046 | (VPmA)*A*uUc | 1170 | 1024-1046 | CCUUCAGGAC | 3882 |
| | cUaGgUgCa | | | UgCaCcUaGuCc | | | UAGGUGCAGA | |
| | GaA*u*U | | | UgAa*G*g | | | AUG | |
| 1169 | U*c*AgGaC | 2745 | 1027-1047 | (VPmA)*C*aUu | 1171 | 1025-1047 | CUUCAGGACU | 3883 |
| | uAgGuGcAg | | | CuGcAcCuAgUc | | | AGGUGCAGAA | |
| | AaU*g*U | | | CuGa*A*g | | | UGU | |
| 1170 | C*a*GgAcU | 2746 | 1028-1048 | (VPmG)*A*cAu | 1172 | 1026-1048 | UUCAGGACUA | 3884 |
| | aGgUgCaGa | | | UcUgCaCcUaGu | | | GGUGCAGAAU | |
| | AuG*u*C | | | CcUg*A*a | | | GUC | |
| 1171 | A*g*GaCuA | 2747 | 1029-1049 | (VPmG)*G*aCa | 1173 | 1027-1049 | UCAGGACUAG | 3885 |
| | gGuGcAgAa | | | UuCuGcAcCuAg | | | GUGCAGAAUG | |
| | UgU*c*C | | | UcCu*G*a | | | UCC | |
| 1172 | G*g*AcUaG | 2748 | 1030-1050 | (VPmA)*G*gAc | 1174 | 1028-1050 | CAGGACUAGG | 3886 |
| | gUgCaGaAu | | | AuUcUgCaCcUa | | | UGCAGAAUGU | |
| | GuC*c*U | | | GuCc*U*g | | | CCU | |
| 1173 | G*a*CuAgG | 2749 | 1031-1051 | (VPmA)*A*gGa | 1175 | 1029-1051 | AGGACUAGGU | 3887 |
| | uGcAgAaUg | | | CaUuCuGcAcCu | | | GCAGAAUGUC | |
| | UcC*u*U | | | AgUc*C*u | | | CUU | |
| 1174 | C*a*CcUcU | 2750 | 1058-1078 | (VPmU)*A*uGa | 1176 | 1056-1078 | GCCACCUCUC | 3888 |
| | cCaUgGgAu | | | AuCcCaUgGaGa | | | CAUGGGAUUC | |
| | UcA*u*A | | | GgUg*G*c | | | AUA | |
| 1175 | A*c*CuCuC | 2751 | 1059-1079 | (VPmA)*U*aUg | 1177 | 1057-1079 | CCACCUCUCC | 3889 |
| | cAuGgGaUu | | | AaUcCcAuGgAg | | | AUGGGAUUCA | |
| | CaU*a*U | | | AgGu*G*g | | | UAU | |
| 1176 | C*c*UcUcC | 2752 | 1060-1080 | (VPmA)*A*uAu | 1178 | 1058-1080 | CACCUCUCCA | 3890 |
| | aUgGgAuUc | | | GaAuCcCaUgGa | | | UGGGAUUCAU | |
| | AuA*u*U | | | GaGg*U*g | | | AUU | |
| 1177 | C*u*CuCcA | 2753 | 1061-1081 | (VPmA)*A*aUa | 1179 | 1059-1081 | ACCUCUCCAU | 3891 |
| | uGgGaUuCa | | | UgAaUcCcAuGg | | | GGGAUUCAUA | |
| | UaU*u*U | | | AgAg*G*u | | | UUG | |
| 1178 | U*c*UcCaU | 2754 | 1062-1082 | (VPmG)*C*aAu | 1180 | 1060-1082 | CCUCUCCAUG | 3892 |
| | gGgAuUcAu | | | AuGaAuCcCaUg | | | GGAUUCAUAU | |
| | AuU*g*C | | | GaGa*G*g | | | UGC | |
| 1179 | C*u*CcAuG | 2755 | 1063-1083 | (VPmU)*G*cAa | 1181 | 1061-1083 | CUCUCCAUGG | 3893 |
| | gGaUuCaUa | | | UaUgAaUcCcAu | | | GAUUCAUAUU | |
| | UuG*c*A | | | GgAg*A*g | | | GCA | |
| 1180 | U*c*CaUgG | 2756 | 1064-1084 | (VPmA)*U*gCa | 1182 | 1062-1084 | UCUCCAUGGG | 3894 |
| | gAuUcAuAu | | | AuAuGaAuCcCa | | | AUUCAUAUUG | |
| | UgC*a*U | | | UgGa*G*a | | | CAG | |
| 1181 | C*c*AuGgG | 2757 | 1065-1085 | (VPmU)*C*uGc | 1183 | 1063-1085 | CUCCAUGGGA | 3895 |
| | aUuCaUaUu | | | AaUaUgAaUcCc | | | UUCAUAUUGC | |
| | GcA*g*A | | | AuGg*A*g | | | AGA | |
| 1182 | C*a*UgGgA | 2758 | 1066-1086 | (VPmG)*U*cUg | 1184 | 1064-1086 | UCCAUGGGAU | 3896 |
| | uUcAuAuUg | | | CaAuAuGaAuCc | | | UCAUAUUGCA | |
| | CaG*a*C | | | CaUg*G*a | | | GAC | |
| 1183 | A*u*GgGaU | 2759 | 1067-1087 | (VPmU)*G*uCu | 1185 | 1065-1087 | CCAUGGGAUU | 3897 |
| | uCaUaUuGc | | | GcAaUaUgAaUc | | | CAUAUUGCAG | |
| | AgA*c*A | | | CcAu*G*g | | | ACA | |
| 1184 | U*g*GgAuU | 2760 | 1068-1088 | (VPmU)*U*gUc | 1186 | 1066-1088 | CAUGGGAUUC | 3898 |
| | cAuAuUgCa | | | UgCaAuAuGaAu | | | AUAUUGCAGA | |
| | GaC*a*A | | | CcCa*U*g | | | CAA | |
| 1185 | G*g*GaUuC | 2761 | 1069-1089 | (VPmA)*U*uGu | 1187 | 1067-1089 | AUGGGAUUCA | 3899 |
| | aUaUuGcAg | | | CuGcAaUaUgAa | | | UAUUGCAGAC | |
| | AcA*a*U | | | UcCc*A*u | | | AAU | |
| 1186 | G*g*AuUcA | 2762 | 1070-1090 | (VPmU)*A*uUg | 1188 | 1068-1090 | UGGGAUUCAU | 3900 |
| | uAuUgCaGa | | | UcUgCaAuAuGa | | | AUUGCAGACA | |
| | CaA*u*A | | | AuCc*C*a | | | AUA | |
| 1187 | G*a*UuCaU | 2763 | 1071-1091 | (VPmU)*U*aUu | 1189 | 1069-1091 | GGGAUUCAUA | 3901 |
| | aUuGcAgAc | | | GuCuGcAaUaUg | | | UUGCAGACAA | |
| | AaU*a*A | | | AaUc*C*c | | | UAA | |
| 1188 | A*u*UcAuA | 2764 | 1072-1092 | (VPmG)*U*uAu | 1190 | 1070-1092 | GGAUUCAUAU | 3902 |
| | uUgCaGaCa | | | UgUcUgCaAuAu | | | UGCAGACAAU | |
| | AuA*a*C | | | GaAu*C*c | | | AAC | |
| 1189 | U*u*CaUaU | 2765 | 1073-1093 | (VPmG)*G*uUa | 1191 | 1071-1093 | GAUUCAUAUU | 3903 |
| | uGcAgAcAa | | | UuGuCuGcAaUa | | | GCAGACAAUA | |
| | UaA*c*C | | | UgAa*U*c | | | ACC | |
| 1190 | U*c*AuAuU | 2766 | 1074-1094 | (VPmG)*G*gUu | 1192 | 1072-1094 | AUUCAUAUUG | 3904 |
| | gCaGaCaAu | | | AuUgUcUgCaAu | | | CAGACAAUAA | |
| | AaC*c*C | | | AuGa*A*u | | | CCC | |
| 1191 | C*u*UuAaC | 2767 | 1095-1115 | (VPmG)*G*uAu | 1193 | 1093-1115 | CCCUUUAACC | 3905 |
| | cUgCuAaGa | | | UcUuAgCaGgUu | | | UGCUAAGAAU | |
| | AuA*c*C | | | AaAg*G*g | | | ACC | |
| 1192 | U*u*UaAcC | 2768 | 1096-1116 | (VPmU)*G*gUa | 1194 | 1094-1116 | CCUUUAACCU | 3906 |
| | uGcUaAgAa | | | UuCuUaGcAgGu | | | GCUAAGAAUA | |
| | UaC*c*A | | | UaAa*G*g | | | CCA | |
| 1193 | U*u*AaCcU | 2769 | 1097-1117 | (VPmA)*U*gGu | 1195 | 1095-1117 | CUUUAACCUG | 3907 |
| | gCuAaGaAu | | | AuUcUuAgCaGg | | | CUAAGAAUAC | |
| | AcC*a*U | | | UuAa*A*g | | | CA | |
| 1194 | U*a*AcCuG | 2770 | 1098-1118 | (VPmA)*C*uGg | 1196 | 1096-1118 | UUUAACCUGC | 3908 |
| | cUaAgAaUa | | | UaUuCuUaGcAg | | | UAAGAAUACC | |
| | CcA*g*U | | | GuUa*A*a | | | AGG | |
| 1195 | A*a*CcUgC | 2771 | 1099-1119 | (VPmU)*C*cUg | 1197 | 1097-1119 | UUAACCUGCU | 3909 |
| | uAaGaAuAc | | | GuAuUcUuAgCa | | | AAGAAUACCA | |
| | CaG*g*A | | | GgUu*A*a | | | GGA | |
| 1196 | A*c*CuGcU | 2772 | 1100-1120 | (VPmA)*U*cCu | 1198 | 1098-1120 | UAACCUGCUA | 3910 |
| | aAgAaUaCc | | | GgUaUuCuUaGc | | | AGAAUACCAG | |
| | AgG*a*U | | | AgGu*U*a | | | GAU | |
| 1197 | C*c*UgCuA | 2773 | 1101-1121 | (VPmG)*A*uCc | 1199 | 1099-1121 | AACCUGCUAA | 3911 |
| | aGaAuAcCa | | | UgGuAuUcUuAg | | | GAAUACCAGG | |
| | GgA*u*C | | | CaGg*U*u | | | AUC | |
| 1198 | C*u*GcUaA | 2774 | 1102-1122 | (VPmU)*G*aUc | 1200 | 1100-1122 | ACCUGCUAAG | 3912 |
| | gAaUaCcAg | | | CuGgUaUuCuUa | | | AAUACCAGGA | |
| | GaU*c*A | | | GcAg*G*u | | | UCA | |
| 1199 | U*g*CuAaG | 2775 | 1103-1123 | (VPmA)*U*gAu | 1201 | 1101-1123 | CCUGCUAAGA | 3913 |
| | aAuAcCaGg | | | CcUgGuAuUcUu | | | AUACCAGGAU | |
| | AuC*a*U | | | AgCa*G*g | | | CAG | |
| 1200 | G*c*UaAgA | 2776 | 1104-1124 | (VPmA)*C*uGa | 1202 | 1102-1124 | CUGCUAAGAA | 3914 |
| | aUaCcAgGa | | | UcCuGgUaUuCu | | | UACCAGGAUC | |
| | UcA*g*U | | | UaGc*A*g | | | AGU | |
| 1201 | C*u*AaGaA | 2777 | 1105-1125 | (VPmU)*A*cUg | 1203 | 1103-1125 | UGCUAAGAAU | 3915 |
| | uAcCaGgAu | | | AuCcUgGuAuUc | | | ACCAGGAUCA | |
| | CaG*u*A | | | UuAg*C*a | | | GUA | |
| 1202 | U*a*AgAaU | 2778 | 1106-1126 | (VPmA)*U*aCu | 1204 | 1104-1126 | GCUAAGAAUA | 3916 |
| | aCcAgGaUc | | | GaUcCuGgUaUu | | | CCAGGAUCAG | |
| | AgU*a*U | | | CuUa*G*c | | | UAU | |
| 1203 | A*a*GaAuA | 2779 | 1107-1127 | (VPmG)*A*uAc | 1205 | 1105-1127 | CUAAGAAUAC | 3917 |
| | cCaGgAuCa | | | UgAuCcUgGuAu | | | CAGGAUCAGU | |
| | GuA*u*C | | | UcUu*A*g | | | AUC | |
| 1204 | A*g*AaUaC | 2780 | 1108-1128 | (VPmA)*G*aUa | 1206 | 1106-1128 | UAAGAAUACC | 3918 |
| | cAgGaUcAg | | | CuGaUcCuGgUa | | | AGGAUCAGUA | |
| | UaU*c*U | | | UuCu*U*a | | | UCG | |
| 1205 | G*a*AuAcC | 2781 | 1109-1129 | (VPmU)*C*gAu | 1207 | 1107-1129 | AAGAAUACCA | 3919 |
| | aGgAuCaGu | | | AcUgAuCcUgGu | | | GGAUCAGUAU | |
| | AuC*g*A | | | AuUc*U*u | | | CGA | |
| 1206 | A*a*UaCcA | 2782 | 1110-1130 | (VPmA)*U*cGa | 1208 | 1108-1130 | AGAAUACCAG | 3920 |
| | gGaUcAgUa | | | UaCuGaUcCuGg | | | GAUCAGUAUC | |
| | UcG*a*U | | | UaUu*C*u | | | GAG | |
| 1207 | A*u*AcCaG | 2783 | 1111-1131 | (VPmU)*C*uCg | 1209 | 1109-1131 | GAAUACCAGG | 3921 |
| | gAuCaGuAu | | | AuAcUgAuCcUg | | | AUCAGUAUCG | |
| | CgA*g*A | | | GuAu*U*c | | | AGA | |
| 1208 | U*a*CcAgG | 2784 | 1112-1132 | (VPmA)*U*cUc | 1210 | 1110-1132 | AAUACCAGGA | 3922 |
| | aUcAgUaUc | | | GaUaCuGaUcCu | | | UCAGUAUCGA | |
| | GaG*a*U | | | GgUa*U*u | | | GAG | |
| 1209 | A*c*CaGgA | 2785 | 1113-1133 | (VPmU)*C*uCu | 1211 | 1111-1133 | AUACCAGGAU | 3923 |
| | uCaGuAuCg | | | CgAuAcUgAuCc | | | CAGUAUCGAG | |
| | AgA*g*A | | | UgGu*A*u | | | AGA | |
| 1210 | C*c*AgGaU | 2786 | 1114-1134 | (VPmA)*U*cUc | 1212 | 1112-1134 | UACCAGGAUC | 3924 |
| | cAgUaUcGa | | | UcGaUaCuGaUc | | | AGUAUCGAGA | |
| | GaG*a*U | | | CuGg*U*a | | | GAG | |
| 1211 | C*a*GgAuC | 2787 | 1115-1135 | (VPmA)*C*uCu | 1213 | 1113-1135 | ACCAGGAUCA | 3925 |
| | aGuAuCgAg | | | CuCgAuAcUgAu | | | GUAUCGAGAG | |
| | AgA*g*U | | | CcUg*G*u | | | AGG | |
| 1212 | A*g*GaUcA | 2788 | 1116-1136 | (VPmG)*C*cUc | 1214 | 1114-1136 | CCAGGAUCAG | 3926 |
| | gUaUcGaGa | | | UcUcGaUaCuGa | | | UAUCGAGAGA | |
| | GaG*g*C | | | UcCu*G*g | | | GGC | |
| 1213 | C*c*UgGcA | 2789 | 1143-1163 | (VPmG)*G*aAa | 1215 | 1141-1163 | GGCCUGGCAG | 3927 |
| | gAaGgGcGc | | | GcGcCcUuCuGc | | | AAGGGCGCUU | |
| | UuU*c*C | | | CaGg*C*c | | | UCC | |
| 1214 | A*g*AaGgG | 2790 | 1149-1169 | (VPmG)*U*gGg | 1216 | 1147-1169 | GCAGAAGGGC | 3928 |
| | cGcUuUcCa | | | UgGaAaGcGcCc | | | GCUUUCCACC | |
| | CcC*a*C | | | UuCu*G*c | | | CAC | |
| 1215 | G*a*AgGgC | 2791 | 1150-1170 | (VPmA)*G*uGg | 1217 | 1148-1170 | CAGAAGGGCG | 3929 |
| | gCuUuCcAc | | | GuGgAaAgCgCc | | | CUUUCCACCC | |
| | CcA*c*U | | | CuUc*U*g | | | ACU | |
| 1216 | C*u*GuUuA | 2792 | 1177-1197 | (VPmU)*C*uCg | 1218 | 1175-1197 | CCCUGUUUAG | 3930 |
| | gUcCaCcAc | | | GuGgUgGaCuAa | | | UCCACCACCG | |
| | CgA*g*A | | | AcAg*G*g | | | AGA | |
| 1217 | U*g*UuUaG | 2793 | 1178-1198 | (VPmG)*U*cUc | 1219 | 1176-1198 | CCUGUUUAGU | 3931 |
| | uCcAcCaCc | | | GgUgGuGgAcUa | | | CCACCACCGA | |
| | GaG*a*C | | | AaCa*G*g | | | GAC | |
| 1218 | G*u*UuAgU | 2794 | 1179-1199 | (VPmU)*G*uCu | 1220 | 1177-1199 | CUGUUUAGUC | 3932 |
| | cCaCcAcCg | | | CgGuGgUgGaCu | | | CACCACCGAG | |
| | AgA*c*A | | | AaAc*A*g | | | ACA | |
| 1219 | U*u*UaGuC | 2795 | 1180-1200 | (VPmA)*U*gUc | 1221 | 1178-1200 | UGUUUAGUCC | 3933 |
| | cAcCaCcGa | | | UcGgUgGuGgAc | | | ACCACCGAGA | |
| | GaC*a*U | | | UaAa*C*a | | | CAU | |
| 1220 | U*u*AgUcC | 2796 | 1181-1201 | (VPmG)*A*uGu | 1222 | 1179-1201 | GUUUAGUCCA | 3934 |
| | aCcAcCgAg | | | CuCgGuGgUgGa | | | CCACCGAGAC | |
| | AcA*u*C | | | CuAa*A*c | | | AUC | |
| 1221 | U*a*GuCcA | 2797 | 1182-1202 | (VPmU)*G*aUg | 1223 | 1180-1202 | UUUAGUCCAC | 3935 |
| | cCaCcGaGa | | | UcUcGgUgGuGg | | | CACCGAGACA | |
| | CaU*c*A | | | AcUa*A*a | | | UCA | |
| 1222 | A*g*UcCaC | 2798 | 1183-1203 | (VPmG)*U*gAu | 1224 | 1181-1203 | UUAGUCCACC | 3936 |
| | cAcCgAgAc | | | GuCuCgGuGgUg | | | ACCGAGACAU | |
| | AuC*a*C | | | GaCu*A*a | | | CAC | |
| 1223 | G*u*CcAcC | 2799 | 1184-1204 | (VPmA)*G*uGa | 1225 | 1182-1204 | UAGUCCACCA | 3937 |
| | aCcGaGaCa | | | UgUcUcGgUgGu | | | CCGAGACAUC | |
| | UcA*c*U | | | GgAc*U*a | | | ACU | |
| 1224 | C*c*AcCaC | 2800 | 1186-1206 | (VPmA)*A*aGu | 1226 | 1184-1206 | GUCCACCACC | 3938 |
| | cGaGaCaUc | | | GaUgUcUcGgUg | | | GAGACAUCAC | |
| | AcU*u*U | | | GuGg*A*c | | | UUG | |
| 1225 | C*a*CcAcC | 2801 | 1187-1207 | (VPmA)*C*aAg | 1227 | 1185-1207 | UCCACCACCG | 3939 |
| | gAgAcAuCa | | | UgAuGuCuCgGu | | | AGACAUCACU | |
| | CuU*g*U | | | GgUg*G*a | | | UGG | |
| 1226 | A*c*CaCcG | 2802 | 1188-1208 | (VPmU)*C*cAa | 1228 | 1186-1208 | CCACCACCGA | 3940 |
| | aGaCaUcAc | | | GuGaUgUcUcGg | | | GACAUCACUU | |
| | UuG*g*A | | | UgGu*G*g | | | GGA | |
| 1227 | C*c*AcCgA | 2803 | 1189-1209 | (VPmG)*U*cCa | 1229 | 1187-1209 | CACCACCGAG | 3941 |
| | gAcAuCaCu | | | AgUgAuGuCuCg | | | ACAUCACUUG | |
| | UgG*a*C | | | GuGg*U*g | | | GAC | |
| 1228 | U*g*CuGcG | 2804 | 1283-1303 | (VPmA)*C*aUu | 1230 | 1281-1303 | GGUGCUGCGG | 3942 |
| | gUuGaAuCc | | | GgAuUcAaCcGc | | | UUGAAUCCAA | |
| | AaU*g*U | | | AgCa*C*c | | | UGG | |
| 1229 | A*g*CcUcC | 2805 | 1310-1330 | (VPmA)*G*aAa | 1231 | 1308-1330 | GGAGCCUCCC | 3943 |
| | cGcCaUgGa | | | UcCaUgGcGgGa | | | GCCAUGGAUU | |
| | UuU*c*U | | | GgCu*C*c | | | UCU | |
| 1230 | G*c*CuCcC | 2806 | 1311-1331 | (VPmG)*A*gAa | 1232 | 1309-1331 | GAGCCUCCCG | 3944 |
| | gCcAuGgAu | | | AuCcAuGgCgGg | | | CCAUGGAUUU | |
| | UuC*u*C | | | AgGc*U*c | | | CUC | |
| 1231 | C*u*CcCgC | 2807 | 1313-1333 | (VPmU)*A*gAg | 1233 | 1311-1333 | GCCUCCCGCC | 3945 |
| | cAuGgAuUu | | | AaAuCcAuGgCg | | | AUGGAUUUCU | |
| | CuC*u*A | | | GgAg*G*c | | | CUA | |
| 1232 | U*c*CcGcC | 2808 | 1314-1334 | (VPmA)*U*aGa | 1234 | 1312-1334 | CCUCCCGCCA | 3946 |
| | aUgGaUuUc | | | GaAaUcCaUgGc | | | UGGAUUUCUC | |
| | UcU*a*U | | | GgGa*G*g | | | UAG | |
| 1233 | C*c*CgCcA | 2809 | 1315-1335 | (VPmA)*C*uAg | 1235 | 1313-1335 | CUCCCGCCAU | 3947 |
| | uGgAuUuCu | | | AgAaAuCcAuGg | | | GGAUUUCUCU | |
| | CuA*g*U | | | CgGg*A*g | | | AGG | |
| 1234 | C*g*CcAuG | 2810 | 1317-1337 | (VPmA)*U*cCu | 1236 | 1315-1337 | CCCGCCAUGG | 3948 |
| | gAuUuCuCu | | | AgAgAaAuCcAu | | | AUUUCUCUAG | |
| | AgG*a*U | | | GgCg*G*g | | | GAG | |
| 1235 | U*a*GgAgA | 2811 | 1332-1352 | (VPmG)*C*cAg | 1237 | 1330-1352 | UCUAGGAGAC | 3949 |
| | cUuAgAgAg | | | CuCuCuAaGuCu | | | UUAGAGAGCU | |
| | CuG*g*C | | | CcUa*G*a | | | GGC | |
| 1236 | A*g*GaGaC | 2812 | 1333-1353 | (VPmU)*G*cCa | 1238 | 1331-1353 | CUAGGAGACU | 3950 |
| | uUaGaGaGc | | | GcUcUcUaAgUc | | | UAGAGAGCUG | |
| | UgG*c*A | | | UcCu*A*g | | | GCA | |
| 1237 | G*g*AaCaC | 2813 | 1334-1354 | (VPmG)*C*gGu | 1239 | 1332-1354 | AGGGAACACG | 3951 |
| | gUcUaGcCc | | | GgGcUaGaCgUg | | | UCUAGCCCAC | |
| | AcC*g*C | | | UuCc*C*u | | | CGC | |
| 1238 | C*c*UaUgC | 2814 | 1396-1416 | (VPmU)*U*gCa | 1240 | 1394-1416 | GCCCUAUGCA | 3952 |
| | aAaGgUuAc | | | GuAaCcUuUgCa | | | AAGGUUACUG | |
| | UgC*a*A | | | UaGg*G*c | | | CAA | |
| 1239 | C*u*AuGcA | 2815 | 1397-1417 | (VPmG)*U*uGc | 1241 | 1395-1417 | CCCUAUGCAA | 3953 |
| | aAgGuUaCu | | | AgUaAcCuUuGc | | | AGGUUACUGC | |
| | GcA*a*C | | | AuAg*G*g | | | AAC | |
| 1240 | U*a*UgCaA | 2816 | 1398-1418 | (VPmG)*G*uUg | 1242 | 1396-1418 | UAUGCAAAGG | 3954 |
| | aGgUuAcUg | | | CaGuAaCcUuUg | | | UUACUGCAAC | |
| | CaA*c*C | | | CaUa*G*g | | | C | |
| 1241 | U*g*CaAaG | 2817 | 1400-1420 | (VPmA)*U*gGu | 1243 | 1398-1420 | UAUGCAAAGG | 3955 |
| | gUuAcUgCa | | | UgCaGuAaCcUu | | | UUACUGCAAC | |
| | AcC*a*U | | | UgCa*U*a | | | CAU | |
| 1242 | G*c*AaAgG | 2818 | 1411-1421 | (VPmA)*A*uGg | 1244 | 1399-1421 | AUGCAAAGGU | 3956 |
| | uUaCuGcAa | | | UuGcAgUaAcCu | | | UACUGCAACC | |
| | CcA*u*U | | | UuGc*A*u | | | AUU | |
| 1243 | C*a*AaGgU | 2819 | 1402-1422 | (VPmG)*A*aUg | 1245 | 1400-1422 | UGCAAAGGUU | 3957 |
| | uAcUgCaAc | | | GuUgCaGuAaCc | | | ACUGCAACCA | |
| | CaU*u*C | | | UuUg*C*a | | | UUC | |
| 1244 | A*a*AgGuU | 2820 | 1403-1423 | (VPmG)*G*aAu | 1246 | 1401-1423 | GCAAAGGUUA | 3958 |
| | aCuGcAaCc | | | GgUuGcAgUaAc | | | CUGCAACCAU | |
| | AuU*c*C | | | CuUu*G*c | | | UCC | |
| 1245 | A*a*GgUuA | 2821 | 1404-1424 | (VPmU)*G*gAa | 1247 | 1402-1424 | CAAAGGUUAC | 3959 |
| | cUgCaAcCa | | | UgGuUgCaGuAa | | | UGCAACCAUU | |
| | UuC*c*A | | | CcUu*U*g | | | CCA | |
| 1246 | A*g*GuUaC | 2822 | 1405-1425 | (VPmA)*U*gGa | 1248 | 1403-1425 | AAAGGUUACU | 3960 |
| | uGcAaCcAu | | | AuGgUuGcAgUa | | | GCAACCAUUC | |
| | UcC*a*U | | | AcCu*U*u | | | CAG | |
| 1247 | G*g*UuAcU | 2823 | 1406-1426 | (VPmG)*C*uGg | 1249 | 1404-1426 | AAGGUUACUG | 3961 |
| | gCaAcCaUu | | | AaUgGuUgCaGu | | | CAACCAUUCC | |
| | CcA*g*C | | | AaCc*U*u | | | AGC | |
| 1248 | U*a*CuGcA | 2824 | 1409-1429 | (VPmA)*U*gGc | 1250 | 1407-1429 | GUUACUGCAA | 3962 |
| | aCcAuUcCa | | | UgGaAuGgUuGc | | | CCAUUCCAGC | |
| | GcC*a*U | | | AgUa*A*c | | | CA | |
| 1249 | C*g*GuCaA | 2825 | 1502-1522 | (VPmA)*G*cAu | 1251 | 1500-1522 | CCCGGUCAAG | 3963 |
| | gUcCaAgUc | | | GaCuUgGaCuUg | | | UCCAAGUCAU | |
| | AuG*c*U | | | AcCg*G*g | | | GC | |
| 1250 | G*g*UcAaG | 2826 | 1503-1523 | (VPmU)*C*gCa | 1252 | 1501-1523 | CCGGUCAAGU | 3964 |
| | uCcAaGuCa | | | UgAcUuGgAcUu | | | CCAAGUCAUG | |
| | UgC*g*A | | | GaCc*G*g | | | CGA | |
| 1251 | G*u*CaAgU | 2827 | 1504-1524 | (VPmA)*U*cGc | 1253 | 1502-1524 | CGGUCAAGUC | 3965 |
| | cCaAgUcAu | | | AuGaCuUgGaCu | | | CAAGUCAUGC | |
| | GcG*a*U | | | UgAc*C*g | | | GAG | |
| 1252 | U*c*AaGuC | 2828 | 1505-1525 | (VPmA)*C*uCg | 1254 | 1503-1525 | GGUCAAGUCC | 3966 |
| | cAaGuCaUg | | | CaUgAcUuGgAc | | | AAGUCAUGCG | |
| | CgA*g*U | | | UuGa*C*c | | | AGU | |
| 1253 | C*a*AgUcC | 2829 | 1506-1526 | (VPmA)*A*cUc | 1255 | 1504-1526 | GUCAAGUCCA | 3967 |
| | aAgUcAuGc | | | GcAuGaCuUgGa | | | AGUCAUGCGA | |
| | GaG*u*U | | | CuUg*A*c | | | GUU | |
| 1254 | A*a*GuCcA | 2830 | 1507-1527 | (VPmG)*A*aCu | 1256 | 1505-1527 | UCAAGUCCAA | 3968 |
| | aGuCaUgCg | | | CgCaUgAcUuGg | | | GUCAUGCGAG | |
| | AgU*u*C | | | AcUu*G*a | | | UUC | |
| 1255 | A*g*UcCaA | 2831 | 1508-1528 | (VPmA)*G*aAc | 1257 | 1506-1528 | CAAGUCCAAG | 3969 |
| | gUcAuGcGa | | | UcGcAuGaCuUg | | | UCAUGCGAGU | |
| | GuU*c*U | | | GaCu*U*g | | | UCU | |
| 1256 | G*u*CcAaG | 2832 | 1509-1529 | (VPmA)*A*gAa | 1258 | 1507-1529 | AAGUCCAAGU | 3970 |
| | uCaUgCgAg | | | CuCgCaUgAcUu | | | CAUGCGAGUU | |
| | UuC*u*U | | | GgAc*U*u | | | CUG | |
| 1257 | U*c*CaAgU | 2833 | 1510-1530 | (VPmG)*C*aGa | 1259 | 1508-1530 | AGUCCAAGUC | 3971 |
| | cAuGcGaGu | | | AcUcGcAuGaCu | | | AUGCGAGUUC | |
| | UcU*g*C | | | UgGa*C*u | | | UGC | |
| 1258 | C*c*AaGuC | 2834 | 1511-1531 | (VPmA)*G*cAg | 1260 | 1509-1531 | GUCCAAGUCA | 3972 |
| | aUgCgAgUu | | | AaCuCgCaUgAc | | | UGCGAGUUCU | |
| | CuG*c*U | | | UuGg*A*c | | | GCG | |
| 1259 | C*a*AgUcA | 2835 | 1512-1532 | (VPmA)*C*gCa | 1261 | 1510-1532 | UCCAAGUCAU | 3973 |
| | uGcGaGuUc | | | GaAcUcGcAuGa | | | GCGAGUUCUG | |
| | UgC*g*U | | | CuUg*G*a | | | CGG | |
| 1260 | A*g*UcAuG | 2836 | 1514-1534 | (VPmU)*G*cCg | 1262 | 1512-1534 | CAAGUCAUGC | 3974 |
| | cGaGuUcUg | | | CaGaAcUcGcAu | | | GAGUUCUGCG | |
| | CgG*c*A | | | GaCu*U*g | | | GCA | |
| 1261 | G*u*CaUgC | 2837 | 1515-1535 | (VPmU)*U*gCc | 1263 | 1513-1535 | AAGUCAUGCG | 3975 |
| | gAgUuCuGc | | | GcAgAaCuCgCa | | | AGUUCUGCGG | |
| | GgC*a*A | | | UgAc*U*u | | | CAA | |
| 1262 | U*c*AuGcG | 2838 | 1516-1536 | (VPmA)*U*uGc | 1264 | 1514-1536 | AGUCAUGCGA | 3976 |
| | aGuUcUgCg | | | CgCaGaAcUcGc | | | GUUCUGCGGC | |
| | GcA*a*U | | | AuGa*C*u | | | AAG | |
| 1263 | G*c*GaGuU | 2839 | 1520-1540 | (VPmA)*C*gUc | 1265 | 1518-1540 | AUGCGAGUUC | 3977 |
| | cUgCgGcAa | | | UuGcCgCaGaAc | | | UGCGGCAAGA | |
| | GaC*g*U | | | UcGc*A*u | | | CGU | |
| 1264 | C*g*AgUuC | 2840 | 1521-1541 | (VPmA)*A*cGu | 1266 | 1519-1541 | UGCGAGUUCU | 3978 |
| | uGcGgCaAg | | | CuUgCcGcAgAa | | | GCGGCAAGAC | |
| | AcG*u*U | | | CuCg*C*a | | | GUU | |
| 1265 | G*a*GuUcU | 2841 | 1522-1542 | (VPmG)*A*aCg | 1267 | 1520-1542 | GCGAGUUCUG | 3979 |
| | gCgGcAaGa | | | UcUuGcCgCaGa | | | CGGCAAGACG | |
| | CgU*u*C | | | AcUc*G*c | | | UUC | |
| 1266 | A*g*UuCuG | 2842 | 1523-1543 | (VPmU)*G*aAc | 1268 | 1521-1543 | CGAGUUCUGC | 3980 |
| | cGgCaAgAc | | | GuCuUgCcGcAg | | | GGCAAGACGU | |
| | GuU*c*A | | | AaCu*C*g | | | UCA | |
| 1267 | G*u*UcUgC | 2843 | 1524-1544 | (VPmU)*U*gAa | 1269 | 1522-1544 | GAGUUCUGCG | 3981 |
| | gGcAaGaCg | | | CgUcUuGcCgCa | | | GCAAGACGUU | |
| | UuC*a*A | | | GaAc*U*c | | | CAA | |
| 1268 | U*u*CuGcG | 2844 | 1525-1545 | (VPmU)*U*uGa | 1270 | 1523-1545 | AGUUCUGCGG | 3982 |
| | gCaAgAcGu | | | AcGuCuUgCcGc | | | CAAGACGUUC | |
| | UcA*a*A | | | AgAa*C*u | | | AAA | |
| 1269 | U*c*UgCgG | 2845 | 1526-1546 | (VPmA)*U*uUg | 1271 | 1524-1546 | GUUCUGCGGC | 3983 |
| | cAaGaCgUu | | | AaCgUcUuGcCg | | | AAGACGUUCA | |
| | CaA*a*U | | | CaGa*A*c | | | AAU | |
| 1270 | C*u*GcGgC | 2846 | 1527-1547 | (VPmA)*A*uUu | 1272 | 1525-1547 | UUCUGCGGCA | 3984 |
| | aAgAcGuUc | | | GaAcGuCuUgCc | | | AGACGUUCAA | |
| | AaA*u*U | | | GcAg*A*a | | | AUU | |
| 1271 | U*g*CgGcA | 2847 | 1528-1548 | (VPmA)*A*aUu | 1273 | 1526-1548 | UCUGCGGCAA | 3985 |
| | aGaCgUuCa | | | UgAaCgUcUuGc | | | GACGUUCAAA | |
| | AaU*u*U | | | CgCa*G*a | | | UUU | |
| 1272 | G*c*GgCaA | 2848 | 1529-1549 | (VPmG)*A*aAu | 1274 | 1527-1549 | CUGCGGCAAG | 3986 |
| | gAcGuUcAa | | | UuGaAcGuCuUg | | | ACGUUCAAAU | |
| | AuU*u*C | | | CcGc*A*g | | | UUC | |
| 1273 | C*a*UgAcG | 2849 | 1683-1703 | (VPmU)*C*gUc | 1275 | 1681-1703 | CCCAUGACGG | 3987 |
| | gUcAaGuCc | | | GgAcUuGaCcGu | | | UCAAGUCCGA | |
| | GaC*g*A | | | CaUg*G*g | | | CGA | |
| 1274 | C*a*AgUcC | 2850 | 1773-1793 | (VPmA)*A*cUu | 1276 | 1771-1793 | CUCAAGUCCG | 3988 |
| | gUgGuGgCc | | | GgCcAcCaCgGa | | | UGGUGGCCAA | |
| | AaG*u*U | | | CuUg*A*g | | | GUU | |
| 1275 | A*a*GuCcG | 2851 | 1774-1794 | (VPmG)*A*aCu | 1277 | 1772-1794 | UCAAGUCCGU | 3989 |
| | uGgUgGcCa | | | UgGcCaCcAcGg | | | GGUGGCCAAG | |
| | AgU*u*C | | | AcUu*G*a | | | UUC | |
| 1276 | U*c*CgUgG | 2852 | 1777-1797 | (VPmA)*U*uGa | 1278 | 1775-1797 | AGUCCGUGGU | 3990 |
| | uGgCcAaGu | | | AcUuGgCcAcCa | | | GGCCAAGUUC | |
| | UcA*a*U | | | CgGa*C*u | | | AAG | |
| 1277 | A*g*AgGaG | 2853 | 1845-1865 | (VPmU)*C*uUc | 1279 | 1843-1865 | GAAGAGGAGG | 3991 |
| | gAcGaCgAg | | | CuCgUcGuCcUc | | | ACGACGAGGA | |
| | GaA*g*A | | | CuCu*U*c | | | AGA | |
| 1278 | G*a*GgAgG | 2854 | 1846-1866 | (VPmA)*U*cUu | 1280 | 1844-1866 | AAGAGGAGGA | 3992 |
| | aCgAcGaGg | | | CcUcGuCgUcCu | | | CGACGAGGAA | |
| | AaG*a*U | | | CcUc*U*u | | | GAG | |
| 1279 | G*g*AgCuG | 2855 | 1893-1913 | (VPmA)*U*cUc | 1281 | 1891-1913 | GAGGAGCUGA | 3993 |
| | aCgGaGaGc | | | GcUcUcCgUcAg | | | CGGAGAGCGA | |
| | GaG*a*U | | | CuCc*U*c | | | GAG | |
| 1280 | A*g*CuGaC | 2856 | 1895-1915 | (VPmA)*C*cUc | 1282 | 1893-1915 | GGAGCUGACG | 3994 |
| | gGaGaGcGa | | | UcGcUcUcCgUc | | | GAGAGCGAGA | |
| | GaG*g*U | | | AgCu*C*c | | | GGG | |
| 1281 | G*c*UgAcG | 2857 | 1896-1916 | (VPmA)*C*cCu | 1283 | 1894-1916 | GAGCUGACGG | 3995 |
| | gAgAgCgAg | | | CuCgCuCuCcGu | | | AGAGCGAGAG | |
| | AgG*g*U | | | CaGc*U*c | | | GGU | |
| 1282 | C*u*GaCgG | 2858 | 1897-1917 | (VPmA)*A*cCc | 1284 | 1895-1917 | AGCUGACGGA | 3996 |
| | aGaGcGaGa | | | UcUcGcUcUcCg | | | GAGCGAGAGG | |
| | GgG*u*U | | | UcAg*C*u | | | GUG | |
| 1283 | C*a*UgCaG | 2859 | 2046-2066 | (VPmG)*C*cUc | 1285 | 2044-2066 | UCCAUGCAGC | 3997 |
| | cAcUuCaGc | | | GcUgAaGuGcUg | | | ACUUCAGCGA | |
| | GaG*g*C | | | CaUg*G*a | | | GGC | |
| 1284 | U*g*CaGcA | 2860 | 2048-2068 | (VPmA)*G*gCc | 1286 | 2046-2068 | CAUGCAGCAC | 3998 |
| | cUuCaGcGa | | | UcGcUgAaGuGc | | | UUCAGCGAGG | |
| | GgC*c*U | | | UgCa*U*g | | | CCU | |
| 1285 | U*c*AgCgA | 2861 | 2057-2077 | (VPmA)*C*uGg | 1287 | 2055-2077 | CUUCAGCGAG | 3999 |
| | gGcCuUcCa | | | UgGaAgGcCuCg | | | GCCUUCCACC | |
| | CcA*g*U | | | CuGa*A*g | | | AGG | |
| 1286 | A*c*CgCaU | 2862 | 2168-2188 | (VPmA)*A*gUg | 1288 | 2166-2188 | GGACCGCAUA | 4000 |
| | aGaCgAuGg | | | CcAuCgUcUaUg | | | GACGAUGGCA | |
| | CaC*u*U | | | CgGu*C*c | | | CU | |
| 1287 | C*g*CaUaG | 2863 | 2170-2190 | (VPmA)*A*cAg | 1289 | 2168-2190 | ACCGCAUAGA | 4001 |
| | aCgAuGgCa | | | UgCcAuCgUcUa | | | CGAUGGCACU | |
| | CuG*u*U | | | UgCg*G*u | | | GUU | |
| 1288 | C*a*UaGaC | 2864 | 2172-2192 | (VPmU)*U*aAc | 1290 | 2170-2192 | CGCAUAGACG | 4002 |
| | gAuGgCaCu | | | AgUgCcAuCgUc | | | AUGGCACUGU | |
| | GuU*a*A | | | UaUg*C*g | | | UAA | |
| 1289 | A*g*GcAgC | 2865 | 2383-2403 | (VPmG)*A*aGg | 1291 | 2381-2403 | CCAGGCAGCU | 4003 |
| | uCaAaGaUc | | | GaUcUuUgAgCu | | | CAAAGAUCCC | |
| | CcU*u*C | | | GcCu*G*g | | | UUC | |
| 1290 | G*g*CaGcU | 2866 | 2384-2404 | (VPmG)*G*aAg | 1292 | 2382-2404 | CAGGCAGCUC | 4004 |
| | cAaAgAuCc | | | GgAuCuUuGaGc | | | AAAGAUCCCU | |
| | CuU*c*C | | | UgCc*U*g | | | UCC | |
| 1291 | G*c*AgCuC | 2867 | 2385-2405 | (VPmA)*G*gAa | 1293 | 2383-2405 | AGGCAGCUCA | 3562 |
| | aAaGaUcCc | | | GgGaUcUuUgAg | | | AAGAUCCCUU | |
| | UuC*c*U | | | CuGc*C*u | | | CCU | |
| 1292 | U*c*GgAgA | 2868 | 2411-2431 | (VPmG)*C*gAu | 1294 | 2409-2431 | CUUCGGAGAC | 4005 |
| | cUcCaGaCa | | | UgUcUgGaGuCu | | | UCCAGACAAU | |
| | AuC*g*C | | | CcGa*A*g | | | CGC | |
| 1293 | C*g*GaGaC | 2869 | 2412-2432 | (VPmG)*G*cGa | 1295 | 2410-2432 | UUCGGAGACU | 4006 |
| | uCcAgAcAa | | | UuGuCuGgAgUc | | | CCAGACAAUC | |
| | UcG*c*C | | | UcCg*A*a | | | GCC | |
| 1294 | G*g*AgAcU | 2870 | 2413-2433 | (VPmA)*G*gCg | 1296 | 2411-2433 | UCGGAGACUC | 4007 |
| | cCaGaCaAu | | | AuUgUcUgGaGu | | | CAGACAAUCG | |
| | CgC*c*U | | | CuCc*G*a | | | CCU | |
| 1295 | G*a*GaCuC | 2871 | 2414-2434 | (VPmA)*A*gGc | 1297 | 2412-2434 | CGGAGACUCC | 4008 |
| | cAgAcAaUc | | | GaUuGuCuGgAg | | | AGACAAUCGC | |
| | GcC*u*U | | | UcUc*C*g | | | CUU | |
| 1296 | A*g*AcUcC | 2872 | 2415-2435 | (VPmA)*A*aGg | 1298 | 2413-2435 | GGAGACUCCA | 4009 |
| | aGaCaAuCg | | | CgAuUgUcUgGa | | | GACAAUCGCC | |
| | CcU*u*U | | | GuCu*C*c | | | UUU | |
| 1297 | G*a*CuCcA | 2873 | 2416-2436 | (VPmA)*A*aAg | 1299 | 2414-2436 | GAGACUCCAG | 4010 |
| | gAcAaUcGc | | | GcGaUuGuCuGg | | | ACAAUCGCCU | |
| | CuU*u*U | | | AgUc*U*c | | | UUU | |
| 1298 | A*c*UcCaG | 2874 | 2417-2437 | (VPmA)*A*aAa | 1300 | 2415-2437 | AGACUCCAGA | 4011 |
| | aCaAuCgCc | | | GgCgAuUgUcUg | | | CAAUCGCCUU | |
| | UuU*u*U | | | GaGu*C*u | | | UUG | |
| 1299 | C*u*CcAgA | 2875 | 2418-2438 | (VPmG)*C*aAa | 1301 | 2416-2438 | GACUCCAGAC | 4012 |
| | cAaUcGcCu | | | AgGcGaUuGuCu | | | AAUCGCCUUU | |
| | UuU*g*C | | | GgAg*U*c | | | UGC | |
| 1300 | C*c*AgAcA | 2876 | 2420-2440 | (VPmA)*G*gCa | 1302 | 2418-2440 | CUCCAGACAA | 4013 |
| | aUcGcCuUu | | | AaAgGcGaUuGu | | | UCGCCUUUUG | |
| | UgC*c*U | | | CuGg*A*g | | | CCU | |
| 1301 | C*a*GaCaA | 2877 | 2421-2441 | (VPmG)*A*gGc | 1303 | 2419-2441 | UCCAGACAAU | 4014 |
| | uCgCcUuUu | | | AaAaGgCgAuUg | | | CGCCUUUUGC | |
| | GcC*u*C | | | UcUg*G*a | | | CUC | |
| 1302 | G*a*CgCaG | 2878 | 2600-2620 | (VPmA)*C*uCa | 1304 | 2598-2620 | CAGACGCAGC | 4015 |
| | cGaCaCuUg | | | CaAgUgUcGcUg | | | GACACUUGUG | |
| | UgA*g*U | | | CgUc*U*g | | | AGU | |
| 1303 | C*g*CaGcG | 2879 | 2602-2622 | (VPmG)*U*aCu | 1305 | 2600-2622 | GACGCAGCGA | 4016 |
| | aCaCuUgUg | | | CaCaAgUgUcGc | | | CACUUGUGAG | |
| | AgU*a*C | | | UgCg*U*c | | | UAC | |
| 1304 | G*c*AgCgA | 2880 | 2603-2623 | (VPmA)*G*uAc | 1306 | 2601-2623 | ACGCAGCGAC | 4017 |
| | cAcUuGuGa | | | UcAcAaGuGuCg | | | ACUUGUGAGU | |
| | GuA*c*U | | | CuGc*G*u | | | ACU | |
| 1305 | C*a*GcGaC | 2881 | 2604-2624 | (VPmA)*A*gUa | 1307 | 2602-2624 | CGCAGCGACA | 4018 |
| | aCuUgUgAg | | | CuCaCaAgUgUc | | | CUUGUGAGUA | |
| | UaC*u*U | | | GcUg*C*g | | | CUG | |
| 1306 | G*a*CaCuU | 2882 | 2608-2628 | (VPmA)*C*cAc | 1308 | 2606-2628 | GCGACACUUG | 4019 |
| | gUgAgUaCu | | | AgUaCuCaCaAg | | | UGAGUACUGU | |
| | GuG*g*U | | | UgUc*G*c | | | GGG | |
| 1307 | A*c*AcUuG | 2883 | 2609-2629 | (VPmU)*C*cCa | 1309 | 2607-2629 | CGACACUUGU | 4020 |
| | uGaGuAcUg | | | CaGuAcUcAcAa | | | GAGUACUGUG | |
| | UgG*g*A | | | GuGu*C*g | | | GGA | |
| 1308 | C*a*CuUgU | 2884 | 2610-2630 | (VPmU)*U*cCc | 1310 | 2608-2630 | GACACUUGUG | 4021 |
| | gAgUaCuGu | | | AcAgUaCuCaCa | | | AGUACUGUGG | |
| | GgG*a*A | | | AgUg*U*c | | | GAA | |
| 1309 | A*c*UuGuG | 2885 | 2611-2631 | (VPmU)*U*uCc | 1311 | 2609-2631 | ACACUUGUGA | 4022 |
| | aGuAcUgUg | | | CaCaGuAcUcAc | | | GUACUGUGGG | |
| | GgA*a*A | | | AaGu*G*u | | | AA | |
| 1310 | C*u*UgUgA | 2886 | 2612-2632 | (VPmA)*U*uUc | 1312 | 2610-2632 | CACUUGUGAG | 4023 |
| | gUaCuGuGg | | | CcAcAgUaCuCa | | | UACUGUGGGA | |
| | GaA*a*U | | | CaAg*U*g | | | AAG | |
| 1311 | U*u*GuGaG | 2887 | 2613-2633 | (VPmA)*C*uUu | 1313 | 2611-2633 | ACUUGUGAGU | 4024 |
| | uAcUgUgGg | | | CcCaCaGuAcUc | | | ACUGUGGGAA | |
| | AaA*g*U | | | AcAa*G*u | | | AGU | |
| 1312 | U*g*UgAgU | 2888 | 2614-2634 | (VPmG)*A*cUu | 1314 | 2612-2634 | CUUGUGAGUA | 4025 |
| | aCuGuGgGa | | | UcCcAcAgUaCu | | | CUGUGGGAAA | |
| | AaG*u*C | | | CaCa*A*g | | | GUC | |
| 1313 | G*u*GaGuA | 2889 | 2615-2635 | (VPmA)*G*aCu | 1315 | 2613-2635 | UUGUGAGUAC | 4026 |
| | cUgUgGgAa | | | UuCcCaCaGuAc | | | UGUGGGAAAG | |
| | AgU*c*U | | | UcAc*A*a | | | UCU | |
| 1314 | G*u*CuUcA | 2890 | 2632-2652 | (VPmA)*U*uGc | 1316 | 2630-2652 | AAGUCUUCAA | 4027 |
| | aGaAcUgUa | | | UaCaGuUcUuGa | | | GAACUGUAGC | |
| | GcA*a*U | | | AgAc*U*u | | | AAU | |
| 1315 | U*c*UuCaA | 2891 | 2633-2653 | (VPmG)*A*uUg | 1317 | 2631-2653 | AGUCUUCAAG | 4028 |
| | gAaCuGuAg | | | CuAcAgUuCuUg | | | AACUGUAGCA | |
| | CaA*u*C | | | AaGa*C*u | | | AUC | |
| 1316 | C*u*UcAaG | 2892 | 2634-2654 | (VPmA)*G*aUu | 1318 | 2632-2654 | GUCUUCAAGA | 4029 |
| | aAcUgUaGc | | | GcUaCaGuUcUu | | | ACUGUAGCAA | |
| | AaU*c*U | | | GaAg*A*c | | | UCU | |
| 1317 | U*u*CaAgA | 2893 | 2635-2655 | (VPmG)*A*gAu | 1319 | 2633-2655 | UCUUCAAGAA | 4030 |
| | aCuGuAgCa | | | UgCuAcAgUuCu | | | CUGUAGCAAU | |
| | AuC*u*C | | | UgAa*G*a | | | CUC | |
| 1318 | U*c*AaGaA | 2894 | 2636-2656 | (VPmU)*G*aGa | 1320 | 2634-2656 | CUUCAAGAAC | 4031 |
| | cUgUaGcAa | | | UuGcUaCaGuUc | | | UGUAGCAAUC | |
| | UCU*c*A | | | UuGa*A*g | | | UCA | |
| 1319 | C*a*AgAaC | 2895 | 2637-2657 | (VPmG)*U*gAg | 1321 | 2635-2657 | UUCAAGAACU | 4032 |
| | uGuAgCaAu | | | AuUgCuAcAgUu | | | GUAGCAAUCU | |
| | CuC*a*C | | | CuUg*A*a | | | CAC | |
| 1320 | A*a*GaAcU | 2896 | 2638-2658 | (VPmA)*G*uGa | 1322 | 2636-2658 | UCAAGAACUG | 4033 |
| | gUaGcAaUc | | | GaUuGcUaCaGu | | | UAGCAAUCUC | |
| | UcA*c*U | | | UcUu*G*a | | | ACU | |
| 1321 | G*a*GaAgC | 2897 | 2667-2687 | (VPmA)*U*uUc | 1323 | 2665-2687 | AGGAGAAGCC | 4034 |
| | CAcAcGgGc | | | GcCcGuGuGgCu | | | ACACGGGCGA | |
| | GaA*a*U | | | UcUc*C*u | | | AAG | |
| 1322 | A*g*CcAcA | 2898 | 2671-2691 | (VPmA)*G*gCc | 1324 | 2669-2691 | GAAGCCACAC | 4035 |
| | cGgGcGaAa | | | UuUcGcCcGuGu | | | GGGCGAAAGG | |
| | GgC*c*U | | | GgCu*U*c | | | CCU | |
| 1323 | G*c*CaCaC | 2899 | 2672-2692 | (VPmA)*A*gGc | 1325 | 2670-2692 | AAGCCACACG | 4036 |
| | gGgCgAaAg | | | CuUuCgCcCgUg | | | GGCGAAAGGC | |
| | GcC*u*U | | | UgGc*U*u | | | CUU | |
| 1324 | A*c*AcGgG | 2900 | 2674-2695 | (VPmU)*A*uAa | 1326 | 2673-2695 | CCACACGGGC | 4037 |
| | cGaAaGgCc | | | GgCcUuUcGcCc | | | GAAAGGCCUU | |
| | UuA*u*A | | | GuGu*G*g | | | AUA | |
| 1325 | C*a*CgGgC | 2901 | 2676-2696 | (VPmU)*U*aUa | 1327 | 2674-2696 | CACACGGGCG | 4038 |
| | gAaAgGcCu | | | AgGcCuUuCgCc | | | AAAGGCCUUA | |
| | UaU*a*A | | | CgUg*U*g | | | UAA | |
| 1326 | A*c*GgGcG | 2902 | 2677-2697 | (VPmU)*U*uAu | 1328 | 2675-2697 | ACACGGGCGA | 4039 |
| | aAaGgCcUu | | | AaGgCcUuUcGc | | | AAGGCCUUAU | |
| | AuA*a*A | | | CcGu*G*u | | | AAA | |
| 1327 | C*g*GgCgA | 2903 | 2678-2698 | (VPmA)*U*uUa | 1329 | 2676-2698 | CACGGGCGAA | 4040 |
| | aAgGcCuUa | | | UaAgGcCuUuCg | | | AGGCCUUAUA | |
| | UaA*a*U | | | CcCg*U*g | | | AAU | |
| 1328 | G*g*GcGaA | 2904 | 2679-2699 | (VPmA)*A*uUu | 1330 | 2677-2699 | ACGGGCGAAA | 4041 |
| | aGgCcUuAu | | | AuAaGgCcUuUc | | | GGCCUUAUAA | |
| | AaA*u*U | | | GcCc*G*u | | | AUG | |
| 1329 | G*g*CgAaA | 2905 | 2680-2700 | (VPmG)*C*aUu | 1331 | 2678-2700 | CGGGCGAAAG | 4042 |
| | gGcCuUaUa | | | UaUaAgGcCuUu | | | GCCUUAUAAA | |
| | AaU*g*C | | | CgCc*C*g | | | UGC | |
| 1330 | G*c*GaAaG | 2906 | 2681-2701 | (VPmA)*G*cAu | 1332 | 2679-2701 | GGGCGAAAGG | 4043 |
| | gCcUuAuAa | | | UuAuAaGgCcUu | | | CCUUAUAAAU | |
| | AuG*c*U | | | UcGc*C*c | | | GCG | |
| 1331 | G*a*AaGgC | 2907 | 2683-2703 | (VPmA)*U*cGc | 1333 | 2681-2703 | GCGAAAGGCC | 4044 |
| | cUuAuAaAu | | | AuUuAuAaGgCc | | | UUAUAAAUGC | |
| | GcG*a*U | | | UuUc*G*C | | | GAG | |
| 1332 | A*a*AgGcC | 2908 | 2684-2704 | (VPmG)*C*uCg | 1334 | 2682-2704 | CGAAAGGCCU | 4045 |
| | uUaUaAaUg | | | CaUuUaUaAgGc | | | UAUAAAUGCG | |
| | CgA*g*C | | | CuUu*C*g | | | AGG | |
| 1333 | A*a*GgCcU | 2909 | 2685-2705 | (VPmA)*G*cUc | 1335 | 2683-2705 | GAAAGGCCUU | 4046 |
| | uAuAaAuGc | | | GcAuUuAuAaGg | | | AUAAAUGCGA | |
| | GaG*c*U | | | CcUu*U*c | | | GCU | |
| 1334 | A*g*GcCuU | 2910 | 2686-2706 | (VPmA)*A*gCu | 1336 | 2684-2706 | AAAGGCCUUA | 4047 |
| | aUaAaUgCg | | | CgCaUuUaUaAg | | | UAAAUGCGAG | |
| | AgC*u*U | | | GcCu*U*u | | | CUG | |
| 1335 | G*g*CcUuA | 2911 | 2687-2707 | (VPmA)*C*aGc | 1337 | 2685-2707 | AAGGCCUUAU | 4048 |
| | uAaAuGcGa | | | UcGcAuUuAuAa | | | AAAUGCGAGC | |
| | GcU*g*U | | | GgCc*U*u | | | UG | |
| 1336 | G*c*CuUaU | 2912 | 2688-2708 | (VPmA)*A*cAg | 1338 | 2686-2708 | AGGCCUUAUA | 4049 |
| | aAaUgCgAg | | | CuCgCaUuUaUa | | | AAUGCGAGCU | |
| | CuG*u*U | | | AgGc*C*u | | | GUG | |
| 1337 | C*c*UuAuA | 2913 | 2689-2709 | (VPmG)*C*aCa | 1339 | 2687-2709 | GGCCUUAUAA | 4249 |
| | aAuGcGaGc | | | GcUcGcAuUuAu | | | AUGCGAGCUG | |
| | UgU*g*C | | | AaGg*C*c | | | UGC | |
| 1338 | C*u*UaUaA | 2914 | 2690-2710 | (VPmU)*G*cAc | 1340 | 2688-2710 | GCCUUAUAAA | 4250 |
| | aUgCgAgCu | | | AgCuCgCaUuUa | | | UGCGAGCUGU | |
| | GuG*c*A | | | UaAg*G*c | | | GCA | |
| 1339 | U*u*AuAaA | 2915 | 2691-2711 | (VPmU)*U*gCa | 1341 | 2689-2711 | GCCUUAUAAA | 4251 |
| | uGcGaGcUg | | | CaGcUcGcAuUu | | | UGCGAGCUGU | |
| | UgC*a*A | | | AuAa*G*g | | | GCAA | |
| 1340 | U*a*UaAaU | 2916 | 2692-2712 | (VPmG)*U*uGc | 1342 | 2690-2712 | CUUAUAAAUG | 4252 |
| | gCgAgCuGu | | | AcAgCuCgCaUu | | | CGAGCUGUGC | |
| | GcA*a*C | | | UaUa*A*g | | | AACU | |
| 1341 | A*u*AaAuG | 2917 | 2693-2713 | (VPmA)*G*uUg | 1343 | 2691-2713 | UUAUAAAUGC | 4253 |
| | cGaGcUgUg | | | CaCaGcUcGcAu | | | GAGCUGUGCA | |
| | CaA*c*U | | | UuAu*A*a | | | ACU | |
| 1342 | U*a*AaUgC | 2918 | 2694-2714 | (VPmU)*A*gUu | 1344 | 2692-2714 | UAUAAAUGCG | 4254 |
| | gAgCuGuGc | | | GcAcAgCuCgCa | | | AGCUGUGCAA | |
| | AaC*u*A | | | UuUa*U*a | | | CUA | |
| 1343 | A*a*AuGcG | 2919 | 2695-2715 | (VPmA)*U*aGu | 1345 | 2693-2715 | AUAAAUGCGA | 4255 |
| | aGcUgUgCa | | | UgCaCaGcUcGc | | | GCUGUGCAAC | |
| | AcU*a*U | | | AuUu*A*u | | | UAU | |
| 1344 | A*a*UgCgA | 2920 | 2696-2716 | (VPmA)*A*uAg | 1346 | 2694-2716 | UAAAUGCGAG | 4256 |
| | gCuGuGcAa | | | UuGcAcAgCuCg | | | CUGUGCAACU | |
| | CuA*u*U | | | CaUu*U*a | | | AUG | |
| 1345 | A*u*GcGaG | 2921 | 2697-2717 | (VPmG)*C*aUa | 1347 | 2695-2717 | AAAUGCGAGC | 4257 |
| | cUgUgCaAc | | | GuUgCaCaGcUc | | | UGUGCAACUA | |
| | UaU*g*C | | | GcAu*U*u | | | UGC | |
| 1346 | G*c*GaGcU | 2922 | 2699-2719 | (VPmA)*G*gCa | 1348 | 2697-2719 | AUGCGAGCUG | 4258 |
| | gUgCaAcUa | | | UaGuUgCaCaGc | | | UGCAACUAUG | |
| | UgC*c*U | | | UcGc*A*u | | | CCU | |
| 1347 | G*a*GcUgU | 2923 | 2701-2691 | (VPmA)*C*aGg | 1349 | 2669-2691 | GCGAGCUGUG | 4259 |
| | gCaAcUaUg | | | CaUaGuUgCaCa | | | CAACUAUGCC | |
| | CcU*g*U | | | GcUc*G*c | | | UGU | |
| 1348 | G*c*UgUgC | 2924 | 2703-2693 | (VPmG)*C*aCa | 1350 | 2671-2693 | GAGCUGUGCA | 4260 |
| | aAcUaUgCc | | | GgCaUaGuUgCa | | | ACUAUGCCUG | |
| | UgU*g*C | | | CaGc*U*c | | | UGC | |
| 1349 | G*g*GaAgG | 2925 | 2747-2767 | (VPmA)*C*aUu | 1351 | 2745-2767 | UGGGGAAGGA | 4261 |
| | aCgUuUaCa | | | UgUaAaCgUcCu | | | CGUUUACAAA | |
| | AaU*g*U | | | UcCc*C*a | | | UGU | |
| 1350 | G*g*AaGgA | 2926 | 2748-2768 | (VPmA)*A*cAu | 1352 | 2746-2768 | GGGGAAGGAC | 4262 |
| | cGuUuAcAa | | | UuGuAaAcGuCc | | | GUUUACAAAU | |
| | AuG*u*U | | | UuCc*C*c | | | GUG | |
| 1351 | U*u*GuAaG | 2927 | 2773-2793 | (VPmA)*C*gCu | 1353 | 2771-2793 | AUUUGUAAGA | 4263 |
| | aUgCcUuUu | | | AaAaGgCaUcUu | | | UGCCUUUUAG | |
| | AgC*g*U | | | AcAa*A*u | | | CGU | |
| 1352 | U*g*UaAgA | 2928 | 2774-2794 | (VPmA)*A*cGc | 1354 | 2772-2794 | UUUGUAAGAU | 4264 |
| | uGcCuUuUa | | | UaAaAgGcAuCu | | | GCCUUUUAGC | |
| | GcG*u*U | | | UaCa*A*a | | | GUG | |
| 1353 | G*u*AaGaU | 2929 | 2775-2795 | (VPmA)*C*aCg | 1355 | 2773-2795 | UUGUAAGAUG | 3562 |
| | gCcUuUuAg | | | CuAaAaGgCaUc | | | CCUUUUAGCG | |
| | CgU*g*U | | | UuAc*A*a | | | UGU | |
| 1354 | U*a*AgAuG | 2930 | 2776-2796 | (VPmU)*A*cAc | 1356 | 2774-2796 | UGUAAGAUGC | 4265 |
| | cCuUuUaGc | | | GcUaAaAgGcAu | | | CUUUUAGCGU | |
| | GuG*u*A | | | CuUa*C*a | | | GUA | |
| 1355 | A*a*GaUgC | 2931 | 2778-2798 | (VPmG)*U*aCa | 1357 | 2776-2798 | GUAAGAUGCC | 4266 |
| | cUuUuAgCg | | | CgCuAaAaGgCa | | | UUUUAGCGUG | |
| | UgU*a*C | | | UcUu*A*c | | | UAC | |
| 1356 | A*g*AuGcC | 2932 | 2779-2799 | (VPmU)*G*uAc | 1358 | 2777-2799 | UAAGAUGCCU | 4267 |
| | uUuUaGcGu | | | AcGcUaAaAgGc | | | UUUAGCGUGU | |
| | GuA*c*A | | | AuCu*U*a | | | ACA | |
| 1357 | A*u*GcCuU | 2933 | 2781-2801 | (VPmA)*C*uGu | 1359 | 2779-2801 | AGAUGCCUUU | 4268 |
| | uUaGcGuGu | | | AcAcGcUaAaAg | | | UAGCGUGUAC | |
| | AcA*g*U | | | GcAu*C*u | | | AGU | |
| 1358 | U*g*CcUuU | 2934 | 2782-2802 | (VPmU)*A*cUg | 1360 | 2780-2802 | GAUGCCUUUU | 4269 |
| | uAgCgUgUa | | | UaCaCgCuAaAa | | | AGCGUGUACA | |
| | CaG*u*A | | | GgCa*U*c | | | GUA | |
| 1359 | G*c*CuUuU | 2935 | 2783-2803 | (VPmG)*U*aCu | 1361 | 2781-2803 | AUGCCUUUUA | 4270 |
| | aGcGuGuAc | | | GuAcAcGcUaAa | | | GCGUGUACAG | |
| | AgU*a*C | | | AgGc*A*u | | | UAC | |
| 1360 | C*c*UuUuA | 2936 | 2784-2804 | (VPmG)*G*uAc | 1362 | 2782-2804 | UGCCUUUUAG | 4271 |
| | gCgUgUaCa | | | UgUaCaCgCuAa | | | CGUGUACAGU | |
| | GuA*c*C | | | AaGg*C*a | | | ACC | |
| 1361 | C*u*UuUaG | 2937 | 2785-2805 | (VPmG)*G*gUa | 1363 | 2783-2805 | GCCUUUUAGC | 4272 |
| | cGuGuAcAg | | | CuGuAcAcGcUa | | | GUGUACAGUA | |
| | UaC*c*C | | | AaAg*G*c | | | CCC | |
| 1362 | U*u*UuAgC | 2938 | 2786-2806 | (VPmA)*G*gGu | 1364 | 2784-2806 | CCUUUUAGCG | 4273 |
| | gUgUaCaGu | | | AcUgUaCaCgCu | | | UGUACAGUAC | |
| | AcC*c*U | | | AaAa*G*g | | | CCU | |
| 1363 | U*u*UaGcG | 2939 | 2787-2807 | (VPmA)*A*gGg | 1365 | 2785-2807 | CUUUUAGCGU | 4274 |
| | uGuAcAgUa | | | UaCuGuAcAcGc | | | GUACAGUACC | |
| | CcC*u*U | | | UaAa*A*g | | | CUG | |
| 1364 | U*u*AgCgU | 2940 | 2788-2808 | (VPmA)*C*aGg | 1366 | 2786-2808 | UUUUAGCGUG | 4275 |
| | gUaCaGuAc | | | GuAcUgUaCaCg | | | UACAGUACCC | |
| | CcU*g*U | | | CuAa*A*a | | | UGG | |
| 1365 | U*a*GcGuG | 2941 | 2789-2809 | (VPmU)*C*cAg | 1367 | 2787-2809 | UUUAGCGUGU | 4276 |
| | uAcAgUaCc | | | GgUaCuGuAcAc | | | ACAGUACCCU | |
| | CuG*g*A | | | GcUa*A*a | | | GGA | |
| 1366 | A*g*CgUgU | 2942 | 2790-2810 | (VPmA)*U*cCa | 1368 | 2788-2810 | UUAGCGUGUA | 4277 |
| | aCaGuAcCc | | | GgGuAcUgUaCa | | | CAGUACCCUG | |
| | UgG*a*U | | | CgCu*A*a | | | GAG | |
| 1367 | G*u*GuAcA | 2943 | 2792-2812 | (VPmU)*U*uCu | 1369 | 2790-2812 | UUAGCGUGUA | 4278 |
| | gUaCcCuGg | | | CcAgGgUaCuGu | | | CAGUACCCUG | |
| | AgA*a*A | | | AcAc*G*c | | | GAG | |
| 1368 | U*g*UaCaG | 2944 | 2793-2813 | (VPmG)*U*uUc | 1370 | 2791-2813 | CGUGUACAGU | 4279 |
| | uAcCcUgGa | | | UcCaGgGuAcUg | | | ACCCUGGAGA | |
| | GaA*a*C | | | UaCa*C*g | | | AAC | |
| 1369 | A*u*GgCaC | 2945 | 2824-2844 | (VPmA)*A*cAc | 1371 | 2822-2844 | AAAUGGCACA | 4280 |
| | aGuGaUcGa | | | UcGaUcAcUgUg | | | GUGAUCGAGU | |
| | GuG*u*U | | | CcAu*U*u | | | GUU | |
| 1370 | U*g*GcAcA | 2946 | 2825-2845 | (VPmA)*A*aCa | 1372 | 2823-2845 | AAUGGCACAG | 4281 |
| | gUgAuCgAg | | | CuCgAuCaCuGu | | | UGAUCGAGUG | |
| | UgU*u*U | | | GcCa*U*u | | | UUG | |
| 1371 | C*a*CaGuG | 2947 | 2828-2848 | (VPmA)*U*uCa | 1373 | 2826-2848 | GGCACAGUGA | 4282 |
| | aUcGaGuGu | | | AcAcUcGaUcAc | | | UCGAGUGUUG | |
| | UgA*a*U | | | UgUg*C*c | | | AAU | |
| 1372 | A*c*AgUgA | 2948 | 2829-2849 | (VPmU)*A*uUc | 1374 | 2827-2849 | GCACAGUGAU | 4283 |
| | uCgAgUgUu | | | AaCaCuCgAuCa | | | CGAGUGUUGA | |
| | GaA*u*A | | | CuGu*G*c | | | AUA | |
| 1373 | C*a*GuGaU | 2949 | 2830-2850 | (VPmU)*U*aUu | 1375 | 2828-2850 | CACAGUGAUC | 4284 |
| | cGaGuGuUg | | | CaAcAcUcGaUc | | | GAGUGUUGAA | |
| | AaU*a*A | | | AcUg*U*g | | | UAA | |
| 1374 | A*g*UgAuC | 2950 | 2831-2851 | (VPmA)*U*uAu | 1376 | 2829-2851 | ACAGUGAUCG | 4285 |
| | gAgUgUuGa | | | UcAaCaCuCgAu | | | AGUGUUGAAU | |
| | AuA*a*U | | | CaCu*G*u | | | AAU | |
| 1375 | G*u*GaUcG | 2951 | 2832-2852 | (VPmA)*A*uUa | 1377 | 2830-2852 | CAGUGAUCGA | 4286 |
| | aGuGuUgAa | | | UuCaAcAcUcGa | | | GUGUUGAAUA | |
| | UaA*u*U | | | UcAc*U*g | | | AUG | |
| 1376 | G*c*AgGgA | 2952 | 1351-1371 | (VPmU)*G*gGc | 1378 | 1349-1371 | UGGCAGGGAA | 4287 |
| | aCaCgUcUa | | | UaGaCgUgUuCc | | | CACGUCUAGC | |
| | GcC*c*A | | | CuGc*C*a | | | CCA | |
| 1377 | U*u*CaGaG | 2953 | 1547-1567 | (VPmG)*C*aCc | 1379 | 1545-1567 | AUUUCAGAGC | 4288 |
| | cAaCcUgGu | | | AcCaGgUuGcUc | | | AACCUGGUGG | |
| | GgU*g*C | | | UgAa*A*u | | | UGC | |
| 1378 | A*u*GaCgG | 2954 | 1684-1704 | (VPmG)*U*cGu | 1380 | 1682-1704 | CCAUGACGGU | 4289 |
| | uCaAgUcCg | | | CgGaCuUgAcCg | | | CAAGUCCGAC | |
| | AcG*a*C | | | UcAu*G*g | | | GAC | |
| 1379 | U*g*AcGgU | 2955 | 1685-1705 | (VPmA)*G*uCg | 1381 | 1683-1705 | CAUGACGGUC | 4290 |
| | cAaGuCcGa | | | UcGgAcUuGaCc | | | AAGUCCGACG | |
| | CgA*c*U | | | GuCa*U*g | | | ACG | |
| 1380 | A*c*GgUcA | 2956 | 1687-1707 | (VPmA)*C*cGu | 1382 | 1685-1707 | UGACGGUCAA | 4291 |
| | aGuCcGaCg | | | CgUcGgAcUuGa | | | GUCCGACGAC | |
| | AcG*g*U | | | CcGu*C*a | | | GGU | |
| 1381 | C*a*AgUcC | 2957 | 1692-1712 | (VPmG)*A*gAg | 1383 | 1690-1712 | GUCAAGUCCG | 4292 |
| | gAcGaCgGu | | | AcCgUcGuCgGa | | | ACGACGGUCU | |
| | CuC*u*C | | | CuUg*A*c | | | CUC | |
| 1382 | C*g*CuCaA | 2958 | 1769-1789 | (VPmU)*G*gCc | 1384 | 1767-1789 | CGCGCUCAAG | 4293 |
| | gUcCgUgGu | | | AcCaCgGaCuUg | | | UCCGUGGUGG | |
| | GgC*c*A | | | AgCg*C*g | | | CCA | |
| 1383 | U*c*AaGuC | 2959 | 1772-1792 | (VPmA)*C*uUg | 1385 | 1770-1792 | GCUCAAGUCC | 4294 |
| | cGuGgUgGc | | | GcCaCcAcGgAc | | | GUGGUGGCCA | |
| | CaA*g*U | | | UuGa*G*c | | | AGU | |
| 1384 | U*g*GuGgC | 2960 | 1781-1801 | (VPmA)*G*cUc | 1386 | 1779-1801 | CGUGGUGGCC | 4295 |
| | cAaGuUcAa | | | UuGaAcUuGgCc | | | AAGUUCAAGA | |
| | GaG*c*U | | | AcCa*C*g | | | GCG | |
| 1385 | G*g*UgGcC | 2961 | 1782-1802 | (VPmU)*C*gCu | 1387 | 1780-1802 | GUGGUGGCCA | 4296 |
| | aAgUuCaAg | | | CuUgAaCuUgGc | | | AGUUCAAGAG | |
| | AgC*g*A | | | CaCc*A*c | | | CGA | |
| 1386 | A*g*GaGgA | 2962 | 1847-1867 | (VPmA)*C*uCu | 1388 | 1845-1867 | AGAGGAGGAC | 4297 |
| | cGaCgAgGa | | | UcCuCgUcGuCc | | | GACGAGGAAG | |
| | AgA*g*U | | | UcCu*C*u | | | AGG | |
| 1387 | G*a*GgAcG | 2963 | 1849-1869 | (VPmU)*U*cCu | 1389 | 1847-1869 | AGGAGGACGA | 4298 |
| | aCgAgGaAg | | | CuUcCuCgUcGu | | | CGAGGAAGAG | |
| | AgG*a*A | | | CcUc*C*u | | | GAA | |
| 1388 | C*a*UcAaG | 2964 | 2289-2309 | (VPmA)*A*cUc | 1390 | 2287-2309 | CGCAUCAAGC | 4299 |
| | cUcGaGaAg | | | CuUcUcGaGcUu | | | UCGAGAAGGA | |
| | GaG*u*U | | | GaUg*C*g | | | GUU | |
| 1389 | A*u*CaAgC | 2965 | 2290-2310 | (VPmG)*A*aCu | 1391 | 2288-2310 | GCAUCAAGCU | 4300 |
| | uCgAgAaGg | | | CcUuCuCgAgCu | | | CGAGAAGGAG | |
| | AgU*u*C | | | UgAu*G*c | | | UUC | |
| 1390 | C*a*AgCuC | 2966 | 2292-2312 | (VPmU)*C*gAa | 1392 | 2290-2312 | AUCAAGCUCG | 4301 |
| | gAgAaGgAg | | | CuCcUuCuCgAg | | | AGAAGGAGUU | |
| | UuC*g*A | | | CuUg*A*u | | | CGA | |
| 1391 | U*c*CuUaG | 2967 | 2402-2422 | (VPmU)*G*gAg | 1393 | 2400-2422 | CUUCCUUAGC | 4302 |
| | cUuCgGaGa | | | UcUcCgAaGcUa | | | UUCGGAGACU | |
| | CuC*c*A | | | AgGa*A*g | | | CCA | |
| 1392 | C*c*UuAgC | 2968 | 2403-2423 | (VPmA)*U*gGa | 1394 | 2401-2423 | UUCCUUAGCU | 4303 |
| | uUcGgAgAc | | | GuCuCcGaAgCu | | | UCGGAGACUC | |
| | UcC*a*U | | | AaGg*A*a | | | CAG | |
| 1393 | U*a*GcUuC | 2969 | 2406-2426 | (VPmU)*G*uCu | 1395 | 2404-2426 | CUUAGCUUCG | 4304 |
| | gGaGaCuCc | | | GgAgUcUcCgAa | | | GAGACUCCAG | |
| | AgA*c*A | | | GcUa*A*g | | | ACA | |
| 1394 | A*g*CuUcG | 2970 | 2407-2427 | (VPmU)*U*gUc | 1396 | 2405-2427 | UUAGCUUCGG | 4305 |
| | gAgAcUcCa | | | UgGaGuCuCcGa | | | AGACUCCAGA | |
| | GaC*a*A | | | AgCu*A*a | | | CAA | |
| 1395 | G*c*UuCgG | 2971 | 2408-2428 | (VPmA)*U*uGu | 1397 | 2406-2428 | UAGCUUCGGA | 4306 |
| | aGaCuCcAg | | | CuGgAgUcUcCg | | | GACUCCAGAC | |
| | AcA*a*U | | | AaGc*U*a | | | AAU | |
| 1396 | C*u*UcGgA | 2972 | 2409-2429 | (VPmG)*A*uUg | 1398 | 2407-2429 | AGCUUCGGAG | 4307 |
| | gAcUcCaGa | | | UcUgGaGuCuCc | | | ACUCCAGACA | |
| | CaA*u*C | | | GaAg*C*u | | | AUC | |
| 1397 | U*u*GcCuC | 2973 | 2410-2430 | (VPmA)*G*uGc | 1399 | 2408-2430 | UUUUGCCUCC | 4308 |
| | cUcGuCgGa | | | UcCgAcGaGgAg | | | UCGUCGGAGC | |
| | GcA*c*U | | | GcAa*A*a | | | ACU | |
| 1398 | U*g*CcUcC | 2974 | 2411-2431 | (VPmG)*A*gUg | 1400 | 2409-2431 | UUUGCCUCCU | 4309 |
| | uCgUcGgAg | | | CuCcGaCgAgGa | | | CGUCGGAGCA | |
| | CaC*u*C | | | GgCa*A*a | | | CUC | |

| | | | | | | |
|---|---|---|---|---|---|---|
| 1399 | a*a*ccagA | 2975 | — | (VPmU)*C*aac | 1401 | See Table 2. |
| | cCACggccc | | | GggccgugGuCu | | |
| | guu*g*a | | | gguu*c*a | | |
| 1400 | a*c*cagaC | 2976 | — | (VPmU)*C*caa | 1402 | See Table 2. |
| | cACGgcccg | | | CgggccguGgUc | | |
| | uug*g*a | | | uggu*u*c | | |
| 1401 | g*g*acauU | 2977 | — | (VPmU)*C*gau | 1403 | See Table 2. |
| | cUUAuuuuu | | | AaaaauaaGaAu | | |
| | auc*g*a | | | gucc*c*c | | |
| 1402 | g*a*cauuC | 2978 | — | (VPmU)*U*cga | 1404 | See Table 2. |
| | uUAUuuuua | | | UaaaaauaAgAa | | |
| | ucg*a*a | | | uguc*c*c | | |
| 1403 | c*a*uucuU | 2979 | — | (VPmU)*G*cuc | 1405 | See Table 2. |
| | aUUUuuauc | | | GauaaaaaUaAg | | |
| | gag*c*a | | | aaug*u*c | | |
| 1404 | u*u*cuuaU | 2980 | — | (VPmU)*G*ugc | 1406 | See Table 2. |
| | uUUUaucga | | | UcgauaaaAaUa | | |
| | gca*c*a | | | agaa*u*g | | |
| 1405 | u*c*uuauU | 2981 | — | (VPmU)*U*gug | 1407 | See Table 2. |
| | uUUAucgag | | | CucgauaaAaAu | | |
| | cac*a*a | | | aaga*a*u | | |
| 1406 | c*u*uauuU | 2982 | — | (VPmU)*U*ugu | 1408 | See Table 2. |
| | uUAUcgagc | | | GcucgauaAaAa | | |
| | aca*a*a | | | uaag*a*a | | |
| 1407 | u*u*auuuU | 2983 | — | (VPmU)*U*uug | 1409 | See Table 2. |
| | uAUCgagca | | | UgcucgauAaAa | | |
| | caa*a*a | | | auaa*g*a | | |
| 1408 | u*a*uuuuU | 2984 | — | (VPmU)*G*uuu | 1410 | See Table 2. |
| | aUCGagcac | | | GugcucgaUaAa | | |
| | aaa*c*a | | | aaua*a*g | | |
| 1409 | a*u*uuuuA | 2985 | — | (VPmU)*C*guu | 1411 | See Table 2. |
| | uCGAgcaca | | | UgugcucgAuAa | | |
| | aac*g*a | | | aaau*a*a | | |
| 1410 | u*u*uuuaU | 2986 | — | (VPmU)*C*cgu | 1412 | See Table 2. |
| | cGAGcacaa | | | UugugcucGaUa | | |
| | acg*g*a | | | aaaa*u*a | | |
| 1411 | u*u*uuauC | 2987 | — | (VPmU)*U*ccg | 1413 | See Table 2. |
| | gAGCacaaa | | | UuugugcuCgAu | | |
| | cgg*a*a | | | aaaa*a*u | | |
| 1412 | u*u*uaucG | 2988 | — | (VPmU)*U*ucc | 1414 | See Table 2. |
| | aGCAcaaac | | | GuuugugcUcGa | | |
| | gga*a*a | | | uaaa*a*a | | |
| 1413 | u*u*aucgA | 2989 | — | (VPmU)*U*uuc | 1415 | See Table 2. |
| | gCACaaacg | | | CguuugugCuCg | | |
| | gaa*a*a | | | auaa*a*a | | |
| 1414 | c*c*uuccC | 2990 | — | (VPmU)*U*cga | 1416 | See Table 2. |
| | cUUCaccaa | | | UuggugaaGgGg | | |
| | ucg*a*a | | | aagg*u*g | | |
| 1415 | c*u*ucacC | 2991 | — | (VPmU)*U*uuc | 1417 | See Table 2. |
| | aAUCgagau | | | AucucgauUgGu | | |
| | gaa*a*a | | | gaag*g*g | | |
| 1416 | a*a*agcaU | 2992 | — | (VPmU)*U*cca | 1418 | See Table 2. |
| | cCAAucccg | | | CgggauugGaUg | | |
| | ugg*a*a | | | cuuu*u*u | | |
| 1417 | g*c*auccA | 2993 | — | (VPmU)*A*ccu | 1419 | See Table 2. |
| | aUCCcgugg | | | CcacgggaUuGg | | |
| | agg*u*a | | | augc*u*u | | |
| 1418 | u*c*acgcC | 2994 | — | (VPmU)*A*ucg | 1420 | See Table 2. |
| | aGAGgauga | | | UcauccucUgGc | | |
| | cga*u*a | | | guga*c*c | | |
| 1419 | a*u*gacgA | 2995 | — | (VPmU)*C*guu | 1421 | See Table 2. |
| | uUGUuuauc | | | GauaaacaAuCg | | |
| | aac*g*a | | | ucau*c*c | | |
| 1420 | u*g*acgaU | 2996 | — | (VPmU)*A*cgu | 1422 | See Table 2. |
| | uGUUuauca | | | UgauaaacAaUc | | |
| | acg*u*a | | | guca*u*c | | |
| 1421 | u*u*guuuA | 2997 | — | (VPmU)*U*aga | 1423 | See Table 2. |
| | uCAAcguca | | | UgacguugAuAa | | |
| | ucu*a*a | | | acaa*u*c | | |
| 1422 | u*a*ugccC | 2998 | — | (VPmU)*C*aaa | 1424 | See Table 2. |
| | cGCAgggua | | | UacccugcGgGg | | |
| | uuu*g*a | | | caua*u*u | | |
| 1423 | c*g*caggG | 2999 | — | (VPmU)*A*ucu | 1425 | See Table 2. |
| | uAUUuguaa | | | UuacaaauAcCc | | |
| | aga*u*a | | | ugcg*g*g | | |
| 1424 | g*c*agggU | 3000 | — | (VPmU)*C*auc | 1426 | See Table 2. |
| | aUUUguaaa | | | UuuacaaaUaCc | | |
| | gau*g*a | | | cugc*g*g | | |
| 1425 | c*a*ggguA | 3001 | — | (VPmU)*U*cau | 1427 | See Table 2. |
| | uUUGuaaag | | | CuuuacaaAuAc | | |
| | aug*a*a | | | ccug*c*g | | |
| 1426 | a*g*gguaU | 3002 | — | (VPmU)*C*uca | 1428 | See Table 2. |
| | uUGUaaaga | | | UcuuuacaAaUa | | |
| | uga*g*a | | | cccu*g*c | | |
| 1427 | g*g*uauuU | 3003 | — | (VPmU)*G*gcu | 1429 | See Table 2. |
| | gUAAagaug | | | CaucuuuaCaAa | | |
| | agc*c*a | | | uacc*u*g | | |
| 1428 | g*u*auuuG | 3004 | — | (VPmU)*G*ggc | 1430 | See Table 2. |
| | uAAAgauga | | | UcaucuuuAcAa | | |
| | gcc*c*a | | | auaccu | | |
| 1429 | u*a*uuugU | 3005 | — | (VPmU)*U*ggg | 1431 | See Table 2. |
| | aAAGaugag | | | CucaucuuUaCa | | |
| | ccc*a*a | | | aaua*c*c | | |
| 1430 | a*u*uuguA | 3006 | — | (VPmU)*C*ugg | 1432 | See Table 2. |
| | aAGAugagc | | | GcucaucuUuAc | | |
| | cca*g*a | | | aaau*a*c | | |
| 1431 | u*u*uguaA | 3007 | — | (VPmU)*G*cug | 1433 | See Table 2. |
| | aGAUgagcc | | | GgcucaucUuUa | | |
| | cag*c*a | | | caaa*u*a | | |
| 1432 | u*u*guaaA | 3008 | — | (VPmU)*U*gcu | 1434 | See Table 2. |
| | gAUGagccc | | | GggcucauCuUu | | |
| | agc*a*a | | | acaa*a*u | | |
| 1433 | u*g*uaaaG | 3009 | — | (VPmU)*C*ugc | 1435 | See Table 2. |
| | aUGAgccca | | | UgggcucaUcUu | | |
| | gca*g*a | | | uaca*a*a | | |
| 1434 | g*u*aaagA | 3010 | — | (VPmU)*G*cug | 1436 | See Table 2. |
| | uGAGcccag | | | CugggcucAuCu | | |
| | cag*c*a | | | uuac*a*a | | |
| 1435 | u*a*aagaU | 3011 | — | (VPmU)*A*gcu | 1437 | See Table 2. |
| | gAGCccagc | | | GcugggcuCaUc | | |
| | agc*u*a | | | uuua*c*a | | |
| 1436 | a*a*gaugA | 3012 | — | (VPmU)*G*uag | 1438 | See Table 2. |
| | gCCCagcag | | | CugcugggCuCa | | |
| | cua*c*a | | | ucuu*u*a | | |
| 1437 | a*g*augaG | 3013 | — | (VPmU)*U*gua | 1439 | See Table 2. |
| | cCCAgcagc | | | GcugcuggGcUc | | |
| | uac*a*a | | | aucu*u*u | | |
| 1438 | g*a*ugagC | 3014 | — | (VPmU)*G*ugu | 1440 | See Table 2. |
| | cCAGcagcu | | | AgcugcugGgCu | | |
| | aca*c*a | | | cauc*u*u | | |
| 1439 | a*u*gagcC | 3015 | — | (VPmU)*U*gug | 1441 | See Table 2. |
| | cAGCagcua | | | UagcugcuGgGc | | |
| | cac*a*a | | | ucau*c*u | | |
| 1440 | u*g*agccC | 3016 | — | (VPmU)*A*ugu | 1442 | See Table 2. |
| | aGCAgcuac | | | GuagcugcUgGg | | |
| | aca*u*a | | | cuca*u*c | | |
| 1441 | a*c*acauG | 3017 | — | (VPmU)*U*uug | 1443 | See Table 2. |
| | uACAacuug | | | CaaguuguAcAu | | |
| | caa*a*a | | | gugu*a*g | | |
| 1442 | a*c*auguA | 3018 | — | (VPmU)*U*guu | 1444 | See Table 2. |
| | cAACuugca | | | UgcaaguuGuAc | | |
| | aac*a*a | | | augu*g*u | | |
| 1443 | u*g*caacA | 3019 | — | (VPmU)*A*gug | 1445 | See Table 2. |
| | cGCAcagaa | | | UucugugcGuGu | | |
| | cac*u*a | | | ugca*a*g | | |
| 1444 | g*c*aacaC | 3020 | — | (VPmU)*G*agu | 1446 | See Table 2. |
| | gCACagaac | | | GuucugugCgUg | | |
| | acu*c*a | | | uugc*a*a | | |
| 1445 | c*a*acacG | 3021 | — | (VPmU)*U*gag | 1447 | See Table 2. |
| | cACAgaaca | | | UguucuguGcGu | | |
| | cuc*a*a | | | guug*c*a | | |
| 1446 | a*a*cacgC | 3022 | — | (VPmU)*A*uga | 1448 | See Table 2. |
| | aCAGaacac | | | GuguucugUgCg | | |
| | uca*u*a | | | uguu*g*c | | |
| 1447 | a*c*acgcA | 3023 | — | (VPmU)*C*aug | 1449 | See Table 2. |
| | cAGAacacu | | | AguguucuGuGc | | |
| | cau*g*a | | | gugu*u*g | | |
| 1448 | c*a*cgcaC | 3024 | — | (VPmU)*C*cau | 1450 | See Table 2. |
| | aGAAcacuc | | | GaguguucUgUg | | |
| | aug*g*a | | | cgug*u*u | | |
| 1449 | c*c*cugaC | 3025 | — | (VPmU)*A*cca | 1451 | See Table 2. |
| | cCCGcgggu | | | AcccgcggGgUc | | |
| | ugg*u*a | | | aggg*g*a | | |
| 1450 | c*u*gaccC | 3026 | — | (VPmU)*A*uac | 1452 | See Table 2. |
| | cGCGgguug | | | CaacccgcGgGg | | |
| | gua*u*a | | | ucag*g*g | | |
| 1451 | g*a*ccccG | 3027 | — | (VPmU)*G*gau | 1453 | See Table 2. |
| | cGGGuuggu | | | AccaacccGcGg | | |
| | auc*c*a | | | gguc*a*g | | |
| 1452 | a*c*cccgC | 3028 | — | (VPmU)*G*gga | 1454 | See Table 2. |
| | gGGUuggua | | | UaccaaccCgCg | | |
| | ucc*c*a | | | gggu*c*a | | |
| 1453 | g*u*auccC | 3029 | — | (VPmU)*A*ccu | 1455 | See Table 2. |
| | uUCAggacu | | | AguccugaAgGg | | |
| | agg*u*a | | | auac*c*a | | |
| 1454 | c*a*gacaA | 3030 | — | (VPmU)*G*uua | 1456 | See Table 2. |
| | uAACcccuu | | | AagggguuAuUg | | |
| | uaa*c*a | | | ucug*c*a | | |
| 1455 | a*g*acaaU | 3031 | — | (VPmU)*G*guu | 1457 | See Table 2. |
| | aACCccuuu | | | AaagggguUaUu | | |
| | aac*c*a | | | gucu*g*c | | |
| 1456 | a*a*uaccA | 3032 | — | (VPmU)*U*cga | 1458 | See Table 2. |
| | gGAUcagua | | | UacugaucCuGg | | |
| | ucg*a*a | | | uauu*c*u | | |
| 1457 | c*c*uguuU | 3033 | — | (VPmU)*U*cgg | 1459 | See Table 2. |
| | aGUCcacca | | | UgguggacUaAa | | |
| | ccg*a*a | | | cagg*g*g | | |
| 1458 | c*a*agucC | 3034 | — | (VPmU)*A*cuc | 1460 | See Table 2. |
| | aAGUcaugc | | | GcaugacuUgGa | | |
| | gag*u*a | | | cuug*a*c | | |
| 1459 | a*a*guccA | 3035 | — | (VPmU)*A*acu | 1461 | See Table 2. |
| | aGUCaugcg | | | CgcaugacUuGg | | |
| | agu*u*a | | | acuu*g*a | | |
| 1460 | g*u*gcgaC | 3036 | — | (VPmU)*G*ggu | 1462 | See Table 2. |
| | cACGcgugc | | | GcacgcguGgUc | | |
| | acc*c*a | | | gcac*a*g | | |
| 1461 | c*c*augaC | 3037 | — | (VPmU)*G*ucg | 1463 | See Table 2. |
| | gGUCaaguc | | | GacuugacCgUc | | |
| | cga*c*a | | | augg*g*g | | |
| 1462 | a*u*gacgG | 3038 | — | (VPmU)*U*cgu | 1464 | See Table 2. |
| | uCAAguccg | | | CggacuugAcCg | | |
| | acg*a*a | | | ucau*g*g | | |
| 1463 | u*g*acggU | 3039 | — | (VPmU)*G*ucg | 1465 | See Table 2. |
| | cAAGuccga | | | UcggacuuGaCc | | |
| | cga*c*a | | | guca*u*g | | |
| 1464 | g*a*cgguC | 3040 | — | (VPmU)*C*guc | 1466 | See Table 2. |
| | aAGUccgac | | | GucggacuUgAc | | |
| | gac*g*a | | | cguc*a*u | | |
| 1465 | a*c*ggucA | 3041 | — | (VPmU)*C*cgu | 1467 | See Table 2. |
| | aGUCcgacg | | | CgucggacUuGa | | |
| | acg*g*a | | | ccgu*c*a | | |
| 1466 | c*g*gucaA | 3042 | — | (VPmU)*A*ccg | 1468 | See Table 2. |
| | gUCCgacga | | | UcgucggaCuUg | | |
| | cgg*u*a | | | accg*u*c | | |
| 1467 | g*g*ucaaG | 3043 | — | (VPmU)*G*acc | 1469 | See Table 2. |
| | uCCGacgac | | | GucgucggAcUu | | |
| | ggu*c*a | | | gacc*g*u | | |
| 1468 | c*a*agucC | 3044 | — | (VPmU)*A*gag | 1470 | See Table 2. |
| | gACGacggu | | | AccgucguCgGa | | |
| | cuc*u*a | | | cuug*a*c | | |
| 1469 | a*g*cagcG | 3045 | — | (VPmU)*A*cgg | 1471 | See Table 2. |
| | cGCUcaagu | | | AcuugagcGcGc | | |
| | ccg*u*a | | | ugcu*g*g | | |
| 1470 | g*c*agcgC | 3046 | — | (VPmU)*C*acg | 1472 | See Table 2. |
| | gCUCaaguc | | | GacuugagCgCg | | |
| | cgu*g*a | | | cugc*u*g | | |
| 1471 | g*a*gaacG | 3047 | — | (VPmU)*A*uca | 1473 | See Table 2. |
| | aCCCcaacc | | | GguuggggUcGu | | |
| | uga*u*a | | | ucuc*g*c | | |
| 1472 | c*u*gacgG | 3048 | — | (VPmU)*A*ccc | 1474 | See Table 2. |
| | aGAGcgaga | | | UcucgcucUcCg | | |
| | ggg*u*a | | | ucag*c*u | | |
| 1473 | a*g*agcgA | 3049 | — | (VPmU)*G*uag | 1475 | See Table 2. |
| | gAGGgugga | | | UccacccuCuCg | | |
| | cua*c*a | | | cucu*c*c | | |
| 1474 | g*c*gagaG | 3050 | — | (VPmU)*G*ccg | 1476 | See Table 2. |
| | gGUGgacua | | | UaguccacCcUc | | |
| | cgg*c*a | | | ucgc*u*c | | |
| 1475 | c*g*agagG | 3051 | — | (VPmU)*A*gcc | 1477 | See Table 2. |
| | gUGGacuac | | | GuaguccaCcCu | | |
| | ggc*u*a | | | cucg*c*u | | |
| 1476 | g*a*ggguG | 3052 | — | (VPmU)*C*gaa | 1478 | See Table 2. |
| | gACUacggc | | | GccguaguCcAc | | |
| | uuc*g*a | | | ccuc*u*c | | |
| 1477 | c*a*cgagA | 3053 | — | (VPmU)*C*ccc | 1479 | See Table 2. |
| | aCAGcucgc | | | GcgagcugUuCu | | |
| | ggg*g*a | | | cgug*g*u | | |
| 1478 | g*g*gcgaG | 3054 | — | (VPmU)*C*gcg | 1480 | See Table 2. |
| | aAGCauaag | | | CuuaugcuUcUc | | |
| | cgc*g*a | | | gccc*a*g | | |
| 1479 | g*a*gaagC | 3055 | — | (VPmU)*U*ggc | 1481 | See Table 2. |
| | aUAAgcgcg | | | CgcgcuuaUgCu | | |
| | gcc*a*a | | | ucuc*g*c | | |
| 1480 | c*a*caggG | 3056 | — | (VPmU)*U*cgu | 1482 | See Table 2. |
| | aCACuugcg | | | CgcaagugUcCc | | |
| | acg*a*a | | | ugug*g*c | | |
| 1481 | u*g*cgacG | 3057 | — | (VPmU)*G*cca | 1483 | See Table 2. |
| | aAGAcucgg | | | CcgagucuUcGu | | |
| | ugg*c*a | | | cgca*a*g | | |
| 1482 | g*a*cucgG | 3058 | — | (VPmU)*G*acu | 1484 | See Table 2. |
| | uGGCcggcg | | | CgccggccAcCg | | |
| | agu*c*a | | | aguc*u*u | | |
| 1483 | g*c*cggcG | 3059 | — | (VPmU)*A*ugc | 1485 | See Table 2. |
| | aGUCggacc | | | GguccgacUcGc | | |
| | gca*u*a | | | cggc*c*a | | |
| 1484 | c*g*gcgaG | 3060 | — | (VPmU)*C*uau | 1486 | See Table 2. |
| | uCGGaccgc | | | GcgguccgAcUc | | |
| | aua*g*a | | | gccg*g*c | | |
| 1485 | g*g*cgagU | 3061 | — | (VPmU)*U*cua | 1487 | See Table 2. |
| | cGGAccgca | | | UgcgguccGaCu | | |
| | uag*a*a | | | cgcc*g*g | | |
| 1486 | g*a*gucgG | 3062 | — | (VPmU)*U*cgu | 1488 | See Table 2. |
| | aCCGcauag | | | CuaugcggUcCg | | |
| | acg*a*a | | | acuc*g*c | | |
| 1487 | a*g*ucggA | 3063 | — | (VPmU)*A*ucg | 1489 | See Table 2. |
| | cCGCauaga | | | UcuaugcgGuCc | | |
| | cga*u*a | | | gacu*c*g | | |
| 1488 | g*u*cggaC | 3064 | — | (VPmU)*C*auc | 1490 | See Table 2. |
| | cGCAuagac | | | GucuaugcGgUc | | |
| | gau*g*a | | | cgac*u*c | | |
| 1489 | g*a*gacuC | 3065 | — | (VPmU)*A*ggc | 1491 | See Table 2. |
| | cAGAcaauc | | | GauugucuGgAg | | |
| | gcc*u*a | | | ucuc*c*g | | |
| 1490 | g*c*cuuuU | 3066 | — | (VPmU)*C*cga | 1492 | See Table 2. |
| | gCCUccucg | | | CgaggaggCaAa | | |
| | ucg*g*a | | | aggc*g*a | | |
| 1491 | c*u*cgucG | 3067 | — | (VPmU)*C*cga | 1493 | See Table 2. |
| | gAGCacucc | | | GgagugcuCcGa | | |
| | ucg*g*a | | | cgag*g*a | | |
| 1492 | c*g*ucggA | 3068 | — | (VPmU)*C*ucc | 1494 | See Table 2. |
| | gCACuccuc | | | GaggagugCuCc | | |
| | gga*g*a | | | gacg*a*g | | |
| 1493 | g*a*gcacU | 3069 | — | (VPmU)*C*cgu | 1495 | See Table 2. |
| | cCUCggaga | | | UcuccgagGaGu | | |
| | acg*g*a | | | gcuc*c*g | | |
| 1494 | g*a*gcacG | 3070 | — | (VPmU)*C*acu | 1496 | See Table 2. |
| | cCCCauauu | | | AauaugggGcGu | | |
| | agu*g*a | | | gcuc*c*c | | |
| 1495 | a*g*cacgC | 3071 | — | (VPmU)*C*cac | 1497 | See Table 2. |
| | cCCAuauua | | | UaauauggGgCg | | |
| | gug*g*a | | | ugcu*c*c | | |
| 1496 | c*a*cgccC | 3072 | — | (VPmU)*G*acc | 1498 | See Table 2. |
| | cAUAuuagu | | | AcuaauauGgGg | | |
| | ggu*c*a | | | cgug*c*u | | |
| 1497 | c*c*cauaU | 3073 | — | (VPmU)*G*ccc | 1499 | See Table 2. |
| | uAGUggucc | | | GgaccacuAaUa | | |
| | ggg*c*a | | | uggg*g*c | | |
| 1498 | u*a*uuagU | 3074 | — | (VPmU)*C*cgg | 1500 | See Table 2. |
| | gGUCcgggc | | | GcccggacCaCu | | |
| | ccg*g*a | | | aaua*u*g | | |
| 1499 | u*u*uguaA | 3075 | — | (VPmU)*G*cua | 1501 | See Table 2. |
| | gAUGccuuu | | | AaaggcauCuUa | | |
| | uag*c*a | | | caaa*u*u | | |
| 1500 | u*u*guaaG | 3076 | — | (VPmU)*C*gcu | 1502 | See Table 2. |
| | aUGCcuuuu | | | AaaaggcaUcUu | | |
| | agc*g*a | | | acaa*a*u | | |
| 1501 | u*g*uaagA | 3077 | — | (VPmU)*A*cgc | 1503 | See Table 2. |
| | uGCCuuuua | | | UaaaaggcAuCu | | |
| | gcg*u*a | | | uaca*a*a | | |
| 1502 | a*a*gaugC | 3078 | — | (VPmU)*U*aca | 1504 | See Table 2. |
| | cUUUuagcg | | | CgcuaaaaGgCa | | |
| | ugu*a*a | | | ucuu*a*c | | |
| 1503 | c*u*uuuaG | 3079 | — | (VPmU)*G*gua | 1505 | See Table 2. |
| | cGUGuacag | | | CuguacacGcUa | | |
| | uac*c*a | | | aaag*g*c | | |
| 1504 | u*u*uuagC | 3080 | — | (VPmU)*G*ggu | 1506 | See Table 2. |
| | gUGUacagu | | | AcuguacaCgCu | | |
| | acc*c*a | | | aaaa*g*g | | |
| 1505 | g*u*acagU | 3081 | — | (VPmU)*G*uuu | 1507 | See Table 2. |
| | aCCCuggag | | | CuccagggUaCu | | |
| | aaa*c*a | | | guac*a*c | | |
| 1506 | u*a*caguA | 3082 | — | (VPmU)*U*guu | 1508 | See Table 2. |
| | cCCUggaga | | | UcuccaggGuAc | | |
| | aac*a*a | | | ugua*c*a | | |
| 1507 | a*c*aguaC | 3083 | — | (VPmU)*G*ugu | 1509 | See Table 2. |
| | cCUGgagaa | | | UucuccagGgUa | | |
| | aca*c*a | | | cugu*a*c | | |
| 1508 | c*a*guacC | 3084 | — | (VPmU)*U*gug | 1510 | See Table 2. |
| | cUGGagaaa | | | UuucuccaGgGu | | |
| | cac*a*a | | | acug*u*a | | |
| 1509 | a*g*uaccC | 3085 | — | (VPmU)*A*ugu | 1511 | See Table 2. |
| | uGGAgaaac | | | GuuucuccAgGg | | |
| | aca*u*a | | | uacu*g*u | | |
| 1510 | g*u*acccU | 3086 | — | (VPmU)*C*aug | 1512 | See Table 2. |
| | gGAGaaaca | | | UguuucucCaGg | | |
| | cau*g*a | | | guac*u*g | | |
| 1511 | u*a*cccuG | 3087 | — | (VPmU)*U*cau | 1513 | See Table 2. |
| | gAGAaacac | | | GuguuucuCcAg | | |
| | aug*a*a | | | ggua*c*u | | |
| 1512 | a*c*ccugG | 3088 | — | (VPmU)*U*uca | 1514 | See Table 2. |
| | aGAAacaca | | | UguguuucUcCa | | |
| | uga*a*a | | | gggu*a*c | | |
| 1513 | c*c*cuggA | 3089 | — | (VPmU)*U*uuc | 1515 | See Table 2. |
| | gAAAcacau | | | AuguguuuCuCc | | |
| | gaa*a*a | | | aggg*u*a | | |
| 1514 | c*c*uggaG | 3090 | — | (VPmU)*U*uuu | 1516 | See Table 2. |
| | aAACacaug | | | CauguguuUcUc | | |
| | aaa*a*a | | | cagg*g*u | | |
| 1515 | c*u*ggagA | 3091 | — | (VPmU)*U*uuu | 1517 | See Table 2. |
| | aACAcauga | | | UcauguguUuCu | | |
| | aaa*a*a | | | ccag*g*g | | |
| 1516 | u*g*gagaA | 3092 | — | (VPmU)*U*uuu | 1518 | See Table 2. |
| | aCACaugaa | | | UucaugugUuUc | | |
| | aaa*a*a | | | ucca*g*g | | |
| 1517 | g*g*agaaA | 3093 | — | (VPmU)*A*uuu | 1519 | See Table 2. |
| | cACAugaaa | | | UuucauguGuUu | | |
| | aaa*u*a | | | cucc*a*g | | |
| 1518 | a*u*ggcaC | 3094 | — | (VPmU)*A*cac | 1520 | See Table 2. |
| | aGUGaucga | | | UcgaucacUgUg | | |
| | gug*u*a | | | ccau*u*u | | |
| 1519 | a*a*caguC | 3095 | — | (VPmU)*G*ggu | 1521 | See Table 2. |
| | uUUGgauua | | | UaauccaaAgAc | | |
| | acc*c*a | | | uguu*u*u | | |
| 1520 | g*g*auuaA | 3096 | — | (VPmU)*U*guc | 1522 | See Table 2. |
| | cCCUcuaua | | | UauagaggGuUa | | |
| | gac*a*a | | | aucc*a*a | | |
| 1521 | a*u*agacA | 3097 | — | (VPmU)*G*ugc | 1523 | See Table 2. |
| | gAAUagaua | | | UaucuauuCuGu | | |
| | gca*c*a | | | cuau*a*g | | |
| 1522 | u*a*auaaA | 3098 | — | (VPmU)*C*uag | 1524 | See Table 2. |
| | cCUUaggaa | | | UuccuaagGuUu | | |
| | cua*g*a | | | auua*c*c | | |
| 1523 | a*u*gaccC | 3099 | — | (VPmU)*A*cua | 1525 | See Table 2. |
| | aUUAugucc | | | GgacauaaUgGg | | |
| | uag*u*a | | | ucau*c*u | | |
| 1524 | u*a*uaccA | 3100 | — | (VPmU)*G*uag | 1526 | See Table 2. |
| | gUAUaaaag | | | CuuuuauaCuGg | | |
| | cua*c*a | | | uaua*a*u | | |
| 1525 | a*a*agccC | 3101 | — | (VPmU)*U*aau | 1527 | See Table 2. |
| | uGGAacgca | | | UgcguuccAgGg | | |
| | auu*a*a | | | cuuu*u*g | | |
| 1526 | a*a*uuaaA | 3102 | — | (VPmU)*C*cuu | 1528 | See Table 2. |
| | uACAcuagu | | | AcuaguguAuUu | | |
| | aag*g*a | | | aauu*g*c | | |
| 1527 | a*u*uaaaU | 3103 | — | (VPmU)*U*ccu | 1529 | See Table 2. |
| | aCACuagua | | | UacuagugUaUu | | |
| | agg*a*a | | | uaau*u*g | | |
| 1528 | u*a*ugauU | 3104 | — | (VPmU)*C*guu | 1530 | See Table 2. |
| | uAUUagcac | | | GugcuaauAaAu | | |
| | aac*g*a | | | caua*u*u | | |
| 1529 | a*u*gauuU | 3105 | — | (VPmU)*A*cgu | 1531 | See Table 2. |
| | aUUAgcaca | | | UgugcuaaUaAa | | |
| | acg*u*a | | | ucau*a*u | | |
| 1530 | g*a*uuuaU | 3106 | — | (VPmU)*C*cac | 1532 | See Table 2. |
| | uAGCacaac | | | GuugugcuAaUa | | |
| | gug*g*a | | | aauc*a*u | | |
| 1531 | a*u*uuauU | 3107 | — | (VPmU)*A*cca | 1533 | See Table 2. |
| | aGCAcaacg | | | CguugugcUaAu | | |
| | ugg*u*a | | | aaau*c*a | | |
| 1532 | u*u*auuaG | 3108 | — | (VPmU)*G*uac | 1534 | See Table 2. |
| | cACAacgug | | | CacguuguGcUa | | |
| | gua*c*a | | | auaa*a*u | | |
| 1533 | u*a*gcacA | 3109 | — | (VPmU)*A*aua | 1535 | See Table 2. |
| | aCGUgguac | | | GuaccacgUuGu | | |
| | uau*u*a | | | gcua*a*u | | |
| 1534 | u*u*uuuuA | 3110 | — | (VPmU)*G*aug | 1536 | See Table 2. |
| | uUAAgcuag | | | CuagcuuaAuAa | | |
| | cau*c*a | | | aaaa*g*a | | |
| 1535 | u*u*uauuA | 3111 | — | (VPmU)*G*cag | 1537 | See Table 2. |
| | aGCUagcau | | | AugcuagcUuAa | | |
| | cug*c*a | | | uaaa*a*a | | |
| 1536 | u*u*uucuC | 3112 | — | (VPmU)*G*uau | 1538 | See Table 2. |
| | uAAUcagag | | | CucugauuAgAg | | |
| | aua*c*a | | | aaaa*g*a | | |
| 1537 | g*a*uacaG | 3113 | — | (VPmU)*U*uau | 1539 | See Table 2. |
| | aGGUugagu | | | AcucaaccUcUg | | |
| | aua*a*a | | | uauc*u*c | | |
| 1538 | c*a*gauaG | 3114 | — | (VPmU)*G*cac | 1540 | See Table 2. |
| | gACAauuaa | | | UuaauuguCcUa | | |
| | gug*c*a | | | ucug*a*g | | |
| 1539 | g*a*uaggA | 3115 | — | (VPmU)*G*ugc | 1541 | See Table 2. |
| | cAAUuaagu | | | AcuuaauuGuCc | | |
| | gca*c*a | | | uauc*u*g | | |
| 1540 | c*c*caguU | 3116 | — | (VPmU)*G*uau | 1542 | See Table 2. |
| | uACAggucu | | | AgaccuguAaAc | | |
| | aua*c*a | | | uggg*a*a | | |
| 1541 | u*u*gagcU | 3117 | — | (VPmU)*G*ucc | 1543 | See Table 2. |
| | uACUuacuu | | | AaguaaguAaGc | | |
| | gga*c*a | | | ucaa*u*a | | |
| 1542 | a*g*cuuaC | 3118 | — | (VPmU)*U*gcg | 1544 | See Table 2. |
| | uUACuugga | | | UccaaguaAgUa | | |
| | cgc*a*a | | | agcu*c*a | | |
| 1543 | u*g*gacgC | 3119 | — | (VPmU)*C*gcu | 1545 | See Table 2. |
| | aACAuugca | | | UgcaauguUgCg | | |
| | agc*g*a | | | ucca*a*g | | |
| 1544 | g*g*acgcA | 3120 | — | (VPmU)*G*cgc | 1546 | See Table 2. |
| | aCAUugcaa | | | UugcaaugUuGc | | |
| | gcg*c*a | | | gucc*a*a | | |
| 1545 | g*a*cgcaA | 3121 | — | (VPmU)*A*gcg | 1547 | See Table 2. |
| | cAUUgcaag | | | CuugcaauGuUg | | |
| | cgc*u*a | | | cguc*c*a | | |
| 1546 | g*c*aacaU | 3122 | — | (VPmU)*C*aca | 1548 | See Table 2. |
| | uGCAagcgc | | | GcgcuugcAaUg | | |
| | ugu*g*a | | | uugc*g*u | | |
| 1547 | a*a*augaA | 3123 | — | (VPmU)*G*cga | 1549 | See Table 2. |
| | uGAUugcuu | | | AagcaaucAuUc | | |
| | ucg*c*a | | | auuu*c*u | | |
| 1548 | a*a*ugaaU | 3124 | — | (VPmU)*A*gcg | 1550 | See Table 2. |
| | gAUUgcuuu | | | AaagcaauCaUu | | |
| | cgc*u*a | | | cauu*u*c | | |
| 1549 | a*a*ugauU | 3125 | — | (VPmU)*U*aga | 1551 | See Table 2. |
| | gCUUucgcu | | | AgcgaaagCaAu | | |
| | ucu*a*a | | | cauu*c*a | | |
| 1550 | a*u*gauuG | 3126 | — | (VPmU)*G*uag | 1552 | See Table 2. |
| | cUUUcgcuu | | | AagcgaaaGcAa | | |
| | cua*c*a | | | ucau*u*c | | |
| 1551 | u*g*auugC | 3127 | — | (VPmU)*U*gua | 1553 | See Table 2. |
| | uUUCgcuuc | | | GaagcgaaAgCa | | |
| | uac*a*a | | | auca*u*u | | |
| 1552 | a*u*uuuaG | 3128 | — | (VPmU)*A*gac | 1554 | See Table 2. |
| | gGUUccauu | | | AauggaacCcUa | | |
| | guc*u*a | | | aaau*g*c | | |
| 1553 | a*u*ugucU | 3129 | — | (VPmU)*U*gua | 1555 | See Table 2. |
| | uGGUggugu | | | AcaccaccAaGa | | |
| | uac*a*a | | | caau*g*g | | |
| 1554 | u*g*guagU | 3130 | — | (VPmU)*G*cau | 1556 | See Table 2. |
| | gGAAauucu | | | AgaauuucCaCu | | |
| | aug*c*a | | | acca*u*u | | |
| 1555 | a*c*cgcuC | 3131 | — | (VPmU)*U*uua | 1557 | See Table 2. |
| | uUCAgguug | | | CaaccugaAgAg | | |
| | uaa*a*a | | | cggu*g*u | | |
| 1556 | c*a*ucuaG | 3132 | — | (VPmU)*A*uag | 1558 | See Table 2. |
| | uGUUaggca | | | UgccuaacAcUa | | |
| | cua*u*a | | | gaug*a*a | | |
| 1557 | a*u*cuagU | 3133 | — | (VPmU)*U*aua | 1559 | See Table 2. |
| | gUUAggcac | | | GugccuaaCaCu | | |
| | uau*a*a | | | agau*g*a | | |
| 1558 | g*u*uaggC | 3134 | — | (VPmU)*U*aaa | 1560 | See Table 2. |
| | aCUAuagua | | | UacuauagUgCc | | |
| | uuu*a*a | | | uaac*a*c | | |
| 1559 | u*a*aaagG | 3135 | — | (VPmU)*A*ggu | 1561 | See Table 2. |
| | uAUCaaugu | | | AcauugauAcCu | | |
| | acc*u*a | | | uuua*a*g | | |
| 1560 | c*a*ggauA | 3136 | — | (VPmU)*A*uuu | 1562 | See Table 2. |
| | aUAUagugc | | | GcacuauaUuAu | | |
| | aaa*u*a | | | ccug*c*c | | |
| 1561 | g*a*gaagC | 3137 | — | (VPmU)*C*cgc | 1563 | See Table 2. |
| | cAUAuaaug | | | CauuauauGgCu | | |
| | gcg*g*a | | | ucuc*a*u | | |
| 1562 | g*a*agccA | 3138 | — | (VPmU)*A*acc | 1564 | See Table 2. |
| | uAUAauggc | | | GccauuauAuGg | | |
| | ggu*u*a | | | cuuc*u*c | | |
| 1563 | u*g*caguA | 3139 | — | (VPmU)*U*auu | 1565 | See Table 2. |
| | cUGCaagcu | | | AgcuugcaGuAc | | |
| | aau*a*a | | | ugca*u*a | | |
| 1564 | c*u*gcaaG | 3140 | — | (VPmU)*C*caa | 1566 | See Table 2. |
| | cUAAuaacg | | | CguuauuaGcUu | | |
| | uug*g*a | | | gcag*u*a | | |
| 1565 | u*g*caagC | 3141 | — | (VPmU)*A*cca | 1567 | See Table 2. |
| | uAAUaacgu | | | AcguuauuAgCu | | |
| | ugg*u*a | | | ugca*g*u | | |
| 1566 | g*c*aagcU | 3142 | — | (VPmU)*A*acc | 1568 | See Table 2. |
| | aAUAacguu | | | AacguuauUaGc | | |
| | ggu*u*a | | | uugc*a*g | | |
| 1567 | u*a*auaaC | 3143 | — | (VPmU)*A*uaa | 1569 | See Table 2. |
| | gUUGguuug | | | CaaaccaaCgUu | | |
| | uua*u*a | | | auua*g*c | | |
| 1568 | a*g*aaagC | 3144 | — | (VPmU)*U*uga | 1570 | See Table 2. |
| | cCGCuuuag | | | CuaaagcgGgCu | | |
| | uca*a*a | | | uucu*c*u | | |
| 1569 | g*a*aagcC | 3145 | — | (VPmU)*A*uug | 1571 | See Table 2. |
| | cGCUuuagu | | | AcuaaagcGgGc | | |
| | caa*u*a | | | uuuc*u*c | | |
| 1570 | a*a*agccC | 3146 | — | (VPmU)*U*auu | 1572 | See Table 2. |
| | gCUUuaguc | | | GacuaaagCgGg | | |
| | aau*a*a | | | cuuu*c*u | | |
| 1571 | g*c*ccgcU | 3147 | — | (VPmU)*A*agu | 1573 | See Table 2. |
| | uUAGucaau | | | AuugacuaAaGc | | |
| | acu*u*a | | | gggc*u*u | | |
| 1572 | g*u*ugucU | 3148 | — | (VPmU)*A*aca | 1574 | See Table 2. |
| | gCAUuaacc | | | GguuaaugCaGa | | |
| | ugu*u*a | | | caac*u*g | | |
| 1573 | g*u*ucauA | 3149 | — | (VPmU)*G*aca | 1575 | See Table 2. |
| | cACCcauuu | | | AaauggguGuAu | | |
| | ugu*c*a | | | gaac*a*g | | |
| 1574 | u*c*auacA | 3150 | — | (VPmU)*G*gga | 1576 | See Table 2. |
| | cCCAuuuug | | | CaaaauggGuGu | | |
| | ucc*c*a | | | auga*a*c | | |

The nucleotide sequence of exemplary preferred modified versions of the dsRNA agents set forth in Table 4 are set forth in Table 5. More specifically, Table 5 sets forth the nucleotide sequence of exemplary modified sense strands, antisense strands, and dsRNA agent pairs of sense and antisense strands that may be preferred in some embodiments.

TABLE 5
Modified Sense and Antisense Strand Sequences of Exemplary Preferred
BCL11A dsRNA Agents.

| | | | | | | | Exemplary | |
|---|---|---|---|---|---|---|---|---|
| | | | | | | | mRNA | |
| | | | | | | Range | Target | |
| dsRNA | Sense | SEQ | Range in | Antisense | SEQ | in | Sequence | SEQ |
| Agent | Sequence | ID | NM_0228 | Sequence | ID | NM_ | in NM_ | ID |
| ID | 5′ to 3′ | NO | 93.4 | 5′ to 3′ | NO | 022893.4 | 022893.4 | NO |
| 770 | u*g*gauuAa | 2346 | 963-983 | (VPmU)*C*u | 772 | 961-983 | CAUGGAUUA | 3548 |
| | GAAucuaCUu | | | aaGuAGauuc | | | AGAAUCUAC | |
| | a*g*a | | | UuAa(y)cca | | | UUAGA | |
| | | | | *u*g | | | | |
| 771 | c*a*caaaCg | 2347 | 580-600 | (VPmA)*U*u | 773 | 578-600 | AGCACAAAC | 3549 |
| | GAAacaaUGc | | | gcAuUGuuuc | | | GGAAACAAU | |
| | a*a*u | | | CgUuug(y)g | | | GCAAU | |
| | | | | *c*u | | | | |
| 772 | a*c*auucUu | 2348 | 560-580 | (VPmU)*C*u | 774 | 558-580 | GGACAUUCU | 3550 |
| | AUUuuuaucg | | | cgAuaaaaau | | | UAUUUUUAU | |
| | a*g*a | | | AaGaaugu*c | | | CGAGC | |
| | | | | *C | | | | |
| 773 | g*c*aaacAg | 2349 | 908-928 | (VPmA)*A*c | 775 | 906-928 | UUGCAAACA | 3551 |
| | CCAuucaCCa | | | ugGuGAaugX | | | GCCAUUCAC | |
| | X*u*u | | | CuGu(y)ugc | | | CAGUG | |
| | | | | *a*a | | | | |
| 774 | a*u*guacAa | 2350 | 897-917 | (VPmU)*G*c | 776 | 895-917 | ACAUGUACA | 3552 |
| | CUUgcaaACa | | | ugUuUGcaag | | | ACUUGCAAA | |
| | X*c*a | | | UuGuacau*g | | | CAGCC | |
| | | | | *u | | | | |
| 775 | a*a*cacgCa | 2351 | 944-964 | (VPmA)*A*u | 777 | 942-964 | GCAACACGC | 3553 |
| | CAGaacaCUc | | | gaGuGUucug | | | ACAGAACAC | |
| | a*u*u | | | UgCguguu*g | | | UCAUG | |
| | | | | *C | | | | |
| 776 | g*c*uacaCa | 2352 | 890-910 | (VPmU)*G*c | 778 | 888-910 | CAGCUACAC | 3554 |
| | UGUacaaCUu | | | aaGuUGuaca | | | AUGUACAAC | |
| | g*c*a | | | UgUg(y)agc | | | UUGCA | |
| | | | | *u*g | | | | |
| 777 | c*a*caugUa | 2353 | 894-914 | (VPmU)*G*u | 779 | 892-914 | UACACAUGU | 3555 |
| | CAAcuugcaa | | | uuGcaaguug | | | ACAACUUGC | |
| | a*c*a | | | UaCaugug*u | | | AAACA | |
| | | | | *a | | | | |
| 778 | a*c*uugcAa | 2354 | 904-924 | (VPmU)*G*u | 780 | 902-924 | CAACUUGCA | 3556 |
| | ACAgccaUUc | | | gaAuGGcugu | | | AACAGCCAU | |
| | a*c*a | | | UuGcaag(y) | | | UCACC | |
| | | | | *u*g | | | | |
| 779 | c*g*cacaGa | 2355 | 948-968 | (VPmU)*A*u | 781 | 946-968 | CACGCACAG | 3557 |
| | ACAcucaugg | | | ccAugagugu | | | AACACUCAU | |
| | a*u*a | | | UcUgugcg*u | | | GGAUU | |
| | | | | *g | | | | |
| 780 | a*u*ucuuAu | 2356 | 562-582 | (VPmU)*U*g | 782 | 560-582 | ACAUUCUUA | 3558 |
| | UUUuaucgag | | | cuCgauaaaa | | | UUUUUAUCG | |
| | c*a*a | | | AuAagaau*g | | | AGCAC | |
| | | | | *u | | | | |
| 781 | a*g*ugcaUg | 2357 | 925-945 | (VPmU)*U*g | 783 | 923-945 | CCAGUGCAU | 3559 |
| | GUUucucUUX | | | caAgAGaaac | | | GGUUUCUCU | |
| | c*a*a | | | CaUgcac(y) | | | UGCAA | |
| | | | | *g*g | | | | |
| 782 | u*c*aucuAg | 2358 | 730-750 | (VPmU)*G*g | 784 | 728-750 | CGUCAUCUA | 3560 |
| | AGGaauuugc | | | gcAaauuccu | | | GAGGAAUUU | |
| | c*c*a | | | CuAgauga*c | | | GCCCC | |
| | | | | *g | | | | |
| 783 | g*g*cgagAa | 2359 | 2083-2103 | (VPmU)*C*c | 785 | 2081-2103 | UGGGCGAGA | 3561 |
| | GCAuaagcgc | | | gcGcuuaugc | | | AGCAUAAGC | |
| | g*g*a | | | UuCucgcc*c | | | GCGGC | |
| | | | | *a | | | | |
| 784 | g*u*aagaUg | 2360 | 2798-2818 | (VPmU)*C*a | 786 | 2796-2818 | UUGUAAGAU | 3562 |
| | CCUuuuagcg | | | cgCuaaaagg | | | GCCUUUUAG | |
| | u*g*a | | | CaUcuuac*a | | | CGUGU | |
| | | | | *a | | | | |
| 785 | g*g*gauuCa | 2361 | 1069-1089 | (VPmA)*U*u | 787 | 1067-1089 | AUGGGAUUC | 3563 |
| | UAUugcaGAc | | | guCuGCaaua | | | AUAUUGCAG | |
| | a*a*u | | | UgAa(y)ccc | | | ACAAU | |
| | | | | *a*u | | | | |
| 1575 | (NH2C6)u*g | 3151 | 963-983 | (VPmU)*C*u | 772 | 961-983 | CAUGGAUUA | 3548 |
| | *gauuAaGAA | | | aaGuAGauuc | | | AGAAUCUAC | |
| | ucuaCUua*g | | | UuAa(y)cca | | | UUAGA | |
| | *a | | | *u*g | | | | |
| 1576 | (NH2C6)c*a | 3152 | 580-600 | (VPmA)*U*u | 773 | 578-600 | AGCACAAAC | 3549 |
| | *caaaCgGAA | | | gcAuUGuuuc | | | GGAAACAAU | |
| | acaaUGca*a | | | CgUuug(y)g | | | GCAAU | |
| | *u | | | *c*u | | | | |
| 1577 | (NH2C6)a*c | 3153 | 560-580 | (VPmU)*C*u | 774 | 558-580 | GGACAUUCU | 3550 |
| | *auucUuAUU | | | cgAuaaaaau | | | UAUUUUUAU | |
| | uuuaucga*g | | | AaGaaugu*c | | | CGAGC | |
| | *a | | | *C | | | | |
| 1578 | (NH2C6)g*c | 3154 | 908-928 | (VPmA)*A*c | 775 | 906-928 | UUGCAAACA | 3551 |
| | *aaacAgCCA | | | ugGuGAaugX | | | GCCAUUCAC | |
| | uucaCCaX*u | | | CuGu(y)ugc | | | CAGUG | |
| | *u | | | *a*a | | | | |
| 1579 | (NH2C6)a*u | 3155 | 897-917 | (VPmU)*G*c | 776 | 895-917 | ACAUGUACA | 3552 |
| | *guacAaCUU | | | ugUuUGcaag | | | ACUUGCAAA | |
| | gcaaACaX*c | | | UuGuacau*g | | | CAGCC | |
| | *a | | | *u | | | | |
| 1580 | (NH2C6)a*a | 3156 | 944-964 | (VPmA)*A*u | 777 | 942-964 | GCAACACGC | 3553 |
| | *cacgCaCAG | | | gaGuGUucug | | | ACAGAACAC | |
| | aacaCUca*u | | | UgCguguu*g | | | UCAUG | |
| | *u | | | *C | | | | |
| 1581 | (NH2C6)g*c | 3157 | 890-910 | (VPmU)*G*C | 778 | 888-910 | CAGCUACAC | 3554 |
| | *uacaCaUGU | | | aaGuUGuaca | | | AUGUACAAC | |
| | acaaCUug*c | | | UgUg(y)agc | | | UUGCA | |
| | *a | | | *u*g | | | | |
| 1582 | (NH2C6)c*a | 3158 | 894-914 | (VPmU)*G*u | 779 | 892-914 | UACACAUGU | 3555 |
| | *caugUaCAA | | | uuGcaaguug | | | ACAACUUGC | |
| | cuugcaaa*c | | | UaCaugug*u | | | AAACA | |
| | *a | | | *a | | | | |
| 1583 | (NH2C6)a*c | 3159 | 904-924 | (VPmU)*G*u | 780 | 902-924 | CAACUUGCA | 3556 |
| | *uugcAaACA | | | gaAuGGcugu | | | AACAGCCAU | |
| | gccaUUca*c | | | UuGcaag(y) | | | UCACC | |
| | *a | | | *u*g | | | | |
| 1584 | (NH2C6)c*g | 3160 | 948-968 | (VPmU)*A*u | 781 | 946-968 | CACGCACAG | 3557 |
| | *cacaGaACA | | | ccAugagugu | | | AACACUCAU | |
| | cucaugga*u | | | UcUgugcg*u | | | GGAUU | |
| | *a | | | *g | | | | |
| 1585 | (NH2C6)a*u | 3161 | 562-582 | (VPmU)*U*g | 782 | 560-582 | ACAUUCUUA | 3558 |
| | *ucuuAuUUU | | | cuCgauaaaa | | | UUUUUAUCG | |
| | uaucgagc*a | | | AuAagaau*g | | | AGCAC | |
| | *a | | | *u | | | | |
| 1586 | (NH2C6)a*g | 3162 | 925-945 | (VPmU)*U*g | 783 | 923-945 | CCAGUGCAU | 3559 |
| | *ugcaUgGUU | | | caAgAGaaac | | | GGUUUCUCU | |
| | ucucUUXc*a | | | CaUgcac(y) | | | UGCAA | |
| | *a | | | *g*g | | | | |
| 1587 | (NH2C6)u*c | 3163 | 730-750 | (VPmU)*G*g | 784 | 728-750 | CGUCAUCUA | 3560 |
| | *aucuAgAGG | | | gcAaauuccu | | | GAGGAAUUU | |
| | aauuugcc*c | | | CuAgauga*c | | | GCCCC | |
| | *a | | | *g | | | | |
| 1588 | (NH2C6)g*g | 3164 | 2083-2103 | (VPmU)*C*c | 785 | 2081-2103 | UGGGCGAGA | 3561 |
| | *cgagAaGCA | | | gcGcuuaugc | | | AGCAUAAGC | |
| | uaagcgcg*g | | | UuCucgcc*c | | | GCGGC | |
| | *a | | | *a | | | | |
| 1589 | (NH2C6)g*u | 3165 | 2798-2818 | (VPmU)*C*a | 786 | 2796-2818 | UUGUAAGAU | 3562 |
| | *aagaUgCCU | | | cgCuaaaagg | | | GCCUUUUAG | |
| | uuuagcgu*g | | | CaUcuuac*a | | | CGUGU | |
| | *a | | | *a | | | | |
| 1590 | (NH2C6)g*g | 3166 | 1069-1089 | (VPmA)*U*u | 787 | 1067-1089 | AUGGGAUUC | 3563 |
| | *gauuCaUAU | | | guCuGCaaua | | | AUAUUGCAG | |
| | ugcaGAca*a | | | UgAa(y)ccc | | | ACAAU | |
| | *u | | | *a*u | | | | |

The nucleotide sequences presented in Table 4 and Table 5, utilize the following abbreviations set forth in Table 6.

TABLE 6
Abbreviations of Nucleotide Modifications
Utilized in Table 4 and Table 5.

| Abbreviation | Nucleotide/Linkage |
|---|---|
| a | 2′-O-methyladenosine |
| c | 2′-O-methylcytidine |
| g | 2′-O-methylguanosine |
| u | 2′-O-methyluridine |
| A | 2′-fluoroadenosine |
| C | 2′-fluorocytidine |
| G | 2′-fluoroguanosine |
| U | 2′-fluorouridine |
| * | Phosphorothioate Bond |
| (VPmX) | 5′-(E)-vinylphosphonate 2′-O-methylated |
| | (2′-Ome) nucleotide |
| X | Any nucleotide (A, G, C, U, T) modified or unmodified |
| Z | 2′-Fluoroguanosine unlocked nucleic acid |
| (y) | 1-N-Methylpseudouridine 2′-O-methylated (2′-Ome) |
| Nb | LNA RNA |
| Nm | 2′MOE RNA |
| dN | DNA |
| 5MCb | 5-Methylcytosine LNA RNA |
| 5MCm | 5-Methylcytosine 2′MOE RNA |
| 5MdC | 5-Methylcytosine DNA |
| (NH2C6) | aminohexyl spacer |

Various salts, mixed salts and free acid forms of the dsRNA agents are also provided herein. In some embodiments, the dsRNA agent is in a free acid form. In some embodiments, the dsRNA agent is in a salt form. In some embodiments, the dsRNA agent is in a sodium salt form. In some embodiments, wherein the dsRNA agent is in the sodium salt form, sodium ions are present in the composition comprising the dsRNA agent as counterions for substantially all of the phosphodiester or phosphorothioate groups present in the dsRNA agent. In some embodiments, wherein the dsRNA agent is in the sodium salt form, sodium ions are present in the agent as counterions for all of the phosphodiester or phosphorothioate groups present in the dsRNA agent.

### 4.3 Antisense Oligonucleotides

Provided herein are, inter alia, antisense oligonucleotides (ASOs), useful in, inter alia, inhibiting expression of BCL11 transcription factor A (BCL11A) (e.g., human BCL11A (hBCL11A)) (e.g., within a cell, e.g., within a cell in a subject, e.g., a mammalian subject, e.g., a human subject) (e.g., through the degradation of BCL11A (e.g., hBCL11A) mRNA).

Antisense oligonucleotides inhibit expression of a target gene through various mechanisms, including, e.g., cleavage of target mRNA (e.g., through the recruitment of RNAse H), blocking the association of ribosomes or other factors (steric hindrance). Antisense oligonucleotides can also modulate gene expression through modulating mRNA splicing.

In some embodiments, the antisense oligonucleotide is an RNA molecule. In some embodiments, the antisense oligonucleotide is an DNA molecule. In some embodiments, the antisense oligonucleotide is an RNA/DNA hybrid molecule. In some embodiments, the antisense oligonucleotide is a single stranded RNA molecule. In some embodiments, the antisense oligonucleotide is a single stranded DNA molecule. In some embodiments, the antisense oligonucleotide is a single stranded RNA/DNA hybrid molecule.

### 4.3.1 Targeting Region

In some embodiments, the antisense oligonucleotides comprise a region of complementarity that comprises a nucleotide sequence that is at least partially (e.g., substantially, fully) complementary to the nucleotide sequence of a target nucleic acid molecule (e.g., a target mRNA (e.g., a BCL11A mRNA), a portion of a target mRNA (e.g., a BCL11A mRNA)). In some embodiments, the nucleotide sequence of the region of complementarity is at least substantially complementary to the nucleotide sequence of the target nucleic acid molecule (e.g., a target mRNA (e.g., a BCL11A mRNA), a portion of a target mRNA (e.g., a BCL11A mRNA)). In some embodiments, the nucleotide sequence of the region of complementarity is fully complementary to the nucleotide sequence of the target nucleic acid molecule (e.g., a target mRNA (e.g., a BCL11A mRNA), a portion of a target mRNA (e.g., a BCL11A mRNA)).

In some embodiments, the nucleotide sequence of the region of complementarity is at least 70%, 75%, 80%, 85%, 90%, 91%, 92%, 93%, 94%, 95%, 96%, 97%, 98%, 99%, or 100% complementary to the nucleotide sequence of the target nucleic acid molecule (e.g., a target mRNA (e.g., a BCL11A mRNA), a portion of a target mRNA (e.g., a BCL11A mRNA)). For example, the nucleotide sequence of the region of complementarity may be at least 70% complementary to the nucleotide sequence of the target nucleic acid molecule (e.g., a target mRNA (e.g., a BCL11A mRNA), a portion of a target mRNA (e.g., a BCL11A mRNA)). The nucleotide sequence of the region of complementarity may be at least 75% complementary to the nucleotide sequence of the target nucleic acid molecule (e.g., a target mRNA (e.g., a BCL11A mRNA), a portion of a target mRNA (e.g., a BCL11A mRNA)). The nucleotide sequence of the region of complementarity may be at least 80% complementary to the nucleotide sequence of the target nucleic acid molecule (e.g., a target mRNA (e.g., a BCL11A mRNA), a portion of a target mRNA (e.g., a BCL11A mRNA)). The nucleotide sequence of the region of complementarity may be at least 85% complementary to the nucleotide sequence of the target nucleic acid molecule (e.g., a target mRNA (e.g., a BCL11A mRNA), a portion of a target mRNA (e.g., a BCL11A mRNA)). The nucleotide sequence of the region of complementarity may be at least 90% complementary to the nucleotide sequence of the target nucleic acid molecule (e.g., a target mRNA (e.g., a BCL11A mRNA), a portion of a target mRNA (e.g., a BCL11A mRNA)). The nucleotide sequence of the region of complementarity may be at least 95% complementary to the nucleotide sequence of the target nucleic acid molecule (e.g., a target mRNA (e.g., a BCL11A mRNA), a portion of a target mRNA (e.g., a BCL11A mRNA)). In some embodiments, the nucleotide sequence of the region of complementarity is at least 80%, 85%, 90%, 91%, 92%, 93%, 94%, 95%, 96%, 97%, 98%, 99%, or 100% complementary to the nucleotide sequence of the target nucleic acid molecule (e.g., a target mRNA (e.g., a BCL11A mRNA), a portion of a target mRNA (e.g., a BCL11A mRNA)). In some embodiments, the nucleotide sequence of the region of complementarity is at least 95%, 96%, 97%, 98%, 99%, or 100% (e.g., in some embodiments, preferably at least 95%, more preferably at least 98%) complementary to the nucleotide sequence of the target nucleic acid molecule (e.g., a target mRNA (e.g., a BCL11A mRNA), a portion of a target mRNA (e.g., a BCL11A mRNA)). In some embodiments, the nucleotide sequence of the region of complementarity is 100% complementary to the nucleotide sequence of the target nucleic acid molecule (e.g., a target mRNA (e.g., a BCL11A mRNA), a portion of a target mRNA (e.g., a BCL11A mRNA)).

In some embodiments, the nucleotide sequence of the region of complementarity comprises or consists of one or more non-complementary nucleotide mismatches relative to the nucleotide sequence of the target nucleic acid molecule (e.g., a target mRNA (e.g., a BCL11A mRNA), a portion of a target mRNA (e.g., a BCL11A mRNA)). In some embodiments, the nucleotide sequence of the region of complementarity comprises or consists of no more than 5 (e.g., 4, 3, 2, 1, or 0) non-complementary nucleotide mismatches relative to the nucleotide sequence of the target nucleic acid molecule. In some embodiments, the nucleotide sequence of the region of complementarity comprises or consists of no more than 3 (e.g., 2, 1, or 0) non-complementary nucleotide mismatches relative to the nucleotide sequence of the target nucleic acid molecule. In some embodiments, the nucleotide sequence of the region of complementarity comprises or consists of no more than 2 (e.g., 1 or 0) non-complementary nucleotide mismatches relative to the nucleotide sequence of the target nucleic acid molecule. In some embodiments, the nucleotide sequence of the region of complementarity comprises or consists of no more than 1 (e.g., 0) non-complementary nucleotide mismatch relative to the nucleotide sequence of the target nucleic acid molecule. In some embodiments, the nucleotide sequence of the region of complementarity comprises 0 non-complementary nucleotide mismatches relative to the nucleotide sequence of the target nucleic acid molecule. In some embodiments, the region of complementarity comprises one or more (e.g., 2, 3, or more) non-complementary nucleotide mismatches relative to the nucleotide sequence of the target nucleic acid molecule, wherein the one or more non-complementary nucleotide mismatches are within the last 5 (e.g., 4, 3, 2, or 1) nucleotides from either the 5′- and/or 3′-end of the region of complementarity. In some embodiments, the region of complementarity comprises at least one but not more than 3 non-complementary nucleotide mismatches relative to the nucleotide sequence of the target nucleic acid molecule, wherein the one or more non-complementary nucleotide mismatches are within the last 5 (e.g., 4, 3, 2, or 1) nucleotides from either the 5′- and/or 3′-end of the region of complementarity. In some embodiments, the region of complementarity comprises one or more (e.g., 2, 3, or more) non-complementary nucleotide mismatches relative to the nucleotide sequence of the target nucleic acid molecule, wherein the one or more non-complementary nucleotide mismatches are within the last 3 (e.g., 2 or 1) nucleotides from either the 5′- and/or 3′-end of the region of complementarity. In some embodiments, the region of complementarity comprises at least one but not more than 3 non-complementary nucleotide mismatches relative to the nucleotide sequence of the target nucleic acid molecule, wherein the one or more non-complementary nucleotide mismatches are within the last 3 (e.g., 2 or 1) nucleotides from either the 5′- and/or 3′-end of the region of complementarity. Methods known in the art and described herein can be utilized to evaluate the effect of any non-complementary mismatches between an antisense oligonucleotide and a target nucleic acid molecule on functional properties (e.g., inhibition of expression of the target nucleic acid molecule (e.g., a target mRNA (e.g., a BCL11A mRNA), a portion of a target mRNA (e.g., a BCL11A mRNA))).

In some embodiments, the region of complementarity comprises or consists of from about 10-30 nucleotides, e.g., 15-29, 15-28, 15-27, 15-26, 15-25, 15-24, 15-23, 15-22, 15-21, 15-20, 15-19, 15-18, 15-17, 15-16, 16-29, 16-28, 16-27, 16-26, 16-25, 16-24, 16-23, 16-22, 16-21, 16-20, 16-19, 16-18, 16-17, 17-29, 17-28, 17-27, 17-26, 17-25, 17-24, 17-23, 17-22, 17-21, 17-20, 17-19, 17-18, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, nucleotides. In some embodiments, the region of complementarity comprises from about 15-25, 15-14, 15-23, 15-22, 15-21, 15-20, 15-19, 15-18, 15-17, 15-16, 16-25, 16-14, 16-23, 16-22, 16-21, 16-20, 16-19, 16-18, 16-17, 17-25, 17-14, 17-23, 17-22, 17-21, 17-20, 17-19, 17-18, 18-25, 18-14, 18-23, 18-22, 18-21, 18-20, 18-19, 19-25, 19-14, 19-23, 19-22, 19-21, or 19-20 nucleotides. In some embodiments, the region of complementarity comprises from about 19-21 (e.g., 19-20) nucleotides. In some embodiments, the region of complementarity comprises or consists of about 15, 16, 17, 18, 19, 20, 21, 22, 23, 24, 25, 26, 27, 28, 29, or 30 nucleotides. In some embodiments, the region of complementarity comprises or consists of about 15, 16, 17, 18, 19, 20, 21, 22, 23, 24, or 25 nucleotides. In some embodiments, the region of complementarity comprises or consists of about 15, 16, 17, 18, 19, or 20 nucleotides. In some embodiments, the region of complementarity comprises or consists of about 15, 16, 17, 18, 19, 20, 21, 22, 23, 24, 25, 26, 27, 28, 29, or 30 nucleotides. In some embodiments, the region of complementarity comprises or consists of about 15, 16, 17, 18, 19, or 20 nucleotides. In some embodiments, the region of complementarity consists of about 15, 16, 17, 18, 19, or 20 nucleotides. In some embodiments, the region of complementarity consists of about 15 nucleotides. In some embodiments, the region of complementarity consists of about 16 nucleotides. In some embodiments, the region of complementarity consists of about 17 nucleotides. In some embodiments, the region of complementarity consists of about 18 nucleotides. In some embodiments, the region of complementarity consists of about 19 nucleotides. In some embodiments, the region of complementarity consists of about 20 nucleotides. Ranges and lengths intermediate to the above recited ranges and lengths are also contemplated to be part of the disclosure.

In some embodiments, the target nucleic acid molecule is part (e.g., a contiguous portion) of a larger nucleic acid molecule. For example, in some embodiments, the target nucleic acid molecule is a portion (e.g., a contiguous portion) of a target mRNA (e.g., a BCL11A mRNA). In some embodiments, the target nucleic acid molecule is a contiguous nucleotide sequence of a target mRNA (e.g., a BCL11A mRNA) of sufficient length to allow it to be a substrate for cleavage directed by an RNAi agent (e.g., an RNAi agent described herein, e.g., a dsRNA agent (e.g., described herein)) (i.e., cleavage through a RISC pathway).

In some embodiments, the target nucleic acid molecule is a target mRNA (e.g., a BCL11A mRNA). In some embodiments, the target nucleic acid molecule is at least a portion (e.g., a portion) of a target mRNA (e.g., a BCL11A mRNA). In some embodiments, the target nucleic acid molecule is at least a portion (e.g., a portion) of an mRNA (e.g., a BCL11A mRNA) formed in the expression of a target gene (e.g., a mammalian, primate, human, non-human primate, mouse, and/or rat gene) (e.g., a BCL11A gene). In some embodiments, the target nucleic acid molecule is at least a portion (e.g., a portion) of a BCL11A (e.g., hBCL11A) mRNA. In some embodiments, the target nucleic acid molecule is at least a portion (e.g., a portion) of an mRNA formed in the expression of a BCL11A (e.g., hBCL11A) gene. In some embodiments, the target nucleic acid molecule comprises at least a portion (e.g., a portion) of the nucleotide sequence set forth in SEQ ID NO: 1 (or a variant or fragment thereof). In some embodiments, the target nucleic acid molecule comprises at least a portion (e.g., a portion) of an mRNA encoding a target protein. In some embodiments, the target nucleic acid molecule comprises at least a portion (e.g., a portion) of an mRNA encoding a BCL11A (e.g., hBCL11A) protein. In some embodiments, the target nucleic acid molecule comprises at least a portion (e.g., a portion) of an mRNA sequence encoding a protein comprising the amino acid sequence set forth in SEQ ID NO: 3 (or a variant or fragment thereof).

In some embodiments, the target nucleic acid molecule comprises or consists of from about 10-30 nucleotides, e.g., 15-29, 15-28, 15-27, 15-26, 15-25, 15-24, 15-23, 15-22, 15-21, 15-20, 15-19, 15-18, 15-17, 15-16, 16-29, 16-28, 16-27, 16-26, 16-25, 16-24, 16-23, 16-22, 16-21, 16-20, 16-19, 16-18, 16-17, 17-29, 17-28, 17-27, 17-26, 17-25, 17-24, 17-23, 17-22, 17-21, 17-20, 17-19, 17-18, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, nucleotides. In some embodiments, the target nucleic acid molecule comprises from about 15-25, 15-14, 15-23, 15-22, 15-21, 15-20, 15-19, 15-18, 15-17, 15-16, 16-25, 16-14, 16-23, 16-22, 16-21, 16-20, 16-19, 16-18, 16-17, 17-25, 17-14, 17-23, 17-22, 17-21, 17-20, 17-19, 17-18, 18-25, 18-14, 18-23, 18-22, 18-21, 18-20, 18-19, 19-25, 19-14, 19-23, 19-22, 19-21, or 19-20 nucleotides. In some embodiments, the target nucleic acid molecule comprises from about 19-21 (e.g., 19-20) nucleotides. In some embodiments, the target nucleic acid molecule comprises or consists of about 15, 16, 17, 18, 19, 20, 21, 22, 23, 24, 25, 26, 27, 28, 29, or 30 nucleotides. In some embodiments, the target nucleic acid molecule comprises or consists of about 15, 16, 17, 18, 19, 20, 21, 22, 23, 24, or 25 nucleotides. In some embodiments, the target nucleic acid molecule comprises or consists of about 15, 16, 17, 18, 19, or 20 nucleotides. In some embodiments, the target nucleic acid molecule comprises or consists of about 15, 16, 17, 18, 19, 20, 21, 22, 23, 24, 25, 26, 27, 28, 29, or 30 nucleotides. In some embodiments, the target nucleic acid molecule comprises or consists of about 15, 16, 17, 18, 19, or 20 nucleotides. In some embodiments, the target nucleic acid molecule consists of about 15, 16, 17, 18, 19, or 20 nucleotides. In some embodiments, the target nucleic acid molecule consists of about 15 nucleotides. In some embodiments, the target nucleic acid molecule consists of about 16 nucleotides. In some embodiments, the target nucleic acid molecule consists of about 17 nucleotides. In some embodiments, the target nucleic acid molecule consists of about 18 nucleotides. In some embodiments, the target nucleic acid molecule consists of about 19 nucleotides. In some embodiments, the target nucleic acid molecule consists of about 20 nucleotides.

### 4.3.2 Overall Length

In some embodiments, the antisense oligonucleotide comprises or consists of from about 10-30 nucleotides, e.g., 15-29, 15-28, 15-27, 15-26, 15-25, 15-24, 15-23, 15-22, 15-21, 15-20, 15-19, 15-18, 15-17, 15-16, 16-29, 16-28, 16-27, 16-26, 16-25, 16-24, 16-23, 16-22, 16-21, 16-20, 16-19, 16-18, 16-17, 17-29, 17-28, 17-27, 17-26, 17-25, 17-24, 17-23, 17-22, 17-21, 17-20, 17-19, 17-18, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, or 19-20, nucleotides. In some embodiments, the antisense oligonucleotide comprises from about 15-25, 15-14, 15-23, 15-22, 15-21, 15-20, 15-19, 15-18, 15-17, 15-16, 16-25, 16-14, 16-23, 16-22, 16-21, 16-20, 16-19, 16-18, 16-17, 17-25, 17-14, 17-23, 17-22, 17-21, 17-20, 17-19, 17-18, 18-25, 18-14, 18-23, 18-22, 18-21, 18-20, 18-19, 19-25, 19-14, 19-23, 19-22, 19-21, or 19-20 nucleotides. In some embodiments, the antisense oligonucleotide comprises or consists of about 15, 16, 17, 18, 19, 20, 21, 22, 23, 24, 25, 26, 27, 28, 29, or 30 nucleotides. In some embodiments, the antisense oligonucleotide comprises or consists of about 15, 16, 17, 18, 19, 20, 21, 22, 23, 24, or 25 nucleotides. In some embodiments, the antisense oligonucleotide comprises or consists of about 15, 16, 17, 18, 19, or 20 nucleotides. In some embodiments, the antisense oligonucleotide comprises or consists of about 15, 16, 17, 18, 19, 20, 21, 22, 23, 24, 25, 26, 27, 28, 29, or 30 nucleotides. In some embodiments, the antisense oligonucleotide comprises or consists of about 15, 16, 17, 18, 19, or 20 nucleotides. In some embodiments, the antisense oligonucleotide consists of about 15, 16, 17, 18, 19, or 20 nucleotides. In some embodiments, the antisense oligonucleotide consists of about 15 nucleotides. In some embodiments, the antisense oligonucleotide consists of about 16 nucleotides. In some embodiments, the antisense oligonucleotide consists of about 17 nucleotides. In some embodiments, the antisense oligonucleotide consists of about 18 nucleotides. In some embodiments, the antisense oligonucleotide consists of about 19 nucleotides. In some embodiments, the antisense oligonucleotide consists of about 20 nucleotides. Ranges and lengths intermediate to the above recited ranges and lengths are also contemplated to be part of the disclosure.

### 4.3.3 Exemplary Antisense Oligonucleotides

In some embodiments, the antisense oligonucleotide comprises at least 15 (e.g., 15, 16, 17, 18, 19, 20, 21, 22, 23, 24, 25, 26, 27, 28, 29, 30 (e.g., 15, 16, 17, 18, 19, or 20)) contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide comprises at least 16 (e.g., 16, 17, 18, 19, or 20) contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide comprises at least 17 (e.g., 17, 18, 19, or 20) contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide comprises at least 20 (e.g., 20) contiguous nucleotides differing by no more than 3 (e.g., 0, 1, 2, or 3) nucleotides from the nucleotide sequence of any one of the antisense oligonucleotides set forth in Table 7 or 9 (e.g., Table 9).

In some embodiments, the antisense oligonucleotide comprises from about 10-30 nucleotides, e.g., 15-29, 15-28, 15-27, 15-26, 15-25, 15-24, 15-23, 15-22, 15-21, 15-20, 15-19, 15-18, 15-17, 15-16, 16-29, 16-28, 16-27, 16-26, 16-25, 16-24, 16-23, 16-22, 16-21, 16-20, 16-19, 16-18, 16-17, 17-29, 17-28, 17-27, 17-26, 17-25, 17-24, 17-23, 17-22, 17-21, 17-20, 17-19, 17-18, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, or 19-20 contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide comprises from about 15-25, 15-14, 15-23, 15-22, 15-21, 15-20, 15-19, 15-18, 15-17, 15-16, 16-25, 16-14, 16-23, 16-22, 16-21, 16-20, 16-19, 16-18, 16-17, 17-25, 17-14, 17-23, 17-22, 17-21, 17-20, 17-19, 17-18, 18-25, 18-14, 18-23, 18-22, 18-21, 18-20, 18-19, 19-25, 19-14, 19-23, 19-22, 19-21, or 19-20 contiguous nucleotides differing by no more than 3 (e.g., 0, 1, 2, or 3) nucleotides from the nucleotide sequence of any one of the antisense oligonucleotides set forth in Table 7 or 9 (e.g., Table 9).

In some embodiments, the nucleotide sequence of the antisense oligonucleotide comprises the nucleotide sequence of any one of the antisense oligonucleotides set forth in Table 7 or 9 (e.g., Table 9) differing by no more than 3 (e.g., 0, 1, 2, or 3) nucleotides from the antisense oligonucleotide set forth in Table 7 or 9 (e.g., Table 9). In some embodiments, the nucleotide sequence of the antisense oligonucleotide comprises the nucleotide sequence of any one of the antisense oligonucleotides set forth in Table 7 or 9 (e.g., Table 9).

In some embodiments, the antisense oligonucleotide comprises or consists of about 15, 16, 17, 18, 19, 20, 21, 22, 23, 24, 25, 26, 27, 28, 29, or 30 contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide comprises or consists of about 15, 16, 17, 18, 19, 20, 21, 22, 23, 24, or 25 contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide comprises or consists of about 15, 16, 17, 18, 19, or 20 contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide comprises or consists of about 15, 16, 17, 18, 19, 20, 21, 22, 23, 24, 25, 26, 27, 28, 29, or 30 contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide comprises or consists of about 15, 16, 17, 18, 19, or 20 contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide consists of about 15, 16, 17, 18, 19, or 20 contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide consists of about 15 contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide consists of about 16 contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide consists of about 17 contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide consists of about 18 contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide consists of about 19 contiguous nucleotides differing by no more than 3 (e.g., 0, 1, 2, or 3) nucleotides from the nucleotide sequence of any one of the antisense oligonucleotides set forth in Table 7 or 9 (e.g., Table 9). In some embodiments, the antisense oligonucleotide consists of about 20 contiguous nucleotides differing by no more than 3 (e.g., 0, 1, 2, or 3) nucleotides from the nucleotide sequence of any one of the antisense oligonucleotides set forth in Table 7 or 9 (e.g., Table 9).

In some embodiments, the antisense oligonucleotide comprises at least 15 (e.g., 15, 16, 17, 18, 19, 20, 21, 22, 23, 24, 25, 26, 27, 28, 29, 30 (e.g., 15, 16, 17, 18, 19, or 20)) contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide comprises at least 16 (e.g., 16, 17, 18, 19, or 20) contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide comprises at least 17 (e.g., 17, 18, 19, or 20) contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide comprises at least 20 (e.g., 20) contiguous nucleotides differing by no more than 3 (e.g., 0, 1, 2, or 3) nucleotides from the nucleotide sequence of any one of the antisense oligonucleotides set forth in SEQ ID NOS: 3167-3280.

In some embodiments, the antisense oligonucleotide comprises from about 10-30 nucleotides, e.g., 15-29, 15-28, 15-27, 15-26, 15-25, 15-24, 15-23, 15-22, 15-21, 15-20, 15-19, 15-18, 15-17, 15-16, 16-29, 16-28, 16-27, 16-26, 16-25, 16-24, 16-23, 16-22, 16-21, 16-20, 16-19, 16-18, 16-17, 17-29, 17-28, 17-27, 17-26, 17-25, 17-24, 17-23, 17-22, 17-21, 17-20, 17-19, 17-18, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, or 19-20 contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide comprises from about 15-25, 15-14, 15-23, 15-22, 15-21, 15-20, 15-19, 15-18, 15-17, 15-16, 16-25, 16-14, 16-23, 16-22, 16-21, 16-20, 16-19, 16-18, 16-17, 17-25, 17-14, 17-23, 17-22, 17-21, 17-20, 17-19, 17-18, 18-25, 18-14, 18-23, 18-22, 18-21, 18-20, 18-19, 19-25, 19-14, 19-23, 19-22, 19-21, or 19-20 contiguous nucleotides differing by no more than 3 (e.g., 0, 1, 2, or 3) nucleotides from the nucleotide sequence of any one of the antisense oligonucleotides set forth in SEQ ID NOS: 3167-3280.

In some embodiments, the nucleotide sequence of the antisense oligonucleotide comprises the nucleotide sequence of any one of the antisense oligonucleotides set forth in SEQ ID NOS: 3167-3280 differing by no more than 3 (e.g., 0, 1, 2, or 3) nucleotides from the antisense oligonucleotide set forth in SEQ ID NOS: 3167-3280. In some embodiments, the nucleotide sequence of the antisense oligonucleotide comprises the nucleotide sequence of any one of the antisense oligonucleotides set forth in SEQ ID NOS: 3167-3280.

In some embodiments, the antisense oligonucleotide comprises or consists of about 15, 16, 17, 18, 19, 20, 21, 22, 23, 24, 25, 26, 27, 28, 29, or 30 contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide comprises or consists of about 15, 16, 17, 18, 19, 20, 21, 22, 23, 24, or 25 contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide comprises or consists of about 15, 16, 17, 18, 19, or 20 contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide comprises or consists of about 15, 16, 17, 18, 19, 20, 21, 22, 23, 24, 25, 26, 27, 28, 29, or 30 contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide comprises or consists of about 15, 16, 17, 18, 19, or 20 contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide consists of about 15, 16, 17, 18, 19, or 20 contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide consists of about 15 contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide consists of about 16 contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide consists of about 17 contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide consists of about 18 contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide consists of about 19 contiguous nucleotides differing by no more than 3 (e.g., 0, 1, 2, or 3) nucleotides from the nucleotide sequence of any one of the antisense oligonucleotides set forth in SEQ ID NOS: 3167-3280. In some embodiments, the antisense oligonucleotide consists of about 20 contiguous nucleotides differing by no more than 3 (e.g., 0, 1, 2, or 3) nucleotides from the nucleotide sequence of any one of the antisense oligonucleotides set forth in SEQ ID NOS: 3167-3280.

In some embodiments, the nucleotide sequence of the antisense oligonucleotide comprises the nucleotide sequence set forth in any one of SEQ ID NOS: 3167-3280 differing by no more than 3 (e.g., 0, 1, 2, or 3) nucleotides from the nucleotide sequence set forth in any one of SEQ ID NOS: 3167-3280. In some embodiments, the nucleotide sequence of the antisense oligonucleotide comprises the nucleotide sequence set forth in any one of SEQ ID NOS: 3167-3280.

The disclosure further provides antisense oligonucleotides comprising at least 10-30 nucleotides, e.g., 15-29, 15-28, 15-27, 15-26, 15-25, 15-24, 15-23, 15-22, 15-21, 15-20, 15-19, 15-18, 15-17, 15-16, 16-29, 16-28, 16-27, 16-26, 16-25, 16-24, 16-23, 16-22, 16-21, 16-20, 16-19, 16-18, 16-17, 17-29, 17-28, 17-27, 17-26, 17-25, 17-24, 17-23, 17-22, 17-21, 17-20, 17-19, 17-18, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, or 19-20 contiguous nucleotides differing by no more than 3 (e.g., 0, 1, 2, or 3) nucleotides from the nucleotide sequence set forth in any one of SEQ ID NOS: 3167-3280 and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the BCL11A mRNA transcript targeted by the select antisense oligonucleotide.

In some embodiments, the antisense oligonucleotide comprises at least 15, 16, 17, 18, 19, 20, 21, 22, 23, 24, 25, 26, 27, 28, 29, or 30 contiguous nucleotides differing by no more than 3 (e.g., 0, 1, 2, or 3) nucleotides from the nucleotide sequence of any one of the antisense oligonucleotides of any one of ASOs 1-63 set forth in Table 7 or 9 (e.g., Table 9). In some embodiments, the antisense oligonucleotide comprises at least 16 (e.g., 16, 17, 18, 19, or 20) contiguous nucleotides differing by no more than 3 (e.g., 0, 1, 2, or 3) nucleotides from the nucleotide sequence of any one of the antisense oligonucleotides of any one of ASOs 1-63 set forth in Table 7 or 9 (e.g., Table 9). In some embodiments, the antisense oligonucleotide comprises at least 17 (e.g., 17, 18, 19, or 20) contiguous nucleotides differing by no more than 3 (e.g., 0, 1, 2, or 3) nucleotides from the nucleotide sequence of any one of the antisense oligonucleotides of any one of ASOs 1-63 set forth in Table 7 or 9 (e.g., Table 9). In some embodiments, the antisense oligonucleotide comprises at least 20 (e.g., 20) contiguous nucleotides differing by no more than 3 (e.g., 0, 1, 2, or 3) nucleotides from the nucleotide sequence of any one of the antisense oligonucleotides of any one of ASOs 1-63 set forth in Table 7 or 9 (e.g., Table 9).

In some embodiments, the antisense oligonucleotide comprises from about 10-30 nucleotides, e.g., 15-29, 15-28, 15-27, 15-26, 15-25, 15-24, 15-23, 15-22, 15-21, 15-20, 15-19, 15-18, 15-17, 15-16, 16-29, 16-28, 16-27, 16-26, 16-25, 16-24, 16-23, 16-22, 16-21, 16-20, 16-19, 16-18, 16-17, 17-29, 17-28, 17-27, 17-26, 17-25, 17-24, 17-23, 17-22, 17-21, 17-20, 17-19, 17-18, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, or 19-20 contiguous nucleotides differing by no more than 3 (e.g., 0, 1, 2, or 3) nucleotides from the nucleotide sequence of any one of the antisense oligonucleotides of any one of ASOs 1-63 set forth in Table 7 or 9 (e.g., Table 9). In some embodiments, the antisense oligonucleotide comprises from about 16 (e.g., 16, 17, 18, 19, or 20) contiguous nucleotides differing by no more than 3 (e.g., 0, 1, 2, or 3) nucleotides from the nucleotide sequence of any one of the antisense oligonucleotides of any one of ASOs 1-63 set forth in Table 7 or 9 (e.g., Table 9). In some embodiments, the antisense oligonucleotide comprises from about 17 (e.g., 17, 18, 19, or 20) contiguous nucleotides differing by no more than 3 (e.g., 0, 1, 2, or 3) nucleotides from the nucleotide sequence of any one of the antisense oligonucleotides of any one of ASOs 1-63 set forth in Table 7 or 9 (e.g., Table 9). In some embodiments, the antisense oligonucleotide comprises from about 20 (e.g., 17, 18, 19, or 20) contiguous nucleotides differing by no more than 3 (e.g., 0, 1, 2, or 3) nucleotides from the nucleotide sequence of any one of the antisense oligonucleotides of any one of ASOs 1-63 set forth in Table 7 or 9 (e.g., Table 9).

In some embodiments, the nucleotide sequence of the antisense oligonucleotide comprises the nucleotide sequence of any one of the antisense oligonucleotides of any one of ASOs 1-63 set forth in Table 7 or 9 (e.g., Table 9) differing by no more than 3 (e.g., 0, 1, 2, or 3) nucleotides from the antisense oligonucleotide set forth in Table 7 or 9 (e.g., Table 9). In some embodiments, the nucleotide sequence of the antisense oligonucleotide comprises the nucleotide sequence of any one of the antisense oligonucleotides of any one of ASOs 1-63 set forth in Table 7 or 9 (e.g., Table 9).

As described above, Table 7 or 9 (e.g., Table 9) further identifies the target nucleic acid molecule within the cited reference BCL11A mRNA transcript (SEQ ID NO: 1) targeted by each of the antisense oligonucleotides. The disclosure further provides antisense oligonucleotides comprising at least 15, 16, 17, 18, 19, 20, 21, 22, 23, 24, 25, 26, 27, 28, 29, or 30 contiguous nucleotides differing by no more than 3 (e.g., 0, 1, 2, or 3) nucleotides from the nucleotide sequence of any one of the antisense oligonucleotides of any one of ASOs 1-63 set forth in Table 7 or 9 (e.g., Table 9) and further comprising additional nucleotide sequences (e.g., comprising from about 1-10, 1-9, 1-8, 1-7, 1-6, 1-5, 1-4, 1-3, or 1-2 nucleotides) at least partially complementary to the region contiguous (e.g., either at the 3′ end, the 5′ end, or both the 3′ and 5′ end) of the BCL11A mRNA transcript targeted by the select antisense oligonucleotide.

It is to be understood, that although the antisense oligonucleotides set forth in Table 7 are not described as being modified (e.g., comprising chemically modified nucleotides), conjugated, etc., the disclosure includes any antisense oligonucleotide set forth in Table 7 that is unmodified, unconjugated, modified (e.g., as described herein), or conjugated (e.g., as described herein).

### 4.3.4 Antisense Oligonucleotides

The nucleotide sequence of exemplary unmodified and modified ASOs (e.g., suitable for targeting hBCL11A, suitable for inhibiting hBCL11A expression)) are set forth in Table 7.

TABLE 7
Exemplary Unmodified and Modified BCL11A Targeting ASOs.

| ASO | ASO | SEQ | | SEQ | | |
|---|---|---|---|---|---|---|
| Agent | 5′ to 3′ | ID | ASO 5′to 3′ | ID | Modification | Modification |
| ID | (Unmodified) | NO | (Modified) | NO | Pattern | Description |
| 1 | CCACCUCUGG | 3167 | InaCsmoeClnaAmo | 3218 | lQLQdddddd | Alternating terminal |
| | CACCUG | | eCdCsdTsdCsdTsd | | ddQlqL | LNA/MOE w 8 internal |
| | | | GsdGsdCsdAsmoeC | | | DNA PS + 1 PS @ 5′ and |
| | | | InaCsmoeTslnaG | | | 2 PS @ 3′ |
| 2 | CAUACCCACU | 3168 | InaCslnaAlnaTln | 3219 | lLLLdddddd | Terminal LNA w 8 |
| | UCCCUU | | aAdCsdCsdCsdAsd | | ddLllL | internal DNA PS + 1 PS |
| | | | CsdTsdTsdCslnaC | | | @ 5′ and 2 PS @ 3′ |
| | | | lnaCslnaTslnaT | | | |
| 3 | UUUCUGGGCU | 3169 | lnaTslnaTlnaTln | 3220 | lLLLdddddd | Terminal LNA w 10 |
| | GCCAUUA | | aCdTsdGsdGsdGsd | | ddddllL | internal DNA PS + 1 PS |
| | | | CsdTsdGsdCsdCsd | | | @ 5′ and 2 PS @ 3′ |
| | | | AslnaTslnaTslna | | | |
| | | | A | | | |
| 4 | AGAACAGGAA | 3170 | InaAsmoeGlnaAdA | 3221 | lQLddddddd | Alternating terminal |
| | GCAGGU | | sdCsdAsdGsdGsdA | | dddlqL | LNA/MOE w 10 internal |
| | | | sdAsdGsdCsdAsln | | | DNA PS + 1 PS @ 5′ and |
| | | | aGsmoeGslnaT | | | 2 PS @ 3′ |
| 5 | CUAGAAUUGG | 3171 | InaCslnaTlnaAln | 3222 | lLLLLddddd | Terminal LNA w 10 |
| | CCUGGGACAU | | aGlnaAdAsdTsdTs | | dddddLLllL | internal DNA PS + 1 PS |
| | | | dGsdGsdCsdCsdTs | | | @ 5′ and 2 PS @ 3′ |
| | | | dGsdGslnaGlnaAl | | | |
| | | | naCslnaAslnaT | | | |
| 6 | ACCCACUUCC | 3172 | InaAslnaClnaCln | 3223 | lLLLdddddd | Terminal LNA w 8 |
| | CUUCCU | | aCdAsdCsdTsdTsd | | ddLllL | internal DNA PS + 1 PS |
| | | | CsdCsdCsdTslnaT | | | @ 5′ and 2 PS @ 3′ |
| | | | InaCslnaCslnaT | | | |
| 7 | CCAAGCAUUG | 3173 | InaCsmoeClnaAmo | 3224 | lQLQLddddd | Alternating terminal |
| | CAUCAUCCUG | | eAlnaGdCsdAsdTs | | ddddQLQlqL | LNA/MOE w 9 internal |
| | | | dTsdGsdCsdAsdTs | | | DNA PS + 1 PS @ 5′ and |
| | | | dCsmoeAlnaTmoeC | | | 2 PS @ 3′ |
| | | | InaCsmoeTslnaG | | | |
| 8 | UCCAAGCAUU | 3174 | lnaTslnaClnaCln | 3225 | lLLLLddddd | Terminal LNA w 10 |
| | GCAUCAUCCU | | aAlnaAdGsdCsdAs | | dddddLLllL | internal DNA PS + 1 PS |
| | | | dTsdTsdGsdCsdAs | | | @ 5′ and 2 PS @ 3′ |
| | | | dTsdCslnaAlnaTl | | | |
| | | | naCslnaCsInaT | | | |
| 9 | CUCCAAGCAU | 3175 | InaCslnaTlnaCln | 3226 | lLLLLLdddd | Terminal LNA w 8 |
| | UGCAUCAUCC | | aClnaAlnaAdGsdC | | ddddLLLllL | internal DNA PS + 1 PS |
| | | | sdAsdTsdTsdGsdC | | | @ 5′ and 2 PS @ 3′ |
| | | | sdAslnaTlnaClna | | | |
| | | | AlnaTslnaCslnaC | | | |
| 10 | CCUAAGAAAC | 3176 | InaCslnaClnaTdA | 3227 | lLLddddddd | Terminal LNA w 9 |
| | AUACUG | | sdAsdGsdAsdAsdA | | ddLllL | internal DNA PS + 1 PS |
| | | | sdCsdAsdTslnaAl | | | @ 5′ and 2 PS @ 3′ |
| | | | naCslnaTslnaG | | | |
| 11 | UAUUGACCCU | 3177 | InaTsmoeAlnaImo | 3228 | lQLQLddddd | Alternating terminal |
| | GGUGUGU | | eTlnaGdAsdCsdCs | | dddQlqL | LNA/MOE w 8 internal |
| | | | dCsdTsdGsdGsdTs | | | DNA PS + 1 PS @ 5′ and |
| | | | moeGlnaTsmoeGsl | | | 2 PS @ 3′ |
| | | | naT | | | |
| 12 | UAGAAUUGGC | 3178 | lnaTsmoeAlnaGmo | 3229 | lQLQLddddd | Alternating terminal |
| | CUGGGACAU | | eAlnaAdTsdTsdGs | | dddddQlqL | LNA/MOE w 10 internal |
| | | | dGsdCsdCsdTsdGs | | | DNA PS + 1 PS @ 5′ and |
| | | | dGsdGsmoeAlnaCs | | | 2 PS @ 3′ |
| | | | moeAslnaT | | | |
| 13 | CUAGAAUUGG | 3179 | moeCsmoeTImoeAmo | 3230 | qQQQQQdddd | Terminal MOE w 8 |
| | CCUGGGACAU | | eGmoeAmoeAdTsdT | | ddddQQQqqQ | internal DNA PS + 1 PS |
| | | | sdGsdGsdCsdCsdT | | | @ 5′ and 2 PS @ 3′ |
| | | | sdGsmoeGmoeGmoe | | | |
| | | | AmoeCsmoeAsmoeT | | | |
| 14 | CUCCAAGCAU | 3180 | moeCsmoeTmoeCmo | 3231 | qQQQQddddd | Terminal MOE w 10 |
| | UGCAUCAUCC | | eCmoeAdAsdGsdCs | | dddddQQqqQ | internal DNA PS + 1 PS |
| | | | dAsdTsdTsdGsdCs | | | @ 5′ and 2 PS @ 3′ |
| | | | dAsdIsmoeCmoeAm | | | |
| | | | oeTsmoeCsmoeC | | | |
| 15 | UUGCACAUUC | 3181 | moeTsmoeTmoeGmo | 3232 | qQQQQddddd | Terminal MOE w 10 |
| | UGUAACUCCU | | eCmoeAdCsdAsdTs | | dddddQQqqQ | internal DNA PS + 1 PS |
| | | | dTsdCsdTsdGsdTs | | | @ 5′ and 2 PS @ 3′ |
| | | | dAsdAsmoeCmoeIm | | | |
| | | | oeCsmoeCsmoeT | | | |
| 16 | GGGAAAGGUU | 3182 | moeGsmoeGmoeGmo | 3233 | qQQQQddddd | Terminal MOE w 9 |
| | GCACAUUCUG | | eAmoeAdAsdGsdGs | | ddddQQQqqQ | internal DNA PS + 1 PS |
| | | | dTsdTsdGsdCsdAs | | | @ 5′ and 2 PS @ 3′ |
| | | | dCsmoeAmoeTmoeT | | | |
| | | | moeCsmoeTsmoeG | | | |
| 17 | UCGUGCCUUA | 3183 | lnaTsmoeClnaGmo | 3234 | lQLQLddddd | Alternating terminal |
| | UUCCUUCAGG | | eTlnaGdCsdCsdTs | | dddddLQlqL | LNA/MOE w 10 internal |
| | | | dTsdAsdTsdTsdCs | | | DNA PS + 1 PS @ 5′ and |
| | | | dCsdTslnaTmoeCl | | | 2 PS @ 3′ |
| | | | naAsmoeGslnaG | | | |
| 18 | CAAGCCAAAG | 3184 | lnaCsmoeAlnaAmo | 3235 | lQLQLddddd | Alternating terminal |
| | AGCUGGAGA | | eGlnaCdCsdAsdAs | | ddddLQlqL | LNA/MOE w 9 internal |
| | | | dAsdGsdAsdGsdCs | | | DNA PS + 1 PS @ 5′ and |
| | | | dTslnaGmoeGlnaA | | | 2 PS @ 3′ |
| | | | smoeGslnaA | | | |
| 19 | GGUUUCUGGG | 3185 | moeGsmoeGmoeTmo | 3236 | qQQQQddddd | Terminal MOE w 10 |
| | CUGCCAUUAU | | eTmoeTdCsdTsdGs | | dddddQQqqQ | internal DNA PS + 1 PS |
| | | | dGsdGsdCsdTsdGs | | | @ 5′ and 2 PS @ 3′ |
| | | | dCsdCsmoeAmoeTm | | | |
| | | | oeTsmoeAsmoeT | | | |
| 20 | UCUGGGAGAU | 3186 | InaTsmoeClnaImo | 3237 | lQLQLddddd | Alternating terminal |
| | CACCCUACA | | eGlnaGdGsdAsdGs | | dddddQlqL | LNA/MOE w 10 internal |
| | | | dAsdTsdCsdAsdCs | | | DNA PS + 1 PS @ 5′ and |
| | | | dCsdCsmoeTInaAs | | | 2 PS @ 3′ |
| | | | moeCslnaA | | | |
| 21 | AAAGGGAAGA | 3187 | moeAsmoeAmoeAmo | 3238 | qQQQQQdddd | Terminal MOE w 8 |
| | AUGCUACAGC | | eGmoeGmoeGdAsdA | | ddddQQQqqQ | internal DNA PS + 1 PS |
| | | | sdGsdAsdAsdTsdG | | | @ 5′ and 2 PS @ 3′ |
| | | | sdCsmoeTmoeAmoe | | | |
| | | | CmoeAsmoeGsmoeC | | | |
| 22 | CAUCCAGAAA | 3188 | lnaCsmoeAlnaImo | 3239 | lQLQLddddd | Alternating terminal |
| | CUACGUUUGU | | eClnaCdAsdGsdAs | | ddddQLQlqL | LNA/MOE w 9 internal |
| | | | dAsdAsdCsdTsdAs | | | DNA PS + 1 PS @ 5′ and |
| | | | dCsmoeGlnaTmoeT | | | 2 PS @ 3′ |
| | | | lnaTsmoeGslnaT | | | |
| 23 | UAAAUCUUGA | 3189 | lnaTsmoeAlnaAmo | 3240 | lQLQLddddd | Alternating terminal |
| | UCAAACUCAG | | eAlnaTdCsdTsdTs | | dddddLQlqL | LNA/MOE w 10 internal |
| | | | dGsdAsdTsdCsdAs | | | DNA PS + 1 PS @ 5′ and |
| | | | dAsdAslnaCmoeTl | | | 2 PS @ 3′ |
| | | | naCsmoeAslnaG | | | |
| 24 | AUCUAUAGGU | 3190 | moeAsmoeTmoeCmo | 3241 | qQQQQddddd | Terminal MOE w 10 |
| | CCAAGUUCGU | | eTmoeAdTsdAsdGs | | dddddQQqqQ | internal DNA PS + 1 PS |
| | | | dGsdTsdCsdCsdAs | | | @ 5′ and 2 PS @ 3′ |
| | | | dAsdGsmoeTmoeTm | | | |
| | | | oeCsmoeGsmoeT | | | |
| 25 | UUUAGCCACC | 3191 | moeTsmoeTmoeTmo | 3242 | qQQQQddddd | Terminal MOE w 10 |
| | UCUGGCACCU | | eAmoeGdCsdCsdAs | | dddddQQqqQ | internal DNA PS + 1 PS |
| | | | dCsdCsdTsdCsdTs | | | @ 5′ and 2 PS @ 3′ |
| | | | dGsdGsmoeCmoeAm | | | |
| | | | oeCsmoeCsmoeT | | | |
| 26 | CUAGAAUUGG | 3192 | InaCsmoeTInaAmo | 3243 | lQLQdddddd | Alternating terminal |
| | CCUGGGAC | | eGdAsdAsdTsdTsd | | ddddQlqL | LNA/MOE w 10 internal |
| | | | GsdGsdCsdCsdTsd | | | DNA PS + 1 PS @ 5′ and |
| | | | GsmoeGlnaGsmoeA | | | 2 PS @ 3′ |
| | | | slnaC | | | |
| 27 | UUGCAUCAUC | 3193 | lnaTsmoeTInaGmo | 3244 | lQLQLddddd | Alternating terminal |
| | CUGGUACCA | | eClnaAdTsdCsdAs | | dddddQlqL | LNA/MOE w 10 internal |
| | | | dTsdCsdCsdTsdGs | | | DNA PS + 1 PS @ 5′ and |
| | | | dGsdTsmoeAlnaCs | | | 2 PS @ 3′ |
| | | | moeCslnaA | | | |
| 28 | CCUCCAAGCA | 3194 | moeCsmoeCmoeTmo | 3245 | qQQQQddddd | Terminal MOE w 10 |
| | UUGCAUCAUC | | eCmoeCdAsdAsdGs | | dddddQQqqQ | internal DNA PS + 1 PS |
| | | | dCsdAsdTsdTsdGs | | | @ 5′ and 2 PS @ 3′ |
| | | | dCsdAsmoeTmoeCm | | | |
| | | | oeAsmoeTsmoeC | | | |
| 29 | GAGCUCACAG | 3195 | InaGsmoeAlnaGmo | 3246 | lQLQdddddd | Alternating terminal |
| | CCUCCAAG | | eCdTsdCsdAsdCsd | | ddddQlqL | LNA/MOE w 10 internal |
| | | | AsdGsdCsdCsdTsd | | | DNA PS + 1 PS @ 5′ and |
| | | | CsmoeClnaAsmoeA | | | 2 PS @ 3′ |
| | | | slnaG | | | |
| 30 | AACUGGAGCU | 3196 | InaAslnaAlnaCln | 3247 | lLLLLddddd | Terminal LNA w 10 |
| | UUGUGAAACC | | aTlnaGdGsdAsdGs | | dddddLLllL | internal DNA PS + 1 PS |
| | | | dCsdTsdTsdTsdGs | | | @ 5′ and 2 PS @ 3′ |
| | | | dTsdGslnaAlnaAl | | | |
| | | | naAslnaCslnaC | | | |
| 31 | CCAGGGCAGA | 3197 | moeCsmoeCmoeAmo | 3248 | qQQQQddddd | Terminal MOE w 10 |
| | ACUGGAGCUU | | eGmoeGdGsdCsdAs | | dddddQQqqQ | internal DNA PS + 1 PS |
| | | | dGsdAsdAsdCsdTs | | | @ 5′ and 2 PS @ 3′ |
| | | | dGsdGsmoeAmoeGm | | | |
| | | | oeCsmoeTsmoeT | | | |
| 32 | AAGGGAUAGC | 3198 | InaAsmoeAlnaGmo | 3249 | lQLQLddddd | Alternating terminal |
| | CCUAUGAGGC | | eGlnaGdAsdTsdAs | | dddddLQlqL | LNA/MOE w 10 internal |
| | | | dGsdCsdCsdCsdTs | | | DNA PS + 1 PS @ 5′ and |
| | | | dAsdTslnaGmoeAl | | | 2 PS @ 3′ |
| | | | naGsmoeGslnaC | | | |
| 33 | UUACAGAAUG | 3199 | lnaTslnaTlnaAln | 3250 | lLLLLddddd | Terminal LNA w 10 |
| | CCUUGUCCCA | | aClnaAdGsdAsdAs | | dddddLLllL | internal DNA PS + 1 PS |
| | | | dTsdGsdCsdCsdTs | | | @ 5′ and 2 PS @ 3′ |
| | | | dTsdGslnaTlnaCl | | | |
| | | | naCslnaCslnaA | | | |
| 34 | UAAGCAAUGG | 3200 | lnaTslnaAlnaAln | 3251 | lLLLdddddd | Terminal LNA w 10 |
| | AAGUCCAA | | aGdCsdAsdAsdTsd | | ddddLllL | internal DNA PS + 1 PS |
| | | | GsdGsdAsdAsdGsd | | | @ 5′ and 2 PS @ 3′ |
| | | | TslnaClnaCslnaA | | | |
| | | | slnaA | | | |
| 35 | CCCAGAAAAC | 3201 | InaCsmoeClnaCmo | 3252 | lQLQLQdddd | Alternating terminal |
| | CAGAACCGG | | eAlnaGmoeAdAsdA | | ddddLQlqL | LNA/MOE w 8 internal |
| | | | sdAsdCsdCsdAsdG | | | DNA PS + 1 PS @ 5′ and |
| | | | sdAslnaAmoeClna | | | 2 PS @ 3′ |
| | | | CsmoeGslnaG | | | |
| 36 | AUCCCAGAAA | 3202 | InaAslnaTlnaCln | 3253 | lLLLLddddd | Terminal LNA w 8 |
| | ACCAGAAC | | aClnaCdAsdGsdAs | | dddLLllL | internal DNA PS + 1 PS |
| | | | dAsdAsdAsdCsdCs | | | @ 5′ and 2 PS @ 3′ |
| | | | InaAlnaGlnaAsln | | | |
| | | | aAslnaC | | | |
| 37 | ACCUAAGAAA | 3203 | InaAslnaClnaCln | 3254 | lLLLdddddd | Terminal LNA w 10 |
| | CAUACUG | | aTdAsdAsdGsdAsd | | ddddllL | internal DNA PS + 1 PS |
| | | | AsdAsdCsdAsdTsd | | | @ 5′ and 2 PS @ 3′ |
| | | | AslnaCslnaTslna | | | |
| | | | G | | | |
| 38 | CUAAAUUACA | 3204 | lnaCsmoeTlnaAmo | 3255 | lQLQLddddd | Alternating terminal |
| | AGGGUGUU | | eAlnaAdTsdTsdAs | | dddLQlqL | LNA/MOE w 8 internal |
| | | | dCsdAsdAsdGsdGs | | | DNA PS + 1 PS @ 5′ and |
| | | | InaGmoeTlnaGsmo | | | 2 PS @ 3′ |
| | | | eTslnaT | | | |
| 39 | UCUAAAUUAC | 3205 | moeTsmoeCmoeTmo | 3256 | qQQQQddddd | Terminal MOE w 8 |
| | AAGGGUGU | | eAmoeAdAsdTsdTs | | dddQQqqQ | internal DNA PS + 1 PS |
| | | | dAsdCsdAsdAsdGs | | | @ 5′ and 2 PS @ 3′ |
| | | | moeGmoeGmoeTsmo | | | |
| | | | eGsmoeT | | | |
| 40 | UGUAAUUCCA | 3206 | lnaTsmoeGlnaTmo | 3257 | lQLQdddddd | Alternating terminal |
| | GGCUCUAA | | eAdAsdTsdTsdCsd | | ddddQlqL | LNA/MOE w 10 internal |
| | | | CsdAsdGsdGsdCsd | | | DNA PS + 1 PS @ 5′ and |
| | | | TsmoeClnaTsmoeA | | | 2 PS @ 3′ |
| | | | slnaA | | | |
| 41 | GUCUAGCUGC | 3207 | InaGslnaTlnaCln | 3258 | lLLLdddddd | Terminal LNA w 10 |
| | CUUCCUUA | | aTdAsdGsdCsdTsd | | ddddLllL | internal DNA PS + 1 PS |
| | | | GsdCsdCsdTsdTsd | | | @ 5′ and 2 PS @ 3′ |
| | | | CslnaClnaTslnaT | | | |
| | | | slnaA | | | |
| 42 | CCUGUCUAGC | 3208 | InaCslnaClnaTln | 3259 | lLLLdddddd | Terminal LNA w 10 |
| | UGCCUUCC | | aGdTsdCsdTsdAsd | | ddddLllL | internal DNA PS + 1 PS |
| | | | GsdCsdTsdGsdCsd | | | @ 5′ and 2 PS @ 3′ |
| | | | CslnaTlnaTslnaC | | | |
| | | | slnaC | | | |
| 43 | GCUAUCUGCU | 3209 | InaGsmoeClnaTmo | 3260 | lQLQLddddd | Alternating terminal |
| | CUUACUUAUG | | eAlnaTdCsdTsdGs | | dddddLQlqL | LNA/MOE w 10 internal |
| | | | dCsdTsdCsdTsdTs | | | DNA PS + 1 PS @ 5′ and |
| | | | dAsdCslnaTmoeTl | | | 2 PS @ 3′ |
| | | | naAsmoeTslnaG | | | |
| 44 | GCUAUCUGCU | 3210 | lnaGslnaClnaTln | 3261 | lLLLLddddd | Terminal LNA w 10 |
| | CUUACUUAU | | aAlnaTdCsdTsdGs | | dddddLllL | internal DNA PS + 1 PS |
| | | | dCsdTsdCsdTsdTs | | | @ 5′ and 2 PS @ 3′ |
| | | | dAsdCslnaTlnaTs | | | |
| | | | InaAslnaT | | | |
| 45 | UAUUACCUGU | 3211 | lnaTslnaAlnaTln | 3262 | lLLLdddddd | Terminal LNA w 9 |
| | AUGGACU | | aTdAsdCsdCsdTsd | | dddLllL | internal DNA PS + 1 PS |
| | | | GsdTsdAsdTsdGsl | | | @ 5′ and 2 PS @ 3′ |
| | | | naGlnaAslnaCsln | | | |
| | | | aT | | | |
| 46 | ACCCUGGUGU | 3212 | lnaAsmoeClnaCmo | 3263 | lQLQdddddd | Alternating terminal |
| | GUUAUGUC | | eCdTsdGsdGsdTsd | | ddddQlqL | LNA/MOE w 10 internal |
| | | | GsdTsdGsdTsdTsd | | | DNA PS + 1 PS @ 5′ and |
| | | | AsmoeTlnaGsmoeT | | | 2 PS @ 3′ |
| | | | slnaC | | | |
| 47 | UAGCUUCAAA | 3213 | lnaTsmoeAlnaGmo | 3264 | lQLQLddddd | Alternating terminal |
| | GUUGUAUUGA | | eClnaTdTsdCsdAs | | dddddLQlqL | LNA/MOE w 10 internal |
| | | | dAsdAsdGsdTsdTs | | | DNA PS + 1 PS @ 5′ and |
| | | | dGsdTslnaAmoeTl | | | 2 PS @ 3′ |
| | | | naTsmoeGslnaA | | | |
| 48 | AGAGGGAAUC | 3214 | lnaAsmoeGlnaAmo | 3265 | lQLQLddddd | Alternating terminal |
| | UGGAGAGGA | | eGlnaGdGsdAsdAs | | ddddLQlqL | LNA/MOE w 9 internal |
| | | | dTsdCsdTsdGsdGs | | | DNA PS + 1 PS @ 5′ and |
| | | | dAslnaGmoeAlnaG | | | 2 PS @ 3′ |
| | | | smoeGslnaA | | | |
| 49 | AAUUGAGAGG | 3215 | lnaAsmoeAlnaImo | 3266 | lQLQdddddd | Alternating terminal |
| | GAAUCUGG | | eTdGsdAsdGsdAsd | | ddddQlqL | LNA/MOE w 10 internal |
| | | | GsdGsdGsdAsdAsd | | | DNA PS + 1 PS @ 5′ and |
| | | | TsmoeClnaTsmoeG | | | 2 PS @ 3′ |
| | | | slnaG | | | |
| 50 | GUGGCCAGGU | 3216 | lnaGsmoeTInaGmo | 3267 | lQLQdddddd | Alternating terminal |
| | AGGACC | | eGdCsdCsdAsdGsd | | ddQlqL | LNA/MOE w 8 internal |
| | | | GsdTsdAsdGsmoeG | | | DNA PS + 1 PS @ 5′ and |
| | | | lnaAsmoeCslnaC | | | 2 PS @ 3′ |
| 51 | GUGGCCAGGU | 3217 | lnaGsmoeTInaGdG | 3268 | lQLddddddd | Alternating terminal |
| | AGGACC | | sdCsdCsdAsdGsdG | | ddQlqL | LNA/MOE w 9 internal |
| | | | sdTsdAsdGsmoeGl | | | DNA PS + 1 PS @ 5′ and |
| | | | naAsmoeCslnaC | | | 2 PS @ 3′ |

The nucleotide sequences presented in Table 7, utilize the following abbreviations set forth in Table 8.

TABLE 8
Abbreviations of Nucleotide Modifications Utilized in Table 7.

| Abbreviation | Nucleotide/Linkage |
|---|---|
| lna | Locked nucleic acid |
| s | Phosphorothioate Bond |
| moe | 2′-O-methoxyethyl |
| d | DNA nucleotide |

The nucleotide sequence of exemplary preferred unmodified and modified ASOs (e.g., suitable for targeting hBCL11A, suitable for inhibiting hBCL11A expression)) are set forth in Table 9. As described elsewhere herein, it is noted that the “T” nucleotides in the unmodified sequence may also be “U” nucleotides.

TABLE 9
Exemplary Preferred Unmodified and Modified BCL11A Targeting ASOs.

| | | |
|---|---|---|
| ASO | | |
| Agent | ASO | |
| ID | 5′ to 3′ | SEQ ID NO |

Unmodified

| | | |
|---|---|---|
| 52 | TCCGTTTGTGCTCGAT | 3269 |
| 53 | CCGTTTGTGCTCGATA | 3270 |
| 54 | CGITTGTGCTCGATAA | 3271 |
| 55 | TTCCGTTTGTGCTCGA | 3272 |

Modified

| | | |
|---|---|---|
| 56 | Tb*(5MCb)*(5MCm)*Gm*dT*dT*dT*dG*dT*dG*(5MdC)*dT*(5M | 3273 |
| | Cm)*Gm*Ab*Tb | |
| 57 | (NH2C6)Tb*(5MCb)*(5MCm)*Gm*dT*dT*dT*dG*dT*dG*(5MdC) | 3274 |
| | *dT*(5MCm)*Gm*Ab*Tb | |
| 58 | (5MCb)*(5MCb)*Gm*Tm*dT*dT*dG*dT*dG*(5MdC)*dT*(5MdC) | 3275 |
| | *Gm*Am*Tb*Ab | |
| 59 | (NH2C6)(5MCb)*(5MCb)*Gm*Tm*dT*dT*dG*dT*dG*(5MdC)*dT | 3276 |
| | *(5MdC)*Gm*Am*Tb*Ab | |
| 60 | Cb*Gb*Tm*Tm*dT*dG*dT*dG*(5MdC)*dT*(5MdC)*dG*Am*Im*A | 3277 |
| | b*Ab | |
| 61 | Cb*Cb*Gm*Tm*dT*dT*dG*dT*dG*(5MdC)*dT*(5MdC)*Gm*Am*T | 3278 |
| | b*Ab | |
| 62 | Tb*Cb*Cm*Gm*dT*dT*dT*dG*dT*dG*(5MdC)*dT*Cm*Gm*Ab*Tb | 3279 |
| 63 | Tb*Tb*Cm*Cm*dG*dT*dT*dT*dG*dT*dG*(5MdC)*Tm*Cm*Gb*Ab | 3280 |

The modified nucleotide sequences presented in Table 9, utilize the following abbreviations set forth in Table 10.

TABLE 10
Abbreviations of Nucleotide Modifications Utilized in Table 9.

| Abbreviation | Nucleotide/Linkage |
|---|---|
| a | 2′-O-methyladenosine |
| c | 2′-O-methylcytidine |
| g | 2′-O-methylguanosine |
| u | 2′-O-methyluridine |
| A | 2′-fluoroadenosine |
| C | 2′-fluorocytidine |
| G | 2′-fluoroguanosine |
| U | 2′-fluorouridine |
| * | Phosphorothioate Bond |
| VPmX | 5′-(E)-vinylphosphonate 2′-O-methylated |
| | (2′-Ome) nucleotide |
| X | Any nucleotide (A, G, C, U, T) modified or unmodified |
| Z | 2′-Fluoroguanosine unlocked nucleic acid |
| y | 1-N-Methylpseudouridine 2′-O-methylated (2′-Ome) |
| Nb | LNA RNA |
| Nm | 2′MOE RNA |
| dN | DNA |
| 5MCb | 5-Methylcytosine LNA RNA |
| 5MCm | 5-Methylcytosine 2′MOE RNA |
| 5MdC | 5-Methylcytosine DNA |
| (NH2C6) | aminohexyl spacer |

Various salts, mixed salts and free acid forms of the antisense oligonucleotides are also provided herein. In some embodiments, the antisense oligonucleotide is in a free acid form. In some embodiments, the antisense oligonucleotide is in a salt form. In some embodiments, the antisense oligonucleotide is in a sodium salt form. In some embodiments, wherein the antisense oligonucleotide is in the sodium salt form, sodium ions are present in the composition comprising the antisense oligonucleotide as counterions for substantially all of the phosphodiester or phosphorothioate groups present in the antisense oligonucleotide. In some embodiments, wherein the antisense oligonucleotide is in the sodium salt form, sodium ions are present in the agent as counterions for all of the phosphodiester or phosphorothioate groups present in the antisense oligonucleotide.

### 4.4 Modified Nucleic Acid Molecule Agents (e.g., dsRNA Agents, ASOs)

In some embodiments, a nucleic acid molecule agent described herein (or any component thereof (e.g., any nucleic acid molecule thereof)) (e.g., an antisense strand, a sense strand, a dsRNA agent, RNAi agent, an ASO, a ssRNA agent, etc.) comprises one or more modified nucleotide(s) (as defined herein). The modified agent may have one or more different (e.g., improved) properties relative to a corresponding unmodified agent. For example, the modified agent may exhibit decreased immunostimulatory activity (e.g., when administered to a subject), increased stability (e.g., in vivo, in a cell, when administered to a subject), and/or increased inhibition of expression of a target nucleic acid molecule (e.g., a target mRNA (e.g., a BCL11A mRNA)), or any combination thereof.

### 4.4.1 Nature of Nucleotide Modifications

Nucleotide modifications can include modification to any one of more of the nucleoside and/or the internucleoside linkage. Nucleoside modifications include modification to the sugar (e.g., ribose) moiety and/or the nucleobase. In some embodiments, the modified agent (or component thereof) (e.g., antisense strand, sense strand, dsRNA agent, ASO, ssRNA agent, etc.) comprises one or more nucleotides comprising a modified sugar moiety. In some embodiments, the modified agent (or component thereof) (e.g., antisense strand, sense strand, dsRNA agent, ASO, ssRNA agent, etc.) comprises one or more nucleotides comprising a modified nucleobase. In some embodiments, the modified agent (or component thereof) (e.g., antisense strand, sense strand, dsRNA agent, ASO, ssRNA agent, etc.) comprises one or more nucleotides comprising a modified internucleoside linkage. In some embodiments, the modified agent (or component thereof) (e.g., antisense strand, sense strand, dsRNA agent, ASO, ssRNA agent, etc.) comprises one or more nucleotides comprising one, two, or three of a modified sugar moiety, a modified nucleobase, and/or a modified internucleoside linkage.

Exemplary nucleotide modifications are described below and also known in the art, see, e.g., WO2021257782, WO2013075035, WO2022246251, and WO2022271573, the entire contents of each of which is incorporated by reference herein for all purposes. Exemplary modifications are further provided in Hu, B., Zhong, L., Weng, Y. et al. Therapeutic siRNA: state of the art. Sig Transduct Target Ther 5, 101 (2020). https://doi.org/10.1038/s41392-020-0207-x (e.g., Table 2), the entire contents of each of which is incorporated by reference herein for all purposes.

### 4.4.1.1 Modified Nucleosides

In some embodiments, the modified agent (or any component thereof) (e.g., antisense strand, sense strand, dsRNA agent, ASO, ssRNA agent, etc.) comprises one or more nucleotide comprising a modified nucleoside. As discussed above, nucleoside modifications can include modification of the sugar (e.g., ribose) moiety and/or modification of the nucleobase.

### (i) Sugar Modifications

In some embodiments, the modified agent (or any component thereof) (e.g., antisense strand, sense strand, dsRNA agent, ASO, ssRNA agent, etc.) comprises one or more nucleotides comprising a modified sugar (e.g., ribose, deoxyribose) moiety.

The modified sugar (e.g., ribose, deoxyribose) moiety can comprise, for example, a substituent at any one or more position of the sugar (e.g., ribose, deoxyribose), including e.g., positions 2′, 4′, and/or 5′. In some embodiments, the modified sugar (e.g., ribose, deoxyribose) comprises a substituent at 2′ position of the sugar (e.g., ribose, deoxyribose). In some embodiments, the modified sugar (e.g., ribose, deoxyribose) comprises a substituent at 5′ position of the sugar (e.g., ribose, deoxyribose). In some embodiments, the modified sugar (e.g., ribose, deoxyribose) comprises a substituent at 5′ position of the sugar (e.g., ribose, deoxyribose).

In some embodiments, the agent (or any component thereof) comprises any one or more of the following substituents (e.g., at any position of the sugar (e.g., ribose, deoxyribose) (e.g., at position 2′)): a group for improving the stability of the agent, a group for improving the pharmacokinetic properties of the agent, or a group for improving the pharmacodynamic properties of the agent, an RNA cleaving group, a reporter group, an intercalator, or other substituents having similar properties.

Exemplary substituents include, for example, but are not limited to, substitution (e.g., at any position of the sugar (e.g., ribose, deoxyribose) (e.g., at position 2′)) with any one of the following: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Additional exemplary substitutions (e.g., at any position of the sugar (e.g., ribose, deoxyribose) (e.g., at position 2′)) include, for example, but are not limited to, substitution with any one of the following: O[(CH₂)ₙO]m, CH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10.

In some embodiments, the modified sugar (e.g., ribose, deoxyribose) comprises any one of the following modifications: 2′-O-methyl (2′-OMe), 2′O-methoxyethyl (2′-O-MOE), 2′deoxy-2′-fluoro (2′-F), 2′-arabino-fluoro (2′-Ara-F), 2′-O-benzyl, 2′-O-methyl-4-pyridine (2-O-methyl-4-pyridine (2′-O-CH2Py(4)).

In some embodiments, the agent (or any component thereof) comprises any of the following substituents at the 2′-position of the sugar (e.g., ribose, deoxyribose): C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, or a substituted silyl. In some embodiments, the agent (or any component thereof) comprises a 2′-methoxyethoxy (2′-OCH₂CH₂OCH₃, also known as 2′-O-(2-methoxyethyl) or 2′-MOE) (see, e.g., Martin et al., Helv. Chim. Acta, 1995, 78:486-504, the entire contents of which is incorporated by reference herein for all purposes) (i.e., an alkoxy-alkoxy group). In some embodiments, the agent (or any component thereof) comprises a 2′-dimethylaminooxyethoxy, i.e., a O(CH₂)₂ON(CH₃)₂ group, also known as 2′-DMAOE; a 2′-dimethylaminoethoxyethoxy (also known in the art as 2′-O-dimethylaminoethoxyethyl or 2′-DMAEOE), i.e., 2′-OCH₂—O—CH₂—N(CH₃)₂; a 5′-Me-2′-F nucleotide, a 5′-Me-2′-OMe nucleotide, a 5′-Me-2′-deoxynucleotide, (both R and S isomers in these three families); a 2′-alkoxyalkyl; and 2′-NMA (N-methylacetamide).

Exemplary sugar (e.g., ribose, deoxyribose) modifications include, but are not limited to, 2′-O-methyl (2′-OMe), 2′-O-methoxyethyl (2′-O-MOE), 2′deoxy-2′-fluoro (2′-F), 2′-arabine-fluro (2′-Ara-F), 2′-O-benzyl, 2′-O-methyl-4-pyridine (2′-O-CH2Py(4)), Locked Nucleic Acid (LNA), (S)-cEt-BNA, Tricyclo-DNA (tcDNA), PMO, Unlocked Nucleic Acid (UNA), Glycol Nucleic Acid (also referred to as glycerol nucleic acid).

Exemplary US patents that describe the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920; the entire contents of each of the foregoing are hereby incorporated herein by reference for all purposes. Exemplary sugar modifications are further provided in Hu, B., Zhong, L., Weng, Y. et al. Therapeutic siRNA: state of the art. Sig Transduct Target Ther 5, 101 (2020). https://doi.org/10.1038/s41392-020-0207-x (e.g., Table 2), the entire contents of each of which is incorporated by reference herein for all purposes.

### (a) Non-Bicyclic Sugar Modifications

In some embodiments, the modified sugar (e.g., ribose, deoxyribose) moiety comprises a non-bicyclic modified sugar (e.g., ribose, deoxyribose) moiety. In some embodiments, the modified sugar (e.g., ribose, deoxyribose) moiety comprises a furanosyl ring comprising one or more substituent groups none of which bridges two atoms of the furanosyl ring to form a bicyclic structure. In some embodiments one or more non-bridging substituent of a non-bicyclic modified sugar moiety is branched. Such non bridging substituents may be at any position of the furanosyl, including but not limited to substituents at the 2′, 4′, and/or 5′ positions.

In some embodiments, non-bicyclic modified sugar moiety comprises a substituent group at the 2′-position of the sugar (e.g., ribose, deoxyribose). Examples of 2′-substituent groups suitable for non-bicyclic modified sugar moieties include but are not limited to: 2′-O-methyl (2′-OMe), 2′O-methoxyethyl (2′-O-MOE), 2′deoxy-2′-fluoro (2′-F), 2′-arabino-fluoro (2′-Ara-F), 2′-O-benzyl, 2′-O-methyl-4-pyridine (2-O-methyl-4-pyridine (2′-O-CH2Py(4)), and 2′-O—N-alkyl acetamide (e.g., 2′-O—N-methyl acetamide (“NMA”), 2′-O—N-dimethyl acetamide, 2′-O—N-ethyl acetamide, and 2′-O—N-propyl acetamide). For example, see, e.g., U.S. Pat. No. 6,147,200, Prakash et al., 2003, Org. Lett., 5, 403-6, the entire contents of which is incorporated by reference herein for all purposes.

In some embodiments, the 2′-substituent group is a halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH2)2SCH3, 0(CH2)2ON(Rm)(Rn) or OCH2C(═O)—N(Rm)(Rn), where each Rₘ and Rₙ is, independently, H, an amino protecting group, or substituted or unsubstituted C₁-C₁₀ alkyl, or a 2′-substituent group described in any one of the following: Cook et al., U.S. Pat. No. 6,531,584; Cook et al., U.S. Pat. No. 5,859,221; and Cook et al., U.S. Pat. No. 6,005,087, the entire contents of which are incorporated herein by reference for all purposes. In some embodiments, these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl.

In some embodiments, a 2′-substituted non-bicyclic modified nucleoside comprises a sugar moiety comprising a non-bridging 2′-substituent group selected from: F, NH₂, N₃, OCF₃, OCH₃, O(CH2)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl. In some embodiments, a 2′-substituted non-bicyclic modified nucleoside comprises a sugar moiety comprising a non-bridging 2′-substituent group selected from: F, OCF, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”). In some embodiments, a 2′-substituted non-bicyclic modified nucleoside comprises a sugar moiety comprising a non-bridging 2′-substituent group selected from: F, OCH₃, OCH₂CH₂OCH₃, and OCH₂C(═O)—N(H)CH₃.

In some embodiments, non-bicyclic modified sugar moiety comprises a substituent group at the 3′-position of the sugar (e.g., ribose, deoxyribose). Examples of substituent groups suitable for the 3′-position of modified sugar moieties include but are not limited to alkoxy (e.g., methoxy), alkyl (e.g., methyl, ethyl).

In some embodiments, non-bicyclic modified sugar moiety comprises a substituent group at the 4′-position of the sugar (e.g., ribose, deoxyribose). Examples of 4′-substituent groups suitable for non-bicyclic modified sugar moieties include but are not limited to alkoxy (e.g., methoxy), alkyl, and those described in Manoharan et al., WO 2015/106128.

In some embodiments, non-bicyclic modified sugar moiety comprises a substituent group at the 5′-position of the sugar (e.g., ribose, deoxyribose). Examples of substituent groups suitable for the 5′-position of modified sugar moieties include, but are not limited to, vinyl (e.g., 5′-vinyl), alkoxy (e.g., methoxy (e.g., 5′-methoxy)), and alkyl (e.g., methyl (R or S) (e.g., 5′-methyl (R or S)), ethyl).

In some embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836, the entire contents of each of which is incorporated herein by reference for all purposes.

In some embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, the entire contents of which are incorporated by reference herein for all purposes.

In some embodiments, the sugar (e.g., ribose, deoxyribose) modification comprises an unlocked nucleotide (UNA). UNA is unlocked acyclic nucleic acid, wherein any of the bonds of the sugar has been removed, forming an unlocked sugar (e.g., ribose, deoxyribose) residue. For example, in some embodiments, the bonds between C1′-C4′ have been removed (i.e., the covalent carbon-oxygen-carbon bond between the C1′ and C4′ carbons). In some embodiments, the C2′-C3′ bond (i.e., the covalent carbon-carbon bond between the C2′ and C3′ carbons) of the sugar (e.g., ribose, deoxyribose) have been removed. See, e.g., Nuc. Acids Symp. Series, 52, 133-134 (2008) and Fluiter et al., Mol. Biosyst., 2009, 10, 1039, the entire contents of which are incorporated herein by reference. UNAs and methods of making are known in the art. See, e.g., U.S. Pat. No. 8,314,227; and US2013/0096289; US2013/0011922; and US2011/0313020, the entire contents of each of which are hereby incorporated herein by reference.

### (b) Bicyclic Sugar Modifications

In some embodiments, the modified sugar (e.g., ribose, deoxyribose) moiety comprises a substituent that bridges two atoms of the furanosyl ring to form a second ring, resulting in a bicyclic sugar (e.g., ribose, deoxyribose) moiety (e.g., that locks the nucleic acid in one conformation) (locked nucleic acids (LNAs)). In some embodiments, the bicyclic sugar (e.g., ribose, deoxyribose) moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2QQ9, 74, 118-134), 4′-CH₂—C(═CH₂)-2′ and analogs thereof (see, e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672). The entire contents of all of the foregoing references is incorporated by reference herein for all purposes.

In some embodiments, such 4′ to 2′ bridges independently comprise from 1 to 4 linked groups independently selected from: —[C(Rₐ)(R_{b})]n-, —[C(Rₐ)(R_{b})]n-O—, —C(Rₐ)═C(R_{b})—, —C(Rₐ)═N—, —C(═NRₐ)—, —C(═O)—, —C(═S)—, —O—, —Si(Rₐ)₂—, —S(═O)X—, and —N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ1, NJ1J2, SJ1, N3, COOJ1, acyl (C(═O)—H), substituted acyl, CN, sulfonyl (S(=0)2-J1), or sulfoxyl (S(═O)-J1); and each J1 and J2 is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007,129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et. al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727. The entire contents of all of the foregoing references is incorporated by reference herein for all purposes.

In some embodiments, the modified sugar (e.g., ribose, deoxyribose) comprises a constrained ethyl nucleotide comprising a 4′-CH(CH₃)—O-2′ bridge. In some embodiments, the constrained ethyl nucleotide is in the S conformation (S-cEt). In some embodiments, the modified sugar (e.g., ribose, deoxyribose) comprises a conformationally restricted nucleotide (CRN). CRNs are nucleotide analogs with a linker connecting the C2′ and C4′ carbons of ribose or the C3 and —C5′ carbons of ribose. Representative publications that teach the preparation of certain of the above include, but are not limited to, US2013/0190383; and WO2013/036868, the entire contents of each of which are hereby incorporated herein by reference.

In some embodiments, bicyclic sugar moieties and nucleosides incorporating such bicyclic sugar moieties are further defined by isomeric configuration. For example, an LNA nucleoside (described herein) may be in the α-L configuration or in the β-D configuration. Herein, general descriptions of bicyclic nucleosides include both isomeric configurations. Any of the foregoing bicyclic nucleosides can be prepared having one or more stereochemical sugar configurations including for example α-L-ribofuranose and β-D-ribofuranose (see, e.g., WO 99/14226, the entire contents of which are incorporated herein by reference for all purposes).

Additional representative U.S. Patents and U.S. Patent Publications that teach the preparation of bicyclic nucleosides (e.g., locked nucleic acid) include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

### (ii) Nucleobase Modifications

In some embodiments, the modified agent (or any component thereof) (e.g., antisense strand, sense strand, dsRNA agent, ASO, ssRNA agent, etc.) comprises one or more nucleotides comprising a modified nucleobase.

As used herein, “unmodified” nucleobases refer to the purine bases adenine (A) and guanine (G), and the pyrimidine bases thymine (T), cytosine (C), and uracil (U). Modified nucleobases include other synthetic and natural nucleobases.

Exemplary nucleobase modifications, include, e.g., pseudouridine, 2′-thiouridine, N6′-methyladenosine, 5′-methylcytidine, 5′-fluoro-2′-deoxyuridine, N-ethylpiperidine 7′-EAA triazole modified adenine, N-ethylpiperidine 6′-EAA triazole modified adenine, 6′-phenylpyrrolo-cytosine, 2′,4′-difluorotoluyl ribonucleoside, and 5′-nitroindole.

Further modified nucleobases include, but are not limited to, 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 5-methylcytosine, 2-aminopropyladenine, 5-hydroxymethyl cytosine, xanthine, hypoxanthine, deoxythimidine (dT), 2-aminoadenine, 6-N-methylguanine, 6-N-methyladenine, 2-propyladenine, 2-thiouracil, 2-thiothymine and 2-thiocytosine, 5-propynyl (—C═C—CH3) uracil, 5-propynylcytosine, 6-azauracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-Nbenzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808; The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443; the entire contents of each of which is incorporated herein by reference for all purposes.

In some embodiments, the modified nucleobase comprises a pseudouridine, 2′thiouridine (s2U), N6′-methyladenosine, 5′methylcytidine (m⁵C), 5′fluoro-2′deoxyuridine, N-ethylpiperidine 7-EAA triazole modified adenine, N-ethylpiperidine 6′triazole modified adenine, 6-phenylpyrrolo-cytosine (PhpC), 2′,4′-difluorotoluyl ribonucleoside (rF), or 5′nitroindole. In some embodiments, the modified nucleobase comprises a 5-substituted pyrimidine; 6-azapyrimidine; or N-2, N-6 and 0-6 substituted purines (including 2-aminopropyladenine, 5-propynyluracil and 5-propynylcytosine). 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. Patents an published applications that teach the preparation of certain of the above noted modified nucleobases as well as other modified nucleobases include, but are not limited to, U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; 7,495,088; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121; 5,596,091; 5,614,617; 5,645,985; 5,681,941; 5,811,534; 5,750,692; 5,948,903; 5,587,470; 5,457,191; 5,763,588; 5,830,653; 5,808,027; 6,166,199; and 6,005,096, the entire contents of each of which is hereby incorporated herein by reference for all purposes. Exemplary nucleobase modifications are further provided in Hu, B., Zhong, L., Weng, Y. et al. Therapeutic siRNA: state of the art. Sig Transduct Target Ther 5, 101 (2020). https://doi.org/10.1038/s41392-020-0207-x (e.g., Table 2), the entire contents of each of which is incorporated by reference herein for all purposes.

### 4.4.1.2 Internucleoside Linkage Modifications

In some embodiments, the modified agent (or any component thereof) (e.g., antisense strand, sense strand, dsRNA agent, etc.) comprises one or more modified internucleoside linkage. Modified internucleoside linkages, compared to naturally occurring phosphate linkages, can be used to alter, typically increase, nuclease resistance of an agent (e.g., described herein).

The naturally occurring internucleoside linkage of RNA and DNA is a 3′ to 5′ phosphodiester linkage. In some embodiments, the modified internucleoside linkage contains a normal 3′-5′ linkage. In some embodiments, the modified internucleoside linkage contains a 2′-5′ linkage. In some embodiments, the modified internucleoside linkage has an inverted polarity wherein the adjacent pairs of nucleoside units are linked e.g., 3′-5′ to 5′-3′ or 2′-5′ to 5′-2′.

The two main classes of modified internucleoside linking can be defined by the presence or absence of a phosphorous atom.

Exemplary internucleoside linkage modifications are further provided in Hu, B., Zhong, L., Weng, Y. et al. Therapeutic siRNA: state of the art. Sig Transduct Target Ther 5, 101 (2020). https://doi.org/10.1038/s41392-020-0207-x (e.g., Table 2), the entire contents of each of which is incorporated by reference herein for all purposes.

### (i) Modified Phosphorous Containing Internucleoside Linkages

In some embodiments, the modified internucleoside linkage comprises a phosphorous atom. Representative modified phosphorus-containing internucleoside linkages include but are not limited to phosphorothioates (PS (Rp isomer or Sp isomer)) (e.g., 5′phosphorothioate), phosphotriesters, phosphoramidates (e.g., 3′-amino phosphoramidate and aminoalkylphosphoramidates), chiral phosphorothioates, phosphorodithioates (PS2), aminoalkylphosphotriesters, methyl and other alkyl phosphonates (e.g., methylphosphonate (MP), 3′-alkylene phosphonates), methpxypropyl-phosphonates (MOP), 5′-(E)-vinylphosphonates, 5′methyl phosphonates, (S)-5′C-methyl with phosphates, phosphinates, thionophosphoramidates, thionoalkylphosphonates, thionoalkylphosphotriesters, boranophosphates, and peptide nucleic acids (PNAs).

In some embodiments, the modified internucleoside linkage comprises phosphorothioates (PS (Rp isomer or Sp isomer)) (e.g., 5′phosphorothioate), phosphorodithioates, methylphosphonate, methoxypropyl-phosphonate, 5′-(E)-vinylphosphonate, 5′methyl phosphonate, (S)-5′C-methyl with phosphate, phosphinate, or a peptide nucleic acid.

Methods of preparing polynucleotides containing one or more modified phosphorus-containing internucleoside linkage are known in the art. See, e.g., U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference for all purposes. Exemplary modifications are further provided in Hu, B., Zhong, L., Weng, Y. et al. Therapeutic siRNA: state of the art. Sig Transduct Target Ther 5, 101 (2020). https://doi.org/10.1038/s41392-020-0207-x (e.g., Table 2), the entire contents of each of which is incorporated by reference herein for all purposes.

### (ii) Modified Non-Phosphorous Containing Internucleoside Linkages

In some embodiments, the modified internucleoside linkage does not contain a phosphorous atom. Modified internucleoside linkages that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative non-phosphorous containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—).

Methods of preparing polynucleotides comprising modified internucleoside linkages do not contain a phosphorous atom are known in the art. See, e.g., U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

### 4.4.1.3 Additional Exemplary Nucleotide Modifications

In some embodiments, the modified agent comprises one or more RNA mimetic in which both the sugar and the internucleoside linkage of the nucleotide units are replaced with novel groups. The nucleobase units are maintained for hybridization with an appropriate nucleic acid target (e.g., a target mRNA). In some embodiments, the RNA mimetic is a peptide nucleic acid (PNA). In PNAs, the ribose moiety of the RNA nucleotide is replaced with an amide containing moiety (e.g., an aminoethylglycine). The nucleobases are retained and are bound directly or indirectly to aza nitrogen atoms of the amide. Representative US patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the agents described herein are described in, for example, in Nielsen et al., Science, 1991, 254, 1497-1500, the entire contents of which is incorporated by reference herein for all purposes.

Potentially stabilizing modifications to the terminal ends of the agents (e.g., described herein) can also be incorporated to agents described herein. For example, N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Such modifications are known in the art. See, e.g., WO2011/005861, the entire contents of which is incorporated herein by reference.

### 4.4.2 Extent of Modified Nucleotides

In some embodiments, at least 5%, 10%, 15%, 20%, 25%, 30%, 35%, 40%, 45%, 50%, 55%, 60%, 65%, 70%, 75%, 80%, 85%, 90%, 95%, or 100% of the nucleotides of the agent (or any component (e.g., nucleic acid molecule) thereof) (e.g., described herein, e.g., an antisense strand, a sense strand, a dsRNA agent, agent, an ASO, an ssRNA agent) are modified. In some embodiments, about 25%, 30%, 35%, 40%, 45%, 50%, 55%, 60%, 65%, 70%, 75%, 80%, 85%, 90%, 95%, or 100% of the nucleotides of the agent (or any component (e.g., nucleic acid molecule) thereof) (e.g., described herein, e.g., an antisense strand, a sense strand, a dsRNA agent, agent, an ASO, an ssRNA agent) are modified. In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 of the nucleotides of the agent (or any component (e.g., nucleic acid molecule) thereof) (e.g., described herein, e.g., an antisense strand, a sense strand, a dsRNA agent, agent, an ASO, an ssRNA agent) are modified. In some embodiments, substantially all of the nucleotides of the agent (or any component (e.g., nucleic acid molecule) thereof) (e.g., described herein, e.g., an antisense strand, a sense strand, a dsRNA agent, agent, an ASO, an ssRNA agent) are modified. In some embodiments, all of the nucleotides of the agent (or any component (e.g., nucleic acid molecule) thereof) (e.g., described herein, e.g., an antisense strand, a sense strand, a dsRNA agent, agent, an ASO, an ssRNA agent) are modified.

In some embodiments, at least 25%, 30%, 35%, 40%, 45%, 50%, 55%, 60%, 65%, 70%, 75%, 80%, 85%, 90%, 95%, or 100% of the nucleotides of the sense and/or antisense strand are modified. For example, at least 50% of the nucleotides of the sense strand and/or antisense strand may be modified. For example, at least 55% of the nucleotides of the sense strand and/or antisense strand may be modified. For example, at least 60% of the nucleotides of the sense strand and/or antisense strand may be modified. For example, at least 65% of the nucleotides of the sense strand and/or antisense strand may be modified. For example, at least 70% of the nucleotides of the sense strand and/or antisense strand may be modified. For example, at least 75% of the nucleotides of the sense strand and/or antisense strand may be modified. For example, at least 80% of the nucleotides of the sense strand and/or antisense strand may be modified. For example, at least 85% of the nucleotides of the sense strand and/or antisense strand may be modified. For example, at least 90% of the nucleotides of the sense strand and/or antisense strand may be modified. For example, at least 90% of the nucleotides of the sense strand and/or antisense strand may be modified. For example, at least 95% of the nucleotides of the sense strand and/or antisense strand may be modified. For example, at least 96% of the nucleotides of the sense strand and/or antisense strand may be modified. For example, at least 97% of the nucleotides of the sense strand and/or antisense strand may be modified. For example, at least 98% of the nucleotides of the sense strand and/or antisense strand may be modified. For example, at least 99% of the nucleotides of the sense strand and/or antisense strand may be modified. For example, 100% of the nucleotides of the sense strand and/or antisense strand may be modified. In some embodiments, substantially all (or all) of the nucleotides in the sense strand and/or antisense strand are modified.

In some embodiments, at least 25%, 30%, 35%, 40%, 45%, 50%, 55%, 60%, 65%, 70%, 75%, 80%, 85%, 90%, 95%, or 100% of the nucleotides of the ASO are modified. For example, at least 50% of the nucleotides of the ASO may be modified. For example, at least 55% of the nucleotides of the ASO may be modified. For example, at least 60% of the nucleotides of the ASO may be modified. For example, at least 65% of the nucleotides of the ASO may be modified. For example, at least 70% of the nucleotides of the ASO may be modified. For example, at least 75% of the nucleotides of the ASO may be modified. For example, at least 80% of the nucleotides of the ASO may be modified. For example, at least 85% of the nucleotides of the ASO may be modified. For example, at least 90% of the nucleotides of the ASO may be modified. For example, at least 90% of the nucleotides of the ASO may be modified. For example, at least 95% of the nucleotides of the ASO may be modified. For example, at least 96% of the nucleotides of the ASO may be modified. For example, at least 97% of the nucleotides of the ASO may be modified. For example, at least 98% of the nucleotides of the ASO may be modified. For example, at least 99% of the nucleotides of the ASO may be modified. For example, 100% of the nucleotides of the ASO may be modified. In some embodiments, substantially all (or all) of the nucleotides in the ASO are modified.

In some embodiments, at least one of the modified nucleotides comprises a modified sugar (e.g., ribose moiety). In some embodiments, at least one of the modified nucleotides comprises a modified nucleobase. In some embodiments, the sense strand comprises at least one modified internucleoside linkage and/or the antisense strand comprises at least one modified internucleoside linkage.

In some embodiments, not more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 of the nucleotides of the of the agent (or any component (e.g., nucleic acid molecule) thereof) (e.g., described herein, e.g., an antisense strand, a sense strand, a dsRNA agent, agent, an ASO, an ssRNA agent) are unmodified. In some embodiments, not more than 10, 9, 8, 7, 6, 5, 4, 3, 2, or 1 of the nucleotides of the agent (or any component (e.g., nucleic acid molecule) thereof) (e.g., described herein, e.g., an antisense strand, a sense strand, a dsRNA agent, agent, an ASO, an ssRNA agent) are unmodified. In some embodiments, not more than 25%, 30%, 35%, 40%, 45%, 50%, 55%, 60%, 65%, 70%, 75%, 80%, 85%, 90%, 95%, or 100% of the nucleotides of the agent (or any component (e.g., nucleic acid molecule) thereof) (e.g., described herein, e.g., an antisense strand, a sense strand, a dsRNA agent, agent, an ASO, an ssRNA agent) are unmodified.

In some embodiments, at least 5%, 10%, 15%, 20%, 25%, 30%, 35%, 40%, 45%, 50%, 55%, 60%, 65%, 70%, 75%, 80%, 85%, 90%, 95%, or 100% of the nucleotides of the agent (or any component (e.g., nucleic acid molecule) thereof) (e.g., described herein, e.g., an antisense strand, a sense strand, a dsRNA agent, agent, an ASO, an ssRNA agent) are unmodified. In some embodiments, about 25%, 30%, 35%, 40%, 45%, 50%, 55%, 60%, 65%, 70%, 75%, 80%, 85%, 90%, 95%, or 100% of the nucleotides of the agent (or any component (e.g., nucleic acid molecule) thereof) (e.g., described herein, e.g., an antisense strand, a sense strand, a dsRNA agent, agent, an ASO, an ssRNA agent) are unmodified. In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 of the nucleotides of the agent (or any component (e.g., nucleic acid molecule) thereof) (e.g., described herein, e.g., an antisense strand, a sense strand, a dsRNA agent, agent, an ASO, an ssRNA agent) are unmodified. In some embodiments, substantially all of the nucleotides of the agent (or any component (e.g., nucleic acid molecule) thereof) (e.g., described herein, e.g., an antisense strand, a sense strand, a dsRNA agent, agent, an ASO, an ssRNA agent) are unmodified. In some embodiments, all of the nucleotides of the agent (or any component (e.g., nucleic acid molecule) thereof) (e.g., described herein, e.g., an antisense strand, a sense strand, a dsRNA agent, agent, an ASO, an ssRNA agent) are unmodified.

In some embodiments, not more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 of the nucleotides of the of the agent (or any component (e.g., nucleic acid molecule) thereof) (e.g., described herein, e.g., an antisense strand, a sense strand, a dsRNA agent, agent, an ASO, an ssRNA agent) are modified. In some embodiments, not more than 10, 9, 8, 7, 6, 5, 4, 3, 2, or 1 of the nucleotides of the agent (or any component (e.g., nucleic acid molecule) thereof) (e.g., described herein, e.g., an antisense strand, a sense strand, a dsRNA agent, agent, an ASO, an ssRNA agent) are modified. In some embodiments, not more than 25%, 30%, 35%, 40%, 45%, 50%, 55%, 60%, 65%, 70%, 75%, 80%, 85%, 90%, 95%, or 100% of the nucleotides of the agent (or any component (e.g., nucleic acid molecule) thereof) (e.g., described herein, e.g., an antisense strand, a sense strand, a dsRNA agent, agent, an ASO, an ssRNA agent) are modified.

In some embodiments, the agent (e.g., antisense strand, sense strand, dsRNA agent (e.g., described herein)) comprises one or more non-naturally internucleoside linkage. In some embodiments, at least 25%, 30%, 35%, 40%, 45%, 50%, 55%, 60%, 65%, 70%, 75%, 80%, 85%, 90%, 95%, or 100% of the internucleoside linkages of the agent (e.g., antisense strand, sense strand, dsRNA agent, agent, ASO, ssRNA agent, etc. (e.g., described herein)) are non-naturally occurring. In some embodiments, at least 25%, 30%, 35%, 40%, 45%, 50%, 55%, 60%, 65%, 70%, 75%, 80%, 85%, 90%, 95%, or 100% of the or internucleoside linkages of the agent (e.g., antisense strand, sense strand, dsRNA agent, agent, ASO, ssRNA agent, etc. (e.g., described herein)) are chemically modified. For example, in some embodiments, at least 50% of the or internucleoside linkages of the agent (e.g., antisense strand, sense strand, dsRNA agent, agent, ASO, ssRNA agent, etc. (e.g., described herein)) are chemically modified. For example, in some embodiments, at least 60% of the or internucleoside linkages of the agent (e.g., antisense strand, sense strand, dsRNA agent, agent, ASO, ssRNA agent, etc. (e.g., described herein)) are chemically modified. For example, in some embodiments, at least 70% of the or internucleoside linkages of the agent (e.g., antisense strand, sense strand, dsRNA agent, agent, ASO, ssRNA agent, etc. (e.g., described herein)) are chemically modified. For example, in some embodiments, at least 80% of the or internucleoside linkages of the agent (e.g., antisense strand, sense strand, dsRNA agent, agent, ASO, ssRNA agent, etc. (e.g., described herein)) are chemically modified. For example, in some embodiments, at least 90% of the or internucleoside linkages of the agent (e.g., antisense strand, sense strand, dsRNA agent, agent, ASO, ssRNA agent, etc. (e.g., described herein)) are chemically modified. For example, in some embodiments, at least 95% of the or internucleoside linkages of the agent (e.g., antisense strand, sense strand, dsRNA agent, agent, ASO, ssRNA agent, etc. (e.g., described herein)) are chemically modified. For example, in some embodiments, at least 96% of the or internucleoside linkages of the agent (e.g., antisense strand, sense strand, dsRNA agent, agent, ASO, ssRNA agent, etc. (e.g., described herein)) are chemically modified. For example, in some embodiments, at least 97% of the or internucleoside linkages of the agent (e.g., antisense strand, sense strand, dsRNA agent, agent, ASO, ssRNA agent, etc. (e.g., described herein)) are chemically modified. For example, in some embodiments, at least 98% of the or internucleoside linkages of the agent (e.g., antisense strand, sense strand, dsRNA agent, agent, ASO, ssRNA agent, etc. (e.g., described herein)) are chemically modified. For example, in some embodiments, at least 99% of the or internucleoside linkages of the agent (e.g., antisense strand, sense strand, dsRNA agent, agent, ASO, ssRNA agent, etc. (e.g., described herein)) are chemically modified. For example, in some embodiments, at least 100% of the or internucleoside linkages of the agent (e.g., antisense strand, sense strand, dsRNA agent, agent, ASO, ssRNA agent, etc. (e.g., described herein)) are chemically modified. For example, in some embodiments, all (or substantially all) of the or internucleoside linkages of the agent (e.g., antisense strand, sense strand, dsRNA agent, agent, ASO, ssRNA agent, etc. (e.g., described herein)) are chemically modified.

### 4.5 Conjugates

In some embodiments, the agent (e.g., described herein) (e.g., antisense strand, sense strand, dsRNA agent, RNAi agent, ASO, ssRNA agent, etc.) comprises a heterologous moiety (e.g., operably connected to the agent). Therefore, further provided herein are conjugates comprising an agent (e.g., described herein) (e.g., antisense strand, sense strand, dsRNA agent, RNAi agent, ASO, ssRNA agent, etc.) and a heterologous moiety (e.g., operably connected to the agent). It is clear from the disclosure, but for the sake of clarity, the conjugate can comprise a modified agent (e.g., described herein, see, e.g., § 4.4).

In some embodiments, the heterologous moiety modifies one or more property of the agent (e.g., described herein) (e.g., antisense strand, sense strand, dsRNA agent, RNAi agent, ASO, ssRNA agent, etc.) to which it is conjugated. Exemplary properties include, but are not limited to, pharmacodynamics, pharmacokinetics, stability, absorption, activity, tissue distribution, cellular distribution, cellular uptake, charge, half-life, clearance, and binding affinity to a target nucleic acid molecule (e.g., a target mRNA).

In some embodiments, the heterologous moiety enhances the distribution and/or uptake (e.g., into a cell, e.g., into a cell in a subject) of the agent (e.g., described herein) (e.g., antisense strand, sense strand, dsRNA agent, RNAi agent, ASO, ssRNA agent, etc.) (e.g., as compared to an agent that lacks the heterologous moiety). In some embodiments, the heterologous moiety alters (e.g., extends) the lifetime (e.g., in vivo) of the agent (e.g., described herein) (e.g., antisense strand, sense strand, dsRNA agent, RNAi agent, ASO, ssRNA agent, etc.) (e.g., as compared to an agent that lacks the heterologous moiety). In some embodiments, the heterologous moiety provides an enhanced affinity for a selected target, e.g., a selected molecule, cell type, compartment (e.g., cell type, tissue, organ or region of the body) (e.g., as compared to an agent that lacks the heterologous moiety).

In some embodiments, the heterologous moiety enhances the activity (e.g., in a cell, e.g., in a cell in a subject) of the agent (e.g., described herein) (e.g., antisense strand, sense strand, dsRNA agent, RNAi agent, ASO, ssRNA agent, etc.) (e.g., as compared to an agent that lacks the heterologous moiety). Activity can include, e.g., degradation of a target mRNA (e.g., a BCL11A mRNA), inhibition of expression of a target gene (e.g., a BCL11A gene), and/or reduction in the expression of a target gene (e.g., a BCL11A gene).

In some embodiments, the heterologous moiety imparts a new property on the agent (e.g., described herein) (e.g., antisense strand, sense strand, dsRNA agent, RNAi agent, ASO, ssRNA agent, etc.) to which it is conjugated. For example, fluorophores or reporter groups that enable detection of the agent (e.g., described herein).

It is to be understood the heterologous moieties can impart multiple (e.g., any combination of the foregoing) properties of the agent (e.g., described herein) (e.g., antisense strand, sense strand, dsRNA agent, RNAi agent, ASO, ssRNA agent, etc.).

In some embodiments, wherein the agent is a dsRNA agent comprising a double stranded region, the heterologous moiety does not take part in, does not alter, and/or does not interfere with, the creation of a double strand region.

### 4.5.1 Heterologous Moieties

Heterologous moieties, include for example, but are not limited to, carbohydrates, peptides, proteins (e.g., antibodies or functional fragments or variants thereof; ligands (e.g., of a target receptor)), lipids, polymers, small molecules, intercalators, reporter molecules, polyamines, polyamides, vitamin moieties, polyethylene glycols, thioethers, polyethers, cholesterols, thiocholesterols, cholic acid moieties, folate, lipophilic groups, phospholipids, biotin, phenazine, phenanthridine, anthraquinone, adamantane, acridine, fluoresceins, rhodamines, coumarins, fluorophores, and dyes.

In some embodiments, the heterologous moiety is a carbohydrate, peptide, protein (e.g., antibody or functional fragment or variant thereof, e.g., ligand (e.g., of a target receptor)), lipid, polymer, small molecule, or any combination thereof. In some embodiments, the heterologous moiety comprises an active drug substance. In some embodiments, the heterologous moiety does not contain an active drug substance.

Exemplary heterologous moieties (e.g., targeting moieties), further include but are not limited, to carbohydrate moieties (e.g., GalNAc and GalNAc derivatives (See, e.g., U.S. Pat. No. 8,106,022 and WO2019055633)); lipid moieties such as a cholesterol moiety (see, e.g., Letsinger et al., Proc. Natl. Acid. Sci. USA, 1989, 86: 6553-6556); cholic acid (see, e.g., Manoharan et al., Biorg. Med. Chem. Let., 1994, 4:1053-1060), a thioether, e.g., beryl-S-tritylthiol (see, e.g., Manoharan et al., Ann. N.Y. Acad. Sci., 1992, 660:306-309; Manoharan et al., Biorg. Med. Chem. Let., 1993, 3:2765-2770); thiocholesterols (see, e.g., Oberhauser et al., Nucl. Acids Res., 1992, 20:533-538); aliphatic chains (e.g., dodecandiol or undecyl residues) (see, e.g., Saison-Behmoaras et al., EMBO J, 1991, 10:1111-1118; Kabanov et al., FEBS Lett., 1990, 259:327-330; Svinarchuk et al., Biochimie, 1993, 75:49-54), phospholipids (e.g., dihexadecyl-rac-glycerol or triethyl-ammonium 1,2-di-O-hexadecyl-rac-glycero-3-phosphonate) (see, e.g., Manoharan et al., Tetrahedron Lett., 1995, 36:3651-3654; Shea et al., Nucl. Acids Res., 1990, 18:3777-3783); polyamine or polyethylene glycol chains (see, e.g., Manoharan et al., Nucleosides & Nucleotides, 1995, 14:969-973); adamantane acetic acids (see, e.g., Manoharan et al., Tetrahedron Lett., 1995, 36:3651-3654); palmityl moieties (see, e.g., Mishra et al., Biochim. Biophys. Acta, 1995, 1264:229-237); and octadecylamine or hexylamino-carbonyloxycholesterol moiety (see, e.g., Crooke et al., J. Pharmacol. Exp. Ther., 1996, 277:923-937). The entire contents of each of the foregoing references is incorporated herein by reference for all purposes. Additional carbohydrate heterologous moieties (and linkers) suitable for use in conjugates described herein include those described in PCT Publication Nos. WO 2014/179620 and WO 2014/179627, the entire contents of each of which are incorporated herein by reference for all purposes.

### 4.5.1.1 Targeting Moieties

In specific embodiments, the heterologous moiety is a targeting moiety. In some embodiments, the targeting moiety enhances distribution of the agent (e.g., described herein) (e.g., antisense strand, sense strand, dsRNA agent, RNAi agent, ASO, ssRNA agent, etc.) to a target cell (or population of cells), tissue, and/or organ (e.g., as compared to an agent that lacks the targeting moiety). In some embodiments, the targeting moiety enhances the uptake of the agent (e.g., described herein) (e.g., antisense strand, sense strand, dsRNA agent, RNAi agent, ASO, ssRNA agent, etc.) into a target cell (or population of cells) (e.g., as compared to an agent that lacks the targeting moiety). In some embodiments, the targeting moiety provides enhanced affinity for a selected target, e.g., molecule, cell, cell type, compartment, e.g., a cellular or organ compartment, tissue, organ or region of the body, (e.g., as compared to an agent that lacks the targeting moiety).

In some embodiments, the targeting moiety specifically binds to a target molecule (e.g., protein, carbohydrate, lipid, etc.) expressed on the surface of a target cell, tissue, and/or organ. In some embodiments, the target molecule is a protein, carbohydrate, or lipid. In some embodiments, the target molecule is a receptor.

In some embodiments, upon binding to TFR (e.g., hTFR (e.g., TFR1)) expressed on the surface of a hematopoietic cell, the conjugate is internalized into the hematopoietic cell.

In some embodiments, the conjugate exhibits one or more of the following properties: (a) upon binding to TFR (e.g., hTFR (e.g., TFR1)) expressed on the surface of a hematopoietic cell, the conjugate does not induce death of the target cell; (b) upon binding to TFR (e.g., hTFR (e.g., TFR1)) expressed on the surface of a hematopoietic cell, the hematopoietic cell remains viable; (c) upon internalization into a hematopoietic cell, the conjugate does not induce death of the hematopoietic cell; and/or (d) upon binding to TFR (e.g., hTFR (e.g., TFR1)) expressed on the surface of a hematopoietic cell, the conjugate does not induce degradation of TFR (e.g., hTFR (e.g., TFR1)).

In some embodiments, upon binding to TFR (e.g., hTFR (e.g., TFR1)) expressed on the surface of an erythroid precursor cell, the conjugate is internalized into the erythroid precursor cell.

In some embodiments, the conjugate exhibits one or more of the following properties: (a) upon binding to TFR (e.g., hTFR (e.g., TFR1)) expressed on the surface of an erythroid precursor cell, the conjugate does not induce death of the target cell; (b) upon binding to TFR (e.g., hTFR (e.g., TFR1)) expressed on the surface of an erythroid precursor cell, the erythroid precursor cell remains viable; (c) upon internalization into an erythroid precursor cell, the conjugate does not induce death of the erythroid precursor cell; and/or (d) upon binding to TFR (e.g., hTFR (e.g., TFR1)) expressed on the surface of an erythroid precursor cell, the conjugate does not induce degradation of TFR (e.g., hTFR (e.g., TFR1)).

In some embodiments, the conjugate exhibits one or more of the following properties: (a) upon binding to TFR (e.g., hTFR (e.g., TFR1)) expressed on the surface of an erythroid precursor cell, the conjugate is internalized into the erythroid precursor cell; (b) upon binding to TFR (e.g., hTFR (e.g., TFR1)) expressed on the surface of an erythroid precursor cell, the conjugate does not induce death of the target cell; (c) upon binding to TFR (e.g., hTFR (e.g., TFR1)) expressed on the surface of an erythroid precursor cell, the erythroid precursor cell remains viable; (d) upon internalization into an erythroid precursor cell, the conjugate does not induce death of the erythroid precursor cell; and/or (e) upon binding to TFR (e.g., hTFR (e.g., TFR1)) expressed on the surface of an erythroid precursor cell, the conjugate does not induce degradation of TFR (e.g., hTFR (e.g., TFR1)).

In some embodiments, the protein (e.g., an antibody) that specifically binds to the TFR (e.g., hTFR (e.g., hTFR1)) exhibits one or more of the following properties: (a) upon binding to TFR (e.g., hTFR (e.g., TFR1)) expressed on the surface of an erythroid precursor cell, the protein (e.g., an antibody) that specifically binds to the TFR (e.g., hTFR (e.g., hTFR1)) or conjugate is internalized into the erythroid precursor cell; (b) upon binding to TFR (e.g., hTFR (e.g., TFR1)) expressed on the surface of an erythroid precursor cell, the protein (e.g., an antibody) that specifically binds to the TFR (e.g., hTFR (e.g., hTFR1)) or conjugate does not induce death of the target cell; (c) upon binding to TFR (e.g., hTFR (e.g., TFR1)) expressed on the surface of an erythroid precursor cell, the erythroid precursor cell remains viable; (d) upon internalization into an erythroid precursor cell, the protein (e.g., an antibody) that specifically binds to the TFR (e.g., hTFR (e.g., hTFR1)) or conjugate does not induce death of the erythroid precursor cell; and/or (e) upon binding to TFR (e.g., hTFR (e.g., TFR1)) expressed on the surface of an erythroid precursor cell, the protein (e.g., an antibody) that specifically binds to the TFR (e.g., hTFR (e.g., hTFR1)) or conjugate does not induce degradation of TFR (e.g., hTFR (e.g., TFR1)).

In some embodiments, the conjugates described herein exhibit one or more of the following properties: (a) upon binding to a target molecule (e.g., TFR (e.g., hTFR (e.g., hTFR1))) expressed on the surface of a target cell (e.g., an erythroid precursor cell), the conjugate is internalized into the target cell; (b) upon binding to a target molecule (e.g., TFR (e.g., hTFR (e.g., hTFR1))) expressed on the surface of a target cell (e.g., an erythroid precursor cell), the conjugate does not induce death of the target cell; (c) upon binding to a target molecule (e.g., TFR (e.g., hTFR (e.g., hTFR1))) expressed on the surface of a target cell (e.g., an erythroid precursor cell), the target cell remains viable; (d) upon internalization into a target cell (e.g., an erythroid precursor cell), the conjugate does not induce death of the target cell; and/or (e) upon binding to a target molecule (e.g., TFR (e.g., hTFR (e.g., hTFR1))) expressed on the surface of a target cell (e.g., an erythroid precursor cell), the conjugate does not induce degradation of the target molecule (e.g., TFR (e.g., hTFR (e.g., hTFR1))).

### (i) Hematopoietic Cell Targeting Moieties

In some embodiments, the targeting moiety is a hematopoietic cell (e.g., an erythroid precursor cell) targeting moiety. In some embodiments, the targeting moiety is capable of targeting an agent described herein (e.g., an antisense strand, sense strand, dsRNA agent, RNAi agent, ASO, ssRNA agent, etc. described herein) to a hematopoietic cell (e.g., an erythroid precursor cell). In some embodiments, the hematopoietic cell (e.g., an erythroid precursor cell) is present in the bone marrow (e.g., of a subject). In some embodiments, the targeting moiety is capable of targeting an agent (e.g., an antisense strand, sense strand, dsRNA agent, RNAi agent, ASO, ssRNA agent, etc. described herein) to one or more cell within the bone marrow. In some embodiments the targeting moiety specifically targets erythroid precursor cells (e.g., within the bone marrow (e.g., within a subject)). In some embodiments the targeting moiety specifically targets erythroid precursor cells (e.g., within the bone marrow (e.g., within a subject)) through specific binding to an antigen expressed on the surface of the erythroid precursor cells (e.g., TFR (e.g., hTFR (e.g., hTFR1))).

It should be appreciated that various types of targeting moieties (e.g., hematopoietic cell (e.g., erythroid precursor cell) targeting moieties) may be used in accordance with the disclosure. For example, the targeting moiety (e.g., the hematopoietic cell (e.g., erythroid precursor cell) targeting moiety) may comprise (or consist of) a small molecule, an oligonucleotide (e.g., DNA, RNA, RNA/DNA hybrid) (e.g., an aptamer), a protein (e.g., an antibody, a peptide), a lipid (e.g., a microvesicle), or a carbohydrate (e.g., a polysaccharide). In some embodiments, the targeting moiety is a protein. In some embodiments, the targeting moiety is a peptide. In some embodiments, the targeting moiety is an antibody. In some embodiments, the targeting moiety is an antibody-like scaffold (e.g., as described herein). Exemplary targeting moieties (e.g., hematopoietic cell (e.g., erythroid precursor cell) targeting moieties) are described in further detail herein, however, it should be appreciated that the exemplary targeting moieties (e.g., hematopoietic cell (e.g., erythroid precursor cell) targeting moieties) provided herein are not meant to be limiting.

In some embodiments, the hematopoietic cell (e.g., erythroid precursor cell) targeting moiety specifically binds to an antigen expressed on surface of hematopoietic cells (or one or more subset thereof) (e.g., erythroid precursor cells) (e.g., within the bone marrow) (e.g., TFR (e.g., hTFR (e.g., hTFR1))). In some embodiments, the hematopoietic cell (e.g., erythroid precursor cell) targeting moiety specifically binds to an antigen expressed on the surface of erythroid precursor cells (e.g., TFR (e.g., hTFR (e.g., hTFR1))). In some embodiments, the hematopoietic cell (e.g., erythroid precursor cell) targeting moiety specifically binds to an antigen expressed on the surface of erythroid precursor cells within the bone marrow (e.g., TFR (e.g., hTFR (e.g., hTFR1))).

By interacting with one or more molecule (e.g., protein) expressed on the surface of a target hematopoietic cell (e.g., erythroid precursor cell) both tissue (e.g., bone marrow) localization and selective or preferred uptake into hematopoietic cells (e.g., erythroid precursor cells) can be achieved. In some embodiments, molecules (e.g., proteins) that are substrates for hematopoietic cell (e.g., erythroid precursor cell) uptake transporters are useful for delivering an agent (e.g., an antisense strand, sense strand, dsRNA agent, RNAi agent, ASO, ssRNA agent, etc. described herein) into hematopoietic cells (e.g., erythroid precursor cells). Binding to molecules (e.g., proteins) expressed on the surface of hematopoietic cells (e.g., erythroid precursor cells) followed by endocytosis can allow large molecules such as antibodies to enter the hematopoietic cells (e.g., erythroid precursor cells). For example, as described in detail below, agents (e.g., an antisense strand, sense strand, dsRNA agent, RNAi agent, ASO, ssRNA agent, etc. described herein) conjugated to transferrin (or a functional fragment or functional variant thereof) or anti-TFR (e.g., hTFR (e.g., hTFR1)) antibodies can be taken up by hematopoietic cells (e.g., erythroid precursor cells) via binding to TFR (e.g., hTFR (e.g., hTFR1)), which may then be endocytosed, e.g., via endocytosis, e.g., clathrin-mediated endocytosis.

The use of hematopoietic cell (e.g., erythroid precursor cell) targeting moieties may be useful for concentrating an agent (e.g., an antisense strand, sense strand, dsRNA agent, RNAi agent, ASO, ssRNA agent, etc. described herein) in hematopoietic cells (e.g., erythroid precursor cells (e.g., within the bone marrow (e.g., within a subject))) while reducing toxicity associated with effects in other cells or tissues. In some embodiments, the hematopoietic cell (e.g., erythroid precursor cell) targeting moiety concentrates a bound agent (e.g., an antisense strand, sense strand, dsRNA agent, RNAi agent, ASO, ssRNA agent, etc. described herein) in hematopoietic cells (e.g., erythroid precursor cell (e.g., within the bone marrow (e.g., within a subject))) as compared to another tissue or cell type within a subject. In some embodiments, the hematopoietic cell (e.g., erythroid precursor cell) targeting moiety concentrates a bound agent (e.g., an antisense strand, sense strand, dsRNA agent, RNAi agent, ASO, ssRNA agent, etc. described herein) in hematopoietic cells (e.g., erythroid precursor cells) in an amount that is at least 1, 2, 3, 4, 5, 6, 7, 8, 9, 10, 15, 20, 30, 40, 50, 60, 70, 80, 90, or 100 times greater than an amount in non-hematopoietic cells (e.g., non-erythroid precursor cells). In some embodiments, a toxicity of the agent (e.g., an antisense strand, sense strand, dsRNA agent, RNAi agent, ASO, ssRNA agent, etc. described herein) in a subject is reduced by at least 1%, 2%, 3%, 4%, 5%, 10%, 15%, 20%, 25%, 30%, 35%, 40%, 45%, 50%, 55%, 60%, 65%, 70%, 75%, 80%, 90%, or 95% when it is delivered to the subject when bound to the hematopoietic cell (e.g., erythroid precursor cell) targeting moiety.

In some embodiments, the hematopoietic cell (e.g., erythroid precursor cell) targeting moiety exhibits one or more of the following properties: (a) upon binding to a target molecule (e.g., TFR (e.g., hTFR (e.g., hTFR1))) expressed on the surface of a target cell (e.g., an erythroid precursor cell), the hematopoietic cell (e.g., erythroid precursor cell) targeting moiety is internalized into the target cell; (b) upon binding to a target molecule (e.g., TFR (e.g., hTFR (e.g., hTFR1))) expressed on the surface of a target cell (e.g., an erythroid precursor cell), the hematopoietic cell (e.g., erythroid precursor cell) targeting moiety does not induce death of the target cell; (c) upon binding to a target molecule (e.g., TFR (e.g., hTFR (e.g., hTFR1))) expressed on the surface of a target cell (e.g., an erythroid precursor cell), the target cell remains viable; (d) upon internalization into a target cell (e.g., an erythroid precursor cell), the hematopoietic cell (e.g., erythroid precursor cell) targeting moiety does not induce death of the target cell; and/or (e) upon binding to a target molecule (e.g., TFR (e.g., hTFR (e.g., hTFR1))) expressed on the surface of a target cell (e.g., an erythroid precursor cell), the hematopoietic cell (e.g., erythroid precursor cell) targeting moiety does not induce degradation of the target molecule (e.g., TFR (e.g., hTFR (e.g., hTFR1))).

### (a) TFR Targeting Moieties

In some embodiments, the targeting moiety (e.g., the hematopoietic cell (e.g., erythroid precursor cell) targeting moiety) specifically binds to the transferrin receptor (TFR) (e.g., hTFR (e.g., hTFR1)). In some embodiments, the targeting moiety (e.g., the hematopoietic cell (e.g., erythroid precursor cell) targeting moiety) specifically binds to hTFR. In some embodiments, the targeting moiety (e.g., the hematopoietic cell (e.g., erythroid precursor cell) targeting moiety) specifically binds hTFR1.

TFR1 is a transmembrane homodimeric glycoprotein that functions, inter alia, in the cellular uptake of iron from the plasma glycoprotein transferrin (TF). Iron uptake from transferrin involves the binding of TF to TFR (e.g., TFR1), internalization of TF within an endocytic vesicle by receptor-mediated endocytosis and the release of iron induced by a decrease in endosomal pH. TFR1 is expressed by e.g., placental syncytiotrophoblasts, myocytes, basal keratinocytes, hepatocytes, endocrine pancreas, spermatocytes, and erythroid precursor cells. TFR expression, while known to be highly expressed by erythroid precursor cells, is not expressed by mature erythrocytes. TFR2 is a known homolog of TFR1, but TFR1 is considered the major protein responsible for iron uptake owing to its higher affinity and expression pattern. See, e.g., Derek K. Marsee, et al., CD71 (Transferrin Receptor): An Effective Marker for Erythroid Precursors in Bone Marrow Biopsy Specimens, American Journal of Clinical Pathology, Volume 134, Issue 3, September 2010, Pages 429-435, https://doi.org/10.1309/AJCPCRK3MOAOJ6AT; C. Sieff et al., Changes in Cell Surface Antigen Expression During Hemopoietic Differentiation, Blood, Vol 60 (3), 1982, Pages 703-713, https://doi.org/10.1182/blood.V60.3.703.703; the entire contents of each of which are incorporated herein by reference for all purposes.

The amino acid sequence of a reference hTFR1 protein is set forth in SEQ ID NO: 3281. The amino acid sequence of a reference hTFR2 protein is set forth in SEQ ID NO: 3282. The amino acid sequence of a reference hTF protein is set forth in SEQ ID NO: 3283. See Table 11, herein.

TABLE 11
The Amino Acid Sequence of a Reference hTFR1, hTFR2, and hTF Protein.

| | | SEQ ID |
|---|---|---|
| Description | Amino Acid Sequence | NO |
| hTFR1 | MMDQARSAFSNLEGGEPLSYTRFSLARQVDGDNSHVEMKLAVDEEENA | 3281 |
| UniProt | DNNTKANVTKPKRCSGSICYGTIAVIVFFLIGFMIGYLGYCKGVEPKT | |
| Accession | ECERLAGTESPVREEPGEDFPAARRLYWDDLKRKLSEKLDSTDFTGTI | |
| P02786\| | KLLNENSYVPREAGSQKDENLALYVENQFREFKLSKVWRDQHFVKIQV | |
| | KDSAQNSVIIVDKNGRLVYLVENPGGYVAYSKAATVTGKLVHANFGTK | |
| | KDFEDLYTPVNGSIVIVRAGKITFAEKVANAESLNAIGVLIYMDQTKF | |
| | PIVNAELSFFGHAHLGTGDPYTPGFPSFNHTQFPPSRSSGLPNIPVQT | |
| | ISRAAAEKLFGNMEGDCPSDWKTDSTCRMVTSESKNVKLTVSNVLKEI | |
| | KILNIFGVIKGFVEPDHYVVVGAQRDAWGPGAAKSGVGTALLLKLAQM | |
| | FSDMVLKDGFQPSRSIIFASWSAGDEGSVGATEWLEGYLSSLHLKAFT | |
| | YINLDKAVLGTSNFKVSASPLLYTLIEKTMQNVKHPVTGQFLYQDSNW | |
| | ASKVEKLILDNAAFPFLAYSGIPAVSFCFCEDTDYPYLGTTMDTYKEL | |
| | IERIPELNKVARAAAEVAGQFVIKLTHDVELNLDYERYNSQLLSFVRD | |
| | LNQYRADIKEMGLSLOWLYSARGDFFRATSRLITDFGNAEKTDRFVMK | |
| | KLNDRVMRVEYHFLSPYVSPKESPFRHVFWGSGSHILPALLENLKLRK | |
| | QNNGAFNETLERNQLALATWTIQGAANALSGDVWDIDNEF | |
| hTFR2 | MERLWGLFQRAQQLSPRSSQTVYQRVEGPRKGHLEEEEEDGEEGAETL | 3282 |
| UniProt | AHFCPMELRGPEPLGSRPROPNLIPWAAAGRRAAPYLVLTALLIFTGA | |
| Accession | FLLGYVAFRGSCQACGDSVLVVSEDVNYEPDLDFHQGRLYWSDLQAMF | |
| Q9UP52 | LQFLGEGRLEDTIRQTSLRERVAGSAGMAALTQDIRAALSROKLDHVW | |
| | TDTHYVGLQFPDPAHPNTLHWVDEAGKVGEQLPLEDPDVYCPYSAIGN | |
| | VTGELVYAHYGRPEDLQDLRARGVDPVGRLLLVRVGVISFAQKVTNAQ | |
| | DFGAQGVLIYPEPADFSQDPPKPSLSSQQAVYGHVHLGTGDPYTPGFP | |
| | SFNQTQFPPVASSGLPSIPAQPISADIASRLLRKLKGPVAPQEWQGSL | |
| | LGSPYHLGPGPRLRLVVNNHRISTPINNIFGCIEGRSEPDHYVVIGAQ | |
| | RDAWGPGAAKSAVGTAILLELVRTFSSMVSNGFRPRRSLLFISWDGGD | |
| | FGSVGSTEWLEGYLSVLHLKAVVYVSLDNAVLGDDKFHAKTSPLLTSL | |
| | IESVLKQVDSPNHSGQTLYEQVVFTNPSWDAEVIRPLPMDSSAYSFTA | |
| | FVGVPAVEFSFMEDDQAYPFLHTKEDTYENLHKVLOGRLPAVAQAVAQ | |
| | LAGQLLIRLSHDRLLPLDFGRYGDVVLRHIGNLNEFSGDLKARGLTLQ | |
| | WVYSARGDYIRAAEKLRQEIYSSEERDERLTRMYNVRIMRVEFYFLSQ | |
| | YVSPADSPFRHIFMGRGDHTLGALLDHLRLLRSNSSGTPGATSSTGFQ | |
| | ESRFRRQLALLTWTLQGAANALSGDVWNIDNNF | |
| hTF | VPDKTVRWCAVSEHEATKCQSFRDHMKSVIPSDGPSVACVKKASYLDC | 3283 |
| (Mature - no | IRAIAANEADAVTLDAGLVYDAYLAPNNLKPVVAEFYGSKEDPQTFYY | |
| signal sequence) | AVAVVKKDSGFQMNQLRGKKSCHTGLGRSAGWNIPIGLLYCDLPEPRK | |
| UniProt | PLEKAVANFFSGSCAPCADGTDFPQLCQLCPGCGCSTLNQYFGYSGAF | |
| Accession | KCLKDGAGDVAFVKHSTIFENLANKADRDQYELLCLDNTRKPVDEYKD | |
| P02787 | CHLAQVPSHTVVARSMGGKEDLIWELLNQAQEHFGKDKSKEFQLFSSP | |
| | HGKDLLFKDSAHGFLKVPPRMDAKMYLGYEYVTAIRNLREGTCPEAPT | |
| | DECKPVKWCALSHHERLKCDEWSVNSVGKIECVSAETTEDCIAKIMNG | |
| | EADAMSLDGGFVYIAGKCGLVPVLAENYNKSDNCEDTPEAGYFAVAVV | |
| | KKSASDLTWDNLKGKKSCHTAVGRTAGWNIPMGLLYNKINHCRFDEFF | |
| | SEGCAPGSKKDSSLCKLCMGSGLNLCEPNNKEGYYGYTGAFRCLVEKG | |
| | DVAFVKHQTVPQNTGGKNPDPWAKNLNEKDYELLCLDGTRKPVEEYAN | |
| | CHLARAPNHAVVTRKDKEACVHKILRQQQHLFGSNVTDCSGNFCLFRS | |
| | ETKDLLFRDDTVCLAKLHDRNTYEKYLGEEYVKAVGNLRKCSTSSLLE | |
| | ACTFRRP | |

In some embodiments, the targeting moiety (e.g., the hematopoietic cell (e.g., erythroid precursor cell) targeting moiety) specifically binds TFR1. In some embodiments, the targeting moiety (e.g., the hematopoietic cell (e.g., erythroid precursor cell) targeting moiety) specifically binds TFR2. In some embodiments, the targeting moiety (e.g., the hematopoietic cell (e.g., erythroid precursor cell) targeting moiety) specifically binds TFR1 and TFR2. In some embodiments, the targeting moiety (e.g., the hematopoietic cell (e.g., erythroid precursor cell) targeting moiety) specifically binds TFR1 but does not specifically bind TFR2. In some embodiments, the targeting moiety (e.g., the hematopoietic cell (e.g., erythroid precursor cell) targeting moiety) specifically binds TFR1 and binds to TFR2 with significantly lower affinity.

In some embodiments, the targeting moiety (e.g., the hematopoietic cell (e.g., erythroid precursor cell) targeting moiety) specifically binds hTFR1 and one or more of murine TFR1, rat TFR1, a non-human primate TFR1 (e.g., cynomolgus TFR1).

In some embodiments, the TFR (e.g., hTFR (e.g., hTFR1)) targeting moiety enhances the distribution and/or uptake (e.g., into a cell, e.g., into a cell in a subject, e.g., a cell that expresses TFR (e.g., hTFR (e.g., hTFR1)) (e.g., an erythroid precursor cell (e.g., in the bone marrow))) of an agent (e.g., an antisense strand, sense strand, dsRNA agent, RNAi agent, ASO, ssRNA agent, etc. described herein) (e.g., as compared to an oligonucleotide that lacks the targeting moiety). In some embodiments, the TFR (e.g., hTFR (e.g., hTFR1)) targeting moiety alters (e.g., extends) the lifetime (e.g., in vivo) of the agent (e.g., an antisense strand, sense strand, dsRNA agent, RNAi agent, ASO, ssRNA agent, etc. described herein) (e.g., as compared to an oligonucleotide that lacks the targeting moiety). In some embodiments, the TFR (e.g., hTFR (e.g., hTFR1)) targeting moiety provides an enhanced affinity for a selected target, e.g., a selected cell type, compartment (e.g., cell type, tissue, organ or region of the body) (e.g., as compared to an oligonucleotide that lacks the targeting moiety) (e.g., erythroid precursor cells (e.g., in the bone marrow)).

In some embodiments, the TFR targeting moiety does not (or does not significantly) interfere with TF binding to TFR. In some embodiments, the TFR targeting moiety does not (or does not significantly) compete for binding to TFR with TF.

In some embodiments, the TFR targeting moiety is a protein. In some embodiments, the targeting moiety is a transferrin protein. In some embodiments, the targeting moiety is an antibody. In some embodiments, the targeting moiety comprises an antibody-like scaffold (e.g., a cysteine dense peptide, see, e.g., WO2023023031, the entire contents of which is incorporated by reference herein for all purposes).

### TF Proteins

In some embodiments, the TFR targeting moiety comprises TF (e.g., hTF) (or a functional fragment or variant thereof). In some embodiments, the TFR targeting moiety comprises the TFR1 binding domain of TF (e.g., hTF) (or a functional fragment or variant thereof). In some embodiments, the TF (e.g., hTF) (or a functional fragment or variant thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 3283.

Variant hTF proteins are known in the art, see, e.g., WO2009019314A1, WO2008152140A2, EP2216341A1, WO2009149393A2, U.S. Pat. No. 8,158,579B2, the entire contents of each of which are incorporated herein by reference for all purposes. In some embodiments, the variant hTF protein exhibits increased stability and/or long plasma half-life (e.g., relative to a reference hTF protein) that does not comprise the one or more variation). In some embodiments, the variant hTF protein comprises a substitution of a non-cysteine amino acid residue with a cysteine (e.g., at position V1, P2, D3, K4 T5, H14, Q20, S21, D24, K27, S28, V29, P31, S32, D33, A43, E89, D104, G106, G114, L122, G123, P145, S155, D163, T165, D166, P168, P175, G176, G178, C179, S180, T181, L182, Q184, F187, S189, D197, G198, E212, A215, N216, A218, D221, D229, G257, N268, D277, K278, K280, E281, S287, P288, H289, K291, S298, P307, L326, T330, P335, T336, N413, S415, D416, D420, K434, S435, A436, S437, D438, D442, N443, G446, N469, N472, G487, K489, D491, S501, G502, L503, N510, T518, P539, Q540, G543, G544, K545, P547, D548, P549, K552, N553, N555, D558, D565, T567, P570, N576, A595, S610, N611, V612, T613, D614, S616, G617, T626, D634, D643, S666, T667 or S669, numbering according to SEQ ID NO: 3283) (e.g., as described in WO2009019314A1). In some embodiments, the variant hTF protein comprises the addition of a cysteine residue (e.g., as described in WO2009019314A1).

### Exemplary hTF Variant Proteins

The amino acid sequence of exemplary hTF variants is provided in Table 12.

TABLE 12
The Amino Acid Sequence of Exemplary hTF Variants.

| | | SEQ |
|---|---|---|
| Description | Amino Acid Sequence | ID NO |
| A | CPDKTVRWCAVSEHEATKCQSFRDHMKSVIPSDGPSVACVKKASYLDCI | 3284 |
| | RAIAANEADAVTLDAGLVYDAYLAPNNLKPVVAEFYGSKEDPQTFYYAV | |
| | AVVKKDSGFQMNQLRGKKSCHTGLGRSAGWNIPIGLLYCDLPEPRKPLE | |
| | KAVANFFSGSCAPCADGTDFPQLCQLCPGCGCSTLNQYFGYSGAFKCLK | |
| | DGAGDVAFVKHSTIFENLANKADRDQYELLCLDNTRKPVDEYKDCHLAQ | |
| | VPSHTVVARSMGGKEDLIWELLNQAQEHFGKDKSKEFQLFSSPHGKDLL | |
| | FKDSAHGFLKVPPRMDAKMYLGYEYVTAIRNLREGTCPEAPTDECKPVK | |
| | WCALSHHERLKCDEWSVNSVGKIECVSAETTEDCIAKIMNGEADAMSLD | |
| | GGFVYIAGKCGLVPVLAENYNKSDNCEDTPEAGYFAVAVVKKSASDLTW | |
| | DNLKGKKSCHTAVGRTAGWNIPMGLLYNKINHCRFDEFFSEGCAPGSKK | |
| | DSSLCKLCMGSGLNLCEPNNKEGYYGYTGAFRCLVEKGDVAFVKHQTVP | |
| | QNTGGKNPDPWAKNLNEKDYELLCLDGTRKPVEEYANCHLARAPNHAVV | |
| | TRKDKEACVHKILRQQQHLFGSNVTDCSGNFCLFRSETKDLLFRDDTVC | |
| | LAKLHDRNTYEKYLGEEYVKAVGNLRKCSTSSLLEACTERRP | |
| B | VPDKTVRWCAVSEHEATKCQSFRDHMKCVIPSDGPSVACVKKASYLDCI | 3285 |
| | RAIAANEADAVTLDAGLVYDAYLAPNNLKPVVAEFYGSKEDPQTFYYAV | |
| | AVVKKDSGFQMNQLRGKKSCHTGLGRSAGWNIPIGLLYCDLPEPRKPLE | |
| | KAVANFFSGSCAPCADGTDFPQLCQLCPGCGCSTLNQYFGYSGAFKCLK | |
| | DGAGDVAFVKHSTIFENLANKADRDQYELLCLDNTRKPVDEYKDCHLAQ | |
| | VPSHTVVARSMGGKEDLIWELLNQAQEHFGKDKSKEFQLFSSPHGKDLL | |
| | FKDSAHGFLKVPPRMDAKMYLGYEYVTAIRNLREGTCPEAPTDECKPVK | |
| | WCALSHHERLKCDEWSVNSVGKIECVSAETTEDCIAKIMNGEADAMSLD | |
| | GGFVYIAGKCGLVPVLAENYNKSDNCEDTPEAGYFAVAVVKKSASDLTW | |
| | DNLKGKKSCHTAVGRTAGWNIPMGLLYNKINHCRFDEFFSEGCAPGSKK | |
| | DSSLCKLCMGSGLNLCEPNNKEGYYGYTGAFRCLVEKGDVAFVKHQTVP | |
| | QNTGGKNPDPWAKNLNEKDYELLCLDGTRKPVEEYANCHLARAPNHAVV | |
| | TRKDKEACVHKILRQQQHLFGSNVTDCSGNFCLFRSETKDLLFRDDTVC | |
| | LAKLHDRNTYEKYLGEEYVKAVGNLRKCSTSSLLEACTFRRP | |
| C | VPDKTVRWCAVSEHEATKCQSFRDHMKSVIPCDGPSVACVKKASYLDCI | 3286 |
| | RAIAANEADAVTLDAGLVYDAYLAPNNLKPVVAEFYGSKEDPQTFYYAV | |
| | AVVKKDSGFQMNQLRGKKSCHTGLGRSAGWNIPIGLLYCDLPEPRKPLE | |
| | KAVANFFSGSCAPCADGTDFPQLCQLCPGCGCSTLNQYFGYSGAFKCLK | |
| | DGAGDVAFVKHSTIFENLANKADRDQYELLCLDNTRKPVDEYKDCHLAQ | |
| | VPSHTVVARSMGGKEDLIWELLNQAQEHFGKDKSKEFQLFSSPHGKDLL | |
| | FKDSAHGFLKVPPRMDAKMYLGYEYVTAIRNLREGTCPEAPTDECKPVK | |
| | WCALSHHERLKCDEWSVNSVGKIECVSAETTEDCIAKIMNGEADAMSLD | |
| | GGFVYIAGKCGLVPVLAENYNKSDNCEDTPEAGYFAVAVVKKSASDLTW | |
| | DNLKGKKSCHTAVGRTAGWNIPMGLLYNKINHCRFDEFFSEGCAPGSKK | |
| | DSSLCKLCMGSGLNLCEPNNKEGYYGYTGAFRCLVEKGDVAFVKHQTVP | |
| | QNTGGKNPDPWAKNLNEKDYELLCLDGTRKPVEEYANCHLARAPNHAVV | |
| | TRKDKEACVHKILRQQQHLFGSNVTDCSGNFCLFRSETKDLLERDDTVC | |
| | LAKLHDRNTYEKYLGEEYVKAVGNLRKCSTSSLLEACTFRRP | |
| D | VPDKTVRWCAVSEHEATKCQSFRDHMKSVIPSDGPSVACVKKASYLDCI | 3287 |
| | RAIAANEADAVTLDAGLVYDAYLAPNNLKPVVAEFYGSKEDPQTFYYAV | |
| | AVVKKCSGFQMNQLRGKKSCHTGLGRSAGWNIPIGLLYCDLPEPRKPLE | |
| | KAVANFFSGSCAPCADGTDFPQLCQLCPGCGCSTLNQYFGYSGAFKCLK | |
| | DGAGDVAFVKHSTIFENLANKADRDQYELLCLDNTRKPVDEYKDCHLAQ | |
| | VPSHTVVARSMGGKEDLIWELLNQAQEHFGKDKSKEFQLFSSPHGKDLL | |
| | FKDSAHGFLKVPPRMDAKMYLGYEYVTAIRNLREGTCPEAPTDECKPVK | |
| | WCALSHHERLKCDEWSVNSVGKIECVSAETTEDCIAKIMNGEADAMSLD | |
| | GGFVYIAGKCGLVPVLAENYNKSDNCEDTPEAGYFAVAVVKKSASDLTW | |
| | DNLKGKKSCHTAVGRTAGWNIPMGLLYNKINHCRFDEFFSEGCAPGSKK | |
| | DSSLCKLCMGSGLNLCEPNNKEGYYGYTGAFRCLVEKGDVAFVKHQTVP | |
| | QNTGGKNPDPWAKNLNEKDYELLCLDGTRKPVEEYANCHLARAPNHAVV | |
| | TRKDKEACVHKILRQQQHLFGSNVTDCSGNFCLFRSETKDLLFRDDTVC | |
| | LAKLHDRNTYEKYLGEEYVKAVGNLRKCSTSSLLEACTFRRP | |
| E | VPDKTVRWCAVSEHEATKCQSFRDHMKSVIPSDGPSVACVKKASYLDCI | 3288 |
| | RAIAANEADAVTLDAGLVYDAYLAPNNLKPVVAEFYGSKEDPQTFYYAV | |
| | AVVKKDSGFQMNQLRGKKSCHTGLGRSAGWNIPIGLLYCDLPEPRKPLE | |
| | KAVANFFSGSCAPCADGCDFPQLCQLCPGCGCSTLNQYFGYSGAFKCLK | |
| | DGAGDVAFVKHSTIFENLANKADRDQYELLCLDNTRKPVDEYKDCHLAQ | |
| | VPSHTVVARSMGGKEDLIWELLNQAQEHFGKDKSKEFQLFSSPHGKDLL | |
| | FKDSAHGFLKVPPRMDAKMYLGYEYVTAIRNLREGTCPEAPTDECKPVK | |
| | WCALSHHERLKCDEWSVNSVGKIECVSAETTEDCIAKIMNGEADAMSLD | |
| | GGFVYIAGKCGLVPVLAENYNKSDNCEDTPEAGYFAVAVVKKSASDLTW | |
| | DNLKGKKSCHTAVGRTAGWNIPMGLLYNKINHCRFDEFFSEGCAPGSKK | |
| | DSSLCKLCMGSGLNLCEPNNKEGYYGYTGAFRCLVEKGDVAFVKHQTVP | |
| | QNTGGKNPDPWAKNLNEKDYELLCLDGTRKPVEEYANCHLARAPNHAVV | |
| | TRKDKEACVHKILRQQQHLFGSNVTDCSGNFCLFRSETKDLLFRDDTVC | |
| | LAKLHDRNTYEKYLGEEYVKAVGNLRKCSTSSLLEACTFRRP | |
| F | VPDKTVRWCAVSEHEATKCQSFRDHMKSVIPSDGPSVACVKKASYLDCI | 3289 |
| | RAIAANEADAVTLDAGLVYDAYLAPNNLKPVVAEFYGSKEDPQTFYYAV | |
| | AVVKKDSGFQMNQLRGKKSCHTGLGRSAGWNIPIGLLYCDLPEPRKPLE | |
| | KAVANFFSGSCAPCADGTDFPQLCQLCPGCGCSTLNQYFGYSGAFKCLK | |
| | DGAGDVAFVKHSTIFENLANKADRDQYELLCLDNTRKPVDEYKDCHLAQ | |
| | VPSHTVVARSMGGKEDLIWELLNQAQEHFGKDKSKEFQLFSSPHGKDLL | |
| | FKDSAHGFLKVPPRMDAKMYLGYEYVTAIRNLREGTCPEAPTDECKPVK | |
| | WCALSHHERLKCDEWSVNSVGKIECVSAETTEDCIAKIMNGEADAMSLD | |
| | GGFVYIAGKCGLVPVLAENYNKADNCEDTPEAGYFAVAVVKKSASDLIW | |
| | DNLKGKKSCHTAVGRTAGWNIPMGLLYNKINHCRFDEFFSEGCAPGSKK | |
| | DSSLCKLCMGSGLNLCEPNNKEGYYGYTGAFRCLVEKGDVAFVKHQTVP | |
| | QNTGGKNPDPWAKNLNEKDYELLCLDGTRKPVEEYANCHLARAPNHAVV | |
| | TRKDKEACVHKILRQQQHLFGSNVTDCSGNFCLFRSETKDLLFRDDTVC | |
| | LAKLHDRNTYEKYLGEEYVKAVGNLRKCSTSSLLEACTFRRP | |
| G | VPDKTVRWCAVSEHEATKCQSFRDHMKSVIPSDGPSVACVKKASYLDCI | 3290 |
| | RAIAANEADAVTLDAGLVYDAYLAPNNLKPVVAEFYGSKEDPQTFYYAV | |
| | AVVKKDSGFQMNQLRGKKSCHTGLGRSAGWNIPIGLLYCDLPEPRKPLE | |
| | KAVANFFSGSCAPCADGTDFPQLCQLCPGCGCSTLNQYFGYSGAFKCLK | |
| | DGAGDVAFVEHSTIFENLANKADRDQYELLCLDNTRKPVDEYKDCHLAQ | |
| | VPSHTVVARSMGGKEDLIWELLNQAQEHFGKDKSKEFQLFSSPHGKDLL | |
| | FKDSAHGFLKVPPRMDAKMYLGYEYVTAIRNLREGTCPEAPTDECKPVK | |
| | WCALSHHERLKCDEWSVNSVGKIECVSAETTEDCIAKIMNGEADAMSLD | |
| | GGFVYIAGKCGLVPVLAENYNKSDNCEDTPEAGYFAVAVVKKSASDLTW | |
| | DNLKGKKSCHTAVGRTAGWNIPMGLLYNKINHCRFDEFFSEGCAPGSKK | |
| | DSSLCKLCMGSGLNLCEPNNKEGYYGYTGAFRCLVEKGDVAFVKHQTVP | |
| | QNTGGKNPDPWAKNLNEKDYELLCLDGTRKPVEEYANCHLARAPNHAVV | |
| | TRKDKEACVHKILRQQQHLFGSNVTDCSGNFCLFRSETKDLLFRDDTVC | |
| | LAKLHDRNTYEKYLGEEYVKAVGNLRKCSTSSLLEACTFRRP | |
| H | VPDKTVRWCAVSEHEATKCQSFRDHMKSVIPSDGPSVACVKKASYLDCI | 3291 |
| | RAIAANEADAVTLDAGLVYDAYLAPNNLKPVVAEFYGSKEDPQTFYYAV | |
| | AVVKKDSGFQMNQLRGKKSCHTGLGRSAGWNIPIGLLYCDLPEPRKPLE | |
| | KAVANFFSGSCAPCADGTDFPQLCQLCPGCGCSTLNQYFGYSGAFKCLK | |
| | DGAGDVAFVEHSTIFENLANKADRDQYELLCLDNTRKPVDEYKDCHLAQ | |
| | VPSHTVVARSMGGKEDLIWELLNQAQEHFGKDKSKEFQLFSSPHGKDLL | |
| | FKDSAHGFLKVPPRMDAKMYLGYEYVTAIRNLREGTCPEAPTDECKPVK | |
| | WCALSHHERLKCDEWSVNSVGKIECVSAETTEDCIAKIMNGEADAMSLD | |
| | GGFVYIAGKCGLVPVLAENYNKSDNCEDTPEAGYFAVAVVKKSASDLTW | |
| | DNLKGKKSCHTAVGRTAGWNIPMGLLYNKINHCRFDEFFSEGCAPGSKK | |
| | DSSLCKLCMGSGLNLCEPNNKEGYYGYTGAFRCLVEKGDVAFVKHQTVP | |
| | QNTGGKNPDPWAKNLNEKDYELLCLDGTRKPVEEYANCHLARAPNHAVV | |
| | TRKDKEACVHKILRQQQHLFGSNVTDCSGNFCLFRSETKDLLFADDTVC | |
| | LAKLHDRNTYEKYLGEEYVKAVGNLRKCSTSSLLEACTFRRP | |
| I | VPDKTVRWCAVSEHEATKCQSFRDHMKSVIPSDGPSVACVKKASYLDCI | 3292 |
| | RAIAANEADAVTLDAGLVYDAYLAPNNLKPVVAEFYGSKEDPQTFYYAV | |
| | AVVKKDSGFQMNQLRGKKSCHTGLGRSAGWNIPIGLLYCDLPEPRKPLE | |
| | KAVANFFSGSCAPCADGTDFPQLCQLCPGCGCSTLNQYFGYSGAFKCLK | |
| | DGAGDVAFVEHSTIFENLANKADRDQYELLCLDNTRKPVDEYKDCHLAQ | |
| | VPSHTVVARSMGGKEDLIWELLNQAQEHFGKDKSKEFQLFSSPHGKDLL | |
| | FKDSAHGFLKVPPRMDAKMYLGYEYVTAIRNLREGTCPEAPTDECKPVK | |
| | WCALSHHERLKCDEWSVNSVGKIECVSAETTEDCIAKIMNGEADAMSLD | |
| | GGFVYIAGKCGLVPVLAENYNKSDNCEDTPEAGYFAVAVVKKSASDLTW | |
| | DNLKGKKSCHTAVGRTAGWNIPMGLLYNKINHCRFDEFFSEGCAPGSKK | |
| | DSSLCKLCMGSGLNLCEPNNKEGYYGYTGAFRCLVEKGDVAFVAHQTVP | |
| | QNTGGKNPDPWAKNLNEKDYELLCLDGTRKPVEEYANCHLARAPNHAVV | |
| | TRKDKEACVHKILRQQQHLFGSNVTDCSGNFCLFRSETKDLLERDDTVC | |
| | LAKLHDRNTYEKYLGEEYVKAVGNLRKCSTSSLLEACTERRP | |

In some embodiments, the TFR targeting moiety comprises a TF variant (e.g., a hTF variant) (or a functional fragment or variant thereof). In some embodiments, TF variant (or the functional fragment or variant thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a TF variant set forth in Table 12, wherein the amino acid sequence of the TF variant comprises at least one amino acid variation compared to the amino acid sequence of a reference TF protein (e.g., SEQ ID NO: 3283). In some embodiments, TF variant (or the functional fragment or variant thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 3200-3208, wherein the amino acid sequence of the TF variant comprises at least one amino acid variation compared to the amino acid sequence of a reference TF protein (e.g., SEQ ID NO: 3283).

### Heterologous Moieties

In some embodiments, the TF (e.g., hTF) is operably connected to a heterologous moiety (e.g., an Fc region (e.g., an Fc region described herein (e.g., described herein). In some embodiments, the heterologous moiety is a half-life extension moiety. Exemplary half-life extension moieties include, but are not limited to, an immunoglobulin (e.g., human Ig (hIg)), a fragment of an Ig (e.g., hIg), an Ig (e.g., hIg) constant region, a fragment of an Ig (e.g., hIg) constant region, an Ig (e.g., hIg) Fc region, human serum albumin (HSA), an HSA binding protein or peptide, and polyethylene glycol (PEG) (and polymers thereof). In some embodiments, the heterologous polypeptide is a half-life extension polypeptide. Exemplary half-life extension polypeptides include, but are not limited to, an Ig, a fragment of an Ig, one or more Ig heavy chain constant region, a fragment of an Ig constant region, an Ig Fc region, a hIg, a fragment of a hIg, one or more hIg heavy chain constant region, a fragment of a hIg constant region, a hIg Fc region, human serum albumin (HSA), and an HSA binding protein or peptide. The immunomodulatory protein or polypeptide described herein fused or conjugated to a half-life extending moiety or a half-life extending moiety can be evaluated for their pharmacokinetic properties utilizing standard in vivo methods known in the art.

In some embodiments, the heterologous moiety is a heterologous polypeptide. In some embodiments, the heterologous polypeptide comprises one or more Ig heavy chain constant region (e.g., a CH2 region, a CH3 region, a hinge region, an Fc region). In some embodiments, the Ig is an IgG. In some embodiments, the IgG is IgG1, IgG2, IgG3, or IgG4.

In some embodiments, the heterologous polypeptide comprises or consists of an IgG CH2 region and an IgG CH3 region. In some embodiments, the heterologous polypeptide comprises or consists of a partial IgG hinge region, IgG CH2 region, and IgG CH3 region. In some embodiments, the heterologous polypeptide comprises or consists of an IgG hinge region, IgG CH2 region, and IgG CH3 region. In some embodiments, the heterologous polypeptide comprises or consists of an IgG1 CH2 region and an IgG1 CH3 region. In some embodiments, the heterologous polypeptide comprises or consists of a partial IgG1 hinge region, IgG1 CH2 region, and IgG1 CH3 region. In some embodiments, the heterologous polypeptide comprises or consists of an IgG1 hinge region, IgG1 CH2 region, and IgG1 CH3 region. In some embodiments, the heterologous polypeptide comprises or consists of an IgG4 CH2 region and an IgG4 CH3 region. In some embodiments, the heterologous polypeptide comprises or consists of a partial IgG4 hinge region, IgG4 CH2 region, and IgG4 CH3 region. In some embodiments, the heterologous polypeptide comprises or consists of an IgG4 hinge region, IgG4 CH2 region, and IgG4 CH3 region.

In some embodiments, the heterologous polypeptide comprises or consists of an Ig Fc region. In some embodiments, the Ig Fc region comprises or consists of at least a portion of a hinge region, a CH2 region, and a CH3 region. In some embodiments, the Ig Fc region comprises or consists of a hinge region, a CH2 region, and a CH3 region. In some embodiments, the Ig Fc region comprises or consists of at least a portion of an IgG hinge region, an IgG CH2 region, and an IgG CH3 region. In some embodiments, the Ig Fc region comprises or consists of an IgG hinge region, an IgG CH2 region, and an IgG CH3 region. In some embodiments, the Ig Fc region comprises or consists of at least a portion of an IgG1 hinge region, an IgG1 CH2 region, and an IgG1 CH3 region. In some embodiments, the Ig Fc region comprises or consists of an IgG1 hinge region, an IgG1 CH2 region, and an IgG1 CH3 region. In some embodiments, the Ig Fc region comprises or consists of at least a portion of an IgG4 hinge region, an IgG4 CH2 region, and an IgG4 CH3 region. In some embodiments, the Ig Fc region comprises or consists of an IgG4 hinge region, an IgG4 CH2 region, and an IgG4 CH3 region.

In some embodiments, the heterologous polypeptide comprises one or more hIg heavy chain constant regions (e.g., a CH2 region, a CH3 region, a hinge region, an Fc region). In some embodiments, the hIg is a human IgG (hIgG). In some embodiments, the hIgG is hIgG1, IgG2, IgG3, or IgG4. In some embodiments, the hIgG is IgG1 or IgG4. In some embodiments, the hIgG is hIgG1. In some embodiments, the hIgG is hIgG4.

In some embodiments, the heterologous polypeptide comprises or consists of a hIgG CH2 region and a hIgG CH3 region. In some embodiments, the heterologous polypeptide comprises or consists of a partial hIgG hinge region, hIgG CH2 region, and hIgG CH3 region. In some embodiments, the heterologous polypeptide comprises or consists of a hIgG hinge region, hIgG CH2 region, and hIgG CH3 region. In some embodiments, the heterologous polypeptide comprises or consists of a hIgG1 CH2 region and a hIgG1 CH3 region. In some embodiments, the heterologous polypeptide comprises or consists of a partial hIgG1 hinge region, hIgG1 CH2 region, and hIgG1 CH3 region. In some embodiments, the heterologous polypeptide comprises or consists of a hIgG1 hinge region, hIgG1 CH2 region, and hIgG1 CH3 region. In some embodiments, the heterologous polypeptide comprises or consists of a hIgG4 CH2 region and a hIgG4 CH3 region. In some embodiments, the heterologous polypeptide comprises or consists of a partial hIgG4 hinge region, hIgG4 CH2 region, and hIgG4 CH3 region. In some embodiments, the heterologous polypeptide comprises or consists of a hIgG4 hinge region, hIgG4 CH2 region, and hIgG4 CH3 region.

In some embodiments, the heterologous polypeptide comprises or consists of a hIg Fc region. In some embodiments, the hIg Fc region comprises or consists of at least a portion of a hinge region, a CH2 region, and a CH3 region. In some embodiments, the hIg Fc region comprises or consists of a hinge region, a CH2 region, and a CH3 region. In some embodiments, the hIg Fc region comprises or consists of at least a portion of a hIgG hinge region, a hIgG CH2 region, and a hIgG CH3 region. In some embodiments, the hIg Fc region comprises or consists of a hIgG hinge region, a hIgG CH2 region, and a hIgG CH3 region. In some embodiments, the hIg Fc region comprises or consists of at least a portion of a hIgG1 hinge region, a hIgG1 CH2 region, and a hIgG1 CH3 region. In some embodiments, the hIg Fc region comprises or consists of a hIgG1 hinge region, a hIgG1 CH2 region, and a hIgG1 CH3 region. In some embodiments, the hIg Fc region comprises or consists of at least a portion of a hIgG4 hinge region, a hIgG4 CH2 region, and a hIgG4 CH3 region. In some embodiments, the hIg Fc region comprises or consists of a hIgG4 hinge region, a hIgG4 CH2 region, and a hIgG4 CH3 region.

### TFR Binding Peptides and Antibody-Like Scaffolds

In some embodiments, the TFR targeting moiety comprises a peptide that specifically binds TFR (e.g., hTFR). TFR (e.g., hTFR (e.g., hTFR1)) binding peptides that can be employed in the conjugates described herein are known in the art. For example, see, e.g., U.S. Pat. Nos. 6,743,893 and 8,399,653, the entire contents of each of which is incorporated herein by reference for all purposes.

In some embodiments, the TFR targeting moiety comprises an antibody-like scaffold. Anti-TFR antibody like scaffolds that can be employed in the conjugates described herein are known in the art. See, e.g., WO2023023031 (describing anti-TFR cysteine dense peptides) and WO2021076546 (describing TFR binding fibronectin type III domains), the entire contents of each of which is incorporated herein by reference for all purposes.

### Exemplary TFR Binding Peptides and Antibody-Like Scaffolds

The amino acid sequence of exemplary TFR specific antibody like scaffolds is provided in Table 13.

TABLE 13
The Amino Acid Sequence of Exemplary TFR Specific Peptides and Antibody
Like Scaffolds.

| | | SEQ |
|---|---|---|
| Description | Amino Acid Sequence | ID NO |
| TFR Binding Peptides (see, e.g., US6743893 and US8399653) | | |
| A | HAIYPRH | 3293 |
| B | THRPPMWSPVWP | 3294 |
| C | GGGDIMGEWGNEIFGAIAGFLGGGRRRRRRRRR | 3295 |
| TFR Binding Cysteine Dense Peptides (see, e.g., WO2023023031) | | |
| A | REGCASRCTKYNAELEKCEARVSSMSNTEETCVQELFDLLHCVDHCVSQ | 3296 |
| B | REGCASRCTKYNAELEKCEARVMSMSNTEEDCEQELEDLLHCLDHCHSQ | 3297 |
| C | REGCASRCTKYNAELEKCEARVVSMSNTEEDCEQELEDLLHCLDHCHSQ | 3298 |
| D | REGCASRCTKYNAELEKCEARVMSMSNTEETCEQELEDLLHCLDHCHSQ | 3299 |
| E | REGCASRCTKYNAELEKCEARVMSMSNTEEDCVQELEDLLHCLDHCHSQ | 3300 |
| F | REGCASRCTKYNAELEKCEARVMSMSNTEEDCEQELFDLLHCLDHCHSQ | 3301 |
| G | REGCASRCTKYNAELEKCEARVMSMSNTEEDCEQELEDLLHCVDHCHSQ | 3302 |
| H | REGCASRCTKYNAELEKCEARVMSMSNTEEDCEQELEDLLHCLDHCVSQ | 3303 |
| I | REGCASRCTKYNAELEKCEARVVSMSNTEETCEQELEDLLHCLDHCHSQ | 3304 |
| J | REGCASRCTKYNAELEKCEARVVSMSNTEEDCVQELEDLLHCLDHCHSQ | 3305 |
| K | REGCASRCTKYNAELEKCEARVVSMSNTEEDCEQELFDLLHCLDHCHSQ | 3306 |
| L | REGCASRCTKYNAELEKCEARVVSMSNTEEDCEQELEDLLHCVDHCHSQ | 3307 |
| M | REGCASRCTKYNAELEKCEARVVSMSNTEEDCEQELEDLLHCLDHCVSQ | 3308 |
| N | REGCASRCTKYNAELEKCEARVMSMSNTEETCVQELEDLLHCLDHCHSQ | 3309 |
| O | REGCASRCTKYNAELEKCEARVMSMSNTEETCEQELFDLLHCLDHCHSQ | 3310 |
| P | REGCASRCTKYNAELEKCEARVMSMSNTEETCEQELEDLLHCVDHCHSQ | 3311 |
| Q | REGCASRCTKYNAELEKCEARVMSMSNTEETCEQELEDLLHCLDHCVSQ | 3312 |
| R | REGCASRCTKYNAELEKCEARVMSMSNTEEDCVQELFDLLHCLDHCHSQ | 3313 |
| S | REGCASRCTKYNAELEKCEARVMSMSNTEEDCVQELEDLLHCVDHCHSQ | 3314 |
| T | REGCASRCTKYNAELEKCEARVMSMSNTEEDCVQELEDLLHCLDHCVSQ | 3315 |
| U | REGCASRCTKYNAELEKCEARVMSMSNTEEDCEQELFDLLHCVDHCHSQ | 3316 |
| V | REGCASRCTKYNAELEKCEARVMSMSNTEEDCEQELFDLLHCLDHCVSQ | 3317 |
| W | REGCASRCTKYNAELEKCEARVMSMSNTEEDCEQELEDLLHCVDHCVSQ | 3318 |
| X | REGCASRCMKYNAELEKCEARVMSMSNTEEDCEQELEDLLHCLDHCHSQ | 3319 |
| Y | REGCASRCTKYNDELEKCEARVMSMSNTEEDCEQELEDLLHCLDHCHSQ | 3320 |
| Z | REGCASRCTKYNAELEKCEARMMSMSNTEEDCEQELEDLLHCLDHCHSQ | 3321 |

TFR binding fibronectin type IIIdomains (see, e.g., WO2021076546)

| | | |
|---|---|---|
| A | MLPAPKNLVVSRVTEDSARLSWTAPDAAFDSFVIEYREGAWYGEAIV | 3322 |
| | LTVPGSERSYDLIGLKPGTEYAVYIPGVKGGPRSFPLSAIFTT | |
| B | MLPAPKNLVVSRVTEDSARLSWTAPDAAFDSFAIAYVEWWKLGEAIV | 3323 |
| | LTVPGSERSYDLTGLKPGTEYVVPIPGVKGGGHSSPLSAIFTT | |
| C | MLPAPKNLVVSRVTEDSARLSWTAPDAAFDSFYIYYYESSGTGEAIVLT | 3324 |
| | VPGSERSYDLTGLKPGTEYFVDIGGVKGGSYSLPLSAIFTT | |
| D | MLPAPKNLVVSRVTEDSARLSWTAPDAAFDSFPIYYWEVFPAGEAIEL | 3325 |
| | DVPGSERSYDLTGLKPGTEYFVRIEGVKGGASSYPLRAEFTT | |
| E | MLPAPKNLVVSRVTEDSARLSWTAPDAAFDSFFIWYWEKSVDGEAIV | 3326 |
| | LTVPGSERSYDLTGLKPGTEYNVGIQGVKGGTPSDPLSAIFTT | |
| F | MLPAPKNLVVSRVTEDSARLSWTAPDAAFDSFIIWYAEWVNDGEAIV | 3327 |
| | LTVPGSERSYDLIGLKPGTEYRVEITGVKGGTWSRPLSAIFTT | |
| G | MLPAPKNLVVSRVTEDSARLSWTAPDAAFDSFAIEYYEPVPAGEAIYL | 3328 |
| | DVPGSERSYDLTGLKPGTEYDVTIYGVKGGYYSHPLFASFTT | |
| H | MLPAPKNLVVSRVTEDSARLSWTAPDAAFDSFWIEYFEWTVGGEAIV | 3329 |
| | LTVPGSERSYDLTGLKPGTEYYVSIYGVKGGWLSPPLSAIFTT | |
| I | MLPAPKNLVVSRVTEDSARLSWTAPDAAFDSFHISYEETPVVGEAIYL | 3330 |
| | RVPGSERSYDLTGLKPGTEYTVAIHGVKGGRESTPLIAPFTT | |
| J | MLPAPKNLVVSRVTEDSARLSWTAPDAAFDSFTIHYWEFDPPGEAIV | 3331 |
| | LTVPGSERSYDLTGLKPGTEYTVYIEGVKGGWWSKPLSAIFTT | |
| K | MLPAPKNLVVSRVTEDSARLSWTAPDAAFDSFVIEYWERTQPGEAIV | 3332 |
| | LTVPGSERSYDLIGLKPGTEYDVWISGVKGGKWSEPLSAIFTT | |
| L | MLPAPKNLVVSRVTEDSARLSWTAPDAAFDSFLIRYWEWYVLGEAIV | 3333 |
| | LTVPGSERSYDLTGLKPGTEYYVEISGVKGGWQSWPLSAIFTT | |
| M | MLPAPKNLVVSRVTEDSARLSWTAPDAAFDSFYIGYLEPGDNGEAIVL | 3334 |
| | TVPGSERSYDLTGLKPGTEYNVSIGGVKGGLGSYPLSAIFTT | |
| N | MLPAPKNLVVSRITEDSARLSWTAPDAAFDSFGIYYYEWWSTGEAIVL | 3335 |
| | TVPGSERSYDLTGPKPGTEYYVKISGVKGGYRSYPLSAIFTT | |
| O | MLPAPKNLVVSRVTEDSARLSWTAPDAAFDSFRISYYEWYDLGEAIVL | 3336 |
| | TVPGSERSYDLTGLKPGTEYWVDIAGVKGGYYSYPLSAIFTT | |
| P | MLPAPKNLVVSRVTEDSARLSWTAPDAAFDSFFIGYLEPQPPGEAIVL | 3337 |
| | TVPGSERSYDLTGLKPGTEYNVTIQGVKGGFPSMPLSAIFTT | |
| Q | MLPAPKNLVVSRVTEDSARLSWTAPDAAFDSFFISYFEGWASGEAIHL | 3338 |
| | YVPGSERSYDLTGLKPGTEYSVHIQGVKGGQPSTPLSAIFTT | |
| R | MLPAPKNLVVSRITEDSARLSWTAPDAAFDSFDIPYGEFDTIGEAIVLT | 3339 |
| | VPGSERSYDLTGLKPGTEYDVYIEGVKGGHLSWPLSAIFTT | |
| S | MLPAPKNLVVSRVTEDSARLSWTAPDAAFDSFGIQYNEFVERGEAIVL | 3340 |
| | TVPGSERSYDLIGLKPGTEYFVPISGVKGGDDSRPLSAIFTT | |
| T | MLPAPKNLVVSRVTEDSARLSWTAPDAAFDSFWIEYWEVVGFGEAI | 3341 |
| | VLTVPGSERSYDLTGLKPGTEYWVGIYGVKGGNPSVPLSAIFTT | |

In some embodiments, the TFR targeting moiety comprises a TFR specific peptide or an antibody like scaffold (or a functional fragment or variant thereof). In some embodiments, the TFR targeting moiety comprises a TFR specific peptide (or a functional fragment or variant thereof). In some embodiments, the TFR specific peptide (or the functional fragment or variant thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a TFR specific peptide (or the functional fragment or variant thereof) set forth in Table 13. In some embodiments, the TFR specific peptide (or the functional fragment or variant thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 3293-3341. In some embodiments, the TFR targeting moiety comprises an antibody like scaffold (or a functional fragment or variant thereof). In some embodiments, the antibody like scaffold (or the functional fragment or variant thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of antibody like scaffold (or the functional fragment or variant thereof) set forth in Table 13. In some embodiments, the antibody like scaffold (or the functional fragment or variant thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 3293-3341.

### Heterologous Moieties

In some embodiments, the TFR binding peptide or antibody-like scaffold is operably connected to a heterologous moiety (e.g., an Fc region (e.g., an Fc region described herein (e.g., described herein)). In some embodiments, the heterologous moiety is a half-life extension moiety. Exemplary half-life extension moieties include, but are not limited to, an immunoglobulin (e.g., human Ig (hIg)), a fragment of an Ig (e.g., hIg), an Ig (e.g., hIg) constant region, a fragment of an Ig (e.g., hIg) constant region, an Ig (e.g., hIg) Fc region human transferrin, human serum albumin (HSA), an HSA binding protein or peptide, and polyethylene glycol (PEG) (and polymers thereof). In some embodiments, the heterologous polypeptide is a half-life extension polypeptide. Exemplary half-life extension polypeptides include, but are not limited to, an Ig, a fragment of an Ig, one or more Ig heavy chain constant region, a fragment of an Ig constant region, an Ig Fc region, a hIg, a fragment of a hIg, one or more hIg heavy chain constant region, a fragment of a hIg constant region, a hIg Fc region, human serum albumin (HSA), and an HSA binding protein or peptide. The immunomodulatory protein or polypeptide described herein fused or conjugated to a half-life extending moiety or a half-life extending moiety can be evaluated for their pharmacokinetic properties utilizing standard in vivo methods known in the art.

In some embodiments, the heterologous moiety is a heterologous polypeptide. In some embodiments, the heterologous polypeptide comprises one or more Ig heavy chain constant region (e.g., a CH2 region, a CH3 region, a hinge region, an Fc region). In some embodiments, the Ig is an IgG. In some embodiments, the IgG is IgG1, IgG2, IgG3, or IgG4.

In some embodiments, the heterologous polypeptide comprises or consists of an IgG CH2 region and an IgG CH3 region. In some embodiments, the heterologous polypeptide comprises or consists of a partial IgG hinge region, IgG CH2 region, and IgG CH3 region. In some embodiments, the heterologous polypeptide comprises or consists of an IgG hinge region, IgG CH2 region, and IgG CH3 region. In some embodiments, the heterologous polypeptide comprises or consists of an IgG1 CH2 region and an IgG1 CH3 region. In some embodiments, the heterologous polypeptide comprises or consists of a partial IgG1 hinge region, IgG1 CH2 region, and IgG1 CH3 region. In some embodiments, the heterologous polypeptide comprises or consists of an IgG1 hinge region, IgG1 CH2 region, and IgG1 CH3 region. In some embodiments, the heterologous polypeptide comprises or consists of an IgG4 CH2 region and an IgG4 CH3 region. In some embodiments, the heterologous polypeptide comprises or consists of a partial IgG4 hinge region, IgG4 CH2 region, and IgG4 CH3 region. In some embodiments, the heterologous polypeptide comprises or consists of an IgG4 hinge region, IgG4 CH2 region, and IgG4 CH3 region.

In some embodiments, the heterologous polypeptide comprises or consists of an Ig Fc region. In some embodiments, the Ig Fc region comprises or consists of at least a portion of a hinge region, a CH2 region, and a CH3 region. In some embodiments, the Ig Fc region comprises or consists of a hinge region, a CH2 region, and a CH3 region. In some embodiments, the Ig Fc region comprises or consists of at least a portion of an IgG hinge region, an IgG CH2 region, and an IgG CH3 region. In some embodiments, the Ig Fc region comprises or consists of an IgG hinge region, an IgG CH2 region, and an IgG CH3 region. In some embodiments, the Ig Fc region comprises or consists of at least a portion of an IgG1 hinge region, an IgG1 CH2 region, and an IgG1 CH3 region. In some embodiments, the Ig Fc region comprises or consists of an IgG1 hinge region, an IgG1 CH2 region, and an IgG1 CH3 region. In some embodiments, the Ig Fc region comprises or consists of at least a portion of an IgG4 hinge region, an IgG4 CH2 region, and an IgG4 CH3 region. In some embodiments, the Ig Fc region comprises or consists of an IgG4 hinge region, an IgG4 CH2 region, and an IgG4 CH3 region.

In some embodiments, the heterologous polypeptide comprises one or more hIg heavy chain constant regions (e.g., a CH2 region, a CH3 region, a hinge region, an Fc region). In some embodiments, the hIg is a human IgG (hIgG). In some embodiments, the hIgG is hIgG1, IgG2, IgG3, or IgG4. In some embodiments, the hIgG is IgG1 or IgG4. In some embodiments, the hIgG is hIgG1. In some embodiments, the hIgG is hIgG4.

In some embodiments, the heterologous polypeptide comprises or consists of a hIgG CH2 region and a hIgG CH3 region. In some embodiments, the heterologous polypeptide comprises or consists of a partial hIgG hinge region, hIgG CH2 region, and hIgG CH3 region. In some embodiments, the heterologous polypeptide comprises or consists of a hIgG hinge region, hIgG CH2 region, and hIgG CH3 region. In some embodiments, the heterologous polypeptide comprises or consists of a hIgG1 CH2 region and a hIgG1 CH3 region. In some embodiments, the heterologous polypeptide comprises or consists of a partial hIgG1 hinge region, hIgG1 CH2 region, and hIgG1 CH3 region. In some embodiments, the heterologous polypeptide comprises or consists of a hIgG1 hinge region, hIgG1 CH2 region, and hIgG1 CH3 region. In some embodiments, the heterologous polypeptide comprises or consists of a hIgG4 CH2 region and a hIgG4 CH3 region. In some embodiments, the heterologous polypeptide comprises or consists of a partial hIgG4 hinge region, hIgG4 CH2 region, and hIgG4 CH3 region. In some embodiments, the heterologous polypeptide comprises or consists of a hIgG4 hinge region, hIgG4 CH2 region, and hIgG4 CH3 region.

In some embodiments, the heterologous polypeptide comprises or consists of a hIg Fc region. In some embodiments, the hIg Fc region comprises or consists of at least a portion of a hinge region, a CH2 region, and a CH3 region. In some embodiments, the hIg Fc region comprises or consists of a hinge region, a CH2 region, and a CH3 region. In some embodiments, the hIg Fc region comprises or consists of at least a portion of a hIgG hinge region, a hIgG CH2 region, and a hIgG CH3 region. In some embodiments, the hIg Fc region comprises or consists of a hIgG hinge region, a hIgG CH2 region, and a hIgG CH3 region. In some embodiments, the hIg Fc region comprises or consists of at least a portion of a hIgG1 hinge region, a hIgG1 CH2 region, and a hIgG1 CH3 region. In some embodiments, the hIg Fc region comprises or consists of a hIgG1 hinge region, a hIgG1 CH2 region, and a hIgG1 CH3 region. In some embodiments, the hIg Fc region comprises or consists of at least a portion of a hIgG4 hinge region, a hIgG4 CH2 region, and a hIgG4 CH3 region. In some embodiments, the hIg Fc region comprises or consists of a hIgG4 hinge region, a hIgG4 CH2 region, and a hIgG4 CH3 region.

### Anti-TFR (e.g., Anti-TFR1) Antibodies

In some embodiments, the TFR targeting moiety is an anti-TFR antibody (e.g., an anti-hTFR antibody) (e.g., an anti-hTFR1 antibody)). In some embodiments, the antibody comprises or consists of a full-length antibody, Fab, Fab′, F(ab′)2, Fab-Fc, scFv, scFv-Fc, (scFv)₂-Fc, Fv, a single domain antibody (sdAb) (e.g., a VHH), a sdAb-Fc (e.g., a VHH-Fc), (sdAb)₂ (e.g., a (VHH)₂, or a (sdAb)₂-Fc (e.g., (VHH)₂-Fc). In some embodiments, the antibody comprises or consists of a full-length antibody, Fab, Fab′, F(ab′)2, Fab-Fc, scFv, scFv-Fc, (scFv)₂-Fc, sdAb-Fc (e.g., a VHH-Fc), or (sdAb)₂-Fc (e.g., (VHH)₂-Fc). In some embodiments, the antibody comprises or consists of a full-length antibody. In some embodiments, the antibody comprises or consists of a Fab. In some embodiments, the antibody comprises or consists of a F(ab′)2. In some embodiments, the antibody comprises or consists of a Fab-Fc. In some embodiments, the antibody comprises or consists of a scFv-Fc. In some embodiments, the antibody comprises or consists of a (scFv)₂-Fc. In some embodiments, the antibody comprises or consists of a sdAb-Fc (e.g., a VHH-Fc). In some embodiments, the antibody comprises or consists of a o(sdAb)₂-Fc (e.g., (VHH)₂-Fc)

In some embodiments, the antibody is an IgG1, IgG2, IgG3, or IgG4 antibody. In some embodiments, the antibody is an IgG1 or IgG4 antibody. In some embodiments, the antibody is an IgG1 antibody. In some embodiments, the antibody is an IgG4 antibody. In some embodiments, the antibody is a hIgG1, hIgG2, hIgG3, or hIgG4 antibody. In some embodiments, the antibody is a hIgG1 or hIgG4 antibody. In some embodiments, the antibody is a hIgG1 antibody. In some embodiments, the antibody is a hIgG4 antibody.

### Exemplary Anti-TFR (e.g., Anti-TFR1) Antibodies

In some embodiments, the targeting moiety (e.g., the hematopoietic cell (e.g., erythroid precursor cell)-targeting moiety) comprises an anti-TFR antibody. Anti-hTFR1 antibodies that can be employed in the conjugates described herein are known in the art.

Exemplary anti-TFR1 antibodies known in the art that can be employed in the conjugates described herein include, but are not limited to, e.g., OKT9 (see, e.g., U.S. Pat. No. 4,364,934); M11, M23, M27, B84 (see, e.g., WO2015098989 and U.S. Pat. No. 9,994,641); 7A4, 8A2, 15D2, 10D11, 7B10, 15G11, 16G5, 13C3, 16G4, 16F6, 7G7, 4C2, 1B12, and 13D4 (see, e.g., WO2016081643 and U.S. Pat. No. 9,708,406); 8D3 (see, e.g., US2010/077498 and Lee et al. “Targeting Rat Anti-8D3 Mouse Transferrin Receptor Monoclonal Antibodies through Blood-Brain Barrier in Mouse” 2000, J Pharmacol. Exp. Ther., 292: 1048-1052); OX26 (see, e.g., Haobam, B. et al. 2014. Rab17-mediated recycling endosomes contribute to autophagosome formation in response to Group A Streptococcus invasion. Cellular microbiology. 16: 1806-21); DF1513 (see, e.g., Ortiz-Zapater E et al. Trafficking of the human transferrin receptor in plant cells: effects of tyrphostin A23 and brefeldin A. Plant J 48:757-70 (2006)); the following commercially available clones (e.g., Novus Biologicals) 1A1B2,661G1, MEM-189, JF0956, 29806, 1A1B2, TFRC/1818, 1E6, 66Ig10, TFRC/1059, Q1/71, 23D10, 13E4, TFRC/1149, ER-MP21, YTA74.4, BU54, 2B6, RI7 217; BA120g (see, e.g., US20110311544A1 and U.S. Pat. No. 7,572,895); B3/25 and T58/30 (see, e.g., Trowbridge, I. S. et al. “Anti-transferrin receptor monoclonal antibody and toxin-antibody conjugates affect growth of human tumour cells.” Nature, 1981, volume 294, pages 171-173); the following commercially available clones (e.g., BioXcell) R17 217.1.3, 5E9C11, OKT9 (BE0023 clone), BK19.9, B3/25, T56/14 and T58/1 (see, e.g., Gatter, K. C. et al. “Transferrin receptors in human tissues: their distribution and possible clinical relevance.” J Clin Pathol. 1983 May; 36(5):539-45); 5E9C11; R17 217.1.3 (available from BioXcell), BE0175 (available from BioXcell); the entire contents of each of which is incorporated herein by reference for all purposes.

Exemplary anti-TFR (e.g., hTFR (e.g., hTFR1)) antibodies that can be employed in the conjugates described herein are described in e.g., WO2023283531; WO2021154477A1; WO2020132584A1; WO2021154476A1; WO2021150382A1; WO2023023031A2; WO2021146256A1; WO2021142275A1; US20220017635A1; WO2016207240A1; U.S. Pat. No. 11,267,896B2; US20220143206A1, U.S. Pat. No. 11,028,179B2; U.S. Pat. No. 11,286,305B2; WO2023087017A1; WO2023086864A1; WO2023044398A1; WO2023039611A2; WO2023034409A1; WO2023283623A1; WO2023283624A2; WO2023283619A2; WO2023283620A1; WO2023283615A1; WO2023283613A1; WO2023283614A2; U.S. Pat. No. 11,672,872B2; U.S. Pat. No. 11,648,318B2; WO2022271549A1; WO2022201122A1; WO2022174114A1; WO2022026152A2; WO2022020107A1; WO2022020106A1; WO2022020105A1; WO2022020108A; WO2022020109A1; WO2021205358A1; US20230174646A1; US20210299266A1; WO2021195469A1; U.S. Pat. No. 11,446,387B2; US20220409735A1; US20210301290A1; US20210369762A1; U.S. Pat. No. 11,525,137B2; U.S. Pat. No. 11,555,190B2; U.S. Pat. No. 11,111,308B2; U.S. Pat. No. 10,550,188B2; U.S. Pat. No. 10,508,151B2; US20160208008A1; US20150291697A1; US20130171061A1; U.S. Pat. No. 9,562,230B2; U.S. Pat. No. 7,976,841B2; U.S. Pat. No. 4,364,934; WO2015098989; U.S. Pat. No. 9,994,641; WO2016081643; U.S. Pat. No. 9,708,406; US2010077498; US20110311544; U.S. Pat. No. 7,572,895; WO2019075417; US20060286030A1; US20190240346A1; US20130216476A1; WO2023283531; US20130177579A1; U.S. Pat. No. 9,598,496B2; US20130045206A1; US20060039908A1; U.S. Pat. Nos. 6,015,555A; 6,008,326A; 5,648,469A; EP79696B1; WO2023034409A1; U.S. Pat. Nos. 4,364,934; 8,409,573; 9,708,406; 9,611,323; WO2015098989; Schneider C. et al. “Structural features of the cell surface receptor for transferrin that is recognized by the monoclonal antibody OKT9.” J Biol Chem. 1982, 257:14, 8516-8522; Lee et al. “Targeting Rat Anti-Mouse Transferrin Receptor Monoclonal Antibodies through Blood-Brain Barrier in Mouse” 2000, J Pharmacol. Exp. Ther., 292: 1048-1052; Lee et al. “Targeting Rat Anti-8D3 Mouse Transferrin Receptor Monoclonal Antibodies through Blood-Brain Barrier in Mouse” 2000, J Pharmacol. Exp. Ther., 292: 1048-1052; Haobam, B. et al. 2014. Rab17-mediated recycling endosomes contribute to autophagosome formation in response to Group A Streptococcus invasion. Cellular microbiology. 16: 1806-21; Ortiz-Zapater E et al. Trafficking of the human transferrin receptor in plant cells: effects of tyrphostin A23 and brefeldin A. Plant J 48:757-70 (2006); Trowbridge, I. S. et al. “Anti-transferrin receptor monoclonal antibody and toxin-antibody conjugates affect growth of human tumour cells.” Nature, 1981, volume 294, pages 171-173; Gatter, K. C. et al. “Transferrin receptors in human tissues: their distribution and possible clinical relevance.” J Clin Pathol. 1983 May; 36(5):539-4; the entire contents of each of which are incorporated herein by reference for all purposes.

The amino acid sequence of exemplary anti-hTFR antibodies that can be utilized in the conjugates described herein is provided in Table 14. The CDRs of the anti-hTFR antibodies in Table 14, are denoted according to Kabat. A person of ordinary skill in the art would be able to determine the CDRs as defined by another scheme, e.g., Chothia, IMGT, using ordinary methods known in the art.

TABLE 14
The Amino Acid Sequence of Exemplary Anti-hTFR Antibodies.

| | | SEQ ID | |
|---|---|---|---|
| Description | | NO | Amino Acid Sequence |
| Full-Length anti- | VH CDR1 | 3342 | NYWLG |
| TFR1 mAb2 | VH CDR2 | 3343 | DIYPGGDYPTYSEKFKV |
| | VH CDR3 | 3344 | SGNYDEVAY |
| | VL CDR1 | 3345 | RSSQSLVHSNGNTYLH |
| | VL CDR2 | 3346 | KVSNRFS |
| | VL CDR3 | 3347 | SQSTHVPWT |
| | VH | 3348 | EVQLVQSGAEVKKPGESLKISCKGSGYSFMNYWLGWVRQMP |
| | | | GKGLEWMGDIYPGGDYPTYSEKFKVQVTISADKSISTAYLQ |
| | | | LSSLKASDTAMYYCARSGNYDEVAYWGQGTLVTVSS |
| | VL | 3349 | DIVMTQTPLSLSVTPGQPASISCRSSQSLVHSNGNTYLHWY |
| | | | LQKPGQSPQLLIYKVSNRFSGVPDRFSGSGSGTDFTLKISR |
| | | | VEAEDVGVYYCSQSTHVPWTFGQGTKVEIK |
| | HC Immature | 3350 | MGWSCIILFLVATATGVHSEVQLVQSGAEVKKPGESLKISC |
| | Sigal | | KGSGYSFMNYWLGWVRQMPGKGLEWMGDIYPGGDYPTYSEK |
| | Sequence | | FKVQVTISADKSISTAYLQLSSLKASDTAMYYCARSGNYDE |
| | Underlined | | VAYWGQGTLVTVSSASTKGPSVFPLAPCSRSTSESTAALGC |
| | | | LVKDYFPEPVTVSWNSGALTSGVHTFPAVLQSSGLYSLSSV |
| | | | VTVPSSSLGTKTYTCNVDHKPSNTKVDKRVESKYGPPCPPC |
| | | | PAPEFEGGPSVFLFPPKPKDTLMISRTPEVTCVVVDVSQED |
| | | | PEVQFNWYVDGVEVHNAKTKPREEQFNSTYRVVSVLTVLHQ |
| | | | DWLNGKEYKCKVSNKGLPSSIEKTISKAKGQPREPQVYTLP |
| | | | PSQEEMTKNQVSLTCLVKGFYPSDIAVEWESNGQPENNYKT |
| | | | TPPVLDSDGSFFLYSRLTVDKSRWQEGNVFSCSVMHEALHN |
| | | | HYTQKSLSLSLG |
| | LC Immature | 3351 | MGWSCIILFLVATATGVHSDIVMTQTPLSLSVTPGQPASIS |
| | Sigal | | CRSSQSLVHSNGNTYLHWYLQKPGQSPQLLIYKVSNRFSGV |
| | Sequence | | PDRFSGSGSGTDFTLKISRVEAEDVGVYYCSQSTHVPWTFG |
| | Underlined | | QGTKVEIKRTVAAPSVFIFPPSDEQLKSGTASVVCLLNNFY |
| | | | PREAKVQWKVDNALQSGNSQESVTEQDSKDSTYSLSSTLTL |
| | | | SKADYEKHKVYACEVTHQGLSSPVTKSFNRGEC |
| | HC Mature | 3352 | EVQLVQSGAEVKKPGESLKISCKGSGYSFMNYWLGWVRQMP |
| | | | GKGLEWMGDIYPGGDYPTYSEKFKVQVTISADKSISTAYLQ |
| | | | LSSLKASDTAMYYCARSGNYDEVAYWGQGTLVTVSSASTKG |
| | | | PSVFPLAPCSRSTSESTAALGCLVKDYFPEPVTVSWNSGAL |
| | | | TSGVHTFPAVLQSSGLYSLSSVVTVPSSSLGTKTYTCNVDH |
| | | | KPSNTKVDKRVESKYGPPCPPCPAPEFEGGPSVFLFPPKPK |
| | | | DTLMISRTPEVTCVVVDVSQEDPEVQFNWYVDGVEVHNAKT |
| | | | KPREEQFNSTYRVVSVLTVLHQDWLNGKEYKCKVSNKGLPS |
| | | | SIEKTISKAKGQPREPQVYTLPPSQEEMTKNQVSLTCLVKG |
| | | | FYPSDIAVEWESNGQPENNYKTTPPVLDSDGSFFLYSRLTV |
| | | | DKSRWQEGNVFSCSVMHEALHNHYTQKSLSLSLG |
| | LC Mature | 3353 | DIVMTQTPLSLSVTPGQPASISCRSSQSLVHSNGNTYLHWY |
| | | | LQKPGQSPQLLIYKVSNRFSGVPDRFSGSGSGTDFTLKISR |
| | | | VEAEDVGVYYCSQSTHVPWTFGQGTKVEIKRTVAAPSVFIF |
| | | | PPSDEQLKSGTASVVCLLNNFYPREAKVQWKVDNALQSGNS |
| | | | QESVTEQDSKDSTYSLSSTLTLSKADYEKHKVYACEVTHQG |
| | | | LSSPVTKSFNRGEC |
| Full-Length anti- | VH CDR1 | 3354 | SYWMH |
| TFR1 mAb3 | VH CDR2 | 3355 | EINPINGRTNYIEKFKS |
| | VH CDR3 | 3356 | GTRAYHY |
| | VL CDR1 | 3357 | RASDNLYSNLA |
| | VL CDR2 | 3358 | DATNLAD |
| | VL CDR3 | 3359 | QHFWGTPLT |
| | VH | 3360 | EVQLVQSGAEVKKPGASVKVSCKASGYTFTSYWMHWVRQAP |
| | | | GQRLEWIGEINPTNGRTNYIEKFKSRATLTVDKSASTAYME |
| | | | LSSLRSEDTAVYYCARGTRAYHYWGQGTMVTVSS |
| | VL | 3361 | DIQMTQSPSSLSASVGDRVTITCRASDNLYSNLAWYQQKPG |
| | | | KSPKLLVYDATNLADGVPSRFSGSGSGTDYSLKINSLQSED |
| | | | FGTYYCQHFWGTPLTFGAGTKLELKRTV |
| | HC Immature | 3362 | MGWSCIILFLVATATGVHSEVQLVQSGAEVKKPGASVKVSC |
| | Sigal | | KASGYTFTSYWMHWVRQAPGQRLEWIGEINPTNGRTNYIEK |
| | Sequence | | FKSRATLTVDKSASTAYMELSSLRSEDTAVYYCARGTRAYH |
| | Underlined | | YWGQGTMVTVSSASTKGPSVFPLAPCSRSTSESTAALGCLV |
| | | | KDYFPEPVTVSWNSGALTSGVHTFPAVLQSSGLYSLSSVVT |
| | | | VPSSSLGTKTYTCNVDHKPSNTKVDKRVESKYGPPCPPCPA |
| | | | PEFEGGPSVFLFPPKPKDTLMISRTPEVTCVVVDVSQEDPE |
| | | | VQFNWYVDGVEVHNAKTKPREEQFNSTYRVVSVLTVLHQDW |
| | | | LNGKEYKCKVSNKGLPSSIEKTISKAKGQPREPQVYTLPPS |
| | | | QEEMTKNQVSLTCLVKGFYPSDIAVEWESNGQPENNYKTTP |
| | | | PVLDSDGSFFLYSRLTVDKSRWQEGNVFSCSVMHEALHNHY |
| | | | TQKSLSLSLG |
| | LC Immature | 3363 | MGWSCIILFLVATATGVHSDIQMTQSPSSLSASVGDRVTIT |
| | Sigal | | CRASDNLYSNLAWYQQKPGKSPKLLVYDATNLADGVPSRFS |
| | Sequence | | GSGSGTDYSLKINSLQSEDFGTYYCQHFWGTPLTFGAGTKL |
| | Underlined | | ELKRTVAAPSVFIFPPSDEQLKSGTASVVCLLNNFYPREAK |
| | | | VQWKVDNALQSGNSQESVTEQDSKDSTYSLSSTLTLSKADY |
| | | | EKHKVYACEVTHQGLSSPVTKSFNRGEC |
| | HC Mature | 3364 | EVQLVQSGAEVKKPGASVKVSCKASGYTFTSYWMHWVRQAP |
| | | | GQRLEWIGEINPTNGRTNYIEKFKSRATLTVDKSASTAYME |
| | | | LSSLRSEDTAVYYCARGTRAYHYWGQGTMVTVSSASTKGPS |
| | | | VFPLAPCSRSTSESTAALGCLVKDYFPEPVTVSWNSGALTS |
| | | | GVHTFPAVLQSSGLYSLSSVVTVPSSSLGTKTYTCNVDHKP |
| | | | SNTKVDKRVESKYGPPCPPCPAPEFEGGPSVFLFPPKPKDT |
| | | | LMISRTPEVTCVVVDVSQEDPEVQFNWYVDGVEVHNAKTKP |
| | | | REEQFNSTYRVVSVLIVLHQDWLNGKEYKCKVSNKGLPSSI |
| | | | EKTISKAKGQPREPQVYTLPPSQEEMTKNQVSLTCLVKGFY |
| | | | PSDIAVEWESNGQPENNYKTTPPVLDSDGSFFLYSRLTVDK |
| | | | SRWQEGNVFSCSVMHEALHNHYTQKSLSLSLG |
| | LC Mature | 3365 | DIQMTQSPSSLSASVGDRVTITCRASDNLYSNLAWYQQKPG |
| | | | KSPKLLVYDATNLADGVPSRFSGSGSGTDYSLKINSLQSED |
| | | | FGTYYCQHFWGTPLTFGAGTKLELKRTVAAPSVFIFPPSDE |
| | | | QLKSGTASVVCLLNNFYPREAKVQWKVDNALQSGNSQESVT |
| | | | EQDSKDSTYSLSSTLTLSKADYEKHKVYACEVTHQGLSSPV |
| | | | TKSFNRGEC |
| Anti-TFR1 Fab | VH CDR1 | 3354 | SYWMH |
| | VH CDR2 | 3355 | EINPTNGRTNYIEKFKS |
| | VH CDR3 | 3356 | GTRAYHY |
| | VL CDR1 | 3357 | RASDNLYSNLA |
| | VL CDR2 | 3358 | DATNLAD |
| | VL CDR3 | 3359 | QHFWGTPLT |
| | | 3360 | EVQLVQSGAEVKKPGASVKVSCKASGYTFTSYWMHWVRQAP |
| | | | GQRLEWIGEINPINGRTNYIEKFKSRATLTVDKSASTAYME |
| | VH | | LSSLRSEDTAVYYCARGTRAYHYWGQGTMVTVSS |
| | VL | 3361 | DIQMTQSPSSLSASVGDRVTITCRASDNLYSNLAWYQQKPG |
| | | | KSPKLLVYDATNLADGVPSRFSGSGSGTDYSLKINSLQSED |
| | | | FGTYYCQHFWGTPLTFGAGTKLELKRTV |
| | HC Immature | 3362 | MGWSCIILFLVATATGVHSEVQLVQSGAEVKKPGASVKVSC |
| | Sigal | | KASGYTFTSYWMHWVRQAPGQRLEWIGEINPINGRTNYIEK |
| | Sequence | | FKSRATLTVDKSASTAYMELSSLRSEDTAVYYCARGTRAYH |
| | Underlined | | YWGQGTMVTVSSASTKGPSVFPLAPCSRSTSESTAALGCLV |
| | | | KDYFPEPVTVSWNSGALTSGVHTFPAVLQSSGLYSLSSVVT |
| | | | VPSSSLGTKTYTCNVDHKPSNTKVDKRV |
| | LC Immature | 3363 | MGWSCIILFLVATATGVHSDIQMTQSPSSLSASVGDRVTIT |
| | Sigal | | CRASDNLYSNLAWYQQKPGKSPKLLVYDATNLADGVPSRFS |
| | Sequence | | GSGSGTDYSLKINSLQSEDFGTYYCQHFWGTPLTFGAGTKL |
| | Underlined | | ELKRTVAAPSVFIFPPSDEQLKSGTASVVCLLNNFYPREAK |
| | | | VQWKVDNALQSGNSQESVTEQDSKDSTYSLSSTLTLSKADY |
| | | | EKHKVYACEVTHQGLSSPVTKSFNRGEC |
| | HC Mature | 3366 | EVQLVQSGAEVKKPGASVKVSCKASGYTFTSYWMHWVRQAP |
| | | | GQRLEWIGEINPTNGRTNYIEKFKSRATLTVDKSASTAYME |
| | | | LSSLRSEDTAVYYCARGTRAYHYWGQGTMVTVSSASTKGPS |
| | | | VFPLAPCSRSTSESTAALGCLVKDYFPEPVTVSWNSGALTS |
| | | | GVHTFPAVLQSSGLYSLSSVVTVPSSSLGTKTYTCNVDHKP |
| | | | SNTKVDKRV |
| | LC Mature | 3365 | DIQMTQSPSSLSASVGDRVTITCRASDNLYSNLAWYQQKPG |
| | | | KSPKLLVYDATNLADGVPSRFSGSGSGTDYSLKINSLQSED |
| | | | FGTYYCQHFWGTPLTFGAGTKLELKRTVAAPSVFIFPPSDE |
| | | | QLKSGTASVVCLLNNFYPREAKVQWKVDNALQSGNSQESVT |
| | | | EQDSKDSTYSLSSTLTLSKADYEKHKVYACEVTHQGLSSPV |
| | | | TKSFNRGEC |
| A | VH CDR1 | 3367 | SYWIG |
| (see, e.g., | VH CDR2 | 3368 | IIYPGDSDTRYSPSFQGQ |
| WO2021154477) | VH CDR3 | 3369 | FPYDSSGYYSFDY |
| | VL CDR1 | 3370 | RASQSISSYLN |
| | VL CDR2 | 3371 | AASSLQS |
| | VL CDR3 | 3372 | QQSYSTPLT |
| | VH | 3373 | QVQLVQSGAEVKKPGESLKISCKGSGYSFTSYWIGWVRQMP |
| | | | GKGLEWMGIIYPGDSDTRYSPSFQGQVTISADKSISTAYLQ |
| | | | WSSLKASDTAMYYCARFPYDSSGYYSFDYWGQGTLVTVSS |
| | VL | 3374 | DIQMTQSPSSLSASVGDRVTITCRASQSISSYLNWYQQKPG |
| | | | KAPKLLIYAASSLQSGVPSRFSGSGSGTDFTLTISSLQPED |
| | | | FATYYCQQSYSTPLTFGGGTKVEIK |
| B | VH CDR1 | 3375 | DDYMY |
| (see, e.g., | VH CDR2 | 3376 | WIDPENGDTEYASKFQD |
| WO2023283531) | VH CDR3 | 3377 | WLRRGLDY |
| | VL CDR1 | 3378 | RSSKSLLHSNGYTYLF |
| | VL CDR2 | 3379 | RMSNLAS |
| | VL CDR3 | 3380 | MQHLEYPFT |
| | VH murine | 3381 | EVQLQQSGAELVRPGASVKLSCTASGFNIKDDYMYWVKQRP |
| | | | EQGLEWIGWIDPENGDTEYASKFQDKATVTADTSSNTAYLQ |
| | | | LSSLTSEDTAVYYCTLWLRRGLDYWGQGTSVTVSS |
| | VH- | 3382 | EVQLVQSGSELKKPGASVKVSCTASGFNIKDDYMYWVRQPP |
| | Humanized | | GKGLEWIGWIDPENGDTEYASKFQDRVTVTADTSTNTAYME |
| | | | LSSLRSEDTAVYYCTLWLRRGLDYWGQGTLVTVSS |
| | VL | 3383 | DIVMTQAAPSVPVTPGESVSISCRSSKSLLHSNGYTYLFWF |
| | murine | | LQRPGQSPQLLIYRMSNLASGVPDRFSGSGSGTAFTLRISR |
| | | | VEAEDVGVYYCMQHLEYPFTFGGGTKLEIK |
| | VL- | 3384 | DIVMTQSPLSLPVTPGEPASISCRSSKSLLHSNGYTYLFWF |
| | Humanized | | QQRPGQSPRLLIYRMSNLASGVPDRFSGSGSGTDFTLKISR |
| | | | VEAEDVGVYYCMQHLEYPFTFGGGTKEIK |
| C | VH CDR1 | 3375 | DDYMY |
| (see, e.g., | VH CDR2 | 3385 | WIDPETGDTEYASKFQD |
| WO2023283531) | VH CDR3 | 3377 | WLRRGLDY |
| | VL CDR1 | 3378 | RSSKSLLHSNGYTYLF |
| | VL CDR2 | 3379 | RMSNLAS |
| | VL CDR3 | 3380 | MQHLEYPFT |
| | VH | 3386 | EVQLQQSGAELVRPGASVKLSCTASGFNIKDDYMYWVKQRP |
| | | | EQGLEWIGWIDPETGDTEYASKFQDKATVTADTSSNTAYLQ |
| | | | LSSLTSEDTAVYYCTLWLRRGLDYWGQGTSVTVSS |
| | VH- | 3387 | EVQLVQSGSELKKPGASVKVSCTASGFNIKDDYMYWVRQPP |
| | Humanized | | GKGLEWIGWIDPETGDTEYASKFQDRVTVTADTSTNTAYME |
| | | | LSSLRSEDTAVYYCTLWLRRGLDYWGQGTLVTVSS |
| | VL | 3383 | DIVMTQAAPSVPVTPGESVSISCRSSKSLLHSNGYTYLFWF |
| | | | LQRPGQSPQLLIYRMSNLASGVPDRFSGSGSGTAFTLRISR |
| | | | VEAEDVGVYYCMQHLEYPFTFGGGTKLEIK |
| | VL- | 3384 | DIVMTQSPLSLPVTPGEPASISCRSSKSLLHSNGYTYLFWF |
| | Humanized | | QQRPGQSPRLLIYRMSNLASGVPDRFSGSGSGTDFTLKISR |
| | | | VEAEDVGVYYCMQHLEYPFTFGGGTKEIK |
| D | VH CDR1 | 3375 | DDYMY |
| (see, e.g., | VH CDR2 | 3388 | WIDPESGDTEYASKFQD |
| WO2023283531) | VH CDR3 | 3377 | WLRRGLDY |
| | VL CDR1 | 3378 | RSSKSLLHSNGYTYLF |
| | VL CDR2 | 3379 | RMSNLAS |
| | VL CDR3 | 3380 | MQHLEYPFT |
| | VH | 3389 | EVQLQQSGAELVRPGASVKLSCTASGFNIKDDYMYWVKQRP |
| | | | EQGLEWIGWIDPESGDTEYASKFQDKATVTADTSSNTAYLQ |
| | | | LSSLTSEDTAVYYCTLWLRRGLDYWGQGTSVTVSS |
| | VH- | 3390 | EVQLVQSGSELKKPGASVKVSCTASGFNIKDDYMYWVRQPP |
| | Humanized | | GKGLEWIGWIDPESGDTEYASKFQDRVTVTADTSTNTAYME |
| | | | LSSLRSEDTAVYYCTLWLRRGLDYWGQGTLVTVSS |
| | VL | 3383 | DIVMTQAAPSVPVTPGESVSISCRSSKSLLHSNGYTYLFWF |
| | | | LQRPGQSPQLLIYRMSNLASGVPDRFSGSGSGTAFTLRISR |
| | | | VEAEDVGVYYCMQHLEYPFTFGGGTKLEIK |
| | VL- | 3384 | DIVMTQSPLSLPVTPGEPASISCRSSKSLLHSNGYTYLFWF |
| | Humanized | | QQRPGQSPRLLIYRMSNLASGVPDRFSGSGSGTDFTLKISR |
| | | | VEAEDVGVYYCMQHLEYPFTFGGGTKEIK |
| E | VH CDR1 | 3391 | SGYYWN |
| (see, e.g., | VH CDR2 | 3392 | YITFDGANNYNPSLKN |
| WO2023283531) | VH CDR3 | 3393 | SSYDYDVLDY |
| | VL CDR1 | 3394 | RASQDISNFLN |
| | VL CDR2 | 3395 | YTSRLHS |
| | VL CDR3 | 3396 | QQGHTLPYT |
| | VH | 3397 | DVQLQESGPGLVKPSQSLSLTCSVTGYSITSGYYWNWIRQF |
| | | | PGNKLEWMGYITFDGANNYNPSLKNRISITRDTSKNQFFLK |
| | | | LTSVTTEDTATYYCTRSSYDYDVLDYWGQGTTLTVSS |
| | VH- | 3398 | QVQLQESGPGLVKPSQTLSLTCSVTGYSITSGYYWNWIRQP |
| | Humanized 1 | | PGKGLEWMGYITFDGANNYNPSLKNRVSISRDTSKNQFSLK |
| | | | LSSVTAEDTATYYCTRSSYDYDVLDYWGQGTTVTVSS |
| | VH- | 3399 | QVQLQESGPGLVKPSQTLSLTCTVTGYSITSGYYWNWIRQP |
| | Humanized 2 | | PGKGLEWIGYITFDGANNYNPSLKNRVSISRDTSKNQFSLK |
| | | | LSSVTAEDTATYYCTRSSYDYDVLDYWGQGTTVTVSS |
| | VL | 3400 | DIQMTQTTSSLSASLGDRVTISCRASQDISNFLNWYQQRPD |
| | | | GTVKLLIYYTSRLHSGVPSRFSGSGSGTDFSLTVSNLEQED |
| | | | IATYFCQQGHTLPYTFGGGTKLEIK |
| | VL- | 3401 | DIQMTQSPSSLSASVGDRVTITCRASQDISNFLNWYQQKPG |
| | Humanized 1 | | QPVKLLIYYTSRLHSGVPSRFSGSGSGTDFTLTISSLQPED |
| | | | FATYFCQQGHTLPYTFGQGTKLEIK |
| | VL- | 3402 | DIQMTQSPSSLSASVGDRVTITCRASQDISNFLNWYQQKPG |
| | Humanized 2 | | QPVKLLIYYTSRLHSGVPSRFSGSGSGTDFTLTISSLQPED |
| | | | FATYYCQQGHTLPYTFGQGTKLEIK |
| F | VH CDR1 | 3403 | DYCIN |
| (see, e.g., | VH CDR2 | 3404 | WIYPGSGNTRYSERFKG |
| WO2023283531) | VH CDR3 | 3405 | EDYYPYHGMDY |
| | VL CDR1 | 3406 | RASESVDGYDNSFMH |
| | VL CDR2 | 3407 | RASNLES |
| | VL CDR3 | 3408 | QQSSEDPWT |
| | VH | 3409 | QIQLQQSGPELVRPGASVKISCKASGYSFTDYCINWVNQRP |
| | | | GQGLEWIGWIYPGSGNTRYSERFKGKATLTVDTSSNTAYMQ |
| | | | LSSLTSEDSAVYFCAREDYYPYHGMDYWGQGTSVTVSS |
| | VL | 3410 | DIVLTQSPTSLAVSLGQRATISCRASESVDGYDNSFMHWYQ |
| | | | QKPGQPPKLLIFRASNLESGIPARFSGSGSRTDFTLTINPV |
| | | | EAADVATYYCQQSSEDPWTFGGGTKLEIK |
| G | VH CDR1 | 3411 | DYYIN |
| (see, e.g., | VH CDR2 | 3404 | WIYPGSGNTRYSERFKG |
| WO2023283531) | VH CDR3 | 3405 | EDYYPYHGMDY |
| | VL CDR1 | 3406 | RASESVDGYDNSFMH |
| | VL CDR2 | 3407 | RASNLES |
| | VL CDR3 | 3408 | QQSSEDPWT |
| | VH | 3412 | QIQLQQSGPELVRPGASVKISCKASGYSFTDYYINWVNQRP |
| | | | GQGLEWIGWIYPGSGNTRYSERFKGKATLTVDTSSNTAYMQ |
| | | | LSSLTSEDSAVYFCAREDYYPYHGMDYWGQGTSVTVSS |
| | VL | 3410 | DIVLTQSPTSLAVSLGQRATISCRASESVDGYDNSFMHWYQ |
| | | | QKPGQPPKLLIFRASNLESGIPARFSGSGSRTDFTLTINPV |
| | | | EAADVATYYCQQSSEDPWTFGGGTKLEIK |
| H | VH CDR1 | 3413 | DYDIN |
| (see, e.g., | VH CDR2 | 3404 | WIYPGSGNTRYSERFKG |
| WO2023283531) | VH CDR3 | 3405 | EDYYPYHGMDY |
| | VL CDR1 | 3406 | RASESVDGYDNSFMH |
| | VL CDR2 | 3407 | RASNLES |
| | VL CDR3 | 3408 | QQSSEDPWT |
| | VH | 3414 | QIQLQQSGPELVRPGASVKISCKASGYSFTDYDINWVNQRP |
| | | | GQGLEWIGWIYPGSGNTRYSERFKGKATLTVDTSSNTAYMQ |
| | | | LSSLTSEDSAVYFCAREDYYPYHGMDYWGQGTSVTVSS |
| | VL | 3410 | DIVLTQSPTSLAVSLGQRATISCRASESVDGYDNSFMHWYQ |
| | | | QKPGQPPKLLIFRASNLESGIPARFSGSGSRTDFTLTINPV |
| | | | EAADVATYYCQQSSEDPWTFGGGTKLEIK |
| H | VH CDR1 | 3415 | NYYMH |
| (see, e.g., | VH CDR2 | 3416 | WIYPGDGNTHYNEKFKG |
| US20190240346) | VH CDR3 | 3417 | DNSGYVGFPY |
| | VL CDR1 | 3418 | KSSQSLFNSANQENYLT |
| | VL CDR2 | 3419 | WASTRDS |
| | VL CDR3 | 3420 | QNDYSYPLT |
| | VH | 3421 | EVQLQQSGPELVKPGTSMNISCKASGYTFTNYYMHWVRQRP |
| | | | GQGLEWIGWIYPGDGNTHYNEKFKGKTTLTADTSSITAYML |
| | | | LSSLTSEDSAVYFCARDNSGYVGFPYWGQGTTLTVSS |
| | VL | 3422 | MDIKMTQSPSSLTVTAGERVTMSCKSSQSLFNSANQENYLT |
| | | | WYQQRPGQPPKLLIYWASTRDSGVPDRFTGSGSGTDFPLTI |
| | | | SSVQAEDLAVYYCQNDYSYPLTFGAGTKLEIK |
| I | VH CDR1 | 3423 | SYAMS |
| (see, e.g., | VH CDR2 | 3424 | YIWSGGSTDYASWAKS |
| US11584793) | VH CDR3 | 3425 | RYGTSYPDYGDASGFDP |
| | VL CDR1 | 3426 | RASQSISSYLA |
| | VL CDR2 | 3427 | RASTLAS |
| | VL CDR3 | 3428 | QQNYASSNVDNT |
| | VH | 3429 | QSMQESGPGLVKPSQTLSLTCTVSGFSLSSYAMSWIRQHPG |
| | | | KGLEWIGYIWSGGSTDYASWAKSRVTISKTSTTVSLKLSSV |
| | | | TAADTAVYYCARRYGTSYPDYGDASGFDPWGQGTLVTVSS |
| | VL | 3430 | AIQLTQSPSSLSASVGDRVTITCRASQSISSYLAWYQQKPG |
| | | | KAPKLLIYRASTLASGVPSRFSGSGSGTDFTLTISSLQPED |
| | | | FATYYCQQNYASSNVDNTFGGGTKVEIK |
| J | VH CDR1 | 3431 | NSYWIC |
| (see, e.g., | VH CDR2 | 3432 | CINTDADSTNYASWARG |
| WO2023034409) | VH CDR3 | 3433 | QNNVFDPGYNL |
| | VL CDR1 | 3434 | RASQNIGSNLA |
| | VL CDR2 | 3435 | DASKLES |
| | VL CDR3 | 3436 | QCTVRGGAYGNA |
| | VH | 3437 | QSLVESGGGVVQPGRSLRLSCKASGFSFSNSYWICWVRQAP |
| | | | GKGLEWVGCINTDADSTNYASWARGRFTISKTSSTTVYLQM |
| | | | NSLRAEDTAVYYCARQNNVFDPGYNLWGPGTLVTVSS |
| | VL | 3438 | ELVLTQSPSTLSASVGDRVTITCRASQNIGSNLAWYQQKPG |
| | | | KAPKLLIYDASKLESGVPSRFSGSGSGTEFTLTISSLQPDD |
| | | | FATYYCQCTVRGGAYGNAFGGGTKVEIK |
| K | VH CDR1 | 3354 | SYWMH |
| (see, e.g., | VH CDR2 | 3355 | EINPINGRTNYIEKFKS |
| WO2016081643) | VH CDR3 | 3356 | GTRAYHY |
| | VL CDR1 | 3357 | RASDNLYSNLA |
| | VL CDR2 | 3358 | DATNLAD |
| | VL CDR3 | 3359 | QHFWGTPLT |
| | VH | 3439 | QVQLQQPGAELVKPGASVKLSCKASGYTFTSYWMHWVKQRP |
| | | | GQGLEWIGEINPINGRTNYIEKFKSKATLTVDKSSSTAYMQ |
| | | | LSSLTSEDSAVYYCARGTRAYHYWGQGTSVTVSS |
| | VL | 3440 | DIQMTQSPASLSVSVGETVTITCRASDNLYSNLAWYQQKQG |
| | | | KSPQLLVYDATNLADGVPSRFSGSGSGTQYSLKINSLQSED |
| | | | FGTYYCQHFWGTPLTFGAGTKLELK |
| L | VH CDR1 | 3441 | NYWMH |
| (see, e.g., | VH CDR2 | 3442 | EINPINGRSNYAQKFQG |
| WO2020132584) | VH CDR3 | 3443 | GTRAMHY |
| | VL CDR1 | 3444 | RTSENIYNNLA |
| | VL CDR2 | 3445 | AATNLAD |
| | VL CDR3 | 3359 | QHFWGTPLT |
| | VH | 3446 | QVQLVQSGAEVKKPGASVKVSCKASGYTFTNYWMHWVRQAP |
| | | | GQGLEWMGEINPINGRSNYAQKFQGRVTLTVDTSISTAYME |
| | | | LSRLRSDDTAVYYCARGTRAMHYWGQGTLVTVSS |
| | VL | 3447 | DIQMTQSPSSLSASVGDRVTITCRTSENIYNNLAWYQQKPG |
| | | | KSPKLLIYAATNLADGVPSRFSGSGSGTDYTLTISSLQPED |
| | | | FATYYCQHFWGTPLTFGGGTKVEIK |
| M | VH CDR1 | 3441 | NYWMH |
| (see, e.g., | VH CDR2 | 3448 | EINPINGRSNYAEKFQG |
| WO2020132584) | VH CDR3 | 3443 | GTRAMHY |
| | VL CDR1 | 3444 | RTSENIYNNLA |
| | VL CDR2 | 3445 | AATNLAD |
| | VL CDR3 | 3359 | QHFWGTPLT |
| | VH | 3446 | QVQLVQSGAEVKKPGASVKVSCKASGYTFTNYWMHWVRQAP |
| | | | GQGLEWIGEINPINGRSNYAEKFQGRVTLTVDTSSSTAYME |
| | | | LSRLRSDDTAVYYCARGTRAMHYWGQGTLVTVSS |
| | VL | 3447 | DIQMTQSPSSLSASVGDRVTITCRTSENIYNNLAWYQQKPG |
| | | | KAPKLLIYAATNLADGVPSRFSGSGSGTDYTLTISSLQPED |
| | | | FATYYCQHFWGTPLTFGGGTKVEIK |
| N | VH CDR1 | 3441 | NYWMH |
| (see, e.g., | VH CDR2 | 3448 | EINPIQGRSNYAEKFQG |
| WO2020132584) | VH CDR3 | 3443 | GTRAMHY |
| | VL CDR1 | 3444 | RTSENIYNNLA |
| | VL CDR2 | 3449 | AATNLAE |
| | VL CDR3 | 3450 | QHFWGTPLTF |
| | VH | 3451 | QVQLVQSGAEVKKPGASVKVSCKASGYTFTNYWMHWVRQAP |
| | | | GQGLEWMGEINPIQGRSNYAEKFQGRVTLTVDTSSSTAYME |
| | | | LSSLRSEDTATYYCARGTRAMHYWGQGTLVTVSS |
| | VL | 3452 | DIQMTQSPSSLSASVGDRVTITCRTSENIYNNLAWYQQKPG |
| | | | KAPKLLIYAATNLAEGVPSRFSGSGSGTDYTLTISSLQPED |
| | | | FATYYCQHFWGTPLTFGGGTKVEIK |
| P | VH CDR1 | 3354 | SYWMH |
| (see, e.g., | VH CDR2 | 3453 | AIYPGNSETGYAQKFQG |
| US11267896B2) | VH CDR3 | 3454 | ENWDPGFAF |
| | VL CDR1 | 3455 | SASSSVYYM |
| | VL CDR2 | 3456 | STSNLAS |
| | VL CDR3 | 3457 | QQRRNYPYT |
| | VH | 3458 | QVQLVQSGAEVKKPGASVKMSCKASGYTFTSYWMHWVRQAP |
| | | | GQGLEWIGAIYPGNSETGYAQKFQGRATLTADTSTSTAYME |
| | | | LSSLRSEDTAVYYCTRENWDPGFAFWGQGTLITVSS |
| | VL | 3459 | DIQMTQSPSSLSASVGDRVTITCSASSSVYYMYWFQQKPGK |
| | | | APKLWIYSTSNLASGVPSRFSGSGSGTDYTLTISSMQPEDE |
| | | | ATYYCQQRRNYPYTFGQGTKLEIK |
| Q | VH CDR1 | 3354 | SYWMH |
| (see, e.g., | VH CDR2 | 3355 | EINPINGRTNYIEKFKS |
| US20220143206) | VH CDR3 | 3356 | GTRAYHY |
| | VL CDR1 | 3357 | RASDNLYSNLA |
| | VL CDR2 | 3358 | DATNLAD |
| | VL CDR3 | 3359 | QHFWGTPLT |
| | VH-1 | 3439 | QVQLQQPGAELVKPGASVKLSCKASGYTFTSYWMHWVKQRP |
| | | | GQGLEWIGEINPTNGRTNYIEKFKSKATLTVDKSSSTAYMQ |
| | | | LSSLTSEDSAVYYCARGTRAYHYWGQGTSVTVSS |
| | VL-1 | 3440 | DIQMTQSPASLSVSVGETVTITCRASDNLYSNLAWYQQKQG |
| | | | KSPQLLVYDATNLADGVPSRFSGSGSGTQYSLKINSLQSED |
| | | | FGTYYCQHFWGTPLTFGAGTKLELK |
| | VH-1 | 3360 | EVQLVQSGAEVKKPGASVKVSCKASGYTFTSYWMHWVRQAP |
| | | | GQRLEWIGEINPINGRTNYIEKFKSRATLTVDKSASTAYME |
| | | | LSSLRSEDTAVYYCARGTRAYHYWGQGTMVTVSS |
| | VL-1 | 3460 | DIQMTSPSSLSASVGDRVTITCRASDNLYSNLAWYQQKPGK |
| | | | SPKLLVYDATNLADGVPSRFSGSGSGTDYSLKINSLQSEDF |
| | | | GTYYCQHFWGTPLTFGAGTKLELK |
| S | VH CDR1 | 3441 | NYWMH |
| | VH CDR2 | 3448 | EINPINGRSNYAEKFQG |
| | VH CDR3 | 3443 | GTRAMHY |
| | VL CDR1 | 3444 | RTSENIYNNLA |
| | VL CDR2 | 3445 | AATNLAD |
| | VL CDR3 | 3359 | QHFWGTPLT |
| | VH | 3446 | QVQLVQSGAEVKKPGASVKVSCKASGYTFTNYWMHWVRQAP |
| | | | GQGLEWIGEINPINGRSNYAEKFQGRVTLTVDTSSSTAYME |
| | | | LSRLRSDDTAVYYCARGTRAMHYWGQGTLVTVSS |
| | VL | 3447 | DIQMTQSPSSLSASVGDRVTITCRISENIYNNLAWYQQKPG |
| | | | KSPKLLIYAATNLADGVPSRFSGSGSGTDYTLTISSLQPED |
| | | | FATYYCQHFWGTPLTFGGGTKVEIK |

In some embodiments, the anti-TFR (e.g., hTFR, e.g., hTFR1) antibody comprises an anti-TFR (e.g., hTFR, e.g., hTFR1) antibody named and/or incorporated by reference herein).

In some embodiments, the anti-TFR (e.g., hTFR, e.g., hTFR1) comprises a VH that comprises: a VH CDR1, a VH CDR2, and a VH CDR3.

In some embodiments, the amino acid sequence of VH CDR1 comprises or consists of the amino acid sequence of a VH CDR1 of a VH of an anti-TFR antibody named and/or incorporated by reference herein), or the amino acid sequence of a VH CDR1 of an anti-TFR antibody named and/or incorporated by reference herein) comprising or consisting of 1, 2, or 3 amino acid variations (e.g., substitution, deletion, addition, etc.). In some embodiments, the amino acid sequence of VH CDR2 comprises or consists of the amino acid sequence of a VH CDR2 of a VH of an anti-TFR antibody named and/or incorporated by reference herein), or the amino acid sequence of a VH CDR2 of a VH of an anti-TFR antibody named and/or incorporated by reference herein) comprising or consisting of 1, 2, or 3 amino acid variations (e.g., substitution, deletion, addition, etc.). In some embodiments, the amino acid sequence of VH CDR3 comprises or consists of the amino acid sequence of a VH CDR3 of a VH of an anti-TFR antibody named and/or incorporated by reference herein), or the amino acid sequence of a VH CDR3 of a VH of an anti-TFR antibody named and/or incorporated by reference herein) comprising or consisting of 1, 2, or 3 amino acid variations (e.g., substitution, deletion, addition, etc.).

In some embodiments, the amino acid sequence of VH CDR1 comprises or consists of the amino acid sequence of a VH CDR1 of a VH of an anti-TFR antibody named and/or incorporated by reference herein), or the amino acid sequence of a VH CDR1 of an anti-TFR antibody named and/or incorporated by reference herein) comprising or consisting of 1, 2, or 3 amino acid variations (e.g., substitution, deletion, addition, etc.); the amino acid sequence of VH CDR2 comprises or consists of the amino acid sequence of a VH CDR2 of a VH of an anti-TFR antibody named and/or incorporated by reference herein), or the amino acid sequence of a VH CDR2 of a VH of an anti-TFR antibody named and/or incorporated by reference herein) comprising or consisting of 1, 2, or 3 amino acid variations (e.g., substitution, deletion, addition, etc.); and the amino acid sequence of VH CDR3 comprises or consists of the amino acid sequence of a VH CDR3 of a VH of an anti-TFR antibody named and/or incorporated by reference herein), or the amino acid sequence of a VH CDR3 of a VH of an anti-TFR antibody named and/or incorporated by reference herein) comprising or consisting of 1, 2, or 3 amino acid variations (e.g., substitution, deletion, addition, etc.).

In some embodiments, the amino acid sequence of VH CDR1 comprises or consists of the amino acid sequence of a VH CDR1 of an anti-TFR antibody named and/or incorporated by reference herein), or the amino acid sequence of a VH CDR1 of an anti-TFR antibody named and/or incorporated by reference herein) comprising or consisting of 1, 2, or 3 amino acid variations (e.g., substitution, deletion, addition, etc.). In some embodiments, the amino acid sequence of VH CDR2 comprises or consists of the amino acid sequence of a VH CDR2 of an anti-TFR antibody named and/or incorporated by reference herein), or the amino acid sequence of a VH CDR2 of an anti-TFR antibody named and/or incorporated by reference herein) comprising or consisting of 1, 2, or 3 amino acid variations (e.g., substitution, deletion, addition, etc.). In some embodiments, the amino acid sequence of VH CDR3 comprises or consists of the amino acid sequence of a VH CDR3 of an anti-TFR antibody named and/or incorporated by reference herein), or the amino acid sequence of a VH CDR3 of an anti-TFR antibody named and/or incorporated by reference herein) comprising or consisting of 1, 2, or 3 amino acid variations (e.g., substitution, deletion, addition, etc.).

In some embodiments, the amino acid sequence of VH CDR1 comprises or consists of the amino acid sequence of a VH CDR1 of an anti-TFR antibody named and/or incorporated by reference herein), or the amino acid sequence of a VH CDR1 of an anti-TFR antibody named and/or incorporated by reference herein) comprising or consisting of 1, 2, or 3 amino acid variations (e.g., substitution, deletion, addition, etc.); the amino acid sequence of VH CDR2 comprises or consists of the amino acid sequence of a VH CDR2 of an anti-TFR antibody named and/or incorporated by reference herein), or the amino acid sequence of a VH CDR2 of an anti-TFR antibody named and/or incorporated by reference herein) comprising or consisting of 1, 2, or 3 amino acid variations (e.g., substitution, deletion, addition, etc.); and the amino acid sequence of VH CDR3 comprises or consists of the amino acid sequence of a VH CDR3 of an anti-TFR antibody named and/or incorporated by reference herein), or the amino acid sequence of a VH CDR3 of an anti-TFR antibody named and/or incorporated by reference herein) comprising or consisting of 1, 2, or 3 amino acid variations (e.g., substitution, deletion, addition, etc.).

In some embodiments, the anti-TFR (e.g., hTFR, e.g., hTFR1) comprises a VL that comprises: VL CDR1, VL CDR2, and VL CDR3.

In some embodiments, the amino acid sequence of VL CDR1 comprises or consists of the amino acid sequence of a VL CDR1 of a VL of an anti-TFR antibody named and/or incorporated by reference herein), or the amino acid sequence of a VL CDR1 of a VL of an anti-TFR antibody named and/or incorporated by reference herein) comprising or consisting of 1, 2, or 3 amino acid variations (e.g., substitution, deletion, addition, etc.). In some embodiments, the amino acid sequence of VL CDR2 comprises or consists of the amino acid sequence of a VL CDR2 of a VL of an anti-TFR antibody named and/or incorporated by reference herein), or the amino acid sequence of a VL CDR2 of a VL of an anti-TFR antibody named and/or incorporated by reference herein) comprising or consisting of 1, 2, or 3 amino acid variations (e.g., substitution, deletion, addition, etc.). In some embodiments, the amino acid sequence of VL CDR3 comprises or consists of the amino acid sequence of a VL CDR3 of a VL of an anti-TFR antibody named and/or incorporated by reference herein), or the amino acid sequence of a VL CDR3 of a VL of an anti-TFR antibody named and/or incorporated by reference herein) comprising or consisting of 1, 2, or 3 amino acid variations (e.g., substitution, deletion, addition, etc.).

In some embodiments, the amino acid sequence of VL CDR1 comprises or consists of the amino acid sequence of a VL CDR1 of a VL of an anti-TFR antibody named and/or incorporated by reference herein), or the amino acid sequence of a VL CDR1 of a VL of an anti-TFR antibody named and/or incorporated by reference herein) comprising or consisting of 1, 2, or 3 amino acid variations (e.g., substitution, deletion, addition, etc.); the amino acid sequence of VL CDR2 comprises or consists of the amino acid sequence of a VL CDR2 of a VL of an anti-TFR antibody named and/or incorporated by reference herein), or the amino acid sequence of a VL CDR2 of a VL of an anti-TFR antibody named and/or incorporated by reference herein) comprising or consisting of 1, 2, or 3 amino acid variations (e.g., substitution, deletion, addition, etc.); and the amino acid sequence of VL CDR3 comprises or consists of the amino acid sequence of a VL CDR3 of a VL of an anti-TFR antibody named and/or incorporated by reference herein), or the amino acid sequence of a VL CDR3 of a VL of an anti-TFR antibody named and/or incorporated by reference herein) comprising or consisting of 1, 2, or 3 amino acid variations (e.g., substitution, deletion, addition, etc.).

In some embodiments, the amino acid sequence of VL CDR1 comprises or consists of the amino acid sequence of a VL CDR1 of an anti-TFR antibody named and/or incorporated by reference herein), or the amino acid sequence of a VL CDR1 of an anti-TFR antibody named and/or incorporated by reference herein) comprising or consisting of 1, 2, or 3 amino acid variations (e.g., substitution, deletion, addition, etc.). In some embodiments, the amino acid sequence of VL CDR2 comprises or consists of the amino acid sequence of a VL CDR2 of an anti-TFR antibody named and/or incorporated by reference herein), or the amino acid sequence of a VL CDR2 of an anti-TFR antibody named and/or incorporated by reference herein) comprising or consisting of 1, 2, or 3 amino acid variations (e.g., substitution, deletion, addition, etc.). In some embodiments, the amino acid sequence of VL CDR3 comprises or consists of the amino acid sequence of a VL CDR3 of an anti-TFR antibody named and/or incorporated by reference herein), or the amino acid sequence of a VL CDR3 of an anti-TFR antibody named and/or incorporated by reference herein) comprising or consisting of 1, 2, or 3 amino acid variations (e.g., substitution, deletion, addition, etc.).

In some embodiments, the amino acid sequence of VL CDR1 comprises or consists of the amino acid sequence of a VL CDR1 of an anti-TFR antibody named and/or incorporated by reference herein), or the amino acid sequence of a VL CDR1 of an anti-TFR antibody named and/or incorporated by reference herein) comprising or consisting of 1, 2, or 3 amino acid variations (e.g., substitution, deletion, addition, etc.); the amino acid sequence of VL CDR2 comprises or consists of the amino acid sequence of a VL CDR2 of an anti-TFR antibody named and/or incorporated by reference herein), or the amino acid sequence of a VL CDR2 of an anti-TFR antibody named and/or incorporated by reference herein) comprising or consisting of 1, 2, or 3 amino acid variations (e.g., substitution, deletion, addition, etc.); and the amino acid sequence of VL CDR3 comprises or consists of the amino acid sequence of a VL CDR3 of an anti-TFR antibody named and/or incorporated by reference herein), or the amino acid sequence of a VL CDR3 of an anti-TFR antibody named and/or incorporated by reference herein) comprising or consisting of 1, 2, or 3 amino acid variations (e.g., substitution, deletion, addition, etc.).

In some embodiments, the anti-TFR (e.g., hTFR, e.g., hTFR1) comprises a VH that comprises: VH CDR1, VH CDR2, and VH CDR3; and a VL that comprises: VL CDR1, VL CDR2, and VL CDR3.

In some embodiments, the amino acid sequence of VH CDR1 comprises or consists of the amino acid sequence of a VH CDR1 of a VH of an anti-TFR antibody named and/or incorporated by reference herein), or the amino acid sequence of a VH CDR1 of a VH of an anti-TFR antibody named and/or incorporated by reference herein) comprising or consisting of 1, 2, or 3 amino acid variations (e.g., substitution, deletion, addition, etc.); the amino acid sequence of VH CDR2 comprises or consists of the amino acid sequence of a VH CDR2 of a VH of an anti-TFR antibody named and/or incorporated by reference herein), or the amino acid sequence of a VH CDR2 of a VH of an anti-TFR antibody named and/or incorporated by reference herein) comprising or consisting of 1, 2, or 3 amino acid variations (e.g., substitution, deletion, addition, etc.); the amino acid sequence of VH CDR3 comprises or consists of the amino acid sequence of a VH CDR3 of a VH of an anti-TFR antibody named and/or incorporated by reference herein), or the amino acid sequence of a VH CDR3 of a VH of an anti-TFR antibody named and/or incorporated by reference herein) comprising or consisting of 1, 2, or 3 amino acid variations (e.g., substitution, deletion, addition, etc.); the amino acid sequence of VL CDR1 comprises or consists of the amino acid sequence of a VL CDR1 of a VL of an anti-TFR antibody named and/or incorporated by reference herein), or the amino acid sequence of a VL CDR1 of a VL of an anti-TFR antibody named and/or incorporated by reference herein) comprising or consisting of 1, 2, or 3 amino acid variations (e.g., substitution, deletion, addition, etc.); the amino acid sequence of VL CDR2 comprises or consists of the amino acid sequence of a VL CDR2 of a VL of an anti-TFR antibody named and/or incorporated by reference herein), or the amino acid sequence of a VL CDR2 of a VL of an anti-TFR antibody named and/or incorporated by reference herein) comprising or consisting of 1, 2, or 3 amino acid variations (e.g., substitution, deletion, addition, etc.); and the amino acid sequence of VL CDR3 comprises or consists of the amino acid sequence of a VL CDR3 of a VL of an anti-TFR antibody named and/or incorporated by reference herein), or the amino acid sequence of a VL CDR3 of a VL of an anti-TFR antibody named and/or incorporated by reference herein) comprising or consisting of 1, 2, or 3 amino acid variations (e.g., substitution, deletion, addition, etc.).

In some embodiments, the amino acid sequence of VH CDR1 comprises or consists of the amino acid sequence of a VH CDR1 of an anti-TFR antibody named and/or incorporated by reference herein), or the amino acid sequence of a VH CDR1 of an anti-TFR antibody named and/or incorporated by reference herein) comprising or consisting of 1, 2, or 3 amino acid variations (e.g., substitution, deletion, addition, etc.); the amino acid sequence of VH CDR2 comprises or consists of the amino acid sequence of a VH CDR2 of an anti-TFR antibody named and/or incorporated by reference herein), or the amino acid sequence of a VH CDR2 of an anti-TFR antibody named and/or incorporated by reference herein) comprising or consisting of 1, 2, or 3 amino acid variations (e.g., substitution, deletion, addition, etc.); the amino acid sequence of VH CDR3 comprises or consists of the amino acid sequence of a VH CDR3 of an anti-TFR antibody named and/or incorporated by reference herein), or the amino acid sequence of a VH CDR3 of an anti-TFR antibody named and/or incorporated by reference herein) comprising or consisting of 1, 2, or 3 amino acid variations (e.g., substitution, deletion, addition, etc.); the amino acid sequence of VL CDR1 comprises or consists of the amino acid sequence of a VL CDR1 of an anti-TFR antibody named and/or incorporated by reference herein), or the amino acid sequence CDR1 of a VL of an anti-TFR antibody named and/or incorporated by reference herein) comprising or consisting of 1, 2, or 3 amino acid variations (e.g., substitution, deletion, addition, etc.); the amino acid sequence of VL CDR2 comprises or consists of the amino acid sequence of a VL CDR2 of an anti-TFR antibody named and/or incorporated by reference herein), or the amino acid sequence of a VL CDR2 of an anti-TFR antibody named and/or incorporated by reference herein) comprising or consisting of 1, 2, or 3 amino acid variations (e.g., substitution, deletion, addition, etc.); and the amino acid sequence of VL CDR3 comprises or consists of the amino acid sequence of a VL CDR3 of an anti-TFR antibody named and/or incorporated by reference herein), or the amino acid sequence of a VL CDR3 of an anti-TFR antibody named and/or incorporated by reference herein) comprising or consisting of 1, 2, or 3 amino acid variations (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH of an anti-TFR antibody named and/or incorporated by reference herein). In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL of an anti-TFR antibody named and/or incorporated by reference herein). In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH of an anti-TFR antibody named and/or incorporated by reference herein); and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL of an anti-TFR antibody named and/or incorporated by reference herein).

In some embodiments, the anti-TFR (e.g., hTFR, e.g., hTFR1) antibody comprises an anti-TFR (e.g., hTFR, e.g., hTFR1) antibody provided in Table 14.

In some embodiments, the anti-TFR (e.g., hTFR, e.g., hTFR1) comprises a VH that comprises: a VH CDR1, a VH CDR2, and a VH CDR3.

In some embodiments, the amino acid sequence of VH CDR1 comprises or consists of the amino acid sequence of a VH CDR1 of a VH set forth in Table 14, or the amino acid sequence of a VH CDR1 of a VH set forth in Table 14 comprising or consisting of 1, 2, or 3 amino acid variations (e.g., substitution, deletion, addition, etc.). In some embodiments, the amino acid sequence of VH CDR2 comprises or consists of the amino acid sequence of a VH CDR2 of a VH set forth in Table 14, or the amino acid sequence of a VH CDR2 of a VH set forth in Table 14 comprising or consisting of 1, 2, or 3 amino acid variations (e.g., substitution, deletion, addition, etc.). In some embodiments, the amino acid sequence of VH CDR3 comprises or consists of the amino acid sequence of a VH CDR3 of a VH set forth in Table 14, or the amino acid sequence of a VH CDR3 of a VH set forth in Table 14 comprising or consisting of 1, 2, or 3 amino acid variations (e.g., substitution, deletion, addition, etc.).

In some embodiments, the amino acid sequence of VH CDR1 comprises or consists of the amino acid sequence of a VH CDR1 of a VH set forth in Table 14, or the amino acid sequence of a VH CDR1 of a VH set forth in Table 14 comprising or consisting of 1, 2, or 3 amino acid variations (e.g., substitution, deletion, addition, etc.); the amino acid sequence of VH CDR2 comprises or consists of the amino acid sequence of a VH CDR2 of a VH set forth in Table 14, or the amino acid sequence of a VH CDR2 of a VH set forth in Table 14 comprising or consisting of 1, 2, or 3 amino acid variations (e.g., substitution, deletion, addition, etc.); and the amino acid sequence of VH CDR3 comprises or consists of the amino acid sequence of a VH CDR3 of a VH set forth in Table 14, or the amino acid sequence of a VH CDR3 of a VH set forth in Table 14 comprising or consisting of 1, 2, or 3 amino acid variations (e.g., substitution, deletion, addition, etc.).

In some embodiments, the amino acid sequence of VH CDR1 comprises or consists of the amino acid sequence of a VH CDR1 set forth in Table 14, or the amino acid sequence of a VH CDR1 set forth in Table 14 comprising or consisting of 1, 2, or 3 amino acid variations (e.g., substitution, deletion, addition, etc.). In some embodiments, the amino acid sequence of VH CDR2 comprises or consists of the amino acid sequence of a VH CDR2 set forth in Table 14, or the amino acid sequence of a VH CDR2 set forth in Table 14 comprising or consisting of 1, 2, or 3 amino acid variations (e.g., substitution, deletion, addition, etc.). In some embodiments, the amino acid sequence of VH CDR3 comprises or consists of the amino acid sequence of a VH CDR3 set forth in Table 14, or the amino acid sequence of a VH CDR3 set forth in Table 14 comprising or consisting of 1, 2, or 3 amino acid variations (e.g., substitution, deletion, addition, etc.).

In some embodiments, the amino acid sequence of VH CDR1 comprises or consists of the amino acid sequence of a VH CDR1 set forth in Table 14, or the amino acid sequence of a VH CDR1 set forth in Table 14 comprising or consisting of 1, 2, or 3 amino acid variations (e.g., substitution, deletion, addition, etc.); the amino acid sequence of VH CDR2 comprises or consists of the amino acid sequence of a VH CDR2 set forth in Table 14, or the amino acid sequence of a VH CDR2 set forth in Table 14 comprising or consisting of 1, 2, or 3 amino acid variations (e.g., substitution, deletion, addition, etc.); and the amino acid sequence of VH CDR3 comprises or consists of the amino acid sequence of a VH CDR3 set forth in Table 14, or the amino acid sequence of a VH CDR3 set forth in Table 14 comprising or consisting of 1, 2, or 3 amino acid variations (e.g., substitution, deletion, addition, etc.).

In some embodiments, the anti-TFR (e.g., hTFR, e.g., hTFR1) comprises a VL that comprises: VL CDR1, VL CDR2, and VL CDR3.

In some embodiments, the amino acid sequence of VL CDR1 comprises or consists of the amino acid sequence of a VL CDR1 of a VL set forth in Table 14, or the amino acid sequence of a VL CDR1 of a VL set forth in Table 14 comprising or consisting of 1, 2, or 3 amino acid variations (e.g., substitution, deletion, addition, etc.). In some embodiments, the amino acid sequence of VL CDR2 comprises or consists of the amino acid sequence of a VL CDR2 of a VL set forth in Table 14, or the amino acid sequence of a VL CDR2 of a VL set forth in Table 14 comprising or consisting of 1, 2, or 3 amino acid variations (e.g., substitution, deletion, addition, etc.). In some embodiments, the amino acid sequence of VL CDR3 comprises or consists of the amino acid sequence of a VL CDR3 of a VL set forth in Table 14, or the amino acid sequence of a VL CDR3 of a VL set forth in Table 14 comprising or consisting of 1, 2, or 3 amino acid variations (e.g., substitution, deletion, addition, etc.).

In some embodiments, the amino acid sequence of VL CDR1 comprises or consists of the amino acid sequence of a VL CDR1 of a VL set forth in Table 14, or the amino acid sequence of a VL CDR1 of a VL set forth in Table 14 comprising or consisting of 1, 2, or 3 amino acid variations (e.g., substitution, deletion, addition, etc.); the amino acid sequence of VL CDR2 comprises or consists of the amino acid sequence of a VL CDR2 of a VL set forth in Table 14, or the amino acid sequence of a VL CDR2 of a VL set forth in Table 14 comprising or consisting of 1, 2, or 3 amino acid variations (e.g., substitution, deletion, addition, etc.); and the amino acid sequence of VL CDR3 comprises or consists of the amino acid sequence of a VL CDR3 of a VL set forth in Table 14, or the amino acid sequence of a VL CDR3 of a VL set forth in Table 14 comprising or consisting of 1, 2, or 3 amino acid variations (e.g., substitution, deletion, addition, etc.).

In some embodiments, the amino acid sequence of VL CDR1 comprises or consists of the amino acid sequence of a VL CDR1 set forth in Table 14, or the amino acid sequence of a VL CDR1 set forth in Table 14 comprising or consisting of 1, 2, or 3 amino acid variations (e.g., substitution, deletion, addition, etc.). In some embodiments, the amino acid sequence of VL CDR2 comprises or consists of the amino acid sequence of a VL CDR2 set forth in Table 14, or the amino acid sequence of a VL CDR2 set forth in Table 14 comprising or consisting of 1, 2, or 3 amino acid variations (e.g., substitution, deletion, addition, etc.). In some embodiments, the amino acid sequence of VL CDR3 comprises or consists of the amino acid sequence of a VL CDR3 set forth in Table 14, or the amino acid sequence of a VL CDR3 set forth in Table 14 comprising or consisting of 1, 2, or 3 amino acid variations (e.g., substitution, deletion, addition, etc.).

In some embodiments, the amino acid sequence of VL CDR1 comprises or consists of the amino acid sequence of a VL CDR1 set forth in Table 14, or the amino acid sequence of a VL CDR1 set forth in Table 14 comprising or consisting of 1, 2, or 3 amino acid variations (e.g., substitution, deletion, addition, etc.); the amino acid sequence of VL CDR2 comprises or consists of the amino acid sequence of a VL CDR2 set forth in Table 14, or the amino acid sequence of a VL CDR2 set forth in Table 14 comprising or consisting of 1, 2, or 3 amino acid variations (e.g., substitution, deletion, addition, etc.); and the amino acid sequence of VL CDR3 comprises or consists of the amino acid sequence of a VL CDR3 set forth in Table 14, or the amino acid sequence of a VL CDR3 set forth in Table 14 comprising or consisting of 1, 2, or 3 amino acid variations (e.g., substitution, deletion, addition, etc.).

In some embodiments, the anti-TFR (e.g., hTFR, e.g., hTFR1) comprises a VH that comprises: VH CDR1, VH CDR2, and VH CDR3; and a VL that comprises: VL CDR1, VL CDR2, and VL CDR3.

In some embodiments, the amino acid sequence of VH CDR1 comprises or consists of the amino acid sequence of a VH CDR1 of a VH set forth in Table 14, or the amino acid sequence of a VH CDR1 of a VH set forth in Table 14 comprising or consisting of 1, 2, or 3 amino acid variations (e.g., substitution, deletion, addition, etc.); the amino acid sequence of VH CDR2 comprises or consists of the amino acid sequence of a VH CDR2 of a VH set forth in Table 14, or the amino acid sequence of a VH CDR2 of a VH set forth in Table 14 comprising or consisting of 1, 2, or 3 amino acid variations (e.g., substitution, deletion, addition, etc.); the amino acid sequence of VH CDR3 comprises or consists of the amino acid sequence of a VH CDR3 of a VH set forth in Table 14, or the amino acid sequence of a VH CDR3 of a VH set forth in Table 14 comprising or consisting of 1, 2, or 3 amino acid variations (e.g., substitution, deletion, addition, etc.); the amino acid sequence of VL CDR1 comprises or consists of the amino acid sequence of a VL CDR1 of a VL set forth in Table 14, or the amino acid sequence of a VL CDR1 of a VL set forth in Table 14 comprising or consisting of 1, 2, or 3 amino acid variations (e.g., substitution, deletion, addition, etc.); the amino acid sequence of VL CDR2 comprises or consists of the amino acid sequence of a VL CDR2 of a VL set forth in Table 14, or the amino acid sequence of a VL CDR2 of a VL set forth in Table 14 comprising or consisting of 1, 2, or 3 amino acid variations (e.g., substitution, deletion, addition, etc.); and the amino acid sequence of VL CDR3 comprises or consists of the amino acid sequence of a VL CDR3 of a VL set forth in Table 14, or the amino acid sequence of a VL CDR3 of a VL set forth in Table 14 comprising or consisting of 1, 2, or 3 amino acid variations (e.g., substitution, deletion, addition, etc.).

In some embodiments, the amino acid sequence of VH CDR1 comprises or consists of the amino acid sequence of a VH CDR1 set forth in Table 14, or the amino acid sequence of a VH CDR1 set forth in Table 14 comprising or consisting of 1, 2, or 3 amino acid variations (e.g., substitution, deletion, addition, etc.); the amino acid sequence of VH CDR2 comprises or consists of the amino acid sequence of a VH CDR2 set forth in Table 14, or the amino acid sequence of a VH CDR2 set forth in Table 14 comprising or consisting of 1, 2, or 3 amino acid variations (e.g., substitution, deletion, addition, etc.); the amino acid sequence of VH CDR3 comprises or consists of the amino acid sequence of a VH CDR3 set forth in Table 14, or the amino acid sequence of a VH CDR3 set forth in Table 14 comprising or consisting of 1, 2, or 3 amino acid variations (e.g., substitution, deletion, addition, etc.); the amino acid sequence of VL CDR1 comprises or consists of the amino acid sequence of a VL CDR1 set forth in Table 14, or the amino acid sequence CDR1 of a VL set forth in Table 14 comprising or consisting of 1, 2, or 3 amino acid variations (e.g., substitution, deletion, addition, etc.); the amino acid sequence of VL CDR2 comprises or consists of the amino acid sequence of a VL CDR2 set forth in Table 14, or the amino acid sequence of a VL CDR2 set forth in Table 14 comprising or consisting of 1, 2, or 3 amino acid variations (e.g., substitution, deletion, addition, etc.); and the amino acid sequence of VL CDR3 comprises or consists of the amino acid sequence of a VL CDR3 set forth in Table 14, or the amino acid sequence of a VL CDR3 set forth in Table 14 comprising or consisting of 1, 2, or 3 amino acid variations (e.g., substitution, deletion, addition, etc.).

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 14. In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 14. In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 14; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 14.

### (b) Ig Constant Regions

In some embodiments, the antibody (or heterologous polypeptide (e.g., operably connected to a protein (e.g., hTF) that specifically binds TFR (e.g., described herein) comprises an IgG CH2 region and an IgG CH3 region. In some embodiments, the antibody (or heterologous polypeptide) comprises a partial IgG hinge region, IgG CH2 region, and IgG CH3 region. In some embodiments, the antibody (or heterologous polypeptide) comprises of an IgG hinge region, IgG CH2 region, and IgG CH3 region. In some embodiments, the antibody (or heterologous polypeptide) comprises an IgG1 CH2 region and an IgG1 CH3 region. In some embodiments, the antibody (or heterologous polypeptide) comprises a partial IgG1 hinge region, IgG1 CH2 region, and IgG1 CH3 region. In some embodiments, the antibody (or heterologous polypeptide) comprises an IgG1 hinge region, IgG1 CH2 region, and IgG1 CH3 region. In some embodiments, the antibody (or heterologous polypeptide) comprises an IgG4 CH2 region and an IgG4 CH3 region. In some embodiments, the antibody (or heterologous polypeptide) comprises a partial IgG4 hinge region, IgG4 CH2 region, and IgG4 CH3 region. In some embodiments, the antibody (or heterologous polypeptide) comprises an IgG4 hinge region, IgG4 CH2 region, and IgG4 CH3 region.

In some embodiments, the antibody (or heterologous polypeptide) comprises an Ig Fc region. In some embodiments, the Ig Fc region comprises or consists of at least a portion of a hinge region, a CH2 region, and a CH3 region. In some embodiments, the Ig Fc region comprises or consists of a hinge region, a CH2 region, and a CH3 region. In some embodiments, the Ig Fc region comprises or consists of at least a portion of an IgG hinge region, an IgG CH2 region, and an IgG CH3 region. In some embodiments, the Ig Fc region comprises or consists of an IgG hinge region, an IgG CH2 region, and an IgG CH3 region. In some embodiments, the Ig Fc region comprises or consists of at least a portion of an IgG1 hinge region, an IgG1 CH2 region, and an IgG1 CH3 region. In some embodiments, the Ig Fc region comprises or consists of an IgG1 hinge region, an IgG1 CH2 region, and an IgG1 CH3 region. In some embodiments, the Ig Fc region comprises or consists of at least a portion of an IgG4 hinge region, an IgG4 CH2 region, and an IgG4 CH3 region. In some embodiments, the Ig Fc region comprises or consists of an IgG4 hinge region, an IgG4 CH2 region, and an IgG4 CH3 region.

In some embodiments, the antibody (or heterologous polypeptide) comprises a first Ig Fc region and a second Ig Fc region. In some embodiments, the first and/or second Ig Fc region comprises or consists of at least a portion of a hinge region, a CH2 region, and a CH3 region. In some embodiments, the first and/or second Ig Fc region comprises or consists of a hinge region, a CH2 region, and a CH3 region. In some embodiments, the first and/or second Ig Fc region comprises or consists of at least a portion of an IgG hinge region, an IgG CH2 region, and an IgG CH3 region. In some embodiments, the first and/or second Ig Fc region comprises or consists of an IgG hinge region, an IgG CH2 region, and an IgG CH3 region. In some embodiments, the first and/or second Ig Fc region comprises or consists of at least a portion of an IgG1 hinge region, an IgG1 CH2 region, and an IgG1 CH3 region. In some embodiments, the first and/or second Ig Fc region comprises or consists of an IgG1 hinge region, an IgG1 CH2 region, and an IgG1 CH3 region. In some embodiments, the first and/or second Ig Fc region comprises or consists of at least a portion of an IgG4 hinge region, an IgG4 CH2 region, and an IgG4 CH3 region. In some embodiments, the first and/or second Ig Fc region comprises or consists of an IgG4 hinge region, an IgG4 CH2 region, and an IgG4 CH3 region.

In some embodiments, the antibody (or heterologous polypeptide) comprises one or more hIg heavy chain constant regions (e.g., a CH2 region, a CH3 region, a hinge region, an Fc region). In some embodiments, the hIg is a human IgG (hIgG). In some embodiments, the hIgG is hIgG1, IgG2, IgG3, or IgG4. In some embodiments, the hIgG is IgG1 or IgG4. In some embodiments, the hIgG is hIgG1. In some embodiments, the hIgG is hIgG4.

In some embodiments, the antibody (or heterologous polypeptide) comprises a hIgG CH2 region and a hIgG CH3 region. In some embodiments, the antibody (or heterologous polypeptide) comprises a partial hIgG hinge region, hIgG CH2 region, and hIgG CH3 region. In some embodiments, the antibody (or heterologous polypeptide) comprises a hIgG hinge region, hIgG CH2 region, and hIgG CH3 region. In some embodiments, the antibody (or heterologous polypeptide) comprises a hIgG1 CH2 region and a hIgG1 CH3 region. In some embodiments, the antibody (or heterologous polypeptide) comprises a partial hIgG1 hinge region, hIgG1 CH2 region, and hIgG1 CH3 region. In some embodiments, the antibody (or heterologous polypeptide) comprises a hIgG1 hinge region, hIgG1 CH2 region, and hIgG1 CH3 region. In some embodiments, the antibody (or heterologous polypeptide) comprises a hIgG4 CH2 region and a hIgG4 CH3 region. In some embodiments, the antibody (or heterologous polypeptide) comprises a partial hIgG4 hinge region, hIgG4 CH2 region, and hIgG4 CH3 region. In some embodiments, the antibody (or heterologous polypeptide) comprises a hIgG4 hinge region, hIgG4 CH2 region, and hIgG4 CH3 region.

In some embodiments, the antibody (or heterologous polypeptide) comprises a hIg Fc region. In some embodiments, the hIg Fc region comprises or consists of at least a portion of a hinge region, a CH2 region, and a CH3 region. In some embodiments, the hIg Fc region comprises or consists of a hinge region, a CH2 region, and a CH3 region. In some embodiments, the hIg Fc region comprises or consists of at least a portion of a hIgG hinge region, a hIgG CH2 region, and a hIgG CH3 region. In some embodiments, the hIg Fc region comprises or consists of a hIgG hinge region, a hIgG CH2 region, and a hIgG CH3 region. In some embodiments, the hIg Fc region comprises or consists of at least a portion of a hIgG1 hinge region, a hIgG1 CH2 region, and a hIgG1 CH3 region. In some embodiments, the hIg Fc region comprises or consists of a hIgG1 hinge region, a hIgG1 CH2 region, and a hIgG1 CH3 region. In some embodiments, the hIg Fc region comprises or consists of at least a portion of a hIgG4 hinge region, a hIgG4 CH2 region, and a hIgG4 CH3 region. In some embodiments, the hIg Fc region comprises or consists of a hIgG4 hinge region, a hIgG4 CH2 region, and a hIgG4 CH3 region.

In some embodiments, the antibody (or heterologous polypeptide) comprises a first hIg Fc region and a second hIg Fc region. In some embodiments, the first and/or second hIg Fc region comprises or consists of at least a portion of a hinge region, a CH2 region, and a CH3 region. In some embodiments, the first and/or second hIg Fc region comprises or consists of a hinge region, a CH2 region, and a CH3 region. In some embodiments, the first and/or second hIg Fc region comprises or consists of at least a portion of a hIgG hinge region, a hIgG CH2 region, and a hIgG CH3 region. In some embodiments, the first and/or second hIg Fc region comprises or consists of a hIgG hinge region, a hIgG CH2 region, and a hIgG CH3 region. In some embodiments, the first and/or second hIg Fc region comprises or consists of at least a portion of a hIgG1 hinge region, a hIgG1 CH2 region, and a hIgG1 CH3 region. In some embodiments, the first and/or second hIg Fc region comprises or consists of a hIgG1 hinge region, a hIgG1 CH2 region, and a hIgG1 CH3 region. In some embodiments, the first and/or second hIg Fc region comprises or consists of at least a portion of a hIgG4 hinge region, a hIgG4 CH2 region, and a hIgG4 CH3 region. In some embodiments, the first and/or second hIg Fc region comprises or consists of a hIgG4 hinge region, a hIgG4 CH2 region, and a hIgG4 CH3 region.

In some embodiments, the antibody (or heterologous polypeptide) comprises one or more Ig (e.g., hIg) light chain constant region (e.g., a hIg light chain kappa constant region (κCL) or a hIg light chain lambda constant region (λCL).

The amino acid sequence of exemplary reference hIgG1 and hIgG4 heavy chain and light chain constant regions, which can be incorporated in one or more of the embodiments described herein (e.g., anti-TFR (e.g., hTFR (e.g., hTFR1)) antibodies and heterologous polypeptides), is provided in Table 15.

### Table 15. The Amino Acid Sequence of Exemplary hIg Heavy Chain and Light Chain Constant Regions and Components Thereof.

| | | SEQ |
|---|---|---|
| Description | Amino Acid Sequence | ID NO |
| hIgG1 CH1 Region | ASTKGPSVFPLAPSSKSTSGGTAALGCLVKDYFPEPVTVSWNS | 3461 |
| | GALTSGVHTFPAVLQSSGLYSLSSVVTVPSSSLGTQTYICNVN | |
| | HKPSNTKVDKKV | |
| hIgG1 Hinge Region | EPKSCDKTHTCP | 3462 |
| hIgG1 CH2 Region | PCPAPELLGGPSVFLFPPKPKDTLMISRTPEVTCVVVDVSHED | 3463 |
| | PEVKFNWYVDGVEVHNAKTKPREEQYNSTYRVVSVLTVLHQDW | |
| | LNGKEYKCKVSNKALPAPIEKTISKAK | |
| hIgG1 CH3 Region | GQPREPQVYTLPPSRDELTKNQVSLTCLVKGFYPSDIAVEWES | 3464 |
| With C-terminal Lysine | NGQPENNYKTTPPVLDSDGSFFLYSKLTVDKSRWQQGNVFSCS | |
| | VMHEALHNHYTQKSLSLSPGK | |
| hIgG1 CH3 Region | GQPREPQVYTLPPSRDELTKNQVSLTCLVKGFYPSDIAVEWES | 3465 |
| Without C-terminal | NGQPENNYKTTPPVLDSDGSFFLYSKLTVDKSRWQQGNVFSCS | |
| Lysine | VMHEALHNHYTQKSLSLSPG | |
| hIgG1 CH2 Region + | PCPAPELLGGPSVFLFPPKPKDTLMISRTPEVTCVVVDVSHED | 3466 |
| CH3 Region | PEVKFNWYVDGVEVHNAKTKPREEQYNSTYRVVSVLIVLHQDW | |
| With C-terminal Lysine | LNGKEYKCKVSNKALPAPIEKTISKAKGQPREPQVYTLPPSRD | |
| | ELTKNQVSLTCLVKGFYPSDIAVEWESNGQPENNYKTTPPVLD | |
| | SDGSFFLYSKLTVDKSRWQQGNVFSCSVMHEALHNHYTQKSLS | |
| | LSPGK | |
| hIgG1 CH2 Region + | PCPAPELLGGPSVFLFPPKPKDTLMISRTPEVTCVVVDVSHED | 3467 |
| CH3 Region | PEVKFNWYVDGVEVHNAKTKPREEQYNSTYRVVSVLIVLHQDW | |
| Without C-terminal | LNGKEYKCKVSNKALPAPIEKTISKAKGQPREPQVYTLPPSRD | |
| Lysine | ELTKNQVSLTCLVKGFYPSDIAVEWESNGQPENNYKITPPVLD | |
| | SDGSFFLYSKLTVDKSRWQQGNVFSCSVMHEALHNHYTQKSLS | |
| | LSPG | |
| hIgG1 Partial Hinge | TCPPCPAPELLGGPSVFLFPPKPKDTLMISRTPEVTCVVVDVS | 3468 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| With C-terminal Lysine | SRDELTKNQVSLTCLVKGFYPSDIAVEWESNGQPENNYKITPP | |
| | VLDSDGSFFLYSKLTVDKSRWQQGNVFSCSVMHEALHNHYTQK | |
| | SLSLSPGK | |
| hIgG1 Partial Hinge | TCPPCPAPELLGGPSVFLFPPKPKDTLMISRTPEVTCVVVDVS | 3469 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Without C-terminal | SRDELTKNQVSLTCLVKGFYPSDIAVEWESNGQPENNYKITPP | |
| Lysine | VLDSDGSFFLYSKLTVDKSRWQQGNVFSCSVMHEALHNHYTQK | |
| | SLSLSPG | |
| hIgG1 Hinge Region + | EPKSCDKTHTCPPCPAPELLGGPSVFLFPPKPKDTLMISRTPE | 3470 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| With C-terminal Lysine | EPQVYTLPPSRDELTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPGK | |
| hIgG1 Hinge Region + | EPKSCDKTHTCPPCPAPELLGGPSVFLFPPKPKDILMISRIPE | 3471 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Without C-terminal | EPQVYTLPPSRDELTKNQVSLTCLVKGFYPSDIAVEWESNGQP | |
| Lysine | ENNYKTTPPVLDSDGSFFLYSKLTVDKSRWQQGNVFSCSVMHE | |
| | ALHNHYTQKSLSLSPG | |
| hIgG1 CH1 + Hinge | ASTKGPSVFPLAPSSKSTSGGTAALGCLVKDYFPEPVTVSWNS | 3472 |
| Region + CH2 Region + | GALISGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| With C-terminal Lysine | PKDTLMISRTPEVTCVVVDVSHEDPEVKFNWYVDGVEVHNAKT | |
| | KPREEQYNSTYRVVSVLIVLHQDWLNGKEYKCKVSNKALPAPI | |
| | EKTISKAKGQPREPQVYTLPPSRDELTKNQVSLTCLVKGFYPS | |
| | DIAVEWESNGQPENNYKTTPPVLDSDGSFFLYSKLTVDKSRWQ | |
| | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 3473 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Without C-terminal | PKDTLMISRTPEVTCVVVDVSHEDPEVKFNWYVDGVEVHNAKT | |
| Lysine | KPREEQYNSTYRVVSVLTVLHQDWLNGKEYKCKVSNKALPAPI | |
| | EKTISKAKGQPREPQVYTLPPSRDELTKNQVSLTCLVKGFYPS | |
| | DIAVEWESNGQPENNYKTTPPVLDSDGSFFLYSKLTVDKSRWQ | |
| | QGNVFSCSVMHEALHNHYTQKSLSLSPG | |
| hIgG4 CH1 Region | ASTKGPSVFPLAPCSRSTSESTAALGCLVKDYFPEPVTVSWNS | 3474 |
| | GALTSGVHTFPAVLQSSGLYSLSSVVTVPSSSLGTKTYTCNVD | |
| | HKPSNTKVDKRV | |
| hIgG4 Hinge Region | ESKYGPPCPSCP | 3475 |
| hIgG4 CH2 Region | APEFLGGPSVFLFPPKPKDTLMISRTPEVTCVVVDVSQEDPEV | 3476 |
| | QFNWYVDGVEVHNAKTKPREEQFNSTYRVVSVLTVLHQDWLNG | |
| | KEYKCKVSNKGLPSSIEKTISKAK | |
| hIgG4 CH3 Region | GQPREPQVYTLPPSQEEMTKNQVSLTCLVKGFYPSDIAVEWES | 3477 |
| With C-terminal Lysine | NGQPENNYKITPPVLDSDGSFFLYSRLTVDKSRWQEGNVFSCS | |
| | VMHEALHNHYTQKSLSLSLGK | |
| hIgG4 CH3 Region | GQPREPQVYTLPPSQEEMTKNQVSLTCLVKGFYPSDIAVEWES | 3478 |
| Without C-terminal | NGQPENNYKTTPPVLDSDGSFFLYSRLTVDKSRWQEGNVFSCS | |
| Lysine | VMHEALHNHYTQKSLSLSLG | |
| hIgG4 CH2 Region + | APEFLGGPSVFLFPPKPKDTLMISRTPEVTCVVVDVSQEDPEV | 3479 |
| CH3 Region | QFNWYVDGVEVHNAKTKPREEQFNSTYRVVSVLTVLHQDWLNG | |
| With C-terminal Lysine | KEYKCKVSNKGLPSSIEKTISKAKGQPREPQVYTLPPSQEEMT | |
| | KNQVSLTCLVKGFYPSDIAVEWESNGQPENNYKTTPPVLDSDG | |
| | SFFLYSRLTVDKSRWQEGNVFSCSVMHEALHNHYTQKSLSLSL | |
| | GK | |
| hIgG4 CH2 Region + | APEFLGGPSVFLFPPKPKDTLMISRTPEVTCVVVDVSQEDPEV | 3480 |
| CH3 Region | QFNWYVDGVEVHNAKTKPREEQFNSTYRVVSVLIVLHQDWLNG | |
| Without C-terminal | KEYKCKVSNKGLPSSIEKTISKAKGQPREPQVYTLPPSQEEMT | |
| Lysine | KNQVSLTCLVKGFYPSDIAVEWESNGQPENNYKTTPPVLDSDG | |
| | SFFLYSRLTVDKSRWQEGNVFSCSVMHEALHNHYTQKSLSLSL | |
| | G | |
| hIgG4 Partial Hinge | PCPSCPAPEFLGGPSVFLFPPKPKDTLMISRTPEVTCVVVDVS | 3481 |
| Region + CH2 Region + | QEDPEVQFNWYVDGVEVHNAKTKPREEQFNSTYRVVSVLTVLH | |
| CH3 Region | QDWLNGKEYKCKVSNKGLPSSIEKTISKAKGQPREPQVYTLPP | |
| With C-terminal Lysine | SQEEMTKNQVSLTCLVKGFYPSDIAVEWESNGQPENNYKITPP | |
| | VLDSDGSFFLYSRLTVDKSRWQEGNVFSCSVMHEALHNHYTQK | |
| | SLSLSLGK | |
| hIgG4 Partial Hinge | PCPSCPAPEFLGGPSVFLFPPKPKDTLMISRTPEVTCVVVDVS | 3482 |
| Region + CH2 Region + | QEDPEVQFNWYVDGVEVHNAKTKPREEQFNSTYRVVSVLTVLH | |
| CH3 Region | QDWLNGKEYKCKVSNKGLPSSIEKTISKAKGQPREPQVYTLPP | |
| Without C-terminal | SQEEMTKNQVSLTCLVKGFYPSDIAVEWESNGQPENNYKITPP | |
| Lysine | VLDSDGSFFLYSRLTVDKSRWQEGNVFSCSVMHEALHNHYTQK | |
| | SLSLSLG | |
| hIgG4 Hinge Region + | ESKYGPPCPSCPAPEFLGGPSVFLFPPKPKDTLMISRTPEVTC | 3483 |
| CH2 Region + CH3 | VVVDVSQEDPEVQFNWYVDGVEVHNAKTKPREEQFNSTYRVVS | |
| Region | VLTVLHQDWLNGKEYKCKVSNKGLPSSIEKTISKAKGQPREPQ | |
| With C-terminal Lysine | VYTLPPSQEEMTKNQVSLTCLVKGFYPSDIAVEWESNGQPENN | |
| | YKTTPPVLDSDGSFFLYSRLTVDKSRWQEGNVFSCSVMHEALH | |
| | NHYTQKSLSLSLGK | |
| hIgG4 Hinge Region + | ESKYGPPCPSCPAPEFLGGPSVFLFPPKPKDTLMISRTPEVTC | 3484 |
| CH2 Region + CH3 | VVVDVSQEDPEVQFNWYVDGVEVHNAKIKPREEQFNSTYRVVS | |
| Region | VLTVLHQDWLNGKEYKCKVSNKGLPSSIEKTISKAKGQPREPQ | |
| Without C-terminal | VYTLPPSQEEMTKNQVSLTCLVKGFYPSDIAVEWESNGQPENN | |
| Lysine | YKTTPPVLDSDGSFFLYSRLTVDKSRWQEGNVFSCSVMHEALH | |
| | NHYTQKSLSLSLG | |
| hIgG4 CH1 + Hinge | ASTKGPSVFPLAPCSRSTSESTAALGCLVKDYFPEPVTVSWNS | 3485 |
| Region + CH2 Region + | GALTSGVHTFPAVLQSSGLYSLSSVVTVPSSSLGTKTYTCNVD | |
| CH3 Region | HKPSNTKVDKRVESKYGPPCPSCPAPEFLGGPSVFLFPPKPKD | |
| With C-terminal Lysine | TLMISRTPEVTCVVVDVSQEDPEVQFNWYVDGVEVHNAKTKPR | |
| | EEQFNSTYRVVSVLIVLHQDWLNGKEYKCKVSNKGLPSSIEKT | |
| | ISKAKGQPREPQVYTLPPSQEEMTKNQVSLTCLVKGFYPSDIA | |
| | VEWESNGQPENNYKTTPPVLDSDGSFFLYSRLTVDKSRWQEGN | |
| | VFSCSVMHEALHNHYTQKSLSLSLGK | |
| hIgG4 CH1 + Hinge | ASTKGPSVFPLAPCSRSTSESTAALGCLVKDYFPEPVTVSWNS | 3486 |
| Region + CH2 Region + | GALTSGVHTFPAVLQSSGLYSLSSVVTVPSSSLGTKTYTCNVD | |
| CH3 Region | HKPSNTKVDKRVESKYGPPCPSCPAPEFLGGPSVFLFPPKPKD | |
| Without C-terminal | TLMISRTPEVTCVVVDVSQEDPEVQFNWYVDGVEVHNAKTKPR | |
| Lysine | EEQFNSTYRVVSVLIVLHQDWLNGKEYKCKVSNKGLPSSIEKT | |
| | ISKAKGQPREPQVYTLPPSQEEMTKNQVSLTCLVKGFYPSDIA | |
| | VEWESNGQPENNYKTTPPVLDSDGSFFLYSRLTVDKSRWQEGN | |
| | VFSCSVMHEALHNHYTQKSLSLSLG | |
| Ig light chain kappa | RTVAAPSVFIFPPSDEQLKSGTASVVCLLNNFYPREAKVQWKV | 3487 |
| constant region (κCL) | DNALQSGNSQESVTEQDSKDSTYSLSSTLILSKADYEKHKVYA | |
| | CEVTHQGLSSPVTKSFNRGEC | |
| Ig light chain kappa | GQPKANPTVTLFPPSSEELQANKATLVCLISDFYPGAVTVAWK | 3489 |
| constant region (λCL) | ADGSPVKAGVETTKPSKQSNNKYAASSYLSLTPEQWKSHRSYS | |
| | CQVTHEGSTVEKTVAPTECS | |

In some embodiments, the antibody (or heterologous polypeptide) comprises one or more hIg constant region, wherein the amino acid sequence of the one or more hIg constant region comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 15. In some embodiments, the amino acid sequence of the one or more hIg constant region comprises or consists of an amino acid sequence set forth in Table 15.

In some embodiments, the amino acid sequence of the one or more hIg constant region comprises or consists of an amino acid sequence set forth in Table 15, and further comprises 1 or more but less than 15% (less than 12%, less than 10%, less than 8%), amino acid variations (e.g., amino acid substitutions, deletions, or additions). In some embodiments, the amino acid sequence of the one or more hIg constant region comprises or consists of an amino acid sequence set forth in Table 15, comprising or consisting of at least about 1, 2, 3, 4, 5, 6, 7, 8, 9, 10, or more amino acid variations (e.g., amino acid substitutions, deletions, or additions). In some embodiments, the amino acid sequence of the one or more hIg constant region comprises or consists of an amino acid sequence set forth in Table 15, comprising or consisting of about 1, 2, 3, 4, 5, 6, 7, 8, 9, 10, or more amino acid variations (e.g., amino acid substitutions, deletions, or additions). In some embodiments, the amino acid sequence of the one or more hIg constant regions comprises or consists of an amino acid sequence set forth in Table 15, comprising or consisting of about no more than 1, 2, 3, 4, 5, 6, 7, 8, 9, 10, or more amino acid variations (e.g., amino acid substitutions, deletions, or additions).

In some embodiments, the amino acid sequence of the one or more hIg constant regions comprises or consists of an amino acid sequence set forth in Table 15, and further comprises 1 or more but less than 15% (less than 12%, less than 10%, less than 8%), amino acid substitutions. In some embodiments, the amino acid sequence of the one or more hIg constant regions comprises or consists of an amino acid sequence set forth in Table 15, comprising or consisting of at least about 1, 2, 3, 4, 5, 6, 7, 8, 9, 10, or more amino acid substitutions. In some embodiments, the amino acid sequence of the one or more hIg constant regions comprises or consists of an amino acid sequence set forth in Table 15, comprising or consisting of about 1, 2, 3, 4, 5, 6, 7, 8, 9, 10, or more amino acid substitutions. In some embodiments, the amino acid sequence of the one or more hIg constant regions comprises or consists of an amino acid sequence set forth in Table 15, comprising or consisting of about no more than 1, 2, 3, 4, 5, 6, 7, 8, 9, 10, or more amino acid substitutions.

In some embodiments, the amino acid sequence of the one or more hIg constant regions comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3461-3489. In some embodiments, the amino acid sequence of the one or more hIg constant regions comprises or consists of the amino acid sequence of any one of SEQ ID NOS: 3461-3489.

In some embodiments, the amino acid sequence of the one or more hIg constant regions comprises or consists of the amino acid sequence of any one of SEQ ID NOS: 3461-3489, and further comprises 1 or more but less than 15% (less than 12%, less than 10%, less than 8%), amino acid variations (e.g., amino acid substitutions, deletions, or additions). In some embodiments, the amino acid sequence of the one or more hIg constant regions comprises or consists of the amino acid sequence of any one of SEQ ID NOS: 3461-3489, comprising or consisting at least about 1, 2, 3, 4, 5, 6, 7, 8, 9, 10, or more amino acid variations (e.g., amino acid substitutions, deletions, or additions). In some embodiments, the amino acid sequence of the one or more hIg constant regions comprises or consists of the amino acid sequence of any one of SEQ ID NOS: 3461-3489, comprising or consisting about 1, 2, 3, 4, 5, 6, 7, 8, 9, or 10 amino acid variations (e.g., amino acid substitutions, deletions, or additions). In some embodiments, the amino acid sequence of the one or more hIg constant regions comprises or consists of the amino acid sequence of any one of SEQ ID NOS: 3461-3489, comprising or consisting of no more than about 1, 2, 3, 4, 5, 6, 7, 8, 9, or 10 amino acid variations (e.g., amino acid substitutions, deletions, or additions).

In some embodiments, the amino acid sequence of the one or more hIg constant regions comprises or consists of the amino acid sequence of any one of SEQ ID NOS: 3461-3489, and further comprises 1 or more but less than 15% (less than 12%, less than 10%, less than 8%), amino acid substitutions. In some embodiments, the amino acid sequence of the one or more hIg constant regions comprises or consists of the amino acid sequence of any one of SEQ ID NOS: 3461-3489, comprising or consisting at least about 1, 2, 3, 4, 5, 6, 7, 8, 9, 10, or more amino acid substitutions. In some embodiments, the amino acid sequence of the one or more hIg constant regions comprises or consists of the amino acid sequence of any one of SEQ ID NOS: 3461-3489, comprising or consisting about 1, 2, 3, 4, 5, 6, 7, 8, 9, or 10 amino acid substitutions. In some embodiments, the amino acid sequence of the one or more hIg constant regions comprises or consists of the amino acid sequence of any one of SEQ ID NOS: 3461-3489, comprising or consisting of no more than about 1, 2, 3, 4, 5, 6, 7, 8, 9, or 10 amino acid substitutions.

### Ig Effector Function

As described herein, in some embodiments, the antibody (or heterologous polypeptide) comprises an Fc region (e.g., described herein). In some embodiments, the Fc region of an antibody (or heterologous polypeptide) described herein exhibits a decrease in one or more Fc effector function relative to a reference (e.g., wild type) Fc region. Exemplary Fc effector functions include, but are not limited to, antibody dependent cellular cytotoxicity (ADCC), antibody dependent cellular phagocytosis (ADCP), complement dependent cytotoxicity (CDC), and binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

Standard in vitro and/or in vivo assays known in the art can be conducted to evaluate Fc effector function, including, any one or more of ADCC, CDC, ADCP, Fc receptor (e.g., Fcγ receptor) binding affinity, and C1q binding affinity.

For example, ADCC activity can be assessed utilizing standard (radioactive and non-radioactive) methods known in the art (see, e.g., WO2006/082515, WO2012/130831), the entire contents of each of which is incorporated by reference herein for all purposes). For example, ADCC activity can be assessed using a chromium-5 (⁵¹Cr) assay. Briefly, ⁵¹Cr is pre-loaded into target cells, NK cells are added to the culture, and radioactivity in the cell culture supernatant is assessed (indicative of lysis of the target cells by the NK cells). Similar non-radioactive assays can also be utilized that employ a similar method, but the target cells are pre-loaded with fluorescent dyes, such as calcein-AM, CFSE, BCECF, or lanthanide fluorophore (Europium). See, e.g., Parekh, Bhavin S et al. “Development and validation of an antibody-dependent cell-mediated cytotoxicity-reporter gene assay.” mAbs vol. 4, 3 (2012): 310-8. Doi:10.4161/mabs.19873, the entire contents of which is incorporated by reference herein for all purposes. Exemplary commercially available non-radioactive assays include, for example, ACTI™ non-radioactive cytotoxicity assay for flow cytometry (Cell Technology, Inc. Mountain View, Calif.; and CytoTox 96® non-radioactive cytotoxicity assay (Promega, Madison, Wis.). Additional non-limiting examples of in vitro assays that can be used to assess ADCC activity of a fusion protein described herein include those described in U.S. Pat. Nos. 5,500,362; 5,821,337; Hellstrom, I., et al., Proc. Nat'l Acad. Sci. USA 83 (1986) 7059-7063; Hellstrom, I., et al., Proc. Nat'l Acad. Sci. USA 82 (1985) 1499-1502; and Bruggemann, M., et al., J. Exp. Med. 166 (1987) 1351-1361, the entire contents of each of which is incorporated by reference herein. Alternatively, or additionally, ADCC activity of a fusion protein described herein may be assessed in vivo, e.g., in an animal model such as that disclosed in Clynes, et al., Proc. Nat'l Acad. Sci. USA 95 (1998) 652-656, the entire contents of which is incorporated by reference herein for all purposes.

C1q binding assays can be utilized to assess the ability of a an antibody (or heterologous polypeptide) described herein to bind C1q (or bind with less affinity than a reference fusion protein) and hence lack (or have decreased) CDC activity. The binding of an antibody (or heterologous polypeptide) described herein to C1q can be determined by a variety of in vitro assays (e.g., biochemical or immunological based assays) known in the art for determining Fc-C1q interactions, including e.g., equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4^{th} Ed., Lippincott-Raven, Philadelphia (1999), the entire contents of which is incorporated by reference herein. For example, see, e.g., C1q and C3c binding ELISAs described in WO2006/029879 and WO2005/100402, the entire contents of each of which is incorporated by reference herein for all purposes. Additional CDC activity assays include those described in e.g., Gazzano-Santoro, et al., J. Immunol. Methods 202 (1996) 163; Cragg, M. S., et al., Blood 101 (2003) 1045-1052; and Cragg, M. S., and Glennie, M. J., Blood 103 (2004) 2738-2743), the entire contents of each of which is incorporated by reference herein for all purposes.

ADCP activity can be measured by in vitro or in vivo methods known in the art and also commercially available assays (see, e.g., van de Donk N W, Moreau P, Plesner T, et al. “Clinical efficacy and management of monoclonal antibodies targeting CD38 and SLAMF7 in multiple myeloma,” Blood, 127(6):681-695 (2016), the entire contents of each of which is incorporated by reference herein for all purposes). For example, a primary cell based ADCP assay can be used in which fresh human peripheral blood mononuclear cells (PBMCs) are isolated, monocytes isolated and differentiated in culture to macrophages using standard procedures. The macrophages are fluorescently labeled added to cultures containing fluorescently labeled target cells. Phagocytosis events can be analyzed using FACS screening and/or microscopy. A modified reporter version of the above described assay can also be used that employs an engineered cell line that stably expresses FcγRIIa (CD32a) as the effector cell line (e.g., an engineered T cell line, e.g., THP-1), removing the requirement for primary cells. Exemplary ADCP assays are described in e.g., Ackerman, M. E. et al. A robust, high-throughput assay to determine the phagocytic activity of clinical antibody samples. J. Immunol. Methods 366, 8-19 (2011); and Mcandrew, E. G. et al. Determining the phagocytic activity of clinical antibody samples. J. Vis. Exp. 3588 (2011). Doi:10.3791/3588; the entire contents of each of which is incorporated by reference herein.

Binding of an antibody (or heterologous polypeptide) described herein to an Fc receptor can be determined by a variety of in vitro assays (e.g., biochemical or immunological based assays) known in the art for determining Fc-Fc receptor interactions, i.e., specific binding of an Fc region to an Fc receptor. Common assays include equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4” Ed., Lippincott-Raven, Philadelphia (1999), the entire contents of which is incorporated by reference herein for all purposes.

In some embodiments, the Fc region of an antibody (or heterologous polypeptide) described herein is varied (e.g., comprises one or more amino acid variation (e.g., one or more amino acid substitution, deletion, addition, etc.)) (referred to herein as a “varied Fc region”), relative to the amino acid sequence of a reference Fc region (e.g., a wild type Fc region, e.g., Table 15 herein (e.g., SEQ ID NOS: 178, 180, 191, or 195). In some embodiments, the one or more amino acid variation (e.g., the one or more amino acid substitution, deletion, addition, etc.)) decreases or abolishes one or more Fc effector function, relative to a reference Fc that does not comprise the variation (e.g., the one or more variation (e.g., the one or more amino acid substitution, deletion, addition, etc.)).

In some embodiments, the antibody (or heterologous polypeptide) comprising a varied Fc region exhibits no detectable or decreased ADCC compared to a reference antibody (or heterologous polypeptide) that does not comprise the Fc region variation (e.g., the one or more amino acid variation (e.g., one or more amino acid substitution, deletion, or addition)). In some embodiments, the antibody (or heterologous polypeptide) comprising a varied Fc region exhibits no detectable or decreased CDC compared to a reference antibody (or heterologous polypeptide) that does not comprise the Fc region variation (e.g., the one or more amino acid variation (e.g., one or more amino acid substitution, deletion, or addition)). In some embodiments, the antibody (or heterologous polypeptide) comprising a modified Fc region exhibits no detectable or decreased ADCP compared to a reference antibody (or heterologous polypeptide) that does not comprise the Fc region variation (e.g., the one or more variation (e.g., one or more amino acid substitution, deletion, or addition)). In some embodiments, the antibody (or heterologous polypeptide) comprising a varied Fc region exhibits decreased or no detectable specific binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))) compared to a reference antibody (or heterologous polypeptide) that does not comprise the Fc region variation (e.g., the one or more variation (e.g., one or more amino acid substitution, deletion, or addition)). In some embodiments, the antibody (or heterologous polypeptide) comprising a varied Fc region exhibits decreased or no detectable specific binding affinity to FcγRI, FcγIIa, and/or FcγIIIa compared to an antibody (or heterologous polypeptide) that does not comprise the Fc region variation (e.g., the one or more variation (e.g., one or more amino acid substitution, deletion, or addition)). In some embodiments, the antibody (or heterologous polypeptide) comprising a varied Fc exhibits decreased or no detectable specific binding affinity to FcγRI compared to a reference antibody (or heterologous polypeptide) that does not comprise the Fc variation (e.g., the one or more variation (e.g., one or more amino acid substitution, deletion, or addition)). In some embodiments, the antibody (or heterologous polypeptide) comprising a varied Fc exhibits decreased or no detectable specific binding affinity to FcγIIa compared to a reference antibody (or heterologous polypeptide) that does not comprise the Fc region variation (e.g., the one or more variation (e.g., one or more amino acid substitution, deletion, or addition)). In some embodiments, the antibody (or heterologous polypeptide) comprising a varied Fc region exhibits decreased or no detectable specific binding affinity to FcγIIIa compared to an antibody (or heterologous polypeptide) that does not comprise the Fc region variation (e.g., the one or more variation (e.g., one or more amino acid substitution, deletion, or addition)). In some embodiments, the antibody (or heterologous polypeptide) comprising a varied Fc region exhibits decreased or no detectable specific binding affinity to C1q compared to a reference antibody (or heterologous polypeptide) that does not comprise the Fc region variation (e.g., the one or more variation (e.g., one or more amino acid substitution, deletion, or addition)).

In some embodiments, the antibody (or heterologous polypeptide) comprising an Fc region exhibits no detectable ADCC. In some embodiments, the antibody (or heterologous polypeptide) comprising an Fc region exhibits no detectable CDC. In some embodiments, the antibody (or heterologous polypeptide) comprising an Fc region exhibits no detectable ADCP. In some embodiments, the antibody (or heterologous polypeptide) comprising an Fc region exhibits no detectable specific binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))). In some embodiments, the antibody (or heterologous polypeptide) comprising an Fc region exhibits no detectable specific binding affinity to FcγRI, FcγIIa, and/or. In some embodiments, the antibody (or heterologous polypeptide) comprising an Fc exhibits no detectable specific binding affinity to FcγRI. In some embodiments, the antibody (or heterologous polypeptide) comprising an Fc exhibits no detectable specific binding affinity to FcγIIa. In some embodiments, the antibody (or heterologous polypeptide) comprising an Fc region exhibits no detectable specific binding affinity to FcγIIIa. In some embodiments, the antibody (or heterologous polypeptide) comprising an Fc region exhibits no detectable specific binding affinity to C1q.

Amino acid substitutions that decrease or abolish one or more Fc effector function are known in the art. See for example, Saunders Kevin, “Conceptual Approaches to Modulating Antibody Effector Functions and Circulation Half-Life,” Frontiers in Immunology, v10 (Jun. 7, 2019) DOI=10.3389/fimmu.2019.01296, the full contents of which is incorporated by reference herein for all purposes, see more particularly for example, e.g., Table 8 of Saunders.

In some embodiments, the varied Fc comprises a hIgG1 Fc region comprising one or more amino acid variations (e.g., one or more amino acid substitutions). In some embodiments, the hIgG1 Fc region comprises an amino acid substitution at amino acid positions L234, L235, and/or P329, EU numbering according to Kabat. In some embodiments, the hIgG1 Fc region comprises the following amino acid substitutions L234A and/or L235A, EU numbering according to Kabat. In some embodiments, the hIgG1 Fc region comprises the following amino acid substitutions L234A, L235A, and P329G, EU numbering according to Kabat. In some embodiments, the hIgG1 Fc region comprises the following amino acid substitutions L234A, L235A, and P329A, EU numbering according to Kabat.

In some embodiments, the varied Fc region comprises a hIg4 Fc region comprising one or more amino acid variations (e.g., one or more amino acid substitutions). In some embodiments, the hIgG4 Fc region comprises an amino acid substitution at amino acid positions D228, F234, and/or L235, EU numbering according to Kabat. In some embodiments, the hIgG4 Fc region comprises the following amino acid substitutions S228P, F234A, and/or L235A, EU numbering according to Kabat. In some embodiments, the hgG4 Fc region comprises the following amino acid substitutions S228P, F234A, and/or L235E, EU numbering according to Kabat. In some embodiments, the hIgG4 Fc comprises the following amino acid substitutions S228P and/or L235E, EU numbering according to Kabat.

The amino acid sequence of exemplary varied Fc regions that are known in the art to exhibit a decrease in one more effector function is provided in Table 16.

TABLE 16
The Amino Acid Sequence of Exemplary Varied Fc Regions.

| | | SEQ |
|---|---|---|
| Description | Amino Acid Sequence | ID NO |
| hIgG1 CH2 Region + | PCPAPEAAGGPSVFLFPPKPKDTLMISRTPEVTCVVVDVSHED | 3490 |
| CH3 Region | PEVKFNWYVDGVEVHNAKTKPREEQYNSTYRVVSVLIVLHQDW | |
| L234A/L235A | LNGKEYKCKVSNKALPAPIEKTISKAKGQPREPQVYTLPPSRD | |
| With C-terminal Lysine | ELTKNQVSLTCLVKGFYPSDIAVEWESNGQPENNYKTTPPVLD | |
| | SDGSFFLYSKLTVDKSRWQQGNVFSCSVMHEALHNHYTQKSLS | |
| | LSPGK | |
| hIgG1 CH2 Region + | PCPAPEAAGGPSVFLFPPKPKDTLMISRTPEVTCVVVDVSHED | 3491 |
| CH3 Region | PEVKFNWYVDGVEVHNAKIKPREEQYNSTYRVVSVLTVLHQDW | |
| L234A/L235A | LNGKEYKCKVSNKALPAPIEKTISKAKGQPREPQVYTLPPSRD | |
| Without C-terminal | ELTKNQVSLTCLVKGFYPSDIAVEWESNGQPENNYKTTPPVLD | |
| Lysine | SDGSFFLYSKLTVDKSRWQQGNVFSCSVMHEALHNHYTQKSLS | |
| | LSPG | |
| hIgG1 CH2 Region + | PCPAPEAAGGPSVFLFPPKPKDTLMISRTPEVTCVVVDVSHED | 3492 |
| CH3 Region | PEVKFNWYVDGVEVHNAKTKPREEQYNSTYRVVSVLIVLHQDW | |
| L234A/L235A/P329A | LNGKEYKCKVSNKALAAPIEKTISKAKGQPREPQVYTLPPSRD | |
| With C-terminal Lysine | ELTKNQVSLTCLVKGFYPSDIAVEWESNGQPENNYKTTPPVLD | |
| | SDGSFFLYSKLTVDKSRWQQGNVFSCSVMHEALHNHYTQKSLS | |
| | LSPGK | |
| hIgG1 CH2 Region + | PCPAPEAAGGPSVFLFPPKPKDTLMISRTPEVTCVVVDVSHED | 3493 |
| CH3 Region | PEVKFNWYVDGVEVHNAKTKPREEQYNSTYRVVSVLIVLHQDW | |
| L234A/L235A/P329A | LNGKEYKCKVSNKALAAPIEKTISKAKGQPREPQVYTLPPSRD | |
| Without C-terminal | ELTKNQVSLTCLVKGFYPSDIAVEWESNGQPENNYKTTPPVLD | |
| Lysine | SDGSFFLYSKLTVDKSRWQQGNVFSCSVMHEALHNHYTQKSLS | |
| | LSPG | |
| hIgG1 CH2 Region + | PCPAPEAAGGPSVFLFPPKPKDTLMISRTPEVTCVVVDVSHED | 3494 |
| CH3 Region | PEVKFNWYVDGVEVHNAKTKPREEQYNSTYRVVSVLIVLHQDW | |
| L234A/L235A/P329G | LNGKEYKCKVSNKALGAPIEKTISKAKGQPREPQVYTLPPSRD | |
| With C-terminal Lysine | ELTKNQVSLTCLVKGFYPSDIAVEWESNGQPENNYKITPPVLD | |
| | SDGSFFLYSKLTVDKSRWQQGNVFSCSVMHEALHNHYTQKSLS | |
| | LSPGK | |
| hIgG1 CH2 Region + | PCPAPEAAGGPSVFLFPPKPKDTLMISRTPEVTCVVVDVSHED | 3495 |
| CH3 Region | PEVKFNWYVDGVEVHNAKTKPREEQYNSTYRVVSVLTVLHQDW | |
| L234A/L235A/P329G | LNGKEYKCKVSNKALGAPIEKTISKAKGQPREPQVYTLPPSRD | |
| Without C-terminal | ELTKNQVSLTCLVKGFYPSDIAVEWESNGQPENNYKTTPPVLD | |
| Lysine | SDGSFFLYSKLTVDKSRWQQGNVFSCSVMHEALHNHYTQKSLS | |
| | LSPG | |
| hIgG1 Partial Hinge | TCPPCPAPEAAGGPSVFLFPPKPKDTLMISRTPEVTCVVVDVS | 3496 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| L234A/L235A | SRDELTKNQVSLTCLVKGFYPSDIAVEWESNGQPENNYKTTPP | |
| With C-terminal Lysine | VLDSDGSFFLYSKLTVDKSRWQQGNVFSCSVMHEALHNHYTQK | |
| | SLSLSPGK | |
| hIgG1 Partial Hinge | TCPPCPAPEAAGGPSVFLFPPKPKDTLMISRTPEVTCVVVDVS | 3497 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| L234A/L235A | SRDELTKNQVSLTCLVKGFYPSDIAVEWESNGQPENNYKTTPP | |
| Without C-terminal | VLDSDGSFFLYSKLTVDKSRWQQGNVFSCSVMHEALHNHYTQK | |
| Lysine | SLSLSPG | |
| hIgG1 Partial Hinge | TCPPCPAPEAAGGPSVFLFPPKPKDTLMISRTPEVTCVVVDVS | 3498 |
| Region + CH2 Region + | HEDPEVKFNWYVDGVEVHNAKTKPREEQYNSTYRVVSVLTVLH | |
| CH3 Region | QDWLNGKEYKCKVSNKALAAPIEKTISKAKGQPREPQVYTLPP | |
| L234A/L235A/P329A | SRDELTKNQVSLTCLVKGFYPSDIAVEWESNGQPENNYKTTPP | |
| With C-terminal Lysine | VLDSDGSFFLYSKLTVDKSRWQQGNVFSCSVMHEALHNHYTQK | |
| | SLSLSPGK | |
| hIgG1 Partial Hinge | TCPPCPAPEAAGGPSVFLFPPKPKDTLMISRTPEVTCVVVDVS | 3499 |
| Region + CH2 Region + | HEDPEVKFNWYVDGVEVHNAKTKPREEQYNSTYRVVSVLTVLH | |
| CH3 Region | QDWLNGKEYKCKVSNKALAAPIEKTISKAKGQPREPQVYTLPP | |
| L234A/L235A/P329A | SRDELTKNQVSLTCLVKGFYPSDIAVEWESNGQPENNYKTTPP | |
| Without C-terminal | VLDSDGSFFLYSKLTVDKSRWQQGNVFSCSVMHEALHNHYTQK | |
| Lysine | SLSLSPG | |
| hIgG1 Partial Hinge | TCPPCPAPEAAGGPSVFLFPPKPKDTLMISRTPEVTCVVVDVS | 3500 |
| Region + CH2 Region + | HEDPEVKFNWYVDGVEVHNAKTKPREEQYNSTYRVVSVLTVLH | |
| CH3 Region | QDWLNGKEYKCKVSNKALGAPIEKTISKAKGQPREPQVYTLPP | |
| L234A/L235A/P329G | SRDELTKNQVSLTCLVKGFYPSDIAVEWESNGQPENNYKTTPP | |
| With C-terminal Lysine | VLDSDGSFFLYSKLTVDKSRWQQGNVFSCSVMHEALHNHYTQK | |
| | SLSLSPGK | |
| hIgG1 Partial Hinge | TCPPCPAPEAAGGPSVFLFPPKPKDTLMISRTPEVTCVVVDVS | 3501 |
| Region + CH2 Region + | HEDPEVKFNWYVDGVEVHNAKTKPREEQYNSTYRVVSVLTVLH | |
| CH3 Region | QDWLNGKEYKCKVSNKALGAPIEKTISKAKGQPREPQVYTLPP | |
| L234A/L235A/P329G | SRDELTKNQVSLTCLVKGFYPSDIAVEWESNGQPENNYKTTPP | |
| Without C-terminal | VLDSDGSFFLYSKLTVDKSRWQQGNVFSCSVMHEALHNHYTQK | |
| Lysine | SLSLSPG | |
| hIgG1 Hinge Region + | EPKSCDKTHTCPPCPAPEAAGGPSVFLFPPKPKDTLMISRTPE | 3502 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| L234A/L235A | EPQVYTLPPSRDELTKNQVSLTCLVKGFYPSDIAVEWESNGQP | |
| With C-terminal Lysine | ENNYKTTPPVLDSDGSFFLYSKLTVDKSRWQQGNVFSCSVMHE | |
| | ALHNHYTQKSLSLSPGK | |
| hIgG1 Hinge Region + | EPKSCDKTHTCPPCPAPEAAGGPSVFLFPPKPKDTLMISRTPE | 3503 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| L234A/L235A | EPQVYTLPPSRDELTKNQVSLTCLVKGFYPSDIAVEWESNGQP | |
| Without C-terminal | ENNYKTTPPVLDSDGSFFLYSKLTVDKSRWQQGNVFSCSVMHE | |
| Lysine | ALHNHYTQKSLSLSPG | |
| hIgG1 Hinge Region + | EPKSCDKTHTCPPCPAPEAAGGPSVFLFPPKPKDTLMISRTPE | 3504 |
| CH2 Region + CH3 | VTCVVVDVSHEDPEVKFNWYVDGVEVHNAKTKPREEQYNSTYR | |
| Region | VVSVLIVLHQDWLNGKEYKCKVSNKALPAPIEKTISKAKGQPR | |
| L234A/L235A/P329A | EPQVYTLPPSRDELTKNQVSLTCLVKGFYPSDIAVEWESNGQP | |
| With C-terminal Lysine | ENNYKTTPPVLDSDGSFFLYSKLTVDKSRWQQGNVFSCSVMHE | |
| | ALHNHYTQKSLSLSPGK | |
| hIgG1 Hinge Region + | EPKSCDKTHTCPPCPAPEAAGGPSVFLFPPKPKDTLMISRTPE | 3505 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| L234A/L235A/P329A | EPQVYTLPPSRDELTKNQVSLTCLVKGFYPSDIAVEWESNGQP | |
| Without C-terminal | ENNYKTTPPVLDSDGSFFLYSKLTVDKSRWQQGNVFSCSVMHE | |
| Lysine | ALHNHYTQKSLSLSPG | |
| hIgG1 Hinge Region + | EPKSCDKTHTCPPCPAPEAAGGPSVFLFPPKPKDTLMISRTPE | 3506 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| L234A/L235A/P329G | EPQVYTLPPSRDELTKNQVSLTCLVKGFYPSDIAVEWESNGQP | |
| With C-terminal Lysine | ENNYKTTPPVLDSDGSFFLYSKLTVDKSRWQQGNVFSCSVMHE | |
| | ALHNHYTQKSLSLSPGK | |
| hIgG1 Hinge Region + | EPKSCDKTHTCPPCPAPEAAGGPSVFLFPPKPKDTLMISRTPE | 3507 |
| CH2 Region + CH3 | VTCVVVDVSHEDPEVKFNWYVDGVEVHNAKTKPREEQYNSTYR | |
| Region | VVSVLIVLHQDWLNGKEYKCKVSNKALPAPIEKTISKAKGQPR | |
| L234A/L235A/P329G | EPQVYTLPPSRDELTKNQVSLTCLVKGFYPSDIAVEWESNGQP | |
| Without C-terminal | ENNYKTTPPVLDSDGSFFLYSKLTVDKSRWQQGNVFSCSVMHE | |
| Lysine | ALHNHYTQKSLSLSPG | |
| hIgG4 CH2 Region + | APEAAGGPSVFLFPPKPKDTLMISRTPEVTCVVVDVSQEDPEV | 3508 |
| CH3 Region | QFNWYVDGVEVHNAKTKPREEQFNSTYRVVSVLTVLHQDWLNG | |
| S228P/F234A/L235A | KEYKCKVSNKGLPSSIEKTISKAKGQPREPQVYTLPPSQEEMT | |
| With C-terminal Lysine | KNQVSLTCLVKGFYPSDIAVEWESNGQPENNYKTTPPVLDSDG | |
| | SFFLYSRLTVDKSRWQEGNVFSCSVMHEALHNHYTQKSLSLSL | |
| | GK | |
| hIgG4 CH2 Region + | APEAAGGPSVFLFPPKPKDTLMISRTPEVTCVVVDVSQEDPEV | 3509 |
| CH3 Region | QFNWYVDGVEVHNAKTKPREEQFNSTYRVVSVLTVLHQDWLNG | |
| S228P/F234A/L235A | KEYKCKVSNKGLPSSIEKTISKAKGQPREPQVYTLPPSQEEMT | |
| Without C-terminal | KNQVSLTCLVKGFYPSDIAVEWESNGQPENNYKTTPPVLDSDG | |
| Lysine | SFFLYSRLTVDKSRWQEGNVFSCSVMHEALHNHYTQKSLSLSL | |
| | G | |
| hIgG4 Partial Hinge | PCPSCPAPEAAGGPSVFLFPPKPKDTLMISRTPEVTCVVVDVS | 3510 |
| Region + CH2 Region + | QEDPEVQFNWYVDGVEVHNAKTKPREEQFNSTYRVVSVLTVLH | |
| CH3 Region | QDWLNGKEYKCKVSNKGLPSSIEKTISKAKGQPREPQVYTLPP | |
| S228P/F234A/L235A | SQEEMTKNQVSLTCLVKGFYPSDIAVEWESNGQPENNYKITPP | |
| With C-terminal Lysine | VLDSDGSFFLYSRLTVDKSRWQEGNVFSCSVMHEALHNHYTQK | |
| | SLSLSLGK | |
| hIgG4 Partial Hinge | PCPPCPAPEAAGGPSVFLFPPKPKDTLMISRTPEVTCVVVDVS | 3511 |
| Region + CH2 Region + | QEDPEVQFNWYVDGVEVHNAKTKPREEQFNSTYRVVSVLTVLH | |
| CH3 Region | QDWLNGKEYKCKVSNKGLPSSIEKTISKAKGQPREPQVYTLPP | |
| S228P/F234A/L235A | SQEEMTKNQVSLTCLVKGFYPSDIAVEWESNGQPENNYKTTPP | |
| Without C-terminal | VLDSDGSFFLYSRLTVDKSRWQEGNVFSCSVMHEALHNHYTQK | |
| Lysine | SLSLSLG | |
| hIgG4 Hinge Region + | ESKYGPPCPPCPAPEAAGGPSVFLFPPKPKDTLMISRTPEVTC | 3512 |
| CH2 Region + CH3 | VVVDVSQEDPEVQFNWYVDGVEVHNAKTKPREEQFNSTYRVVS | |
| Region | VLTVLHQDWLNGKEYKCKVSNKGLPSSIEKTISKAKGQPREPQ | |
| S228P/F234A/L235A | VYTLPPSQEEMTKNQVSLTCLVKGFYPSDIAVEWESNGQPENN | |
| With C-terminal Lysine | YKTTPPVLDSDGSFFLYSRLTVDKSRWQEGNVFSCSVMHEALH | |
| | NHYTQKSLSLSLGK | |
| hIgG4 Hinge Region + | ESKYGPPCPPCPAPEAAGGPSVFLFPPKPKDTLMISRTPEVTC | 3513 |
| CH2 Region + CH3 | VVVDVSQEDPEVQFNWYVDGVEVHNAKTKPREEQFNSTYRVVS | |
| Region | VLTVLHQDWLNGKEYKCKVSNKGLPSSIEKTISKAKGQPREPQ | |
| S228P/F234A/L235A | VYTLPPSQEEMTKNQVSLTCLVKGFYPSDIAVEWESNGQPENN | |
| Without C-terminal | YKITPPVLDSDGSFFLYSRLTVDKSRWQEGNVFSCSVMHEALH | |
| Lysine | NHYTQKSLSLSLG | |
| hIgG4 Hinge Region + | AESKYGPPCPPCPAPEAAGGPSVFLFPPKPKDTLMISRTPEVT | 3514 |
| CH2 Region + CH3 | CVVVDVSQEDPEVQFNWYVDGVEVHNAKTKPREEQFNSTYRVV | |
| Region | SVLTVLHQDWLNGKEYKCKVSNKGLPSSIEKTISKAKGQPREP | |
| (Modified) | QVYTLPPSQEEMTKNQVSLTCLVKGFYPSDIAVEWESNGQPEN | |
| S228P/F234A/L235A | NYKITPPVLDSDGSFFLYSRLTVDKSRWQEGNVFSCSVMHEAL | |
| With C-terminal Lysine | HNHYTQKSLSLSLGK | |
| hIgG4 Hinge Region + | AESKYGPPCPPCPAPEAAGGPSVFLFPPKPKDTLMISRTPEVT | 3515 |
| CH2 Region + CH3 | CVVVDVSQEDPEVQFNWYVDGVEVHNAKTKPREEQFNSTYRVV | |
| Region | SVLTVLHQDWLNGKEYKCKVSNKGLPSSIEKTISKAKGQPREP | |
| (Modified) | QVYTLPPSQEEMTKNQVSLTCLVKGFYPSDIAVEWESNGQPEN | |
| S228P/F234A/L235A | NYKTTPPVLDSDGSFFLYSRLTVDKSRWQEGNVFSCSVMHEAL | |
| Without C-terminal | HNHYTQKSLSLSLG | |
| Lysine | | |

In some embodiments, the variant hIg Fc fusion protein or polypeptide comprises a hIg Fc region comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 16.

In some embodiments, the amino acid sequence of the variant hIg Fc fusion protein or polypeptide comprises a hIg Fc region that comprises or consists of the amino acid sequence of a polypeptide set forth in Table 16, and further comprises 1 or more but less than 15% (less than 12%, less than 10%, less than 8%), amino acid variations (e.g., amino acid substitutions, deletions, or additions). In some embodiments, the amino acid sequence of the variant hIg Fc fusion protein or polypeptide comprises a hIg Fc region that comprises or consists of the amino acid sequence of a polypeptide set forth in Table 16, and further comprises or consists of at least about 1, 2, 3, 4, 5, 6, 7, 8, 9, or 10 amino acid variations (e.g., substitutions, additions, deletions, etc.). In some embodiments, the amino acid sequence of the variant hIg Fc fusion protein or polypeptide comprises a hIg Fc region that comprises or consists of the amino acid sequence of a polypeptide set forth in Table 16, and further comprises or consists of about 1, 2, 3, 4, 5, 6, 7, 8, 9, or 10 amino acid variations (e.g., substitutions, additions, deletions, etc.). In some embodiments, the amino acid sequence of the variant hIg Fc fusion protein or polypeptide comprises a hIg Fc region that comprises or consists of the amino acid sequence of a polypeptide set forth in Table 16, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the variant hIg Fc fusion protein or polypeptide comprises a hIg Fc region that comprises or consists of the amino acid sequence of a polypeptide set forth in Table 16, and further comprises 1 or more but less than 15% (less than 12%, less than 10%, less than 8%), amino acid substitutions. In some embodiments, the amino acid sequence of the variant hIg Fc fusion protein or polypeptide comprises a hIg Fc region that comprises or consists of the amino acid sequence of a polypeptide set forth in Table 16, and further comprises or consists of at least about 1, 2, 3, 4, 5, 6, 7, 8, 9, or 10 amino acid substitutions. In some embodiments, the amino acid sequence of the variant hIg Fc fusion protein or polypeptide comprises a hIg Fc region that comprises or consists of the amino acid sequence of a polypeptide set forth in Table 16, and further comprises or consists of about 1, 2, 3, 4, 5, 6, 7, 8, 9, or 10 amino acid substitutions. In some embodiments, the amino acid sequence of the variant hIg Fc fusion protein or polypeptide comprises a hIg Fc region that comprises or consists of the amino acid sequence of a polypeptide set forth in Table 16, and further comprises or consists of no more than about 1, 2, 3, 4, 5, 6, 7, 8, 9, or 10 amino acid substitutions.

In some embodiments, the amino acid sequence of the variant hIg Fc fusion protein or polypeptide comprises a hIg Fc region that comprises or consists of an amino acid sequence at least 85%, 86%, 87%, 88%, 89%, 90%, 91%, 92%, 93%, 94%, 95%, 96%, 97%, 98%, or 100% identical to the amino acid sequence of any one of SEQ ID NOS: 3490-3515.

In some embodiments, the amino acid sequence of the variant hIg Fc fusion protein or polypeptide comprises a hIg Fc region that comprises or consists of the amino acid sequence of any one of SEQ ID NOS: 3490-3515, and further comprises 1 or more but less than 15% (less than 12%, less than 10%, less than 8%), amino acid variations (e.g., amino acid substitutions, deletions, or additions). In some embodiments, the amino acid sequence of the variant hIg Fc fusion protein or polypeptide comprises a hIg Fc region that comprises or consists of the amino acid sequence of any one of SEQ ID NOS: 3490-3515, and further comprises or consists of at least about 1, 2, 3, 4, 5, 6, 7, 8, 9, or 10 amino acid variations (e.g., substitutions, additions, deletions, etc.). In some embodiments, the amino acid sequence of the variant hIg Fc fusion protein or polypeptide comprises a hIg Fc region that comprises or consists of the amino acid sequence of any one of SEQ ID NOS: 3490-3515, and further comprises or consists of about 1, 2, 3, 4, 5, 6, 7, 8, 9, or 10 amino acid variations (e.g., substitutions, additions, deletions, etc.). In some embodiments, the amino acid sequence of the variant hIg Fc fusion protein or polypeptide comprises a hIg Fc region that comprises or consists of the amino acid sequence of any one of SEQ ID NOS: 3490-3515, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the variant hIg Fc fusion protein or polypeptide comprises a hIg Fc region that comprises or consists of the amino acid sequence of any one of SEQ ID NOS: 3490-3515, and further comprises 1 or more but less than 15% (less than 12%, less than 10%, less than 8%), amino acid substitutions. In some embodiments, the amino acid sequence of the variant hIg Fc fusion protein or polypeptide comprises a hIg Fc region that comprises or consists of the amino acid sequence of any one of SEQ ID NOS: 3490-3515, and further comprises or consists of at least about 1, 2, 3, 4, 5, 6, 7, 8, 9, or 10 amino acid substitutions. In some embodiments, the amino acid sequence of the variant hIg Fc fusion protein or polypeptide comprises a hIg Fc region that comprises or consists of the amino acid sequence of any one of SEQ ID NOS: 3490-3515, and further comprises or consists of about 1, 2, 3, 4, 5, 6, 7, 8, 9, or 10 amino acid substitutions. In some embodiments, the amino acid sequence of the variant hIg Fc fusion protein or polypeptide comprises a hIg Fc region that comprises or consists of the amino acid sequence of any one of SEQ ID NOS: 3490-3515, and further comprises or consists of no more than about 1, 2, 3, 4, 5, 6, 7, 8, 9, or 10 amino acid substitutions.

In some embodiments, the amino acid sequence of the hIgG1 Fc region comprises an alanine at position L234 and/or an alanine at position L235, EU numbering according to Kabat. In some embodiments, the amino acid sequence of the hIgG1 Fc region comprises an alanine at position L234 and an alanine at position L235, EU numbering according to Kabat. In some embodiments, the amino acid sequence of the hIgG1 Fc region comprises of an alanine, glycine, or serine at position P329, EU numbering according to Kabat.

In some embodiments, the amino acid sequence of the hIgG1 Fc region comprises an alanine at position L234; an alanine at position L235; and an alanine, glycine, or serine at position P329 EU numbering according to Kabat. In some embodiments, the amino acid sequence of the hIgG1 Fc region comprises of an alanine at position L234; an alanine at position L235; and an alanine at position P329 EU numbering according to Kabat. In some embodiments, the amino acid sequence of the hIgG1 Fc region comprises an alanine at position L234; an alanine at position L235; and a glycine amino acid at position P329 EU numbering according to Kabat. In some embodiments, the amino acid sequence of the hIgG1 Fc region comprises an alanine at position L234; an alanine at position L235; and a serine amino acid at position P329 EU numbering according to Kabat.

In some embodiments, the amino acid sequence of the hIgG1 Fc region comprises an alanine at position L234, an alanine at position L235, and/or an alanine, glycine, or serine at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 16. In some embodiments, the amino acid sequence of the hIgG1 Fc region comprises an alanine at position L234 and/or an alanine at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 16. In some embodiments, the amino acid sequence of the hIgG1 Fc region comprises an alanine at position L234 and an alanine at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 16. In some embodiments, the amino acid sequence of the hIgG1 Fc region comprises an alanine at position L234, an alanine at position L235, and an alanine, glycine, or serine at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 16. In some embodiments, the amino acid sequence of the hIgG1 Fc region comprises an alanine at position L234, an alanine at position L235, and an alanine at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 16. In some embodiments, the amino acid sequence of the hIgG1 Fc region comprises an alanine at position L234, an alanine at position L235, and a glycine at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 16. In some embodiments, the amino acid sequence of the hIgG1 Fc region comprises an alanine at position L234, an alanine at position L235, and a serine at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 16.

In some embodiments, the amino acid sequence of the hIgG1 Fc region comprises an alanine at position L234, an alanine at position L235, and/or an alanine, glycine, or serine amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG1 Fc region comprises an alanine at position L234 and/or an alanine at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG1 Fc region comprises an alanine at position L234 and an alanine at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG1 Fc region comprises an alanine at position L234, an alanine at position L235, and an alanine, glycine, or serine amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG1 Fc region comprises an alanine at position L234, an alanine at position L235, and an alanine at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG1 Fc region comprises an alanine at position L234, an alanine at position L235, and a glycine amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG1 Fc region comprises an alanine at position L234, an alanine at position L235, and a serine amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515.

In some embodiments, the amino acid sequence of the hIgG1 Fc region comprises an alanine at position L234, an alanine at position L235, and/or an alanine, glycine, or serine amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG1 Fc region comprises an alanine at position L234 and/or an alanine at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG1 Fc region comprises an alanine at position L234 and an alanine at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG1 Fc region comprises an alanine at position L234, an alanine at position L235, and an alanine, glycine, or serine amino acid at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG1 Fc region comprises an alanine at position L234, an alanine at position L235, and an alanine at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG1 Fc region comprises an alanine at position L234, an alanine at position L235, and a glycine at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG1 Fc region comprises an alanine at position L234, an alanine at position L235, and a serine at position P329, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515.

In some embodiments, the amino acid sequence of the hIgG4 Fc region comprises a phenylalanine at position L234 and/or an alanine at position L235, EU numbering according to Kabat. In some embodiments, the amino acid sequence of the hIgG4 Fc region comprises a phenylalanine at position L234 and an alanine at position L235, EU numbering according to Kabat. In some embodiments, the amino acid sequence of the hIgG4 Fc region comprises a proline at position S228, EU numbering according to Kabat.

In some embodiments, the amino acid sequence of the hIgG4 Fc region comprises a phenylalanine at position L234; an alanine at position L235; and a proline at position S228, EU numbering according to Kabat.

In some embodiments, the amino acid sequence of the hIgG4 Fc region comprises a phenylalanine at position L234, an alanine at position L235, and/or a proline at position S228, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 16. In some embodiments, the amino acid sequence of the hIgG4 Fc region comprises a phenylalanine at position L234 and/or an alanine at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 16. In some embodiments, the amino acid sequence of the hIgG4 Fc region comprises a phenylalanine at position L234, an alanine at position L235, and a proline at position S228, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 16.

In some embodiments, the amino acid sequence of the hIgG4 Fc region comprises a phenylalanine at position L234, an alanine at position L235, and/or a proline at position S228, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS:199-224. In some embodiments, the amino acid sequence of the hIgG4 Fc region comprises a phenylalanine at position L234 and/or an alanine at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG4 Fc region comprises a phenylalanine at position L234, an alanine at position L235, and a proline at position S228, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515.

In some embodiments, the amino acid sequence of the hIgG4 Fc region comprises a phenylalanine at position L234, an alanine at position L235, and/or a proline at position S228, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG4 Fc region comprises a phenylalanine at position L234 and/or an alanine at position L235, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515. In some embodiments, the amino acid sequence of the hIgG4 Fc region comprises a phenylalanine at position L234, an alanine at position L235, and a proline at position S228, EU numbering according to Kabat; and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 3490-3515.

### Promotion of Heterodimerization

As described herein, in some embodiments, the antibody (or heterologous polypeptide) comprises a first and second Fc region (e.g., described herein). In some embodiments, the first Ig Fc region and the second Ig Fc region each comprise one or more amino acid modifications relative to each other to promote heterodimerization. IgG derived heterodimeric formats can be generated by methods known in the art, e.g., by forced heavy chain heterodimerization. Forced heavy chain heterodimerization can be obtained using known methods in the art, e.g., knob-in-hole or strand exchange engineered domains (SEED), see, e.g., Ji-Hee et al., “Immunoglobulin Fc Heterodimer Platform Technology: From Design to Applications in Therapeutic Antibodies and Proteins” Frontiers in Immunology, v7 (article 394) (2016) DOI=10.3389/fimmu.2016.00394 (hereinafter “Ji-Hee 2016”), the entire contents of which is incorporated by reference herein for all purposes.

In some embodiments, an interface of the first and the second Ig Fc regions is varied, e.g., introduction of an amino acid substitution, to increase heterodimerization, e.g., relative to a non-modified interface, e.g., a naturally occurring interface. For example, dimerization of the first and second Ig Fc regions can be enhanced by providing an Ig Fc interface of a first and a second Fc region with one or more of: a paired protuberance-cavity (“knob-in-hole”), an electrostatic interaction, or a strand-exchange, such that a greater ratio of heteromultimer to homomultimer forms, e.g., relative to a non-modified interface.

Knob-in-Hole amino acid pairing modifications are known in the art, and described in e.g., U.S. Pat. Nos. 5,731,116; 7,476,724; Ji-Hee 2016; and Ridgway, J. “‘Knobs-into-holes’ engineering of antibody CH3 domains for heavy chain heterodimerization” et al. Prot. Engineering 9(7): 617-621 (1996), the full contents of each of which is incorporated by reference herein. Generally, Knob-in-Hole comprises 1) introducing one or more amino acid substitutions in the CH3 domain of one or both of the first and second subject Ig Fc regions to promote heterodimerization; and 2) combining the modified Ig Fc regions under conditions that promote heterodimerization. “Knobs” are typically created by substituting a small amino acid in a parental Ig Fc region with a larger amino acid (e.g., T366Y or T366W); “holes” are created by substituting a larger residue in a parental Ig Fc region with a smaller amino acid (e.g., Y407T, T366S, 11368A, or Y407V). Exemplary Knob-in-Hole mutations include S354C, T366W in the “knob” Ig Fc region and Y349C, T366S, L368A, Y407V in the “hole” Ig Fc region. Other exemplary Knob-in-Hole mutations, which can be incorporated into any one or more of the embodiments, are provided in Table 17, with additional exemplary optional stabilizing Ig Fc cysteine mutations.

TABLE 17
Exemplary Knob-in-hole and Stabilizing Cysteine Modifications.

| | | |
|---|---|---|
| Amino Acid Position | | |
| (EU numbering according to | Knob Ig Fc Amino Acid | Hole Ig Fc Amino Acid |
| Kabat) | Substitution | Substitution |

Knob-in Hole Amio Acid Substitutions

| | | |
|---|---|---|
| T366 | T366W | T366S |
| L368 | — | L368A |
| Y407 | — | Y407V |

Stabilizing Cysteine Amino Acid Substitutions

| | | |
|---|---|---|
| S354 | S354C | — |
| Y349 | — | Y349C |

The amino acid sequence of exemplary Fc regions that are known in the art to promote heterodimerization is provided in Table 18.

TABLE 18
The Amino Acid Sequence of Exemplary Pairs of
Varied Heterodimeric Fc Regions.

| | | SEQ |
|---|---|---|
| Description | Amino Acid Sequence | ID NO |
| hIgG1 CH2 Region + | PCPAPELLGGPSVFLFPPKPKDTLMISRTPEVTCVVVDVSHED | 3516 |
| CH3 Region | PEVKFNWYVDGVEVHNAKTKPREEQYNSTYRVVSVLIVLHQDW | |
| Knob T366W | LNGKEYKCKVSNKALPAPIEKTISKAKGQPREPQVYTLPPSRD | |
| With C-terminal Lysine | ELTKNQVSLWCLVKGFYPSDIAVEWESNGQPENNYKITPPVLD | |
| | SDGSFFLYSKLTVDKSRWQQGNVFSCSVMHEALHNHYTQKSLS | |
| | LSPGK | |
| hIgG1 CH2 Region + | PCPAPELLGGPSVFLFPPKPKDTLMISRTPEVTCVVVDVSHED | 3517 |
| CH3 Region | PEVKFNWYVDGVEVHNAKTKPREEQYNSTYRVVSVLIVLHQDW | |
| Knob T366W | LNGKEYKCKVSNKALPAPIEKTISKAKGQPREPQVYTLPPSRD | |
| Without C-terminal | ELTKNQVSLWCLVKGFYPSDIAVEWESNGQPENNYKTTPPVLD | |
| Lysine | SDGSFFLYSKLTVDKSRWQQGNVFSCSVMHEALHNHYTQKSLS | |
| | LSPG | |
| hIgG1 Partial Hinge | TCPPCPAPELLGGPSVFLFPPKPKDTLMISRTPEVTCVVVDVS | 3518 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Knob T366W | SRDELTKNQVSLWCLVKGFYPSDIAVEWESNGQPENNYKITPP | |
| With C-terminal Lysine | VLDSDGSFFLYSKLTVDKSRWQQGNVFSCSVMHEALHNHYTQK | |
| | SLSLSPGK | |
| hIgG1 Partial Hinge | TCPPCPAPELLGGPSVFLFPPKPKDTLMISRTPEVTCVVVDVS | 3519 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Knob T366W | SRDELTKNQVSLWCLVKGFYPSDIAVEWESNGQPENNYKTTPP | |
| Without C-terminal | VLDSDGSFFLYSKLTVDKSRWQQGNVFSCSVMHEALHNHYTQK | |
| Lysine | SLSLSPG | |
| hIgG1 Hinge Region + | EPKSCDKTHTCPPCPAPELLGGPSVFLFPPKPKDTLMISRTPE | 3520 |
| CH2 Region + CH3 | VTCVVVDVSHEDPEVKFNWYVDGVEVHNAKTKPREEQYNSTYR | |
| Region | VVSVLIVLHQDWLNGKEYKCKVSNKALPAPIEKTISKAKGQPR | |
| Knob T366W | EPQVYTLPPSRDELTKNQVSLWCLVKGFYPSDIAVEWESNGQP | |
| With C-terminal Lysine | ENNYKTTPPVLDSDGSFFLYSKLTVDKSRWQQGNVFSCSVMHE | |
| | ALHNHYTQKSLSLSPGK | |
| hIgG1 Hinge Region + | EPKSCDKTHTCPPCPAPELLGGPSVFLFPPKPKDTLMISRTPE | 3521 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Knob T366W | EPQVYTLPPSRDELTKNQVSLWCLVKGFYPSDIAVEWESNGQP | |
| Without C-terminal | ENNYKTTPPVLDSDGSFFLYSKLTVDKSRWQQGNVFSCSVMHE | |
| Lysine | ALHNHYTQKSLSLSPG | |
| hIgG1 CH1+ Hinge | ASTKGPSVFPLAPSSKSTSGGTAALGCLVKDYFPEPVTVSWNS | 3522 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Knob T366W | PKDTLMISRTPEVTCVVVDVSHEDPEVKFNWYVDGVEVHNAKT | |
| With C-terminal Lysine | KPREEQYNSTYRVVSVLTVLHQDWLNGKEYKCKVSNKALPAPI | |
| | EKTISKAKGQPREPQVYTLPPCRDELTKNQVSLWCLVKGFYPS | |
| | DIAVEWESNGQPENNYKTTPPVLDSDGSFFLYSKLTVDKSRWQ | |
| | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 3523 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Knob T366W | PKDTLMISRTPEVTCVVVDVSHEDPEVKFNWYVDGVEVHNAKT | |
| Without C-terminal | KPREEQYNSTYRVVSVLIVLHQDWLNGKEYKCKVSNKALPAPI | |
| Lysine | EKTISKAKGQPREPQVYTLPPSRDELTKNQVSLWCLVKGFYPS | |
| | DIAVEWESNGQPENNYKTTPPVLDSDGSFFLYSKLTVDKSRWQ | |
| | QGNVFSCSVMHEALHNHYTQKSLSLSPG | |
| hIgG1 CH2 Region + | PCPAPELLGGPSVFLFPPKPKDTLMISRTPEVTCVVVDVSHED | 3524 |
| CH3 Region | PEVKFNWYVDGVEVHNAKTKPREEQYNSTYRVVSVLIVLHQDW | |
| Knob T366W/S354C | LNGKEYKCKVSNKALPAPIEKTISKAKGQPREPQVYTLPPCRD | |
| With C-terminal Lysine | ELTKNQVSLWCLVKGFYPSDIAVEWESNGQPENNYKTTPPVLD | |
| | SDGSFFLYSKLTVDKSRWQQGNVFSCSVMHEALHNHYTQKSLS | |
| | LSPGK | |
| hIgG1 CH2 Region + | PCPAPELLGGPSVFLFPPKPKDTLMISRTPEVTCVVVDVSHED | 3525 |
| CH3 Region | PEVKFNWYVDGVEVHNAKTKPREEQYNSTYRVVSVLIVLHQDW | |
| Knob T366W/S354C | LNGKEYKCKVSNKALPAPIEKTISKAKGQPREPQVYTLPPCRD | |
| Without C-terminal | ELTKNQVSLWCLVKGFYPSDIAVEWESNGQPENNYKTTPPVLD | |
| Lysine | SDGSFFLYSKLTVDKSRWQQGNVFSCSVMHEALHNHYTQKSLS | |
| | LSPG | |
| hIgG1 Partial Hinge | TCPPCPAPELLGGPSVFLFPPKPKDTLMISRTPEVTCVVVDVS | 3526 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Knob T366W/S354C | CRDELTKNQVSLWCLVKGFYPSDIAVEWESNGQPENNYKITPP | |
| With C-terminal Lysine | VLDSDGSFFLYSKLTVDKSRWQQGNVFSCSVMHEALHNHYTQK | |
| | SLSLSPGK | |
| hIgG1 Partial Hinge | TCPPCPAPELLGGPSVFLFPPKPKDTLMISRTPEVTCVVVDVS | 3527 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Knob T366W/S354C | CRDELTKNQVSLWCLVKGFYPSDIAVEWESNGQPENNYKTTPP | |
| Without C-terminal | VLDSDGSFFLYSKLTVDKSRWQQGNVFSCSVMHEALHNHYTQK | |
| Lysine | SLSLSPG | |
| hIgG1 Hinge Region + | EPKSCDKTHTCPPCPAPELLGGPSVFLFPPKPKDTLMISRTPE | 3528 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Knob T366W/S354C | EPQVYTLPPCRDELTKNQVSLWCLVKGFYPSDIAVEWESNGQP | |
| With C-terminal Lysine | ENNYKTTPPVLDSDGSFFLYSKLTVDKSRWQQGNVFSCSVMHE | |
| | ALHNHYTQKSLSLSPGK | |
| hIgG1 Hinge Region + | EPKSCDKTHTCPPCPAPELLGGPSVFLFPPKPKDTLMISRTPE | 3529 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Knob T366W/S354C | EPQVYTLPPCRDELTKNQVSLWCLVKGFYPSDIAVEWESNGQP | |
| Without C-terminal | ENNYKTTPPVLDSDGSFFLYSKLTVDKSRWQQGNVFSCSVMHE | |
| Lysine | ALHNHYTQKSLSLSPG | |
| hIgG1 CH1 + Hinge | ASTKGPSVFPLAPSSKSTSGGTAALGCLVKDYFPEPVTVSWNS | 3530 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Knob T366W/S354C | PKDTLMISRTPEVTCVVVDVSHEDPEVKFNWYVDGVEVHNAKT | |
| With C-terminal Lysine | KPREEQYNSTYRVVSVLTVLHQDWLNGKEYKCKVSNKALPAPI | |
| | EKTISKAKGQPREPQVYTLPPCRDELTKNQVSLWCLVKGFYPS | |
| | DIAVEWESNGQPENNYKTTPPVLDSDGSFFLYSKLTVDKSRWQ | |
| | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 3531 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Knob T366W/S354C | PKDTLMISRTPEVTCVVVDVSHEDPEVKFNWYVDGVEVHNAKT | |
| Without C-terminal | KPREEQYNSTYRVVSVLTVLHQDWLNGKEYKCKVSNKALPAPI | |
| Lysine | EKTISKAKGQPREPQVYTLPPCRDELTKNQVSLWCLVKGFYPS | |
| | DIAVEWESNGQPENNYKTTPPVLDSDGSFFLYSKLTVDKSRWQ | |
| | QGNVFSCSVMHEALHNHYTQKSLSLSPG | |
| hIgG1 CH2 Region + | PCPAPELLGGPSVFLFPPKPKDTLMISRTPEVTCVVVDVSHED | 3532 |
| CH3 Region | PEVKFNWYVDGVEVHNAKTKPREEQYNSTYRVVSVLTVLHQDW | |
| Hole | LNGKEYKCKVSNKALPAPIEKTISKAKGQPREPQVYTLPPSRD | |
| T366S/L368A/Y407V | ELTKNQVSLSCAVKGFYPSDIAVEWESNGQPENNYKTTPPVLD | |
| With C-terminal Lysine | SDGSFFLVSKLTVDKSRWQQGNVFSCSVMHEALHNHYTQKSLS | |
| | LSPGK | |
| hIgG1 CH2 Region + | PCPAPELLGGPSVFLFPPKPKDTLMISRTPEVTCVVVDVSHED | 3533 |
| CH3 Region | PEVKFNWYVDGVEVHNAKTKPREEQYNSTYRVVSVLTVLHQDW | |
| Hole | LNGKEYKCKVSNKALPAPIEKTISKAKGQPREPQVYTLPPSRD | |
| T366S/L368A/Y407V | ELTKNQVSLSCAVKGFYPSDIAVEWESNGQPENNYKTTPPVLD | |
| Without C-terminal | SDGSFFLVSKLTVDKSRWQQGNVFSCSVMHEALHNHYTQKSLS | |
| Lysine | LSPG | |
| hIgG1 Partial Hinge | TCPPCPAPELLGGPSVFLFPPKPKDTLMISRTPEVTCVVVDVS | 3534 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Hole | SRDELTKNQVSLSCAVKGFYPSDIAVEWESNGQPENNYKTTPP | |
| T366S/L368A/Y407V | VLDSDGSFFLVSKLTVDKSRWQQGNVFSCSVMHEALHNHYTQK | |
| With C-terminal Lysine | SLSLSPGK | |
| hIgG1 Partial Hinge | TCPPCPAPELLGGPSVFLFPPKPKDTLMISRTPEVTCVVVDVS | 3535 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVYTLPP | |
| Hole | SRDELTKNQVSLSCAVKGFYPSDIAVEWESNGQPENNYKTTPP | |
| T366S/L368A/Y407V | VLDSDGSFFLVSKLTVDKSRWQQGNVFSCSVMHEALHNHYTQK | |
| Without C-terminal | SLSLSPG | |
| Lysine | | |
| hIgG1 Hinge Region + | EPKSCDKTHTCPPCPAPELLGGPSVFLFPPKPKDTLMISRTPE | 3536 |
| CH2 Region + CH3 | VTCVVVDVSHEDPEVKFNWYVDGVEVHNAKTKPREEQYNSTYR | |
| Region | VVSVLIVLHQDWLNGKEYKCKVSNKALPAPIEKTISKAKGQPR | |
| Hole | EPQVYTLPPSRDELTKNQVSLSCAVKGFYPSDIAVEWESNGQP | |
| T366S/L368A/Y407V | ENNYKTTPPVLDSDGSFFLVSKLTVDKSRWQQGNVFSCSVMHE | |
| With C-terminal Lysine | ALHNHYTQKSLSLSPGK | |
| hIgG1 Hinge Region + | EPKSCDKTHTCPPCPAPELLGGPSVFLFPPKPKDTLMISRTPE | 3537 |
| CH2 Region + CH3 | VTCVVVDVSHEDPEVKFNWYVDGVEVHNAKTKPREEQYNSTYR | |
| Region | VVSVLIVLHQDWLNGKEYKCKVSNKALPAPIEKTISKAKGQPR | |
| Hole | EPQVYTLPPSRDELTKNQVSLSCAVKGFYPSDIAVEWESNGQP | |
| T366S/L368A/Y407V | ENNYKTTPPVLDSDGSFFLVSKLTVDKSRWQQGNVFSCSVMHE | |
| Without C-terminal | ALHNHYTQKSLSLSPG | |
| Lysine | | |
| hIgG1 CH1+ Hinge | ASTKGPSVFPLAPSSKSTSGGTAALGCLVKDYFPEPVTVSWNS | 3538 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Hole | PKDTLMISRTPEVTCVVVDVSHEDPEVKFNWYVDGVEVHNAKT | |
| T366S/L368A/Y407V | KPREEQYNSTYRVVSVLTVLHQDWLNGKEYKCKVSNKALPAPI | |
| With C-terminal Lysine | EKTISKAKGQPREPQVYTLPPSRDELTKNQVSLSCAVKGFYPS | |
| | DIAVEWESNGQPENNYKTTPPVLDSDGSFFLVSKLTVDKSRWQ | |
| | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 3539 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Hole | PKDTLMISRTPEVTCVVVDVSHEDPEVKFNWYVDGVEVHNAKT | |
| T366S/L368A/Y407V | KPREEQYNSTYRVVSVLIVLHQDWLNGKEYKCKVSNKALPAPI | |
| Without C-terminal | EKTISKAKGQPREPQVYTLPPSRDELTKNQVSLSCAVKGFYPS | |
| Lysine | DIAVEWESNGQPENNYKITPPVLDSDGSFFLVSKLTVDKSRWQ | |
| | QGNVFSCSVMHEALHNHYTQKSLSLSPG | |
| hIgG1 CH2 Region + | PCPAPELLGGPSVFLFPPKPKDTLMISRTPEVTCVVVDVSHED | 3540 |
| CH3 Region | PEVKFNWYVDGVEVHNAKTKPREEQYNSTYRVVSVLIVLHQDW | |
| Hole | LNGKEYKCKVSNKALPAPIEKTISKAKGQPREPQVYTLPPSRD | |
| T366S/L368A/Y407V/Y349C | ELTKNQVSLSCAVKGFYPSDIAVEWESNGQPENNYKTTPPVLD | |
| With C-terminal Lysine | SDGSFFLVSKLTVDKSRWQQGNVFSCSVMHEALHNHYTQKSLS | |
| | LSPGK | |
| hIgG1 CH2 Region + | PCPAPELLGGPSVFLFPPKPKDTLMISRTPEVTCVVVDVSHED | 3541 |
| CH3 Region | PEVKFNWYVDGVEVHNAKTKPREEQYNSTYRVVSVLTVLHQDW | |
| Hole | LNGKEYKCKVSNKALPAPIEKTISKAKGQPREPQVYTLPPSRD | |
| T366S/L368A/Y407V/Y349C | ELTKNQVSLSCAVKGFYPSDIAVEWESNGQPENNYKTTPPVLD | |
| Without C-terminal | SDGSFFLVSKLTVDKSRWQQGNVFSCSVMHEALHNHYTQKSLS | |
| Lysine | LSPG | |
| hIgG1 Partial Hinge | TCPPCPAPELLGGPSVFLFPPKPKDTLMISRTPEVTCVVVDVS | 3542 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVCTLPP | |
| Hole | SRDELTKNQVSLSCAVKGFYPSDIAVEWESNGQPENNYKTTPP | |
| T366S/L368A/Y407V/Y349C | VLDSDGSFFLVSKLTVDKSRWQQGNVFSCSVMHEALHNHYTQK | |
| With C-terminal Lysine | SLSLSPGK | |
| hIgG1 Partial Hinge | TCPPCPAPELLGGPSVFLFPPKPKDTLMISRTPEVTCVVVDVS | 3543 |
| Region + CH2 Region + | HEDPEVKFNWYVDGVEVHNAKTKPREEQYNSTYRVVSVLTVLH | |
| CH3 Region | QDWLNGKEYKCKVSNKALPAPIEKTISKAKGQPREPQVCTLPP | |
| Hole | SRDELTKNQVSLSCAVKGFYPSDIAVEWESNGQPENNYKTTPP | |
| T366S/L368A/Y407V/Y348C | VLDSDGSFFLVSKLTVDKSRWQQGNVFSCSVMHEALHNHYTQK | |
| Without C-terminal | SLSLSPG | |
| Lysine | | |
| hIgG1 Hinge Region + | EPKSCDKTHTCPPCPAPELLGGPSVFLFPPKPKDTLMISRTPE | 3544 |
| CH2 Region + CH3 | VTCVVVDVSHEDPEVKFNWYVDGVEVHNAKTKPREEQYNSTYR | |
| Region | VVSVLIVLHQDWLNGKEYKCKVSNKALPAPIEKTISKAKGQPR | |
| Hole | EPQVCTLPPSRDELTKNQVSLSCAVKGFYPSDIAVEWESNGQP | |
| T366S/L368A/Y407V/Y349C | ENNYKTTPPVLDSDGSFFLVSKLTVDKSRWQQGNVFSCSVMHE | |
| With C-terminal Lysine | ALHNHYTQKSLSLSPGK | |
| hIgG1 Hinge Region + | EPKSCDKTHTCPPCPAPELLGGPSVFLFPPKPKDTLMISRTPE | 3545 |
| CH2 Region + CH3 | VTCVVVDVSHEDPEVKFNWYVDGVEVHNAKTKPREEQYNSTYR | |
| Region | VVSVLTVLHQDWLNGKEYKCKVSNKALPAPIEKTISKAKGQPR | |
| Hole | EPQVCTLPPSRDELTKNQVSLSCAVKGFYPSDIAVEWESNGQP | |
| T366S/L368A/Y407V/Y349C | ENNYKTTPPVLDSDGSFFLVSKLTVDKSRWQQGNVFSCSVMHE | |
| Without C-terminal | ALHNHYTQKSLSLSPG | |
| Lysine | | |
| hIgG1 CH1 + Hinge | ASTKGPSVFPLAPSSKSTSGGTAALGCLVKDYFPEPVTVSWNS | 3546 |
| Region + CH2 Region + | GALISGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Hole | PKDTLMISRTPEVTCVVVDVSHEDPEVKFNWYVDGVEVHNAKT | |
| T366S/L368A/Y407V/Y349C | KPREEQYNSTYRVVSVLIVLHQDWLNGKEYKCKVSNKALPAPI | |
| With C-terminal Lysine | EKTISKAKGQPREPQVCTLPPSRDELTKNQVSLSCAVKGFYPS | |
| | DIAVEWESNGQPENNYKTTPPVLDSDGSFFLVSKLTVDKSRWQ | |
| | QGNVFSCSVMHEALHNHYTQKSLSLSPGK | |
| hIgG1 CH1 + Hinge | ASTKGPSVFPLAPSSKSTSGGTAALGCLVKDYFPEPVTVSWNS | 3547 |
| Region + CH2 Region + | GALTSGVHTFPAVLQSSGLYSLSSVVTVPSSSLGTQTYICNVN | |
| CH3 Region | HKPSNTKVDKKVEPKSCDKTHTCPPCPAPELLGGPSVFLFPPK | |
| Hole | PKDTLMISRTPEVTCVVVDVSHEDPEVKFNWYVDGVEVHNAKT | |
| T366S/L368A/Y407V/Y349C | KPREEQYNSTYRVVSVLIVLHQDWLNGKEYKCKVSNKALPAPI | |
| Without C-terminal | EKTISKAKGQPREPQVCTLPPSRDELTKNQVSLSCAVKGFYPS | |
| Lysine | DIAVEWESNGQPENNYKTTPPVLDSDGSFFLVSKLTVDKSRWQ | |
| | QGNVFSCSVMHEALHNHYTQKSLSLSPG | |

As described herein, in some embodiments, the antibody (or heterologous polypeptide) comprises a first Fc region and a second Ig Fc region.

In some embodiments, the amino acid sequence of the first Fc region comprises a T366W amino acid substitution, EU numbering according to Kabat; and the second the amino acid sequence of the Fc region comprises each of the following amino acid substitutions: T366S, L368A, and Y407V, EU numbering according to Kabat; each relative to the amino acid sequence of an exemplary reference Ig Fc region (e.g., a reference Ig Fc region set forth in Table 15). In some embodiments, the amino acid sequence of the first hIg further comprises a S354C amino acid substitution, EU numbering according to Kabat; and the amino acid sequence of the second Fc region comprises a Y349C amino acid substitution, EU numbering according to Kabat; each relative to the amino acid sequence of an exemplary reference Ig Fc region (e.g., a reference Ig Fc region set forth in Table 15).

In some embodiments, the amino acid sequence of the first Fc region comprises each of the following amino acid substitutions: T366W and a S354C, EU numbering according to Kabat; and the second the amino acid sequence of the Fc region comprises each of the following amino acid substitutions: T366S, L368A, Y407V, and Y349C, EU numbering according to Kabat; each relative to the amino acid sequence of an exemplary reference Ig Fc region (e.g., a reference Ig Fc region set forth in Table 15).

In some embodiments, the amino acid sequence of the second Fc region comprises a T366W amino acid substitution, EU numbering according to Kabat; and the second the amino acid sequence of the Fc region comprises each of the following amino acid substitutions: T366S, L368A, and Y407V, EU numbering according to Kabat; each relative to the amino acid sequence of an exemplary reference Ig Fc region (e.g., a reference Ig Fc region set forth in Table 15). In some embodiments, the amino acid sequence of the second hIg further comprises a S354C amino acid substitution, EU numbering according to Kabat; and the amino acid sequence of the second Fc region comprises a Y349C amino acid substitution, EU numbering according to Kabat; each relative to the amino acid sequence of an exemplary reference Ig Fc region (e.g., a reference Ig Fc region set forth in Table 15).

In some embodiments, the amino acid sequence of the second Fc region comprises each of the following amino acid substitutions: T366W and a S354C, EU numbering according to Kabat; and the second the amino acid sequence of the Fc region comprises each of the following amino acid substitutions: T366S, L368A, Y407V, and Y349C, EU numbering according to Kabat; each relative to the amino acid sequence of an exemplary reference Ig Fc region (e.g., a reference Ig Fc region set forth in Table 15).

In some embodiments, the amino acid sequence of the first Ig Fc region comprises a W amino acid at position T366, EU numbering according to Kabat; and the amino acid sequence of the second Ig Fc region comprises a S amino acid at position T366, an A amino acid at position L368, and a V amino acid at position Y407, EU numbering according to Kabat.

In some embodiments, the amino acid sequence of the first Ig Fc region comprises a W amino acid at position T366 and a C amino acid at position S354, EU numbering according to Kabat; and the amino acid sequence of the second Ig Fc region comprises a S amino acid at position T366, an A amino acid at position L368, a V amino acid at position Y407, and a C amino acid at position Y349, EU numbering according to Kabat.

### Ig Constant Region Variations for Site Specific Conjugation

In some embodiments, the agent (e.g., an antisense strand, sense strand, dsRNA agent, RNAi agent, ASO, ssRNA agent, etc. described herein) is conjugated (e.g., directly, or indirectly through a linker) to the Ig constant region. In some embodiments, the agent (e.g., an antisense strand, sense strand, dsRNA agent, RNAi agent, ASO, ssRNA agent, etc. described herein) (or a linker) is conjugated directly to an amino acid (e.g., a naturally occurring amino acid or an engineered (i.e., variant) amino acid) within the Ig constant region.

In some embodiments, the agent (e.g., an antisense strand, sense strand, dsRNA agent, RNAi agent, ASO, ssRNA agent, etc. described herein) (or the linker) is conjugated directly to an engineered lysine, cysteine, or tyrosine amino acid residue within the Ig constant region. In some embodiments, the amino acid sequence of the Ig constant region comprises the substitution of one or more naturally occurring amino acid residue with a lysine, cysteine, or tyrosine amino acid residue (e.g., to mediate conjugation). In some embodiments, the amino acid sequence of the Ig constant region comprises the substitution of one or more non-cysteine amino acid residue with a cysteine amino acid residue (e.g., to mediate conjugation). In some embodiments, the amino acid sequence of the Ig constant region comprises the substitution of one or more non-lysine amino acid residue with a lysine amino acid residue (e.g., to mediate conjugation). In some embodiments, the amino acid sequence of the Ig constant region comprises the substitution of one or more non-tyrosine amino acid residue with a tyrosine amino acid residue (e.g., to mediate conjugation).

In some embodiments, the amino acid sequence of the Ig constant region comprises the addition of one or more lysine, cysteine, or tyrosine amino acid residue (e.g., to mediate conjugation). In some embodiments, the amino acid sequence of the Ig constant region comprises the addition of one or more lysine amino acid residue (e.g., to mediate conjugation). In some embodiments, the amino acid sequence of the Ig constant region comprises the addition of one or more cysteine amino acid residue (e.g., to mediate conjugation). In some embodiments, the amino acid sequence of the Ig constant region comprises the addition of one or more tyrosine amino acid residue (e.g., to mediate conjugation).

### 4.5.2 Linkers

In some embodiments, the agent (e.g., described herein) (e.g., RNAi agent, dsRNA agent, sense strand, antisense strand, ASO, ssRNA agent, etc.) is directly attached to the heterologous moiety (e.g., targeting moiety) (e.g., directly attached through a single chemical bond). In some embodiments, the agent (e.g., described herein) (e.g., RNAi agent, dsRNA agent, sense strand, antisense strand, ASO, ssRNA agent, etc.) is indirectly attached to the heterologous moiety (e.g., targeting moiety). In some embodiments, the agent (e.g., described herein) (e.g., RNAi agent, dsRNA agent, sense strand, antisense strand, ASO, ssRNA agent, etc.) is indirectly attached to the heterologous moiety via a linker.

Suitable linkers for use in the conjugates described herein are known in the art and can be evaluated by a person of ordinary skill in the art using standard methods. Exemplary linkers and components thereof for use in the conjugates described herein are also described below.

Linkers typically comprise a direct bond or an atom such as oxygen or sulfur, a unit such as NR8, C(O), C(O)NH, SO, SO2, SO2NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, or substituted or unsubstituted heterocyclic; where R8 is hydrogen, acyl, aliphatic, or substituted aliphatic. In one embodiment, the linker is about 1-24 atoms, 2-24, 3-24, 4-24, 5-24, 6-24, 6-18, 7-18, 8-18, 7-17, 8-17, 6-16, 7-17, or 8-16 atoms.

In some embodiments, the linker comprises ethylene glycol, nucleosides, or amino acid units. In some embodiments, the linker comprises one or more groups selected from alkyl, amino, oxo, amide, disulfide, polyethylene glycol, ether, thioether, and hydroxylamino. In some embodiments, the linker comprises groups selected from alkyl, amino, oxo, amide and ether groups. In some embodiments, the linker comprises groups selected from alkyl and amide groups. In some embodiments, the linker comprises groups selected from alkyl and ether groups. In some embodiments, the linker comprises at least one phosphorus moiety. In some embodiments, the linker comprises at least one phosphate group. In some embodiments, the linker comprises at least one neutral linking group. Exemplary linkers include but are not limited to pyrrolidine, 8-amino-3,6-dioxaoctanoic acid (ADO), succinimidyl 4-(N-maleimidomethyl) cyclohexane-1-carboxylate (SMCC), 6-aminohexanoic acid (AHEX or AHA). Additional exemplary linkers include but are not limited to substituted or unsubstituted Ci-Cw alkyl, substituted or unsubstituted C2-C10 alkenyl or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In some embodiments, the linker is bifunctional. In general, a bifunctional linker comprises at least two functional groups. One of the functional groups is selected to react with a particular site on an agent (e.g., described herein) and the other is selected to react with a heterologous moiety (e.g., described herein). Examples of functional groups used in a bifunctional linkers include but are not limited to electrophiles for reacting with nucleophilic groups and nucleophiles for reacting with electrophilic groups. In some embodiments, bifunctional linking moieties comprise one or more groups selected from amino, hydroxyl, carboxylic acid, thiol, alkyl, alkenyl, and alkynyl.

In some embodiments, the linker is a monovalent linker, a bivalent linker, a trivalent linker, or a tetravalent linker.

### 4.5.2.1 Cleavable Linkers

In some embodiments, the linker is non-cleavable. In some embodiments, the linker is cleavable. Cleavable linkers contain at least one (or a plurality of) cleavable bonds that are susceptible to one or more cleavage agent. Exemplary classes of cleavable linkers include, but are not limited to, redox cleavable linkers, phosphate based cleavable linkers, acid cleavable linkers, ester-based cleavable linkers, and peptide-based cleavable linkers. In certain embodiments, a cleavable bond is selected from among: an amide, an ester, an ether, one or both esters of a phosphodiester, a phosphate ester, a carbamate, or a disulfide.

Cleavable linkers may be advantageous when a stable conjugate is desired under a first set of conditions but under a second set of conditions it is advantageous to release the agent (e.g., described herein) from the heterologous moiety (e.g., described herein). For example, in some embodiments, it may be desirable to have a sufficiently stable conjugate outside of a cell (e.g., within a subject (e.g., within the blood or serum of a subject)), and upon entry into a cell (e.g., a target cell (e.g., a target cell within a subject)) have the linker cleaved to release the agent (e.g., described herein) from the heterologous moiety (e.g., described herein). In some embodiments, the linker is not cleaved (or is cleaved at a lower rate) under a first condition relative to under a second condition. In some embodiments, the first condition is within the blood (e.g., of a subject) (or in an in vitro system sufficient to mimic the conditions of the blood within a subject) and the second condition is with a cell (e.g., a cell within a subject) (or in an in vitro system sufficient to mimic the conditions of a cell within a subject).

The suitability of a cleavable linker can be assessed by standard methods known in the art. In general, the suitability of a cleavable linker can be evaluated by testing the ability of a cleavage agent (or condition) to cleave the candidate linker (e.g., the cleavage bond(s)). In some embodiments, it may be desirable to further test the ability of the linker to resist cleavage under a certain condition (e.g., within the blood or serum of subject, when in contact with a non-target cell, tissue, organ).

In some embodiments, the linker is a redox cleavable linker that is cleaved upon reduction or oxidation. An example of a reductively cleavable linker is a disulphide (—S—S—) containing linker. Redox cleavable linkers can be evaluated using methods analogous to those described above.

In some embodiments, the linker is a phosphate-based cleavable linker. A phosphate-based cleavable linker is cleaved by agents that degrade or hydrolyze the phosphate group. For example, in cells, enzymes such as phosphatases are capable of cleaving phosphate groups. Examples of phosphate-based linkers include those comprising any of the following —O—P(O)(ORk)-O—, —OP(S)(ORk)-O—, —O—P(S)(SRk)-O—, —S—P(O)(ORk)-O—, —O—P(O)(ORk)-S—, —S—P(O)(ORk)-S—, —OP(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—, wherein Rk at each occurrence can be, independently, C1-C20 alkyl, C1-C20 haloalkyl, C6-C10 aryl, or C7-C12 aralkyl. Exemplary embodiments include are —OP(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O, —S—P(S)(H)—O—, —S—P(O)(H)—S—, or —O—P(S)(H)—S—. Phosphate based cleavable linker can be evaluated using methods analogous to those described above.

In some embodiments, the linker is an acid cleavable linker. An acid cleavable linker is cleaved under acidic conditions. For example, in some embodiments the acid cleavable linker can be cleaved in an acidic environment with a pH of about 6.5 or less (e.g., about 6.0, 5.5, 5.0, or less). In some embodiments the acid cleavable linker can be cleaved by enzymes that can act as a general acid. In a cell (e.g., within a subject), specific low pH organelles, such as endosomes and lysosomes can provide a cleaving environment for acid cleavable linkers. Examples of acid cleavable linkers include but are not limited to hydrazones, esters, and esters of amino acids. Acid cleavable groups can have the general formula —C═NN—, C(O)O, or —OC(O). Acid cleavable linkers can be evaluated using methods analogous to those described above.

In some embodiments, the linker is an ester-based cleavable linker. An ester-based cleavable linker is cleaved by enzymes such as esterases and amidases in cells. Examples of ester-based cleavable include, but are not limited to, esters of alkylene, alkenylene and alkynylene groups. The cleavable bonds of ester cleavable linkers have the general formula —C(O)O— or —OC(O)—. Ester-based cleavable linkers can be evaluated using methods analogous to those described above.

In some embodiments, the linker is a peptide-based cleavable linker. A peptide-based cleavable linker is cleaved by enzymes such as peptidases and proteases (e.g., present in cells (e.g., cells within a subject)). Peptide-based cleavable linkers comprise peptide bonds formed between amino acids to yield polypeptides (e.g., dipeptides, tripeptides, etc.). As known in the art, peptide bonds. The peptide bonds (i.e., the amide bond) of the peptide linker is generally the site of cleavage. Peptide-based cleavable linkers can be evaluated using methods analogous to those described above.

### 4.5.3 Orientation

The heterologous moiety may be attached at any suitable position to the agent (e.g., described herein) (e.g., RNAi agent, dsRNA agent, sense strand, antisense strand, ASO, ssRNA agent, etc.).

In some embodiments, the heterologous moiety is conjugated to the 5′ end of the agent (e.g., described herein) (e.g., RNAi agent, dsRNA agent, sense strand, antisense strand, ASO, ssRNA agent, etc.). In some embodiments, the heterologous moiety is conjugated to the 3′ end of the agent (e.g., described herein) (e.g., RNAi agent, dsRNA agent, sense strand, antisense strand, ASO, ssRNA agent, etc.). In some embodiments, a first heterologous moiety is conjugated to the 5′ end of the agent (e.g., described herein) (e.g., RNAi agent, dsRNA agent, sense strand, antisense strand, ASO, ssRNA agent, etc.) and a second heterologous moiety is conjugated to the 3′ end of the agent (e.g., described herein) (e.g., RNAi agent, dsRNA agent, sense strand, antisense strand, ASO, ssRNA agent, etc.). The first and second heterologous moieties can be the same or different. In some embodiments, the heterologous moiety is conjugated to an internal site of the agent (e.g., described herein) (e.g., RNAi agent, dsRNA agent, sense strand, antisense strand, ASO, ssRNA agent, etc.).

In some embodiments, the agent (e.g., RNAi agent, dsRNA agent) comprises an antisense strand. In some embodiments, the heterologous moiety is conjugated to the 5′ end of the antisense strand. In some embodiments, the heterologous moiety is conjugated to the 3′ end of the antisense strand. In some embodiments, a first heterologous moiety is conjugated to the 5′ end of the antisense strand and a second heterologous moiety is conjugated to the 3′ end of the antisense strand. The first and second heterologous moieties can be the same or different. In some embodiments, the heterologous moiety is conjugated to an internal site of the antisense strand.

In some embodiments, the agent (e.g., RNAi agent, dsRNA agent) comprises a sense strand. In some embodiments, the heterologous moiety is conjugated to the 5′ end of the sense strand. In some embodiments, the heterologous moiety is conjugated to the 3′ end of the sense strand. In some embodiments, a first heterologous moiety is conjugated to the 5′ end of the sense strand and a second heterologous moiety is conjugated to the 3′ end of the sense strand. The first and second heterologous moieties can be the same or different. In some embodiments, the heterologous moiety is conjugated to an internal site of the sense strand.

The heterologous moiety may be attached to the 3′ end of the sense and/or antisense strand. The heterologous moiety may be attached to the 5′ end of the sense and/or antisense strand. The heterologous moiety may be attached to at an internal site of the sense and/or antisense strand. The heterologous moiety may be attached to the 3′ end of the sense and antisense strand. The heterologous moiety may be attached to the 5′ end of the sense and antisense strand. The heterologous moiety may be attached to at an internal site of the sense and antisense strand.

In some embodiments, the agent (e.g., RNAi agent) comprises a dsRNA agent comprising a sense strand and an antisense strand. In some embodiments, the heterologous moiety is conjugated to the 5′ end of the sense strand. In some embodiments, the heterologous moiety is conjugated to the 3′ end of the sense strand. In some embodiments, a first heterologous moiety is conjugated to the 5′ end of the sense strand and a second heterologous moiety is conjugated to the 3′ end of the sense strand. The first and second heterologous moieties can be the same or different. In some embodiments, the heterologous moiety is conjugated to an internal site of the sense strand. In some embodiments, the heterologous moiety is conjugated to the 5′ end of the sense strand. In some embodiments, the heterologous moiety is conjugated to the 3′ end of the sense strand. In some embodiments, a first heterologous moiety is conjugated to the 5′ end of the sense strand and a second heterologous moiety is conjugated to the 3′ end of the sense strand. The first and second heterologous moieties can be the same or different. In some embodiments, the heterologous moiety is conjugated to an internal site of the sense strand.

In some embodiments, a first heterologous moiety is conjugated to the 5′ end of the sense strand and a second heterologous moiety is conjugated to the 5′ end of the antisense strand. In some embodiments, a first heterologous moiety is conjugated to the 3′ end of the sense strand and a second heterologous moiety is conjugated to the 3′ end of the antisense strand. In some embodiments, a first heterologous moiety is conjugated to the 5′ end of the sense strand and a second heterologous moiety is conjugated to the 3′ end of the antisense strand. In some embodiments, a first heterologous moiety is conjugated to the 3′ end of the sense strand and a second heterologous moiety is conjugated to the 5′ end of the antisense strand. The first and second heterologous moieties can be the same or different.

In some embodiments, a first heterologous moiety is conjugated to an internal site of the sense strand and a second heterologous moiety is conjugated to the 5′ end of the antisense strand. In some embodiments, a first heterologous moiety is conjugated to an internal site of the sense strand and a second heterologous moiety is conjugated to the 3′ end of the antisense strand. In some embodiments, a first heterologous moiety is conjugated to an internal site of the antisense strand and a second heterologous moiety is conjugated to the 3′ end of the antisense strand. In some embodiments, a first heterologous moiety is conjugated to an internal site of the antisense strand and a second heterologous moiety is conjugated to the 5′ end of the antisense strand. The first and second heterologous moieties can be the same or different.

### 4.6 Activity of Nucleic Acis Molecule Agents & Conjugates Thereof

In some embodiments, the agent (e.g., dsRNA agent, RNAi agent, ASO, ssRNA agent, etc.) inhibits expression of a target gene (e.g., BCL11A). In some embodiments, the agent (e.g., dsRNA agent, RNAi agent, ASO, ssRNA agent, etc.) inhibits expression of a target gene (e.g., BCL11A) in a cell. In some embodiments, the agent (e.g., dsRNA agent, RNAi agent, ASO, ssRNA agent, etc.) inhibits expression of a target gene (e.g., BCL11A) in a cell in a subject (e.g., a mammalian subject, e.g., a primate, human, non-human primate, mouse, rat, etc.). In some embodiments, the agent (e.g., dsRNA agent, RNAi agent, ASO, ssRNA agent, etc.) inhibits expression of a target gene (e.g., BCL11A) in a cell in a subject (e.g., a mammalian subject, e.g., a primate, human, non-human primate, mouse, rat, etc.) by at least about 30%, 40%, 50%, 55%, 60%, 65%, 70%, 75%, 80%, 85%, 90%, 95%, 96%, 97%, 98%, 99%, or 100%. In some embodiments, the agent (e.g., dsRNA agent, RNAi agent, ASO, ssRNA agent, etc.) mediates degradation of a target mRNA (e.g., a BCL11A (e.g., hBCL11A) mRNA). In some embodiments, the agent (e.g., dsRNA agent, RNAi agent, ASO, ssRNA agent, etc.) inhibits expression of a target gene (e.g., BCL11A) in a cell in a subject (e.g., a mammalian subject, e.g., a primate, human, non-human primate, mouse, rat, etc.) by at least about 50%, 55%, 60%, 65%, 70%, 75%, 80%, 85%, 90%, 95%, 96%, 97%, 98%, 99%, or 100%. In some embodiments, the agent (e.g., dsRNA agent, RNAi agent, ASO, ssRNA agent, etc.) inhibits expression of a target gene (e.g., BCL11A) in a cell in a subject (e.g., a mammalian subject, e.g., a primate, human, non-human primate, mouse, rat, etc.) by at least about 75%, 80%, 85%, 90%, 95%, 96%, 97%, 98%, 99%, or 100%. In some embodiments, the agent (e.g., dsRNA agent, RNAi agent, ASO, ssRNA agent, etc.) inhibits expression of a target gene (e.g., BCL11A) in a cell in a subject (e.g., a mammalian subject, e.g., a primate, human, non-human primate, mouse, rat, etc.) by at least about 90%, 95%, 96%, 97%, 98%, 99%, or 100%. In some embodiments, the agent (e.g., dsRNA agent, RNAi agent, ASO, ssRNA agent, etc.) inhibits expression of a target gene (e.g., BCL11A) in a cell in a subject (e.g., a mammalian subject, e.g., a primate, human, non-human primate, mouse, rat, etc.) by at least about 50%. In some embodiments, the agent (e.g., dsRNA agent, RNAi agent, ASO, ssRNA agent, etc.) inhibits expression of a target gene (e.g., BCL11A) in a cell in a subject (e.g., a mammalian subject, e.g., a primate, human, non-human primate, mouse, rat, etc.) by at least about 75%. In some embodiments, the agent (e.g., dsRNA agent, RNAi agent, ASO, ssRNA agent, etc.) inhibits expression of a target gene (e.g., BCL11A) in a cell in a subject (e.g., a mammalian subject, e.g., a primate, human, non-human primate, mouse, rat, etc.) by at least about 80%. In some embodiments, the agent (e.g., dsRNA agent, RNAi agent, ASO, ssRNA agent, etc.) inhibits expression of a target gene (e.g., BCL11A) in a cell in a subject (e.g., a mammalian subject, e.g., a primate, human, non-human primate, mouse, rat, etc.) by at least about 90%. In some embodiments, the agent (e.g., dsRNA agent, RNAi agent, ASO, ssRNA agent, etc.) inhibits expression of a target gene (e.g., BCL11A) in a cell in a subject (e.g., a mammalian subject, e.g., a primate, human, non-human primate, mouse, rat, etc.) by at least about 95%.

Any one or more of the above activities can be evaluated in vitro, ex vivo, or in vivo. Any one or more of the above activities can be evaluated by standard methods known in the art. For example, by PCR (e.g., qPCR), branched DNA assays, or by a protein-based methods (such as immunofluorescence analysis (using, e.g., western blotting or flow cytometric techniques). In some embodiments, inhibition of gene (e.g., BCL11A (e.g., hBCL11A)) expression is determined by qPCR.

### 4.7 Methods of Making Nucleic Acid Molecule Agents & Conjugates Thereof

An agent (e.g., described herein) (e.g., RNAi agent, dsRNA agent, antisense strand, sense strand, ASO, ssRNA agent, etc.) can be synthesized by standard methods known in the art (e.g., chemical synthesis (e.g., solid phase synthesis)). See, e.g., “Current protocols in nucleic acid chemistry,” Beaucage, S. L. et al. (Edrs.), John Wiley & Sons, Inc., New York, N.Y., USA, and See, e.g., Dong Y, Siegwart D J, Anderson D G. Strategies, design, and chemistry in siRNA delivery systems. Adv Drug Deliv Rev. 2019 April; 144:133-147. doi: 10.1016/j.addr.2019.05.004. Epub 2019 May 15. PMID: 31102606; PMCID: PMC6745264, the entire contents of each of which is incorporated by reference herein for all purposes. As such, further provided herein are methods of making an agent described herein) (e.g., RNAi agent, dsRNA agent, antisense strand, sense strand, ASO, ssRNA agent, etc.).

For example, single stranded nucleic acid molecules (e.g., described herein) (e.g., antisense strands, sense strands, ASOs, ssRNA agents, etc.) can be prepared using solution-phase or solid-phase organic synthesis or both. dsRNA agents (e.g., described herein) can be prepared using a two-step procedure, wherein the individual strands of the dsRNA agent are prepared separately and subsequently annealed. The individual strands of the dsRNA agent can be prepared using solution-phase or solid-phase organic synthesis or both. Regardless of the method of synthesis, the agents (e.g., described herein) (e.g., dsRNA agents described herein) can be prepared in a solution (e.g., an aqueous or organic solution) that is appropriate for formulation. For example, the dsRNA agent can be precipitated and redissolved in pure double-distilled water, and lyophilized. The lyophilized dsRNA agent can be resuspended in a solution appropriate for the intended formulation process.

Likewise, conjugates (e.g., described herein) can be synthesized utilizing standard methods known in the art. See, e.g., Dong Y, Siegwart D J, Anderson D G. Strategies, design, and chemistry in siRNA delivery systems. Adv Drug Deliv Rev. 2019 April; 144:133-147. doi: 10.1016/j.addr.2019.05.004. Epub 2019 May 15. PMID: 31102606; PMCID: PMC6745264, the entire contents of which is incorporated herein by reference for all purposes. A person of ordinary skill in the art can determine the appropriate conjugation method based on e.g., the heterologous moiety and the agent to be conjugated. For example, standard conjugation methods include, e.g., parallel synthesis methods and linear synthesis methods.

### 4.8 Methods of Making Proteins (e.g., Targeting Moieties)

Any of the proteins (e.g., antibodies), peptides, fusion proteins, etc., including the targeting moieties described herein, may be produced using standard methods known in the art. For example, each may be produced by recombinant technology in host cells (e.g., insect cells, mammalian cells, bacteria) that have been transfected or transduced with a nucleic acid expression vector (e.g., plasmid, viral vector (e.g., a baculoviral expression vector)) encoding the protein (e.g., antibody). Such general methods are common knowledge in the art. The expression vector typically contains an expression cassette that includes nucleic acid sequences capable of bringing about expression of the nucleic acid molecule encoding the protein interest, such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the protein of interest are cultured under conditions conducive to expression of the nucleic acid molecule encoding the protein. Culture media is available from various vendors, and a suitable medium can be routinely chosen for a host cell to express a protein or polypeptide of interest. Host cells can be adherent or suspension cultures, and a person of ordinary skill in the art can optimize culture methods for specific host cells selected. For example, suspension cells can be cultured in, for example, bioreactors in e.g., a batch process or a fed-batch process. The produced protein or polypeptide may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The protein or polypeptide may be concentrated, buffer exchanged by ultrafiltration, and the retentate from the ultrafiltration may be filtered through an appropriate filter, e.g., a 0.22 μm filter. See, e.g., Hacker, David (Ed.), Recombinant Protein Expression in Mammalian Cells: Methods and Protocols (Methods in Molecular Biology), Humana Press (2018); and McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. No. 5,762,939, the entire contents of each of which is incorporated by reference herein for all purposes.

The proteins (e.g., antibodies), peptides, fusion proteins, etc. described herein may also be produced synthetically.

### 4.9 Vectors

In some embodiments, one or more of the agents described herein (e.g., RNAi agents, dsRNA agents, sense strands, antisense strands, ASOs, ssRNA agents, etc.) (see, e.g., §§ 4.2, 4.3, 4.4) are contained in a vector (e.g., a non-viral vector (e.g., a plasmid), a viral vector). Thus, in one aspect, also provided herein are vectors (e.g., non-viral vectors (e.g., plasmids) viral vectors) comprising one or more agent described herein (e.g., RNAi agents, dsRNA agents, sense strands, antisense strands, ASOs, ssRNA agents, etc.) (see, e.g., §§ 4.2, 4.3, 4.4). Such vectors can be easily manipulated by methods well known to the ordinary person of skill in the art. The vector used can be any vector that is suitable for cloning nucleic acid molecules that can be used for transcription of the nucleic acid molecule of interest (e.g., an agent described herein (e.g., RNAi agents, dsRNA agents, sense strands, antisense strands, ASOs, ssRNA agents, etc.) (see, e.g., §§ 4.2, 4.3, 4.4).

In some embodiments, the vector is a viral vector. Viral vectors include both RNA and DNA based vectors. The vectors can be designed to meet a variety of specifications. For example, viral vectors can be engineered to be capable or incapable of replication in prokaryotic and/or eukaryotic cells. In some embodiments, the vector is replication deficient. In some embodiments, the vector is replication competent. Vectors can be engineered or selected that either will (or will not) integrate in whole or in part into the genome of host cells, resulting (or not (e.g., episomal expression)) in stable host cells comprising the desired nucleic acid in their genome.

Exemplary viral vectors include, but are not limited to, adenovirus vectors, adeno-associated virus vectors, lentivirus vectors, retrovirus vectors, poxvirus vectors, parapoxvirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, anellovectors, or lymphocytic choriomeningitis virus vectors. In some embodiments, the viral vector is an adenovirus vector, adeno-associated virus vector, lentivirus vector, anellovector (as described, for example, in U.S. Pat. No. 11,446,344, the entire contents of which is incorporated by reference herein for all purposes).

In some embodiments, the vector is an adenoviral vector (e.g., human adenoviral vector, e.g., HAdV or AdHu). In some embodiments, the adenovirus vector has the E1 region deleted, rendering it replication-deficient in human cells. Other regions of the adenovirus such as E3 and E4 may also be deleted. Exemplary adenovirus vectors include, but are not limited to, those described in e.g., WO2005071093 or WO2006048215, the entire contents of each of which is incorporated by reference herein for all purposes. In some embodiments, the adenovirus-based vector used is a simian adenovirus, thereby avoiding dampening of the immune response after vaccination by pre-existing antibodies to common human entities such as AdHu5. Exemplary, simian adenovirus vectors include AdCh63 (see, e.g., WO2005071093, the entire contents of which is incorporated by reference herein for all purposes) or AdCh68.

Viral vectors can be generated through the use of a packaging/producer cell line (e.g., a mammalian cell line) using standard methods known to the person of ordinary skill in the art. Generally, a nucleic acid construct (e.g., a plasmid) encoding the transgene (e.g., an agent described herein) (along with additional elements e.g., a promoter, inverted terminal repeats (ITRs) flanking the transgene, a plasmid encoding e.g., viral replication and structural proteins, along with one or more helper plasmids a host cell (e.g., a host cell line) are transfected into a host cell line (i.e., the packing/producer cell line). In some instances, depending on the viral vector, a helper plasmid may also be needed that include helper genes from another virus (e.g., in the instance of adeno-associated viral vectors). Eukaryotic expression plasmids are commercially available from a variety of suppliers, for example the plasmid series: pcDNA™, pCR3.1™, pCMV™, pFRT™, pVAX1™, pCI™, Nanoplasmid™, and Pcaggs. The person of ordinary skill in the art is aware of numerous transfection methods and any suitable method of transfection may be employed (e.g., using a biochemical substance as carrier (e.g., lipofectamine), by mechanical means, or by electroporation). The cells are cultured under conditions suitable and for a sufficient time for plasmid expression. The viral particles may be purified from the cell culture medium using standard methods known to the person of ordinary skill in the art. For example, by centrifugation followed by e.g., chromatography or ultrafiltration.

In some embodiments, the vector is a plasmid. A person of ordinary skill in the art is aware of suitable plasmids for expression of the DNA of interest. For example, Suitable plasmid DNA may be generated to allow efficient production of the encoded RNA in cell lines, e.g., in insect cell lines, for example using vectors as described in W02009150222A2 and as defined in PCT claims 1 to 33, the disclosure relating to claim 1 to 33 of W02009150222A2 the entire contents of which is incorporated by reference herein for all purposes.

### 4.10 Carriers

In some embodiments, one or more of the agents described herein (e.g., RNAi agents, dsRNA agents, sense strands, antisense strands, ASOs, ssRNA agents, etc. (or a conjugate comprising the same)) or a vector comprising any of the foregoing is formulated within one or more carrier.

Therefore, further provided herein are carriers comprising any one or more of the agents described herein (e.g., RNAi agents, dsRNA agents, sense strands, antisense strands, ASOs, ssRNA agents, etc. (or a conjugate comprising the same)) or a vector comprising any of the foregoing.

Any of the foregoing (e.g., one or more of the agents described herein (e.g., RNAi agents, dsRNA agents, sense strands, antisense strands, ASOs, ssRNA agents, etc. (or a conjugate comprising the same)) or a vector comprising any of the foregoing) can be encapsulated within a carrier, chemically conjugated to a carrier, associated with the carrier. In this context, the term “associated” refers to the essentially stable combination of an agent described herein (or a conjugate comprising the same) (or a vector comprising the same) with one or more molecules of a carrier (e.g., one or more lipids of a lipid-based carrier, e.g., an LNP, liposome, lipoplex, and/or nanoliposome) into larger complexes or assemblies without covalent binding. In this context, the term “encapsulation” refers to the incorporation of an agent described herein (or a conjugate comprising the same) (or a vector comprising the same) into a carrier (e.g., a lipid-based carrier, e.g., an LNP, liposome, lipoplex, and/or nanoliposome) wherein the agent described herein (or the conjugate comprising the same) (or the vector comprising the same) is entirely contained within the interior space of the carrier (e.g., the lipid-based carrier, e.g., the LNP, liposome, lipoplex, and/or nanoliposome).

Exemplary carriers includes, but are not limited to, lipid-based carriers (e.g., lipid nanoparticles (LNPs), liposomes, lipoplexes, and nanoliposomes). In some embodiments, the carrier is a lipid-based carrier. In some embodiments, the carrier is an LNP. In some embodiments, the LNP comprises a cationic lipid, a neutral lipid, a cholesterol, and/or a PEG lipid.

### 4.11 Host Cells

In one aspect, provided herein are host cells comprising any one more agent described herein (e.g., an RNAi agent, a dsRNA agent, a sense strand, an antisense strand, an ASO, a ssRNA agent, etc.) (see, e.g., §§ 4.2, 4.3, 4.4); a vector described herein (see, e.g., § 4.9); a conjugate described herein (see, e.g., § 4.5); a carrier described herein (see, e.g., § 4.10); or any combination thereof. In some embodiments, the host cell comprises an RNAi agent described herein, a dsRNA agent described herein, an antisense strand described herein, and/or a sense strand described herein. In some embodiments, the host cell comprises a dsRNA agent described herein. In some embodiments, the host cell comprises an antisense oligonucleotide described herein. In some embodiments, the host cell is in vitro, ex vivo, or in vivo.

### 4.12 Pharmaceutical Compositions

In one aspect, provided herein are pharmaceutical compositions comprising any one more agent described herein (e.g., an RNAi agent, a dsRNA agent, a sense strand, an antisense strand, an ASO, a ssRNA agent, etc.) (see, e.g., §§ 4.2, 4.3, 4.4); a vector described herein (see, e.g., § 4.9); a conjugate described herein (see, e.g., § 4.5); a carrier described herein (see, e.g., § 4.10); and/or a host cell described herein (see, e.g., § 4.11); or any combination thereof; and a pharmaceutically acceptable excipient (see, e.g., Remington's Pharmaceutical Sciences (1990) Mack Publishing Co., Easton, PA, the entire contents of which is incorporated by reference herein for all purposes).

In one aspect, also provided herein are methods of making pharmaceutical compositions described herein comprising providing any one more agent described herein (e.g., an RNAi agent, a dsRNA agent, a sense strand, an antisense strand, an ASO, a ssRNA agent, etc.) (see, e.g., §§ 4.2, 4.3, 4.4); a vector described herein (see, e.g., § 4.9); a conjugate described herein (see, e.g., § 4.5); a carrier described herein (see, e.g., § 4.10); and/or a host cell described herein (see, e.g., § 4.11); and formulating it into a pharmaceutically acceptable composition by the addition of one or more pharmaceutically acceptable excipient.

Also provided herein are pharmaceutical compositions comprising any one more agent described herein (e.g., an RNAi agent, a dsRNA agent, a sense strand, an antisense strand, an ASO, a ssRNA agent, etc.); vector described herein; conjugate described herein; carrier described herein; and/or cell described herein, wherein the pharmaceutical composition lacks a predetermined threshold amount or a detectable amount of a process impurity or contaminant, e.g., lacks a predetermined threshold amount or a detectable amount of a process-related impurity such as host cell proteins, host cell DNA, or a cell culture component (e.g., inducers, antibiotics, or media components); a product-related impurity (e.g., precursors, fragments, aggregates, degradation products); or a contaminant, e.g., endotoxin, bacteria, viral contaminant.

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

A pharmaceutical composition may be formulated for any route of administration to a subject. Non-limiting embodiments include parenteral administration, such as intramuscular, intradermal, subcutaneous, transcutaneous, or mucosal.

In one embodiment, the pharmaceutical composition is formulated for administration by intramuscular, intradermal, or subcutaneous injection. In one embodiment, the pharmaceutical composition is formulated for administration by intramuscular injection. In one embodiment, the pharmaceutical composition is formulated for administration by intradermal injection. In one embodiment, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; or sodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 4.13 Methods of Use

Provided herein are various methods of utilizing any one more agent described herein (e.g., an RNAi agent, a dsRNA agent, a sense strand, an antisense strand, an ASO, a ssRNA agent, etc.) (see, e.g., §§ 4.2, 4.3, 4.4); a vector described herein (see, e.g., § 4.9); a conjugate described herein (see, e.g., § 4.5); a carrier described herein (see, e.g., § 4.10); a host cell described herein (see, e.g., § 4.11); and/or a pharmaceutical composition described herein (see, e.g., § 4.12); or any combination thereof.

In some aspects, the methods described herein comprise administering one or more of the foregoing to a subject. Exemplary subjects include mammals, e.g., humans, non-human mammals, e.g., non-human primates. In some embodiments, the subject is a human.

The dosage of any of the foregoing, to be administered to a subject in accordance with any of the methods described herein can be determined in accordance with standard techniques known to those of ordinary skill in the art, including the route of administration, the age and weight of the subject.

### 4.13.1 Methods of Delivery

Provided herein are, inter alia, various methods of delivering any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein; or any combination thereof to e.g., a cell, subject, a cell within a subject.

In one aspect, provided herein are methods of delivering any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein; or any combination thereof to a cell, the method comprising introducing into a cell any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein into the cell, to thereby deliver the agent (e.g., RNAi agent, dsRNA agent, sense strand, antisense strand, ASO, ssRNA agent, etc.), conjugate, vector, carrier, or pharmaceutical composition into the cell. In some embodiments, the agent (e.g., RNAi agent, dsRNA agent, sense strand, antisense strand, ASO, ssRNA agent, etc.), conjugate, vector, carrier, or pharmaceutical composition is introduced in an amount and for a time sufficient to deliver the agent (e.g., RNAi agent, dsRNA agent, sense strand, antisense strand, ASO, ssRNA agent, etc.), conjugate, vector, carrier, or pharmaceutical composition into the cell. In some embodiments, the cell is in vitro, ex vivo, or in vivo. In some embodiments, the cell is in vitro or ex vivo. In some embodiments, the cell is in vitro. In some embodiments, the cell is ex vivo. In some embodiments, the cell is in vivo. In some embodiments, the cells is a hematopoietic cell. In some embodiments, the cells is an erythroid precursor cell.

In one aspect, provided herein are methods of delivering any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein; or any combination thereof to a subject, the method comprising administering to a subject any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein into the cell, to thereby deliver the agent (e.g., RNAi agent, dsRNA agent, sense strand, antisense strand, ASO, ssRNA agent, etc.), conjugate, vector, carrier, or pharmaceutical composition to the subject. In some embodiments, the agent (e.g., RNAi agent, dsRNA agent, sense strand, antisense strand, ASO, ssRNA agent, etc.), conjugate, vector, carrier, or pharmaceutical composition is administered in an amount and for a time sufficient to deliver the agent (e.g., RNAi agent, dsRNA agent, sense strand, antisense strand, ASO, ssRNA agent, etc.), conjugate, vector, carrier, or pharmaceutical composition to the subject. In some embodiments, the cells is a hematopoietic cell. In some embodiments, the cells is an erythroid precursor cell.

In one aspect, provided herein are methods of delivering any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein; or any combination thereof to a cell within a subject, the method comprising administering to a cell within a subject any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein into the cell, to thereby deliver the agent (e.g., RNAi agent, dsRNA agent, sense strand, antisense strand, ASO, ssRNA agent, etc.), conjugate, vector, carrier, or pharmaceutical composition to the cell within the subject. In some embodiments, the agent (e.g., RNAi agent, dsRNA agent, sense strand, antisense strand, ASO, ssRNA agent, etc.), conjugate, vector, carrier, or pharmaceutical composition is administered in an amount and for a time sufficient to deliver the agent (e.g., RNAi agent, dsRNA agent, sense strand, antisense strand, ASO, ssRNA agent, etc.), conjugate, vector, carrier, or pharmaceutical composition to the cell within the subject. In some embodiments, the cells is a hematopoietic cell. In some embodiments, the cells is an erythroid precursor cell.

### 4.13.2 Methods of Reducing or Inhibiting BCL11A Expression

In one aspect, provided herein are methods of reducing or inhibiting expression of BCL11A (e.g., hBCL11A) in a cell, the method comprising introducing into the cell any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein, to thereby reduce or inhibit expression of BCL11A (e.g., hBCL11A) in the cell. In some embodiments, the agent (e.g., RNAi agent, dsRNA agent, sense strand, antisense strand, ASO, ssRNA agent, etc.), conjugate, vector, carrier, or pharmaceutical composition is introduced in an amount and for a time sufficient to reduce or inhibit expression of BCL11A (e.g., hBCL11A) in the cell. In some embodiments, the cell is in vitro, ex vivo, or in vivo. In some embodiments, the cell is in vitro or ex vivo. In some embodiments, the cell is in vitro. In some embodiments, the cell is ex vivo. In some embodiments, the cell is in vivo. In some embodiments, the cells is a hematopoietic cell. In some embodiments, the cells is an erythroid precursor cell.

In some embodiments, the level of BCL11A (e.g., hBCL11A) expression in the cell is reduced by at least about 30%, 40%, 50%, 55%, 60%, 65%, 70%, 75%, 80%, 85%, 90%, 95%, 96%, 97%, 98%, 99%, or 100% (e.g., as compared to suitable control) (e.g., as measured by an assay known in the art or described herein, e.g., qPCR). In some embodiments, the level of BCL11A (e.g., hBCL11A) expression in the cell is reduced by at least about 50% (e.g., as compared to suitable control) (e.g., as measured by an assay known in the art or described herein, e.g., qPCR). In some embodiments, the level of BCL11A (e.g., hBCL11A) expression in the cell is reduced by at least about 75% (e.g., as compared to suitable control) (e.g., as measured by an assay known in the art or described herein, e.g., qPCR). In some embodiments, the level of BCL11A (e.g., hBCL11A) expression in the cell is reduced by at least about 80% (e.g., as compared to suitable control) (e.g., as measured by an assay known in the art or described herein, e.g., qPCR). In some embodiments, the level of BCL11A (e.g., hBCL11A) expression in the cell is reduced by at least about 90% (e.g., as compared to suitable control) (e.g., as measured by an assay known in the art or described herein, e.g., qPCR). In some embodiments, the level of BCL11A (e.g., hBCL11A) expression in the cell is reduced by at least about 95% (e.g., as compared to suitable control) (e.g., as measured by an assay known in the art or described herein, e.g., qPCR).

In one aspect, provided herein are methods of reducing or inhibiting expression of BCL11A (e.g., hBCL11A) in a cell in a subject, the method comprising administering to a subject any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein, to thereby reduce or inhibit expression of BCL11A (e.g., hBCL11A) in the cell in the subject. In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to reduce or inhibit expression of BCL11A (e.g., hBCL11A) in the cell in the subject. In some embodiments, the level of BCL11A (e.g., hBCL11A) expression in the cell is reduced by at least about 30%, 40%, 50%, 55%, 60%, 65%, 70%, 75%, 80%, 85%, 90%, 95%, 96%, 97%, 98%, 99%, or 100% (e.g., as compared to suitable control) (e.g., as measured by an assay known in the art or described herein, e.g., qPCR). In some embodiments, the cells is a hematopoietic cell. In some embodiments, the cells is an erythroid precursor cell.

In one aspect, provided herein are dsRNA agents, conjugates, vectors, carriers, and pharmaceutical compositions for use in a method of reducing or inhibiting expression of BCL11A (e.g., hBCL11A) in a cell in a subject, the method comprising administering to the subject a dsRNA agent described herein, a conjugate described herein, a vector described herein, a carrier described herein, or a pharmaceutical composition described herein, to thereby reduce or inhibit expression of BCL11A (e.g., hBCL11A) in the cell in the subject. In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to reduce or inhibit expression of BCL11A (e.g., hBCL11A) in the cell in the subject. In some embodiments, the level of BCL11A (e.g., hBCL11A) expression in the cell is reduced by at least about 30%, 40%, 50%, 55%, 60%, 65%, 70%, 75%, 80%, 85%, 90%, 95%, 96%, 97%, 98%, 99%, or 100% (e.g., as compared to suitable control) (e.g., as measured by an assay known in the art or described herein, e.g., qPCR). In some embodiments, the cells is a hematopoietic cell. In some embodiments, the cells is an erythroid precursor cell.

In one aspect, provided herein are uses of dsRNA agents, conjugates, vectors, carriers, and pharmaceutical compositions in the manufacture of a medicament for the reduction or inhibition of BCL11A (e.g., hBCL11A) expression in a cell in a subject. In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to reduce or inhibit expression of BCL11A (e.g., hBCL11A) in the cell in the subject. In some embodiments, the level of BCL11A (e.g., hBCL11A) expression in the cell is reduced by at least about 30%, 40%, 50%, 55%, 60%, 65%, 70%, 75%, 80%, 85%, 90%, 95%, 96%, 97%, 98%, 99%, or 100% (e.g., as compared to suitable control) (e.g., as measured by an assay known in the art or described herein, e.g., qPCR). In some embodiments, the cells is a hematopoietic cell. In some embodiments, the cells is an erythroid precursor cell.

In one aspect, provided herein are uses of dsRNA agents, conjugates, vectors, carriers, and pharmaceutical compositions in the manufacture of a medicament for use in a method of reducing or inhibiting expression of BCL11A (e.g., hBCL11A) in a cell in a subject, the method comprising administering to the subject a dsRNA agent described herein, a conjugate described herein, a vector described herein, a carrier described herein, or a pharmaceutical composition described herein, to thereby reduce or inhibit expression of BCL11A (e.g., hBCL11A) in the cell in the subject. In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to reduce or inhibit expression of BCL11A (e.g., hBCL11A) in the cell in the subject. In some embodiments, the level of BCL11A (e.g., hBCL11A) expression in the cell is reduced by at least about 30%, 40%, 50%, 55%, 60%, 65%, 70%, 75%, 80%, 85%, 90%, 95%, 96%, 97%, 98%, 99%, or 100% (e.g., as compared to suitable control) (e.g., as measured by an assay known in the art or described herein, e.g., qPCR). In some embodiments, the cells is a hematopoietic cell. In some embodiments, the cells is an erythroid precursor cell.

### 4.13.3 Methods of Inducing Expression of Fetal Hemoglobin

In one aspect, provided herein are methods of inducing expression of increasing the level of fetal hemoglobin in a subject, the method comprising administering to the subject any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein, to thereby increase the level of fetal hemoglobin the subject. In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to increase the level of fetal hemoglobin the subject.

In one aspect, provided herein are methods of inducing expression of fetal hemoglobin in a subject, the method comprising administering to the subject any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein, to thereby induce expression of fetal hemoglobin the subject. In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to induce expression of fetal hemoglobin the subject.

In one aspect, provided herein are methods of increasing the ratio of fetal hemoglobin to adult hemoglobin in a subject, the method comprising administering to the subject any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein, to thereby increase the ratio of fetal hemoglobin to adult hemoglobin in the subject. In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to increase the ratio of fetal hemoglobin to adult hemoglobin in the subject.

Standard assays to measure the level of fetal hemoglobin are known in the art. For example, immunoassays such as a western blot and an enzyme-linked immunosorbent assay (ELISA) can be utilized to evaluate protein levels. mRNA levels can be measured by, e.g., a northern blot or by RT-qPCR, a nuclease protection assay, or in situ hybridization. Fetal hemoglobin can be measured in, e.g., sample from a subject (e.g., a blood or tissue sample).

### 4.13.4 Methods of Treating, Ameliorating, or Preventing a BCL11A Associated Disease

In one aspect, provided herein are methods of treating, ameliorating, or preventing a disease in a subject, the method comprising administering to the subject any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein, to thereby treat, ameliorate, or prevent a disease in the subject. In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the disease in the subject.

In one aspect, provided herein are methods of treating, ameliorating, or preventing a BCL11A (e.g., hBCL11A) associated disease in a subject, the method comprising administering to the subject any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein, to thereby treat, ameliorate, or prevent the BCL11A (e.g., hBCL11A) associated disease in the subject. In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the BCL11A (e.g., hBCL11A) associated disease in the subject.

In some embodiments, the BCL11A associated disease is an inherited blood disorder. In some embodiments, the inherited blood disorder is a hemoglobinopathy (e.g., a hemoglobinopathy described herein). In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, or methemoglobinemia.

In some embodiments, the inherited blood disorder is an inherited bone marrow failure syndrome (e.g., an inherited bone marrow failure syndrome described herein). In some embodiments, inherited bone marrow failure syndrome is amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, thrombocytopenia absent radii (TAR), a SAMD9/SAMD9L disorder, or a MECOM-associated syndrome.

In one aspect, provided herein are dsRNA agents, conjugates, vectors, carriers, and pharmaceutical compositions for use in a method of treating, ameliorating, or preventing a BCL11A (e.g., hBCL11A) associated disease in a subject, the method comprising administering to the subject a dsRNA agent described herein, a conjugate described herein, a vector described herein, a carrier described herein, or a pharmaceutical composition described herein, to thereby treat, ameliorate, or prevent the BCL11A (e.g., hBCL11A) associated disease in the subject.

In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the BCL11A (e.g., hBCL11A) associated disease in the subject.

In some embodiments, the BCL11A associated disease is an inherited blood disorder. In some embodiments, the inherited blood disorder is a hemoglobinopathy (e.g., a hemoglobinopathy described herein). In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, or methemoglobinemia.

In some embodiments, the BCL11A associated disease is an inherited bone marrow failure syndrome. In some embodiments, the inherited bone marrow failure syndrome is amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, thrombocytopenia absent radii (TAR), a SAMD9/SAMD9L disorder, or a MECOM-associated syndrome.

In one aspect, provided herein are uses of dsRNA agents, conjugates, vectors, carriers, and pharmaceutical compositions in the manufacture of a medicament for the treatment, amelioration, or prevention of a BCL11A (e.g., hBCL11A) associated disease in a subject.

In some embodiments, the agent (e.g., RNAi agent, dsRNA agent, sense strand, antisense strand, ASO, ssRNA agent, etc.), conjugate, vector, carrier, or pharmaceutical composition is administered to the sub eject in an amount and for a time sufficient to treat, ameliorate, or prevent the BCL11A (e.g., hBCL11A) associated disease in the subject.

In some embodiments, the BCL11A associated disease is an inherited blood disorder. In some embodiments, the inherited blood disorder is a hemoglobinopathy (e.g., a hemoglobinopathy described herein). In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, or methemoglobinemia.

In some embodiments, the inherited blood disorder is an inherited bone marrow failure syndrome (e.g., an inherited bone marrow failure syndrome described herein). In some embodiments, inherited bone marrow failure syndrome is amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, thrombocytopenia absent radii (TAR), a SAMD9/SAMD9L disorder, or a MECOM-associated syndrome.

In one aspect, provided herein are uses of dsRNA agents, conjugates, vectors, carriers, and pharmaceutical compositions in the manufacture of a medicament for use in a method of treating, ameliorating, or preventing a BCL11A (e.g., hBCL11A) associated disease in a subject, the method comprising administering to the subject a dsRNA agent described herein, a conjugate described herein, a vector described herein, a carrier described herein, or a pharmaceutical composition described herein, to thereby treat, ameliorate, or prevent the BCL11A (e.g., hBCL11A) associated disease in the subject.

In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the BCL11A (e.g., hBCL11A) associated disease in the subject.

In some embodiments, the BCL11A associated disease is an inherited blood disorder. In some embodiments, the inherited blood disorder is a hemoglobinopathy (e.g., a hemoglobinopathy described herein. In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, or methemoglobinemia.

In some embodiments, the inherited blood disorder is an inherited bone marrow failure syndrome (e.g., an inherited bone marrow failure syndrome described herein. In some embodiments, inherited bone marrow failure syndrome is amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, thrombocytopenia absent radii (TAR), a SAMD9/SAMD9L disorder, or a MECOM-associated syndrome.

In one aspect, provided herein are dsRNA agents, conjugates, vectors, carriers, and pharmaceutical compositions for use in treating a disease in a subject, preferably a BCL11A (e.g., hBCL11A) associated disease, more preferably a liver disease.

In one aspect, provided herein are dsRNA agents, conjugates, vectors, carriers, and pharmaceutical compositions for use for use in treating a disease in a subject by reducing or inhibiting expression of BCL11A (e.g., hBCL11A) in a cell.

### 4.13.5 Methods of Treating, Ameliorating, or Preventing an Inherited Blood Disorder

In one aspect, provided herein are methods of treating, ameliorate, or preventing an inherited blood disorder in a subject, the method comprising administering to the subject any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein, to thereby treat, ameliorate, or prevent the inherited blood disorder in the subject. In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the inherited blood disorder in the subject.

In some embodiments, the inherited blood disorder is a hemoglobinopathy (e.g., a hemoglobinopathy described herein). In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, or methemoglobinemia.

In some embodiments, the inherited blood disorder is an inherited bone marrow failure syndrome (e.g., an inherited bone marrow failure syndrome described herein). In some embodiments, inherited bone marrow failure syndrome is amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, thrombocytopenia absent radii (TAR), a SAMD9/SAMD9L disorder, or a MECOM-associated syndrome.

In one aspect, provided herein are dsRNA agents, conjugates, vectors, carriers, and pharmaceutical compositions for use in a method of treating, ameliorating, or preventing an inherited blood disorder in a subject, the method comprising administering to the subject a dsRNA agent described herein, a conjugate described herein, a vector described herein, a carrier described herein, or a pharmaceutical composition described herein, to thereby treat, ameliorate, or prevent the inherited blood disorder in the subject.

In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the inherited blood disorder in the subject.

In some embodiments, the inherited blood disorder is a hemoglobinopathy (e.g., a hemoglobinopathy described herein). In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, or methemoglobinemia.

In some embodiments, the inherited blood disorder is an inherited bone marrow failure syndrome (e.g., an inherited bone marrow failure syndrome described herein). In some embodiments, inherited bone marrow failure syndrome is amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, thrombocytopenia absent radii (TAR), a SAMD9/SAMD9L disorder, or a MECOM-associated syndrome.

In one aspect, provided herein are uses of dsRNA agents, conjugates, vectors, carriers, and pharmaceutical compositions in the manufacture of a medicament for the treatment, amelioration, or prevention of an inherited blood disorder in a subject.

In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the inherited blood disorder in the subject.

In some embodiments, the inherited blood disorder is a hemoglobinopathy (e.g., a hemoglobinopathy described herein). In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, or methemoglobinemia.

In some embodiments, the inherited blood disorder is an inherited bone marrow failure syndrome (e.g., an inherited bone marrow failure syndrome described herein). In some embodiments, inherited bone marrow failure syndrome is amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, thrombocytopenia absent radii (TAR), a SAMD9/SAMD9L disorder, or a MECOM-associated syndrome.

In one aspect, provided herein are uses of dsRNA agents, conjugates, vectors, carriers, and pharmaceutical compositions in the manufacture of a medicament for use in a method of treating, ameliorating, or preventing an inherited blood disorder in a subject, the method comprising administering to the subject a dsRNA agent described herein, a conjugate described herein, a vector described herein, a carrier described herein, or a pharmaceutical composition described herein, to thereby treat, ameliorate, or prevent the inherited blood disorder in the subject.

In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the inherited blood disorder in the subject.

In some embodiments, the inherited blood disorder is a hemoglobinopathy (e.g., a hemoglobinopathy described herein). In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, or methemoglobinemia.

In some embodiments, the inherited blood disorder is an inherited bone marrow failure syndrome (e.g., an inherited bone marrow failure syndrome described herein). In some embodiments, inherited bone marrow failure syndrome is amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, thrombocytopenia absent radii (TAR), a SAMD9/SAMD9L disorder, or a MECOM-associated syndrome.

### 4.13.6 Methods of Treating, Ameliorating, or Preventing a Hemoglobinopathy

In one aspect, provided herein are methods of treating, ameliorate, or preventing an hemoglobinopathy in a subject, the method comprising administering to the subject any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein, to thereby treat, ameliorate, or prevent the hemoglobinopathy in the subject. In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the hemoglobinopathy in the subject.

In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, methemoglobinemia, or any combination thereof. In some embodiments, the hemoglobinopathy is sickle cell disease or a thalassemia (e.g., α-thalassemia, β-thalassemia, δ-thalassemia, or γ-thalassemia). In some embodiments, the hemoglobinopathy is sickle cell disease. In some embodiments, the hemoglobinopathy is β-thalassemia.

In some embodiments, the subject is suspected of having or has been diagnosed with sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, methemoglobinemia, or any combination thereof. In some embodiments, the subject is suspected of having or has been diagnosed with sickle cell disease or a thalassemia (e.g., α-thalassemia, β-thalassemia, δ-thalassemia, or γ-thalassemia).

In one aspect, provided herein are dsRNA agents, conjugates, vectors, carriers, and pharmaceutical compositions for use in a method of treating, ameliorating, or preventing an hemoglobinopathy in a subject, the method comprising administering to the subject a dsRNA agent described herein, a conjugate described herein, a vector described herein, a carrier described herein, or a pharmaceutical composition described herein, to thereby treat, ameliorate, or prevent the hemoglobinopathy in the subject.

In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the hemoglobinopathy in the subject.

In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, methemoglobinemia, or any combination thereof. In some embodiments, the hemoglobinopathy is sickle cell disease or a thalassemia (e.g., α-thalassemia, β-thalassemia, δ-thalassemia, or γ-thalassemia). In some embodiments, the hemoglobinopathy is sickle cell disease. In some embodiments, the hemoglobinopathy is β-thalassemia.

In some embodiments, the subject is suspected of having or has been diagnosed with sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, methemoglobinemia, or any combination thereof. In some embodiments, the subject is suspected of having or has been diagnosed with sickle cell disease or a thalassemia (e.g., α-thalassemia, β-thalassemia, δ-thalassemia, or γ-thalassemia).

In one aspect, provided herein are uses of dsRNA agents, conjugates, vectors, carriers, and pharmaceutical compositions in the manufacture of a medicament for the treatment, amelioration, or prevention of an hemoglobinopathy in a subject.

In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the hemoglobinopathy in the subject.

In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, methemoglobinemia, or any combination thereof. In some embodiments, the hemoglobinopathy is sickle cell disease or a thalassemia (e.g., α-thalassemia, β-thalassemia, δ-thalassemia, or γ-thalassemia). In some embodiments, the hemoglobinopathy is sickle cell disease. In some embodiments, the hemoglobinopathy is β-thalassemia.

In some embodiments, the subject is suspected of having or has been diagnosed with sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, methemoglobinemia, or any combination thereof. In some embodiments, the subject is suspected of having or has been diagnosed with sickle cell disease or a thalassemia (e.g., α-thalassemia, β-thalassemia, δ-thalassemia, or γ-thalassemia).

In one aspect, provided herein are uses of dsRNA agents, conjugates, vectors, carriers, and pharmaceutical compositions in the manufacture of a medicament for use in a method of treating, ameliorating, or preventing an hemoglobinopathy in a subject, the method comprising administering to the subject a dsRNA agent described herein, a conjugate described herein, a vector described herein, a carrier described herein, or a pharmaceutical composition described herein, to thereby treat, ameliorate, or prevent the hemoglobinopathy in the subject.

In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the hemoglobinopathy in the subject.

In some embodiments, the hemoglobinopathy is sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, methemoglobinemia, or any combination thereof. In some embodiments, the hemoglobinopathy is sickle cell disease or a thalassemia (e.g., α-thalassemia, β-thalassemia, δ-thalassemia, or γ-thalassemia). In some embodiments, the hemoglobinopathy is sickle cell disease. In some embodiments, the hemoglobinopathy is β-thalassemia.

In some embodiments, the subject is suspected of having or has been diagnosed with sickle cell disease, sickle cell trait, hemoglobin C disease, hemoglobin C trait, hemoglobin S/C disease, hemoglobin D disease, hemoglobin E disease, a thalassemia (e.g., α-thalassemia, β-thalassemia, δ-thalassemia, or γ-thalassemia), a condition associated with hemoglobin with increased oxygen affinity, a condition associated with hemoglobin with decreased oxygen affinity, unstable hemoglobin disease, methemoglobinemia, or any combination thereof. In some embodiments, the subject is suspected of having or has been diagnosed with sickle cell disease or a thalassemia (e.g., α-thalassemia, β-thalassemia, δ-thalassemia, or γ-thalassemia).

### 4.13.7 Methods of Treating, Ameliorating, or Preventing an Inherited Bone Marrow Failure Syndrome

In one aspect, provided herein are methods of treating, ameliorate, or preventing an inherited bone marrow failure syndrome in a subject, the method comprising administering to the subject any one more agent described herein (e.g., an RNAi agent, a dsRNA agent, a sense strand, an antisense strand, an ASO, a ssRNA agent, etc.); a vector described herein; a conjugate described herein; a carrier described herein; a host cell described herein; and/or a pharmaceutical composition described herein, to thereby treat, ameliorate, or prevent the inherited bone marrow failure syndrome in the subject. In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the inherited bone marrow failure syndrome in the subject.

In some embodiments, the inherited bone marrow failure syndrome is amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, thrombocytopenia absent radii (TAR), a SAMD9/SAMD9L disorder, or a MECOM-associated syndrome.

In some embodiments, the subject is suspected of having or has been diagnosed with amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, or thrombocytopenia absent radii (TAR).

In one aspect, provided herein are dsRNA agents, conjugates, vectors, carriers, and pharmaceutical compositions for use in a method of treating, ameliorating, or preventing an inherited bone marrow failure syndrome in a subject, the method comprising administering to the subject a dsRNA agent described herein, a conjugate described herein, a vector described herein, a carrier described herein, or a pharmaceutical composition described herein, to thereby treat, ameliorate, or prevent the inherited bone marrow failure syndrome in the subject.

In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the inherited bone marrow failure syndrome in the subject.

In some embodiments, the inherited bone marrow failure syndrome is amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, thrombocytopenia absent radii (TAR), a SAMD9/SAMD9L disorder, or a MECOM-associated syndrome.

In some embodiments, the subject is suspected of having or has been diagnosed with amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, or thrombocytopenia absent radii (TAR).

In one aspect, provided herein are uses of dsRNA agents, conjugates, vectors, carriers, and pharmaceutical compositions in the manufacture of a medicament for the treatment, amelioration, or prevention of an inherited bone marrow failure syndrome in a subject.

In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the inherited bone marrow failure syndrome in the subject.

In some embodiments, the inherited bone marrow failure syndrome is amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, thrombocytopenia absent radii (TAR), a SAMD9/SAMD9L disorder, or a MECOM-associated syndrome.

In some embodiments, the subject is suspected of having or has been diagnosed with amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, or thrombocytopenia absent radii (TAR).

In one aspect, provided herein are uses of dsRNA agents, conjugates, vectors, carriers, and pharmaceutical compositions in the manufacture of a medicament for use in a method of treating, ameliorating, or preventing an inherited bone marrow failure syndrome in a subject, the method comprising administering to the subject a dsRNA agent described herein, a conjugate described herein, a vector described herein, a carrier described herein, or a pharmaceutical composition described herein, to thereby treat, ameliorate, or prevent the inherited bone marrow failure syndrome in the subject.

In some embodiments, the agent (e.g., RNAi agent, dsRNA agent, sense strand, antisense strand, ASO, ssRNA agent, etc.), conjugate, vector, carrier, or pharmaceutical composition is administered to the subject in an amount and for a time sufficient to treat, ameliorate, or prevent the inherited bone marrow failure syndrome in the subject.

In some embodiments, the inherited bone marrow failure syndrome is amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, thrombocytopenia absent radii (TAR), a SAMD9/SAMD9L disorder, or a MECOM-associated syndrome.

In some embodiments, the subject is suspected of having or has been diagnosed with amegakaryocytic thrombocytopenia (Amega), diamond blackfan anemia (DBA), dyskeratosis congenita (DC), Fanconi anemia (FA), Pearson syndrome, severe congenital neutropenia (SCN), Schwachman diamond syndrome (SDS), GATA2 deficiency, cyclic neutropenia, Dubowitz syndrome, Kostmann syndrome, refractory cytopenia, or thrombocytopenia absent radii (TAR).

### 4.14 Kits

In a one aspect, provided herein are kits comprising any one or more agent described herein (e.g., an RNAi agent, a dsRNA agent, a sense strand, an antisense strand, an ASO, a ssRNA agent, etc.) (see, e.g., §§ 4.2, 4.3, 4.4); a vector described herein (see, e.g., § 4.9); a conjugate described herein (see, e.g., § 4.5); a carrier described herein (see, e.g., § 4.10); a host cell described herein (see, e.g., § 4.11); and/or a pharmaceutical composition described herein (see, e.g., § 4.12); or any combination thereof. In addition, the kit may comprise a liquid vehicle for solubilizing or diluting, and/or technical instructions. The technical instructions of the kit may contain information about administration and dosage and subject groups.

In some embodiments, the agent (e.g., described herein) (e.g., RNAi agent, dsRNA agent, sense strand, antisense strand, ASO, ssRNA agent, etc.), vector (e.g., described herein), conjugate (e.g., described herein), carrier (e.g., described herein), host cell (e.g., described herein), and/or pharmaceutical composition (e.g., described herein) is provided in a separate part of the kit. In some embodiments, the agent (e.g., described herein) (e.g., RNAi agent, dsRNA agent, sense strand, antisense strand, ASO, ssRNA agent, etc.), vector (e.g., described herein), conjugate (e.g., described herein), carrier (e.g., described herein), host cell (e.g., described herein), and/or pharmaceutical composition (e.g., described herein) is optionally lyophilized, spray-dried, or spray-freeze dried. The kit may further contain as a part a vehicle (e.g., buffer solution) for solubilizing the dried or lyophilized any agent (e.g., described herein) (e.g., RNAi agent, dsRNA agent, sense strand, antisense strand, ASO, ssRNA agent, etc.), vector (e.g., described herein), conjugate (e.g., described herein), carrier (e.g., described herein), host cell (e.g., described herein), and/or pharmaceutical composition (e.g., described herein).

In some embodiments, the kit comprises a single dose container. In some embodiments, the kit comprises a multi-dose container. In some embodiments, the kit comprises an administration device (e.g., an injector for intradermal injection or a syringe for intramuscular injection).

Any of the kits described herein may be used in any of the methods described herein (see, e.g., § 4.13).

### 5. EXAMPLES

TABLE OF CONTENTS

| | |
|---|---|
| 5.1 | Example 1. Design of BCL11A Targeting RNA Agents. |
| 5.2 | Example 2. Synthesis of BCL11A Targeting RNA Agents. |
| 5.3 | Example 3. In Vitro RNA Agent Mediated Knockdown of hBCL11A in HEK293 |
| | Cells. |
| 5.5 | Example 4. In Vitro RNA Agent Mediated Knockdown of hBCL11A in HUDEP2 |
| | Cells. |

### 5.5 Example 5. In Vitro RNA Agent Mediated Knockdown of hBCL11A in HEK293 Cells

### 5.1 Example 1. Design of BCL11A Targeting RNA Agents

The inventors, inter alia, designed RNA agents (including dsRNA agents and ASO agents) targeting BCL11A. The nucleotide sequence of the unmodified BCL11A sense and antisense strands of each of the dsRNA agents are set forth in Tables 2-3; and the nucleotide sequence of the exemplary modified BCL11A sense and antisense strands of each of the dsRNA agents are set forth in Tables 4-5. The inventors, inter alia, designed ASO agents targeting BCL11A. The nucleotide sequence of the unmodified and modified ASOs is set forth in Tables 7 and Table 9.

### 5.2 Example 2. Synthesis of BCL11A Targeting RNA Agents

The RNA agents designed in Example 1 were synthesized using standard methods known in the art and described herein. Briefly, the RNA agents were synthesized using a Mermade 192 synthesizer (BioAutomation) on controlled pore glass (500-1000 A) solid supports loaded with a first nucleotide of interest. Upon completion of the solid phase synthesis, solid-supported polynucleotides were treated with Methylamine (40% aqueous) at room temperature in 96 well plates for approximately 2 hours to afford cleavage from the solid support and subsequent removal of relevant protecting groups. Polynucleotides were precipitated by the addition of 1 mL of 9:1 acetontrile:ethanol or 1:1 ethanol:isopropanol. The plates were then centrifuged at 4° C. for 45 minutes and the supernatant removed. The polynucleotide pellet was resuspended in 20 mM NaOAc and subsequently desalted using a HiTrap size exclusion column (5 mL, GE Healthcare) on an Agilent LC system equipped with an autosampler, UV detector, conductivity meter, and fraction collector. Desalted samples were collected in 96 well plates and then analyzed by LC-MS and UV spectrometry to confirm identity and quantify the amount of material, respectively.

Duplexing of single strands for dsRNA agents was performed on a Tecan liquid handling robot. Sense and antisense single strands were combined in an equimolar ratio to a final concentration of 10 μM in lx PBS in 96 well plates, the plate was sealed, incubated at 100° C. for 10 minutes, and was subsequently allowed to return slowly to room temperature over a period of 2-3 hours. The concentration and identity of each duplex was confirmed and was then subsequently utilized for screening assays.

### 5.3 Example 3. In Vitro RNA Agent Mediated Knockdown of hBCL11A in HEK293 Cells

Each of the indicated dsRNA Agents from Table 3 (modified agents) and the ASO Agents indicated from Table 9 (modified agents) above were evaluated for their ability to knockdown hBCL11A expression in vitro in HEK293 cells.

Briefly, HEK293 cells were seeded at 2.0e5 cells per well in a standard 96 well plate. The cells were transfected with the indicated hBCL11A targeting dsRNA agent (2 nM and 0.2 nM) using Opti-MEM/LF2000 mix according to the manufacturer's instructions and incubated for 24 hours. The level of hBCL11A was assessed utilizing a standard bDNA assay according to the manufacturer's instructions. The expression of hBCL11A was normalized to the expression of human GAPDH. Each treatment group was run in quadruplicate, the mean and standard deviation calculated.

Table 19 shows the percent of maximum inhibition of hBCL11A mRNA after treatment with the indicated RNA agent (normalized to GAPDH). “MV” in Table 19 indicates the mean value; and “SD” indicates the standard deviation, with

TABLE 19
Maximum Inhibition of hBCL11A mRNA (Normalized to GAPDH).

| | 2 nM | | | | 0.2 nM | | | |
|---|---|---|---|---|---|---|---|---|
| RNA | MV | SD | | | MV | SD | | |
| Agent | hBCL11a/ | hBCL11a/ | MV | SD | hBCL11a/ | hsBCL11a/ | MV | SD |
| ID | hGAPDH | hGAPDH | hGAPDH | hGAPDH | hGAPDH | hGAPDH | hGAPDH | hGAPDH |
| dsRNA 1399 | 85.5 | 1.8 | 71.2 | 9.8 | 88.1 | 2.3 | 79.5 | 10.1 |
| dsRNA 1400 | 81.0 | 6.9 | 57.6 | 1.8 | 92.9 | 8.1 | 81.3 | 12.8 |
| dsRNA 1401 | 35.2 | 3.1 | 83.0 | 7.0 | 38.0 | 6.0 | 100.9 | 6.6 |
| dsRNA 1402 | 33.2 | 3.1 | 80.4 | 9.7 | 32.3 | 4.0 | 92.8 | 8.5 |
| dsRNA 772 | 29.5 | 5.1 | 76.5 | 9.8 | 30.8 | 3.4 | 80.9 | 10.0 |
| dsRNA 1403 | 40.5 | 6.2 | 74.1 | 3.0 | 40.5 | 4.4 | 78.7 | 7.4 |
| dsRNA 1403 | 38.1 | 4.7 | 57.5 | 1.2 | 43.9 | 3.4 | 78.0 | 10.8 |
| dsRNA 780 | 32.2 | 5.0 | 79.0 | 3.2 | 36.6 | 3.8 | 68.5 | 6.1 |
| dsRNA 780 | 33.8 | 3.6 | 59.4 | 2.2 | 38.7 | 3.2 | 75.1 | 9.8 |
| dsRNA 1404 | 43.6 | 4.7 | 73.4 | 2.1 | 57.2 | 7.3 | 71.4 | 13.2 |
| dsRNA 1404 | 48.9 | 5.6 | 47.9 | 1.8 | 57.7 | 5.4 | 67.4 | 5.0 |
| dsRNA 1405 | 39.1 | 5.6 | 53.8 | 2.3 | 46.5 | 6.2 | 62.6 | 6.5 |
| dsRNA 1406 | 34.1 | 2.9 | 82.3 | 4.7 | 40.1 | 6.4 | 79.4 | 13.2 |
| dsRNA 1406 | 32.5 | 2.2 | 56.7 | 1.5 | 39.9 | 3.9 | 72.8 | 5.8 |
| dsRNA 1407 | 66.7 | 6.3 | 74.1 | 4.3 | 71.2 | 6.8 | 80.1 | 11.2 |
| dsRNA 1408 | 49.5 | 2.3 | 73.0 | 3.5 | 67.9 | 7.3 | 77.2 | 3.9 |
| ASO 60 | 18.3 | 2.5 | 84.3 | 6.0 | 55.8 | 4.2 | 79.8 | 4.2 |
| dsRNA 1409 | 45.6 | 1.6 | 85.8 | 7.1 | 47.6 | 6.9 | 98.8 | 4.7 |
| ASO 61 | 19.5 | 2.1 | 96.3 | 6.0 | 28.9 | 2.2 | 94.1 | 5.2 |
| dsRNA 1410 | 68.5 | 5.1 | 77.1 | 6.5 | 69.3 | 12.0 | 94.6 | 5.2 |
| dsRNA 1410 | 71.0 | 4.0 | 57.7 | 3.8 | 81.7 | 4.9 | 75.6 | 10.2 |
| ASO 62 | 14.2 | 1.0 | 75.0 | 5.5 | 26.4 | 4.1 | 81.0 | 1.9 |
| dsRNA 1411 | 26.8 | 1.5 | 78.6 | 6.1 | 29.3 | 4.1 | 76.6 | 5.8 |
| ASO 63 | 12.6 | 1.5 | 53.4 | 2.5 | 24.3 | 4.1 | 76.4 | 11.0 |
| dsRNA 1412 | 26.5 | 2.3 | 77.2 | 3.8 | 31.7 | 2.6 | 73.3 | 3.1 |
| dsRNA 1413 | 40.9 | 4.5 | 86.0 | 4.5 | 45.0 | 5.6 | 74.8 | 8.6 |
| dsRNA 1414 | 67.7 | 2.9 | 65.3 | 1.7 | 60.2 | 4.5 | 74.6 | 8.1 |
| dsRNA 1415 | 35.1 | 5.1 | 59.0 | 2.1 | 37.5 | 2.7 | 70.9 | 8.0 |
| dsRNA 1416 | 62.9 | 6.5 | 70.1 | 2.4 | 69.0 | 4.5 | 81.9 | 14.1 |
| dsRNA 1417 | 71.2 | 4.0 | 84.9 | 5.5 | 79.4 | 6.7 | 99.2 | 7.0 |
| dsRNA 1418 | 49.8 | 5.2 | 75.2 | 8.1 | 65.5 | 7.1 | 87.6 | 8.5 |
| dsRNA 1419 | 46.3 | 5.4 | 46.4 | 2.8 | 76.6 | 8.4 | 86.3 | 13.7 |
| dsRNA 1420 | 53.9 | 2.7 | 46.4 | 2.7 | 77.2 | 8.9 | 81.1 | 9.9 |
| dsRNA 1421 | 43.9 | 3.7 | 60.7 | 4.9 | 53.0 | 4.8 | 80.8 | 12.1 |
| dsRNA 782 | 34.4 | 1.9 | 90.3 | 3.4 | 48.8 | 4.2 | 78.2 | 3.9 |
| dsRNA 1422 | 62.2 | 2.1 | 86.4 | 3.6 | 74.4 | 3.3 | 71.5 | 8.2 |
| dsRNA 1423 | 45.9 | 5.3 | 70.4 | 1.2 | 51.0 | 1.4 | 70.0 | 8.8 |
| dsRNA 1424 | 51.0 | 3.3 | 71.7 | 5.5 | 51.6 | 0.6 | 70.9 | 11.3 |
| dsRNA 1425 | 43.2 | 3.4 | 74.6 | 6.0 | 49.8 | 4.7 | 75.0 | 9.8 |
| dsRNA 1426 | 49.2 | 3.1 | 75.4 | 3.0 | 54.9 | 4.5 | 82.2 | 5.9 |
| dsRNA 1427 | 33.7 | 2.1 | 97.8 | 2.2 | 40.9 | 7.6 | 100.5 | 4.7 |
| dsRNA 1428 | 48.3 | 3.3 | 98.2 | 6.4 | 51.6 | 7.4 | 91.2 | 1.1 |
| dsRNA 1429 | 35.5 | 3.1 | 99.3 | 11.4 | 37.5 | 6.7 | 85.7 | 6.0 |
| dsRNA 1430 | 27.9 | 2.7 | 101.3 | 6.4 | 33.0 | 5.3 | 79.1 | 6.1 |
| dsRNA 1431 | 30.0 | 3.3 | 105.8 | 8.2 | 41.5 | 7.5 | 70.2 | 4.7 |
| dsRNA 1432 | 38.6 | 5.4 | 99.9 | 7.6 | 52.8 | 6.0 | 64.7 | 7.9 |
| dsRNA 1433 | 30.9 | 4.3 | 96.5 | 8.2 | 39.4 | 5.3 | 68.1 | 7.1 |
| dsRNA 1434 | 36.4 | 2.1 | 95.3 | 7.4 | 40.2 | 5.0 | 69.3 | 5.7 |
| dsRNA 1435 | 42.3 | 2.5 | 83.9 | 2.1 | 46.9 | 5.9 | 80.3 | 9.5 |
| dsRNA 1436 | 40.5 | 2.2 | 89.6 | 6.6 | 39.4 | 1.3 | 88.9 | 3.3 |
| dsRNA 1437 | 51.3 | 2.0 | 86.9 | 4.0 | 58.6 | 5.9 | 82.3 | 3.3 |
| dsRNA 1438 | 46.5 | 3.6 | 81.8 | 2.1 | 44.5 | 5.5 | 76.4 | 5.2 |
| dsRNA 1439 | 48.2 | 4.1 | 76.7 | 1.5 | 48.6 | 7.5 | 73.6 | 4.7 |
| dsRNA 1440 | 43.8 | 4.9 | 80.7 | 3.8 | 50.9 | 6.6 | 69.5 | 5.2 |
| dsRNA 1441 | 35.1 | 4.2 | 88.9 | 4.3 | 40.6 | 3.5 | 70.1 | 9.9 |
| dsRNA 777 | 33.3 | 4.5 | 85.6 | 2.0 | 35.0 | 1.9 | 70.0 | 11.1 |
| dsRNA 1442 | 33.0 | 3.4 | 86.3 | 4.5 | 37.9 | 2.6 | 68.9 | 7.7 |
| dsRNA 1443 | 33.1 | 2.5 | 81.0 | 6.7 | 39.3 | 2.4 | 65.7 | 4.8 |
| dsRNA 1444 | 33.8 | 0.5 | 81.6 | 4.3 | 40.0 | 1.0 | 74.9 | 10.2 |
| dsRNA 1445 | 41.0 | 4.2 | 83.9 | 0.9 | 45.5 | 0.7 | 91.0 | 10.9 |
| dsRNA 1446 | 38.4 | 1.9 | 105.3 | 4.5 | 39.5 | 4.8 | 101.8 | 8.4 |
| dsRNA 1447 | 51.7 | 6.5 | 94.7 | 4.3 | 39.5 | 3.3 | 95.7 | 9.9 |
| dsRNA 1448 | 37.4 | 5.0 | 90.7 | 7.7 | 31.4 | 2.1 | 98.2 | 8.0 |
| dsRNA 779 | 38.5 | 3.5 | 75.5 | 9.1 | 34.7 | 5.0 | 84.6 | 8.0 |
| dsRNA 1449 | 83.8 | 5.3 | 86.3 | 11.0 | 84.2 | 5.0 | 67.7 | 6.1 |
| dsRNA 1450 | 81.6 | 6.2 | 82.8 | 12.6 | 88.7 | 7.8 | 61.3 | 6.1 |
| dsRNA 1451 | 64.6 | 8.7 | 89.8 | 4.7 | 75.9 | 9.9 | 64.4 | 3.8 |
| dsRNA 1452 | 64.7 | 8.2 | 93.5 | 4.0 | 64.7 | 4.5 | 61.4 | 3.0 |
| dsRNA 1453 | 49.2 | 2.1 | 56.1 | 9.0 | 59.5 | 6.3 | 80.6 | 12.0 |
| dsRNA 1454 | 56.8 | 8.0 | 86.0 | 3.3 | 65.0 | 13.0 | 64.9 | 4.2 |
| dsRNA 1455 | 69.6 | 13.7 | 83.7 | 3.7 | 78.1 | 8.2 | 61.7 | 1.6 |
| dsRNA 1456 | 72.4 | 1.9 | 66.0 | 11.7 | 76.8 | 5.8 | 80.9 | 8.8 |
| dsRNA 1457 | 51.0 | 5.2 | 82.1 | 5.7 | 54.4 | 4.8 | 66.9 | 6.7 |
| dsRNA 1458 | 44.8 | 2.7 | 73.4 | 6.9 | 55.7 | 2.0 | 84.6 | 7.0 |
| dsRNA 1459 | 57.1 | 3.3 | 68.3 | 6.9 | 58.1 | 4.7 | 93.2 | 8.5 |
| dsRNA 1460 | 83.6 | 6.8 | 85.1 | 8.6 | 83.7 | 4.7 | 63.2 | 2.0 |
| dsRNA 1461 | 56.4 | 1.3 | 94.7 | 3.5 | 69.3 | 8.1 | 78.8 | 7.3 |
| dsRNA 1462 | 48.6 | 3.2 | 61.3 | 3.1 | 57.5 | 5.3 | 88.4 | 14.5 |
| dsRNA 1463 | 53.0 | 3.4 | 86.2 | 6.2 | 61.0 | 7.2 | 80.2 | 4.9 |
| dsRNA 1464 | 46.4 | 3.9 | 74.4 | 4.0 | 55.8 | 3.3 | 69.7 | 6.9 |
| dsRNA 1465 | 64.8 | 1.3 | 96.6 | 11.8 | 59.7 | 3.4 | 90.5 | 7.1 |
| dsRNA 1466 | 58.8 | 4.1 | 71.6 | 1.5 | 68.0 | 8.9 | 64.7 | 6.7 |
| dsRNA 1467 | 50.2 | 4.1 | 79.2 | 3.1 | 68.6 | 8.8 | 65.7 | 5.8 |
| dsRNA 1468 | 43.3 | 3.5 | 61.4 | 2.4 | 56.7 | 7.3 | 82.1 | 10.6 |
| dsRNA 1469 | 73.0 | 11.0 | 85.3 | 6.0 | 79.1 | 4.6 | 69.2 | 5.9 |
| dsRNA 1470 | 67.6 | 3.0 | 76.1 | 3.8 | 78.3 | 9.9 | 77.3 | 13.0 |
| dsRNA 1471 | 50.8 | 4.6 | 78.9 | 1.4 | 51.0 | 7.3 | 77.4 | 4.4 |
| dsRNA 1472 | 52.1 | 0.8 | 74.5 | 6.7 | 60.7 | 6.7 | 73.6 | 4.0 |
| dsRNA 1473 | 84.1 | 5.4 | 83.5 | 8.5 | 80.9 | 7.9 | 81.6 | 8.2 |
| dsRNA 1474 | 83.2 | 4.1 | 94.5 | 8.9 | 87.2 | 6.2 | 92.8 | 13.1 |
| dsRNA 1475 | 77.5 | 4.7 | 78.2 | 4.6 | 77.1 | 5.8 | 86.8 | 10.9 |
| dsRNA 1476 | 76.3 | 5.2 | 69.5 | 4.6 | 85.0 | 7.5 | 68.8 | 6.1 |
| dsRNA 1477 | 70.5 | 7.1 | 68.3 | 11.4 | 70.2 | 8.5 | 73.1 | 8.0 |
| dsRNA 1478 | 46.6 | 3.5 | 80.1 | 9.9 | 54.2 | 8.0 | 75.4 | 10.6 |
| dsRNA 783 | 36.2 | 3.7 | 80.1 | 4.3 | 48.2 | 6.7 | 78.9 | 3.1 |
| dsRNA 1479 | 57.7 | 6.4 | 77.0 | 6.6 | 72.0 | 13.4 | 79.9 | 3.8 |
| dsRNA 1480 | 45.6 | 3.6 | 54.4 | 3.6 | 55.3 | 6.5 | 66.4 | 9.4 |
| dsRNA 1481 | 39.3 | 4.5 | 73.9 | 7.8 | 53.2 | 7.9 | 82.0 | 2.9 |
| dsRNA 1482 | 70.0 | 7.0 | 73.3 | 4.6 | 70.3 | 7.2 | 86.3 | 7.2 |
| dsRNA 1483 | 69.4 | 4.8 | 72.6 | 5.0 | 77.8 | 7.7 | 91.7 | 7.9 |
| dsRNA 1484 | 64.3 | 4.0 | 78.5 | 5.9 | 82.7 | 4.3 | 91.2 | 3.7 |
| dsRNA 1485 | 92.2 | 3.1 | 80.2 | 8.8 | 92.0 | 7.8 | 80.1 | 9.0 |
| dsRNA 1486 | 83.1 | 4.1 | 70.0 | 6.4 | 84.1 | 6.2 | 73.5 | 11.6 |
| dsRNA 1487 | 87.0 | 4.7 | 66.6 | 5.6 | 89.4 | 11.0 | 63.6 | 1.1 |
| dsRNA 1488 | 74.1 | 6.0 | 64.1 | 5.6 | 79.7 | 10.1 | 63.2 | 5.1 |
| dsRNA 1489 | 42.4 | 1.7 | 83.5 | 7.6 | 53.8 | 4.8 | 76.0 | 6.6 |
| dsRNA 1490 | 74.4 | 3.5 | 63.6 | 2.8 | 72.1 | 10.4 | 72.8 | 5.4 |
| dsRNA 1491 | 52.7 | 4.9 | 68.1 | 5.4 | 45.4 | 2.8 | 82.7 | 6.2 |
| dsRNA 1492 | 54.8 | 0.9 | 67.3 | 4.0 | 56.1 | 8.8 | 88.7 | 7.5 |
| dsRNA 1493 | 77.9 | 4.5 | 74.8 | 7.8 | 69.5 | 11.9 | 98.9 | 10.7 |
| dsRNA 1494 | 57.3 | 4.8 | 80.9 | 15.2 | 58.4 | 5.2 | 93.6 | 1.5 |
| dsRNA 1495 | 67.8 | 5.0 | 82.8 | 5.6 | 85.1 | 7.5 | 95.3 | 2.0 |
| dsRNA 1496 | 55.6 | 2.0 | 85.0 | 2.7 | 56.8 | 4.1 | 89.1 | 6.3 |
| dsRNA 1497 | 99.5 | 7.3 | 70.8 | 1.8 | 88.9 | 4.9 | 76.6 | 8.1 |
| dsRNA 1498 | 95.8 | 6.5 | 59.2 | 5.8 | 87.0 | 4.6 | 72.4 | 10.5 |
| dsRNA 1499 | 74.6 | 7.6 | 56.4 | 3.5 | 63.9 | 3.3 | 76.1 | 7.7 |
| dsRNA 1500 | 49.9 | 3.7 | 82.4 | 12.9 | 56.9 | 4.9 | 71.4 | 11.9 |
| dsRNA 1501 | 50.4 | 3.1 | 57.6 | 5.8 | 53.3 | 8.3 | 77.4 | 7.4 |
| dsRNA 784 | 38.5 | 3.0 | 72.4 | 7.0 | 49.1 | 5.0 | 62.8 | 2.9 |
| dsRNA 1502 | 46.5 | 3.4 | 66.1 | 8.0 | 57.8 | 5.1 | 61.5 | 6.7 |
| dsRNA 1503 | 51.9 | 5.3 | 59.2 | 9.3 | 58.8 | 6.1 | 59.6 | 3.6 |
| dsRNA 1504 | 101.8 | 2.5 | 27.2 | 6.9 | 93.9 | 4.5 | 34.7 | 7.3 |
| dsRNA 1505 | 54.6 | 5.9 | 78.8 | 5.0 | 52.3 | 7.5 | 84.5 | 8.3 |
| dsRNA 1506 | 67.9 | 3.1 | 76.1 | 6.8 | 55.6 | 4.8 | 83.6 | 13.0 |
| dsRNA 1507 | 65.4 | 6.1 | 72.0 | 9.4 | 60.9 | 8.3 | 80.5 | 11.0 |
| dsRNA 1508 | 72.7 | 6.4 | 59.7 | 6.1 | 73.7 | 6.6 | 60.0 | 4.7 |
| dsRNA 1509 | 61.3 | 6.4 | 68.6 | 9.0 | 67.6 | 3.7 | 71.8 | 9.5 |
| dsRNA 1510 | 73.7 | 2.2 | 78.1 | 7.4 | 81.1 | 3.0 | 79.7 | 8.6 |
| dsRNA 1511 | 49.4 | 4.3 | 86.5 | 3.3 | 53.9 | 5.9 | 86.5 | 3.1 |
| dsRNA 1512 | 52.2 | 7.3 | 93.2 | 5.9 | 55.2 | 9.4 | 77.8 | 2.9 |
| dsRNA 1513 | 56.8 | 7.7 | 81.2 | 3.8 | 59.3 | 8.5 | 70.6 | 7.7 |
| dsRNA 1514 | 53.1 | 5.1 | 68.8 | 3.3 | 57.2 | 7.7 | 70.0 | 7.1 |
| dsRNA 1515 | 51.7 | 2.8 | 71.3 | 5.9 | 56.5 | 4.8 | 72.8 | 10.4 |
| dsRNA 1516 | 45.6 | 2.8 | 62.3 | 0.9 | 49.1 | 4.9 | 78.4 | 5.9 |
| dsRNA 1517 | 54.2 | 1.5 | 55.6 | 1.3 | 53.0 | 1.6 | 75.8 | 8.7 |
| dsRNA 1518 | 57.9 | 4.6 | 62.9 | 11.5 | 63.0 | 4.6 | 59.4 | 6.8 |
| dsRNA 1519 | 69.2 | 6.2 | 58.6 | 2.2 | 71.2 | 13.3 | 82.0 | 4.3 |
| dsRNA 1520 | 87.8 | 5.9 | 54.2 | 5.7 | 82.4 | 14.9 | 79.5 | 7.3 |
| dsRNA 1521 | 57.2 | 2.3 | 62.4 | 3.7 | 58.6 | 12.0 | 85.3 | 7.7 |
| dsRNA 1522 | 68.3 | 2.9 | 61.0 | 5.5 | 66.0 | 7.2 | 78.5 | 5.1 |
| dsRNA 1523 | 62.5 | 3.1 | 68.5 | 2.4 | 66.7 | 8.0 | 83.0 | 4.6 |
| dsRNA 1524 | 69.4 | 0.8 | 86.4 | 5.0 | 71.4 | 1.6 | 78.0 | 7.6 |
| dsRNA 1525 | 85.0 | 2.5 | 73.7 | 6.7 | 77.2 | 1.4 | 71.6 | 8.9 |
| dsRNA 1526 | 70.4 | 4.1 | 63.0 | 3.9 | 62.6 | 3.4 | 68.9 | 8.8 |
| dsRNA 1527 | 69.1 | 5.8 | 57.7 | 4.0 | 61.3 | 4.4 | 70.5 | 7.5 |
| dsRNA 1528 | 83.2 | 7.4 | 43.9 | 2.5 | 71.5 | 6.4 | 61.6 | 6.7 |
| dsRNA 1529 | 76.5 | 6.8 | 48.7 | 3.6 | 65.1 | 8.8 | 70.0 | 3.4 |
| dsRNA 1530 | 66.4 | 5.8 | 49.9 | 2.9 | 54.6 | 7.6 | 80.8 | 6.1 |
| dsRNA 1531 | 61.0 | 5.4 | 60.5 | 2.1 | 49.9 | 8.7 | 85.0 | 2.3 |
| dsRNA 1532 | 70.3 | 1.7 | 64.6 | 3.2 | 65.8 | 3.8 | 86.4 | 7.3 |
| dsRNA 1533 | 64.6 | 2.9 | 85.5 | 6.8 | 74.4 | 7.0 | 87.0 | 5.2 |
| dsRNA 1534 | 74.3 | 3.2 | 77.9 | 3.6 | 70.0 | 5.3 | 77.8 | 5.0 |
| dsRNA 1535 | 64.6 | 1.8 | 73.2 | 5.5 | 62.3 | 5.0 | 80.8 | 3.7 |
| dsRNA 1536 | 80.1 | 6.1 | 68.4 | 12.8 | 76.4 | 7.9 | 74.6 | 7.1 |
| dsRNA 1537 | 64.2 | 4.9 | 66.9 | 9.3 | 60.5 | 6.8 | 75.9 | 3.0 |
| dsRNA 1538 | 64.4 | 2.4 | 54.7 | 2.7 | 66.9 | 7.1 | 73.6 | 3.0 |
| dsRNA 1539 | 81.7 | 6.8 | 59.5 | 3.4 | 74.5 | 9.2 | 84.8 | 2.8 |
| dsRNA 1540 | 43.8 | 2.6 | 60.0 | 5.0 | 45.6 | 7.7 | 104.0 | 6.7 |
| dsRNA 1541 | 58.3 | 0.7 | 67.5 | 4.7 | 54.1 | 9.0 | 103.9 | 3.9 |
| dsRNA 1542 | 57.0 | 4.6 | 66.5 | 6.2 | 51.1 | 3.5 | 95.5 | 2.6 |
| dsRNA 1543 | 65.8 | 6.8 | 82.2 | 5.9 | 66.7 | 4.3 | 102.4 | 4.7 |
| dsRNA 1544 | 55.8 | 3.8 | 89.4 | 3.4 | 59.6 | 1.1 | 88.6 | 4.5 |
| dsRNA 1545 | 76.6 | 2.7 | 73.8 | 3.1 | 64.0 | 3.0 | 84.2 | 6.8 |
| dsRNA 1546 | 70.1 | 3.1 | 75.3 | 8.7 | 63.7 | 5.7 | 78.2 | 4.1 |
| dsRNA 1547 | 60.7 | 4.9 | 63.5 | 6.0 | 56.1 | 8.5 | 82.7 | 7.3 |
| dsRNA 1548 | 63.4 | 2.6 | 62.0 | 8.0 | 58.7 | 7.2 | 79.9 | 7.0 |
| dsRNA 1549 | 56.7 | 2.3 | 60.2 | 4.2 | 56.0 | 6.9 | 85.7 | 7.3 |
| dsRNA 1550 | 62.4 | 3.2 | 55.5 | 3.5 | 57.8 | 7.5 | 86.8 | 3.2 |
| dsRNA 1551 | 55.5 | 2.5 | 54.3 | 3.7 | 54.5 | 9.3 | 81.5 | 5.7 |
| dsRNA 1552 | 68.4 | 6.4 | 66.0 | 4.1 | 65.8 | 7.7 | 86.1 | 5.3 |
| dsRNA 1553 | 60.1 | 4.8 | 86.4 | 3.9 | 72.0 | 4.9 | 88.2 | 6.1 |
| dsRNA 1554 | 61.2 | 2.7 | 91.1 | 2.6 | 69.5 | 5.2 | 90.5 | 9.8 |
| dsRNA 1555 | 57.7 | 4.1 | 82.1 | 6.3 | 72.5 | 6.2 | 97.5 | 7.9 |
| dsRNA 1556 | 62.1 | 1.6 | 68.7 | 5.3 | 64.8 | 11.0 | 94.4 | 12.9 |
| dsRNA 1557 | 65.1 | 2.5 | 65.5 | 2.7 | 65.2 | 6.8 | 100.9 | 8.0 |
| dsRNA 1558 | 70.1 | 4.7 | 61.9 | 2.9 | 63.2 | 7.2 | 93.9 | 2.2 |
| dsRNA 1559 | 74.8 | 2.3 | 58.8 | 4.0 | 64.2 | 5.0 | 100.1 | 2.7 |
| dsRNA 1560 | 94.8 | 6.4 | 54.5 | 2.0 | 69.2 | 7.0 | 92.0 | 0.9 |
| dsRNA 1561 | 80.2 | 2.8 | 74.1 | 6.3 | 76.0 | 9.2 | 108.7 | 4.7 |
| dsRNA 1562 | 52.2 | 5.0 | 83.7 | 5.6 | 53.8 | 6.6 | 115.5 | 11.5 |
| dsRNA 1563 | 52.6 | 5.7 | 94.0 | 4.1 | 54.9 | 6.6 | 111.8 | 11.8 |
| dsRNA 1564 | 70.2 | 4.9 | 88.5 | 4.2 | 59.1 | 4.3 | 90.9 | 10.6 |
| dsRNA 1565 | 69.9 | 6.9 | 72.4 | 3.4 | 57.0 | 3.7 | 88.9 | 8.1 |
| dsRNA 1566 | 58.8 | 4.1 | 67.8 | 2.3 | 52.9 | 4.5 | 93.8 | 9.3 |
| dsRNA 1567 | 65.0 | 0.5 | 64.1 | 0.7 | 56.2 | 5.5 | 93.0 | 6.5 |
| dsRNA 1568 | 64.7 | 2.6 | 69.1 | 9.7 | 55.4 | 6.9 | 96.9 | 6.4 |
| dsRNA 1569 | 72.3 | 5.6 | 66.9 | 8.7 | 57.9 | 6.5 | 101.5 | 8.4 |
| dsRNA 1570 | 69.4 | 6.8 | 63.7 | 2.6 | 56.2 | 8.4 | 97.4 | 6.9 |
| dsRNA 1571 | 73.6 | 7.3 | 64.8 | 0.9 | 57.6 | 8.0 | 96.7 | 6.7 |
| dsRNA 1572 | 75.2 | 6.3 | 51.8 | 1.7 | 63.7 | 6.3 | 74.5 | 5.6 |
| dsRNA 1573 | 65.2 | 4.4 | 78.2 | 1.2 | 64.0 | 7.0 | 94.1 | 4.7 |
| dsRNA 1574 | 64.1 | 3.1 | 73.3 | 7.3 | 67.9 | 5.3 | 105.1 | 6.8 |

### 5.4 Example 4. In Vitro RNA Agent Mediated Knockdown of hBCL11A in HUDEP2 Cells

Each of the indicated dsRNA Agents set forth in Table 2 above (unmodified) were evaluated for their ability to knockdown hBCL11A expression in vitro in HUDEP2 cells.

Briefly, HUDEP2 were seeded at 2.0e5 cells per well in a 96 well nucleofector plate. The cells were nucleofected in a dose response (3000-3 nM) with the indicated hBCL11A targeting dsRNA agent according to the manufacturer's instructions and incubated for 72 hours. The level of hBCL11A was assessed utilizing a standard bDNA assay according to the manufacturer's instructions. The expression of hBCL11A was normalized to the expression of human GUSB. Each treatment group was run in quadruplicate, the mean and standard deviation calculated.

Table 20 shows the percent of maximum inhibition of hBCL11A mRNA after treatment with the indicated RNA agent (normalized to GUSB).

TABLE 20
Maximum Inhibition of hBCL11A mRNA (Normalized to GUSB).

| RNA Agent ID | IC50 nM | % Max Inhibition |
|---|---|---|
| dsRNA Agent - 1 | 433 | 55.75 |
| dsRNA Agent - 2 | 808 | 41.25 |
| dsRNA Agent - 4 | 3 | 47.25 |
| dsRNA Agent - 5 | 798 | 69 |
| dsRNA Agent - 6 | 188 | 46.25 |
| dsRNA Agent - 7 | 409 | 41.25 |
| dsRNA Agent - 8 | 808 | 41.25 |
| dsRNA Agent - 9 | 725 | 62.5 |
| dsRNA Agent - 12 | 586 | 54.25 |
| dsRNA Agent - 16 | 372 | 62.5 |

### 5.5 Example 5. In Vitro RNA Agent Mediated Knockdown of hBCL11A in HEK293 Cells

Each of the indicated dsRNA Agents set forth in Table 5 (modified agents) and ASO Agents 52 and 53 (modified agents) set forth in Table 9 above were evaluated for their ability to knockdown hBCL11A expression in vitro in HEK293 cells.

Briefly, HEK293 cells were seeded at 2.0e5 cells per well in a standard 96 well plate. The cells were transfected with the indicated hBCL11A targeting dsRNA agent (10-0.2 nM) using Opti-MEM/LF2000 mix according to the manufacturer's instructions and incubated for 24 hours. The level of hBCL11A was assessed utilizing a standard bDNA assay according to the manufacturer's instructions. The expression of hBCL11A was normalized to the expression of human GAPDH. Each treatment group was run in quadruplicate, the mean and standard deviation calculated.

Table 21 shows the percent of maximum inhibition of hBCL11A mRNA after treatment with the indicated RNA agent (normalized to GAPDH).

TABLE 21
Maximum Inhibition of hBCL11A
mRNA (Normalized to GAPDH).

| RNA Agent ID | IC50 nM | % Max Inhibition |
|---|---|---|
| dsRNA Agent - 770 | 0.002 | 65.4 |
| dsRNA Agent - 771 | 0.009 | 72.4 |
| dsRNA Agent - 772 | 0.010 | 73.7 |
| dsRNA Agent - 773 | 0.011 | 64.7 |
| dsRNA Agent - 774 | 0.012 | 61.3 |
| dsRNA Agent - 775 | 0.013 | 61.8 |
| dsRNA Agent - 776 | 0.019 | 67.4 |
| dsRNA Agent - 777 | 0.028 | 68.3 |
| dsRNA Agent - 778 | 0.029 | 66.8 |
| dsRNA Agent - 779 | 0.029 | 64.5 |
| dsRNA Agent - 780 | 0.035 | 71.9 |
| dsRNA Agent - 781 | 0.083 | 57.4 |
| dsRNA Agent - 782 | 0.205 | 66.3 |
| dsRNA Agent - 783 | 0.211 | 59.9 |
| dsRNA Agent - 784 | 0.493 | 56.4 |
| dsRNA Agent - 785 | 0.629 | 51.5 |
| ASO Agent - 56 | 0.056 | 88.7 |
| ASO Agent - 58 | 0.083 | 85.1 |

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1. (canceled)

2. A double stranded RNA agent (dsRNA) agent for inhibiting expression of BCL11 transcription factor A (BCL11A),
wherein the dsRNA agent comprises a sense strand and an antisense strand forming a double stranded region,
wherein the nucleotide sequence of the antisense strand differs by no more than 5 nucleotides from the nucleotide sequence of any one of the antisense strands set forth in any one of Tables 2-5 or set forth in any one of SEQ ID NOS: 4-1576.

3. The dsRNA agent of claim 2, wherein the nucleotide sequence of the sense strand differs by no more than 5 nucleotides from the nucleotide sequence of the corresponding sense strand set forth in Tables 2-5 or the corresponding sense strand set forth in one of SEQ ID NOS: 1577-3166.

4.-44. (canceled)

45. An antisense oligonucleotide agent for inhibiting expression of BCL11), wherein the antisense oligonucleotide comprises at least 15 contiguous nucleotides of and differing by no more than 3 nucleotides from the nucleotide sequence of any one of the antisense oligonucleotides set forth in Table 7 or 9 or set forth in any one of SEQ ID NOS: 3167-3280.

46.-63. (canceled)

64. A conjugate comprising (i) the dsRNA agent of claim 2 and (ii) a heterologous moiety.

65.-112. (canceled)

113. A vector encoding the antisense strand of claim 2.

114. A carrier comprising the dsRNA agent of claim 2.

115.-116. (canceled)

117. A cell comprising the dsRNA agent of claim 2.

118. A pharmaceutical composition comprising the dsRNA agent of claim 2, and a pharmaceutically acceptable excipient.

119. A kit comprising the dsRNA agent of claim 2.

120. A method of delivering a dsRNA agent to a cell, the method comprising introducing into a cell the dsRNA agent of claim 2, to thereby deliver the dsRNA agent into the cell.

121.-122. (canceled)

123. A method of delivering a dsRNA agent to a subject, the method comprising administering to the subject the dsRNA agent of claim 2, to thereby deliver the dsRNA agent to the subject.

124. A method of reducing or inhibiting expression of BCL11A in a cell, the method comprising delivering into the cell the dsRNA agent of claim 2, to thereby reduce or inhibit expression of BCL11A in the cell.

125. A method of reducing or inhibiting expression of BCL11A in a cell in a subject, the method comprising administering to the subject the dsRNA agent of claim 2, to thereby reduce or inhibit expression of BCL11A in the cell in the subject.

126. A method of treating, ameliorating, or preventing a BCL11A associated disease in a subject, the method comprising administering to the subject the dsRNA agent of claim 2, to thereby treat, ameliorate, or prevent the BCL11A associated disease in the subject.

127.-131. (canceled)

132. A method of treating, ameliorating, or preventing an inherited blood disorder in a subject, the method comprising administering to the subject the dsRNA agent of claim 2, to thereby treat, ameliorate, or prevent the inherited blood disorder in the subject.

133.-134. (canceled)

135. A method of treating, ameliorating, or preventing a hemoglobinopathy in a subject, the method comprising administering to the subject the dsRNA agent of claim 2, to thereby treat, ameliorate, or prevent the hemoglobinopathy in the subject.

136. (canceled)

137. A method of treating, ameliorating, or preventing an inherited bone marrow failure syndrome in a subject, the method comprising administering to the subject the dsRNA agent of claim 2, to thereby treat, ameliorate, or prevent the inherited bone marrow failure syndrome in the subject.

138. (canceled)

139. A method of inducing expression of fetal hemoglobin in a subject, the method comprising administering to the subject the dsRNA agent of claim 2, to thereby induce expression of fetal hemoglobin the subject.

140. A method of increasing the level of fetal hemoglobin in a subject, the method comprising administering to the subject the dsRNA agent of claim 2, to thereby increase the level of fetal hemoglobin the subject.

141. A method of increasing the ratio of fetal hemoglobin to adult hemoglobin in a subject, the method comprising administering to the subject the dsRNA agent of claim 2, to thereby increase the ratio of fetal hemoglobin to adult hemoglobin in the subject.

142. (canceled)
